(19) United States
(12) Patent Application Publication
(10) Pub. No.: US 2025/0320559 A1
(43) Pub. Date: Oct. 16, 2025

(54) METHODS RELATING TO IMPAIRED RESPIRATORY HEALTH THERAPEUTICS

(71) Applicants: Northwestern University, Evanston, IL (US); Vanderbilt University, Nashville, TN (US); The Brigham and Women's Hospital, Inc., Boston, MA (US)

(72) Inventors: Ravi KALHAN, Evanston, IL (US); Bina CHOI, Boston, MA (US); George R. WASHKO, Boston, MA (US); Raul SAN JOSE ESTEPAR, Boston, MA (US); Gabrielle Yi-Hui LIU, Evanston, IL (US); Andrew PERRY, Nashville, TN (US); Ravi SHAH, Nashville, TN (US)

(21) Appl. No.: 19/002,911

(22) Filed: Dec. 27, 2024

Related U.S. Application Data
(60) Provisional application No. 63/615,089, filed on Dec. 27, 2023.

(51) Int. Cl. C12Q 1/6883; G01N 33/68

(52) U.S. Cl. CPC C12Q 1/6883; G01N 33/6869; G01N 33/6884; G01N 33/6887; G01N 33/6893; G01N 2333/4704; G01N 2333/4712; G01N 2333/525; G01N 2333/65; G01N 2333/70564; G01N 2333/7155; G01N 2333/7158; G01N 2333/90212; G01N 2333/96455; G01N 2800/12

(57) ABSTRACT

The present disclosure provides include kits, compositions, and methods related to impaired respiratory health (e.g., diseases and conditions relating to lung injury). In particular, the present disclosure provides include kits, compositions, and methods for quantifying protein biomarkers associated with impaired respiratory health and assessing the risk of, monitoring, treating and/or preventing, interstitial lung diseases (ILDs).

### CROSS-REFERENCE TO RELATED APPLICATIONS

This application claims the benefit of U.S. Provisional Patent Application No. 63/615,089, filed Dec. 27, 2023; which is incorporated by reference herein in its entirety.

### FIELD

The present disclosure provides include kits, compositions, and methods related to impaired respiratory health (e.g., diseases and conditions relating to lung injury). In particular, the present disclosure provides include kits, compositions, and methods for quantifying biomarkers (e.g., proteins, nucleic acids, etc.) associated with impaired respiratory health and assessing the risk of, monitoring, treating and/or preventing, interstitial lung diseases (ILDs).

### BACKGROUND

Understanding the complex biochemical processes that regulate lung and immune system development which, subsequently, affects lung health is important to the primary prevention of lung diseases. Prior studies have shown that the presence and progression over time of quantitative interstitial abnormalities (QIA), which are subtle parenchymal changes found on chest computed tomography (CT) scans by an automated machine learning algorithm, are clinically meaningful and are associated with impaired respiratory health. For example, QIA shares some radiologic findings, risk factors, and outcomes with advanced pulmonary diseases like pulmonary fibrosis and chronic obstructive pulmonary disease (COPD). QIA, therefore, is an automated measurement to identify people at risk of progression to advanced parenchymal remodeling like fibrosis and emphysema, for which current treatments only slow down disease progression and symptoms, but do not reverse disease.

### SUMMARY

The present disclosure provides include kits, compositions, and methods related to impaired respiratory health (e.g., diseases and conditions relating to lung injury). In particular, the present disclosure provides include kits, compositions, and methods for quantifying biomarkers (e.g., proteins, nucleic acids, etc.) associated with impaired respiratory health and assessing the risk of, monitoring, treating and/or preventing, interstitial lung diseases (ILDs).

Embodiments of the present disclosure include a method comprising quantifying, within a biological sample from a subject that is a current or former tobacco smoker, the levels of two or more biomarkers from a first panel of protein biomarkers comprising: Complement factor H; Fibrinogen; Complement factor B; Antileukoproteinase; Neurofascin; Neuropilin-2; Fibrinogen gamma chain; Insulin-like growth factor-binding protein 4; C—X—C motif chemokine 16; Atrial natriuretic factor; Serine/arginine-rich splicing factor 7; Tumor necrosis factor ligand superfamily member 15; Serine/arginine-rich splicing factor 6; RNA-binding protein EWS; Immunoglobulin G; Serpin B8; Leukocyte immunoglobulin-like receptor subfamily A member 5; Cystatin-C; Nidogen-1; Gamma-glutamyl hydrolase; Complement C1q tumor necrosis factor-related protein 1; Insulin-like growth factor-binding protein 7; Plastin-2; Desmocollin-2; Ganglioside GM2 activator; Fibulin-5; Ephrin-A4; Heat shock 70 kDa protein 1B; Spondin-1; Acidic leucine-rich nuclear phosphoprotein 32 family member B; Peptidyl-prolyl cis-trans isomerase C; B melanoma antigen 2; Collagen alpha-1(XXVIII) chain; Oncoprotein-induced transcript 3 protein; Collagen alpha-3(VI) chain:Bovine pancreatic trypsin inhibitor/Kunitz inhibitor domain, isoform 1; Transmembrane emp24 domain-containing protein 10; Delta-like protein 1; Endoplasmic reticulum lectin 1; C—C motif chemokine 21; Tumor necrosis factor receptor superfamily member 17; Thioredoxin domain-containing protein 5; Beta-2-microglobulin; Heat shock cognate 71 kDa protein; WAP four-disulfide core domain protein 2; Heat shock 70 kDa protein 1A; Tolloid-like protein 1; Triggering receptor expressed on myeloid cells 1; Calsyntenin-3; Lipopolysaccharide-binding protein; Lamina-associated polypeptide 2, isoforms beta/gamma; Erythropoietin; Ephrin type-B receptor 2; C—X—C motif chemokine 13; Tryptophan—tRNA ligase, cytoplasmic; Folate receptor beta; Holo-Transcobalamin-2; NTF2-related export protein 1; Protein ABHD14A; C—C motif chemokine 18; Semaphorin-6B; Retinoic acid receptor responder protein 2; Plexin-D1; Troponin T, cardiac muscle; Macrophage-capping protein; Syntaxin-12; Interleukin-9; CUB domain-containing protein 1; Vesicular integral-membrane protein VIP36; Fibroblast growth factor 23; Heat shock 70 kDa protein 1A; BMP and activin membrane-bound inhibitor homolog:Extracellular domain; Marginal zone B- and B1-cell-specific protein; Leukocyte immunoglobulin-like receptor subfamily A member 5; Interferon-induced GTP-binding protein Mx1; Sushi, von Willebrand factor type A, EGF and pentraxin domain-containing protein 1:EGF-like domains 4-6; Heat shock 70 kDa protein 1A; Intercellular adhesion molecule 1; Ephrin type-A receptor 2; Transferrin receptor protein 1:Extracellular domain; Trefoil factor 3; Kallikrein-10; Alpha-1-antichymotrypsin complex; Neuroblastoma suppressor of tumorigenicity 1; Vascular endothelial growth factor D; Small ubiquitin-related modifier 2; T-cell immunoglobulin and mucin domain-containing protein 4; Ribonuclease K6; IGF-like family receptor 1; Sushi, von Willebrand factor type A, EGF and pentraxin domain-containing protein 1:Sushi 15-18; Elafin; ADAMTS-like protein 2; Transformer-2 protein homolog beta; Heat shock 70 kDa protein 1A; Protein CYR61; Lysozyme C; Guanylate-binding protein 1; Macrophage metalloelastase; Angiopoietin-2; Axin-2; WAP four-disulfide core domain protein 1; Complement C3d fragment; DnaJ homolog subfamily B member 12; Prostaglandin-H2 D-isomerase; Interleukin-1 receptor antagonist protein; Programmed cell death protein 5; Regenerating islet-derived protein 3-alpha; Lactoperoxidase; SCP2 sterol-binding domain-containing protein 1; Trefoil factor 3; C—C motif chemokine 19; Granulocyte colony-stimulating factor; Phospholipase A2, membrane associated; Trefoil factor 2; Gamma-interferon-inducible protein 16:Isoform 2, Hematopoietic expression, interferon-inducible nature, and nuclear localization 1; Acyl-CoA-binding protein; Thioredoxin reductase 1, cytoplasmic; Interleukin-3 receptor subunit alpha; Thrombospondin-2; Tumor necrosis factor receptor superfamily member 1B; Golgi membrane protein 1; Growth/differentiation factor 15; Kallikrein-13; Induced myeloid leukemia cell differentiation protein Mcl-1; E-selectin; Macrophage scavenger receptor types I and II:Extracellular domain; Ribonuclease pancreatic; Gastrokine-2; Receptor-transporting protein 4; Ubiquitin-like protein ISG15; C—C motif chemokine 15; Copper transport protein ATOX1; C—X—C motif chemokine 10; 60S ribosomal protein L30; Troponin I, fast skeletal muscle; Cadherin-11:Extracellular domain; Syntaxin-8; Fibrinogen-like protein 1; Serum amyloid A-2 protein; Chitotriosidase-1; Platelet-derived growth factor receptor-like protein; BPI fold-containing family B member 1; N-terminal pro-BNP; Beta-defensin 4A; C-reactive protein; Serum amyloid A-1 protein; Zymogen granule membrane protein 16; Signal peptide, CUB and EGF-like domain-containing protein 1; Glypican-3; Chondrocalcin; Protein MENT; Histone-lysine N-methyltransferase EHMT2; Sodium/potassium-transporting ATPase subunit beta-1; Neurocan core protein; Cartilage intermediate layer protein 2; Group XIIB secretory phospholipase A2-like protein; Ficolin-3; Fibroblast growth factor receptor 3:Extracellular domain; Muellerian-inhibiting factor; Solute carrier family 22 member 16; Interleukin-6 receptor subunit alpha; Proprotein convertase subtilisin/kexin type 7; Anthrax toxin receptor 1; Inositol monophosphatase 3; Thrombin; Neural cell adhesion molecule 1, 120 kDa isoform; Apolipoprotein M; Receptor-type tyrosine-protein phosphatase delta; Cell adhesion molecule-related/down-regulated by oncogenes; Ciliary neurotrophic factor receptor subunit alpha; E3 ubiquitin-protein ligase RNF146; N-acetylmuramoyl-L-alanine amidase; Matrix-remodeling-associated protein 8:Extracellular domain; Growth/differentiation factor 2; Platelet endothelial aggregation receptor 1:Extracellular domain; Myosin regulatory light chain 2, atrial isoform; Hedgehog-interacting protein; Carbonic anhydrase 4; Prostaglandin F2 receptor negative regulator; E3 ubiquitin-protein ligase DTX1; Interleukin-1 receptor type 2; Receptor-type tyrosine-protein phosphatase eta; NT-3 growth factor receptor; Leucine-rich repeats and immunoglobulin-like domains protein 3; Beta-1,4-galactosyltransferase 6; Semaphorin-3G; Plasma kallikrein; Collagen alpha-1(XIII) chain; Alpha-2-HS-glycoprotein; Bifunctional heparan sulfate N-deacetylase/N-sulfotransferase 1; Contactin-1; Cholinesterase; Heparan-sulfate 6-O-sulfotransferase 3; Coxsackievirus and adenovirus receptor; B melanoma antigen 3; BDNF/NT-3 growth factors receptor; Contactin-4; N-acetylglucosamine-1-phosphodiester alpha-N-acetylglucosaminidase; Neuropeptide S; Alpha-2-antiplasmin; Mannosyl-oligosaccharide 1,2-alpha-mannosidase IB; and Epidermal growth factor receptor.

Embodiments of the present disclosure also include a method comprising quantifying, within a biological sample from a subject that is not a current or former tobacco smoker, the levels of two or more biomarkers from a second panel of protein biomarkers comprising: HLA class I histocompatibility antigen, alpha chain G; Receptor-interacting serine/threonine-protein kinase 2; Lumican; Matrix Gla protein; Heat shock 70 kDa protein 1A; Switch-associated protein 70; Heat shock 70 kDa protein 1A; Tumor necrosis factor ligand superfamily member 15; Insulin-like growth factor-binding protein 4; Ran-binding protein 3; Laminin subunit gamma-2; C—X—C motif chemokine 13; Thrombospondin-4; Tripeptidyl-peptidase 1; Macrophage-capping protein; Thrombospondin-3; Myosin light chain 6B; Torsin-1A-interacting protein 1:Perinuclear domain; Calsyntenin-3; Lamina-associated polypeptide 2, isoforms beta/gamma; Aldose reductase; Kallikrein-10; Programmed cell death protein 5; Interleukin-12 subunit beta; 60S ribosomal protein L12; C—C motif chemokine 14; CUB domain-containing protein 1; Malignant T-cell-amplified sequence 1; Troponin T, cardiac muscle; Tudor-interacting repair regulator protein; C—X—C motif chemokine 10; Thioredoxin reductase 1, cytoplasmic; E-selectin; Tapasin; Troponin I, fast skeletal muscle; Cadherin-11:Extracellular domain; Zymogen granule membrane protein 16; CD209 antigen; Interleukin-6 receptor subunit alpha; Platelet-activating factor acetylhydrolase; Matrix-remodeling-associated protein 8:Extracellular domain; Sodium-coupled monocarboxylate transporter 1; tRNA (guanine-N(7)-)-methyltransferase; Insulin-like growth factor-binding protein 3; Insulin-like growth factor-binding protein complex acid labile subunit; Histone acetyltransferase type B catalytic subunit; Vitamin K-dependent protein C; Cytoskeleton-associated protein 2; and Afamin.

In some embodiments, the two or more biomarkers are selected from a third panel of biomarkers comprising: Insulin-like growth factor-binding protein 4; Tumor necrosis factor ligand superfamily member 15; Heat shock 70 kDa protein 1A; Calsyntenin-3; Lamina-associated polypeptide 2, isoforms beta/gamma; C—X—C motif chemokine 13; Troponin T, cardiac muscle; Macrophage-capping protein; CUB domain-containing protein 1; Kallikrein-10; Programmed cell death protein 5; Thioredoxin reductase 1, cytoplasmic; E-selectin; C—X—C motif chemokine 10; Troponin I, fast skeletal muscle; Cadherin-11:Extracellular domain; Zymogen granule membrane protein 16; Interleukin-6 receptor subunit alpha; and Matrix-remodeling-associated protein 8:Extracellular domain.

In some embodiments, quantifying comprises a biophysical technique.

In some embodiments, the biophysical technique comprises using a point-of-care device.

In some embodiments, the point-of-care device uses protein biomarkers binding agents and a detection/quantification methodology.

In some embodiments, the protein biomarkers binding agents are antibodies, antibody fragments, or aptamers.

In some embodiments, the detection/quantification methodology is fluorescence, luminescence, or electrochemical detection.

In some embodiments, the biological sample is plasma or blood.

In some embodiments, the biological sample is obtained from a subject that suffers from impaired respiratory health.

In some embodiments, the protein biomarkers are associated with a quantitative measure of impaired respiratory health.

In some embodiments, the .quantitative measure of impaired respiratory health comprises quantitative CT-based measurement of early lung injury.

In some embodiments, the protein biomarkers; are associated with advanced parenchymal diseases.

In some embodiments, the advanced parenchymal diseases comprise lung injury, inflammation, pulmonary fibrosis, COPD and emphysema, and lung cancer

In some embodiments, the subject has received treatment with a chest computed tomography (CT) scan.

Embodiments of the present disclosure also include a method of assessing the lung health of a subject comprising: (a) quantifying levels of a panel of protein biomarkers in a biological sample from the subject according to the method of one of claims 1-16; and (b) comparing the level of each protein biomarker in the fourth panel to a control or threshold value for each, wherein a significant difference between the level of one or more of the protein biomarkers from the control or threshold value is indicative of a impaired respiratory health. In some embodiments, the method further comprises measuring quantitative interstitial abnormalities (QIA) with a CT scan of the subject.

In some embodiments, the panel comprises 50 or fewer protein biomarkers.

In some embodiments, the panel comprises 20 or fewer protein biomarkers.

In some embodiments, the control or threshold value is based on a population average for healthy subjects.

In some embodiments, comparing the level of each protein biomarker in the panel to a control or threshold value for each comprises calculating the difference between the level of each protein biomarker in the panel to a control or threshold value for each.

In some embodiments, comparing the level of each protein biomarkers in the panel to a control or threshold value for each further comprises dividing the difference by the standard deviation for the population of healthy subjects to generate a protein biomarkers score for each protein biomarker.

In some embodiments, the method further comprises generating a composite score for the panel of protein biomarkers by comparing the level of each protein biomarker in the panel to a control or threshold value for each to generate a protein biomarkers score for each protein biomarker and combining the protein biomarkers scores to generate the composite score; and comparing the composite score to a composite threshold value, wherein a significant difference between the composite score to a composite threshold value is indicative of a impaired respiratory health.

In some embodiments, the control or threshold value for each protein biomarkers is based on a population average for healthy subjects.

In some embodiments, the protein biomarkers score for each protein biomarker in the panel is calculated by calculating the difference between the level of each protein biomarker in the panel to a control or threshold value for each and dividing the difference by the standard deviation for the population of healthy subjects.

### BRIEF DESCRIPTION OF THE DRAWINGS

FIGS. 1A-1B: In FIG. 1A is an example computed tomography (CT) cut, with, in FIG. 1B, the quantitative interstitial abnormalities (QIA) shaded.

FIG. 2: In FIG. 2 is a volcano plot of proteins significantly associated with QIA in CARDIA. Proteins are in blue if significant in the CARDIA cohort and in red if significant in both CARDIA and COPDGene.

FIG. 3: In FIG. 3 are proteins that are significantly associated with QIA in CARDIA and COPDGene, with beta coefficients of associations in CARDIA on the x-axis and in COPDGene on the y-axis.

FIG. 4: In FIG. 4 is a pathway enrichment analysis of proteins associated with QIA shared in COPDGene and CARDIA participants.

FIGS. 5A-5B: In FIG. 5 are results of the tissue-specific deconvolution of the significant proteins using the Genotype-Tissue Expression (GTEx) database. The gene expression activities of the proteins positively significant for QIA that were scored by the R package singscore for each tissue, which were used to rank the 37 tissues. FIG. 5A shows CARDIA and FIG. 5B shows COPDGene.

FIG. 6: In FIG. 6 are principal components 1 and 2 of proteomics in CARDIA, colored by study centers.

### DETAILED DESCRIPTION

Embodiments of present disclosure provide include kits, compositions, and methods related to impaired respiratory health (e.g., diseases and conditions relating to lung injury). In particular, the present disclosure provides include kits, compositions, and methods for quantifying biomarkers (e.g., proteins, nucleic acids, etc.) associated with impaired respiratory health and assessing the risk of, monitoring, treating and/or preventing, interstitial lung diseases (ILDs).

Experiments were conducted during development of embodiments herein using a large-scale proteomics panel to uncover protein biomarkers associated with QIA, a quantitative CT-based measurement of early impaired respiratory health. It was found that 144 unique proteins associated with QIA were shared across two cohorts that spanned middle-age to older-adulthood. These proteins included biomarkers not previously associated with impaired respiratory health, as well as those previously associated with inflammation, pulmonary fibrosis, COPD and emphysema, and lung cancer, as described below, thus suggesting potential shared pathways between QIA and advanced parenchymal diseases. Many of these associations were present in the younger cohort of community-based adults with varied smoking histories, including never-smokers.

Identified and described herein are the proteomic biomarkers (and nucleic acids encoding such protein biomarkers) of QIA that are present both in an older, smoking population with a higher prevalence of pulmonary disease, and in a younger, healthier, community-based cohort representing an “upstream” population earlier in the life course. All but one of the 144 unique shared proteins had the same direction and relative magnitude of associations between the two cohorts, showing that these proteins have similar associations with QIA even though the CARDIA cohort is younger and less enriched for smoking exposure and parenchymal disease. Such associations with QIA suggest that insults to the lung and disease pathogenesis start years before the manifestation of chronic lung disease, and that systemic biologic markers of injury may be detectable and important even without the clinical or radiographic presence of advanced, severe disease. Combined with early QIA imaging findings, these protein biomarkers have use not only in the early detection of the precursors disease, but also in the development of targets for timely intervention and therapeutics.

Forty-nine proteins were associated with QIA in the never-smoker subset. These associations indicate that, although smoking is an important risk factor for QIA, it is not the only source of injury and inflammation, and further support the idea that QIA is a heterogeneous condition encompassing several subtypes and pathways of progression. Protein biomarkers (and nucleic acids encoding such protein biomarkers) are especially valuable in the detection and stratification of patients with QIA that do not have the classic risk factors of heavy smoking use or advanced age but are nonetheless at high risk of advanced lung disease. In addition, by validating these associations within the two cohorts, 184 proteins were identified with more important associations with the QIA of older ever-smokers.

In experiments conducted during development of embodiments herein, 144 proteomic biomarkers and 49 enriched pathways associated with QIA were identified in an older smoking population and a younger community-based population. The identified proteins and pathways provide biological insight into QIA, and are useful as biomarkers to improve detection of early precursors of advanced parenchymal diseases and as therapeutic targets for disease interception.

### 1. Definitions

Unless otherwise defined, all technical and scientific terms used herein have the same meaning as commonly understood by one of ordinary skill in the art. The meaning and scope of the terms should be clear; in the event, however of any latent ambiguity, definitions provided herein take precedent over any dictionary or extrinsic definition. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting. Unless otherwise required by context, singular terms shall include pluralities and plural terms shall include the singular.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

As used herein, the term “subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (e.g., a monkey, such as a cynomolgus or rhesus monkey, chimpanzee, macaque, etc.) and a human). In some embodiments, the subject may be a human or a non-human. In one embodiment, the subject is a human. The subject or patient may be undergoing various forms of treatment.

As used herein, the term “treat,” “treating” or “treatment” are each used interchangeably herein to describe reversing, alleviating, or inhibiting the progress of a disease and/or injury, or one or more symptoms of such disease, to which such term applies. Depending on the condition of the subject, the term also refers to preventing a disease, and includes preventing the onset of a disease, or preventing the symptoms associated with a disease (e.g., viral infection). A treatment may be either performed in an acute or chronic way. The term also refers to reducing the severity of a disease or symptoms associated with such disease prior to affliction with the disease. Such prevention or reduction of the severity of a disease prior to affliction refers to administration of a treatment to a subject that is not at the time of administration afflicted with the disease. “Preventing” also refers to preventing the recurrence of a disease or of one or more symptoms associated with such disease.

As used herein, “an individual is suspected of being susceptible at risk for AD” is meant to refer to an individual who is at an above-average risk of developing AD (AD).

Examples of individuals at a particular risk of developing AD are those whose family medical history indicates above average incidence of AD, individuals of advanced age, individuals exhibiting signs or symptoms of MCI or SCI. Other factors which may contribute to an above-average risk of developing AD may be based upon an individual's specific genetic, medical, psychological, psychosocial, and/or behavioral background and characteristics.

As used herein, the term “specificity” is defined as a statistical measure of performance of an assay (e.g., method, test), calculated by dividing the number of true negatives by the sum of true negatives and false positives.

As used herein, the term “informative” or “informativeness” refers to a quality of a marker or panel of markers, and specifically to the likelihood of finding a marker (or panel of markers) in a positive sample.

As used herein, the term “sample” is used in its broadest sense. For example, in some embodiments, it is meant to include a specimen (e.g., blood sample, cerebrospinal fluid (CSF) sample). In preferred embodiments, it is meant to include a biological sample. The present invention is not limited by the type of biological sample used or analyzed. The present invention is useful with a variety of biological samples including, but are not limited to, tissue (e.g., organ (e.g., heart, liver, brain, lung, stomach, intestine, spleen, kidney, pancreas, and reproductive (e.g., ovaries) organs; lung biopsy), glandular, skin, and muscle tissue), cell (e.g., blood cell (e.g., lymphocyte or erythrocyte), muscle cell, tumor cell, bronchial cell, bronchioalveolar cells, and skin cell), gas, bodily fluid (e.g., tracheal aspirate fluid, bronchoalveolar fluid, bronchoalveolar lavage sample, blood or portion thereof, serum, plasma, urine, semen, saliva, etc), or solid (e.g., stool) samples obtained from a human (e.g., adult, infant, or embryo) or animal (e.g., cattle, poultry, mouse, rat, dog, pig, cat, horse, and the like). Biological samples may be obtained from all of the various families of domestic animals, as well as feral or wild animals, including, but not limited to, such animals as ungulates, bear, fish, lagamorphs, rodents, etc. Biological samples also include biopsies and tissue sections (e.g., biopsy or section of tumor, growth, rash, infection, or paraffin-embedded sections), medical or hospital samples (e.g., including, but not limited to, bronchoalveolar lavage fluid (BAL) samples, tracheal aspirate fluid, blood samples, saliva, buccal swab, cerebrospinal fluid, pleural fluid, milk, colostrum, lymph, sputum, vomitus, bile, semen, oocytes, cervical cells, amniotic fluid, urine, stool, hair and sweat), and laboratory samples (e.g., subcellular fractions).

As used herein, the term “sensitivity” is defined as a statistical measure of performance of an assay (e.g., method, test), calculated by dividing the number of true positives by the sum of the true positives and the false negatives.

### 2. Sample Analysis, Biomarker Selection, and Assessment of Lung Health

Embodiments of the present disclosure include kits, compositions, and methods for identifying, selecting, and analyzing (e.g., quantifying) at least one (e.g., one or more) biomarker (e.g., a protein (e.g., an exogenous or endogenous biopolymeric structure composed of amino acids (e.g., antibodies, contractile proteins, enzymes, hormonal proteins, structural proteins, storage proteins, and transport proteins) and/or their genetic elements (e.g., genomes)))). In some embodiments, a biomarker is associated with a molecular function, a cellular component, and/or a biological process. In some embodiments, the level of at least one (e.g., one or more) prognostic biomarker in a sample from a subject is indicative/prognostic/diagnostic of a condition/outcome in the subject (e.g., a prognostic biomarker).

Experiments were conducted during development of embodiments herein to identify a panel of biomarkers that when quantified individually and/or collectively provide information about the health status of a subject (e.g., health and/or makeup of the subject's respiratory health) and/or a treatment course of action to maintain or enhance the health of the subject. In some embodiments, methods of identifying such biomarkers are within the scope herein.

In some embodiments, a method for measuring QIA and/or impaired respiratory health (e.g., diseases and conditions relating to lung injury (e.g., infection, cancer, blocked blood flow in the lung (e.g., pulmonary embolism), etc.)) includes thoracic CT scans (e.g., inspiratory volumetric CT scans) of a subject's chest and/or a histogram classifier (e.g., a local or a global histogram classifier).

In some embodiments, a biological sample (e.g., blood or plasma) from a subject is collected. In some embodiments, venous blood from a subject is collected in a collection vessel (e.g., EDTA tubes) and centrifuged and separated into plasma. In some embodiments, the venous blood from a subject is centrifuged and separated into plasma within 2 hours of collection. In some embodiments, the plasma is stored in aliquots (e.g., 0.5 ml) at −80° C.

In some embodiments, a method for identifying a biomarker includes an aptamer-based proteomics assay capable of measuring human protein analytes in serum, plasma, and other biological matrices with high sensitivity and specificity is used. In some embodiments, a chemically modified single-stranded DNA aptamer that binds to epitopes on a target protein and reagents are then hybridized to complementary sequences on a DNA microarray in the Assay to yield relative fluorescence units. In some embodiments, an aptamer-based proteomics assay is a SOMAscan assay. In some embodiments, an aliquot (e.g., 130 uL) of plasma is used for proteomics analysis. For additional information regarding aptamer-based proteomics assays, see, e.g., Candia, J., Cheung, F., Kotliarov, Y. et al. Assessment of Variability in the SOMAscan Assay. Sci Rep 7, 14248 (2017). doi.org/10.1038/s41598-017-14755-5; incorporated by reference in its entirety. In some embodiments, standard processes (e.g., plate hybridization, median signal normalization, plate scaling, and calibration) are performed independently on all samples. In some embodiments, protein levels are reported in relative fluorescent units (RFU) and natural log-transformed for analysis.

In some embodiments, proteins were identified by assessing the association between each log-transformed protein as a continuous measure and QIA (percent QIA in lung) as a continuous measure with univariate linear regression, followed by multivariable linear regression adjusted for age, sex, body mass index (BMI), smoking status (current/former/never), and study center as a random effect to adjust for technical variation.

In some embodiments, pathway enrichment analysis of identified proteins (e.g., a computational biology method to identify biological functions that are overrepresented (e.g., more than would be expected by chance) in a group of genes and rank by relevance) was performed. In some embodiments, pathway enrichment analysis of an identified protein (e.g., a significant protein) was performed to link the identified protein with impaired respiratory health and/or other diseases and conditions. In some embodiments, tissue-specific deconvolution of the significant proteins was performed to link and score the gene expression activities of a protein positively significant for impaired respiratory health and/or other diseases and conditions.

Embodiments of the present disclosure include methods of analyzing a biological sample (e.g., blood or plasma sample) from a subject to isolate and/or detect and/or determine (quantitatively) the levels of various biomarkers (e.g., protein biomarkers, nucleic acids encoding protein biomarkers, etc.).

In some embodiments, a biomarker (e.g., protein biomarkers, nucleic acids encoding protein biomarkers, etc.) screen is performed on a sample (e.g., biological sample) from a subject to identify, quantify, etc. various biomarkers present. In some embodiments, a biomarker (e.g., protein biomarkers, nucleic acids encoding protein biomarkers, etc.) screen is performed to detect and/or quantify as many biomarkers as are detectable by the methods used. In some embodiments, one or more key biomarkers (e.g., protein biomarkers identified herein, nucleic acids encoding protein biomarkers, etc.) are detected and/or quantified. In some embodiments, 2 or more biomarkers are detected and/or quantified (e.g., 2, 3, 4, 5, 6, 7, 8, 9, 10, 12, 15, 20, 25, 30, 35, 40, 45, 50, 75, 100, or more). In some embodiments, 100 or fewer biomarkers are detected and/or quantified (e.g., 100, 80, 60, 50, 40, 30, 20, 10, 5, or fewer). In some embodiments, 1-20 (e.g., 1, 2, 3, 4, 5, 6, 7, 8, 9, 10, 11, 12, 13, 14, 15, 16, 17, 18, 19, 20) of the biomarkers described herein are detected and/or quantified.

In some embodiments, a biomarker panel comprises 2 or more biomarkers (e.g., 2, 3, 4, 5, 6, 7, 8, 9, 10, 11, 12, 13, 14, 15, 16, 17, 18, 19, 20, 25, 30, 40, 50, 75, 100, 200, or more, or ranges or values therebetween). In some embodiments, a biomarker panel comprises 2 or more biomarkers selected from Table 2; Table 3; Table 4; Table 5; Table 6; Table 7; Table 8; Table 9; Table 10; Table 11; Table 12; and/or Table 13 (e.g., 2, 3, 4, 5, 6, 7, 8, 9, 10, 11, 12, 13, 14, 15, 16, 17, 18, 19, 20, or ranges or values therebetween). In some embodiments, a biomarker panel comprises 100 or fewer biomarkers (e.g., 100, 90, 80, 70, 60, 50, 40, 30, 25, 20, 19, 18, 17, 16, 15, 14, 13, 12, 11, 10, 9, 8, 7, 6, 5, 4, or ranges or values therebetween). In some embodiments, a biomarker panel comprises 20 or fewer biomarkers selected from Table 2; Table 3; Table 4; Table 5; Table 6; Table 7; Table 8; Table 9; Table 10; Table 11; Table 12; and/or Table 13 (e.g., 20, 19, 18, 17, 16, 15, 14, 13, 12, 11, 10, 9, 8, 7, 6, 5, 4, 3, 2, or ranges or values therebetween). In some embodiments, a panel of biomarkers is selected from one or more key biomarkers (e.g., protein biomarkers identified herein, nucleic acids encoding protein biomarkers, etc.). In some embodiments, 2 or more panels of biomarkers are detected and/or quantified (e.g., 2, 3, 4, 5, 6, 7, 8, 9, 10, 12, 15, 20, 25, 30, 35, 40, 45, 50, 75, 100, or more). In some embodiments, 100 or fewer panels of biomarkers are detected and/or quantified (e.g., 100, 80, 60, 50, 40, 30, 20, 10, 5, or fewer). In some embodiments, 1-20 (e.g., 1, 2, 3, 4, 5, 6, 7, 8, 9, 10, 11, 12, 13, 14, 15, 16, 17, 18, 19, 20) of the panels of biomarkers described herein are detected and/or quantified.

In some embodiments, the biomarkers herein are detected as proteins or other biomarkers (e.g., nucleic acids) in a sample from a subject. In some embodiments, any technique and/or instrumentation suitable for detecting/quantifying proteins or other biomarkers (e.g., nucleic acids) in a complex environment may find use in embodiments herein. In some embodiments, a point-of-care (POC) device is utilized for detecting/quantifying biomarkers in a complex environment. In some embodiments, a POC device allows for detection/quantification of biomarkers at a clinic, hospital, or other testing facility accessible or near to a patient, at reduced cost compared to traditional instruments, and in a short time span. In some embodiments, a POC device utilizes biosensors comprising antibodies, antibody, fragments, aptamers, etc. for binding to biomarkers and optical, fluorescent, luminescent, electrochemical, etc. detection for quantifying the protein biomarkers. Embodiments herein are not limited by the technique, POC or otherwise, used for quantification of the biomarkers. In some embodiments, detecting/quantifying biomarkers includes methods such as amplifying nucleic acids (e.g., PCR), protein separation (e.g., protein electrophoresis (e.g., sodium dodecyl sulfate polyacrylamide gel electrophoresis (SDS-PAGE)), variations of chromatic methods (e.g., laser diffraction, and chromatography (e.g., thin-layer chromatography (TLC), and column chromatography (e.g., high-performance liquid-based chromatography (HPLC), ultra-performance liquid-based chromatography (UPLC), ion exchange column chromatography, hydrophobic interaction column chromatography), size exclusion chromatography, and affinity chromatography))), and immunoblotting (e.g., dot blotting, quantitative enzyme-linked immunosorbent assays (ELISA), and Western blotting (e.g., Colorimetric blotting, Fluorometric blotting, and Chemiluminescent blotting)) and protein identification (e.g., Edman Degradation, Nuclear Magnetic Resonance (NMR) spectroscopy, and protein mass spectrometry (e.g., peptide mass fingerprinting and de novo peptide sequencing).

In some embodiments, the biomarkers herein are detected as nucleic acids encoding the protein biomarkers identified in the experiments conducted during development of embodiments herein. Exemplary methods for detecting the presence or absence of a nucleic acid biomarker (e.g., mRNA. DNA, etc.) include, but are not limited to, polymerase chain reaction (PCR)-based technologies (e.g., reverse transcription PCR (RT-PCR) and quantitative or real-time RT-PCR (RT-qPCR)). Other methods include microarray analysis. RNA sequencing (e.g., next-generation sequencing (NGS)), in situ hybridization, and Northern blot.

In some embodiments, biomarkers are detected at the nucleic acid level. For example, the presence or amount of biomarker nucleic acid (e.g., mRNA) in a sample is determined (e.g., to determine the presence or level of biomarker expression). Biomarker nucleic acid (e.g., RNA, amplified cDNA, etc.) may be detected/quantified using a variety of nucleic acid techniques known to those of ordinary skill in the art, including but not limited to nucleic acid sequencing, nucleic acid hybridization, nucleic acid amplification (e.g., by PCR. RT-PCR, qPCR, etc.), microarray. Southern and Northern blotting, sequencing, etc. Non-amplified or amplified nucleic acids can be detected by any conventional means. For example, in some embodiments, nucleic acids are detected by hybridization with a detectably labeled probe and measurement of the resulting hybrids. Nucleic acid detection reagents may be labeled (e.g., fluorescently) or unlabeled, and may by free in solution or immobilized (e.g., on a bead, well, surface, chip, etc.).

In some embodiments, a method for measuring QIA and/or impaired respiratory health (e.g., diseases and conditions relating to lung injury (e.g., infection, cancer, blocked blood flow in the lung (e.g., pulmonary embolism), etc.)) includes thoracic CT scans (e.g., inspiratory volumetric CT scans) of a subject's chest and/or a histogram classifier (e.g., a local or a global histogram classifier). In some embodiments, the biomarkers and/or panels thereof and/or levels thereof that are provided herein correlate with normal respiratory health (e.g., relative to the general healthy population). In some embodiments, biomarkers present within a range relative to a population average and/or reference range indicates normal (e.g., not atypically high, not atypically low) levels of respiratory health (e.g., lack of diseases and conditions relating to lung injury). In some embodiments, biomarkers that are beyond a threshold different (e.g., higher) than a population average and/or reference range indicates abnormal (e.g., atypically high, atypically low) levels of respiratory health (e.g., potential for diseases and conditions relating to lung injury).

Experiments conducted during development of embodiments herein demonstrate that the levels of various protein biomarkers (or nucleic acids encoding such biomarkers) present in biological samples (e.g., plasma or blood) from a subject correlate to respiratory health of the subject and are diagnostic of the health of the subject. In some embodiments, provided herein are panels of biomarkers (e.g., comprising one or more biomarkers selected from the group consisting of: Table 2; Table 3; Table 4; Table 5; Table 6; Table 7; Table 8; Table 9; Table 10; Table 11; Table 12; and/or Table 13), the levels of which in biological samples (e.g., blood or plasma sample) from a subject correlate with the respiratory health of the subject. In some embodiments, increased differences in the levels of one or more biomarkers of a panel (e.g., comprising proteins selected from the group consisting of: Table 2; Table 3; Table 4; Table 5; Table 6; Table 7; Table 8; Table 9; Table 10; Table 11; Table 12; and/or Table 13) from the population average, above/below a threshold, etc. it is indicative of an atypical respiratory health (e.g., diseases and conditions relating to lung injury).

In some embodiments, the presence of and/or levels of modification and/or remodeling of a number of all or a subset of the biomarkers in a biomarker panel (e.g., proteins contained in the pulmonary extracellular matrix (ECM) (e.g., collagens, elastin, fibrin, fibronectin, laminin, proteoglycans, and glycoproteins that compose the structural framework; the integrins and other cell adhesion receptors that create feedback connections between pulmonary cells and the ECM; growth factors, cytokines, and chemokines for which the ECM serves as a reservoir; and the metalloproteinases that regulate modification and degradation)) are correlated to the presence of specific lung injury of the subject. For example, if a biomarker or group of biomarkers are quantified as, beyond a threshold from the population average, outside of a reference range, etc., this may indicate increased likelihood of chronic lung diseases (e.g., pulmonary fibrosis and emphysema). In some embodiments, a number of all or a subset of the biomarkers in a biomarker panel are associated with various diseases and conditions relating to impaired respiratory health (e.g., lung progression of COPD and pulmonary fibrosis, progressive fibrosing interstitial lung disease (ILD), idiopathic pulmonary fibrosis (IPF), emphysema, cellular stress and senescence, and lung cancers).

In some embodiments, methods are provided of assessing and/or analyzing the levels of biomarkers in a biological sample (e.g., blood or plasma sample) from a subject. In some embodiments, the protein biomarkers in the panels herein and/or assessed herein include one or more biomarkers selected from the group consisting of: Table 2; Table 3; Table 4; Table 5; Table 6; Table 7; Table 8; Table 9; Table 10; Table 11; Table 12; and/or Table 13.

In some embodiments, a panel of one or more biomarkers is selected from the group consisting of: Table 2; Table 3; Table 4; Table 5; Table 6; Table 7; Table 8; Table 9; Table 10; Table 11; Table 12; and/or Table 13.

In some embodiments, the biomarkers in the panels herein and/or assessed herein include one or more biomarkers selected from the group consisting of a first panel of protein biomarkers. In some embodiments the first panel comprises: Complement factor H; Fibrinogen; Complement factor B; Antileukoproteinase; Neurofascin; Neuropilin-2; Fibrinogen gamma chain; Insulin-like growth factor-binding protein 4; C—X—C motif chemokine 16; Atrial natriuretic factor; Serine/arginine-rich splicing factor 7; Tumor necrosis factor ligand superfamily member 15; Serine/arginine-rich splicing factor 6; RNA-binding protein EWS; Immunoglobulin G; Serpin B8; Leukocyte immunoglobulin-like receptor subfamily A member 5; Cystatin-C; Nidogen-1; Gamma-glutamyl hydrolase; Complement C1q tumor necrosis factor-related protein 1; Insulin-like growth factor-binding protein 7; Plastin-2; Desmocollin-2; Ganglioside GM2 activator; Fibulin-5; Ephrin-A4; Heat shock 70 kDa protein 1B; Spondin-1; Acidic leucine-rich nuclear phosphoprotein 32 family member B; Peptidyl-prolyl cis-trans isomerase C; B melanoma antigen 2; Collagen alpha-1(XXVIII) chain; Oncoprotein-induced transcript 3 protein; Collagen alpha-3(VI) chain:Bovine pancreatic trypsin inhibitor/Kunitz inhibitor domain, isoform 1; Transmembrane emp24 domain-containing protein 10; Delta-like protein 1; Endoplasmic reticulum lectin 1; C—C motif chemokine 21; Tumor necrosis factor receptor superfamily member 17; Thioredoxin domain-containing protein 5; Beta-2-microglobulin; Heat shock cognate 71 kDa protein; WAP four-disulfide core domain protein 2; Heat shock 70 kDa protein 1A; Tolloid-like protein 1; Triggering receptor expressed on myeloid cells 1; Calsyntenin-3; Lipopolysaccharide-binding protein; Lamina-associated polypeptide 2, isoforms beta/gamma; Erythropoietin; Ephrin type-B receptor 2; C—X—C motif chemokine 13; Tryptophan—tRNA ligase, cytoplasmic; Folate receptor beta; Holo-Transcobalamin-2; NTF2-related export protein 1; Protein ABHD14A; C—C motif chemokine 18; Semaphorin-6B; Retinoic acid receptor responder protein 2; Plexin-D1; Troponin T, cardiac muscle; Macrophage-capping protein; Syntaxin-12; Interleukin-9; CUB domain-containing protein 1; Vesicular integral-membrane protein VIP36; Fibroblast growth factor 23; Heat shock 70 kDa protein 1A; BMP and activin membrane-bound inhibitor homolog:Extracellular domain; Marginal zone B- and Bi-cell-specific protein; Leukocyte immunoglobulin-like receptor subfamily A member 5; Interferon-induced GTP-binding protein Mx1; Sushi, von Willebrand factor type A, EGF and pentraxin domain-containing protein 1:EGF-like domains 4-6; Heat shock 70 kDa protein 1A; Intercellular adhesion molecule 1; Ephrin type-A receptor 2; Transferrin receptor protein 1:Extracellular domain; Trefoil factor 3; Kallikrein-10; Alpha-1-antichymotrypsin complex; Neuroblastoma suppressor of tumorigenicity 1; Vascular endothelial growth factor D; Small ubiquitin-related modifier 2; T-cell immunoglobulin and mucin domain-containing protein 4; Ribonuclease K6; IGF-like family receptor 1; Sushi, von Willebrand factor type A, EGF and pentraxin domain-containing protein 1:Sushi 15-18; Elafin; ADAMTS-like protein 2; Transformer-2 protein homolog beta; Heat shock 70 kDa protein 1A; Protein CYR61; Lysozyme C; Guanylate-binding protein 1; Macrophage metalloelastase; Angiopoietin-2; Axin-2; WAP four-disulfide core domain protein 1; Complement C3d fragment; DnaJ homolog subfamily B member 12; Prostaglandin-H2 D-isomerase; Interleukin-1 receptor antagonist protein; Programmed cell death protein 5; Regenerating islet-derived protein 3-alpha; Lactoperoxidase; SCP2 sterol-binding domain-containing protein 1; Trefoil factor 3; C—C motif chemokine 19; Granulocyte colony-stimulating factor; Phospholipase A2, membrane associated; Trefoil factor 2; Gamma-interferon-inducible protein 16:Isoform 2, Hematopoietic expression, interferon-inducible nature, and nuclear localization 1; Acyl-CoA-binding protein; Thioredoxin reductase 1, cytoplasmic; Interleukin-3 receptor subunit alpha; Thrombospondin-2; Tumor necrosis factor receptor superfamily member 1B; Golgi membrane protein 1; Growth/differentiation factor 15; Kallikrein-13; Induced myeloid leukemia cell differentiation protein Mcl-1; E-selectin; Macrophage scavenger receptor types I and II:Extracellular domain; Ribonuclease pancreatic; Gastrokine-2; Receptor-transporting protein 4; Ubiquitin-like protein ISG15; C—C motif chemokine 15; Copper transport protein ATOX1; C—X—C motif chemokine 10; 60S ribosomal protein L30; Troponin I, fast skeletal muscle; Cadherin-11:Extracellular domain; Syntaxin-8; Fibrinogen-like protein 1; Serum amyloid A-2 protein; Chitotriosidase-1; Platelet-derived growth factor receptor-like protein; BPI fold-containing family B member 1; N-terminal pro-BNP; Beta-defensin 4A; C-reactive protein; Serum amyloid A-1 protein; Zymogen granule membrane protein 16; Signal peptide, CUB and EGF-like domain-containing protein 1; Glypican-3; Chondrocalcin; Protein MENT; Histone-lysine N-methyltransferase EHMT2; Sodium/potassium-transporting ATPase subunit beta-1; Neurocan core protein; Cartilage intermediate layer protein 2; Group XIIB secretory phospholipase A2-like protein; Ficolin-3; Fibroblast growth factor receptor 3:Extracellular domain; Muellerian-inhibiting factor; Solute carrier family 22 member 16; Interleukin-6 receptor subunit alpha; Proprotein convertase subtilisin/kexin type 7; Anthrax toxin receptor 1; Inositol monophosphatase 3; Thrombin; Neural cell adhesion molecule 1, 120 kDa isoform; Apolipoprotein M; Receptor-type tyrosine-protein phosphatase delta; Cell adhesion molecule-related/down-regulated by oncogenes; Ciliary neurotrophic factor receptor subunit alpha; E3 ubiquitin-protein ligase RNF146; N-acetylmuramoyl-L-alanine amidase; Matrix-remodeling-associated protein 8:Extracellular domain; Growth/differentiation factor 2; Platelet endothelial aggregation receptor 1:Extracellular domain; Myosin regulatory light chain 2, atrial isoform; Hedgehog-interacting protein; Carbonic anhydrase 4; Prostaglandin F2 receptor negative regulator; E3 ubiquitin-protein ligase DTX1; Interleukin-1 receptor type 2; Receptor-type tyrosine-protein phosphatase eta; NT-3 growth factor receptor; Leucine-rich repeats and immunoglobulin-like domains protein 3; Beta-1,4-galactosyltransferase 6; Semaphorin-3G; Plasma kallikrein; Collagen alpha-1(XIII) chain; Alpha-2-HS-glycoprotein; Bifunctional heparan sulfate N-deacetylase/N-sulfotransferase 1; Contactin-1; Cholinesterase; Heparan-sulfate 6-O-sulfotransferase 3; Coxsackievirus and adenovirus receptor; B melanoma antigen 3; BDNF/NT-3 growth factors receptor; Contactin-4; N-acetylglucosamine-1-phosphodiester alpha-N-acetylglucosaminidase; Neuropeptide S; Alpha-2-antiplasmin; Mannosyl-oligosaccharide 1,2-alpha-mannosidase IB; and Epidermal growth factor receptor.

In some embodiments, the biomarkers in the panels herein and/or assessed herein include one or more biomarkers selected from the group consisting of a second panel of protein biomarkers. In some embodiments the second panel comprises: HLA class I histocompatibility antigen, alpha chain G; Receptor-interacting serine/threonine-protein kinase 2; Lumican; Matrix Gla protein; Heat shock 70 kDa protein 1A; Switch-associated protein 70; Heat shock 70 kDa protein 1A; Tumor necrosis factor ligand superfamily member 15; Insulin-like growth factor-binding protein 4; Ran-binding protein 3; Laminin subunit gamma-2; C—X—C motif chemokine 13; Thrombospondin-4; Tripeptidyl-peptidase 1; Macrophage-capping protein; Thrombospondin-3; Myosin light chain 6B; Torsin-1A-interacting protein 1:Perinuclear domain; Calsyntenin-3; Lamina-associated polypeptide 2, isoforms beta/gamma; Aldose reductase; Kallikrein-10; Programmed cell death protein 5; Interleukin-12 subunit beta; 60S ribosomal protein L12; C—C motif chemokine 14; CUB domain-containing protein 1; Malignant T-cell-amplified sequence 1; Troponin T, cardiac muscle; Tudor-interacting repair regulator protein; C—X—C motif chemokine 10; Thioredoxin reductase 1, cytoplasmic; E-selectin; Tapasin; Troponin I, fast skeletal muscle; Cadherin-11:Extracellular domain; Zymogen granule membrane protein 16; CD209 antigen; Interleukin-6 receptor subunit alpha; Platelet-activating factor acetylhydrolase; Matrix-remodeling-associated protein 8:Extracellular domain; Sodium-coupled monocarboxylate transporter 1; tRNA (guanine-N(7)-)-methyltransferase; Insulin-like growth factor-binding protein 3; Insulin-like growth factor-binding protein complex acid labile subunit; Histone acetyltransferase type B catalytic subunit; Vitamin K-dependent protein C; Cytoskeleton-associated protein 2; and Afamin.

In some embodiments, the biomarkers in the panels herein and/or assessed herein include one or more biomarkers selected from the group consisting of a third panel of protein biomarkers. In some embodiments the third panel comprises: Insulin-like growth factor-binding protein 4; Tumor necrosis factor ligand superfamily member 15; Heat shock 70 kDa protein 1A; Calsyntenin-3; Lamina-associated polypeptide 2, isoforms beta/gamma; C—X—C motif chemokine 13; Troponin T, cardiac muscle; Macrophage-capping protein; CUB domain-containing protein 1; Kallikrein-10; Programmed cell death protein 5; Thioredoxin reductase 1, cytoplasmic; E-selectin; C—X—C motif chemokine 10; Troponin I, fast skeletal muscle; Cadherin-11:Extracellular domain; Zymogen granule membrane protein 16; Interleukin-6 receptor subunit alpha; and Matrix-remodeling-associated protein 8:Extracellular domain.

In some embodiments, cDNAs are generated from nucleic acids encoding the protein biomarkers identified herein. In some embodiments, full-length cDNAs are provided. In some embodiments, cDNAs are provided that span an exon-exon junction. In some embodiments, a panel of one or more cDNAs encode for one or more biomarkers selected from the group consisting of: Table 2; Table 3; Table 4; Table 5; Table 6; Table 7: Table 8; Table 9; Table 10; Table 11; Table 12; and/or Table 13).

In some embodiments, the cDNAs in the panels herein and/or assessed herein encode for one or more biomarkers selected from the group consisting of: the first panel of biomarkers; the second panel of biomarkers; and/or the third panel of biomarkers.

In some embodiments, the levels of one or more biomarkers is selected from the group consisting of: Table 2; Table 3; Table 4; Table 5; Table 6; Table 7; Table 8; Table 9; Table 10; Table 11; Table 12; and/or Table 13 in a sample are quantitated. In some embodiments, an increased level (e.g., relative to a the population average, a reference range, control, or a threshold level) of one or more of the selected protein biomarkers in a biological sample (e.g., blood or plasma sample) from a subject is correlated with increased presence of impaired respiratory health.

In some embodiments, the level (e.g., concentration) of a biomarker is quantified in a sample from the subject using the methods, kits, reagents, etc. herein. In some embodiments, the level of the biomarker is compared to a control value that is representative of a “normal” level for a healthy subject. In some embodiments, the control value is the population average or a value based upon the population average or a reference range. In some embodiments, the difference between the quantified biomarker level and the control value is determined (e.g., quantified biomarker level minus the control value, absolute value of the quantified biomarker level, etc.). In some embodiments, the difference between the quantified biomarker level and the control value is divided by the standard deviation for the population used to determine the control value. In some embodiments, the difference between the quantified biomarker level and the control value divided by the standard deviation is the biomarker score for that given biomarker. In some embodiments, a biomarker score above a threshold value indicates an abnormal level for that protein. In some embodiments, a threshold for an individual biomarker score is 0.5, 1.0, 1.5, 2.0, 2.5, 3.0, 3.5, 4.0, 4.5, 5.0, or more, or ranges or values therebetween.

In some embodiments, a composite metabolite biomarker score is calculated by taking the sum of individual metabolite biomarker scores for a panel of metabolite biomarkers. In some embodiments, a composite metabolite biomarker score is calculated by taking the average of individual metabolite biomarker scores for a panel of metabolite biomarkers. In some embodiments, a composite metabolite biomarker score above a threshold value indicates an abnormal level for that metabolite. In some embodiments, when a composite metabolite biomarker score is an average of individual biomarker scores, a threshold for a composite biomarker score is 0.5, 1.0, 1.5, 2.0, 2.5, 3.0, 3.5, 4.0, 4.5, 5.0, or more, or ranges or values therebetween. In some embodiments, when a composite metabolite biomarker score is a sum of individual biomarker scores, a threshold for a composite biomarker score is the number of biomarkers in the panel multiplied by 0.5, 1.0, 1.5, 2.0, 2.5, 3.0, 3.5, 4.0, 4.5, 5.0, or more, or ranges or values therebetween.

In some embodiments, a subject is determined to have normal lung health (or a high likelihood of having normal lung health) if the composite biomarker score is below a composite threshold value. In some embodiments, the composite threshold value is n*(individual biomarker threshold), wherein n is the number of biomarkers analyzed in the panel and the individual biomarker threshold is 0.5, 0.75, 1.0, 1.25, 1.5, 1.75, 2.0, 2.5, 3.0, 3.5, 4.0, 4.5, 5.0, or more, or ranges or values therebetween.

In some embodiments, a kit is used to analyze a biomarker contained within a biological sample (e.g., blood or plasma sample). In some embodiments, the kit contains all components for quantifying a biomarker contained within a biological sample including at least one (e.g., one or more) biomarker (or a panel of biomarkers) that has a known level (e.g., a known concentration, a known weight, a known amount (e.g., a biomarker standard)). In some embodiments, the kit further contains at least one (e.g., one or more) analytical platforms (e.g., high-throughput platforms, automated platforms, etc.) utilizing a POC device, nuclear magnetic resonance (NMR) spectroscopy, gas chromatography (GC), liquid chromatography (LC), and/or mass spectrometry (MS) or NMR, GC, and/or LC coupled to MS.

### 3. Materials and Methods

The plasma proteomics of 4,410 participants in an older, ever-smoker cohort (Genetic Epidemiology of COPD, COPDGene) and 2,958 participants in a younger population cohort (Coronary Artery Disease Risk in Young Adults, CARDIA) were measured with the SomaLogic SomaScan assays. QIA was measured using a local density histogram method on chest CTs. The associations between proteomics levels and QIA were assessed using multivariable linear regression models adjusted for age, sex, body mass index, smoking status, and study center (Benjamini-Hochberg False Discovery Rate p-value ≤0.05).

### 4. EXAMPLES

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

Study populations. Data was collected from 4,410 participants from a COPDGene study at the five-year follow-up visit (Visit 2) and from 2,958 participants from a CARDIA study at the 25-year follow-up visit (Y25). Complete inspiratory chest CT scans were included and proteomics measurements were available. The study was approved by the institutional review board at all study centers. Study participants provided written, informed consent at each study visit.

COPDGene was a prospective observational study of 10,198 former and current smokers (ever-smokers) with at least a 10-pack-year smoking history, aged 45-80 years at baseline visit, with no prior bronchiectasis or interstitial lung disease (ILD) of Non-Hispanic White or Black self-reported race, from 21 study centers in the US. At Visit 2, participants underwent collection of questionnaires, inspiratory chest CT scans, and proteomic analysis.

CARDIA was a longitudinal study that commenced in 1985 with 5,115 adults from four communities in the US, aged 18-30 years at baseline visit, of self-reported White or Black race. At the Y25 visit, participants underwent collection of questionnaires, inspiratory chest CT scans, and proteomic analysis. Clinical information and questionnaires were collected by standardized assessment.

### Example 2

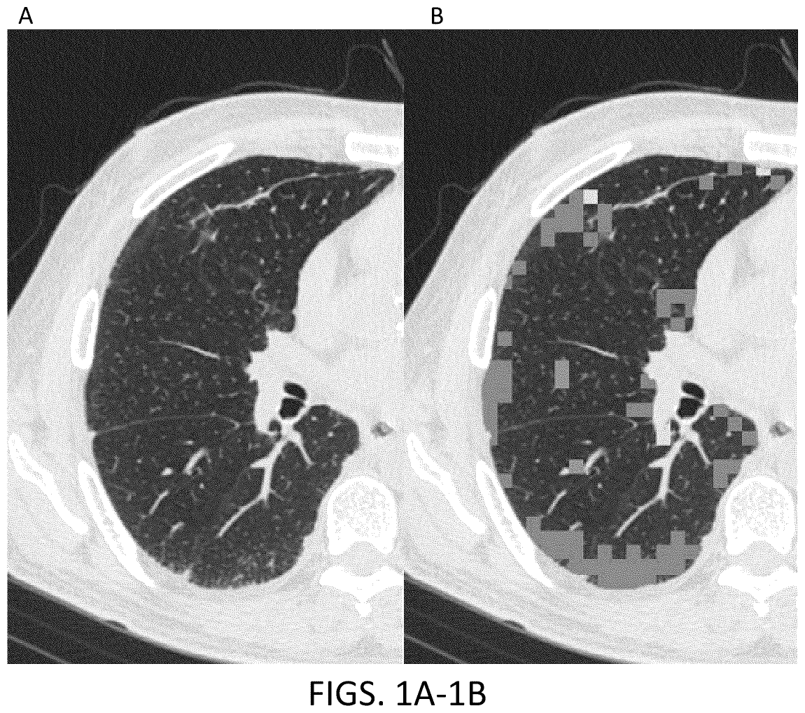

QIA measurements. Inspiratory volumetric CT scans of the chest were obtained using a protocol that was harmonized across centers within each cohort. QIA was measured using a local histogram classifier, as previously described. Briefly, the classifier used a k-nearest neighbors algorithm of the local histogram measurements combined with the distance from the pleural surface to output percentages of CT lung volume occupied by radiologic features. The percentages of reticulation, subpleural lines, ground glass opacities, honeycombing, linear scarring, centrilobular nodules, and other nodularity features were summed to yield QIA percent, which was used as a continuous outcome measure in the study (FIG. 1).

### Example 3

Proteomics measurements. For COPDGene participants, blood was collected in EDTA tubes, and aliquots of plasma were stored at −80° C. For CARDIA participants, venous blood was collected in EDTA tubes, and samples were centrifuged and separated into plasma within 2 hours of collection. All plasma samples were stored in 0.5 ml aliquots at −80° C. until analysis, and 130 μL of plasma was submitted to the SomaScan assay for proteomics analysis.

As previously described, the SomaScan assay used chemically modified single-stranded DNA aptamers called SOMAmers that bind to epitopes on their target protein; the SOMAmer reagents are then hybridized to complementary sequences on a DNA microarray in the SomaScan Assay to yield relative fluorescence units. For COPDGene participants, proteomics identification was performed on plasma samples using the SomaLogic SomaScan version 4.0 (5K) assay, as previously described. The assay used 4,979 SOMAmer aptamers to identify 4,776 unique human proteins. For CARDIA participants, proteomics identification was performed on plasma samples using the SomaLogic SomaScan version 4.1 (7K) assay. The assay used 7,335 SOMAmer aptamers to identify 6,609 unique human proteins. For both assays, standard processes including within plate hybridization, median signal normalization, plate scaling, and calibration were performed independently on all samples. Protein levels were reported in relative fluorescent units (RFU) and natural log-transformed for analysis. To facilitate validation and comparisons between COPDGene and CARDIA, the aptamers included in the analyses were limited to the 4,979 available in the smaller version 4.0 assay used in COPDGene. There was no missing data for any participant included in the analysis.

### Example 4

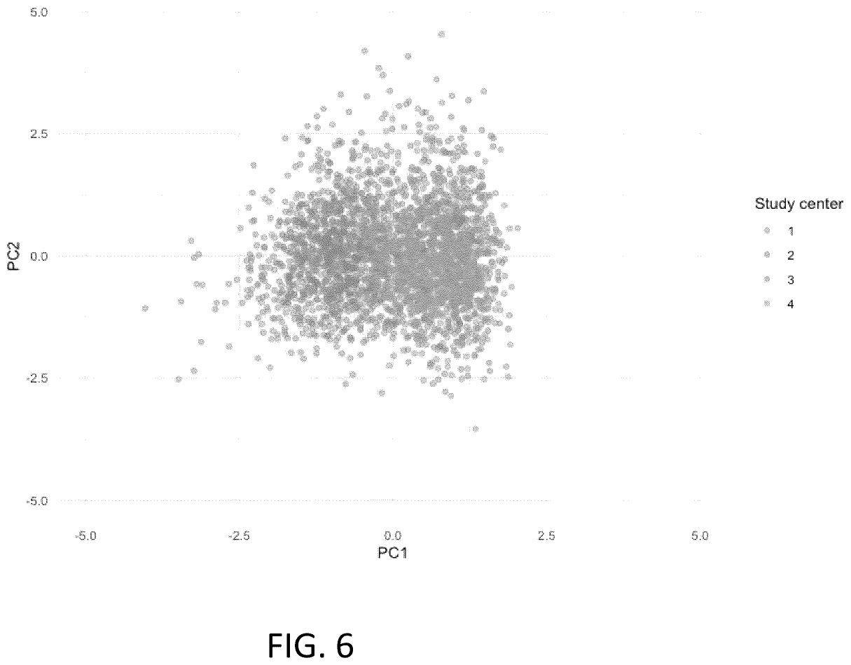

Statistical analyses. To identify the proteins associated with QIA within each cohort, the association between each log-transformed SomaScan protein as a continuous measure and QIA (percent QIA in lung) as a continuous measure was assessed with univariate linear regression, followed by multivariable linear regression adjusted for age, sex, body mass index (BMI), smoking status (current/former/never), and study center as a random effect to adjust for technical variation (20 centers in COPDGene; four centers in CARDIA grouped into two based principle component analysis of the proteins (FIG. 6)). Analyses in COPDGene were additionally adjusted for platelet count and white blood cell count based on prior internal quality control. Multiple testing within each cohort was also adjusted using a Benjamini-Hochberg False Discovery Rate (FDR) p-value of ≤0.05. Analyses were performed using R software (version 4.2.2) implemented using RStudio (version 2022.12.0+353).

### Example 5

Pathway enrichment analysis. Protein Gene Ontology (GO) pathway enrichment analysis of the significant proteins was performed using the Database for Annotation, Visualization and Integrated Discovery (DAVID) web platform. The Entrez Gene identifiers (GeneIDs) linked to each SOMA protein were used and duplicate GeneIDs were excluded. The GeneID list was compared to the Homo Sapiens GO database lists for Molecular Function, Cellular Components, and Biological Process, using Fisher's Exact test and a Benjamini-Hochberg FDR p-value of ≤0.05.

### Example 6

Tissue-specific deconvolution. Tissue-specific deconvolution of the significant proteins using was performed using the Genotype-Tissue Expression (GTEx) database, which is a comprehensive public resource to study tissue-specific gene expression and regulation, from The Human Protein Atlas (HPA). The Entrez GeneIDs linked to each SOMA protein were used and duplicate GeneIDs were excluded before linking them to gene transcript normalized expression levels (nTPM) of 37 tissues from The Human Protein Atlas version 22.0 and Ensembl version 103.38. The gene expression activities of the proteins positively significant for QIA were scored by the R package singscore for each tissue, which were used to rank the 37 tissues.

### Example 7

Results of Examples 1-6. As shown in Table 1, the 4,410 participants in COPDGene had mean age 65.6±8.7 years, were 51.7% female, and 38.4% current and 61.6% former smokers. Participants had mean QIA occupying 5.7±4.6% of the lung, mean percent predicted (pp) forced expiratory volume in 1 second (FEV1) of 77.6±24.4%, and mean pp forced vital capacity (FVC) of 86.6±17.8%. In the CARDIA cohort, the 2,958 participants were younger with mean age 50.2±3.6 years, were similarly split with 56.0% female, and 26.8% current, 22.3% former, and 50.9% never smokers. Participants had less mean QIA occupying the lung of 2.7±5.3%, higher mean ppFEV1 of 93.6±14.4%, and higher mean ppFVC of 96.0±13.8%.

TABLE 1
Baseline characteristics

| | COPDGene | CARDIA | |
|---|---|---|---|
| | N = 4,410 | N = 2,958 | |
| Age, mean ± SD | 65.6 ± 8.7 | 50.2 ± 3.6 | |
| Females, N(%) | 2,280 (51.7) | 1,657 | (56.0) |
| Males, N(%) | 2,130 (48.3) | 1,301 | (44.0) |
| Self-reported White, N(%) | 3,150 (71.4) | 1,585 | (53.6) |
| Self-reported Black, N(%) | 1,260 (28.6) | 1,373 | (46.4) |
| BMI, mean ± SD | 29.0 ± 6.4 | 30.3 ± 7.1 | |
| Current smoker, N(%) | 1,692 (38.4) | 786 | (26.8) |
| Former smoker, N(%) | 2,715 (61.6) | 653 | (22.3) |
| Never smoker, N(%) | — | 1,490 | (50.9) |

| | | |
|---|---|---|
| Pack-years, mean ± SD | 44.0 ± 24.0 | 5.6 ± 10.1 |
| Percent QIA, mean ± SD | 5.7 ± 4.6 | 2.7 ± 5.3 |
| 25^{th} percentile | | |
| Median | | |
| 50^{th} percentile | | |
| Percent quantitative emphysema, | 6.4 ± 13.6 | 1.0 ± 2.1 |
| mean ± SD | | |
| Percent predicted FEV1, mean ± SD | 77.6 ± 24.4 | 93.6 ± 14.4 |
| Percent predicted FVC, mean ± SD | 86.6 ± 17.8 | 96.0 ± 13.8 |

BMI = body mass index,
SD = standard deviation

In univariate regression in COPDGene, there were 2,270 proteins (2,194 unique Entrez GeneIDs) significant for QIA (FDR p≤0.05), with 1,639 proteins positively and 631 negatively associated with QIA (Table 2).

TABLE 2
Univariate regression analysis of QIA in COPDGene

| | | | Percent | Up- | Low- | | |
|---|---|---|---|---|---|---|---|
| | | | QIA per | per | er | | |
| | Entrez | UniProt | ln | 95% | 95% | p- | FDR p- |
| Protein name | gene name | ID | (RFU) | CI | CI | value | value |
| Complement factor H | CFH | P08603 | 6 | 4.42 | 7.58 | 1.16E−13 | 4.33E−12 |
| Complement factor B | CFB | P00751 | 4.65 | 3.71 | 5.59 | 6.11E−22 | 6.76E−20 |
| Lumican | LUM | P51884 | 3.87 | 3.21 | 4.54 | 6.93E−30 | 2.87E−27 |
| C-X-C motif chemokine 16 | CXCL16 | Q9H2A7 | 3.83 | 3.16 | 4.51 | 1.79E−28 | 6.35E−26 |
| Complement factor I | CFI | P05156 | 3.79 | 2.54 | 5.04 | 2.96E−09 | 5.08E−08 |
| EGF-containing fibulin-like extracellular matrix | EFEMP1 | Q12805 | 3.68 | 3.14 | 4.22 | 7.08E−40 | 1.62E−36 |
| protein 1 | | | | | | | |
| Spondin-1 | SPON1 | Q9HCB6 | 3.47 | 2.82 | 4.12 | 1.60E−25 | 2.84E−23 |
| Serine protease HTRA1 | HTRA1 | Q92743 | 3.45 | 2.88 | 4.03 | 6.92E−32 | 4.31E−29 |
| Neuropilin-2 | NRP2 | O60462 | 3.32 | 2.39 | 4.26 | 3.85E−12 | 1.11E−10 |
| Collagen alpha-1(XXVIII) chain | COL28A1 | Q2UY09 | 2.96 | 2.48 | 3.44 | 7.42E−33 | 6.16E−30 |
| Retinoic acid receptor responder protein 2 | RARRES2 | Q99969 | 2.91 | 2.36 | 3.46 | 4.11E−25 | 7.05E−23 |
| N-acetylglucosamine-1-phosphotransferase | GNPTG | Q9UJJ9 | 2.9 | 2.03 | 3.77 | 7.85E−11 | 1.79E−09 |
| subunit gamma | | | | | | | |
| Collagen alpha-3(VI) chain: Bovine pancreatic | COL6A3 | P12111 | 2.86 | 2.35 | 3.36 | 1.91E−28 | 6.35E−26 |
| trypsin inhibitor/Kunitz inhibitor domain, isoform 1 | | | | | | | |
| Cystatin-C | CST3 | P01034 | 2.78 | 2.26 | 3.31 | 6.21E−25 | 1.03E−22 |
| Alpha-1 -microglobulin | AMBP | P02760 | 2.73 | 1.91 | 3.56 | 9.49E−11 | 2.14E−09 |
| Roundabout homolog 2 | ROBO2 | Q9HCK4 | 2.73 | 1.88 | 3.58 | 2.81E−10 | 5.77E−09 |
| Complement C1q tumor necrosis factor-related | CIQTNF1 | Q9BXJ1 | 2.72 | 1.94 | 3.49 | 8.85E−12 | 2.42E−10 |
| protein 1 | | | | | | | |
| Neurofascin | NFASC | O94856 | 2.69 | 2.06 | 3.31 | 5.11E−17 | 2.83E−15 |
| Leukocyte immunoglobulin-like receptor | LILRA5 | A6NI73 | 2.68 | 2.14 | 3.23 | 6.58E−22 | 7.12E−20 |
| subfamily A member 5 | | | | | | | |
| Serine/arginine-rich splicing factor 6 | SRSF6 | Q13247 | 2.65 | 2.14 | 3.15 | 3.50E−24 | 4.84E−22 |
| Beta-2-microglobulin | B2M | P61769 | 2.61 | 2.16 | 3.07 | 8.17E−29 | 3.13E−26 |
| Insulin-like growth factor-binding protein 4 | IGFBP4 | P22692 | 2.59 | 1.99 | 3.19 | 4.08E−17 | 2.34E−15 |
| C-C motif chemokine 18 | CCL18 | P55774 | 2.59 | 2.16 | 3.02 | 5.13E−32 | 3.65E−29 |
| Tumor necrosis factor ligand superfamily | TNFSF15 | O95150 | 2.57 | 2.01 | 3.13 | 3.19E−19 | 2.48E−17 |
| member 15 | | | | | | | |
| Complement component C9 | C9 | P02748 | 2.53 | 1.9 | 3.16 | 3.43E−15 | 1.58E−13 |
| ADAMTS-like protein 2 | ADAMTSL2 | Q86TH1 | 2.5 | 2.06 | 2.95 | 2.63E−28 | 8.20E−26 |
| Lipopolysaccharide-binding protein | LBP | P18428 | 2.41 | 1.55 | 3.26 | 3.34E−08 | 4.46E−07 |
| Complement C2 | C2 | P06681 | 2.41 | 1.67 | 3.15 | 1.79E−10 | 3.85E−09 |
| Membrane cofactor protein | CD46 | P15529 | 2.39 | 1.66 | 3.13 | 1.66E−10 | 3.58E−09 |
| Pigment epithelium-derived factor | SERPINF1 | P36955 | 2.38 | 1.33 | 3.43 | 9.65E−06 | 6.00E−05 |
| C4b-binding protein alpha chain | C4BPA | P04003 | 2.37 | 1.71 | 3.03 | 1.87E−12 | 5.78E−11 |
| Follistatin-related protein 3 | FSTL3 | O95633 | 2.34 | 1.83 | 2.84 | 1.50E−19 | 1.19E−17 |
| Serine/arginine-rich splicing factor 7 | SRSF7 | Q16629 | 2.33 | 1.79 | 2.87 | 5.10E−17 | 2.83E−15 |
| Macrophage mannose receptor 1 | MRC1 | P22897 | 2.33 | 1.74 | 2.93 | 2.51E−14 | 1.03E−12 |
| Junctional adhesion molecule B | JAM2 | P57087 | 2.32 | 1.62 | 3.03 | 1.34E−10 | 2.96E−09 |
| Carbohydrate sulfotransferase 15 | CHST15 | Q7LFX5 | 2.27 | 1.48 | 3.06 | 1.96E−08 | 2.73E−07 |
| Peptidyl-prolyl cis-trans isomerase C | PPIC | P45877 | 2.26 | 1.69 | 2.82 | 4.91E−15 | 2.20E−13 |
| Ribonuclease 4 | RNASE4 | P34096 | 2.24 | 1.52 | 2.95 | 8.86E−10 | 1.70E−08 |
| Coiled-coil domain-containing protein 80 | CCDC80 | Q76M96 | 2.23 | 1.63 | 2.83 | 4.24E−13 | 1.45E−11 |
| Gamma-glutamyl hydrolase | GGH | Q92820 | 2.23 | 1.71 | 2.75 | 5.51E−17 | 3.02E−15 |
| WAP four-disulfide core domain protein 2 | WFDC2 | Q14508 | 2.19 | 1.84 | 2.53 | 4.06E−34 | 4.04E−31 |
| Fibrinogen | FGA FGB | P02679\|P02675\|P02671 | 2.19 | 1.34 | 3.05 | 5.22E−07 | 5.19E−06 |
| | FGG | | | | | | |
| Out at first protein homolog | OAF | Q86UD1 | 2.18 | 1.41 | 2.94 | 2.31E−08 | 3.16E−07 |
| Twisted gastrulation protein homolog 1 | TWSG1 | Q9GZX9 | 2.18 | 1.59 | 2.77 | 4.18E−13 | 1.44E−11 |
| Hepatitis A virus cellular receptor 2 | HAVCR2 | Q8TDQ0 | 2.16 | 1.68 | 2.64 | 2.70E−18 | 1.74E−16 |
| Ganglioside GM2 activator | GM2A | P17900 | 2.15 | 1.68 | 2.62 | 4.37E−19 | 3.16E−17 |
| Microfibrillar-associated protein 2 | MFAP2 | P55001 | 2.13 | 1.48 | 2.79 | 1.66E−10 | 3.58E−09 |
| Gamma-aminobutyric acid receptor-associated | GABARAP | O95166 | 2.11 | 1.68 | 2.54 | 7.65E−22 | 8.11E−20 |
| protein | | | | | | | |
| Retinoblastoma-like protein 2 | RBL2 | Q08999 | 2.08 | 1.64 | 2.51 | 2.76E−20 | 2.45E−18 |
| Sushi, von Willebrand factor type A, EGF and | SVEP1 | Q4LDE5 | 2.06 | 1.69 | 2.43 | 3.82E−27 | 9.25E−25 |
| pentraxin domain-containing protein 1: EGF-like | | | | | | | |
| domains 4-6 | | | | | | | |
| Ephrin type-B receptor 2 | EPHB2 | P29323 | 2.05 | 1.43 | 2.66 | 8.35E−11 | 1.89E−09 |
| Microfibrillar-associated protein 2 | MFAP2 | P55001 | 2.05 | 1.45 | 2.65 | 2.70E−11 | 6.78E−10 |
| Vesicular integral-membrane protein VIP36 | LMAN2 | Q12907 | 2.05 | 1.46 | 2.63 | 7.59E−12 | 2.10E−10 |
| Inactive tyrosine-protein kinase 7 | PTK7 | Q13308 | 2.05 | 1.32 | 2.78 | 3.66E−08 | 4.78E−07 |
| Low-density lipoprotein receptor-related protein | LRP11 | Q86VZ4 | 2.02 | 1.39 | 2.65 | 4.06E−10 | 8.21E−09 |
| 11 | | | | | | | |
| Antileukoproteinase | SLPI | P03973 | 1.97 | 1.31 | 2.63 | 4.79E−09 | 7.90E−08 |
| Gamma-aminobutyric acid receptor-associated | GABARAPL1 | Q9H0R8 | 1.95 | 1.52 | 2.38 | 1.40E−18 | 9.27E−17 |
| protein-like 1 | | | | | | | |
| Allograft inflammatory factor 1-like | AIF1L | Q9BQI0 | 1.95 | 1.38 | 2.51 | 1.92E−11 | 4.92E−10 |
| R-spondin-4 | RSPO4 | Q2I0M5 | 1.95 | 1.43 | 2.47 | 1.87E−13 | 6.76E−12 |
| Sushi, von Willebrand factor type A, EGF and | SVEP1 | Q4LDE5 | 1.94 | 1.56 | 2.33 | 7.34E−23 | 8.91E−21 |
| pentraxin domain-containing protein 1: Sushi 15- | | | | | | | |
| 18 | | | | | | | |
| C-C motif chemokine 14 | CCL14 | Q16627 | 1.92 | 1.6 | 2.24 | 2.91E−31 | 1.45E−28 |
| Galectin-3 | LGALS3 | P17931 | 1.92 | 1.23 | 2.61 | 5.90E−08 | 7.32E−07 |
| Induced myeloid leukemia cell differentiation | MCL1 | Q07820 | 1.89 | 1.46 | 2.32 | 1.10E−17 | 6.66E−16 |
| protein Mcl-1 | | | | | | | |
| Macrophage-capping protein | CAPG | P40121 | 1.86 | 1.49 | 2.23 | 1.20E−22 | 1.43E−20 |
| Folate receptor beta | FOLR2 | P14207 | 1.85 | 1.19 | 2.5 | 2.82E−08 | 3.81E−07 |
| Fibrinogen gamma chain | FGG | P02679 | 1.83 | 1.3 | 2.36 | 1.37E−11 | 3.63E−10 |
| Matrix Gla protein | MGP | P08493 | 1.83 | 1.23 | 2.42 | 1.69E−09 | 3.03E−08 |
| Interleukin-21 receptor | IL21R | Q9HBE5 | 1.82 | 1.12 | 2.51 | 3.25E−07 | 3.53E−06 |
| CUB domain-containing protein 1 | CDCP1 | Q9H5V8 | 1.81 | 1.52 | 2.09 | 2.76E−35 | 3.44E−32 |
| Tumor necrosis factor receptor superfamily | TNFRSF1B | P20333 | 1.81 | 1.42 | 2.21 | 3.98E−19 | 2.92E−17 |
| member 1B | | | | | | | |
| D-dimer | FGA FGB FGG | P02679\|P02675\|P02671 | 1.76 | 1.23 | 2.29 | 9.57E−11 | 2.15E−09 |
| CD166 antigen | ALCAM | Q13740 | 1.76 | 0.94 | 2.57 | 2.43E−05 | 1.27E−04 |
| Fatty acid-binding protein, heart | FABP3 | P05413 | 1.75 | 1.46 | 2.04 | 9.51E−32 | 5.26E−29 |
| Inter-alpha-trypsin inhibitor heavy chain H3 | ITIH3 | Q06033 | 1.75 | 1.3 | 2.19 | 2.68E−14 | 1.09E−12 |
| Trefoil factor 3 | TFF3 | Q07654 | 1.74 | 1.34 | 2.14 | 9.92E−18 | 6.10E−16 |
| Dermatopontin | DPT | Q07507 | 1.74 | 1.22 | 2.26 | 4.49E−11 | 1.08E−09 |
| Serine/threonine-protein phosphatase 4 regulatory | SMEK1 | Q6IN85 | 1.73 | 1.35 | 2.11 | 3.73E−19 | 2.77E−17 |
| subunit 3A | | | | | | | |
| Interleukin-1 receptor antagonist protein | IL1RN | P18510 | 1.73 | 1.32 | 2.14 | 1.82E−16 | 9.66E−15 |
| Galectin-4 | LGALS4 | P56470 | 1.72 | 1.28 | 2.17 | 4.91E−14 | 1.93E−12 |
| Ribonuclease K6 | RNASE6 | Q93091 | 1.71 | 1.34 | 2.08 | 9.68E−20 | 7.77E−18 |
| Small ubiquitin-related modifier 2 | SUMO2 | P61956 | 1.69 | 1.39 | 2 | 3.88E−27 | 9.25E−25 |
| C-C motif chemokine 3 | CCL3 | P10147 | 1.69 | 1.27 | 2.1 | 3.09E−15 | 1.45E−13 |
| Olfactomedin-like protein 3 | OLFML3 | Q9NRN5 | 1.69 | 0.99 | 2.39 | 1.96E−06 | 1.59E−05 |
| E3 ubiquitin-protein ligase SMURF1 | SMURF1 | Q9HCE7 | 1.68 | 0.91 | 2.45 | 1.83E−05 | 1.01E−04 |
| Tumor necrosis factor receptor superfamily | TNFRSF1B | P20333 | 1.68 | 1.29 | 2.07 | 3.92E−17 | 2.27E−15 |
| member 1B | | | | | | | |
| Trefoil factor 3 | TFF3 | Q07654 | 1.68 | 1.22 | 2.13 | 4.35E−13 | 1.47E−11 |
| Copper transport protein ATOX1 | ATOX1 | O00244 | 1.67 | 1.32 | 2.01 | 9.99E−21 | 9.34E−19 |
| Transmembrane emp24 domain-containing | TMED10 | P49755 | 1.67 | 1.25 | 2.09 | 8.44E−15 | 3.62E−13 |
| protein 10 | | | | | | | |
| Calsyntenin-3 | CLSTN3 | Q9BQT9 | 1.66 | 1.26 | 2.06 | 4.59E−16 | 2.38E−14 |
| Endoplasmic reticulum lectin 1 | ERLEC1 | Q96DZ1 | 1.66 | 1.17 | 2.15 | 3.01E−11 | 7.54E−10 |
| Transferrin receptor protein 1: Extracellular | TFRC | P02786 | 1.64 | 1.16 | 2.12 | 1.60E−11 | 4.16E−10 |
| domain | | | | | | | |
| Triggering receptor expressed on myeloid cells 1 | TREM1 | Q9NP99 | 1.64 | 1.24 | 2.03 | 6.39E−16 | 3.25E−14 |
| Vascular endothelial growth factor A | VEGFA | P15692 | 1.62 | 0.91 | 2.34 | 8.85E−06 | 5.58E−05 |
| Phospholipase A2, membrane associated | PLA2G2A | P14555 | 1.62 | 1.31 | 1.92 | 1.67E−24 | 2.45E−22 |
| Butyrophilin subfamily 3 member A3 | BTN3A3 | O00478 | 1.61 | 1.21 | 2.01 | 1.84E−15 | 8.99E−14 |
| B melanoma antigen 2 | BAGE2 | Q86Y30 | 1.61 | 1.22 | 1.99 | 2.43E−16 | 1.27E−14 |
| Pulmonary surfactant-associated protein B | SFTPB | P07988 | 1.59 | 1.29 | 1.89 | 1.59E−24 | 2.41E−22 |
| Nuclease-sensitive element-binding protein 1 | YBX1 | P67809 | 1.58 | 1.24 | 1.91 | 5.53E−20 | 4.66E−18 |
| Phospholipase B-like 1 | PLBD1 | Q6P4A8 | 1.57 | 1.14 | 1.99 | 8.69E−13 | 2.83E−11 |
| Endostatin | COL18A1 | P39060 | 1.56 | 1.04 | 2.08 | 5.72E−09 | 9.18E−08 |
| Thrombospondin-4 | THBS4 | P35443 | 1.56 | 1.04 | 2.09 | 6.02E−09 | 9.61E−08 |
| Thioredoxin domain-containing protein 5 | TXNDC5 | Q8NBS9 | 1.55 | 1.19 | 1.91 | 4.24E−17 | 2.40E−15 |
| Stanniocalcin-1 | STC1 | P52823 | 1.55 | 1.03 | 2.08 | 7.93E−09 | 1.23E−07 |
| Transaldolase | TALDO1 | P37837 | 1.54 | 1.06 | 2.01 | 2.53E−10 | 5.28E−09 |
| Interleukin enhancer-binding factor 3 | ILF3 | Q12906 | 1.54 | 0.98 | 2.11 | 9.79E−08 | 1.17E−06 |
| Set1/Ash2 histone methyltransferase complex | ASH2L | Q9UBL3 | 1.53 | 0.91 | 2.16 | 1.51E−06 | 1.27E−05 |
| subunit ASH2 | | | | | | | |
| Transgelin | TAGLN | Q01995 | 1.52 | 1.11 | 1.92 | 2.18E−13 | 7.80E−12 |
| Growth/differentiation factor 15 | GDF15 | Q99988 | 1.52 | 1.24 | 1.79 | 3.90E−27 | 9.25E−25 |
| Tryptophan--tRNA ligase, cytoplasmic | WARS | P23381 | 1.52 | 1.02 | 2.02 | 2.68E−09 | 4.69E−08 |
| Transmembrane emp24 domain-containing | TMED2 | Q15363 | 1.51 | 0.68 | 2.34 | 3.45E−04 | 1.22E−03 |
| protein 2 | | | | | | | |
| T-cell immunoglobulin and mucin domain- | TIMD4 | Q96H15 | 1.51 | 1.12 | 1.91 | 5.98E−14 | 2.29E−12 |
| containing protein 4 | | | | | | | |
| Neuroblastoma suppressor of tumorigenicity 1 | NBL1 | P41271 | 1.51 | 1.15 | 1.88 | 7.09E−16 | 3.57E−14 |
| Complement C1r subcomponent-like protein | C1RL | Q9NZP8 | 1.51 | 0.7 | 2.32 | 2.81E−04 | 1.02E−03 |
| Hepatoma-derived growth factor | HDGF | P51858 | 1.5 | 1.12 | 1.87 | 4.23E−15 | 1.92E−13 |
| PC4 and SFRS1-interacting protein | PSIP1 | O75475 | 1.5 | 1.12 | 1.87 | 5.47E−15 | 2.43E−13 |
| Prefoldin subunit 2 | PFDN2 | Q9UHV9 | 1.49 | 1 | 1.98 | 2.63E−09 | 4.62E−08 |
| C-C motif chemokine 15 | CCL15 | Q16663 | 1.48 | 1.06 | 1.89 | 2.87E−12 | 8.49E−11 |
| Acyl-CoA-binding domain-containing protein 6 | ACBD6 | Q9BR61 | 1.46 | 0.98 | 1.94 | 2.46E−09 | 4.34E−08 |
| Prenylcysteine oxidase-like | PCYOX1L | Q8NBM8 | 1.46 | 0.95 | 1.97 | 1.84E−08 | 2.58E−07 |
| SPARC-related modular calcium-binding protein | SMOC2 | Q9H3U7 | 1.44 | 0.9 | 1.98 | 1.50E−07 | 1.74E−06 |
| 2 | | | | | | | |
| Stathmin | STMN1 | P16949 | 1.44 | 1 | 1.89 | 2.43E−10 | 5.10E−09 |
| Stromal cell-derived factor 2-like protein 1 | SDF2L1 | Q9HCN8 | 1.44 | 0.87 | 2.01 | 6.94E−07 | 6.56E−06 |
| High mobility group protein 20A | HMG20A | Q9NP66 | 1.43 | 0.81 | 2.04 | 5.45E−06 | 3.77E−05 |
| Serpin B8 | SERPINB8 | P50452 | 1.43 | 0.87 | 1.99 | 5.18E−07 | 5.18E−06 |
| Signal-regulatory protein beta-2 | SIRPB2 | Q5JXA9 | 1.43 | 0.98 | 1.88 | 6.33E−10 | 1.26E−08 |
| Phosphatidylinositol transfer protein alpha | PITPNA | Q00169 | 1.43 | 0.66 | 2.21 | 2.96E−04 | 1.06E−03 |
| isoform | | | | | | | |
| Fibulin-5 | FBLN5 | Q9UBX5 | 1.42 | 0.92 | 1.93 | 2.92E−08 | 3.91E−07 |
| Rho-related GTP-binding protein RhoB | RHOB | P62745 | 1.42 | 0.85 | 1.99 | 9.25E−07 | 8.29E−06 |
| NADH-cytochrome b5 reductase 1 | CYB5R1 | Q9UHQ9 | 1.42 | 0.77 | 2.07 | 1.84E−05 | 1.02E−04 |
| Proteasome subunit beta type-3 | PSMB3 | P49720 | 1.42 | 0.8 | 2.03 | 6.25E−06 | 4.20E−05 |
| Plexin-D1 | PLXND1 | Q9Y4D7 | 1.42 | 0.83 | 2.01 | 2.46E−06 | 1.92E−05 |
| IGF-like family receptor 1 | IGFLR1 | Q9H665 | 1.42 | 1.1 | 1.75 | 2.96E−17 | 1.74E−15 |
| Core-binding factor subunit beta | CBFB | Q13951 | 1.41 | 0.59 | 2.23 | 7.20E−04 | 2.31E−03 |
| Complement C5 | C5 | P01031 | 1.41 | 0.35 | 2.46 | 8.79E−03 | 2.16E−02 |
| Beta-galactoside alpha-2,6-sialyltransferase 1 | ST6GAL1 | P15907 | 1.4 | 0.79 | 2.02 | 8.09E−06 | 5.20E−05 |
| Peroxidasin homolog | PXDN | Q92626 | 1.39 | 1.12 | 1.66 | 2.00E−23 | 2.48E−21 |
| Leucine-rich repeats and immunoglobulin-like | LRIG1 | Q96JA1 | 1.39 | 0.79 | 1.99 | 5.66E−06 | 3.89E−05 |
| domains protein 1 | | | | | | | |
| Thrombospondin-2 | THBS2 | P35442 | 1.39 | 1.13 | 1.66 | 7.29E−25 | 1.17E−22 |
| RNA-binding protein EWS | EWSR1 | Q01844 | 1.38 | 0.93 | 1.83 | 2.77E−09 | 4.80E−08 |
| Marginal zone B- and B1-cell-specific protein | PACAP | Q8WU39 | 1.38 | 1.07 | 1.69 | 5.95E−18 | 3.70E−16 |
| Lysozyme C | LYZ | P61626 | 1.38 | 0.97 | 1.8 | 5.62E−11 | 1.33E−09 |
| Granulins | GRN | P28799 | 1.38 | 0.65 | 2.1 | 1.88E−04 | 7.22E−04 |
| R-spondin-3 | RSPO3 | Q9BXY4 | 1.38 | 0.78 | 1.98 | 7.26E−06 | 4.76E−05 |
| WAP four-disulfide core domain protein 1 | WFDC1 | Q9HC57 | 1.38 | 0.94 | 1.82 | 7.28E−10 | 1.42E−08 |
| Chordin-like protein 1 | CHRDL1 | Q9BU40 | 1.37 | 0.9 | 1.85 | 1.17E−08 | 1.73E−07 |
| Fibroblast growth factor-binding protein 3 | FGFBP3 | Q8TAT2 | 1.36 | 0.78 | 1.93 | 3.80E−06 | 2.77E−05 |
| HLA class I histocompatibility antigen, alpha | HLA-G | P17693 | 1.36 | 0.49 | 2.23 | 2.11E−03 | 6.00E−03 |
| chain G | | | | | | | |
| Ephrin-A4 | EFNA4 | P52798 | 1.36 | 0.8 | 1.92 | 1.76E−06 | 1.44E−05 |
| Low affinity immunoglobulin gamma Fc region | FCGR3B | O75015 | 1.36 | 0.95 | 1.78 | 1.39E−10 | 3.05E−09 |
| receptor III-B | | | | | | | |
| C-X-C motif chemokine 13 | CXCL13 | O43927 | 1.36 | 1.02 | 1.7 | 3.11E−15 | 1.45E−13 |
| Nectin-2 | PVRL2 | Q92692 | 1.36 | 0.62 | 2.1 | 3.01E−04 | 1.08E−03 |
| Angiopoietin-2 | ANGPT2 | O15123 | 1.35 | 1.02 | 1.67 | 1.06E−15 | 5.29E−14 |
| Delta-like protein 1 | DLL1 | O00548 | 1.35 | 0.78 | 1.92 | 3.25E−06 | 2.42E−05 |
| Tenascin | TNC | P24821 | 1.35 | 0.88 | 1.83 | 2.66E−08 | 3.59E−07 |
| Myosin light chain 6B | MYL6B | P14649 | 1.34 | 0.89 | 1.78 | 5.71E−09 | 9.18E−08 |
| WD repeat-containing protein 5 | WDR5 | P61964 | 1.34 | 0.76 | 1.91 | 5.90E−06 | 3.99E−05 |
| Insulin-like growth factor-binding protein 7 | IGFBP7 | Q16270 | 1.34 | 0.6 | 2.08 | 3.69E−04 | 1.29E−03 |
| Tenascin | TNC | P24821 | 1.34 | 0.82 | 1.85 | 4.59E−07 | 4.71E−06 |
| Fatty acid-binding protein, adipocyte | FABP4 | P15090 | 1.33 | 1.07 | 1.58 | 3.12E−24 | 4.44E−22 |
| C-C motif chemokine 21 | CCL21 | O00585 | 1.33 | 0.9 | 1.75 | 1.35E−09 | 2.46E−08 |
| Protein S100-A11 | S100A11 | P31949 | 1.32 | 0.77 | 1.88 | 3.32E−06 | 2.46E−05 |
| Testis-expressed sequence 29 protein | TEX29 | Q8N6K0 | 1.31 | 0.78 | 1.84 | 1.13E−06 | 9.93E−06 |
| Alpha-parvin | PARVA | Q9NVD7 | 1.31 | 0.85 | 1.76 | 1.78E−08 | 2.51E−07 |
| Protein dpy-30 homolog | DPY30 | Q9C005 | 1.31 | 0.98 | 1.64 | 9.00E−15 | 3.83E−13 |
| Matrilysin | MMP7 | P09237 | 1.31 | 1.04 | 1.58 | 8.31E−22 | 8.62E−20 |
| Protein ABHD14A | ABHD14A | Q9BUJ0 | 1.31 | 0.99 | 1.64 | 1.89E−15 | 9.13E−14 |
| A disintegrin and metalloproteinase with | ADAMTS6 | Q9UKP5 | 1.31 | 0.86 | 1.75 | 8.05E−09 | 1.24E−07 |
| thrombospondin motifs 6 | | | | | | | |
| Septin-11 | 43719 | Q9NVA2 | 1.3 | 0.83 | 1.78 | 8.60E−08 | 1.04E−06 |
| Alpha-1B-glycoprotein | A1BG | P04217 | 1.3 | 0.48 | 2.12 | 1.88E−03 | 5.41E−03 |
| Ectonucleotide | ENPP2 | Q13822 | 1.3 | 0.89 | 1.71 | 7.81E−10 | 1.51E−08 |
| pyrophosphatase/phosphodiesterase family | | | | | | | |
| member 2 | | | | | | | |
| Heat shock cognate 71 kDa protein | HSPA8 | P11142 | 1.29 | 0.78 | 1.79 | 7.70E−07 | 7.11E−06 |
| Follistatin-related protein 1 | FSTL1 | Q12841 | 1.28 | 0.29 | 2.27 | 1.14E−02 | 2.71E−02 |
| Steroid receptor RNA activator 1 | SRA1 | Q9HD15 | 1.28 | 0.96 | 1.6 | 5.64E−15 | 2.49E−13 |
| Prostaglandin-H2 D-isomerase | PTGDS | P41222 | 1.27 | 0.84 | 1.69 | 4.25E−09 | 7.14E−08 |
| Ran-binding protein 3 | RANBP3 | Q9H6Z4 | 1.27 | 0.85 | 1.69 | 3.07E−09 | 5.26E−08 |
| Calcium-binding and coiled-coil domain- | CALCOCO2 | Q13137 | 1.26 | 0.49 | 2.03 | 1.36E−03 | 4.05E−03 |
| containing protein 2 | | | | | | | |
| Desmocollin-2 | DSC2 | Q02487 | 1.26 | 0.74 | 1.79 | 2.44E−06 | 1.90E−05 |
| Plastin-2 | LCP1 | P13796 | 1.26 | 0.49 | 2.02 | 1.31E−03 | 3.93E−03 |
| Alpha-1-antitrypsin | SERPINA1 | P01009 | 1.26 | 0.74 | 1.77 | 1.67E−06 | 1.38E−05 |
| Brain natriuretic peptide 32 | NPPB | P16860 | 1.26 | 0.56 | 1.96 | 4.52E−04 | 1.54E−03 |
| Interleukin-18 | IL18 | Q14116 | 1.26 | 0.87 | 1.65 | 3.30E−10 | 6.70E−09 |
| Fibulin-1 | FBLN1 | P23142 | 1.26 | 0.44 | 2.08 | 2.59E−03 | 7.22E−03 |
| CUB and sushi domain-containing protein 2 | CSMD2 | Q7Z408 | 1.26 | 0.76 | 1.77 | 9.91E−07 | 8.79E−06 |
| Alpha-1-antichymotrypsin complex | SERPINA3 | P07288\|P01011 | 1.25 | 0.85 | 1.65 | 1.07E−09 | 2.00E−08 |
| Transferrin receptor protein 1: Cytoplasmic | TFRC | P02786 | 1.25 | 0.88 | 1.62 | 5.04E−11 | 1.21E−09 |
| domain | | | | | | | |
| Tenascin | TNC | P24821 | 1.25 | 0.76 | 1.74 | 7.01E−07 | 6.57E−06 |
| Collagen alpha-1(XV) chain | COL15A1 | P39059 | 1.25 | 0.67 | 1.83 | 2.43E−05 | 1.27E−04 |
| Nucleobindin-1 | NUCB1 | Q02818 | 1.24 | 0.58 | 1.91 | 2.46E−04 | 9.14E−04 |
| Hepatocyte growth factor | HGF | P14210 | 1.24 | 0.84 | 1.65 | 1.93E−09 | 3.43E−08 |
| E-selectin | SELE | P16581 | 1.24 | 0.89 | 1.58 | 2.75E−12 | 8.19E−11 |
| Atrial natriuretic factor | NPPA | P01160 | 1.24 | 0.81 | 1.67 | 1.64E−08 | 2.36E−07 |
| Oncoprotein-induced transcript 3 protein | OIT3 | Q8WWZ8 | 1.24 | 0.81 | 1.67 | 1.35E−08 | 1.99E−07 |
| Limbic system-associated membrane protein | LSAMP | Q13449 | 1.23 | 0.48 | 1.99 | 1.31E−03 | 3.93E−03 |
| Angiogenin | ANG | P03950 | 1.23 | 0.71 | 1.75 | 3.79E−06 | 2.76E−05 |
| CMRF35-like molecule 6 | CD300C | Q08708 | 1.22 | 0.7 | 1.74 | 4.25E−06 | 3.04E−05 |
| Killer cell immunoglobulin-like receptor 2DL4 | KIR2DL4 | Q99706 | 1.22 | 0.69 | 1.74 | 5.64E−06 | 3.88E−05 |
| C-X-C motif chemokine 14 | CXCL14 | O95715 | 1.22 | 0.7 | 1.73 | 3.16E−06 | 2.35E−05 |
| Interleukin-9 | IL9 | P15248 | 1.22 | 0.65 | 1.79 | 3.01E−05 | 1.52E−04 |
| NTF2-related export protein 1 | NXT1 | Q9UKK6 | 1.22 | 0.74 | 1.7 | 7.41E−07 | 6.87E−06 |
| Rho GTPase-activating protein 1: Rho-GTPase | ARHGAP1 | Q07960 | 1.21 | 0.81 | 1.61 | 4.30E−09 | 7.19E−08 |
| activating protein domain | | | | | | | |
| Thiamin pyrophosphokinase 1 | TPK1 | Q9H3S4 | 1.21 | 0.63 | 1.79 | 4.03E−05 | 1.95E−04 |
| Golgi membrane protein 1 | GOLM1 | Q8NBJ4 | 1.21 | 0.86 | 1.55 | 4.65E−12 | 1.33E−10 |
| Regulator of G-protein signaling 1 | RGS1 | Q08116 | 1.2 | 0.59 | 1.82 | 1.25E−04 | 5.08E−04 |
| Leucine-rich repeat transmembrane protein | FLRT1 | Q9NZU1 | 1.2 | 0.73 | 1.67 | 4.72E−07 | 4.84E−06 |
| FLRT1 | | | | | | | |
| Macrophage scavenger receptor types I and | MSR1 | P21757 | 1.19 | 0.89 | 1.5 | 2.48E−14 | 1.03E−12 |
| II: Extracellular domain | | | | | | | |
| Dual specificity mitogen-activated protein kinase | MAP2K2 | P36507 | 1.19 | 0.8 | 1.58 | 2.87E−09 | 4.97E−08 |
| kinase 2 | | | | | | | |
| Histidine triad nucleotide-binding protein 1 | HINT1 | P49773 | 1.19 | 0.82 | 1.56 | 2.98E−10 | 6.10E−09 |
| Glutathione S-transferase omega-1 | GSTO1 | P78417 | 1.18 | 0.57 | 1.78 | 1.50E−04 | 5.92E−04 |
| Axin-2 | AXIN2 | Q9Y2T1 | 1.18 | 0.67 | 1.68 | 5.68E−06 | 3.90E−05 |
| Ubiquitin-conjugating enzyme E2 G2 | UBE2G2 | P60604 | 1.18 | 0.65 | 1.7 | 1.03E−05 | 6.30E−05 |
| Neural proliferation differentiation and control | NPDC1 | Q9NQX5 | 1.17 | 0.68 | 1.66 | 2.94E−06 | 2.22E−05 |
| protein 1 | | | | | | | |
| RELT-like protein 1 | RELL1 | Q8IUW5 | 1.17 | 0.67 | 1.67 | 3.81E−06 | 2.77E−05 |
| Uncharacterized protein KIAA0040 | KIAA0040 | Q15053 | 1.17 | 0.6 | 1.73 | 4.92E−05 | 2.31E−04 |
| Ribonuclease pancreatic | RNASE1 | P07998 | 1.17 | 0.94 | 1.4 | 1.40E−23 | 1.84E−21 |
| Tyrosine-protein kinase transmembrane receptor | ROR2 | Q01974 | 1.17 | 0.78 | 1.55 | 3.54E−09 | 5.99E−08 |
| ROR2 | | | | | | | |
| Lamina-associated polypeptide 2, isoforms | TMPO | P42167 | 1.17 | 0.86 | 1.48 | 2.35E−13 | 8.28E−12 |
| beta/gamma | | | | | | | |
| Complement C1s subcomponent | C1S | P09871 | 1.17 | 0.25 | 2.08 | 1.27E−02 | 3.00E−02 |
| DnaJ homolog subfamily B member 6 | DNAJB6 | O75190 | 1.16 | 0.77 | 1.56 | 8.85E−09 | 1.35E−07 |
| Trafficking protein particle complex subunit 3 | TRAPPC3 | O43617 | 1.16 | 0.77 | 1.55 | 4.26E−09 | 7.14E−08 |
| Macrophage metalloelastase | MMP12 | P39900 | 1.16 | 0.89 | 1.44 | 1.75E−16 | 9.36E−15 |
| Neuropilin-1 | NRP1 | O14786 | 1.16 | 0.53 | 1.78 | 2.93E−04 | 1.06E−03 |
| Y-box-binding protein 2 | YBX2 | Q9Y2T7 | 1.16 | 0.8 | 1.53 | 4.37E−10 | 8.81E−09 |
| Programmed cell death protein 5 | PDCD5 | O14737 | 1.15 | 0.86 | 1.44 | 1.64E−14 | 6.91E−13 |
| C-C motif chemokine 22 | CCL22 | O00626 | 1.15 | 0.8 | 1.49 | 7.02E−11 | 1.64E−09 |
| Ephrin type-A receptor 2 | EPHA2 | P29317 | 1.15 | 0.72 | 1.58 | 2.10E−07 | 2.37E−06 |
| Polypeptide N-acetylgalactosaminyltransferase 1 | GALNT1 | Q10472 | 1.15 | 0.41 | 1.89 | 2.26E−03 | 6.39E−03 |
| Hepatoma-derived growth factor | HDGF | P51858 | 1.15 | 0.81 | 1.49 | 3.85E−11 | 9.44E−10 |
| Immunoglobulin A | IGHA1 IGHA2 | P01877\|P01876 | 1.14 | 0.84 | 1.45 | 2.83E−13 | 9.94E−12 |
| Complement C3d fragment | C3 | P01024 | 1.14 | 0.71 | 1.56 | 1.97E−07 | 2.24E−06 |
| Protein CYR61 | CYR61 | O00622 | 1.14 | 0.68 | 1.6 | 1.13E−06 | 9.93E−06 |
| Intercellular adhesion molecule 5 | ICAM5 | Q9UMFO | 1.14 | 0.71 | 1.58 | 2.66E−07 | 2.95E−06 |
| Mitogen-activated protein kinase 12 | MAPK12 | P53778 | 1.13 | 0.62 | 1.64 | 1.62E−05 | 9.10E−05 |
| Protein disulfide-isomerase TMX3 | TMX3 | Q96JJ7 | 1.13 | 0.69 | 1.56 | 3.65E−07 | 3.91E−06 |
| Dolichyl-diphosphooligosaccharide--protein | RPN1 | P04843 | 1.12 | 0.76 | 1.48 | 1.15E−09 | 2.12E−08 |
| glycosyltransferase subunit 1: Cytoplasmic | | | | | | | |
| domain | | | | | | | |
| Annexin A2 | ANXA2 | P07355 | 1.12 | 0.71 | 1.52 | 6.72E−08 | 8.25E−07 |
| Chloride intracellular channel protein 2 | CLIC2 | O15247 | 1.12 | 0.69 | 1.55 | 4.49E−07 | 4.64E−06 |
| Dual specificity mitogen-activated protein kinase | MAP2K1 | Q02750 | 1.12 | 0.67 | 1.57 | 1.08E−06 | 9.58E−06 |
| kinase 1 | | | | | | | |
| Cryptic protein | CFC1 | POCG37 | 1.12 | 0.58 | 1.65 | 4.28E−05 | 2.06E−04 |
| Elafin | PI3 | P19957 | 1.11 | 0.79 | 1.42 | 6.63E−12 | 1.85E−10 |
| Protein FAM189A2 | FAM189A2 | Q15884 | 1.11 | 0.54 | 1.68 | 1.34E−04 | 5.38E−04 |
| Spondin-2 | SPON2 | Q9BUD6 | 1.11 | 0.74 | 1.48 | 4.17E−09 | 7.03E−08 |
| Plexin-B2 | PLXNB2 | O15031 | 1.11 | 0.38 | 1.85 | 3.07E−03 | 8.43E−03 |
| Protocadherin alpha-C2 | PCDHAC2 | Q9Y5I4 | 1.11 | 0.53 | 1.69 | 1.72E−04 | 6.67E−04 |
| Procollagen C-endopeptidase enhancer 1 | PCOLCE | Q15113 | 1.1 | 0.65 | 1.55 | 1.78E−06 | 1.45E−05 |
| Natriuretic peptides B | NPPB | P16860 | 1.1 | 0.72 | 1.48 | 1.68E−08 | 2.41E−07 |
| Tumor necrosis factor receptor superfamily | TNFRSF19 | Q9NS68 | 1.1 | 0.65 | 1.54 | 1.56E−06 | 1.30E−05 |
| member 19 | | | | | | | |
| Monocyte differentiation antigen CD14 | CD14 | P08571 | 1.1 | 0.41 | 1.78 | 1.85E−03 | 5.34E−03 |
| UPF0577 protein KIAA1324: Cytoplasmic | KIAA1324 | Q6UXG2 | 1.1 | 0.61 | 1.58 | 1.15E−05 | 6.90E−05 |
| domain | | | | | | | |
| Transformer-2 protein homolog beta | TRA2B | P62995 | 1.09 | 0.75 | 1.42 | 2.44E−10 | 5.10E−09 |
| Switch-associated protein 70 | SWAP70 | Q9UH65 | 1.09 | 0.53 | 1.66 | 1.59E−04 | 6.24E−04 |
| Vascular endothelial growth factor C | VEGFC | P49767 | 1.09 | 0.68 | 1.5 | 2.13E−07 | 2.40E−06 |
| Holo-Transcobalamin-2 | TCN2 | P20062 | 1.09 | 0.53 | 1.65 | 1.46E−04 | 5.80E−04 |
| Heat shock 70 kDa protein 1A | FURIN | P0DMV8 | 1.09 | 0.38 | 1.8 | 2.52E−03 | 7.05E−03 |
| Nuclear protein MDM1 | MDM1 | Q8TC05 | 1.09 | 0.49 | 1.69 | 3.51E−04 | 1.23E−03 |
| Heat shock 70 kDa protein 1A | HSPA1A | P0DMV8 | 1.08 | 0.42 | 1.74 | 1.41E−03 | 4.18E−03 |
| Intercellular adhesion molecule 1 | ICAM1 | P05362 | 1.08 | 0.74 | 1.42 | 5.48E−10 | 1.10E−08 |
| Heparanase | HPSE | Q9Y251 | 1.08 | 0.71 | 1.45 | 1.48E−08 | 2.15E−07 |
| Egl nine homolog 1 | EGLN1 | Q9GZT9 | 1.08 | 0.61 | 1.55 | 5.69E−06 | 3.90E−05 |
| Carbonyl reductase [NADPH] 1 | CBR1 | P16152 | 1.07 | 0.62 | 1.52 | 2.54E−06 | 1.96E−05 |
| R-spondin-3 | RSPO3 | Q9BXY4 | 1.07 | 0.53 | 1.61 | 1.09E−04 | 4.55E−04 |
| Ras-related protein Rab-3B | RAB3B | P20337 | 1.07 | 0.68 | 1.46 | 7.99E−08 | 9.68E−07 |
| Tumor necrosis factor receptor superfamily | TNFRSF1A | P19438 | 1.07 | 0.71 | 1.44 | 7.01E−09 | 1.10E−07 |
| member 1A | | | | | | | |
| Fibrinogen-like protein 1 | FGL1 | Q08830 | 1.07 | 0.79 | 1.36 | 1.73E−13 | 6.32E−12 |
| BH3-interacting domain death agonist | BID | P55957 | 1.07 | 0.75 | 1.39 | 6.41E−11 | 1.51E−09 |
| Stathmin-3 | STMN3 | Q9NZ72 | 1.07 | 0.66 | 1.49 | 4.07E−07 | 4.26E−06 |
| Progesterone receptor | PGR | P06401 | 1.06 | 0.76 | 1.37 | 7.03E−12 | 1.96E−10 |
| Glutathione S-transferase P | GSTP1 | P09211 | 1.06 | 0.69 | 1.43 | 2.58E−08 | 3.50E−07 |
| Adenosine deaminase CECR1 | CECR1 | Q9NZK5 | 1.06 | 0.75 | 1.37 | 3.12E−11 | 7.76E−10 |
| Eukaryotic translation initiation factor 5 | EIF5 | P55010 | 1.05 | 0.75 | 1.36 | 1.82E−11 | 4.69E−10 |
| C-X-C motif chemokine 10 | CXCL10 | P02778 | 1.05 | 0.73 | 1.37 | 1.58E−10 | 3.44E−09 |
| C-C motif chemokine 7 | CCL7 | P80098 | 1.05 | 0.46 | 1.64 | 5.07E−04 | 1.71E−03 |
| Cyclin-dependent kinases regulatory subunit 1 | CKS1B | P61024 | 1.04 | 0.5 | 1.59 | 1.90E−04 | 7.27E−04 |
| Thyroxine-binding globulin | SERPINA7 | P05543 | 1.04 | 0.19 | 1.88 | 1.60E−02 | 3.66E−02 |
| Fibroblast growth factor 23 | FGF23 | Q9GZV9 | 1.04 | 0.7 | 1.38 | 2.60E−09 | 4.57E−08 |
| Tumor necrosis factor receptor superfamily | TNFRSF11B | O00300 | 1.04 | 0.66 | 1.41 | 6.11E−08 | 7.57E−07 |
| member 11B | | | | | | | |
| Thrombospondin-3 | THBS3 | P49746 | 1.04 | 0.57 | 1.5 | 1.19E−05 | 7.05E−05 |
| Leukocyte-specific transcript 1 protein | LST1 | O00453 | 1.04 | 0.67 | 1.42 | 4.83E−08 | 6.18E−07 |
| Signaling lymphocytic activation molecule | SLAMF1 | Q13291 | 1.03 | 0.6 | 1.47 | 2.97E−06 | 2.24E−05 |
| Ephrin-B1 | EFNB1 | P98172 | 1.02 | 0.57 | 1.48 | 1.03E−05 | 6.30E−05 |
| Macrophage colony-stimulating factor 1 receptor | CSF1R | P07333 | 1.02 | 0.58 | 1.46 | 4.96E−06 | 3.45E−05 |
| Uridine phosphorylase 1 | UPP1 | Q16831 | 1.02 | 0.6 | 1.44 | 2.26E−06 | 1.80E−05 |
| Erythropoietin | EPO | P01588 | 1.02 | 0.68 | 1.35 | 2.73E−09 | 4.76E−08 |
| B-cell differentiation antigen CD72 | CD72 | P21854 | 1.02 | 0.45 | 1.59 | 4.55E−04 | 1.55E−03 |
| Protein Red | IK | Q13123 | 1.02 | 0.19 | 1.84 | 1.62E−02 | 3.70E−02 |
| Suppressor of cytokine signaling 3 | SOCS3 | O14543 | 1.01 | 0.5 | 1.53 | 1.16E−04 | 4.79E−04 |
| Secreted and transmembrane protein 1 | SECTM1 | Q8WVN6 | 1.01 | 0.39 | 1.63 | 1.41E−03 | 4.18E−03 |
| SPARC-related modular calcium-binding protein | SMOC1 | Q9H4F8 | 1.01 | 0.38 | 1.64 | 1.78E−03 | 5.16E−03 |
| 1 | | | | | | | |
| ADP-sugar pyrophosphatase | NUDT5 | Q9UKK9 | 1.01 | 0.49 | 1.53 | 1.33E−04 | 5.36E−04 |
| Metallophosphoesterase MPPED2 | MPPED2 | Q15777 | 1.01 | 0.27 | 1.76 | 7.97E−03 | 1.98E−02 |
| Complement C3 | C3 | P01024 | 1.01 | 0.47 | 1.55 | 2.72E−04 | 9.98E−04 |
| Macrophage colony-stimulating factor 1 | CSF1 | P09603 | 1.01 | 0.51 | 1.51 | 7.01E−05 | 3.11E−04 |
| Semaphorin-4D | SEMA4D | Q92854 | 1.01 | 0.55 | 1.48 | 2.15E−05 | 1.15E−04 |
| Anaphase-promoting complex subunit 10 | ANAPC10 | Q9UM13 | 1 | 0.3 | 1.7 | 5.27E−03 | 1.38E−02 |
| Septin-6 | 43714 | Q14141 | 1 | 0.6 | 1.39 | 7.28E−07 | 6.76E−06 |
| Microtubule-associated proteins 1A/1B light | MAP1LC3A | Q9H492 | 1 | 0.69 | 1.3 | 1.58E−10 | 3.44E−09 |
| chain 3A | | | | | | | |
| 60S ribosomal protein L12 | RPL12 | P30050 | 1 | 0.74 | 1.26 | 5.85E−14 | 2.26E−12 |
| CD226 antigen | CD226 | Q15762 | 1 | 0.46 | 1.53 | 2.69E−04 | 9.86E−04 |
| Reticulon-4 receptor | RTN4R | Q9BZR6 | 1 | 0.53 | 1.46 | 2.69E−05 | 1.39E−04 |
| Glucosidase 2 subunit beta | PRKCSH | P14314 | 1 | 0.46 | 1.54 | 2.62E−04 | 9.62E−04 |
| Noelin | OLFM1 | Q99784 | 1 | 0.54 | 1.46 | 2.23E−05 | 1.19E−04 |
| Receptor-interacting serine/threonine-protein | RIPK2 | O43353 | 1 | 0.56 | 1.44 | 7.92E−06 | 5.11E−05 |
| kinase 2 | | | | | | | |
| Fructose-2,6-bisphosphatase TIGAR | TIGAR | Q9NQ88 | 0.99 | 0.5 | 1.49 | 9.16E−05 | 3.91E−04 |
| Transcription elongation factor A protein 2 | TCEA2 | Q15560 | 0.99 | 0.41 | 1.56 | 7.29E−04 | 2.34E−03 |
| Mucin-16 | MUC16 | Q8WXI7 | 0.99 | 0.61 | 1.38 | 4.08E−07 | 4.26E−06 |
| Pulmonary surfactant-associated protein D | SFTPD | P35247 | 0.99 | 0.75 | 1.22 | 5.10E−16 | 2.62E−14 |
| C-type mannose receptor 2 | MRC2 | Q9UBG0 | 0.99 | 0.39 | 1.59 | 1.26E−03 | 3.82E−03 |
| Bromodomain-containing protein 4 | BRD4 | O60885 | 0.98 | 0.48 | 1.49 | 1.42E−04 | 5.66E−04 |
| 5-hydroxytryptamine receptor 2A | HTR2A | P28223 | 0.98 | 0.44 | 1.53 | 4.39E−04 | 1.50E−03 |
| Cullin-associated NEDD8-dissociated protein 1 | CAND1 | Q86VP6 | 0.98 | 0.63 | 1.33 | 3.84E−08 | 5.00E−07 |
| BPI fold-containing family B member 1 | BPIFB1 | Q8TDL5 | 0.98 | 0.72 | 1.25 | 2.23E−13 | 7.91E−12 |
| Valacyclovir hydrolase | BPHL | Q86WA6 | 0.98 | 0.42 | 1.53 | 5.77E−04 | 1.91E−03 |
| Serum amyloid A-2 protein | SAA2 | P0DJI9 | 0.98 | 0.76 | 1.2 | 4.32E−18 | 2.72E−16 |
| Tumor necrosis factor receptor superfamily | TNFRSF17 | Q02223 | 0.98 | 0.51 | 1.44 | 4.42E−05 | 2.11E−04 |
| member 17 | | | | | | | |
| DnaJ homolog subfamily A member 4 | DNAJA4 | Q8WW22 | 0.98 | 0.59 | 1.36 | 6.78E−07 | 6.44E−06 |
| Trinucleotide repeat-containing gene 6B protein | TNRC6B | Q9UPQ9 | 0.98 | 0.59 | 1.37 | 6.97E−07 | 6.56E−06 |
| GSK3-beta interaction protein | GSKIP | Q9P0R6 | 0.97 | 0.44 | 1.5 | 3.58E−04 | 1.25E−03 |
| Histone-binding protein RBBP4 | RBBP4 | Q09028 | 0.97 | 0.49 | 1.45 | 7.59E−05 | 3.34E−04 |
| Cadherin-11: Extracellular domain | CDH11 | P55287 | 0.97 | 0.76 | 1.18 | 7.43E−20 | 6.17E−18 |
| Granulysin | GNLY | P22749 | 0.97 | 0.64 | 1.29 | 5.03E−09 | 8.27E−08 |
| Troponin T, cardiac muscle | TNNT2 | P45379 | 0.97 | 0.61 | 1.33 | 1.01E−07 | 1.20E−06 |
| CMP-N-acetylneuraminate-beta-galactosamide- | ST3GAL2 | Q16842 | 0.97 | 0.59 | 1.34 | 5.71E−07 | 5.60E−06 |
| alpha-2,3-sialyltransferase 2 | | | | | | | |
| E3 ubiquitin-protein ligase ZNRF3 | ZNRF3 | Q9ULT6 | 0.96 | 0.6 | 1.31 | 1.28E−07 | 1.50E−06 |
| Calcineurin B homologous protein 1 | CHP1 | Q99653 | 0.96 | 0.65 | 1.28 | 2.31E−09 | 4.09E−08 |
| 26S proteasome non-ATPase regulatory subunit 4 | PSMD4 | P55036 | 0.96 | 0.56 | 1.36 | 2.86E−06 | 2.17E−05 |
| Membrane-associated guanylate kinase, WW and | MAGI2 | Q86UL8 | 0.96 | 0.59 | 1.33 | 4.09E−07 | 4.27E−06 |
| PDZ domain-containing protein 2 | | | | | | | |
| YEATS domain-containing protein 4 | YEATS4 | O95619 | 0.96 | 0.33 | 1.58 | 2.79E−03 | 7.73E−03 |
| Transcription factor MafG | MAFG | O15525 | 0.96 | 0.64 | 1.29 | 7.37E−09 | 1.15E−07 |
| Interleukin-23 receptor | IL23R | Q5VWK5 | 0.96 | 0.5 | 1.42 | 4.89E−05 | 2.29E−04 |
| Tenascin | TNC | P24821 | 0.96 | 0.56 | 1.36 | 2.49E−06 | 1.93E−05 |
| Collagen alpha-1(IX) chain | COL9A1 | P20849 | 0.96 | 0.44 | 1.49 | 3.23E−04 | 1.14E−03 |
| Transmembrane glycoprotein NMB: Extracellular | GPNMB | Q14956 | 0.96 | 0.36 | 1.56 | 1.81E−03 | 5.24E−03 |
| domain | | | | | | | |
| Uroporphyrinogen-III synthase | UROS | P10746 | 0.95 | 0.58 | 1.32 | 3.85E−07 | 4.07E−06 |
| Peroxisomal trans-2-enoyl-CoA reductase | PECR | Q9BY49 | 0.95 | 0.47 | 1.42 | 9.41E−05 | 4.00E−04 |
| Lymphocyte antigen 86 | LY86 | O95711 | 0.95 | 0.38 | 1.52 | 1.16E−03 | 3.53E−03 |
| 40S ribosomal protein S7 | RPS7 | P62081 | 0.95 | 0.62 | 1.27 | 1.92E−08 | 2.68E−07 |
| Tyrosine-protein kinase Lck | LCK | P06239 | 0.95 | 0.62 | 1.28 | 1.25E−08 | 1.84E−07 |
| Rho GTPase-activating protein 36 | ARHGAP36 | Q6ZRI8 | 0.95 | 0.32 | 1.57 | 3.02E−03 | 8.30E−03 |
| Apelin | APLN | Q9ULZ1 | 0.95 | 0.3 | 1.59 | 3.97E−03 | 1.07E−02 |
| BMP and activin membrane-bound inhibitor | BAMBI | Q13145 | 0.95 | 0.45 | 1.44 | 1.76E−04 | 6.81E−04 |
| homolog: Extracellular domain | | | | | | | |
| Protein lin-7 homolog B | LIN7B | Q9HAP6 | 0.94 | 0.55 | 1.33 | 2.02E−06 | 1.62E−05 |
| Heterogeneous nuclear ribonucleoprotein D0 | HNRNPD | Q14103 | 0.94 | 0.64 | 1.24 | 1.36E−09 | 2.48E−08 |
| Clathrin light chain A | CLTA | P09496 | 0.94 | 0.6 | 1.28 | 4.26E−08 | 5.52E−07 |
| Transforming growth factor beta-1 | TGFB1 | P01137 | 0.94 | 0.51 | 1.37 | 1.95E−05 | 1.06E−04 |
| Lymphocyte antigen 6 complex locus protein G6c | LY6G6C | O95867 | 0.94 | 0.66 | 1.22 | 5.14E−11 | 1.22E−09 |
| Butyrophilin subfamily 2 member A1 | BTN2A1 | Q7KYR7 | 0.94 | 0.23 | 1.66 | 9.28E−03 | 2.26E−02 |
| Protein DGCR14 | DGCR14 | Q96DF8 | 0.93 | 0.48 | 1.37 | 4.53E−05 | 2.15E−04 |
| Complement component C1q receptor | CD93 | Q9NPY3 | 0.93 | 0.29 | 1.58 | 4.72E−03 | 1.24E−02 |
| StAR-related lipid transfer protein 5 | STARD5 | Q9NSY2 | 0.93 | 0.61 | 1.25 | 1.41E−08 | 2.06E−07 |
| ADP-ribosylation factor-like protein 5B | ARL5B | Q96KC2 | 0.93 | 0.42 | 1.45 | 4.08E−04 | 1.41E−03 |
| Protein phosphatase 1 regulatory subunit 1A | PPP1R1A | Q13522 | 0.93 | 0.37 | 1.48 | 1.12E−03 | 3.44E−03 |
| Complement factor D | CFD | P00746 | 0.93 | 0.34 | 1.52 | 1.89E−03 | 5.43E−03 |
| Pleiotrophin | PTN | P21246 | 0.93 | 0.56 | 1.3 | 6.81E−07 | 6.45E−06 |
| Uncharacterized protein C11orf94 | C11orf94 | C9JXX5 | 0.93 | 0.44 | 1.42 | 1.80E−04 | 6.93E−04 |
| Ubiquitin-conjugating enzyme E2 E1 | UBE2E1 | P51965 | 0.92 | 0.68 | 1.17 | 1.02E−13 | 3.84E−12 |
| Regenerating islet-derived protein 3-alpha | REG3A | Q06141 | 0.92 | 0.66 | 1.18 | 4.66E−12 | 1.33E−10 |
| Astrocytic phosphoprotein PEA-15 | PEA15 | Q15121 | 0.92 | 0.49 | 1.35 | 3.03E−05 | 1.53E−04 |
| GDNF family receptor alpha-1 | GFRA1 | P56159 | 0.92 | 0.35 | 1.48 | 1.40E−03 | 4.15E−03 |
| Tumor necrosis factor | TNF | P01375 | 0.92 | 0.15 | 1.69 | 1.91E−02 | 4.27E−02 |
| Urotensin-2B | UTS2B | Q765I0 | 0.92 | 0.44 | 1.39 | 1.45E−04 | 5.75E−04 |
| Enhancer of rudimentary homolog | ERH | P84090 | 0.91 | 0.61 | 1.2 | 1.59E−09 | 2.86E−08 |
| DNA-3-methyladenine glycosylase | MPG | P29372 | 0.91 | 0.52 | 1.3 | 5.71E−06 | 3.91E−05 |
| TOX high mobility group box family member 3 | TOX3 | O15405 | 0.91 | 0.54 | 1.28 | 1.48E−06 | 1.25E−05 |
| 14 kDa phosphohistidine phosphatase | PHPT1 | Q9NRX4 | 0.91 | 0.54 | 1.28 | 1.20E−06 | 1.05E−05 |
| Glutaredoxin-1 | GLRX | P35754 | 0.91 | 0.39 | 1.43 | 6.18E−04 | 2.02E−03 |
| Natural cytotoxicity triggering receptor 2 | NCR2 | O95944 | 0.91 | 0.37 | 1.45 | 1.01E−03 | 3.14E−03 |
| Fibroblast growth factor 7 | FGF7 | P21781 | 0.91 | 0.57 | 1.26 | 2.02E−07 | 2.29E−06 |
| Inter-alpha-trypsin inhibitor heavy chain H4 | ITIH4 | Q14624 | 0.91 | 0.17 | 1.65 | 1.65E−02 | 3.78E−02 |
| Putative chondrosarcoma-associated gene 1 | CSAG1 | Q6PB30 | 0.91 | 0.4 | 1.43 | 5.23E−04 | 1.76E−03 |
| protein | | | | | | | |
| Myeloid-derived growth factor | MYDGF | Q969H8 | 0.91 | 0.39 | 1.42 | 5.75E−04 | 1.90E−03 |
| Selenoprotein W | SEPW1 | P63302 | 0.9 | 0.6 | 1.21 | 5.46E−09 | 8.86E−08 |
| Cyclin-dependent kinase 1: G2/mitotic-specific | CDC2 CCNB1 | P14635\|P06493 | 0.9 | 0.3 | 1.5 | 3.40E−03 | 9.25E−03 |
| cyclin-B1 complex | | | | | | | |
| Acidic leucine-rich nuclear phosphoprotein 32 | ANP32B | Q92688 | 0.9 | 0.35 | 1.45 | 1.44E−03 | 4.24E−03 |
| family member B | | | | | | | |
| Scavenger receptor class F member | SCARF2 | Q96GP6 | 0.9 | 0.13 | 1.67 | 2.21E−02 | 4.86E−02 |
| 2: Extracellular domain | | | | | | | |
| tRNA pseudouridine synthase A, mitochondrial | PUS1 | Q9Y606 | 0.89 | 0.48 | 1.31 | 2.36E−05 | 1.24E−04 |
| Selenoprotein M | SELM | Q8WWX9 | 0.89 | 0.48 | 1.31 | 2.59E−05 | 1.34E−04 |
| Serum amyloid A-4 protein | SAA4 | P35542 | 0.89 | 0.47 | 1.3 | 2.96E−05 | 1.51E−04 |
| RNA-binding protein 39 | RBM39 | Q14498 | 0.89 | 0.53 | 1.26 | 1.71E−06 | 1.41E−05 |
| C-reactive protein | CRP | P02741 | 0.89 | 0.72 | 1.05 | 2.45E−26 | 5.55E−24 |
| Microfibril-associated glycoprotein 4 | MFAP4 | P55083 | 0.89 | 0.52 | 1.26 | 2.43E−06 | 1.90E−05 |
| Protein S100-A12 | S100A12 | P80511 | 0.89 | 0.57 | 1.21 | 5.08E−08 | 6.47E−07 |
| PolyUbiquitin K48-linked | UBB | P0CG47 | 0.89 | 0.48 | 1.3 | 1.90E−05 | 1.04E−04 |
| Leukocyte immunoglobulin-like receptor | LILRA5 | A6NI73 | 0.89 | 0.47 | 1.31 | 3.34E−05 | 1.66E−04 |
| subfamily A member 5 | | | | | | | |
| Cysteine and glycine-rich protein 3 | CSRP3 | P50461 | 0.89 | 0.51 | 1.27 | 4.21E−06 | 3.02E−05 |
| Peptidyl-prolyl cis-trans isomerase FKBP7 | FKBP7 | Q9Y680 | 0.89 | 0.62 | 1.17 | 1.80E−10 | 3.85E−09 |
| Syntaxin-12 | STX12 | Q86Y82 | 0.88 | 0.58 | 1.18 | 1.10E−08 | 1.65E−07 |
| RNA binding protein fox-1 homolog 2 | RBFOX2 | O43251 | 0.88 | 0.35 | 1.41 | 1.16E−03 | 3.52E−03 |
| Ras-related protein Rab-14 | RAB14 | P61106 | 0.88 | 0.53 | 1.23 | 9.39E−07 | 8.40E−06 |
| Guanylate-binding protein 2 | GBP2 | P32456 | 0.88 | 0.4 | 1.36 | 3.05E−04 | 1.09E−03 |
| Neutrophil gelatinase-associated lipocalin | LCN2 | P80188 | 0.88 | 0.51 | 1.25 | 2.78E−06 | 2.12E−05 |
| Serine/threonine-protein kinase TBK1 | TBK1 | Q9UHD2 | 0.88 | 0.44 | 1.32 | 8.30E−05 | 3.59E−04 |
| Granzyme A | GZMA | P12544 | 0.88 | 0.44 | 1.32 | 8.25E−05 | 3.58E−04 |
| alpha-Fetoprotein | AFP | P02771 | 0.88 | 0.42 | 1.34 | 1.71E−04 | 6.67E−04 |
| Glutaredoxin-2, mitochondrial | GLRX2 | Q9NS18 | 0.87 | 0.46 | 1.27 | 3.05E−05 | 1.54E−04 |
| E3 ubiquitin-protein ligase NRDP1 | RNF41 | Q9H4P4 | 0.87 | 0.4 | 1.35 | 2.90E−04 | 1.05E−03 |
| Peptidyl-prolyl cis-trans isomerase FKBP4 | FKBP4 | Q02790 | 0.87 | 0.56 | 1.17 | 3.50E−08 | 4.61E−07 |
| Tyrosine-protein kinase Yes | YES1 | P07947 | 0.87 | 0.36 | 1.38 | 7.81E−04 | 2.48E−03 |
| Low-density lipoprotein receptor-related protein | LRP11 | Q86VZ4 | 0.87 | 0.4 | 1.34 | 3.01E−04 | 1.08E−03 |
| 11 | | | | | | | |
| ADP-ribosylation factor-like protein 8B | ARL8B | Q9NVJ2 | 0.87 | 0.32 | 1.43 | 1.98E−03 | 5.65E−03 |
| Heterogeneous nuclear ribonucleoprotein A/B | HNRNPAB | Q99729 | 0.87 | 0.51 | 1.22 | 2.40E−06 | 1.88E−05 |
| DNA-(apurinic or apyrimidinic site) lyase | APEX1 | P27695 | 0.87 | 0.59 | 1.15 | 9.32E−10 | 1.76E−08 |
| Cullin-4B | CUL4B | Q13620 | 0.86 | 0.52 | 1.2 | 7.98E−07 | 7.33E−06 |
| Poly(ADP-ribose) glycohydrolase ARH3 | ADPRHL2 | Q9NX46 | 0.86 | 0.6 | 1.13 | 2.77E−10 | 5.72E−09 |
| Tumor necrosis factor receptor superfamily | EDA2R | Q9HAV5 | 0.86 | 0.43 | 1.3 | 1.02E−04 | 4.31E−04 |
| member 27 | | | | | | | |
| Stathmin-2 | STMN2 | Q93045 | 0.85 | 0.58 | 1.12 | 5.56E−10 | 1.11E−08 |
| Histone acetyltransferase KAT2B | KAT2B | Q92831 | 0.85 | 0.33 | 1.36 | 1.25E−03 | 3.78E−03 |
| Rho-related GTP-binding protein RhoG | RHOG | P84095 | 0.85 | 0.3 | 1.4 | 2.48E−03 | 6.97E−03 |
| Urea transporter 2 | SLC14A2 | Q15849 | 0.85 | 0.36 | 1.35 | 6.85E−04 | 2.21E−03 |
| Serum amyloid A-1 protein | SAA1 | PODJI8 | 0.85 | 0.69 | 1 | 2.89E−26 | 6.26E−24 |
| Aldose reductase | AKR1B1 | P15121 | 0.85 | 0.53 | 1.17 | 2.13E−07 | 2.40E−06 |
| Calcium/calmodulin-dependent protein kinase | CAMKK1 | Q8N5S9 | 0.85 | 0.24 | 1.45 | 6.09E−03 | 1.56E−02 |
| kinase 1 | | | | | | | |
| Protein FAM49B | FAM49B | Q9NUQ9 | 0.85 | 0.59 | 1.11 | 2.61E−10 | 5.42E−09 |
| Membrane frizzled-related protein | MFRP | Q9BY79 | 0.85 | 0.48 | 1.22 | 7.76E−06 | 5.03E−05 |
| Scavenger receptor class F member 1 | SCARF1 | Q14162 | 0.85 | 0.52 | 1.18 | 5.13E−07 | 5.14E−06 |
| Nesprin-2 | SYNE2 | Q8WXH0 | 0.85 | 0.52 | 1.18 | 3.67E−07 | 3.92E−06 |
| Serine/threonine-protein kinase BRSK2 | BRSK2 | Q8IWQ3 | 0.85 | 0.36 | 1.34 | 6.52E−04 | 2.12E−03 |
| Procollagen-lysine, 2-oxoglutarate 5-dioxygenase | PLOD3 | O60568 | 0.84 | 0.4 | 1.28 | 1.93E−04 | 7.40E−04 |
| 3 | | | | | | | |
| C-type lectin domain family 4 member G | CLEC4G | Q6UXB4 | 0.84 | 0.33 | 1.36 | 1.36E−03 | 4.05E−03 |
| Phosphatidylcholine-sterol acyltransferase | LCAT | P04180 | 0.84 | 0.43 | 1.25 | 4.94E−05 | 2.31E−04 |
| Guanine nucleotide exchange factor MSS4 | RABIF | P47224 | 0.84 | 0.4 | 1.28 | 1.79E−04 | 6.91E−04 |
| Insulin-degrading enzyme | IDE | P14735 | 0.84 | 0.39 | 1.28 | 2.15E−04 | 8.13E−04 |
| Transforming growth factor beta-3 | TGFB3 | P10600 | 0.84 | 0.31 | 1.38 | 1.98E−03 | 5.65E−03 |
| Caspase recruitment domain-containing protein | C9orf89 | Q96LW7 | 0.84 | 0.13 | 1.55 | 2.07E−02 | 4.60E−02 |
| 19 | | | | | | | |
| Matrix-remodeling-associated protein 7 | MXRA7 | P84157 | 0.84 | 0.36 | 1.32 | 5.43E−04 | 1.81E−03 |
| Spliceosome RNA helicase DDX39B | DDX39B | Q13838 | 0.84 | 0.5 | 1.18 | 1.26E−06 | 1.09E−05 |
| Retinol dehydrogenase 16 | RDH16 | O75452 | 0.83 | 0.35 | 1.3 | 6.71E−04 | 2.17E−03 |
| Envoplakin | EVPL | Q92817 | 0.83 | 0.35 | 1.31 | 7.53E−04 | 2.40E−03 |
| Structural maintenance of chromosomes protein 3 | SMC3 | Q9UQE7 | 0.83 | 0.53 | 1.13 | 4.75E−08 | 6.10E−07 |
| DCN1-like protein 1 | DCUN1D1 | Q96GG9 | 0.83 | 0.21 | 1.45 | 9.19E−03 | 2.25E−02 |
| Hsc70-interacting protein | ST13 | P50502 | 0.83 | 0.42 | 1.24 | 8.36E−05 | 3.62E−04 |
| Tumor necrosis factor receptor superfamily | TNFRSF4 | P43489 | 0.83 | 0.2 | 1.46 | 9.71E−03 | 2.35E−02 |
| member 4 | | | | | | | |
| Interleukin-6 | IL6 | P05231 | 0.83 | 0.58 | 1.08 | 7.12E−11 | 1.65E−09 |
| Transmembrane protein PVRIG | PVRIG | Q6DKI7 | 0.83 | 0.29 | 1.36 | 2.57E−03 | 7.19E−03 |
| Palmitoyl-protein thioesterase 1 | PPT1 | P50897 | 0.83 | 0.52 | 1.14 | 1.89E−07 | 2.16E−06 |
| Prokineticin-2 | PROK2 | Q9HC23 | 0.82 | 0.58 | 1.05 | 8.98E−12 | 2.44E−10 |
| Splicing factor 1 | SF1 | Q15637 | 0.82 | 0.26 | 1.39 | 4.26E−03 | 1.13E−02 |
| Acyl-CoA-binding protein | DBI | P07108 | 0.82 | 0.58 | 1.05 | 1.24E−11 | 3.30E−10 |
| Killer cell immunoglobulin-like receptor 3DL1 | KIR3DL1 | P43629 | 0.82 | 0.24 | 1.39 | 5.47E−03 | 1.42E−02 |
| NKG2D ligand 3 | ULBP3 | Q9BZM4 | 0.82 | 0.49 | 1.15 | 1.19E−06 | 1.04E−05 |
| Proteasome subunit alpha type-6 | PSMA6 | P60900 | 0.82 | 0.48 | 1.16 | 2.48E−06 | 1.93E−05 |
| Fibroblast growth factor 4 | FGF4 | P08620 | 0.82 | 0.4 | 1.23 | 1.11E−04 | 4.62E−04 |
| Mitochondrial import inner membrane | DNAJC19 | Q96DA6 | 0.82 | 0.45 | 1.19 | 1.57E−05 | 8.91E−05 |
| translocase subunit TIM14 | | | | | | | |
| Glucokinase regulatory protein | GCKR | Q14397 | 0.82 | 0.23 | 1.41 | 6.57E−03 | 1.68E−02 |
| Tenascin | TNC | P24821 | 0.82 | 0.46 | 1.17 | 6.91E−06 | 4.58E−05 |
| Acylphosphatase-2 | ACYP2 | P14621 | 0.81 | 0.55 | 1.07 | 8.80E−10 | 1.69E−08 |
| Calponin-1 | CNN1 | P51911 | 0.81 | 0.38 | 1.24 | 2.40E−04 | 8.93E−04 |
| CD40 ligand | CD40LG | P29965 | 0.81 | 0.5 | 1.13 | 3.44E−07 | 3.71E−06 |
| Methyl-CpG-binding domain protein 4 | MBD4 | O95243 | 0.81 | 0.39 | 1.23 | 1.40E−04 | 5.63E−04 |
| Leukocyte immunoglobulin-like receptor | LILRB2 | Q8N423 | 0.81 | 0.21 | 1.41 | 8.25E−03 | 2.04E−02 |
| subfamily B member 2 | | | | | | | |
| Cartilage intermediate layer protein 1 | CILP | O75339 | 0.81 | 0.57 | 1.05 | 7.12E−11 | 1.65E−09 |
| Protocadherin-8 | PCDH8 | O95206 | 0.81 | 0.51 | 1.11 | 1.36E−07 | 1.59E−06 |
| Serpin A11 | SERPINA11 | Q86U17 | 0.81 | 0.23 | 1.39 | 6.00E−03 | 1.54E−02 |
| Inositol 1,4,5-trisphosphate receptor-interacting | ITPRIPL1 | Q6GPH6 | 0.81 | 0.49 | 1.13 | 7.07E−07 | 6.59E−06 |
| protein-like 1 | | | | | | | |
| Low-density lipoprotein receptor-related protein | LRP1B | Q9NZR2 | 0.8 | 0.4 | 1.2 | 1.02E−04 | 4.29E−04 |
| 1B | | | | | | | |
| Double-stranded RNA-binding protein Staufen | STAU1 | O95793 | 0.8 | 0.45 | 1.16 | 1.03E−05 | 6.30E−05 |
| homolog 1 | | | | | | | |
| Protein phosphatase 1A | PPM1A | P35813 | 0.8 | 0.43 | 1.17 | 2.10E−05 | 1.12E−04 |
| Retinoid-binding protein 7 | RBP7 | Q96R05 | 0.8 | 0.21 | 1.38 | 7.88E−03 | 1.96E−02 |
| Gastric inhibitory polypeptide | GIP | P09681 | 0.8 | 0.51 | 1.09 | 5.42E−08 | 6.81E−07 |
| Mimecan | OGN | P20774 | 0.8 | 0.4 | 1.21 | 1.09E−04 | 4.54E−04 |
| A disintegrin and metalloproteinase with | ADAMTS1 | Q9UHI8 | 0.8 | 0.37 | 1.24 | 3.17E−04 | 1.13E−03 |
| thrombospondin motifs 1 | | | | | | | |
| Tissue Factor | F3 | P13726 | 0.8 | 0.34 | 1.25 | 6.30E−04 | 2.05E−03 |
| Semaphorin-6B | SEMA6B | Q9H3T3 | 0.8 | 0.27 | 1.33 | 3.18E−03 | 8.72E−03 |
| Fas apoptotic inhibitory molecule 3 | FAIM3 | O60667 | 0.8 | 0.48 | 1.11 | 6.11E−07 | 5.96E−06 |
| Extracellular sulfatase Sulf-2 | SULF2 | Q8IWU5 | 0.8 | 0.32 | 1.28 | 9.99E−04 | 3.10E−03 |
| DNA repair protein XRCC4 | XRCC4 | Q13426 | 0.8 | 0.49 | 1.12 | 6.01E−07 | 5.86E−06 |
| Membrane-associated progesterone receptor | PGRMC2 | O15173 | 0.79 | 0.45 | 1.13 | 6.15E−06 | 4.14E−05 |
| component 2 | | | | | | | |
| Dual specificity protein phosphatase 6 | DUSP6 | Q16828 | 0.79 | 0.23 | 1.36 | 5.84E−03 | 1.50E−02 |
| AMSH-like protease | STAMBPL1 | Q96FJ0 | 0.79 | 0.27 | 1.31 | 2.97E−03 | 8.20E−03 |
| Serine/threonine-protein kinase ULK3 | ULK3 | Q6PHR2 | 0.79 | 0.16 | 1.43 | 1.35E−02 | 3.17E−02 |
| 60S ribosomal protein L30 | RPL30 | P62888 | 0.79 | 0.54 | 1.03 | 5.71E−10 | 1.14E−08 |
| Tudor-interacting repair regulator protein | NUDT16L1 | Q9BRJ7 | 0.79 | 0.53 | 1.04 | 1.06E−09 | 1.98E−08 |
| Dynein light chain Tetex-type 3 | DYNLT3 | P51808 | 0.79 | 0.25 | 1.32 | 3.86E−03 | 1.04E−02 |
| DNA-binding protein inhibitor ID-2 | ID2 | Q02363 | 0.79 | 0.34 | 1.24 | 5.07E−04 | 1.71E−03 |
| DnaJ homolog subfamily B member 4 | DNAJB4 | Q9UDY4 | 0.79 | 0.55 | 1.03 | 7.46E−11 | 1.71E−09 |
| Platelet-activating factor acetylhydrolase IB | PAFAH1B2 | P68402 | 0.79 | 0.53 | 1.05 | 2.96E−09 | 5.08E−08 |
| subunit beta | | | | | | | |
| Kunitz-type protease inhibitor 2 | SPINT2 | O43291 | 0.79 | 0.47 | 1.11 | 1.32E−06 | 1.12E−05 |
| Thymic stromal lymphopoietin | TSLP | Q969D9 | 0.79 | 0.3 | 1.28 | 1.46E−03 | 4.29E−03 |
| Low affinity immunoglobulin epsilon Fc receptor | FCER2 | P06734 | 0.79 | 0.46 | 1.12 | 2.95E−06 | 2.23E−05 |
| Apoptosis regulator Bcl-2 | BCL2 | P10415 | 0.79 | 0.46 | 1.13 | 2.42E−06 | 1.90E−05 |
| Legumain | LGMN | Q99538 | 0.79 | 0.18 | 1.41 | 1.16E−02 | 2.77E−02 |
| SLAM family member 6 | SLAMF6 | Q96DU3 | 0.79 | 0.3 | 1.28 | 1.67E−03 | 4.85E−03 |
| SLAM family member 7 | SLAMF7 | Q9NQ25 | 0.79 | 0.54 | 1.04 | 1.17E−09 | 2.14E−08 |
| Protein phosphatase 1L | PPM1L | Q5SGD2 | 0.79 | 0.48 | 1.09 | 4.46E−07 | 4.61E−06 |
| Synaptotagmin-17 | SYT17 | Q9BSW7 | 0.79 | 0.31 | 1.27 | 1.17E−03 | 3.56E−03 |
| Sialate O-acetylesterase | SIAE | Q9HAT2 | 0.79 | 0.48 | 1.1 | 5.22E−07 | 5.19E−06 |
| Protocadherin beta-10 | PCDHB10 | Q9UN67 | 0.79 | 0.31 | 1.26 | 1.13E−03 | 3.46E−03 |
| Heat shock factor protein 1 | HSF1 | Q00613 | 0.78 | 0.19 | 1.36 | 9.24E−03 | 2.25E−02 |
| Discoidin domain-containing receptor 2 | DDR2 | Q16832 | 0.78 | 0.34 | 1.21 | 4.47E−04 | 1.53E−03 |
| Tumor necrosis factor receptor superfamily | TNFRSF13B | O14836 | 0.78 | 0.11 | 1.44 | 2.21E−02 | 4.86E−02 |
| member 13B | | | | | | | |
| Proteasome subunit alpha type-1 | PSMA1 | P25786 | 0.78 | 0.19 | 1.37 | 9.34E−03 | 2.27E−02 |
| Annexin A2 | ANXA2 | P07355 | 0.78 | 0.41 | 1.15 | 3.93E−05 | 1.91E−04 |
| Intercellular adhesion molecule 5 | ICAM5 | Q9UMF0 | 0.78 | 0.41 | 1.15 | 3.57E−05 | 1.75E−04 |
| Mitochondrial glutamate carrier 2 | SLC25A18 | Q9H1K4 | 0.78 | 0.24 | 1.32 | 4.51E−03 | 1.19E−02 |
| LEM domain-containing protein 1 | LEMD1 | Q68G75 | 0.78 | 0.48 | 1.08 | 2.74E−07 | 3.01E−06 |
| CUB and sushi domain-containing protein 1 | CSMD1 | Q96PZ7 | 0.78 | 0.36 | 1.2 | 2.97E−04 | 1.07E−03 |
| MAP/microtubule affinity-regulating kinase 3 | MARK3 | P27448 | 0.78 | 0.36 | 1.2 | 2.42E−04 | 8.98E−04 |
| Steroidogenic acute regulatory protein, | STAR | P49675 | 0.77 | 0.29 | 1.26 | 1.67E−03 | 4.86E−03 |
| mitochondrial | | | | | | | |
| Cyclic AMP-responsive element-binding protein | CREB3L4 | Q8TEY5 | 0.77 | 0.21 | 1.34 | 7.52E−03 | 1.88E−02 |
| 3-like protein 4 | | | | | | | |
| Amyloid beta A4 precursor protein-binding | APBB2 | Q92870 | 0.77 | 0.25 | 1.29 | 3.89E−03 | 1.05E−02 |
| family B member 2: Phosphotyrosine Interaction | | | | | | | |
| Domain 1 | | | | | | | |
| Left-right determination factor 2 | LEFTY2 | O00292 | 0.77 | 0.35 | 1.2 | 3.82E−04 | 1.33E−03 |
| Transcription elongation factor A protein 1 | TCEA1 | P23193 | 0.77 | 0.47 | 1.08 | 5.54E−07 | 5.45E−06 |
| N-acetylneuraminate lyase | NPL | Q9BXD5 | 0.77 | 0.33 | 1.22 | 6.63E−04 | 2.15E−03 |
| Decorin | DCN | P07585 | 0.77 | 0.32 | 1.22 | 7.86E−04 | 2.49E−03 |
| C-C motif chemokine 17 | CCL17 | Q92583 | 0.77 | 0.51 | 1.03 | 6.64E−09 | 1.05E−07 |
| Interleukin-20 | IL20 | Q9NYY1 | 0.77 | 0.43 | 1.1 | 8.30E−06 | 5.30E−05 |
| Tenascin | TNC | P24821 | 0.77 | 0.43 | 1.11 | 1.10E−05 | 6.62E−05 |
| Glycolipid transfer protein domain-containing | GLTPD2 | A6NH11 | 0.77 | 0.23 | 1.31 | 4.91E−03 | 1.29E−02 |
| protein 2 | | | | | | | |
| Colipase-like protein 1 | CLPSL1 | A2RUU4 | 0.77 | 0.35 | 1.2 | 3.81E−04 | 1.33E−03 |
| Polyadenylate-binding protein 3 | PABPC3 | Q9H361 | 0.76 | 0.46 | 1.06 | 9.57E−07 | 8.54E−06 |
| Nuclear RNA export factor 1 | NXF1 | Q9UBU9 | 0.76 | 0.3 | 1.22 | 1.13E−03 | 3.46E−03 |
| cAMP-dependent protein kinase type I-beta | PRKAR1B | P31321 | 0.76 | 0.48 | 1.04 | 1.10E−07 | 1.30E−06 |
| regulatory subunit | | | | | | | |
| Protein canopy homolog 3 | CNPY3 | Q9BT09 | 0.76 | 0.51 | 1.01 | 5.27E−09 | 8.63E−08 |
| Adhesion G-protein coupled receptor G1 | GPR56 | Q9Y653 | 0.76 | 0.37 | 1.14 | 1.17E−04 | 4.81E−04 |
| Angiotensin-converting enzyme 2 | ACE2 | Q9BYF1 | 0.76 | 0.33 | 1.19 | 5.53E−04 | 1.84E−03 |
| Midkine | MDK | P21741 | 0.76 | 0.38 | 1.14 | 8.73E−05 | 3.76E−04 |
| AT-rich interactive domain-containing protein 3A | ARID3A | Q99856 | 0.76 | 0.46 | 1.06 | 4.87E−07 | 4.95E−06 |
| NudC domain-containing protein 3 | NUDCD3 | Q8IVD9 | 0.76 | 0.38 | 1.14 | 1.02E−04 | 4.29E−04 |
| Hepatoma-derived growth factor-related protein 2 | HDGFRP2 | Q7Z4V5 | 0.76 | 0.18 | 1.34 | 1.06E−02 | 2.54E−02 |
| Inhibin beta C chain | INHBC | P55103 | 0.76 | 0.22 | 1.29 | 5.63E−03 | 1.46E−02 |
| Disks large homolog 3 | DLG3 | Q92796 | 0.76 | 0.32 | 1.21 | 8.26E−04 | 2.61E−03 |
| Protein polybromo-1 | PBRM1 | Q86U86 | 0.75 | 0.34 | 1.16 | 2.93E−04 | 1.06E−03 |
| RNA-binding protein 3 | RBM3 | P98179 | 0.75 | 0.51 | 0.98 | 7.20E−10 | 1.41E−08 |
| BMP-binding endothelial regulator protein | BMPER | Q8N8U9 | 0.75 | 0.18 | 1.32 | 9.76E−03 | 2.36E−02 |
| Beta-crystallin B1 | CRYBB1 | P53674 | 0.75 | 0.46 | 1.04 | 4.99E−07 | 5.02E−06 |
| Caspase recruitment domain-containing protein | CARD17 | Q5XLA6 | 0.75 | 0.17 | 1.33 | 1.16E−02 | 2.76E−02 |
| 17 | | | | | | | |
| Alpha-ketoglutarate-dependent dioxygenase alkB | ALKBH3 | Q96Q83 | 0.75 | 0.49 | 1.01 | 1.88E−08 | 2.64E−07 |
| homolog 3 | | | | | | | |
| 40S ribosomal protein S5 | RPS5 | P46782 | 0.75 | 0.46 | 1.04 | 3.67E−07 | 3.92E−06 |
| Protein tyrosine phosphatase type IVA 3 | PTP4A3 | O75365 | 0.75 | 0.35 | 1.15 | 2.21E−04 | 8.33E−04 |
| Fibroblast growth factor 8 | FGF8 | P55075 | 0.75 | 0.17 | 1.33 | 1.12E−02 | 2.67E−02 |
| Angiopoietin-4 | ANGPT4 | Q9Y264 | 0.75 | 0.39 | 1.12 | 4.13E−05 | 1.99E−04 |
| Diablo homolog, mitochondrial | DIABLO | Q9NR28 | 0.75 | 0.25 | 1.26 | 3.33E−03 | 9.09E−03 |
| Brain-specific serine protease 4 | PRSS22 | Q9GZN4 | 0.75 | 0.24 | 1.26 | 4.20E−03 | 1.12E−02 |
| Protein kinase C gamma type | PRKCG | P05129 | 0.75 | 0.18 | 1.31 | 9.38E−03 | 2.28E−02 |
| Corneodesmosin | CDSN | Q15517 | 0.75 | 0.42 | 1.09 | 1.03E−05 | 6.30E−05 |
| Leptin | LEP | P41159 | 0.75 | 0.6 | 0.9 | 3.49E−22 | 4.04E−20 |
| Activator of 90 kDa heat shock protein ATPase | AHSA1 | O95433 | 0.74 | 0.4 | 1.08 | 1.98E−05 | 1.07E−04 |
| homolog 1 | | | | | | | |
| U1 small nuclear ribonucleoprotein A | SNRPA | P09012 | 0.74 | 0.45 | 1.04 | 7.18E−07 | 6.68E−06 |
| CREB-binding protein | CREBBP | Q92793 | 0.74 | 0.28 | 1.2 | 1.65E−03 | 4.80E−03 |
| Cyclin-dependent kinase inhibitor 1 | CDKN1A | P38936 | 0.74 | 0.41 | 1.06 | 8.88E−06 | 5.58E−05 |
| Complement C3b, inactivated | C3 | P01024 | 0.74 | 0.42 | 1.07 | 8.87E−06 | 5.58E−05 |
| Vasoactive Intestinal Peptide | VIP | P01282 | 0.74 | 0.41 | 1.06 | 9.92E−06 | 6.12E−05 |
| C-type lectin domain family 7 member A | CLEC7A | Q9BXN2 | 0.74 | 0.36 | 1.13 | 1.69E−04 | 6.60E−04 |
| Fibroblast growth factor 8 isoform A | FGF8 | P55075 | 0.74 | 0.43 | 1.04 | 2.76E−06 | 2.12E−05 |
| 14-3-3 protein sigma | SFN | P31947 | 0.74 | 0.31 | 1.17 | 7.65E−04 | 2.44E−03 |
| 40S ribosomal protein S3a | RPS3A | P61247 | 0.74 | 0.37 | 1.12 | 1.03E−04 | 4.35E−04 |
| Secretoglobin family 3A member 1 | SCGB3A1 | Q96QR1 | 0.74 | 0.5 | 0.99 | 4.54E−09 | 7.53E−08 |
| Carboxypeptidase Z | CPZ | Q66K79 | 0.74 | 0.3 | 1.18 | 1.02E−03 | 3.16E−03 |
| SH3 domain-binding glutamic acid-rich-like | SH3BGRL | O75368 | 0.73 | 0.45 | 1.01 | 2.99E−07 | 3.26E−06 |
| protein | | | | | | | |
| Nuclear envelope phosphatase-regulatory subunit | CNEP1R1 | Q8N9A8 | 0.73 | 0.19 | 1.28 | 8.25E−03 | 2.04E−02 |
| 1 | | | | | | | |
| Probable RNA-binding protein EIF1AD | EIF1AD | Q8N9N8 | 0.73 | 0.49 | 0.98 | 6.37E−09 | 1.01E−07 |
| Actin-related protein 2/3 complex subunit 3 | ARPC3 | O15145 | 0.73 | 0.32 | 1.13 | 4.46E−04 | 1.52E−03 |
| Alpha-galactosidase A | GLA | P06280 | 0.73 | 0.32 | 1.14 | 5.24E−04 | 1.76E−03 |
| Methionine-R-sulfoxide reductase B2, | MSRB2 | Q9Y3D2 | 0.73 | 0.24 | 1.22 | 3.58E−03 | 9.69E−03 |
| mitochondrial | | | | | | | |
| Protein NDRG3 | NDRG3 | Q9UGV2 | 0.73 | 0.37 | 1.1 | 6.60E−05 | 2.97E−04 |
| 40S ribosomal protein S19 | RPS19 | P39019 | 0.73 | 0.44 | 1.02 | 1.21E−06 | 1.05E−05 |
| A disintegrin and metalloproteinase with | ADAMTS5 | Q9UNA0 | 0.73 | 0.13 | 1.34 | 1.71E−02 | 3.88E−02 |
| thrombospondin motifs 5 | | | | | | | |
| Proto-oncogene tyrosine-protein kinase receptor | RET | P07949 | 0.73 | 0.36 | 1.1 | 9.94E−05 | 4.20E−04 |
| Ret | | | | | | | |
| Angiopoietin-related protein 4 | ANGPTL4 | Q9BY76 | 0.73 | 0.19 | 1.28 | 8.38E−03 | 2.07E−02 |
| Dual specificity mitogen-activated protein kinase | MAP2K3 | P46734 | 0.73 | 0.36 | 1.09 | 9.50E−05 | 4.03E−04 |
| kinase 3 | | | | | | | |
| Rho GDP-dissociation inhibitor 1 | ARHGDIA | P52565 | 0.73 | 0.17 | 1.29 | 1.06E−02 | 2.54E−02 |
| Trafficking protein particle complex subunit 4 | TRAPPC4 | Q9Y296 | 0.73 | 0.24 | 1.22 | 3.44E−03 | 9.34E−03 |
| Immunoglobulin superfamily member 3 | IGSF3 | O75054 | 0.73 | 0.28 | 1.17 | 1.35E−03 | 4.04E−03 |
| ETS domain-containing protein Elk-1 | ELK1 | P19419 | 0.72 | 0.18 | 1.26 | 8.69E−03 | 2.14E−02 |
| UV excision repair protein RAD23 homolog A | RAD23A | P54725 | 0.72 | 0.47 | 0.97 | 1.72E−08 | 2.45E−07 |
| Probable ATP-dependent RNA helicase DDX58 | DDX58 | O95786 | 0.72 | 0.24 | 1.2 | 3.31E−03 | 9.04E−03 |
| Double-stranded RNA-binding protein Staufen | STAU2 | Q9NUL3 | 0.72 | 0.18 | 1.26 | 8.45E−03 | 2.08E−02 |
| homolog 2 | | | | | | | |
| Interleukin-15 receptor subunit alpha | IL15RA | Q13261 | 0.72 | 0.37 | 1.07 | 4.97E−05 | 2.32E−04 |
| Ubiquitin-like protein ISG15 | ISG15 | P05161 | 0.72 | 0.48 | 0.96 | 4.49E−09 | 7.47E−08 |
| BolA-like protein 1 | BOLA1 | Q9Y3E2 | 0.72 | 0.39 | 1.06 | 2.37E−05 | 1.25E−04 |
| Eukaryotic initiation factor 4A-II | EIF4A2 | Q14240 | 0.72 | 0.43 | 1 | 1.02E−06 | 9.02E−06 |
| Erythropoietin receptor | EPOR | P19235 | 0.72 | 0.21 | 1.22 | 5.23E−03 | 1.37E−02 |
| Inhibitor of growth protein 1 | ING1 | Q9UK53 | 0.72 | 0.44 | 1 | 4.50E−07 | 4.64E−06 |
| Insulin | INS | P01308 | 0.72 | 0.44 | 1.01 | 7.60E−07 | 7.04E−06 |
| Thrombopoietin | THPO | P40225 | 0.72 | 0.41 | 1.04 | 6.77E−06 | 4.50E−05 |
| Leukocyte surface antigen CD47 | CD47 | Q08722 | 0.72 | 0.38 | 1.06 | 2.97E−05 | 1.51E−04 |
| Rho GTPase-activating protein 1: Cellular | ARHGAP1 | Q07960 | 0.72 | 0.4 | 1.05 | 1.02E−05 | 6.28E−05 |
| retinaldehyde-TRIO domain | | | | | | | |
| Voltage-gated potassium channel subunit beta-2 | KCNAB2 | Q13303 | 0.71 | 0.27 | 1.14 | 1.40E−03 | 4.16E−03 |
| Tyrosyl-DNA phosphodiesterase 1 | TDP1 | Q9NUW8 | 0.71 | 0.24 | 1.18 | 3.33E−03 | 9.10E−03 |
| E3 SUMO-protein ligase PIAS4 | PIAS4 | Q8N2W9 | 0.71 | 0.11 | 1.3 | 2.09E−02 | 4.62E−02 |
| Sarcalumenin | SRL | Q86TD4 | 0.71 | 0.13 | 1.29 | 1.64E−02 | 3.76E−02 |
| Malonyl-CoA decarboxylase, mitochondrial | MLYCD | O95822 | 0.71 | 0.29 | 1.12 | 8.59E−04 | 2.70E−03 |
| ATPase ASNA1 | ASNA1 | O43681 | 0.71 | 0.35 | 1.08 | 1.10E−04 | 4.58E−04 |
| Interleukin-12 subunit beta | IL12B | P29460 | 0.71 | 0.35 | 1.06 | 9.13E−05 | 3.90E−04 |
| Gamma-interferon-inducible protein 16: Isoform | IFI16 | Q16666 | 0.71 | 0.47 | 0.95 | 1.03E−08 | 1.54E−07 |
| 2, Hematopoietic expression, interferon-inducible | | | | | | | |
| nature, and nuclear localization 1 | | | | | | | |
| Anion exchange transporter | SLC26A7 | Q8TE54 | 0.71 | 0.24 | 1.18 | 3.08E−03 | 8.45E−03 |
| Mortality factor 4-like protein 1 | MORF4L1 | Q9UBU8 | 0.71 | 0.25 | 1.16 | 2.58E−03 | 7.20E−03 |
| C-C motif chemokine 27 | CCL27 | Q9Y4X3 | 0.71 | 0.2 | 1.23 | 6.72E−03 | 1.71E−02 |
| C-X-C motif chemokine 11 | CXCL11 | O14625 | 0.71 | 0.51 | 0.91 | 1.44E−12 | 4.52E−11 |
| Carbonic anhydrase 3 | CA3 | P07451 | 0.71 | 0.43 | 0.98 | 5.23E−07 | 5.19E−06 |
| Lamin-B1 | LMNB1 | P20700 | 0.71 | 0.23 | 1.2 | 3.61E−03 | 9.76E−03 |
| L-lactate dehydrogenase B chain | LDHB | P07195 | 0.71 | 0.44 | 0.97 | 1.58E−07 | 1.83E−06 |
| Interferon lambda-1 | IFNL1 | Q8IU54 | 0.71 | 0.37 | 1.04 | 4.25E−05 | 2.04E−04 |
| cAMP-regulated phosphoprotein 19 | ARPP19 | P56211 | 0.71 | 0.22 | 1.19 | 4.51E−03 | 1.19E−02 |
| Cathepsin Z | CTSZ | Q9UBR2 | 0.71 | 0.18 | 1.24 | 8.91E−03 | 2.18E−02 |
| Heat shock 70 kDa protein 1A | PCSK2 | P0DMV8 | 0.71 | 0.21 | 1.21 | 5.51E−03 | 1.43E−02 |
| ALK and LTK ligand 2 | FAM150B | Q6UX46 | 0.71 | 0.47 | 0.95 | 5.82E−09 | 9.31E−08 |
| Intercellular adhesion molecule 4 | ICAM4 | Q14773 | 0.71 | 0.35 | 1.06 | 8.84E−05 | 3.80E−04 |
| Membrane-associated progesterone receptor | PGRMC1 | O00264 | 0.71 | 0.42 | 1.01 | 2.00E−06 | 1.62E−05 |
| component 1 | | | | | | | |
| Serine protease inhibitor Kazal-type 14 | SPINK14 | Q6IE38 | 0.71 | 0.11 | 1.31 | 1.98E−02 | 4.43E−02 |
| Galectin-9 | LGALS9 | O00182 | 0.71 | 0.14 | 1.29 | 1.53E−02 | 3.53E−02 |
| Poly(U)-binding-splicing factor PUF60 | PUF60 | Q9UHX1 | 0.7 | 0.17 | 1.23 | 9.59E−03 | 2.33E−02 |
| Heat shock 70 kDa protein 1A | HSPA1A | P0DMV8 | 0.7 | 0.17 | 1.24 | 1.01E−02 | 2.43E−02 |
| EF-hand calcium-binding domain-containing | EFCAB14 | O75071 | 0.7 | 0.19 | 1.22 | 7.55E−03 | 1.88E−02 |
| protein 14: N-term | | | | | | | |
| Serine/threonine-protein kinase VRK1 | VRK1 | Q99986 | 0.7 | 0.12 | 1.29 | 1.79E−02 | 4.04E−02 |
| Regulatory factor X-associated protein | RFXAP | O00287 | 0.7 | 0.13 | 1.27 | 1.67E−02 | 3.81E−02 |
| Diphosphoinositol polyphosphate | NUDT3 | O95989 | 0.7 | 0.4 | 0.99 | 3.92E−06 | 2.84E−05 |
| phosphohydrolase 1 | | | | | | | |
| Amyloid beta A4 precursor protein-binding | APBB 1 | O00213 | 0.7 | 0.23 | 1.18 | 3.76E−03 | 1.01E−02 |
| family B member 1: Phosphotyrosine Interaction | | | | | | | |
| Domain 1 | | | | | | | |
| Collagen type IV alpha-3-binding protein: StAR- | COL4A3BP | Q9Y5P4 | 0.7 | 0.49 | 0.91 | 4.45E−11 | 1.08E−09 |
| related lipid-transfer domain, isoform 2 | | | | | | | |
| Prolyl endopeptidase | PREP | P48147 | 0.7 | 0.45 | 0.94 | 2.01E−08 | 2.79E−07 |
| Rho-related GTP-binding protein RhoE | RND3 | P61587 | 0.7 | 0.13 | 1.27 | 1.55E−02 | 3.57E−02 |
| Probable tRNA(His) guanylyltransferase | THG1L | Q9NWX6 | 0.7 | 0.4 | 1 | 5.71E−06 | 3.91E−05 |
| AP-1 complex subunit sigma-2 | AP1S2 | P56377 | 0.7 | 0.34 | 1.05 | 1.15E−04 | 4.76E−04 |
| Density-regulated protein | DENR | O43583 | 0.7 | 0.37 | 1.03 | 3.34E−05 | 1.66E−04 |
| Ephrin-B3: Extracellular domain | EFNB3 | Q15768 | 0.7 | 0.36 | 1.04 | 6.29E−05 | 2.83E−04 |
| Caspase-2 | CASP2 | P42575 | 0.7 | 0.19 | 1.22 | 7.08E−03 | 1.78E−02 |
| Baculoviral IAP repeat-containing protein 3 | BIRC3 | Q13489 | 0.7 | 0.23 | 1.18 | 3.75E−03 | 1.01E−02 |
| Gremlin-2 | GREM2 | Q9H772 | 0.7 | 0.19 | 1.22 | 7.10E−03 | 1.79E−02 |
| NFU1 iron-sulfur cluster scaffold homolog, | NFU1 | Q9UMS0 | 0.7 | 0.41 | 1 | 2.84E−06 | 2.16E−05 |
| mitochondrial | | | | | | | |
| DnaJ homolog subfamily B member 12 | DNAJB12 | Q9NXW2 | 0.7 | 0.4 | 1 | 6.05E−06 | 4.08E−05 |
| UPF0606 protein KIAA1549L | KIAA1549L | Q6ZVL6 | 0.69 | 0.12 | 1.27 | 1.76E−02 | 3.99E−02 |
| Heat shock 70 kDa protein 1A | HSPA1A | P0DMV8 | 0.69 | 0.18 | 1.19 | 8.44E−03 | 2.08E−02 |
| DnaJ homolog subfamily B member 2 | DNAJB2 | P25686 | 0.69 | 0.43 | 0.96 | 4.16E−07 | 4.33E−06 |
| SH3 and multiple ankyrin repeat domains protein | SHANK3 | Q9BYB0 | 0.69 | 0.43 | 0.95 | 1.74E−07 | 1.99E−06 |
| 3 | | | | | | | |
| Defensin-5 | DEFA5 | Q01523 | 0.69 | 0.38 | 0.99 | 7.87E−06 | 5.09E−05 |
| Geranylgeranyl pyrophosphate synthase | GGPS1 | O95749 | 0.69 | 0.28 | 1.1 | 9.09E−04 | 2.84E−03 |
| Protein LZIC | LZIC | Q8WZA0 | 0.69 | 0.26 | 1.12 | 1.71E−03 | 4.95E−03 |
| CD5 antigen-like | CD5L | O43866 | 0.69 | 0.41 | 0.98 | 1.48E−06 | 1.25E−05 |
| Interleukin-24 | IL24 | Q13007 | 0.69 | 0.37 | 1.02 | 3.45E−05 | 1.71E−04 |
| Casein kinase II subunit alpha | CSNK2A1 | P68400 | 0.69 | 0.32 | 1.07 | 2.74E−04 | 1.00E−03 |
| Desert hedgehog protein N-product | DHH | O43323 | 0.69 | 0.42 | 0.96 | 5.46E−07 | 5.39E−06 |
| Collagen alpha-1(XXIII) chain | COL23A1 | Q86Y22 | 0.69 | 0.38 | 0.99 | 1.07E−05 | 6.47E−05 |
| Galectin-3-binding protein | LGALS3BP | Q08380 | 0.69 | 0.39 | 0.99 | 6.77E−06 | 4.50E−05 |
| Heat shock 70 kDa protein 1A | HSPA1A | P0DMV8 | 0.69 | 0.17 | 1.21 | 9.08E−03 | 2.22E−02 |
| N-terminal pro-BNP | NPPB | P16860 | 0.69 | 0.52 | 0.85 | 2.03E−15 | 9.61E−14 |
| Stromal cell-derived factor 1 | CXCL12 | P48061 | 0.69 | 0.12 | 1.25 | 1.68E−02 | 3.83E−02 |
| Multimerin-2 | MMRN2 | Q9H8L6 | 0.69 | 0.23 | 1.15 | 3.10E−03 | 8.51E−03 |
| CXADR-like membrane protein: Extracellular | CLMP | Q9H6B4 | 0.68 | 0.29 | 1.06 | 5.61E−04 | 1.86E−03 |
| domain | | | | | | | |
| Transmembrane protein 190 | TMEM190 | Q8WZ59 | 0.68 | 0.35 | 1.01 | 5.08E−05 | 2.36E−04 |
| Protein FAM163B | FAM163B | P0C2L3 | 0.68 | 0.27 | 1.09 | 1.12E−03 | 3.43E−03 |
| HemK methyltransferase family member 2 | N6AMT1 | Q9Y5N5 | 0.68 | 0.37 | 0.99 | 1.85E−05 | 1.02E−04 |
| ZW10 interactor | ZWINT | O95229 | 0.68 | 0.17 | 1.19 | 8.88E−03 | 2.18E−02 |
| Junctional adhesion molecule C | JAM3 | Q9BX67 | 0.68 | 0.43 | 0.92 | 4.63E−08 | 5.97E−07 |
| Fibroblast growth factor 6 | FGF6 | P10767 | 0.68 | 0.38 | 0.97 | 6.96E−06 | 4.60E−05 |
| Cathepsin D | CTSD | P07339 | 0.68 | 0.37 | 0.99 | 1.51E−05 | 8.61E−05 |
| GDP-fucose protein O-fucosyltransferase 1 | POFUT1 | Q9H488 | 0.68 | 0.31 | 1.05 | 3.20E−04 | 1.14E−03 |
| Ethanolamine kinase 1 | ETNK1 | Q9HBU6 | 0.68 | 0.12 | 1.24 | 1.77E−02 | 4.00E−02 |
| Calcineurin B homologous protein 3 | TESC | Q96BS2 | 0.67 | 0.34 | 1 | 5.98E−05 | 2.72E−04 |
| Neuroendocrine convertase 1 | PCSK1 | P29120 | 0.67 | 0.12 | 1.21 | 1.60E−02 | 3.67E−02 |
| 26S proteasome non-ATPase regulatory subunit 9 | PSMD9 | O00233 | 0.67 | 0.31 | 1.03 | 2.34E−04 | 8.74E−04 |
| Methyl-CpG-binding domain protein 1 | MBD1 | Q9UIS9 | 0.67 | 0.31 | 1.04 | 2.95E−04 | 1.06E−03 |
| Vascular endothelial growth factor D | FIGF | O43915 | 0.67 | 0.32 | 1.03 | 2.03E−04 | 7.73E−04 |
| Interleukin-15 | IL15 | P40933 | 0.67 | 0.29 | 1.05 | 6.38E−04 | 2.08E−03 |
| Growth/differentiation factor 5 | GDF5 | P43026 | 0.67 | 0.11 | 1.23 | 1.99E−02 | 4.44E−02 |
| Kynureninase | KYNU | Q16719 | 0.67 | 0.13 | 1.22 | 1.57E−02 | 3.61E−02 |
| Iron-sulfur cluster assembly enzyme ISCU, | ISCU | Q9H1K1 | 0.67 | 0.3 | 1.04 | 3.77E−04 | 1.31E−03 |
| mitochondrial | | | | | | | |
| NADH dehydrogenase [ubiquinone] flavoprotein | NDUFV2 | P19404 | 0.67 | 0.35 | 1 | 4.64E−05 | 2.20E−04 |
| 2, mitochondrial | | | | | | | |
| BRISC complex subunit Abrol | FAM175B | Q15018 | 0.67 | 0.37 | 0.98 | 1.32E−05 | 7.64E−05 |
| Neuromedin-B | NMB | P08949 | 0.67 | 0.16 | 1.19 | 1.03E−02 | 2.48E−02 |
| Syntaxin-8 | STX8 | Q9UNK0 | 0.66 | 0.44 | 0.88 | 7.28E−09 | 1.14E−07 |
| Homeobox protein DLX-3 | DLX3 | O60479 | 0.66 | 0.27 | 1.06 | 1.03E−03 | 3.16E−03 |
| Secreted frizzled-related protein 4 | SFRP4 | Q6FHJ7 | 0.66 | 0.22 | 1.11 | 3.42E−03 | 9.31E−03 |
| Calsequestrin-2 | CASQ2 | O14958 | 0.66 | 0.15 | 1.17 | 1.05E−02 | 2.53E−02 |
| Isoform L-VEGF165 | VEGFA | P15692 | 0.66 | 0.32 | 0.99 | 1.12E−04 | 4.67E−04 |
| Ubiquitin + 1, truncated mutation for UbB | RPS27A | P62979 | 0.66 | 0.43 | 0.89 | 1.53E−08 | 2.22E−07 |
| C-X-C motif chemokine 5 | CXCL5 | P42830 | 0.66 | 0.16 | 1.16 | 9.82E−03 | 2.37E−02 |
| Adapter molecule crk | CRK | P46108 | 0.66 | 0.43 | 0.88 | 9.98E−09 | 1.51E−07 |
| Heterogeneous nuclear ribonucleoproteins A2/B1 | HNRNPA2B1 | P22626 | 0.66 | 0.42 | 0.9 | 7.56E−08 | 9.19E−07 |
| Tumor necrosis factor ligand superfamily | TNFSF14 | O43557 | 0.66 | 0.15 | 1.17 | 1.05E−02 | 2.53E−02 |
| member 14 | | | | | | | |
| 40S ribosomal protein S4, X isoform | RPS4X | P62701 | 0.66 | 0.46 | 0.86 | 2.25E−10 | 4.76E−09 |
| Probable RNA-binding protein 23 | RBM23 | Q86U06 | 0.65 | 0.29 | 1.01 | 4.15E−04 | 1.43E−03 |
| Protein tyrosine phosphatase type IVA 2 | PTP4A2 | Q12974 | 0.65 | 0.4 | 0.91 | 6.53E−07 | 6.32E−06 |
| Ras-related protein Rab-18 | RAB18 | Q9NP72 | 0.65 | 0.19 | 1.11 | 5.46E−03 | 1.42E−02 |
| Retinoblastoma-binding protein 5 | RBBP5 | Q15291 | 0.65 | 0.11 | 1.2 | 1.81E−02 | 4.09E−02 |
| Protein TSSC4 | TSSC4 | Q9Y5U2 | 0.65 | 0.43 | 0.88 | 1.69E−08 | 2.42E−07 |
| Nidogen-1 | NID1 | P14543 | 0.65 | 0.19 | 1.11 | 5.45E−03 | 1.42E−02 |
| Scavenger receptor cysteine-rich type 1 protein | CD163 | Q86VB7 | 0.65 | 0.26 | 1.05 | 1.08E−03 | 3.32E−03 |
| M130 | | | | | | | |
| Protein S100-A9 | S100A9 | P06702 | 0.65 | 0.41 | 0.89 | 1.14E−07 | 1.35E−06 |
| Peptidyl-prolyl cis-trans isomerase FKBP14 | FKBP14 | Q9NWM8 | 0.65 | 0.41 | 0.88 | 4.73E−08 | 6.09E−07 |
| Guanine nucleotide exchange factor VAV3 | VAV3 | Q9UKW4 | 0.65 | 0.21 | 1.09 | 3.92E−03 | 1.05E−02 |
| Interleukin-23 | IL12B IL23A | Q9NPF7\|P29460 | 0.64 | 0.26 | 1.01 | 9.48E−04 | 2.95E−03 |
| Malignant T-cell-amplified sequence 1 | MCTS1 | Q9ULC4 | 0.64 | 0.27 | 1 | 5.84E−04 | 1.92E−03 |
| Tax1-binding protein 3 | TAX1BP3 | O14907 | 0.64 | 0.37 | 0.9 | 2.72E−06 | 2.09E−05 |
| Thiosulfate sulfurtransferase | TST | Q16762 | 0.64 | 0.36 | 0.91 | 6.49E−06 | 4.33E−05 |
| Vacuolar protein sorting-associated protein 4B | VPS4B | O75351 | 0.64 | 0.21 | 1.07 | 3.39E−03 | 9.23E−03 |
| Glycerophosphocholine phosphodiesterase | GPCPD1 | Q9NPB8 | 0.64 | 0.34 | 0.93 | 2.31E−05 | 1.22E−04 |
| GPCPD1 | | | | | | | |
| Hexokinase-2 | HK2 | P52789 | 0.64 | 0.37 | 0.91 | 4.62E−06 | 3.26E−05 |
| C-C motif chemokine 8 | CCL8 | P80075 | 0.64 | 0.26 | 1.01 | 9.85E−04 | 3.06E−03 |
| L-lactate dehydrogenase A chain | LDHA | P00338 | 0.64 | 0.3 | 0.97 | 1.94E−04 | 7.41E−04 |
| Isopentenyl-diphosphate Delta-isomerase 1 | IDI1 | Q13907 | 0.64 | 0.42 | 0.85 | 8.45E−09 | 1.30E−07 |
| Quinone oxidoreductase PIG3 | TP53I3 | Q53FA7 | 0.64 | 0.38 | 0.89 | 7.00E−07 | 6.57E−06 |
| Nuclear transport factor 2 | NUTF2 | P61970 | 0.64 | 0.27 | 1.02 | 7.39E−04 | 2.37E−03 |
| Haloacid dehalogenase-like hydrolase domain- | HDHD3 | Q9BSH5 | 0.64 | 0.29 | 0.98 | 3.11E−04 | 1.11E−03 |
| containing protein 3 | | | | | | | |
| Fibroblast growth factor 18 | FGF18 | O76093 | 0.64 | 0.1 | 1.19 | 2.07E−02 | 4.60E−02 |
| High affinity nerve growth factor receptor | NTRK1 | P04629 | 0.64 | 0.17 | 1.1 | 7.13E−03 | 1.79E−02 |
| Interleukin-11 | IL11 | P20809 | 0.64 | 0.19 | 1.1 | 5.54E−03 | 1.44E−02 |
| Testican-1 | SPOCK1 | Q08629 | 0.64 | 0.25 | 1.04 | 1.40E−03 | 4.17E−03 |
| Titin | TTN | Q8WZ42 | 0.63 | 0.22 | 1.04 | 2.37E−03 | 6.68E−03 |
| ATP-dependent DNA helicase Q1 | RECQL | P46063 | 0.63 | 0.37 | 0.88 | 1.54E−06 | 1.29E−05 |
| Coiled-coil domain-containing protein 50 | CCDC50 | Q8IVM0 | 0.63 | 0.4 | 0.86 | 1.25E−07 | 1.47E−06 |
| Heterogeneous nuclear ribonucleoprotein A1 | HNRNPA1 | P09651 | 0.63 | 0.43 | 0.83 | 6.80E−10 | 1.33E−08 |
| Thioredoxin reductase 1, cytoplasmic | TXNRD1 | Q16881 | 0.63 | 0.4 | 0.87 | 8.41E−08 | 1.02E−06 |
| Dolichyl-diphosphooligosaccharide--protein | DDOST | P39656 | 0.63 | 0.33 | 0.93 | 3.64E−05 | 1.79E−04 |
| glycosyltransferase 48 kDa subunit | | | | | | | |
| Protocadherin gamma-A10 | PCDHGA10 | Q9Y5H3 | 0.63 | 0.27 | 1 | 6.16E−04 | 2.02E−03 |
| Metalloproteinase inhibitor 4 | TIMP4 | Q99727 | 0.63 | 0.22 | 1.03 | 2.51E−03 | 7.03E−03 |
| Serine protease inhibitor Kazal-type 8 | SPINK8 | P0C7L1 | 0.63 | 0.33 | 0.92 | 2.69E−05 | 1.39E−04 |
| Glycodelin | PAEP | P09466 | 0.63 | 0.11 | 1.15 | 1.78E−02 | 4.01E−02 |
| Phosphoribosyl pyrophosphate synthase- | PRPSAP1 | Q14558 | 0.63 | 0.3 | 0.96 | 2.17E−04 | 8.19E−04 |
| associated protein 1 | | | | | | | |
| Glutathione S-transferase Mu 3 | GSTM3 | P21266 | 0.63 | 0.3 | 0.97 | 2.03E−04 | 7.74E−04 |
| SLP adapter and CSK-interacting membrane | SCIMP | Q6UWF3 | 0.62 | 0.17 | 1.07 | 6.89E−03 | 1.74E−02 |
| protein | | | | | | | |
| Cellular retinoic acid-binding protein 2 | CRABP2 | P29373 | 0.62 | 0.26 | 0.97 | 7.14E−04 | 2.30E−03 |
| Lethal(3)malignant brain tumor-like protein 2 | L3MBTL2 | Q969R5 | 0.62 | 0.38 | 0.86 | 4.73E−07 | 4.84E−06 |
| Sorting nexin-17 | SNX17 | Q15036 | 0.62 | 0.2 | 1.03 | 3.41E−03 | 9.28E−03 |
| Selenide, water dikinase 1 | SEPHS1 | P49903 | 0.62 | 0.37 | 0.88 | 1.55E−06 | 1.29E−05 |
| DnaJ homolog subfamily C member 17 | DNAJC17 | Q9NVM6 | 0.62 | 0.32 | 0.91 | 4.50E−05 | 2.14E−04 |
| Low-density lipoprotein receptor-related protein | LRP12 | Q9Y561 | 0.62 | 0.17 | 1.08 | 6.70E−03 | 1.71E−02 |
| 12 | | | | | | | |
| Rab GDP dissociation inhibitor alpha | GDI1 | P31150 | 0.62 | 0.39 | 0.85 | 1.28E−07 | 1.50E−06 |
| Folate receptor alpha | FOLR1 | P15328 | 0.62 | 0.21 | 1.04 | 3.19E−03 | 8.74E−03 |
| 39S ribosomal protein L2, mitochondrial | MRPL2 | Q5T653 | 0.62 | 0.18 | 1.06 | 5.81E−03 | 1.50E−02 |
| Nicotinamide N-methyltransferase | NNMT | P40261 | 0.62 | 0.29 | 0.95 | 2.40E−04 | 8.93E−04 |
| Heme oxygenase 2 | HMOX2 | P30519 | 0.62 | 0.23 | 1.01 | 1.85E−03 | 5.34E−03 |
| Superoxide dismutase [Cu—Zn] | SOD1 | P00441 | 0.62 | 0.3 | 0.94 | 1.33E−04 | 5.37E−04 |
| Allograft inflammatory factor 1 | AIF1 | P55008 | 0.62 | 0.21 | 1.03 | 2.83E−03 | 7.83E−03 |
| Kremen protein 2 | KREMEN2 | Q8NCW0 | 0.62 | 0.25 | 1 | 1.14E−03 | 3.47E−03 |
| Ubiquitin-conjugating enzyme E2 L3 | UBE2L3 | P68036 | 0.62 | 0.38 | 0.86 | 4.97E−07 | 5.02E−06 |
| Death-associated protein kinase 2 | DAPK2 | Q9UIK4 | 0.62 | 0.29 | 0.94 | 1.91E−04 | 7.30E−04 |
| 40S ribosomal protein S3 | RPS3 | P23396 | 0.62 | 0.26 | 0.98 | 7.75E−04 | 2.46E−03 |
| cGMP-inhibited 3′,5′-cyclic phosphodiesterase A | PDE3A | Q14432 | 0.62 | 0.13 | 1.12 | 1.40E−02 | 3.25E−02 |
| Protein FAM19A5 | FAM19A5 | Q7Z5A7 | 0.62 | 0.1 | 1.14 | 1.89E−02 | 4.25E−02 |
| Protein FAM171A1 | FAM171A1 | Q5VUB5 | 0.62 | 0.21 | 1.03 | 2.91E−03 | 8.04E−03 |
| WSC domain-containing protein 2 | WSCD2 | Q2TBF2 | 0.62 | 0.37 | 0.87 | 1.27E−06 | 1.09E−05 |
| Kallikrein-15 | KLK15 | Q9H2R5 | 0.62 | 0.29 | 0.96 | 2.74E−04 | 1.00E−03 |
| Procollagen-lysine, 2-oxoglutarate 5-dioxygenase | PLOD2 | O00469 | 0.62 | 0.3 | 0.95 | 1.83E−04 | 7.04E−04 |
| 2 | | | | | | | |
| Heparan sulfate glucosamine 3-O-sulfotransferase | HS3ST3A1 | Q9Y663 | 0.62 | 0.37 | 0.87 | 8.68E−07 | 7.90E−06 |
| 3A1 | | | | | | | |
| Trefoil factor 2 | TFF2 | Q03403 | 0.62 | 0.31 | 0.92 | 6.74E−05 | 3.02E−04 |
| Torsin-1A-interacting protein 1: Perinuclear | TOR1AIP1 | Q5JTV8 | 0.61 | 0.38 | 0.85 | 2.90E−07 | 3.16E−06 |
| domain | | | | | | | |
| Voltage-dependent L-type calcium channel | CACNB4 | O00305 | 0.61 | 0.38 | 0.85 | 4.51E−07 | 4.64E−06 |
| subunit beta-4 | | | | | | | |
| Peptide chain release factor 1-like, mitochondrial | MTRF1L | Q9UGC7 | 0.61 | 0.31 | 0.91 | 7.83E−05 | 3.42E−04 |
| DnaJ homolog subfamily B member 9 | DNAJB9 | Q9UBS3 | 0.61 | 0.19 | 1.03 | 4.65E−03 | 1.22E−02 |
| Coiled-coil-helix-coiled-coil-helix domain- | CHCHD10 | Q8WYQ3 | 0.61 | 0.17 | 1.04 | 6.39E−03 | 1.63E−02 |
| containing protein 10, mitochondrial | | | | | | | |
| 72 kDa inositol polyphosphate 5-phosphatase | INPP5E | Q9NRR6 | 0.61 | 0.1 | 1.12 | 1.84E−02 | 4.15E−02 |
| UV excision repair protein RAD23 homolog B | RAD23B | P54727 | 0.61 | 0.37 | 0.86 | 8.97E−07 | 8.11E−06 |
| GRB2-related adapter protein | GRAP | Q13588 | 0.61 | 0.31 | 0.92 | 7.87E−05 | 3.44E−04 |
| Rap1 GTPase-GDP dissociation stimulator 1 | RAP1GDS1 | P52306 | 0.61 | 0.26 | 0.96 | 5.54E−04 | 1.84E−03 |
| Cytokine receptor-like factor 1 | CRLF1 | O75462 | 0.61 | 0.23 | 0.99 | 1.69E−03 | 4.90E−03 |
| Calcineurin subunit B type 1 | PPP3R1 | P63098 | 0.61 | 0.24 | 0.97 | 1.21E−03 | 3.67E−03 |
| TATA-binding protein-associated factor 2N | TAF15 | Q92804 | 0.61 | 0.36 | 0.85 | 1.23E−06 | 1.07E−05 |
| Small nuclear ribonucleoprotein Sm D2 | SNRPD2 | P62316 | 0.61 | 0.29 | 0.93 | 2.13E−04 | 8.06E−04 |
| Protein S100-Z | S100Z | Q8WXG8 | 0.61 | 0.24 | 0.99 | 1.41E−03 | 4.19E−03 |
| C-C motif chemokine 28 | CCL28 | Q9NRJ3 | 0.61 | 0.32 | 0.9 | 3.47E−05 | 1.72E−04 |
| WNT1-inducible-signaling pathway protein 1 | WISP1 | O95388 | 0.61 | 0.13 | 1.09 | 1.29E−02 | 3.02E−02 |
| Chitotriosidase-1 | CHIT1 | Q13231 | 0.61 | 0.39 | 0.83 | 3.48E−08 | 4.61E−07 |
| Peroxiredoxin-1 | PRDX1 | Q06830 | 0.61 | 0.3 | 0.91 | 8.85E−05 | 3.80E−04 |
| Single-pass membrane and coiled-coil domain- | SMCO2 | A6NFE2 | 0.61 | 0.17 | 1.05 | 6.17E−03 | 1.58E−02 |
| containing protein 2 | | | | | | | |
| Amyloid-like protein 2 | APLP2 | Q06481 | 0.6 | 0.27 | 0.93 | 3.96E−04 | 1.37E−03 |
| Sorcin | SRI | P30626 | 0.6 | 0.32 | 0.88 | 3.00E−05 | 1.52E−04 |
| Proteasome assembly chaperone 3 | PSMG3 | Q9BT73 | 0.6 | 0.37 | 0.84 | 3.33E−07 | 3.60E−06 |
| Serine/threonine-protein kinase B-raf | BRAF | P15056 | 0.6 | 0.21 | 0.98 | 2.58E−03 | 7.21E−03 |
| NAD(P)H-hydrate epimerase | APOA1BP | Q8NCW5 | 0.6 | 0.28 | 0.92 | 2.61E−04 | 9.61E−04 |
| Ubiquitin carboxyl-terminal hydrolase 15 | USP15 | Q9Y4E8 | 0.6 | 0.29 | 0.91 | 1.28E−04 | 5.19E−04 |
| Small nuclear ribonucleoprotein G | SNRPG | P62308 | 0.6 | 0.37 | 0.83 | 3.81E−07 | 4.03E−06 |
| Heterogeneous nuclear ribonucleoprotein K | HNRNPK | P61978 | 0.6 | 0.38 | 0.82 | 9.40E−08 | 1.13E−06 |
| Protein kinase C zeta type | PRKCZ | Q05513 | 0.6 | 0.28 | 0.92 | 2.29E−04 | 8.58E−04 |
| Tenascin | TNC | P24821 | 0.6 | 0.14 | 1.07 | 1.13E−02 | 2.71E−02 |
| X-ray repair cross-complementing protein 6 | XRCC6 | P12956 | 0.6 | 0.28 | 0.92 | 2.31E−04 | 8.65E−04 |
| Arylsulfatase A | ARSA | P15289 | 0.6 | 0.12 | 1.09 | 1.44E−02 | 3.35E−02 |
| Bone morphogenetic protein 10 | BMP10 | O95393 | 0.6 | 0.15 | 1.06 | 9.75E−03 | 2.36E−02 |
| Inorganic pyrophosphatase | PPA1 | Q15181 | 0.6 | 0.26 | 0.94 | 5.19E−04 | 1.75E−03 |
| Non-receptor tyrosine-protein kinase TYK2 | TYK2 | P29597 | 0.6 | 0.22 | 0.97 | 2.05E−03 | 5.85E−03 |
| Elongation factor 1-beta | EEF1B2 | P24534 | 0.6 | 0.3 | 0.89 | 7.67E−05 | 3.37E−04 |
| Patatin-like phospholipase domain-containing | PNPLA2 | Q96AD5 | 0.6 | 0.09 | 1.12 | 2.21E−02 | 4.87E−02 |
| protein 2 | | | | | | | |
| Ubiquitin-protein ligase E3A | UBE3A | Q05086 | 0.59 | 0.33 | 0.84 | 5.40E−06 | 3.74E−05 |
| ADP-ribosylation factor-like protein 1 | ARL1 | P40616 | 0.59 | 0.1 | 1.08 | 1.77E−02 | 4.00E−02 |
| Twinfilin-1 | TWF1 | Q12792 | 0.59 | 0.33 | 0.84 | 5.79E−06 | 3.94E−05 |
| Mitogen-activated protein kinase kinase kinase 3 | MAP3K3 | Q99759 | 0.59 | 0.24 | 0.93 | 8.20E−04 | 2.59E−03 |
| Mitochondrial fission 1 protein | FIS1 | Q9Y3D6 | 0.59 | 0.2 | 0.98 | 3.27E−03 | 8.94E−03 |
| AMMECR1-like protein | AMMECR1L | Q6DCA0 | 0.59 | 0.24 | 0.93 | 8.28E−04 | 2.61E−03 |
| Calponin-2 | CNN2 | Q99439 | 0.59 | 0.28 | 0.9 | 2.02E−04 | 7.72E−04 |
| Complement receptor type 1 | CR1 | P17927 | 0.59 | 0.09 | 1.09 | 2.00E−02 | 4.46E−02 |
| Serine/threonine-protein kinase MRCK beta | CDC42BPB | Q9Y5S2 | 0.59 | 0.24 | 0.95 | 1.10E−03 | 3.39E−03 |
| Phosphatidylethanolamine-binding protein 1 | PEBP1 | P30086 | 0.59 | 0.37 | 0.82 | 1.96E−07 | 2.23E−06 |
| Asialoglycoprotein receptor 1 | ASGR1 | P07306 | 0.59 | 0.14 | 1.03 | 1.01E−02 | 2.43E−02 |
| Soluble calcium-activated nucleotidase 1 | CANT1 | Q8WVQ1 | 0.59 | 0.26 | 0.93 | 5.55E−04 | 1.84E−03 |
| Proenkephalin-A | PENK | P01210 | 0.59 | 0.29 | 0.9 | 1.31E−04 | 5.31E−04 |
| Ubiquitin-like modifier-activating enzyme ATG7 | ATG7 | O95352 | 0.58 | 0.27 | 0.9 | 3.17E−04 | 1.13E−03 |
| Oncostatin-M | OSM | P13725 | 0.58 | 0.16 | 1 | 6.36E−03 | 1.63E−02 |
| V-type immunoglobulin domain-containing | C10orf54 | Q9H7M9 | 0.58 | 0.23 | 0.93 | 9.99E−04 | 3.10E−03 |
| suppressor of T-cell activation: Extracellular | | | | | | | |
| domain | | | | | | | |
| Collagen alpha-1(X) chain | COL10A1 | Q03692 | 0.58 | 0.23 | 0.92 | 1.07E−03 | 3.28E−03 |
| R-spondin-1 | RSPO1 | Q2MKA7 | 0.58 | 0.2 | 0.96 | 2.85E−03 | 7.86E−03 |
| Ras-related protein Rab-2A | RAB2A | P61019 | 0.58 | 0.33 | 0.83 | 6.06E−06 | 4.08E−05 |
| Protein phosphatase methylesterase 1 | PPME1 | Q9Y570 | 0.58 | 0.32 | 0.85 | 1.44E−05 | 8.26E−05 |
| Uroporphyrinogen decarboxylase | UROD | P06132 | 0.58 | 0.25 | 0.91 | 6.57E−04 | 2.14E−03 |
| Vesicle-associated membrane protein 7 | VAMP7 | P51809 | 0.58 | 0.25 | 0.92 | 6.01E−04 | 1.97E−03 |
| Interleukin-18-binding protein | IL18BP | O95998 | 0.58 | 0.22 | 0.94 | 1.72E−03 | 4.98E−03 |
| Seizure 6-like protein 2 | SEZ6L2 | Q6UXD5 | 0.58 | 0.35 | 0.8 | 7.06E−07 | 6.59E−06 |
| Kinesin-like protein KIF23 | KIF23 | Q02241 | 0.58 | 0.34 | 0.83 | 2.72E−06 | 2.09E−05 |
| Calcium/calmodulin-dependent 3′,5′-cyclic | PDE1A | P54750 | 0.58 | 0.26 | 0.9 | 3.50E−04 | 1.23E−03 |
| nucleotide phosphodiesterase 1A | | | | | | | |
| Chordin-like protein 2 | CHRDL2 | Q6WN34 | 0.58 | 0.16 | 1.01 | 7.08E−03 | 1.78E−02 |
| Vitrin | VIT | Q6UXI7 | 0.58 | 0.16 | 1.01 | 7.13E−03 | 1.79E−02 |
| Dolichyl-diphosphooligosaccharide--protein | RPN1 | P04843 | 0.58 | 0.26 | 0.9 | 4.13E−04 | 1.42E−03 |
| glycosyltransferase subunit 1: Lumenal domain | | | | | | | |
| Protein FAM162A | FAM162A | Q96A26 | 0.58 | 0.21 | 0.94 | 1.92E−03 | 5.49E−03 |
| Oocyte-secreted protein 2 | OOSP2 | Q86WS3 | 0.58 | 0.14 | 1.02 | 9.26E−03 | 2.26E−02 |
| Mitogen-activated protein kinase kinase kinase | MAP4K1 | Q92918 | 0.58 | 0.27 | 0.9 | 2.49E−04 | 9.24E−04 |
| kinase 1 | | | | | | | |
| Ornithine decarboxylase antizyme 1 | OAZ1 | P54368 | 0.58 | 0.32 | 0.84 | 1.34E−05 | 7.77E−05 |
| Casein kinase I isoform gamma-2 | CSNK1G2 | P78368 | 0.57 | 0.26 | 0.87 | 3.27E−04 | 1.16E−03 |
| Ubiquitin-conjugating enzyme E2 D4 | UBE2D4 | Q9Y2X8 | 0.57 | 0.34 | 0.79 | 6.75E−07 | 6.44E−06 |
| Leukocyte cell-derived chemotaxin-2 | LECT2 | O14960 | 0.57 | 0.19 | 0.95 | 3.52E−03 | 9.54E−03 |
| BAG family molecular chaperone regulator 1 | BAG1 | Q99933 | 0.57 | 0.27 | 0.87 | 2.13E−04 | 8.07E−04 |
| Putative D-tyrosyl-tRNA(Tyr) deacylase 2 | DTD2 | Q96FN9 | 0.57 | 0.34 | 0.8 | 1.25E−06 | 1.07E−05 |
| Histone deacetylase 2 | HDAC2 | Q92769 | 0.57 | 0.36 | 0.79 | 1.67E−07 | 1.92E−06 |
| Ubiquitin-like domain-containing CTD | UBLCP1 | Q8WVY7 | 0.57 | 0.34 | 0.8 | 1.26E−06 | 1.09E−05 |
| phosphatase 1 | | | | | | | |
| Histone deacetylase 8 | HDAC8 | Q9BY41 | 0.57 | 0.09 | 1.05 | 1.95E−02 | 4.37E−02 |
| Stem cell growth factor-beta | CLEC11A | Q9Y240 | 0.57 | 0.16 | 0.99 | 7.28E−03 | 1.82E−02 |
| Megakaryocyte-associated tyrosine-protein | MATK | P42679 | 0.57 | 0.12 | 1.02 | 1.25E−02 | 2.96E−02 |
| kinase | | | | | | | |
| Alcohol dehydrogenase [NADP(+)] | AKR1A1 | P14550 | 0.57 | 0.35 | 0.79 | 5.05E−07 | 5.07E−06 |
| Ubiquitin carboxyl-terminal hydrolase isozyme | UCHL1 | P09936 | 0.57 | 0.26 | 0.88 | 3.31E−04 | 1.17E−03 |
| L1 | | | | | | | |
| HLA class II histocompatibility antigen, DQ | HLA-DQA2 | P01906 | 0.57 | 0.08 | 1.06 | 2.27E−02 | 4.98E−02 |
| alpha 2 chain | | | | | | | |
| Disks large homolog 4 | DLG4 | P78352 | 0.57 | 0.28 | 0.86 | 1.28E−04 | 5.19E−04 |
| Lymphocyte activation gene 3 protein | LAG3 | P18627 | 0.57 | 0.18 | 0.95 | 3.93E−03 | 1.05E−02 |
| Forkhead box protein O3 | FOXO3 | O43524 | 0.56 | 0.34 | 0.78 | 9.75E−07 | 8.68E−06 |
| Charged multivesicular body protein 3 | VPS24 | Q9Y3E7 | 0.56 | 0.34 | 0.79 | 6.67E−07 | 6.41E−06 |
| Oligoribonuclease, mitochondrial | REXO2 | Q9Y3B8 | 0.56 | 0.3 | 0.81 | 2.10E−05 | 1.12E−04 |
| Sialic acid synthase | NANS | Q9NR45 | 0.56 | 0.29 | 0.82 | 4.83E−05 | 2.27E−04 |
| Ras-related protein Rab-1A | RAB1A | P62820 | 0.56 | 0.3 | 0.81 | 1.53E−05 | 8.69E−05 |
| Eukaryotic translation initiation factor 4E | EIF4E | P06730 | 0.56 | 0.31 | 0.81 | 1.11E−05 | 6.71E−05 |
| Ras GTPase-activating protein-binding protein 1 | G3BP1 | Q13283 | 0.56 | 0.36 | 0.76 | 3.85E−08 | 5.00E−07 |
| Amphiregulin | AREG | P15514 | 0.56 | 0.35 | 0.78 | 2.66E−07 | 2.95E−06 |
| Resistin | RETN | Q9HD89 | 0.56 | 0.21 | 0.91 | 1.58E−03 | 4.64E−03 |
| Epidermal growth factor receptor substrate 15- | EPS15L1 | Q9UBC2 | 0.56 | 0.33 | 0.79 | 1.94E−06 | 1.57E−05 |
| like 1 | | | | | | | |
| Fibroblast growth factor 16 | FGF16 | O43320 | 0.56 | 0.35 | 0.77 | 1.69E−07 | 1.95E−06 |
| Inosine-5′-monophosphate dehydrogenase 1 | IMPDH1 | P20839 | 0.56 | 0.33 | 0.79 | 2.21E−06 | 1.77E−05 |
| SHC-transforming protein 1: Src Homology | SHC1 | P29353 | 0.56 | 0.36 | 0.75 | 1.76E−08 | 2.50E−07 |
| domain | | | | | | | |
| Gastrokine-2 | GKN2 | Q86XP6 | 0.56 | 0.26 | 0.85 | 2.53E−04 | 9.34E−04 |
| Probable inactive ribonuclease-like protein 13 | RNASE13 | Q5GAN3 | 0.56 | 0.18 | 0.95 | 4.22E−03 | 1.12E−02 |
| 14-3-3 protein theta | YWHAQ | P27348 | 0.56 | 0.22 | 0.89 | 1.02E−03 | 3.15E−03 |
| SH3 domain-binding protein 2 | SH3BP2 | P78314 | 0.56 | 0.22 | 0.89 | 1.18E−03 | 3.57E−03 |
| Heterogeneous nuclear ribonucleoprotein F | HNRNPF | P52597 | 0.56 | 0.33 | 0.79 | 1.58E−06 | 1.31E−05 |
| Mucosal addressin cell adhesion molecule 1 | MADCAM1 | Q13477 | 0.55 | 0.17 | 0.93 | 4.29E−03 | 1.14E−02 |
| Tapasin | TAPBP | O15533 | 0.55 | 0.3 | 0.8 | 1.64E−05 | 9.19E−05 |
| Protein kinase C and casein kinase substrate in | PACSIN1 | Q9BY11 | 0.55 | 0.29 | 0.8 | 3.33E−05 | 1.66E−04 |
| neurons protein 1 | | | | | | | |
| Ubiquilin-4 | UBQLN4 | Q9NRR5 | 0.55 | 0.36 | 0.75 | 3.62E−08 | 4.74E−07 |
| Heterogeneous nuclear ribonucleoprotein M | HNRNPM | P52272 | 0.55 | 0.18 | 0.92 | 3.55E−03 | 9.63E−03 |
| Keratin, type I cytoskeletal 20 | KRT20 | P35900 | 0.55 | 0.23 | 0.88 | 7.49E−04 | 2.39E−03 |
| Kallikrein-14 | KLK14 | Q9P0G3 | 0.55 | 0.21 | 0.88 | 1.42E−03 | 4.19E−03 |
| Cyclin-dependent kinase 16 | PCTK1 | Q00536 | 0.55 | 0.3 | 0.8 | 1.44E−05 | 8.24E−05 |
| Protein S100-A8/A9 heterodimer | S100A8 S100A9 | P06702\|P05109 | 0.55 | 0.3 | 0.81 | 1.50E−05 | 8.55E−05 |
| Dehydrogenase/reductase SDR family member 9 | DHRS9 | Q9BPW9 | 0.55 | 0.17 | 0.92 | 4.32E−03 | 1.15E−02 |
| UMP-CMP kinase | CMPK1 | P30085 | 0.55 | 0.25 | 0.84 | 3.29E−04 | 1.16E−03 |
| Dynactin subunit 6 | DCTN6 | O00399 | 0.55 | 0.32 | 0.79 | 4.61E−06 | 3.26E−05 |
| Chromobox protein homolog 1 | CBX1 | P83916 | 0.55 | 0.28 | 0.82 | 6.69E−05 | 3.00E−04 |
| Urokinase plasminogen activator surface receptor | PLAUR | Q03405 | 0.55 | 0.13 | 0.98 | 1.13E−02 | 2.69E−02 |
| Tyrosine-protein kinase Lck | LCK | P06239 | 0.55 | 0.17 | 0.93 | 4.70E−03 | 1.24E−02 |
| Malate dehydrogenase, cytoplasmic | MDH1 | P40925 | 0.55 | 0.3 | 0.8 | 1.29E−05 | 7.50E−05 |
| Hsp90 co-chaperone Cdc37 | CDC37 | Q16543 | 0.55 | 0.29 | 0.81 | 2.74E−05 | 1.41E−04 |
| Interleukin-17 receptor C | IL17RC | Q8NAC3 | 0.55 | 0.14 | 0.96 | 8.76E−03 | 2.15E−02 |
| Procollagen galactosyltransferase 1 | COLGALT1 | Q8NBJ5 | 0.55 | 0.2 | 0.89 | 1.80E−03 | 5.19E−03 |
| Acid ceramidase | ASAH1 | Q13510 | 0.55 | 0.33 | 0.78 | 8.94E−07 | 8.09E−06 |
| Polyadenylate-binding protein 2 | PABPN1 | Q86U42 | 0.55 | 0.3 | 0.81 | 2.13E−05 | 1.14E−04 |
| Platelet-derived growth factor D | PDGFD | Q9GZP0 | 0.55 | 0.23 | 0.87 | 6.82E−04 | 2.20E−03 |
| Angiopoietin-related protein 3 | ANGPTL3 | Q9Y5C1 | 0.54 | 0.11 | 0.96 | 1.34E−02 | 3.14E−02 |
| Ribosome-recycling factor, mitochondrial | MRRF | Q96E11 | 0.54 | 0.14 | 0.94 | 8.37E−03 | 2.07E−02 |
| Transcriptional activator protein Pur-alpha | PURA | Q00577 | 0.54 | 0.31 | 0.77 | 3.47E−06 | 2.55E−05 |
| AP-2 complex subunit alpha-2 | AP2A2 | O94973 | 0.54 | 0.33 | 0.75 | 6.96E−07 | 6.56E−06 |
| Ataxin-3 | ATXN3 | P54252 | 0.54 | 0.28 | 0.8 | 5.38E−05 | 2.47E−04 |
| TATA box-binding protein-like protein 1 | TBPL1 | P62380 | 0.54 | 0.25 | 0.82 | 2.13E−04 | 8.06E−04 |
| Ubiquilin-2 | UBQLN2 | Q9UHD9 | 0.54 | 0.33 | 0.74 | 5.22E−07 | 5.19E−06 |
| STAM-binding protein | STAMBP | O95630 | 0.54 | 0.29 | 0.8 | 2.99E−05 | 1.52E−04 |
| Cytosolic 5′-nucleotidase 3A | NT5C3A | Q9H0P0 | 0.54 | 0.25 | 0.82 | 2.35E−04 | 8.79E−04 |
| Myeloperoxidase | MPO | P05164 | 0.54 | 0.16 | 0.92 | 5.55E−03 | 1.44E−02 |
| Connective tissue growth factor | CTGF | P29279 | 0.54 | 0.23 | 0.85 | 5.92E−04 | 1.95E−03 |
| Hyaluronan and proteoglycan link protein 1 | HAPLN1 | P10915 | 0.54 | 0.1 | 0.98 | 1.58E−02 | 3.61E−02 |
| Leukotriene A-4 hydrolase | LTA4H | P09960 | 0.54 | 0.23 | 0.86 | 6.17E−04 | 2.02E−03 |
| Calcium/calmodulin-dependent protein kinase | CAMK2A | Q9UQM7 | 0.54 | 0.22 | 0.87 | 1.15E−03 | 3.50E−03 |
| type II subunit alpha | | | | | | | |
| Integrin alpha-I: beta-1 complex | ITGA1 ITGB1 | P05556\|P56199 | 0.54 | 0.28 | 0.8 | 5.31E−05 | 2.44E−04 |
| Interleukin-25 | IL25 | Q9H293 | 0.54 | 0.12 | 0.97 | 1.24E−02 | 2.93E−02 |
| P-selectin | SELP | P16109 | 0.54 | 0.26 | 0.82 | 1.71E−04 | 6.64E−04 |
| Stem cell growth factor-alpha | CLEC11A | Q9Y240 | 0.54 | 0.17 | 0.91 | 4.07E−03 | 1.09E−02 |
| Glycylpeptide N-tetradecanoyltransferase 1 | NMT1 | P30419 | 0.54 | 0.3 | 0.78 | 1.05E−05 | 6.42E−05 |
| Ras GTPase-activating protein 1 | RASA1 | P20936 | 0.54 | 0.25 | 0.83 | 3.14E−04 | 1.12E−03 |
| Tumor necrosis factor receptor superfamily | TNFRSF10B | O14763 | 0.54 | 0.09 | 0.99 | 1.85E−02 | 4.17E−02 |
| member 10B: Extracellular domain, TNFR-Cys | | | | | | | |
| domains 1-3 | | | | | | | |
| Lysozyme-like protein 2 | LYZL2 | Q7Z4W2 | 0.54 | 0.17 | 0.91 | 4.38E−03 | 1.16E−02 |
| Endoplasmin | HSP90B1 | P14625 | 0.54 | 0.27 | 0.8 | 7.79E−05 | 3.41E−04 |
| Low-density lipoprotein receptor class A domain- | LDLRAD4 | O15165 | 0.54 | 0.26 | 0.83 | 1.77E−04 | 6.84E−04 |
| containing protein 4 | | | | | | | |
| Hepatitis A virus cellular receptor 1 | HAVCR1 | Q96D42 | 0.54 | 0.29 | 0.79 | 2.81E−05 | 1.44E−04 |
| Cullin-3 | CUL3 | Q13618 | 0.53 | 0.19 | 0.87 | 2.41E−03 | 6.78E−03 |
| Retinoic acid receptor responder protein 3 | RARRES3 | Q9UL19 | 0.53 | 0.14 | 0.92 | 8.30E−03 | 2.05E−02 |
| Microtubule-associated proteins 1A/1B light | MAP1LC3B | Q9GZQ8 | 0.53 | 0.3 | 0.76 | 5.07E−06 | 3.53E−05 |
| chain 3B | | | | | | | |
| Gamma-interferon-inducible protein 16: Isoform | IFI16 | Q16666 | 0.53 | 0.24 | 0.82 | 3.57E−04 | 1.25E−03 |
| 2, Hematopoietic expression, interferon-inducible | | | | | | | |
| nature, and nuclear localization 2 | | | | | | | |
| Collagen type IV alpha-3-binding protein: PH | COL4A3BP | Q9Y5P4 | 0.53 | 0.3 | 0.77 | 8.21E−06 | 5.25E−05 |
| domain | | | | | | | |
| NAD kinase | NADK | O95544 | 0.53 | 0.09 | 0.97 | 1.80E−02 | 4.07E−02 |
| Inhibin beta B chain | INHBB | P09529 | 0.53 | 0.21 | 0.85 | 1.32E−03 | 3.94E−03 |
| Coiled-coil domain-containing protein 134 | CCDC134 | Q9H6E4 | 0.53 | 0.31 | 0.76 | 2.24E−06 | 1.79E−05 |
| Death domain-containing protein CRADD | CRADD | P78560 | 0.53 | 0.19 | 0.88 | 2.57E−03 | 7.19E−03 |
| Guanylate kinase | GUK1 | Q16774 | 0.53 | 0.29 | 0.78 | 1.47E−05 | 8.40E−05 |
| Ran-specific GTPase-activating protein | RANBP1 | P43487 | 0.53 | 0.27 | 0.78 | 5.52E−05 | 2.53E−04 |
| Calpain small subunit 1 | CAPNS1 | P04632 | 0.53 | 0.28 | 0.78 | 3.37E−05 | 1.67E−04 |
| UBX domain-containing protein 2B | UBXN2B | Q14CS0 | 0.53 | 0.26 | 0.79 | 1.13E−04 | 4.69E−04 |
| Prostaglandin E synthase 2 | PTGES2 | Q9H7Z7 | 0.53 | 0.28 | 0.78 | 2.79E−05 | 1.43E−04 |
| Eukaryotic initiation factor 4A-I | EIF4A1 | P60842 | 0.53 | 0.35 | 0.71 | 6.11E−09 | 9.71E−08 |
| Adenosine kinase | ADK | P55263 | 0.53 | 0.19 | 0.87 | 1.95E−03 | 5.57E−03 |
| Guanine nucleotide-binding protein G(i) subunit | GNAI1 | P63096 | 0.53 | 0.21 | 0.85 | 1.02E−03 | 3.15E−03 |
| alpha-1 | | | | | | | |
| SUMO-conjugating enzyme UBC9 | UBE2I | P63279 | 0.53 | 0.31 | 0.76 | 3.95E−06 | 2.85E−05 |
| Growth-regulated alpha protein | CXCL1 | P09341 | 0.53 | 0.29 | 0.78 | 1.42E−05 | 8.18E−05 |
| Ubiquitin-fold modifier-conjugating enzyme 1 | UFC1 | Q9Y3C8 | 0.53 | 0.29 | 0.77 | 1.13E−05 | 6.79E−05 |
| Mitogen-activated protein kinase 8 | MAPK8 | P45983 | 0.53 | 0.29 | 0.77 | 1.34E−05 | 7.75E−05 |
| Ubiquitin-conjugating enzyme E2 N | UBE2N | P61088 | 0.53 | 0.23 | 0.84 | 6.27E−04 | 2.05E−03 |
| Fc receptor-like protein 3 | FCRL3 | Q96P31 | 0.53 | 0.09 | 0.96 | 1.85E−02 | 4.17E−02 |
| WNT1-inducible-signaling pathway protein 2 | WISP2 | O76076 | 0.53 | 0.12 | 0.94 | 1.10E−02 | 2.62E−02 |
| RAF proto-oncogene serine/threonine-protein | RAF1 | P04049 | 0.52 | 0.17 | 0.87 | 3.43E−03 | 9.33E−03 |
| kinase | | | | | | | |
| DnaJ homolog subfamily A member 2 | DNAJA2 | O60884 | 0.52 | 0.24 | 0.81 | 3.30E−04 | 1.17E−03 |
| Ubiquitin-conjugating enzyme E2 R1 | CDC34 | P49427 | 0.52 | 0.18 | 0.87 | 2.99E−03 | 8.24E−03 |
| Polyadenylate-binding protein 4 | PABPC4 | Q13310 | 0.52 | 0.25 | 0.79 | 1.46E−04 | 5.80E−04 |
| Tumor necrosis factor alpha-induced protein 3 | TNFAIP3 | P21580 | 0.52 | 0.25 | 0.8 | 1.74E−04 | 6.77E−04 |
| Cysteine--tRNA ligase, cytoplasmic | CARS | P49589 | 0.52 | 0.27 | 0.77 | 5.31E−05 | 2.44E−04 |
| Glycylpeptide N-tetradecanoyltransferase 2 | NMT2 | O60551 | 0.52 | 0.32 | 0.71 | 1.29E−07 | 1.50E−06 |
| Heat shock 70 kDa protein 1B | HSPA1B | P0DMV9 | 0.52 | 0.08 | 0.97 | 2.16E−02 | 4.78E−02 |
| Pre-mRNA-splicing factor SYF2 | SYF2 | O95926 | 0.52 | 0.29 | 0.75 | 9.78E−06 | 6.07E−05 |
| Prostaglandin reductase 1 | PTGR1 | Q14914 | 0.52 | 0.29 | 0.75 | 8.15E−06 | 5.23E−05 |
| Cytokine receptor common subunit gamma | IL2RG | P31785 | 0.52 | 0.32 | 0.72 | 5.80E−07 | 5.68E−06 |
| Myeloid cell surface antigen CD33 | CD33 | P20138 | 0.52 | 0.23 | 0.82 | 4.94E−04 | 1.68E−03 |
| Dual specificity protein phosphatase 3 | DUSP3 | P51452 | 0.52 | 0.29 | 0.75 | 1.16E−05 | 6.93E−05 |
| Membrane metallo-endopeptidase-like 1 | MMEL1 | Q495T6 | 0.52 | 0.09 | 0.95 | 1.68E−02 | 3.83E−02 |
| Transforming growth factor beta-2 | TGFB2 | P61812 | 0.52 | 0.13 | 0.92 | 9.37E−03 | 2.28E−02 |
| Eukaryotic translation initiation factor 5A-1 | EIF5A | P63241 | 0.52 | 0.33 | 0.7 | 5.58E−08 | 6.98E−07 |
| Proteasome activator complex subunit 1 | PSME1 | Q06323 | 0.52 | 0.22 | 0.81 | 6.11E−04 | 2.00E−03 |
| Sorting nexin-1 | SNX1 | Q13596 | 0.52 | 0.14 | 0.89 | 7.23E−03 | 1.81E−02 |
| Fragile X mental retardation protein 1 | FMR1 | Q06787 | 0.52 | 0.28 | 0.76 | 2.07E−05 | 1.11E−04 |
| DnaJ homolog subfamily C member 30 | DNAJC30 | Q96LL9 | 0.52 | 0.19 | 0.85 | 1.92E−03 | 5.49E−03 |
| Fc receptor-like protein 4: Cytoplasmic domain | FCRL4 | Q96PJ5 | 0.52 | 0.27 | 0.77 | 3.92E−05 | 1.91E−04 |
| Leukocyte elastase inhibitor | SERPINB1 | P30740 | 0.51 | 0.24 | 0.79 | 2.97E−04 | 1.07E−03 |
| Peptidyl-prolyl cis-trans isomerase FKBP1B | FKBP1B | P68106 | 0.51 | 0.3 | 0.72 | 2.33E−06 | 1.85E−05 |
| General vesicular transport factor p115 | USO1 | O60763 | 0.51 | 0.27 | 0.75 | 3.21E−05 | 1.61E−04 |
| Small ubiquitin-related modifier 3 | SUMO3 | P55854 | 0.51 | 0.33 | 0.69 | 3.40E−08 | 4.52E−07 |
| Brorin | VWC2 | Q2TAL6 | 0.51 | 0.11 | 0.92 | 1.30E−02 | 3.05E−02 |
| Cadherin-17 | CDH17 | Q12864 | 0.51 | 0.25 | 0.77 | 1.24E−04 | 5.07E−04 |
| Ubiquitin-conjugating enzyme E2 variant 2 | UBE2V2 | Q15819 | 0.51 | 0.29 | 0.74 | 7.77E−06 | 5.03E−05 |
| Ubiquitin | RPS27A | P62979 | 0.51 | 0.1 | 0.92 | 1.39E−02 | 3.24E−02 |
| C-C motif chemokine 19 | CCL19 | Q99731 | 0.51 | 0.2 | 0.82 | 1.39E−03 | 4.13E−03 |
| Focal adhesion kinase 1 | PTK2 | Q05397 | 0.51 | 0.14 | 0.87 | 7.13E−03 | 1.79E−02 |
| Protein FAM172A | FAM172A | Q8WUF8 | 0.51 | 0.1 | 0.92 | 1.51E−02 | 3.50E−02 |
| Beta-defensin 121 | DEFB121 | Q5J5C9 | 0.51 | 0.25 | 0.78 | 1.56E−04 | 6.14E−04 |
| Interleukin-17 receptor B | IL17RB | Q9NRM6 | 0.51 | 0.31 | 0.71 | 9.08E−07 | 8.16E−06 |
| BRICHOS domain-containing protein 5 | BRICD5 | Q6PL45 | 0.51 | 0.26 | 0.76 | 6.96E−05 | 3.10E−04 |
| ERO1-like protein beta | ERO1LB | Q86YB8 | 0.51 | 0.19 | 0.82 | 1.55E−03 | 4.55E−03 |
| Microfibrillar-associated protein 3-like | MFAP3L | O75121 | 0.51 | 0.11 | 0.9 | 1.14E−02 | 2.71E−02 |
| Lactoylglutathione lyase | GLO1 | Q04760 | 0.51 | 0.22 | 0.8 | 5.14E−04 | 1.73E−03 |
| Ribosome-binding protein 1 | RRBP1 | Q9P2E9 | 0.51 | 0.2 | 0.82 | 1.34E−03 | 4.00E−03 |
| Rho GTPase-activating protein 25 | ARHGAP25 | P42331 | 0.5 | 0.3 | 0.69 | 3.70E−07 | 3.94E−06 |
| E3 ubiquitin-protein ligase CHIP | STUB1 | Q9UNE7 | 0.5 | 0.26 | 0.74 | 4.80E−05 | 2.26E−04 |
| Ribosomal protein S6 kinase alpha-6 | RPS6KA6 | Q9UK32 | 0.5 | 0.26 | 0.73 | 3.26E−05 | 1.63E−04 |
| Ephrin-A2 | EFNA2 | O43921 | 0.5 | 0.07 | 0.93 | 2.16E−02 | 4.76E−02 |
| Mitogen-activated protein kinase 9 | MAPK9 | P45984 | 0.5 | 0.26 | 0.74 | 3.57E−05 | 1.75E−04 |
| Osteoclast-stimulating factor 1 | OSTF1 | Q92882 | 0.5 | 0.12 | 0.87 | 9.67E−03 | 2.35E−02 |
| Activated RNA polymerase II transcriptional | SUB1 | P53999 | 0.5 | 0.24 | 0.76 | 2.05E−04 | 7.80E−04 |
| coactivator p15 | | | | | | | |
| Vacuolar protein sorting-associated protein 26A | VPS26A | O75436 | 0.5 | 0.3 | 0.7 | 8.84E−07 | 8.01E−06 |
| Plexin-A4 | PLXNA4 | Q9HCM2 | 0.5 | 0.22 | 0.78 | 3.85E−04 | 1.34E−03 |
| Rab GDP dissociation inhibitor beta | GDI2 | P50395 | 0.5 | 0.28 | 0.72 | 8.55E−06 | 5.43E−05 |
| Mitogen-activated protein kinase 1 | MAPK1 | P28482 | 0.5 | 0.3 | 0.7 | 1.28E−06 | 1.09E−05 |
| Protein kinase C iota type | PRKCI | P41743 | 0.5 | 0.3 | 0.7 | 9.84E−07 | 8.75E−06 |
| Peptidyl-prolyl cis-trans isomerase E | PPIE | Q9UNP9 | 0.5 | 0.3 | 0.69 | 4.97E−07 | 5.02E−06 |
| Lon protease homolog, mitochondrial | LONP1 | P36776 | 0.5 | 0.32 | 0.68 | 4.58E−08 | 5.92E−07 |
| Protocadherin gamma-A2 | PCDHGA2 | Q9Y5H1 | 0.5 | 0.21 | 0.8 | 7.92E−04 | 2.51E−03 |
| Potassium-transporting ATPase subunit beta | ATP4B | P51164 | 0.5 | 0.08 | 0.92 | 1.98E−02 | 4.42E−02 |
| Syntaxin-4 | STX4 | Q12846 | 0.49 | 0.3 | 0.68 | 6.58E−07 | 6.35E−06 |
| Eukaryotic translation initiation factor 3 subunit | EIF3G | O75821 | 0.49 | 0.32 | 0.66 | 1.63E−08 | 2.36E−07 |
| G | | | | | | | |
| Mothers against decapentaplegic homolog 4 | SMAD4 | Q13485 | 0.49 | 0.25 | 0.73 | 6.49E−05 | 2.92E−04 |
| Complement factor H-related protein 2 | CFHR2 | P36980 | 0.49 | 0.18 | 0.8 | 2.09E−03 | 5.94E−03 |
| Axin interactor, dorsalization-associated protein | AIDA | Q96BJ3 | 0.49 | 0.24 | 0.73 | 1.19E−04 | 4.87E−04 |
| Glia maturation factor beta | GMFB | P60983 | 0.49 | 0.22 | 0.75 | 3.57E−04 | 1.25E−03 |
| 7-methylguanosine phosphate-specific 5′- | NT5C3L | Q969T7 | 0.49 | 0.16 | 0.82 | 3.93E−03 | 1.06E−02 |
| nucleotidase | | | | | | | |
| Proliferating cell nuclear antigen | PCNA | P12004 | 0.49 | 0.24 | 0.73 | 1.19E−04 | 4.90E−04 |
| C-C motif chemokine 25 | CCL25 | O15444 | 0.49 | 0.24 | 0.75 | 1.68E−04 | 6.55E−04 |
| Peptide YY | PYY | P10082 | 0.49 | 0.14 | 0.85 | 6.74E−03 | 1.71E−02 |
| CDK5 and ABL1 enzyme substrate 2 | CABLES2 | Q9BTV7 | 0.49 | 0.08 | 0.9 | 1.89E−02 | 4.25E−02 |
| Syntaxin-7 | STX7 | O15400 | 0.49 | 0.26 | 0.71 | 2.03E−05 | 1.09E−04 |
| DnaJ homolog subfamily C member 10 | DNAJC10 | Q8IXB1 | 0.49 | 0.23 | 0.74 | 1.76E−04 | 6.83E−04 |
| Oxytocin-neurophysin 1 | OXT | P01178 | 0.49 | 0.12 | 0.86 | 9.85E−03 | 2.38E−02 |
| Breast cancer anti-estrogen resistance protein | BCAR3 | O75815 | 0.48 | 0.13 | 0.83 | 7.15E−03 | 1.79E−02 |
| 3: Guanine Nucleotide Exchange Factor Domain | | | | | | | |
| Protein argonaute-3 | AGO3 | Q9H9G7 | 0.48 | 0.22 | 0.73 | 2.76E−04 | 1.01E−03 |
| Inhibin beta C chain | INHBC | P55103 | 0.48 | 0.24 | 0.71 | 8.05E−05 | 3.51E−04 |
| Epidermal growth factor-like protein 6 | EGFL6 | Q8IUX8 | 0.48 | 0.2 | 0.76 | 8.89E−04 | 2.79E−03 |
| N-myc-interactor | NMI | Q13287 | 0.48 | 0.25 | 0.72 | 4.75E−05 | 2.24E−04 |
| G antigen 2A | GAGE2A | Q6NT46 | 0.48 | 0.08 | 0.87 | 1.77E−02 | 4.00E−02 |
| Peroxisomal 2,4-dienoyl-CoA reductase | DECR2 | Q9NUI1 | 0.48 | 0.28 | 0.68 | 3.99E−06 | 2.88E−05 |
| Methionine aminopeptidase 2 | METAP2 | P50579 | 0.48 | 0.29 | 0.67 | 8.04E−07 | 7.36E−06 |
| Tyrosine-protein phosphatase non-receptor type | PTPN11 | Q06124 | 0.48 | 0.19 | 0.76 | 1.05E−03 | 3.22E−03 |
| 11 | | | | | | | |
| N-acetyl-D-glucosamine kinase | NAGK | Q9UJ70 | 0.48 | 0.17 | 0.78 | 2.20E−03 | 6.24E−03 |
| Regulator of microtubule dynamics protein 1 | RMDN1 | Q96DB5 | 0.48 | 0.29 | 0.67 | 6.74E−07 | 6.44E−06 |
| Heat shock 70 kDa protein 1A | HSPA1A | P0DMV8 | 0.48 | 0.08 | 0.87 | 1.72E−02 | 3.90E−02 |
| Ribonucleoside-diphosphate reductase subunit | RRM2B | Q7LG56 | 0.48 | 0.24 | 0.72 | 7.74E−05 | 3.39E−04 |
| M2 B | | | | | | | |
| Heterogeneous nuclear ribonucleoprotein D-like | HNRNPDL | O14979 | 0.47 | 0.31 | 0.64 | 2.02E−08 | 2.80E−07 |
| Double-strand break repair protein MRE11 | MRE11A | P49959 | 0.47 | 0.13 | 0.81 | 6.26E−03 | 1.60E−02 |
| Ubiquitin-conjugating enzyme E2 variant 1 | UBE2V1 | Q13404 | 0.47 | 0.27 | 0.67 | 3.86E−06 | 2.81E−05 |
| MOB kinase activator 1A | MOB1A | Q9H8S9 | 0.47 | 0.17 | 0.77 | 1.93E−03 | 5.52E−03 |
| Retinal rod rhodopsin-sensitive cGMP 3′,5′-cyclic | PDE6D | O43924 | 0.47 | 0.19 | 0.76 | 1.01E−03 | 3.14E−03 |
| phosphodiesterase subunit delta | | | | | | | |
| RNA polymerase II subunit A C-terminal domain | SSU72 | Q9NP77 | 0.47 | 0.21 | 0.72 | 3.05E−04 | 1.09E−03 |
| phosphatase SSU72 | | | | | | | |
| Vigilin | HDLBP | Q00341 | 0.47 | 0.18 | 0.76 | 1.42E−03 | 4.21E−03 |
| Death-associated protein kinase 1 | DAPK1 | P53355 | 0.47 | 0.19 | 0.76 | 1.27E−03 | 3.82E−03 |
| Vesicle-fusing ATPase | NSF | P46459 | 0.47 | 0.21 | 0.72 | 4.34E−04 | 1.49E−03 |
| Guanylate-binding protein 1 | GBP1 | P32455 | 0.47 | 0.09 | 0.84 | 1.49E−02 | 3.44E−02 |
| CASP8 and FADD-like apoptosis regulator | CFLAR | O15519 | 0.47 | 0.24 | 0.7 | 5.52E−05 | 2.53E−04 |
| Ras-related protein Rab-4A | RAB4A | P20338 | 0.47 | 0.26 | 0.68 | 1.03E−05 | 6.30E−05 |
| T-cell leukemia/lymphoma protein 1A | TCL1A | P56279 | 0.47 | 0.14 | 0.81 | 6.00E−03 | 1.54E−02 |
| Ubiquitin-conjugating enzyme E2 S | UBE2S | Q16763 | 0.47 | 0.22 | 0.72 | 2.42E−04 | 8.99E−04 |
| Arginase-2, mitochondrial | ARG2 | P78540 | 0.47 | 0.1 | 0.83 | 1.27E−02 | 3.00E−02 |
| Protein MEMO1 | MEMO1 | Q9Y316 | 0.47 | 0.2 | 0.73 | 5.54E−04 | 1.84E−03 |
| Hydroxyacylglutathione hydrolase, mitochondrial | HAGH | Q16775 | 0.47 | 0.14 | 0.8 | 5.75E−03 | 1.49E−02 |
| High mobility group protein HMGI-C | HMGA2 | P52926 | 0.47 | 0.13 | 0.82 | 7.19E−03 | 1.80E−02 |
| PIK3CA/PIK3R1 | PIK3CA PIK3R1 | P27986\|P42336 | 0.47 | 0.2 | 0.73 | 4.86E−04 | 1.65E−03 |
| Cystatin-D | CST5 | P28325 | 0.47 | 0.24 | 0.7 | 6.63E−05 | 2.98E−04 |
| Phosphatidylinositol 3,4,5-trisphosphate 3- | PTEN | P60484 | 0.47 | 0.26 | 0.68 | 1.07E−05 | 6.48E−05 |
| phosphatase and dual-specificity protein | | | | | | | |
| phosphatase PTEN | | | | | | | |
| Heat shock protein HSP 90-beta | HSP90AB1 | P08238 | 0.47 | 0.27 | 0.66 | 2.25E−06 | 1.79E−05 |
| ETS domain-containing protein Elk-3 | ELK3 | P41970 | 0.47 | 0.3 | 0.64 | 6.97E−08 | 8.52E−07 |
| Beta-1,3-glucosyltransferase | B3GALTL | Q6Y288 | 0.47 | 0.18 | 0.75 | 1.43E−03 | 4.24E−03 |
| Bc12-associated agonist of cell death | BAD | Q92934 | 0.47 | 0.29 | 0.64 | 1.79E−07 | 2.05E−06 |
| WAP four-disulfide core domain protein 3 | WFDC3 | Q8IUB2 | 0.47 | 0.28 | 0.65 | 6.80E−07 | 6.45E−06 |
| R-spondin-2 | RSPO2 | Q6UXX9 | 0.47 | 0.08 | 0.85 | 1.66E−02 | 3.80E−02 |
| Golgi membrane protein 1 | GOLM1 | Q8NBJ4 | 0.47 | 0.16 | 0.77 | 2.63E−03 | 7.33E−03 |
| Peptidyl-prolyl cis-trans isomerase FKBP2 | FKBP2 | P26885 | 0.47 | 0.25 | 0.69 | 2.42E−05 | 1.27E−04 |
| Isochorismatase domain-containing protein 1 | ISOC1 | Q96CN7 | 0.47 | 0.2 | 0.73 | 5.11E−04 | 1.72E−03 |
| Ras GTPase-activating protein-binding protein 2 | G3BP2 | Q9UN86 | 0.47 | 0.31 | 0.63 | 1.16E−08 | 1.72E−07 |
| Phospholipase D3 | PLD3 | Q8IV08 | 0.46 | 0.27 | 0.66 | 4.35E−06 | 3.10E−05 |
| Thrombospondin type-1 domain-containing | THSD7A | Q9UPZ6 | 0.46 | 0.17 | 0.74 | 2.02E−03 | 5.75E−03 |
| protein 7A: Thrombospondin type-1 domain 6 | | | | | | | |
| Proteasome subunit alpha type-7 | PSMA7 | O14818 | 0.46 | 0.19 | 0.73 | 9.79E−04 | 3.04E−03 |
| Endothelin-2 | EDN2 | P20800 | 0.46 | 0.14 | 0.79 | 5.28E−03 | 1.38E−02 |
| p53 and DNA damage-regulated protein 1 | PDRG1 | Q9NUG6 | 0.46 | 0.24 | 0.69 | 5.93E−05 | 2.70E−04 |
| Cell cycle checkpoint protein RAD1 | RAD1 | O60671 | 0.46 | 0.06 | 0.85 | 2.24E−02 | 4.93E−02 |
| PR domain zinc finger protein 4 | PRDM4 | Q9UKN5 | 0.46 | 0.16 | 0.76 | 2.61E−03 | 7.26E−03 |
| Syntaxin-10 | STX10 | O60499 | 0.46 | 0.12 | 0.79 | 7.49E−03 | 1.87E−02 |
| Cas scaffolding protein family member 4 | CASS4 | Q9NQ75 | 0.46 | 0.13 | 0.8 | 6.73E−03 | 1.71E−02 |
| Protein FAM210A | FAM210A | Q96ND0 | 0.46 | 0.07 | 0.86 | 2.20E−02 | 4.85E−02 |
| Ras-related C3 botulinum toxin substrate 3 | RAC3 | P60763 | 0.46 | 0.24 | 0.68 | 5.25E−05 | 2.43E−04 |
| NEDD8-conjugating enzyme UBE2F | UBE2F | Q969M7 | 0.46 | 0.18 | 0.75 | 1.50E−03 | 4.40E−03 |
| Ubiquitin carboxyl-terminal hydrolase 14 | USP14 | P54578 | 0.46 | 0.23 | 0.7 | 1.32E−04 | 5.35E−04 |
| Eukaryotic translation initiation factor 1A, Y- | EIF1AY | O14602 | 0.46 | 0.25 | 0.66 | 1.22E−05 | 7.18E−05 |
| chromosomal | | | | | | | |
| Glial fibrillary acidic protein | GFAP | P14136 | 0.46 | 0.1 | 0.81 | 1.20E−02 | 2.85E−02 |
| Mediator of RNA polymerase II transcription | MED1 | Q15648 | 0.46 | 0.09 | 0.82 | 1.41E−02 | 3.29E−02 |
| subunit 1 | | | | | | | |
| Pyridoxal phosphate phosphatase | PDXP | Q96GD0 | 0.46 | 0.27 | 0.64 | 9.11E−07 | 8.17E−06 |
| Proliferation-associated protein 2G4 | PA2G4 | Q9UQ80 | 0.46 | 0.25 | 0.68 | 2.60E−05 | 1.35E−04 |
| Complement C3b | C3 | P01024 | 0.46 | 0.28 | 0.63 | 2.26E−07 | 2.53E−06 |
| Alpha-1,3-mannosyl-glycoprotein 4-beta-N- | MGAT4C | Q9UBM8 | 0.46 | 0.08 | 0.84 | 1.75E−02 | 3.96E−02 |
| acetylglucosaminyltransferase C | | | | | | | |
| Nuclear migration protein nudC: CS domain | NUDC | Q9Y266 | 0.46 | 0.27 | 0.66 | 2.94E−06 | 2.22E−05 |
| Trimeric intracellular cation channel type B | TMEM38B | Q9NVV0 | 0.46 | 0.2 | 0.72 | 6.05E−04 | 1.99E−03 |
| Laminin subunit gamma-2 | LAMC2 | Q13753 | 0.46 | 0.08 | 0.85 | 1.80E−02 | 4.06E−02 |
| Unconventional myosin-VI | MYO6 | Q9UM54 | 0.46 | 0.18 | 0.74 | 1.31E−03 | 3.94E−03 |
| 1-phosphatidylinositol 4,5-bisphosphate | PLCG2 | P16885 | 0.45 | 0.24 | 0.66 | 1.80E−05 | 9.95E−05 |
| phosphodiesterase gamma-2 | | | | | | | |
| Immunoglobulin-binding protein 1 | IGBP1 | P78318 | 0.45 | 0.26 | 0.65 | 6.80E−06 | 4.52E−05 |
| ADP-ribosylation factor-like protein 3 | ARL3 | P36405 | 0.45 | 0.26 | 0.64 | 2.86E−06 | 2.17E−05 |
| Protein flightless-1 homolog | FLII | Q13045 | 0.45 | 0.25 | 0.65 | 7.23E−06 | 4.74E−05 |
| Cold-inducible RNA-binding protein | CIRBP | Q14011 | 0.45 | 0.29 | 0.61 | 5.10E−08 | 6.47E−07 |
| Differentially expressed in FDCP 6 homolog | DEF6 | Q9H4E7 | 0.45 | 0.23 | 0.66 | 5.03E−05 | 2.34E−04 |
| Triggering receptor expressed on myeloid cells 2 | TREM2 | Q9NZC2 | 0.45 | 0.18 | 0.73 | 1.33E−03 | 3.98E−03 |
| Proteasome activator complex subunit 2 | PSME2 | Q9UL46 | 0.45 | 0.21 | 0.69 | 2.28E−04 | 8.55E−04 |
| Peptidyl-prolyl cis-trans isomerase FKBP3 | FKBP3 | Q00688 | 0.45 | 0.28 | 0.62 | 2.72E−07 | 3.00E−06 |
| Heat shock protein beta-6 | HSPB6 | O14558 | 0.45 | 0.11 | 0.78 | 9.20E−03 | 2.25E−02 |
| Methenyltetrahydrofolate synthase domain- | MTHFSD | Q2M296 | 0.45 | 0.24 | 0.67 | 4.33E−05 | 2.08E−04 |
| containing protein | | | | | | | |
| Calcium/calmodulin-dependent protein kinase | CAMK2D | Q13557 | 0.45 | 0.23 | 0.68 | 8.62E−05 | 3.72E−04 |
| type II subunit delta | | | | | | | |
| Peptidyl-prolyl cis-trans isomerase A | PPIA | P62937 | 0.45 | 0.26 | 0.65 | 6.39E−06 | 4.28E−05 |
| Cytoplasmic protein NCK1 | NCK1 | P16333 | 0.45 | 0.07 | 0.83 | 2.06E−02 | 4.59E−02 |
| GrpE protein homolog 1, mitochondrial | GRPEL1 | Q9HAV7 | 0.45 | 0.27 | 0.64 | 2.31E−06 | 1.84E−05 |
| Vesicle transport through interaction with t- | VTI1A | Q96AJ9 | 0.45 | 0.18 | 0.72 | 1.13E−03 | 3.46E−03 |
| SNAREs homolog 1A | | | | | | | |
| VIP36-like protein | LMAN2L | Q9H0V9 | 0.45 | 0.25 | 0.64 | 7.73E−06 | 5.02E−05 |
| RING finger protein 215: Cytoplasmic domain | RNF215 | Q9Y6U7 | 0.45 | 0.2 | 0.71 | 5.37E−04 | 1.80E−03 |
| Forkhead box protein M1 | FOXM1 | Q08050 | 0.44 | 0.28 | 0.6 | 6.20E−08 | 7.64E−07 |
| Translation initiation factor eIF-2B subunit alpha | EIF2B1 | Q14232 | 0.44 | 0.28 | 0.59 | 2.26E−08 | 3.10E−07 |
| Signal transducer and activator of transcription 1- | STAT1 | P42224 | 0.44 | 0.29 | 0.59 | 8.56E−09 | 1.31E−07 |
| alpha/beta | | | | | | | |
| Spartin | SPG20 | Q8N0X7 | 0.44 | 0.22 | 0.67 | 1.28E−04 | 5.19E−04 |
| Cytosolic purine 5′-nucleotidase | NT5C2 | P49902 | 0.44 | 0.23 | 0.65 | 3.33E−05 | 1.66E−04 |
| Biliverdin reductase A | BLVRA | P53004 | 0.44 | 0.24 | 0.64 | 1.95E−05 | 1.06E−04 |
| Eukaryotic translation initiation factor 3 subunit J | EIF3J | O75822 | 0.44 | 0.25 | 0.62 | 5.51E−06 | 3.80E−05 |
| Inhibin beta A chain | INHBA | P08476 | 0.44 | 0.07 | 0.82 | 2.04E−02 | 4.54E−02 |
| Talin-2 | TLN2 | Q9Y4G6 | 0.44 | 0.17 | 0.71 | 1.62E−03 | 4.72E−03 |
| Glutathione S-transferase Mu 1 | GSTM1 | P09488 | 0.44 | 0.13 | 0.74 | 5.58E−03 | 1.44E−02 |
| Glutathione peroxidase 1 | GPX1 | P07203 | 0.44 | 0.25 | 0.64 | 1.26E−05 | 7.36E−05 |
| Early activation antigen CD69 | CD69 | Q07108 | 0.44 | 0.22 | 0.66 | 1.06E−04 | 4.45E−04 |
| Synaptotagmin-4 | SYT4 | Q9H2B2 | 0.44 | 0.19 | 0.69 | 6.46E−04 | 2.11E−03 |
| Ubiquitin-conjugating enzyme E2 K | UBE2K | P61086 | 0.44 | 0.24 | 0.65 | 2.86E−05 | 1.47E−04 |
| 6-phosphogluconolactonase | PGLS | O95336 | 0.44 | 0.26 | 0.63 | 3.16E−06 | 2.35E−05 |
| Protein SGT1 homolog | SUGT1 | Q9Y2Z0 | 0.44 | 0.25 | 0.62 | 5.79E−06 | 3.94E−05 |
| Thyroglobulin | TG | P01266 | 0.44 | 0.18 | 0.71 | 9.39E−04 | 2.93E−03 |
| Platelet-activating factor acetylhydrolase 2, | PAFAH2 | Q99487 | 0.44 | 0.13 | 0.76 | 5.15E−03 | 1.35E−02 |
| cytoplasmic | | | | | | | |
| Hypoxanthine-guanine phosphoribosyltransferase | HPRT1 | P00492 | 0.44 | 0.1 | 0.78 | 1.08E−02 | 2.59E−02 |
| Protein DJ-1 | PARK7 | Q99497 | 0.44 | 0.26 | 0.62 | 1.86E−06 | 1.51E−05 |
| Disks large homolog 2 | DLG2 | Q15700 | 0.44 | 0.26 | 0.62 | 2.01E−06 | 1.62E−05 |
| Alpha-L-iduronidase | IDUA | P35475 | 0.44 | 0.1 | 0.78 | 1.07E−02 | 2.56E−02 |
| Chymotrypsin-like elastase family member 1 | CELA1 | Q9UNI1 | 0.44 | 0.08 | 0.79 | 1.53E−02 | 3.53E−02 |
| Nuclear factor erythroid 2-related factor 1 | NFE2L1 | Q14494 | 0.43 | 0.21 | 0.65 | 1.14E−04 | 4.75E−04 |
| Non-homologous end-joining factor 1 | NHEJ1 | Q9H9Q4 | 0.43 | 0.2 | 0.66 | 2.92E−04 | 1.06E−03 |
| Glia maturation factor gamma | GMFG | O60234 | 0.43 | 0.25 | 0.61 | 1.95E−06 | 1.58E−05 |
| Protein HEXIM2 | HEXIM2 | Q96MH2 | 0.43 | 0.14 | 0.73 | 4.38E−03 | 1.16E−02 |
| Peptidyl-prolyl cis-trans isomerase NIMA- | PIN1 | Q13526 | 0.43 | 0.25 | 0.61 | 2.60E−06 | 2.01E−05 |
| interacting 1 | | | | | | | |
| Alpha-taxilin | TXLNA | P40222 | 0.43 | 0.18 | 0.68 | 6.20E−04 | 2.02E−03 |
| ATP-dependent 6-phosphofructokinase, muscle | PFKM | P08237 | 0.43 | 0.15 | 0.7 | 2.42E−03 | 6.82E−03 |
| type | | | | | | | |
| Glutamate--cysteine ligase regulatory subunit | GCLM | P48507 | 0.43 | 0.25 | 0.62 | 5.81E−06 | 3.94E−05 |
| Endophilin-B2 | SH3GLB2 | Q9NR46 | 0.43 | 0.2 | 0.66 | 1.98E−04 | 7.56E−04 |
| NADH-cytochrome b5 reductase 2 | CYB5R2 | Q6BCY4 | 0.43 | 0.09 | 0.77 | 1.34E−02 | 3.13E−02 |
| Transcription factor 4 | TCF4 | P15884 | 0.43 | 0.26 | 0.59 | 4.86E−07 | 4.95E−06 |
| Thioredoxin domain-containing protein 12 | TXNDC12 | O95881 | 0.43 | 0.26 | 0.6 | 5.65E−07 | 5.55E−06 |
| Serine protease HTRA2, mitochondrial | HTRA2 | O43464 | 0.43 | 0.15 | 0.7 | 2.42E−03 | 6.82E−03 |
| Cellular tumor antigen p53 | TP53 | P04637 | 0.43 | 0.07 | 0.8 | 2.07E−02 | 4.60E−02 |
| Bone morphogenetic protein 4 | BMP4 | P12644 | 0.43 | 0.1 | 0.76 | 1.04E−02 | 2.51E−02 |
| Secretoglobin family 1D member 2 | SCGB1D2 | O95969 | 0.43 | 0.1 | 0.77 | 1.20E−02 | 2.84E−02 |
| SLAM family member 8 | SLAMF8 | Q9P0V8 | 0.43 | 0.14 | 0.73 | 4.21E−03 | 1.12E−02 |
| C-X-C motif chemokine 9 | CXCL9 | Q07325 | 0.43 | 0.14 | 0.72 | 4.00E−03 | 1.07E−02 |
| Trem-like transcript 1 protein: Extracellular | TREML1 | Q86YW5 | 0.43 | 0.2 | 0.66 | 2.40E−04 | 8.93E−04 |
| domain, Ig-like V-type domain | | | | | | | |
| Zinc transporter 5 | SLC30A5 | Q8TAD4 | 0.43 | 0.22 | 0.64 | 5.62E−05 | 2.57E−04 |
| Peptidyl-prolyl cis-trans isomerase-like 1 | PPIL1 | Q9Y3C6 | 0.43 | 0.18 | 0.68 | 7.53E−04 | 2.40E−03 |
| Baculoviral IAP repeat-containing protein 2 | BIRC2 | Q13490 | 0.42 | 0.06 | 0.78 | 2.15E−02 | 4.75E−02 |
| E3 ubiquitin-protein ligase DTX3L | DTX3L | Q8TDB6 | 0.42 | 0.11 | 0.73 | 7.70E−03 | 1.92E−02 |
| Synaptosomal-associated protein 29 | SNAP29 | O95721 | 0.42 | 0.08 | 0.77 | 1.67E−02 | 3.82E−02 |
| Ig gamma-4, Kappa | IGHG4 | P01861 | 0.42 | 0.12 | 0.71 | 5.82E−03 | 1.50E−02 |
| ADP-ribosylation factor-binding protein GGA1 | GGA1 | Q9UJY5 | 0.42 | 0.26 | 0.58 | 2.52E−07 | 2.81E−06 |
| L-aminoadipate-semialdehyde dehydrogenase- | AASDHPPT | Q9NRN7 | 0.42 | 0.24 | 0.59 | 2.33E−06 | 1.85E−05 |
| phosphopantetheinyl transferase | | | | | | | |
| ADP-ribosylation factor-like protein 6 | ARL6 | Q9H0F7 | 0.42 | 0.1 | 0.73 | 9.73E−03 | 2.36E−02 |
| Macrosialin | CD68 | P34810 | 0.42 | 0.11 | 0.73 | 7.39E−03 | 1.85E−02 |
| Polymeric immunoglobulin receptor | PIGR | P01833 | 0.42 | 0.15 | 0.68 | 2.50E−03 | 7.00E−03 |
| Ubiquitin-fold modifier 1 | UFM1 | P61960 | 0.42 | 0.24 | 0.6 | 7.40E−06 | 4.84E−05 |
| Ribosome maturation protein SBDS | SBDS | Q9Y3A5 | 0.42 | 0.26 | 0.58 | 4.00E−07 | 4.21E−06 |
| Interstitial collagenase | MMP1 | P03956 | 0.42 | 0.21 | 0.64 | 1.05E−04 | 4.42E−04 |
| ATP synthase-coupling factor 6, mitochondrial | ATP5J | P18859 | 0.42 | 0.24 | 0.61 | 4.71E−06 | 3.31E−05 |
| Peroxisomal membrane protein PEX14: N-term | PEX14 | O75381 | 0.42 | 0.11 | 0.73 | 7.05E−03 | 1.78E−02 |
| Alpha-synuclein | SNCA | P37840 | 0.42 | 0.21 | 0.63 | 1.15E−04 | 4.76E−04 |
| Poly [ADP-ribose] polymerase 1: region 1 | PARP1 | P09874 | 0.42 | 0.12 | 0.72 | 6.48E−03 | 1.66E−02 |
| Tyrosine-protein kinase SYK: Protein kinase | SYK | P43405 | 0.41 | 0.18 | 0.64 | 5.41E−04 | 1.81E−03 |
| domain | | | | | | | |
| C-terminal-binding protein 1 | CTBP1 | Q13363 | 0.41 | 0.18 | 0.64 | 3.72E−04 | 1.30E−03 |
| Eukaryotic translation initiation factor 5A-2 | EIF5A2 | Q9GZV4 | 0.41 | 0.21 | 0.6 | 3.83E−05 | 1.87E−04 |
| F-actin-capping protein subunit alpha-1 | CAPZA1 | P52907 | 0.41 | 0.23 | 0.59 | 8.19E−06 | 5.25E−05 |
| Poly(rC)-binding protein 1 | PCBP1 | Q15365 | 0.41 | 0.22 | 0.6 | 3.36E−05 | 1.67E−04 |
| Autophagy protein 5 | ATG5 | Q9H1Y0 | 0.41 | 0.22 | 0.6 | 2.94E−05 | 1.50E−04 |
| Eukaryotic translation initiation factor 4B | EIF4B | P23588 | 0.41 | 0.22 | 0.59 | 2.00E−05 | 1.08E−04 |
| LDLR chaperone MESD | MESDC2 | Q14696 | 0.41 | 0.24 | 0.58 | 2.49E−06 | 1.93E−05 |
| Nucleoside diphosphate kinase, mitochondrial | NME4 | O00746 | 0.41 | 0.27 | 0.55 | 1.02E−08 | 1.53E−07 |
| Acetyl-CoA acetyltransferase, cytosolic | ACAT2 | Q9BWD1 | 0.41 | 0.26 | 0.55 | 5.31E−08 | 6.69E−07 |
| PEST proteolytic signal-containing nuclear | PCNP | Q8WW12 | 0.41 | 0.24 | 0.58 | 1.60E−06 | 1.32E−05 |
| protein | | | | | | | |
| Receptor-transporting protein 4 | RTP4 | Q96DX8 | 0.41 | 0.21 | 0.62 | 8.04E−05 | 3.51E−04 |
| Sorting nexin-5 | SNX5 | Q9Y5X3 | 0.41 | 0.21 | 0.6 | 4.47E−05 | 2.14E−04 |
| Nucleolysin TIAR | TIAL1 | Q01085 | 0.41 | 0.21 | 0.61 | 4.97E−05 | 2.32E−04 |
| Protein phosphatase inhibitor 2 | PPP1R2 | P41236 | 0.41 | 0.17 | 0.65 | 8.91E−04 | 2.79E−03 |
| Sorting nexin-3 | SNX3 | O60493 | 0.41 | 0.19 | 0.62 | 1.70E−04 | 6.64E−04 |
| NSFL1 cofactor p47 | NSFL1C | Q9UNZ2 | 0.41 | 0.2 | 0.61 | 9.69E−05 | 4.11E−04 |
| Endoplasmic reticulum aminopeptidase 1 | ERAP1 | Q9NZ08 | 0.41 | 0.18 | 0.63 | 3.55E−04 | 1.25E−03 |
| Moesin | MSN | P26038 | 0.41 | 0.08 | 0.73 | 1.33E−02 | 3.11E−02 |
| Histidine triad nucleotide-binding protein 2, | HINT2 | Q9BX68 | 0.41 | 0.17 | 0.66 | 7.30E−04 | 2.34E−03 |
| mitochondrial | | | | | | | |
| BolA-like protein 3 | BOLA3 | Q53S33 | 0.41 | 0.25 | 0.56 | 2.26E−07 | 2.53E−06 |
| Epididymal secretory protein E3-alpha | EDDM3A | Q14507 | 0.41 | 0.06 | 0.75 | 2.06E−02 | 4.58E−02 |
| Maspardin | SPG21 | Q9NZD8 | 0.4 | 0.18 | 0.62 | 3.75E−04 | 1.31E−03 |
| Dihydropteridine reductase | QDPR | P09417 | 0.4 | 0.17 | 0.63 | 7.46E−04 | 2.39E−03 |
| Ubiquitin-like protein 4A | UBL4A | P11441 | 0.4 | 0.2 | 0.59 | 6.09E−05 | 2.76E−04 |
| Transgelin-2 | TAGLN2 | P37802 | 0.4 | 0.21 | 0.6 | 5.15E−05 | 2.38E−04 |
| Peptidyl-prolyl cis-trans isomerase NIMA- | PIN4 | Q9Y237 | 0.4 | 0.24 | 0.56 | 1.83E−06 | 1.49E−05 |
| interacting 4 | | | | | | | |
| Tubulin--tyrosine ligase | TTL | Q8NG68 | 0.4 | 0.21 | 0.58 | 3.26E−05 | 1.63E−04 |
| SHC-transforming protein 1: Phosphotyrosine | SHC1 | P29353 | 0.4 | 0.22 | 0.58 | 1.22E−05 | 7.17E−05 |
| Interaction Domain | | | | | | | |
| 3′(2′),5′-bisphosphate nucleotidase 1 | BPNT1 | O95861 | 0.4 | 0.2 | 0.59 | 6.06E−05 | 2.74E−04 |
| N-alpha-acetyltransferase 10 | NAA10 | P41227 | 0.4 | 0.21 | 0.6 | 6.03E−05 | 2.74E−04 |
| Vacuolar protein-sorting-associated protein 25 | VPS25 | Q9BRG1 | 0.4 | 0.22 | 0.59 | 1.40E−05 | 8.07E−05 |
| Transcriptional activator protein Pur-beta | PURB | Q96QR8 | 0.4 | 0.24 | 0.57 | 1.29E−06 | 1.09E−05 |
| GMP reductase 2 | GMPR2 | Q9P2T1 | 0.4 | 0.18 | 0.63 | 5.23E−04 | 1.76E−03 |
| Hsp90alpha | HSP90AA1 | P07900 | 0.4 | 0.24 | 0.55 | 3.24E−07 | 3.52E−06 |
| Calcium/calmodulin-dependent protein kinase | CAMK2B | Q13554 | 0.4 | 0.2 | 0.6 | 8.11E−05 | 3.53E−04 |
| type II subunit beta | | | | | | | |
| Xaa-Pro aminopeptidase 1 | XPNPEP1 | Q9NQW7 | 0.4 | 0.22 | 0.59 | 2.18E−05 | 1.16E−04 |
| Tyrosine-protein kinase Fgr | FGR | P09769 | 0.4 | 0.19 | 0.62 | 2.69E−04 | 9.86E−04 |
| DnaJ homolog subfamily B member 1 | DNAJB1 | P25685 | 0.4 | 0.24 | 0.56 | 1.34E−06 | 1.14E−05 |
| Nascent polypeptide-associated complex subunit | NACA | Q13765 | 0.4 | 0.24 | 0.56 | 8.33E−07 | 7.61E−06 |
| alpha | | | | | | | |
| Fructose-bisphosphate aldolase A | ALDOA | P04075 | 0.4 | 0.22 | 0.58 | 8.80E−06 | 5.56E−05 |
| Choriogonadotropin subunit beta variant 2 | CGB2 | Q6NT52 | 0.4 | 0.19 | 0.62 | 2.48E−04 | 9.20E−04 |
| Transmembrane protein C1orf162 | C1orf162 | Q8NEQ5 | 0.4 | 0.18 | 0.63 | 5.24E−04 | 1.76E−03 |
| Dipeptidyl peptidase 2 | DPP7 | Q9UHL4 | 0.4 | 0.19 | 0.61 | 1.48E−04 | 5.86E−04 |
| Eukaryotic translation initiation factor 4E type 2 | EIF4E2 | O60573 | 0.4 | 0.12 | 0.68 | 5.64E−03 | 1.46E−02 |
| Ezrin | EZR | P15311 | 0.4 | 0.2 | 0.61 | 1.38E−04 | 5.54E−04 |
| Toll/interleukin-1 receptor domain-containing | TIRAP | P58753 | 0.4 | 0.22 | 0.59 | 1.25E−05 | 7.33E−05 |
| adapter protein | | | | | | | |
| Nuclear factor NF-kappa-B p105 subunit | NFKB1 | P19838 | 0.4 | 0.22 | 0.58 | 1.73E−05 | 9.62E−05 |
| Deoxyuridine 5′-triphosphate | DUT | P33316 | 0.4 | 0.25 | 0.55 | 2.81E−07 | 3.08E−06 |
| nucleotidohydrolase, mitochondrial | | | | | | | |
| Trem-like transcript 1 protein: immunoreceptor | TREML1 | Q86YW5 | 0.39 | 0.08 | 0.69 | 1.32E−02 | 3.10E−02 |
| tyrosine-based inhibition motif | | | | | | | |
| V-type immunoglobulin domain-containing | C10orf54 | Q9H7M9 | 0.39 | 0.21 | 0.57 | 3.04E−05 | 1.54E−04 |
| suppressor of T-cell activation: Cytoplasmic | | | | | | | |
| domain | | | | | | | |
| ELAV-like protein 1 | ELAVL1 | Q15717 | 0.39 | 0.06 | 0.71 | 1.92E−02 | 4.29E−02 |
| IST1 homolog | IST1 | P53990 | 0.39 | 0.2 | 0.58 | 6.69E−05 | 3.00E−04 |
| Peptidyl-prolyl cis-trans isomerase H | PPIH | O43447 | 0.39 | 0.21 | 0.58 | 4.12E−05 | 1.99E−04 |
| SUMO-activating enzyme subunit 2 | UBA2 | Q9UBT2 | 0.39 | 0.08 | 0.7 | 1.27E−02 | 2.99E−02 |
| 5′(3′)-deoxyribonucleotidase, cytosolic type | NT5C | Q8TCD5 | 0.39 | 0.11 | 0.66 | 5.50E−03 | 1.43E−02 |
| Dihydropyrimidinase-related protein 3 | DPYSL3 | Q14195 | 0.39 | 0.14 | 0.63 | 2.04E−03 | 5.82E−03 |
| Plastin-1 | PLS1 | Q14651 | 0.39 | 0.15 | 0.63 | 1.45E−03 | 4.29E−03 |
| 26S proteasome non-ATPase regulatory subunit | PSMD11 | O00231 | 0.39 | 0.19 | 0.6 | 1.75E−04 | 6.78E−04 |
| 11 | | | | | | | |
| Regulator of G-protein signaling 18 | RGS18 | Q9NS28 | 0.39 | 0.12 | 0.66 | 4.24E−03 | 1.13E−02 |
| Vimentin | VIM | P08670 | 0.39 | 0.14 | 0.64 | 2.19E−03 | 6.21E−03 |
| Glutaredoxin-3 | GLRX3 | O76003 | 0.39 | 0.21 | 0.57 | 1.68E−05 | 9.34E−05 |
| Docking protein 1 | DOK1 | Q99704 | 0.39 | 0.18 | 0.6 | 2.84E−04 | 1.03E−03 |
| Protein SCO2 homolog, mitochondrial | SCO2 | O43819 | 0.39 | 0.2 | 0.57 | 3.68E−05 | 1.81E−04 |
| Annexin A4 | ANXA4 | P09525 | 0.39 | 0.21 | 0.57 | 3.12E−05 | 1.57E−04 |
| GMP synthase [glutamine-hydrolyzing] | GMPS | P49915 | 0.39 | 0.21 | 0.57 | 2.29E−05 | 1.21E−04 |
| Inosine triphosphate pyrophosphatase | ITPA | Q9BY32 | 0.39 | 0.15 | 0.63 | 1.40E−03 | 4.16E−03 |
| Luteinizing hormone | CGA LHB | P01229\|P01215 | 0.39 | 0.2 | 0.58 | 3.82E−05 | 1.87E−04 |
| Protein 4.1 | EPB41 | P11171 | 0.39 | 0.19 | 0.6 | 2.03E−04 | 7.74E−04 |
| C-C motif chemokine 16 | CCL16 | O15467 | 0.39 | 0.07 | 0.71 | 1.70E−02 | 3.86E−02 |
| Adenylate kinase isoenzyme 1 | AK1 | P00568 | 0.39 | 0.16 | 0.62 | 9.92E−04 | 3.08E−03 |
| Casein kinase II 2-alpha′: 2-beta heterotetramer | CSNK2A2 | P67870\|P19784 | 0.39 | 0.2 | 0.58 | 7.00E−05 | 3.11E−04 |
| | CSNK2B | | | | | | |
| Serine/threonine-protein kinase WNK3 | WNK3 | Q9BYP7 | 0.39 | 0.14 | 0.65 | 2.83E−03 | 7.83E−03 |
| Vesicle-associated membrane protein-associated | VAPA | Q9P0L0 | 0.39 | 0.23 | 0.54 | 6.78E−07 | 6.44E−06 |
| protein A | | | | | | | |
| Alkaline phosphatase, placental type | ALPP | P05187 | 0.39 | 0.17 | 0.6 | 4.29E−04 | 1.48E−03 |
| Vesicle transport through interaction with t- | VTI1B | Q9UEU0 | 0.39 | 0.22 | 0.57 | 8.08E−06 | 5.20E−05 |
| SNAREs homolog 1B | | | | | | | |
| Beta-2-microglobulin | B2M | P61769 | 0.38 | 0.19 | 0.58 | 9.87E−05 | 4.18E−04 |
| Alcohol dehydrogenase class-3 | ADH5 | P11766 | 0.38 | 0.17 | 0.59 | 2.95E−04 | 1.06E−03 |
| Arf-GAP domain and FG repeat-containing | AGFG1 | P52594 | 0.38 | 0.22 | 0.53 | 1.86E−06 | 1.51E−05 |
| protein 1 | | | | | | | |
| Rho guanine nucleotide exchange factor 1 | ARHGEF1 | Q92888 | 0.38 | 0.14 | 0.61 | 1.73E−03 | 5.02E−03 |
| Splicing factor 45 | RBM17 | Q96I25 | 0.38 | 0.23 | 0.54 | 1.17E−06 | 1.03E−05 |
| Ubiquitin carboxyl-terminal hydrolase isozyme | UCHL5 | Q9Y5K5 | 0.38 | 0.15 | 0.6 | 9.32E−04 | 2.91E−03 |
| L5 | | | | | | | |
| Interferon-induced GTP-binding protein Mx1 | MX1 | P20591 | 0.38 | 0.11 | 0.64 | 5.52E−03 | 1.43E−02 |
| Esterase OVCA2 | OVCA2 | Q8WZ82 | 0.38 | 0.23 | 0.53 | 7.05E−07 | 6.59E−06 |
| Interleukin-1 receptor-associated kinase 4 | IRAK4 | Q9NWZ3 | 0.38 | 0.17 | 0.6 | 5.24E−04 | 1.76E−03 |
| Reticulon-4-interacting protein 1, mitochondrial | RTN4IP1 | Q8WWV3 | 0.38 | 0.21 | 0.56 | 1.96E−05 | 1.07E−04 |
| Ribose-phosphate pyrophosphokinase 2 | PRPS2 | P11908 | 0.38 | 0.08 | 0.69 | 1.37E−02 | 3.20E−02 |
| Phosphoribosyl pyrophosphate synthase- | PRPSAP2 | O60256 | 0.38 | 0.15 | 0.61 | 1.44E−03 | 4.24E−03 |
| associated protein 2 | | | | | | | |
| Eukaryotic translation initiation factor 2 subunit 1 | EIF2S1 | P05198 | 0.38 | 0.22 | 0.54 | 4.46E−06 | 3.16E−05 |
| Platelet glycoprotein VI | GP6 | Q9HCN6 | 0.38 | 0.22 | 0.53 | 4.22E−06 | 3.03E−05 |
| Granulocyte colony-stimulating factor | CSF3 | P09919 | 0.38 | 0.08 | 0.68 | 1.18E−02 | 2.80E−02 |
| Apoptosis-inducing factor 1, mitochondrial | AIFM1 | O95831 | 0.38 | 0.14 | 0.61 | 1.52E−03 | 4.46E−03 |
| Rho GDP-dissociation inhibitor 2 | ARHGDIB | P52566 | 0.38 | 0.23 | 0.54 | 1.55E−06 | 1.29E−05 |
| 3-keto-steroid reductase:Extracellular domain | HSD17B7 | P56937 | 0.38 | 0.19 | 0.57 | 7.73E−05 | 3.39E−04 |
| (partial) | | | | | | | |
| N-acetylserotonin O-methyltransferase-like | ASMTL | O95671 | 0.37 | 0.16 | 0.58 | 5.45E−04 | 1.82E−03 |
| protein | | | | | | | |
| E3 ubiquitin-protein ligase CBL | CBL | P22681 | 0.37 | 0.21 | 0.53 | 6.48E−06 | 4.33E−05 |
| Serine--tRNA ligase, mitochondrial | SARS2 | Q9NP81 | 0.37 | 0.2 | 0.53 | 8.92E−06 | 5.59E−05 |
| Gamma-aminobutyric acid receptor-associated | GABARAPL2 | P60520 | 0.37 | 0.2 | 0.54 | 2.06E−05 | 1.11E−04 |
| protein-like 2 | | | | | | | |
| 60S ribosome subunit biogenesis protein NIP7 | NIP7 | Q9Y221 | 0.37 | 0.18 | 0.55 | 1.22E−04 | 4.97E−04 |
| homolog | | | | | | | |
| Obg-like ATPase 1 | OLA1 | Q9NTK5 | 0.37 | 0.19 | 0.54 | 4.59E−05 | 2.18E−04 |
| Interferon-induced protein with tetratricopeptide | IFIT3 | O14879 | 0.37 | 0.08 | 0.66 | 1.37E−02 | 3.19E−02 |
| repeats 3 | | | | | | | |
| eIF-2-alpha kinase GCN2 | EIF2AK4 | Q9P2K8 | 0.37 | 0.12 | 0.62 | 3.32E−03 | 9.08E−03 |
| Protein S100-A5 | S100A5 | P33763 | 0.37 | 0.15 | 0.58 | 9.20E−04 | 2.87E−03 |
| GPN-loop GTPase 1 | GPN1 | Q9HCN4 | 0.37 | 0.16 | 0.58 | 6.58E−04 | 2.14E−03 |
| Ras-related protein Rab-21 | RAB21 | Q9UL25 | 0.37 | 0.21 | 0.53 | 9.85E−06 | 6.11E−05 |
| Gamma-soluble NSF attachment protein | NAPG | Q99747 | 0.37 | 0.21 | 0.53 | 7.13E−06 | 4.69E−05 |
| Annexin A7 | ANXA7 | P20073 | 0.37 | 0.19 | 0.55 | 4.05E−05 | 1.96E−04 |
| 3-oxoacyl-[acyl-carrier-protein] synthase, | OXSM | Q9NWU1 | 0.37 | 0.2 | 0.54 | 1.81E−05 | 9.98E−05 |
| mitochondrial | | | | | | | |
| Pyruvate dehydrogenase protein X component, | PDHX | O00330 | 0.37 | 0.22 | 0.52 | 8.82E−07 | 8.01E−06 |
| mitochondrial | | | | | | | |
| Radixin | RDX | P35241 | 0.37 | 0.19 | 0.55 | 4.12E−05 | 1.99E−04 |
| Transketolase | TKT | P29401 | 0.37 | 0.19 | 0.56 | 7.60E−05 | 3.34E−04 |
| Stress-induced-phosphoprotein 1 | STIP1 | P31948 | 0.37 | 0.22 | 0.53 | 2.97E−06 | 2.23E−05 |
| Cystatin-like 1 | CSTL1 | Q9H114 | 0.37 | 0.22 | 0.53 | 1.17E−06 | 1.02E−05 |
| 2′,3′-cyclic-nucleotide 3′-phosphodiesterase | CNP | P09543 | 0.37 | 0.19 | 0.55 | 7.55E−05 | 3.33E−04 |
| Alkaline phosphatase, placental-like | ALPPL2 | P10696 | 0.37 | 0.07 | 0.66 | 1.56E−02 | 3.57E−02 |
| KDEL motif-containing protein 2 | KDELC2 | Q7Z4H8 | 0.37 | 0.18 | 0.56 | 1.08E−04 | 4.51E−04 |
| Tropomyosin alpha-4 chain | TPM4 | P67936 | 0.37 | 0.17 | 0.57 | 2.89E−04 | 1.05E−03 |
| Signal transducer and activator of transcription 6 | STAT6 | P42226 | 0.36 | 0.16 | 0.56 | 4.73E−04 | 1.61E−03 |
| Chitinase-3-like protein 1 | CHI3L1 | P36222 | 0.36 | 0.22 | 0.49 | 4.24E−07 | 4.40E−06 |
| Galactokinase | GALK1 | P51570 | 0.36 | 0.17 | 0.55 | 2.39E−04 | 8.93E−04 |
| Vacuolar protein sorting-associated protein 4A | VPS4A | Q9UN37 | 0.36 | 0.17 | 0.54 | 1.63E−04 | 6.38E−04 |
| Homeobox protein DLX-4 | DLX4 | Q92988 | 0.36 | 0.2 | 0.53 | 9.96E−06 | 6.13E−05 |
| LanC-like protein 2 | LANCL2 | Q9NS86 | 0.36 | 0.12 | 0.59 | 2.80E−03 | 7.75E−03 |
| Enoyl-CoA delta isomerase 2, mitochondrial | ECI2 | O75521 | 0.36 | 0.21 | 0.51 | 4.35E−06 | 3.10E−05 |
| Protein S100-A6 | S100A6 | P06703 | 0.36 | 0.09 | 0.63 | 8.24E−03 | 2.04E−02 |
| Band 4.1-like protein 1 | EPB41L1 | Q9H4G0 | 0.36 | 0.1 | 0.62 | 6.74E−03 | 1.71E−02 |
| Delta-aminolevulinic acid dehydratase | ALAD | P13716 | 0.36 | 0.05 | 0.66 | 2.09E−02 | 4.62E−02 |
| Short/branched chain specific acyl-CoA | ACADSB | P45954 | 0.36 | 0.16 | 0.56 | 5.19E−04 | 1.75E−03 |
| dehydrogenase, mitochondrial | | | | | | | |
| Acyl-CoA synthetase family member 2, | ACSF2 | Q96CM8 | 0.36 | 0.18 | 0.54 | 1.08E−04 | 4.53E−04 |
| mitochondrial | | | | | | | |
| Lysine--tRNA ligase | KARS | Q15046 | 0.36 | 0.21 | 0.51 | 1.48E−06 | 1.25E−05 |
| Thymidylate kinase | DTYMK | P23919 | 0.36 | 0.18 | 0.54 | 7.96E−05 | 3.48E−04 |
| Protein kinase B gamma | AKT3 | Q9Y243 | 0.36 | 0.17 | 0.54 | 1.27E−04 | 5.17E−04 |
| Tyrosine-protein phosphatase non-receptor type 6 | PTPN6 | P29350 | 0.36 | 0.18 | 0.53 | 5.87E−05 | 2.68E−04 |
| 14-3-3 protein eta | YWHAH | Q04917 | 0.36 | 0.22 | 0.49 | 1.68E−07 | 1.94E−06 |
| Malectin | MLEC | Q14165 | 0.36 | 0.19 | 0.53 | 4.58E−05 | 2.17E−04 |
| Pregnancy zone protein | PZP | P20742 | 0.36 | 0.28 | 0.44 | 1.29E−16 | 6.97E−15 |
| UPF0160 protein MYG1, mitochondrial | C12orf10 | Q9HB07 | 0.36 | 0.15 | 0.56 | 5.58E−04 | 1.85E−03 |
| Glutathione peroxidase 7 | GPX7 | Q96SL4 | 0.36 | 0.15 | 0.57 | 8.38E−04 | 2.64E−03 |
| 5′-nucleotidase domain-containing protein 3 | NT5DC3 | Q86UY8 | 0.36 | 0.14 | 0.57 | 1.02E−03 | 3.16E−03 |
| Bis(5′-adenosyl)-triphosphatase | FHIT | P49789 | 0.36 | 0.19 | 0.54 | 5.27E−05 | 2.43E−04 |
| Filamin-A: Calponin Homology 2 | FLNA | P21333 | 0.35 | 0.18 | 0.53 | 8.83E−05 | 3.80E−04 |
| Adenylate kinase 2, mitochondrial | AK2 | P54819 | 0.35 | 0.19 | 0.5 | 8.48E−06 | 5.41E−05 |
| Protein-tyrosine kinase 2-beta: 4.1 protein, ezrin, | PTK2B | Q14289 | 0.35 | 0.16 | 0.53 | 2.99E−04 | 1.07E−03 |
| radixin, moesin domain | | | | | | | |
| Serine/threonine-protein phosphatase 2A 65 kDa | PPP2R1A | P30153 | 0.35 | 0.1 | 0.6 | 5.95E−03 | 1.53E−02 |
| regulatory subunit A alpha isoform | | | | | | | |
| Peroxisome proliferator-activated receptor alpha | PPARA | Q07869 | 0.35 | 0.05 | 0.65 | 2.26E−02 | 4.97E−02 |
| Importin subunit alpha-7 | KPNA6 | O60684 | 0.35 | 0.19 | 0.5 | 1.30E−05 | 7.56E−05 |
| Annexin A7 | ANXA7 | P20073 | 0.35 | 0.18 | 0.52 | 8.19E−05 | 3.56E−04 |
| Ski-like protein | SKIL | P12757 | 0.35 | 0.07 | 0.63 | 1.34E−02 | 3.13E−02 |
| Rho GTPase-activating protein 5 | ARHGAP5 | Q13017 | 0.35 | 0.19 | 0.51 | 1.16E−05 | 6.93E−05 |
| Protein HEXIM1 | HEXIM1 | O94992 | 0.35 | 0.21 | 0.49 | 1.21E−06 | 1.06E−05 |
| Interferon regulatory factor 3 | IRF3 | Q14653 | 0.35 | 0.2 | 0.5 | 3.57E−06 | 2.62E−05 |
| Succinate-semialdehyde dehydrogenase, | ALDH5A1 | P51649 | 0.35 | 0.07 | 0.63 | 1.41E−02 | 3.28E−02 |
| mitochondrial | | | | | | | |
| Neurogranin | NRGN | Q92686 | 0.35 | 0.15 | 0.54 | 5.22E−04 | 1.76E−03 |
| DNA-directed RNA polymerases I, II, and III | POLR2K | P53803 | 0.35 | 0.15 | 0.56 | 8.45E−04 | 2.66E−03 |
| subunit RPABC4 | | | | | | | |
| Acidic fibroblast growth factor intracellular- | FIBP | O43427 | 0.35 | 0.19 | 0.51 | 1.18E−05 | 6.99E−05 |
| binding protein | | | | | | | |
| Ras-related protein Rab-5B | RAB5B | P61020 | 0.35 | 0.18 | 0.52 | 8.19E−05 | 3.56E−04 |
| Mitogen-activated protein kinase 3 | MAPK3 | P27361 | 0.35 | 0.19 | 0.51 | 1.52E−05 | 8.66E−05 |
| Translationally-controlled tumor protein | TPT1 | P13693 | 0.35 | 0.22 | 0.49 | 2.82E−07 | 3.08E−06 |
| Casein kinase II 2-alpha:2-beta heterotetramer | CSNK2A1 | P67870\|P68400 | 0.35 | 0.2 | 0.5 | 3.97E−06 | 2.86E−05 |
| | CSNK2B | | | | | | |
| ATP-dependent RNA helicase DDX19B | DDX19B | Q9UMR2 | 0.35 | 0.17 | 0.53 | 1.19E−04 | 4.89E−04 |
| Appetite-regulating hormone | GHRL | Q9UBU3 | 0.35 | 0.21 | 0.49 | 7.77E−07 | 7.16E−06 |
| ATPase family AAA domain-containing protein 1 | ATAD1 | Q8NBU5 | 0.35 | 0.07 | 0.62 | 1.31E−02 | 3.07E−02 |
| Sorting nexin-8 | SNX8 | Q9Y5X2 | 0.35 | 0.19 | 0.5 | 1.16E−05 | 6.93E−05 |
| Cell differentiation protein RCD1 homolog | RQCD1 | Q92600 | 0.35 | 0.17 | 0.53 | 1.14E−04 | 4.72E−04 |
| Transgelin-2 | TAGLN2 | P37802 | 0.35 | 0.18 | 0.52 | 7.08E−05 | 3.13E−04 |
| Protein enabled homolog | ENAH | Q8N8S7 | 0.35 | 0.19 | 0.51 | 2.56E−05 | 1.33E−04 |
| Mothers against decapentaplegic homolog 3 | SMAD3 | P84022 | 0.34 | 0.16 | 0.52 | 1.84E−04 | 7.07E−04 |
| Serpin H1 | SERPINH1 | P50454 | 0.34 | 0.18 | 0.51 | 4.49E−05 | 2.14E−04 |
| Heat shock protein beta-1 | HSPB1 | P04792 | 0.34 | 0.2 | 0.48 | 2.99E−06 | 2.24E−05 |
| NEDD4-like E3 ubiquitin-protein ligase WWP1 | WWP1 | Q9H0M0 | 0.34 | 0.17 | 0.5 | 5.90E−05 | 2.69E−04 |
| Plexin domain-containing protein 2: Cytoplasmic | PLXDC2 | Q6UX71 | 0.34 | 0.16 | 0.53 | 2.88E−04 | 1.04E−03 |
| domain | | | | | | | |
| Tripartite motif-containing protein 3 | TRIM3 | O75382 | 0.34 | 0.14 | 0.54 | 1.08E−03 | 3.31E−03 |
| Delta(3,5)-Delta(2,4)-dienoyl-CoA isomerase, | ECH1 | Q13011 | 0.34 | 0.11 | 0.57 | 4.24E−03 | 1.13E−02 |
| mitochondrial | | | | | | | |
| Tropomodulin-3 | TMOD3 | Q9NYL9 | 0.34 | 0.15 | 0.52 | 3.60E−04 | 1.26E−03 |
| CCR4-NOT transcription complex subunit 1 | CNOT1 | A5YKK6 | 0.34 | 0.15 | 0.54 | 5.84E−04 | 1.92E−03 |
| Ras-related protein Rab-5C | RAB5C | P51148 | 0.34 | 0.16 | 0.53 | 2.88E−04 | 1.04E−03 |
| Histidine--tRNA ligase, cytoplasmic | HARS | P12081 | 0.34 | 0.19 | 0.49 | 9.11E−06 | 5.69E−05 |
| COMM domain-containing protein 9 | COMMD9 | Q9P000 | 0.34 | 0.19 | 0.49 | 5.77E−06 | 3.93E−05 |
| Tubulin-folding cofactor B | TBCB | Q99426 | 0.34 | 0.2 | 0.49 | 5.27E−06 | 3.66E−05 |
| Elongation factor 1-delta | EEF1D | P29692 | 0.34 | 0.19 | 0.49 | 1.34E−05 | 7.77E−05 |
| Phosphopantothenate--cysteine ligase | PPCS | Q9HAB8 | 0.34 | 0.19 | 0.5 | 1.25E−05 | 7.33E−05 |
| Developmentally-regulated GTP-binding protein | DRG1 | Q9Y295 | 0.34 | 0.19 | 0.48 | 3.88E−06 | 2.82E−05 |
| 1 | | | | | | | |
| ADP-ribosylation factor 4 | ARF4 | P18085 | 0.34 | 0.17 | 0.51 | 6.81E−05 | 3.04E−04 |
| Destrin | DSTN | P60981 | 0.34 | 0.07 | 0.6 | 1.30E−02 | 3.05E−02 |
| 28 kDa heat- and acid-stable phosphoprotein | PDAP1 | Q13442 | 0.34 | 0.21 | 0.47 | 3.81E−07 | 4.03E−06 |
| Glutaryl-CoA dehydrogenase, mitochondrial | GCDH | Q92947 | 0.34 | 0.17 | 0.52 | 1.38E−04 | 5.53E−04 |
| Thiosulfate sulfurtransferase/rhodanese-like | TSTD1 | Q8NFU3 | 0.34 | 0.14 | 0.53 | 8.15E−04 | 2.58E−03 |
| domain-containing protein 1 | | | | | | | |
| Ras-related protein Rab-3D | RAB3D | O95716 | 0.34 | 0.19 | 0.48 | 3.34E−06 | 2.46E−05 |
| Brain-derived neurotrophic factor | BDNF | P23560 | 0.34 | 0.05 | 0.62 | 2.08E−02 | 4.62E−02 |
| Tumor necrosis factor receptor superfamily | EDAR | Q9UNE0 | 0.34 | 0.11 | 0.58 | 4.40E−03 | 1.16E−02 |
| member EDAR | | | | | | | |
| Follicle stimulating hormone | CGA FSHB | P01225\|P01215 | 0.34 | 0.21 | 0.47 | 2.50E−07 | 2.79E−06 |
| Small glutamine-rich tetratricopeptide repeat- | SGTA | O43765 | 0.34 | 0.18 | 0.49 | 2.25E−05 | 1.19E−04 |
| containing protein alpha | | | | | | | |
| Glucose-6-phosphate isomerase | GPI | P06744 | 0.34 | 0.12 | 0.56 | 2.14E−03 | 6.07E−03 |
| Triosephosphate isomerase | TPI1 | P60174 | 0.34 | 0.17 | 0.5 | 4.34E−05 | 2.08E−04 |
| Peroxiredoxin-6 | PRDX6 | P30041 | 0.34 | 0.11 | 0.57 | 3.43E−03 | 9.32E−03 |
| Neurotensin/neuromedin N | NTS | P30990 | 0.34 | 0.17 | 0.5 | 6.29E−05 | 2.83E−04 |
| Methylmalonyl-CoA epimerase, mitochondrial | MCEE | Q96PE7 | 0.34 | 0.16 | 0.52 | 2.59E−04 | 9.55E−04 |
| Thymidine phosphorylase | TYMP | P19971 | 0.33 | 0.14 | 0.53 | 7.04E−04 | 2.27E−03 |
| Dynein light chain 2, cytoplasmic | DYNLL2 | Q96FJ2 | 0.33 | 0.19 | 0.47 | 3.01E−06 | 2.26E−05 |
| Stromal membrane-associated protein 1 | SMAP1 | Q8IYB5 | 0.33 | 0.19 | 0.47 | 6.50E−06 | 4.34E−05 |
| ADP-ribosylation factor-binding protein GGA3 | GGA3 | Q9NZ52 | 0.33 | 0.2 | 0.46 | 3.57E−07 | 3.84E−06 |
| Tyrosine-protein kinase JAK2 | JAK2 | O60674 | 0.33 | 0.19 | 0.48 | 4.93E−06 | 3.44E−05 |
| Smoothelin | SMTN | P53814 | 0.33 | 0.15 | 0.51 | 4.20E−04 | 1.44E−03 |
| Homer protein homolog 2 | HOMER2 | Q9NSB8 | 0.33 | 0.18 | 0.48 | 1.27E−05 | 7.42E−05 |
| Probable ATP-dependent RNA helicase DDX6 | DDX6 | P26196 | 0.33 | 0.19 | 0.46 | 2.17E−06 | 1.74E−05 |
| Ubiquitin carboxyl-terminal hydrolase 8 | USP8 | P40818 | 0.33 | 0.19 | 0.47 | 2.66E−06 | 2.05E−05 |
| Cytosol aminopeptidase | LAP3 | P28838 | 0.33 | 0.18 | 0.49 | 3.01E−05 | 1.52E−04 |
| Histone H2B type 2-E | HIST2H2BE | Q16778 | 0.33 | 0.15 | 0.52 | 3.49E−04 | 1.23E−03 |
| Chloride intracellular channel protein 4 | CLIC4 | Q9Y696 | 0.33 | 0.19 | 0.47 | 7.40E−06 | 4.84E−05 |
| Cytosol aminopeptidase | LAP3 | P28838 | 0.33 | 0.17 | 0.49 | 3.69E−05 | 1.81E−04 |
| Ras-related protein Rab-5A | RAB5A | P20339 | 0.33 | 0.17 | 0.49 | 5.22E−05 | 2.41E−04 |
| Annexin A11 | ANXA11 | P50995 | 0.33 | 0.19 | 0.48 | 7.93E−06 | 5.11E−05 |
| Alpha-endosulfine | ENSA | O43768 | 0.33 | 0.19 | 0.47 | 3.43E−06 | 2.53E−05 |
| Programmed cell death 6-interacting protein | PDCD6IP | Q8WUM4 | 0.33 | 0.16 | 0.51 | 1.47E−04 | 5.81E−04 |
| Calpain I | CAPN1 CAPNS1 | P04632\|P07384 | 0.33 | 0.16 | 0.5 | 1.53E−04 | 6.03E−04 |
| Cystatin-F | CST7 | O76096 | 0.33 | 0.1 | 0.56 | 4.81E−03 | 1.26E−02 |
| Vacuolar protein sorting-associated protein | VTA1 | Q9NP79 | 0.33 | 0.19 | 0.48 | 4.93E−06 | 3.44E−05 |
| VTA1 homolog | | | | | | | |
| GTP-binding nuclear protein Ran | RAN | P62826 | 0.33 | 0.17 | 0.49 | 3.78E−05 | 1.85E−04 |
| Mitogen-activated protein kinase 14 | MAPK14 | Q16539 | 0.33 | 0.16 | 0.5 | 1.41E−04 | 5.65E−04 |
| Pterin-4-alpha-carbinolamine dehydratase 2 | PCBD2 | Q9H0N5 | 0.33 | 0.07 | 0.59 | 1.45E−02 | 3.36E−02 |
| Uncharacterized protein C1orf198 | C1orf198 | Q9H425 | 0.33 | 0.1 | 0.55 | 4.62E−03 | 1.22E−02 |
| UPF0696 protein C11orf68 | C11orf68 | Q9H3H3 | 0.33 | 0.18 | 0.47 | 1.24E−05 | 7.29E−05 |
| TBC1 domain family member 5 | TBC1D5 | Q92609 | 0.33 | 0.13 | 0.54 | 1.12E−03 | 3.43E−03 |
| Protein max | MAX | P61244 | 0.32 | 0.19 | 0.45 | 2.94E−06 | 2.22E−05 |
| Tumor necrosis factor alpha-induced protein 8 | TNFAIP8 | O95379 | 0.32 | 0.13 | 0.52 | 9.64E−04 | 3.00E−03 |
| Fermitin family homolog 3 | FERMT3 | Q86UX7 | 0.32 | 0.13 | 0.52 | 1.27E−03 | 3.83E−03 |
| Ribulose-phosphate 3-epimerase | RPE | Q96AT9 | 0.32 | 0.12 | 0.51 | 1.31E−03 | 3.93E−03 |
| Aspartate--tRNA ligase, cytoplasmic | DARS | P14868 | 0.32 | 0.17 | 0.47 | 3.52E−05 | 1.74E−04 |
| Eukaryotic translation initiation factor 1b | EIF1B | O60739 | 0.32 | 0.18 | 0.46 | 4.84E−06 | 3.39E−05 |
| AP-1 complex subunit gamma-like 2 | AP1G2 | O75843 | 0.32 | 0.18 | 0.46 | 1.23E−05 | 7.25E−05 |
| Tumor susceptibility gene 101 protein | TSG101 | Q99816 | 0.32 | 0.18 | 0.46 | 1.16E−05 | 6.93E−05 |
| Fragile X mental retardation syndrome-related | FXR1 | P51114 | 0.32 | 0.11 | 0.52 | 2.34E−03 | 6.60E−03 |
| protein 1 | | | | | | | |
| Serine/threonine-protein phosphatase 2A 56 kDa | PPP2R5A | Q15172 | 0.32 | 0.15 | 0.49 | 1.63E−04 | 6.41E−04 |
| regulatory subunit alpha isoform | | | | | | | |
| Bifunctional polynucleotide phosphatase/kinase | PNKP | Q96T60 | 0.32 | 0.17 | 0.46 | 1.58E−05 | 8.96E−05 |
| 5-formyltetrahydrofolate cyclo-ligase | MTHFS | P49914 | 0.32 | 0.18 | 0.46 | 7.58E−06 | 4.93E−05 |
| Ras-related protein Ral-A | RALA | P11233 | 0.32 | 0.16 | 0.48 | 6.88E−05 | 3.07E−04 |
| RAC-alpha serine/threonine-protein kinase | AKT1 | P31749 | 0.32 | 0.17 | 0.46 | 1.98E−05 | 1.07E−04 |
| ATP-dependent RNA helicase DDX19A | DDX19A | Q9NUU7 | 0.32 | 0.15 | 0.48 | 1.33E−04 | 5.36E−04 |
| Acetyl-CoA acetyltransferase, mitochondrial | ACAT1 | P24752 | 0.32 | 0.16 | 0.47 | 4.79E−05 | 2.25E−04 |
| Glycogen synthase kinase-3 alpha | GSK3A | P49840 | 0.32 | 0.17 | 0.47 | 2.46E−05 | 1.28E−04 |
| Chloride intracellular channel protein 1 | CLIC1 | O00299 | 0.32 | 0.17 | 0.48 | 4.38E−05 | 2.10E−04 |
| Proto-oncogene vav | VAV1 | P15498 | 0.32 | 0.18 | 0.45 | 2.43E−06 | 1.90E−05 |
| 14-3-3 protein zeta/delta | YWHAZ | P63104 | 0.32 | 0.16 | 0.48 | 1.11E−04 | 4.62E−04 |
| Alpha-(1,6)-fucosyltransferase | FUT8 | Q9BYC5 | 0.32 | 0.05 | 0.59 | 2.24E−02 | 4.92E−02 |
| Acyl-CoA-binding domain-containing protein 6 | ACBD6 | Q9BR61 | 0.31 | 0.18 | 0.45 | 5.76E−06 | 3.93E−05 |
| Mothers against decapentaplegic homolog 2 | SMAD2 | Q15796 | 0.31 | 0.17 | 0.44 | 1.42E−05 | 8.18E−05 |
| Alpha-enolase | ENO1 | P06733 | 0.31 | 0.15 | 0.48 | 2.21E−04 | 8.32E−04 |
| 39S ribosomal protein L55, mitochondrial | MRPL55 | Q7Z7F7 | 0.31 | 0.15 | 0.46 | 9.08E−05 | 3.89E−04 |
| Ras-related protein Rab-22A | RAB22A | Q9UL26 | 0.31 | 0.12 | 0.5 | 1.66E−03 | 4.85E−03 |
| Calcyclin-binding protein | CACYBP | Q9HB71 | 0.31 | 0.19 | 0.44 | 1.11E−06 | 9.82E−06 |
| Nuclear protein localization protein 4 homolog | NPLOC4 | Q8TAT6 | 0.31 | 0.17 | 0.44 | 1.06E−05 | 6.45E−05 |
| Glutathione S-transferase kappa 1 | GSTK1 | Q9Y2Q3 | 0.31 | 0.17 | 0.44 | 7.46E−06 | 4.86E−05 |
| Arfaptin-1 | ARFIP1 | P53367 | 0.31 | 0.16 | 0.47 | 5.88E−05 | 2.68E−04 |
| Ras-related protein Rab-31 | RAB31 | Q13636 | 0.31 | 0.18 | 0.45 | 6.86E−06 | 4.55E−05 |
| Peroxisomal carnitine O-octanoyltransferase | CROT | Q9UKG9 | 0.31 | 0.12 | 0.5 | 1.60E−03 | 4.69E−03 |
| Quinone oxidoreductase | CRYZ | Q08257 | 0.31 | 0.09 | 0.54 | 5.41E−03 | 1.41E−02 |
| SCP2 sterol-binding domain-containing protein 1 | SCP2D1 | Q9UJQ7 | 0.31 | 0.08 | 0.53 | 7.50E−03 | 1.87E−02 |
| Phosphoglycerate mutase 2 | PGAM2 | P15259 | 0.31 | 0.11 | 0.51 | 2.69E−03 | 7.47E−03 |
| Malate dehydrogenase, mitochondrial | MDH2 | P40926 | 0.31 | 0.16 | 0.46 | 4.39E−05 | 2.10E−04 |
| CD320 antigen | CD320 | Q9NPF0 | 0.31 | 0.05 | 0.57 | 1.82E−02 | 4.11E−02 |
| SH3 domain-binding glutamic acid-rich-like | SH3BGRL3 | Q9H299 | 0.31 | 0.17 | 0.46 | 2.07E−05 | 1.11E−04 |
| protein 3 | | | | | | | |
| UDP-glucose 6-dehydrogenase | UGDH | O60701 | 0.31 | 0.12 | 0.5 | 1.15E−03 | 3.49E−03 |
| Snurportin-1 | SNUPN | O95149 | 0.31 | 0.17 | 0.45 | 9.93E−06 | 6.12E−05 |
| Threonine--tRNA ligase, cytoplasmic | TARS | P26639 | 0.31 | 0.18 | 0.45 | 4.94E−06 | 3.44E−05 |
| Cytochrome c oxidase subunit 5A, mitochondrial | COX5A | P20674 | 0.31 | 0.19 | 0.43 | 3.28E−07 | 3.55E−06 |
| T-complex protein 1 subunit alpha | TCP1 | P17987 | 0.31 | 0.12 | 0.5 | 1.34E−03 | 4.00E−03 |
| Branched-chain-amino-acid aminotransferase, | BCAT2 | O15382 | 0.31 | 0.15 | 0.47 | 1.50E−04 | 5.93E−04 |
| mitochondrial | | | | | | | |
| Ribosomal protein S6 kinase alpha-3 | RPS6KA3 | P51812 | 0.31 | 0.15 | 0.47 | 1.43E−04 | 5.70E−04 |
| Dynein light chain roadblock-type 1 | DYNLRB1 | Q9NP97 | 0.31 | 0.17 | 0.44 | 1.41E−05 | 8.12E−05 |
| Annexin A11 | ANXA1 | P04083 | 0.31 | 0.09 | 0.53 | 5.45E−03 | 1.42E−02 |
| Growth factor receptor-bound protein 2 | GRB2 | P62993 | 0.31 | 0.17 | 0.45 | 1.09E−05 | 6.58E−05 |
| Thioredoxin-dependent peroxide reductase, | PRDX3 | P30048 | 0.31 | 0.17 | 0.45 | 1.41E−05 | 8.12E−05 |
| mitochondrial | | | | | | | |
| Fatty acid synthase | FASN | P49327 | 0.31 | 0.15 | 0.46 | 8.65E−05 | 3.74E−04 |
| Transforming protein RhoA | RHOA | P61586 | 0.31 | 0.17 | 0.46 | 2.53E−05 | 1.32E−04 |
| E3 ubiquitin-protein ligase CHIP | STUB1 | Q9UNE7 | 0.3 | 0.13 | 0.48 | 6.71E−04 | 2.17E−03 |
| Transducin beta-like protein 2 | TBL2 | Q9Y4P3 | 0.3 | 0.17 | 0.43 | 4.89E−06 | 3.42E−05 |
| ADP-ribosylation factor GTPase-activating | ARFGAP2 | Q8N6H7 | 0.3 | 0.18 | 0.42 | 1.76E−06 | 1.44E−05 |
| protein 2 | | | | | | | |
| Alanine--tRNA ligase, cytoplasmic | AARS | P49588 | 0.3 | 0.15 | 0.46 | 1.45E−04 | 5.76E−04 |
| Haloacid dehalogenase-like hydrolase domain- | HDHD2 | Q9HOR4 | 0.3 | 0.14 | 0.45 | 1.41E−04 | 5.65E−04 |
| containing protein 2 | | | | | | | |
| Nucleosome assembly protein 1-like 1 | NAP1L1 | P55209 | 0.3 | 0.15 | 0.46 | 1.32E−04 | 5.32E−04 |
| Bifunctional 3′-phosphoadenosine 5′- | PAPSS1 | O43252 | 0.3 | 0.08 | 0.53 | 8.38E−03 | 2.07E−02 |
| phosphosulfate synthase 1 | | | | | | | |
| Histone H2A type 3 | HIST3H2A | Q7L7L0 | 0.3 | 0.12 | 0.49 | 1.18E−03 | 3.57E−03 |
| Folate receptor gamma | FOLR3 | P41439 | 0.3 | 0.09 | 0.51 | 5.03E−03 | 1.32E−02 |
| Casein kinase I isoform gamma-2 | CSNK1G2 | P78368 | 0.3 | 0.16 | 0.44 | 2.68E−05 | 1.39E−04 |
| Deoxycytidylate deaminase | DCTD | P32321 | 0.3 | 0.16 | 0.43 | 1.43E−05 | 8.19E−05 |
| Calcium-binding protein 39 | CAB39 | Q9Y376 | 0.3 | 0.17 | 0.43 | 5.86E−06 | 3.97E−05 |
| Cob(I)yrinic acid a,c-diamide | MMAB | Q96EY8 | 0.3 | 0.14 | 0.45 | 2.47E−04 | 9.18E−04 |
| adenosyltransferase, mitochondrial | | | | | | | |
| 5′(3′)-deoxyribonucleotidase, mitochondrial | NT5M | Q9NPB1 | 0.3 | 0.17 | 0.43 | 7.02E−06 | 4.63E−05 |
| Alcohol dehydrogenase 6 | ADH6 | P28332 | 0.3 | 0.15 | 0.45 | 8.24E−05 | 3.58E−04 |
| Nuclear apoptosis-inducing factor 1 | NAIF1 | Q69YI7 | 0.3 | 0.09 | 0.51 | 4.71E−03 | 1.24E−02 |
| Ras-related protein Ral-B | RALB | P11234 | 0.3 | 0.15 | 0.45 | 1.21E−04 | 4.97E−04 |
| Glutathione S-transferase Mu 4 | GSTM4 | Q03013 | 0.3 | 0.16 | 0.44 | 2.18E−05 | 1.16E−04 |
| AN1-type zinc finger protein 1 | ZFAND1 | Q8TCF1 | 0.3 | 0.15 | 0.45 | 6.86E−05 | 3.06E−04 |
| Homer protein homolog 1 | HOMER1 | Q86YM7 | 0.3 | 0.18 | 0.42 | 1.67E−06 | 1.38E−05 |
| Protein kinase C theta type | PRKCQ | Q04759 | 0.3 | 0.14 | 0.45 | 1.70E−04 | 6.64E−04 |
| Glyceraldehyde-3-phosphate dehydrogenase | GAPDH | P04406 | 0.3 | 0.17 | 0.42 | 3.22E−06 | 2.40E−05 |
| Cofilin-1 | CFL1 | P23528 | 0.3 | 0.08 | 0.53 | 7.40E−03 | 1.85E−02 |
| Alpha-soluble NSF attachment protein | NAPA | P54920 | 0.3 | 0.16 | 0.44 | 2.88E−05 | 1.47E−04 |
| Protein disulfide-isomerase A3 | PDIA3 | P30101 | 0.3 | 0.13 | 0.47 | 4.08E−04 | 1.41E−03 |
| Calcium uptake protein 2, mitochondrial | EFHA1 | Q8IYU8 | 0.3 | 0.15 | 0.44 | 4.99E−05 | 2.33E−04 |
| Translational activator of cytochrome c oxidase 1 | TACO1 | Q9BSH4 | 0.3 | 0.18 | 0.43 | 2.81E−06 | 2.14E−05 |
| Vasodilator-stimulated phosphoprotein | VASP | P50552 | 0.3 | 0.15 | 0.45 | 7.27E−05 | 3.21E−04 |
| Crk-like protein | CRKL | P46109 | 0.3 | 0.16 | 0.44 | 2.42E−05 | 1.27E−04 |
| UBX domain-containing protein 4: Cytoplasmic | UBXN4 | Q92575 | 0.3 | 0.07 | 0.53 | 1.02E−02 | 2.46E−02 |
| domain 2 | | | | | | | |
| UBX domain-containing protein 4: Cytoplasmic | UBXN4 | Q92575 | 0.3 | 0.15 | 0.45 | 9.09E−05 | 3.89E−04 |
| domain 1 | | | | | | | |
| Signal transducer and activator of transcription 3 | STAT3 | P40763 | 0.29 | 0.14 | 0.44 | 1.16E−04 | 4.77E−04 |
| Tubulin-specific chaperone E | TBCE | Q15813 | 0.29 | 0.17 | 0.41 | 4.51E−06 | 3.19E−05 |
| Regulator of G-protein signaling 10 | RGS10 | O43665 | 0.29 | 0.14 | 0.44 | 1.41E−04 | 5.65E−04 |
| Phosphomevalonate kinase | PMVK | Q15126 | 0.29 | 0.11 | 0.47 | 1.37E−03 | 4.07E−03 |
| Serine/threonine-protein kinase 24 | STK24 | Q9Y6E0 | 0.29 | 0.16 | 0.42 | 1.65E−05 | 9.22E−05 |
| Beta-defensin 4A | DEFB4A | O15263 | 0.29 | 0.14 | 0.43 | 1.23E−04 | 5.02E−04 |
| DNA dC−>dU-editing enzyme APOBEC-3G | APOBEC3G | Q9HC16 | 0.29 | 0.15 | 0.44 | 1.09E−04 | 4.55E−04 |
| Glucosamine 6-phosphate N-acetyltransferase | GNPNAT1 | Q96EK6 | 0.29 | 0.16 | 0.43 | 2.36E−05 | 1.24E−04 |
| LanC-like protein 1 | LANCL1 | O43813 | 0.29 | 0.06 | 0.51 | 1.21E−02 | 2.86E−02 |
| Purine nucleoside phosphorylase | PNP | P00491 | 0.29 | 0.13 | 0.45 | 4.98E−04 | 1.69E−03 |
| cGMP-specific 3′,5′-cyclic phosphodiesterase | PDE5A | O76074 | 0.29 | 0.16 | 0.43 | 1.61E−05 | 9.09E−05 |
| Microtubule-associated protein RP/EB family | MAPRE3 | Q9UPY8 | 0.29 | 0.12 | 0.45 | 6.47E−04 | 2.11E−03 |
| member 3 | | | | | | | |
| Ubiquitin recognition factor in ER-associated | UFD1L | Q92890 | 0.29 | 0.17 | 0.41 | 4.38E−06 | 3.12E−05 |
| degradation protein 1 | | | | | | | |
| Ras-related protein Rab-11A | RAB11A | P62491 | 0.29 | 0.14 | 0.44 | 1.42E−04 | 5.66E−04 |
| Rho-related GTP-binding protein RhoC | RHOC | P08134 | 0.29 | 0.14 | 0.44 | 1.46E−04 | 5.80E−04 |
| Omega-amidase NIT2 | NIT2 | Q9NQR4 | 0.29 | 0.13 | 0.45 | 2.96E−04 | 1.07E−03 |
| Phosphatidylinositol 5-phosphate 4-kinase type-2 | PIP4K2B | P78356 | 0.29 | 0.13 | 0.46 | 3.37E−04 | 1.19E−03 |
| beta | | | | | | | |
| Histone H2B type 3-B | HIST3H2BB | Q8N257 | 0.29 | 0.14 | 0.45 | 1.66E−04 | 6.50E−04 |
| Ras-related C3 botulinum toxin substrate 2 | RAC2 | P15153 | 0.29 | 0.15 | 0.43 | 3.80E−05 | 1.86E−04 |
| Isocitrate dehydrogenase [NAD] subunit gamma, | IDH3G | P51553 | 0.29 | 0.11 | 0.46 | 1.28E−03 | 3.85E−03 |
| mitochondrial | | | | | | | |
| Tyrosine-protein kinase CSK | CSK | P41240 | 0.29 | 0.14 | 0.44 | 1.43E−04 | 5.70E−04 |
| Nucleoside diphosphate kinase B | NME2 | P22392 | 0.29 | 0.15 | 0.43 | 3.38E−05 | 1.68E−04 |
| Calcineurin | PPP3CA | P63098\|Q08209 | 0.29 | 0.13 | 0.44 | 3.92E−04 | 1.36E−03 |
| | PPP3R1 | | | | | | |
| AMP Kinase (alpha2beta2gamma1) | PRKAA2 | P54619\|O43741\|P54646 | 0.29 | 0.15 | 0.42 | 5.39E−05 | 2.48E−04 |
| | PRKAB2 | | | | | | |
| | PRKAG1 | | | | | | |
| Protein disulfide-isomerase A6 | PDIA6 | Q15084 | 0.29 | 0.16 | 0.41 | 4.60E−06 | 3.25E−05 |
| Serine-rich single-pass membrane protein 1 | SSMEM1 | Q8WWF3 | 0.29 | 0.13 | 0.44 | 2.69E−04 | 9.86E−04 |
| FAS-associated factor 2 | FAF2 | Q96CS3 | 0.29 | 0.16 | 0.43 | 2.26E−05 | 1.20E−04 |
| Interferon regulatory factor 6 | IRF6 | O14896 | 0.29 | 0.09 | 0.48 | 3.91E−03 | 1.05E−02 |
| 1-phosphatidylinositol 4,5-bisphosphate | PLCB1 | Q9NQ66 | 0.28 | 0.05 | 0.5 | 1.60E−02 | 3.67E−02 |
| phosphodiesterase beta-1 | | | | | | | |
| Actin-related protein 2/3 complex subunit 1B | ARPC1B | O15143 | 0.28 | 0.15 | 0.4 | 1.38E−05 | 7.98E−05 |
| Leukotriene B4 receptor 1 | LTB4R | Q15722 | 0.28 | 0.13 | 0.43 | 3.13E−04 | 1.12E−03 |
| Serine/threonine-protein kinase PAK 4 | PAK4 | O96013 | 0.28 | 0.13 | 0.43 | 2.09E−04 | 7.93E−04 |
| Thioredoxin-like protein 4B | TXNL4B | Q9NX01 | 0.28 | 0.17 | 0.39 | 6.33E−07 | 6.14E−06 |
| Tyrosine-protein kinase Tec | TEC | P42680 | 0.28 | 0.07 | 0.49 | 1.01E−02 | 2.44E−02 |
| 10 kDa heat shock protein, mitochondrial | HSPE1 | P61604 | 0.28 | 0.15 | 0.4 | 2.55E−05 | 1.33E−04 |
| Mycophenolic acid acyl-glucuronide esterase, | ABHD10 | Q9NUJ1 | 0.28 | 0.15 | 0.42 | 4.77E−05 | 2.25E−04 |
| mitochondrial | | | | | | | |
| Charged multivesicular body protein 2a | CHMP2A | O43633 | 0.28 | 0.16 | 0.41 | 8.76E−06 | 5.54E−05 |
| Acylpyruvase FAHD1, mitochondrial | FAHD1 | Q6P587 | 0.28 | 0.15 | 0.42 | 4.50E−05 | 2.14E−04 |
| Glycine amidinotransferase, mitochondrial | GATM | P50440 | 0.28 | 0.13 | 0.43 | 2.99E−04 | 1.07E−03 |
| Isocitrate dehydrogenase [NADP] cytoplasmic | IDH1 | O75874 | 0.28 | 0.11 | 0.46 | 1.27E−03 | 3.82E−03 |
| Protein phosphatase 1 regulatory subunit 14A | PPP1R14A | Q96A00 | 0.28 | 0.16 | 0.39 | 3.30E−06 | 2.44E−05 |
| beta-adrenergic receptor kinase 1 | ADRBK1 | P25098 | 0.28 | 0.13 | 0.43 | 1.64E−04 | 6.42E−04 |
| AH receptor-interacting protein | AIP | O00170 | 0.28 | 0.13 | 0.43 | 2.22E−04 | 8.34E−04 |
| Sorting nexin-4 | SNX4 | O95219 | 0.28 | 0.15 | 0.41 | 1.97E−05 | 1.07E−04 |
| C-type lectin domain family 1 member B | CLEC1B | Q9P126 | 0.28 | 0.12 | 0.45 | 8.67E−04 | 2.73E−03 |
| Uncharacterized protein C5orf46 | C5orf46 | Q6UWT4 | 0.28 | 0.11 | 0.45 | 1.36E−03 | 4.05E−03 |
| ATP synthase subunit O, mitochondrial | ATP50 | P48047 | 0.28 | 0.16 | 0.4 | 4.42E−06 | 3.14E−05 |
| PRKC apoptosis WT1 regulator protein | PAWR | Q96IZ0 | 0.28 | 0.12 | 0.44 | 4.38E−04 | 1.50E−03 |
| Mothers against decapentaplegic homolog 1 | SMAD1 | Q15797 | 0.28 | 0.11 | 0.45 | 1.32E−03 | 3.97E−03 |
| DnaJ homolog subfamily C member 16 | DNAJC16 | Q9Y2G8 | 0.27 | 0.13 | 0.4 | 1.44E−04 | 5.72E−04 |
| EH domain-containing protein 4 | EHD4 | Q9H223 | 0.27 | 0.13 | 0.42 | 2.40E−04 | 8.95E−04 |
| PDZ and LIM domain protein 1 | PDLIM1 | O00151 | 0.27 | 0.14 | 0.4 | 3.20E−05 | 1.61E−04 |
| 3-hydroxyisobutyryl-CoA hydrolase, | HIBCH | Q6NVY1 | 0.27 | 0.15 | 0.39 | 7.99E−06 | 5.14E−05 |
| mitochondrial | | | | | | | |
| Endophilin-A1 | SH3GL2 | Q99962 | 0.27 | 0.15 | 0.39 | 1.08E−05 | 6.54E−05 |
| T-complex protein 1 subunit epsilon | CCT5 | P48643 | 0.27 | 0.12 | 0.43 | 5.48E−04 | 1.82E−03 |
| Phosphatidylinositol 5-phosphate 4-kinase type-2 | PIP4K2A | P48426 | 0.27 | 0.12 | 0.41 | 2.80E−04 | 1.02E−03 |
| alpha | | | | | | | |
| T-complex protein 1 subunit theta | CCT8 | P50990 | 0.27 | 0.12 | 0.43 | 5.43E−04 | 1.81E−03 |
| Protein FEV | FEV | Q99581 | 0.27 | 0.14 | 0.39 | 2.30E−05 | 1.22E−04 |
| Stress-70 protein, mitochondrial | HSPA9 | P38646 | 0.27 | 0.04 | 0.5 | 2.05E−02 | 4.57E−02 |
| Cytoplasmic protein NCK2 | NCK2 | O43639 | 0.27 | 0.16 | 0.39 | 5.50E−06 | 3.80E−05 |
| Rho-associated protein kinase 2 | ROCK2 | O75116 | 0.27 | 0.14 | 0.4 | 4.81E−05 | 2.26E−04 |
| Toll-interacting protein | TOLLIP | Q9H0E2 | 0.27 | 0.13 | 0.4 | 7.58E−05 | 3.34E−04 |
| Liprin-alpha-1 | PPFIA1 | Q13136 | 0.27 | 0.11 | 0.42 | 7.20E−04 | 2.31E−03 |
| TRAF family member-associated NF-kappa-B | TANK | Q92844 | 0.27 | 0.06 | 0.47 | 9.82E−03 | 2.37E−02 |
| activator | | | | | | | |
| 14-3-3 protein beta/alpha | YWHAB | P31946 | 0.27 | 0.15 | 0.4 | 1.89E−05 | 1.04E−04 |
| Docking protein 2 | DOK2 | O60496 | 0.27 | 0.14 | 0.4 | 5.07E−05 | 2.36E−04 |
| Protein argonaute-1 | AGO1 | Q9UL18 | 0.27 | 0.11 | 0.42 | 7.74E−04 | 2.46E−03 |
| Protein canopy homolog 4 | CNPY4 | Q8N129 | 0.27 | 0.12 | 0.43 | 6.58E−04 | 2.14E−03 |
| Ribosomal protein S6 kinase beta-1 | RPS6KB1 | P23443 | 0.27 | 0.1 | 0.45 | 2.48E−03 | 6.96E−03 |
| Bis(5′-nucleosyl)-tetraphosphatase | NUDT2 | P50583 | 0.27 | 0.14 | 0.41 | 6.78E−05 | 3.03E−04 |
| [asymmetrical] | | | | | | | |
| Charged multivesicular body protein 2b | CHMP2B | Q9UQN3 | 0.27 | 0.14 | 0.4 | 6.01E−05 | 2.73E−04 |
| Phosphomannomutase 1 | PMM1 | Q92871 | 0.27 | 0.04 | 0.5 | 2.19E−02 | 4.83E−02 |
| Citrate synthase, mitochondrial | CS | O75390 | 0.27 | 0.15 | 0.39 | 1.15E−05 | 6.91E−05 |
| D-tyrosyl-tRNA(Tyr) deacylase 1 | DTD1 | Q8TEA8 | 0.27 | 0.14 | 0.39 | 2.99E−05 | 1.52E−04 |
| C-Myc-binding protein | MYCBP | Q99417 | 0.27 | 0.14 | 0.39 | 1.89E−05 | 1.04E−04 |
| Myosin regulatory light chain 12A | MYL12A | P19105 | 0.27 | 0.15 | 0.4 | 2.11E−05 | 1.13E−04 |
| Docking protein 2 | DOK2 | O60496 | 0.27 | 0.15 | 0.39 | 1.18E−05 | 7.02E−05 |
| Tyrosine-protein kinase Lyn, isoform B | LYN | P07948 | 0.27 | 0.14 | 0.4 | 3.54E−05 | 1.75E−04 |
| MAP kinase-activated protein kinase 2 | MAPKAPK2 | P49137 | 0.27 | 0.13 | 0.41 | 1.53E−04 | 6.04E−04 |
| Aflatoxin B1 aldehyde reductase member 2 | AKR7A2 | O43488 | 0.27 | 0.13 | 0.41 | 2.01E−04 | 7.69E−04 |
| 3-hydroxyacyl-CoA dehydrogenase type-2 | HSD17B10 | Q99714 | 0.27 | 0.16 | 0.38 | 1.27E−06 | 1.09E−05 |
| 3-phosphoinositide-dependent protein kinase 1 | PDPK1 | O15530 | 0.27 | 0.13 | 0.42 | 2.86E−04 | 1.04E−03 |
| Dual 3′,5′-cyclic-AMP and -GMP | PDE11A | Q9HCR9 | 0.27 | 0.09 | 0.46 | 3.59E−03 | 9.71E−03 |
| phosphodiesterase 11A | | | | | | | |
| Copine-1: Ca2+-dependent membrane-targeting | CPNE1 | Q99829 | 0.27 | 0.13 | 0.4 | 1.15E−04 | 4.76E−04 |
| module domains 1 and 2 | | | | | | | |
| RAC-beta serine/threonine-protein kinase | AKT2 | P31751 | 0.27 | 0.15 | 0.39 | 1.69E−05 | 9.39E−05 |
| Eukaryotic translation initiation factor 4H | EIF4H | Q15056 | 0.27 | 0.14 | 0.39 | 3.04E−05 | 1.54E−04 |
| Stromal interaction molecule 1: Cytoplasmic | STIM1 | Q13586 | 0.27 | 0.11 | 0.44 | 1.14E−03 | 3.47E−03 |
| domain | | | | | | | |
| Dynamin-2 | DNM2 | P50570 | 0.26 | 0.12 | 0.41 | 2.64E−04 | 9.72E−04 |
| Alpha- and gamma-adaptin-binding protein p34 | AAGAB | Q6PD74 | 0.26 | 0.06 | 0.46 | 1.01E−02 | 2.43E−02 |
| Cyclin-dependent kinase 4 inhibitor D | CDKN2D | P55273 | 0.26 | 0.12 | 0.41 | 3.61E−04 | 1.26E−03 |
| Microtubule-associated protein RP/EB family | MAPRE1 | Q15691 | 0.26 | 0.14 | 0.37 | 8.87E−06 | 5.58E−05 |
| member 1 | | | | | | | |
| CD177 antigen | CD177 | Q8N6Q3 | 0.26 | 0.06 | 0.46 | 9.34E−03 | 2.27E−02 |
| Fumarate hydratase, mitochondrial | FH | P07954 | 0.26 | 0.1 | 0.43 | 1.22E−03 | 3.70E−03 |
| Derlin-1 | DERL1 | Q9BUN8 | 0.26 | 0.07 | 0.46 | 6.94E−03 | 1.75E−02 |
| Zyxin | ZYX | Q15942 | 0.26 | 0.09 | 0.43 | 2.79E−03 | 7.74E−03 |
| Hepatocyte growth factor-regulated tyrosine | HGS | O14964 | 0.26 | 0.12 | 0.41 | 4.84E−04 | 1.64E−03 |
| kinase substrate | | | | | | | |
| Endoplasmic reticulum resident protein 29 | ERP29 | P30040 | 0.26 | 0.14 | 0.37 | 1.82E−05 | 1.01E−04 |
| Arf-GAP with GTPase, ANK repeat and PH | AGAP2 | Q99490 | 0.26 | 0.12 | 0.41 | 3.49E−04 | 1.23E−03 |
| domain-containing protein 2 | | | | | | | |
| Acyl-coenzyme A thioesterase 8 | ACOT8 | O14734 | 0.26 | 0.07 | 0.45 | 7.44E−03 | 1.86E−02 |
| Myosin regulatory light chain 12B | MYL12B | O14950 | 0.26 | 0.12 | 0.4 | 2.30E−04 | 8.63E−04 |
| MOB kinase activator 1B | MOBKL1A | Q7L9L4 | 0.26 | 0.15 | 0.38 | 8.54E−06 | 5.43E−05 |
| Very long-chain specific acyl-CoA | ACADVL | P49748 | 0.26 | 0.15 | 0.36 | 2.34E−06 | 1.85E−05 |
| dehydrogenase, mitochondrial | | | | | | | |
| Tyrosine--tRNA ligase, cytoplasmic | YARS | P54577 | 0.26 | 0.13 | 0.39 | 1.33E−04 | 5.36E−04 |
| Serine/threonine-protein phosphatase PP1-gamma | PPP1CC | P36873 | 0.26 | 0.13 | 0.39 | 1.10E−04 | 4.58E−04 |
| catalytic subunit | | | | | | | |
| 60 kDa heat shock protein, mitochondrial | HSPD1 | P10809 | 0.26 | 0.14 | 0.38 | 1.70E−05 | 9.47E−05 |
| Tyrosine-protein kinase Lyn | LYN | P07948 | 0.26 | 0.14 | 0.37 | 1.67E−05 | 9.31E−05 |
| Importin subunit beta-1 | KPNB1 | Q14974 | 0.26 | 0.11 | 0.41 | 6.82E−04 | 2.20E−03 |
| Pyruvate kinase PKM | PKM2 | P14618 | 0.26 | 0.14 | 0.38 | 2.09E−05 | 1.12E−04 |
| [Pyruvate dehydrogenase (acetyl-transferring)] | PDK1 | Q15118 | 0.26 | 0.07 | 0.44 | 7.60E−03 | 1.89E−02 |
| kinase isozyme 1, mitochondrial | | | | | | | |
| cGMP-specific 3′,5′-cyclic phosphodiesterase | PDE5A | O76074 | 0.26 | 0.14 | 0.38 | 1.85E−05 | 1.02E−04 |
| Protein kinase C beta type (splice variant beta-II) | PRKCB | P05771 | 0.26 | 0.14 | 0.39 | 3.56E−05 | 1.75E−04 |
| Sarcolemmal membrane-associated protein | SLMAP | Q14BN4 | 0.26 | 0.06 | 0.47 | 1.25E−02 | 2.96E−02 |
| Platelet endothelial aggregation receptor | PEAR1 | Q5VY43 | 0.26 | 0.11 | 0.4 | 5.64E−04 | 1.87E−03 |
| 1: Cytoplasmic domain | | | | | | | |
| Tetratricopeptide repeat protein 1 | TTC1 | Q99614 | 0.26 | 0.14 | 0.38 | 1.19E−05 | 7.03E−05 |
| Adenine phosphoribosyltransferase | APRT | P07741 | 0.25 | 0.13 | 0.37 | 4.65E−05 | 2.20E−04 |
| Kinesin light chain 1 | KLC1 | Q07866 | 0.25 | 0.13 | 0.37 | 5.20E−05 | 2.41E−04 |
| Serine hydroxymethyltransferase, mitochondrial | SHMT2 | P34897 | 0.25 | 0.14 | 0.37 | 8.92E−06 | 5.59E−05 |
| Serine/threonine-protein kinase Nek7 | NEK7 | Q8TDX7 | 0.25 | 0.13 | 0.37 | 2.57E−05 | 1.33E−04 |
| KIF1-binding protein | KIAA1279 | Q96EK5 | 0.25 | 0.15 | 0.36 | 2.37E−06 | 1.87E−05 |
| Mitogen-activated protein kinase 10 | MAPK10 | P53779 | 0.25 | 0.14 | 0.37 | 1.64E−05 | 9.19E−05 |
| Phosphomannomutase 2 | PMM2 | O15305 | 0.25 | 0.12 | 0.37 | 8.01E−05 | 3.50E−04 |
| Endophilin-A3 | SH3GL3 | Q99963 | 0.25 | 0.14 | 0.37 | 1.59E−05 | 9.00E−05 |
| Protein kinase C alpha type | PRKCA | P17252 | 0.25 | 0.14 | 0.36 | 1.14E−05 | 6.89E−05 |
| Ras-related C3 botulinum toxin substrate 1 | RAC1 | P63000 | 0.25 | 0.13 | 0.38 | 5.74E−05 | 2.63E−04 |
| Carbonic anhydrase 13 | CA13 | Q8N1Q1 | 0.25 | 0.14 | 0.37 | 1.67E−05 | 9.33E−05 |
| Caspase-3 | CASP3 | P42574 | 0.25 | 0.12 | 0.37 | 1.01E−04 | 4.26E−04 |
| Serine/threonine-protein kinase PAK 6 | PAK6 | Q9NQU5 | 0.25 | 0.15 | 0.35 | 1.33E−06 | 1.13E−05 |
| 6-phosphogluconate dehydrogenase, | PGD | P52209 | 0.25 | 0.12 | 0.39 | 1.67E−04 | 6.54E−04 |
| decarboxylating | | | | | | | |
| Leukocyte immunoglobulin-like receptor | LILRA3 | Q8N6C8 | 0.25 | 0.07 | 0.43 | 7.90E−03 | 1.96E−02 |
| subfamily A member 3 | | | | | | | |
| Protein G6b: Isoform A, Extracellular domain | G6B | O95866 | 0.25 | 0.12 | 0.39 | 1.30E−04 | 5.28E−04 |
| CUGBP Elav-like family member 2 | CELF2 | O95319 | 0.25 | 0.04 | 0.45 | 1.88E−02 | 4.23E−02 |
| Protein MRVI1 | MRVI1 | Q9Y6F6 | 0.25 | 0.11 | 0.39 | 5.44E−04 | 1.81E−03 |
| Synaptotagmin-like protein 4: Ca2+-dependent | SYTL4 | Q96C24 | 0.25 | 0.14 | 0.36 | 1.55E−05 | 8.79E−05 |
| membrane-targeting module 1 | | | | | | | |
| Aspartate--tRNA ligase, mitochondrial | DARS2 | Q6PI48 | 0.24 | 0.15 | 0.33 | 5.51E−07 | 5.43E−06 |
| Rho GTPase-activating protein 45 | HMHA1 | Q92619 | 0.24 | 0.13 | 0.36 | 3.27E−05 | 1.64E−04 |
| Ras-related protein Rab-6B | RAB6B | Q9NRW1 | 0.24 | 0.12 | 0.36 | 1.24E−04 | 5.05E−04 |
| Cysteine and glycine-rich protein 1 | CSRP1 | P21291 | 0.24 | 0.1 | 0.38 | 9.10E−04 | 2.85E−03 |
| Microtubule-associated protein RP/EB family | MAPRE2 | Q15555 | 0.24 | 0.13 | 0.34 | 9.93E−06 | 6.12E−05 |
| member 2 | | | | | | | |
| Medium-chain specific acyl-CoA dehydrogenase, | ACADM | P11310 | 0.24 | 0.12 | 0.37 | 1.75E−04 | 6.77E−04 |
| mitochondrial | | | | | | | |
| Cdc42-interacting protein 4 | TRIP10 | Q15642 | 0.24 | 0.14 | 0.35 | 1.17E−05 | 6.96E−05 |
| Hydroxyacyl-coenzyme A dehydrogenase, | HADH | Q16836 | 0.24 | 0.14 | 0.35 | 9.06E−06 | 5.67E−05 |
| mitochondrial | | | | | | | |
| AMP deaminase 2 | AMPD2 | Q01433 | 0.24 | 0.13 | 0.35 | 2.63E−05 | 1.36E−04 |
| Leucine-rich repeat-containing protein 59 | LRRC59 | Q96AG4 | 0.24 | 0.14 | 0.34 | 2.37E−06 | 1.87E−05 |
| nucleosome assembly protein 1-like 4 | NAP1L4 | Q99733 | 0.24 | 0.12 | 0.37 | 8.70E−05 | 3.75E−04 |
| Tyrosine-protein kinase BTK | BTK | Q06187 | 0.24 | 0.12 | 0.36 | 1.15E−04 | 4.75E−04 |
| Integrin alpha-IIb: beta-3 complex | ITGA2B ITGB3 | P05106\|P08514 | 0.24 | 0.1 | 0.37 | 4.33E−04 | 1.49E−03 |
| Ras-related protein Rab-3C | RAB3C | Q96E17 | 0.24 | 0.12 | 0.35 | 3.37E−05 | 1.67E−04 |
| MAP kinase-activated protein kinase 3 | MAPKAPK3 | Q16644 | 0.24 | 0.1 | 0.38 | 5.91E−04 | 1.95E−03 |
| Tyrosine-protein kinase Fer | FER | P16591 | 0.24 | 0.13 | 0.35 | 2.30E−05 | 1.22E−04 |
| Sphingosine kinase 1 | SPHK1 | Q9NYA1 | 0.24 | 0.13 | 0.35 | 2.02E−05 | 1.09E−04 |
| Zymogen granule membrane protein 16 | ZG16 | O60844 | 0.24 | 0.18 | 0.3 | 2.19E−14 | 9.16E−13 |
| Transcription factor A, mitochondrial | TFAM | Q00059 | 0.24 | 0.13 | 0.35 | 1.24E−05 | 7.29E−05 |
| Growth factor receptor-bound protein 10 | GRB10 | Q13322 | 0.23 | 0.12 | 0.33 | 2.93E−05 | 1.50E−04 |
| Dual adapter for phosphotyrosine and 3- | DAPP1 | Q9UN19 | 0.23 | 0.12 | 0.34 | 7.43E−05 | 3.28E−04 |
| phosphotyrosine and 3-phosphoinositide | | | | | | | |
| Clathrin interactor 1 | CLINT1 | Q14677 | 0.23 | 0.11 | 0.35 | 2.56E−04 | 9.45E−04 |
| C-1-tetrahydrofolate synthase, cytoplasmic | MTHFD1 | P11586 | 0.23 | 0.05 | 0.41 | 1.09E−02 | 2.60E−02 |
| Beta-arrestin-1 | ARRB1 | P49407 | 0.23 | 0.12 | 0.34 | 3.54E−05 | 1.75E−04 |
| Adenosylhomocysteinase 2 | AHCYL1 | O43865 | 0.23 | 0.1 | 0.36 | 6.10E−04 | 2.00E−03 |
| 2,4-dienoyl-CoA reductase, mitochondrial | DECR1 | Q16698 | 0.23 | 0.11 | 0.34 | 1.17E−04 | 4.80E−04 |
| Acyl-coenzyme A thioesterase 13 | ACOT13 | Q9NPJ3 | 0.23 | 0.12 | 0.33 | 2.34E−05 | 1.23E−04 |
| Galectin-related protein | HSPC159 | Q3ZCW2 | 0.23 | 0.1 | 0.35 | 4.32E−04 | 1.48E−03 |
| Methylmalonate-semialdehyde dehydrogenase | ALDH6A1 | Q02252 | 0.23 | 0.09 | 0.37 | 1.26E−03 | 3.81E−03 |
| [acylating], mitochondrial | | | | | | | |
| GMP reductase 1 | GMPR | P36959 | 0.23 | 0.15 | 0.31 | 3.19E−08 | 4.27E−07 |
| Alpha-1-syntrophin | SNTA1 | Q13424 | 0.23 | 0.11 | 0.35 | 9.30E−05 | 3.96E−04 |
| Methionine aminopeptidase 1 | METAP1 | P53582 | 0.23 | 0.11 | 0.34 | 1.15E−04 | 4.75E−04 |
| CAMP-dependent protein kinase catalytic subunit | PRKACA | P17612 | 0.23 | 0.08 | 0.37 | 1.88E−03 | 5.41E−03 |
| alpha | | | | | | | |
| Eukaryotic translation initiation factor 4 gamma 2 | EIF4G2 | P78344 | 0.23 | 0.12 | 0.33 | 1.66E−05 | 9.26E−05 |
| Peptidyl-prolyl cis-trans isomerase D | PPID | Q08752 | 0.23 | 0.12 | 0.33 | 2.26E−05 | 1.20E−04 |
| Protein O-glucosyltransferase 1 | POGLUT1 | Q8NBL1 | 0.23 | 0.1 | 0.36 | 4.23E−04 | 1.45E−03 |
| Translation initiation factor IF-3, mitochondrial | MTIF3 | Q9H2K0 | 0.23 | 0.13 | 0.33 | 1.19E−05 | 7.05E−05 |
| Pleckstrin | PLEK | P08567 | 0.23 | 0.11 | 0.36 | 3.04E−04 | 1.09E−03 |
| Dual specificity phosphatase 28 | DUSP28 | Q4G0W2 | 0.23 | 0.11 | 0.35 | 2.21E−04 | 8.32E−04 |
| Tyrosine-protein kinase SYK: Src Homology | SYK | P43405 | 0.22 | 0.09 | 0.34 | 6.80E−04 | 2.20E−03 |
| domain | | | | | | | |
| Glycogen phosphorylase, liver form | PYGL | P06737 | 0.22 | 0.04 | 0.4 | 1.55E−02 | 3.57E−02 |
| Adenylyl cyclase-associated protein 1 | CAP1 | Q01518 | 0.22 | 0.1 | 0.35 | 4.48E−04 | 1.53E−03 |
| Serine hydroxymethyltransferase, cytosolic | SHMT1 | P34896 | 0.22 | 0.07 | 0.36 | 4.16E−03 | 1.11E−02 |
| Oligophrenin-1 | OPHN1 | O60890 | 0.22 | 0.08 | 0.37 | 1.85E−03 | 5.34E−03 |
| Carnitine O-acetyltransferase | CRAT | P43155 | 0.22 | 0.1 | 0.33 | 1.74E−04 | 6.75E−04 |
| Tropomodulin-2 | TMOD2 | Q9NZR1 | 0.22 | 0.06 | 0.38 | 8.82E−03 | 2.17E−02 |
| Prostaglandin reductase 3 | ZADH2 | Q8N4Q0 | 0.22 | 0.11 | 0.33 | 9.08E−05 | 3.89E−04 |
| Ras-related protein Rab-32 | RAB32 | Q13637 | 0.22 | 0.13 | 0.32 | 1.29E−06 | 1.09E−05 |
| Low density lipoprotein receptor adapter protein | LDLRAP1 | Q5SW96 | 0.22 | 0.12 | 0.33 | 3.12E−05 | 1.57E−04 |
| 1 | | | | | | | |
| Ras-related protein Rab-1B | RAB1B | Q9H0U4 | 0.22 | 0.12 | 0.33 | 5.00E−05 | 2.33E−04 |
| Tyrosine-protein kinase Fyn | FYN | P06241 | 0.22 | 0.06 | 0.38 | 6.65E−03 | 1.70E−02 |
| Peptidyl-prolyl cis-trans isomerase F, | PPIF | P30405 | 0.22 | 0.11 | 0.33 | 1.43E−04 | 5.71E−04 |
| mitochondrial | | | | | | | |
| Beta-1-syntrophin | SNTB1 | Q13884 | 0.22 | 0.08 | 0.36 | 1.83E−03 | 5.29E−03 |
| Mesencephalic astrocyte-derived neurotrophic | MANF | P55145 | 0.22 | 0.11 | 0.32 | 3.43E−05 | 1.70E−04 |
| factor | | | | | | | |
| Ubiquitin-associated and SH3 domain-containing | UBASH3B | Q8TF42 | 0.21 | 0.11 | 0.31 | 4.27E−05 | 2.06E−04 |
| protein B | | | | | | | |
| Type II inositol 1,4,5-trisphosphate 5- | INPP5B | P32019 | 0.21 | 0.11 | 0.31 | 2.98E−05 | 1.52E−04 |
| phosphatase | | | | | | | |
| Neuromodulin | GAP43 | P17677 | 0.21 | 0.12 | 0.31 | 1.62E−05 | 9.10E−05 |
| Bifunctional methylenetetrahydrofolate | MTHFD2 | P13995 | 0.21 | 0.08 | 0.34 | 1.29E−03 | 3.89E−03 |
| dehydrogenase/cyclohydrolase, mitochondrial | | | | | | | |
| SH3 domain-binding glutamic acid-rich-like | SH3BGRL2 | Q9UJC5 | 0.21 | 0.11 | 0.3 | 4.64E−05 | 2.20E−04 |
| protein 2 | | | | | | | |
| Human Chorionic Gonadotropin | CGA CGB | P0DN87\|P0DN86\|P01215 | 0.21 | 0.09 | 0.34 | 5.30E−04 | 1.78E−03 |
| Proto-oncogene tyrosine-protein kinase Src | SRC | P12931 | 0.21 | 0.1 | 0.32 | 1.82E−04 | 7.02E−04 |
| isoform 2 | | | | | | | |
| 39S ribosomal protein L14, mitochondrial | MRPL14 | Q6P1L8 | 0.21 | 0.05 | 0.36 | 8.89E−03 | 2.18E−02 |
| Cell division control protein 42 homolog | CDC42 | P60953 | 0.21 | 0.1 | 0.32 | 1.68E−04 | 6.57E−04 |
| Integrin-linked protein kinase | ILK | Q13418 | 0.2 | 0.09 | 0.31 | 2.60E−04 | 9.59E−04 |
| Protein unc-45 homolog A | UNC45A | Q9H3U1 | 0.2 | 0.09 | 0.32 | 5.68E−04 | 1.88E−03 |
| PRA1 family protein 3 | ARL6IP5 | O75915 | 0.2 | 0.09 | 0.3 | 2.29E−04 | 8.60E−04 |
| T-complex protein 1 subunit eta | CCT7 | Q99832 | 0.2 | 0.04 | 0.35 | 1.49E−02 | 3.45E−02 |
| Isovaleryl-CoA dehydrogenase, mitochondrial | IVD | P26440 | 0.2 | 0.08 | 0.33 | 1.68E−03 | 4.90E−03 |
| Protein disulfide-isomerase A5 | PDIA5 | Q14554 | 0.2 | 0.08 | 0.31 | 5.82E−04 | 1.92E−03 |
| Sulfotransferase 1A3 | SULT1A3 | P0DMM9 | 0.19 | 0.08 | 0.29 | 4.52E−04 | 1.54E−03 |
| Proto-oncogene tyrosine-protein kinase Src | SRC | P12931 | 0.19 | 0.1 | 0.29 | 4.15E−05 | 2.00E−04 |
| Enoyl-CoA hydratase, mitochondrial | ECHS1 | P30084 | 0.19 | 0.06 | 0.32 | 5.13E−03 | 1.34E−02 |
| Fibroblast growth factor 2 | FGF2 | P09038 | 0.19 | 0.04 | 0.35 | 1.60E−02 | 3.66E−02 |
| Phosphoglycerate mutase 1 | PGAM1 | P18669 | 0.19 | 0.09 | 0.28 | 9.55E−05 | 4.05E−04 |
| Alpha-actinin-1 | ACTN1 | P12814 | 0.18 | 0.09 | 0.28 | 2.03E−04 | 7.74E−04 |
| Sialic acid-binding Ig-like lectin 5 | SIGLEC5 | O15389 | 0.17 | 0.02 | 0.32 | 2.23E−02 | 4.90E−02 |
| GTP-binding protein SAR1a | SAR1A | Q9NR31 | 0.17 | 0.09 | 0.26 | 7.63E−05 | 3.35E−04 |
| Glucose-6-phosphate 1-dehydrogenase | G6PD | P11413 | 0.17 | 0.05 | 0.29 | 4.52E−03 | 1.19E−02 |
| Maleylacetoacetate isomerase | GSTZ1 | O43708 | 0.16 | 0.04 | 0.27 | 9.30E−03 | 2.27E−02 |
| Sialic acid-binding Ig-like lectin 9 | SIGLEC9 | Q9Y336 | 0.12 | 0.03 | 0.22 | 1.30E−02 | 3.06E−02 |
| Transcobalamin-2 | TCN2 | P20062 | −0.07 | −0.12 | −0.02 | 1.08E−02 | 2.58E−02 |
| Histo-blood group ABO system transferase | ABO | P16442 | −0.1 | −0.16 | −0.03 | 6.89E−03 | 1.74E−02 |
| Creatine kinase M-type: Creatine kinase B-type | CKB CKM | P06732\|P12277 | −0.21 | −0.38 | −0.05 | 1.24E−02 | 2.94E−02 |
| heterodimer | | | | | | | |
| Ferritin light chain | FTL | P02792 | −0.24 | −0.4 | −0.08 | 4.11E−03 | 1.10E−02 |
| Teratocarcinoma-derived growth factor 1 | TDGF1 | P13385 | −0.24 | −0.4 | −0.08 | 3.68E−03 | 9.95E−03 |
| Ferritin | FTH1 FTL | P02792\|P02794 | −0.26 | −0.42 | −0.09 | 2.26E−03 | 6.39E−03 |
| Insulin-like growth factor-binding protein 1 | IGFBP1 | P08833 | −0.27 | −0.43 | −0.11 | 7.19E−04 | 2.31E−03 |
| Tapasin-related protein | TAPBPL | Q9BX59 | −0.27 | −0.4 | −0.15 | 2.43E−05 | 1.27E−04 |
| Tumor protein p53-inducible protein 11 | TP53I11 | O14683 | −0.28 | −0.47 | −0.08 | 5.44E−03 | 1.42E−02 |
| Ornithine carbamoyltransferase, mitochondrial | OTC | P00480 | −0.29 | −0.47 | −0.1 | 2.01E−03 | 5.73E−03 |
| Neurexin-1 | NRXN1 | Q9ULB1 | −0.31 | −0.56 | −0.06 | 1.63E−02 | 3.74E−02 |
| Pancreatic lipase-related protein 2 | PNLIPRP2 | P54317 | −0.32 | −0.54 | −0.11 | 3.27E−03 | 8.94E−03 |
| Fibronectin | FN1 | P02751 | −0.32 | −0.54 | −0.1 | 3.79E−03 | 1.02E−02 |
| Aflatoxin B1 aldehyde reductase member 3 | AKR7A3 | O95154 | −0.33 | −0.54 | −0.13 | 1.61E−03 | 4.71E−03 |
| Thyroid Stimulating Hormone | CGA TSHB | P01222\|P01215 | −0.34 | −0.63 | −0.05 | 2.10E−02 | 4.64E−02 |
| Tumor necrosis factor receptor superfamily | NGFR | P08138 | −0.34 | −0.6 | −0.08 | 9.54E−03 | 2.31E−02 |
| member 16 | | | | | | | |
| Phosphoserine aminotransferase | PSAT1 | Q9Y617 | −0.35 | −0.58 | −0.12 | 2.53E−03 | 7.07E−03 |
| Lactase-phlorizin hydrolase | LCT | P09848 | −0.35 | −0.52 | −0.18 | 6.00E−05 | 2.72E−04 |
| Chymotrypsinogen B2 | CTRB2 | Q6GPI1 | −0.36 | −0.54 | −0.19 | 6.22E−05 | 2.81E−04 |
| Chondrocalcin | COL2A1 | P02458 | −0.37 | −0.6 | −0.14 | 1.58E−03 | 4.64E−03 |
| Odorant-binding protein 2b | OBP2B | Q9NPH6 | −0.38 | −0.6 | −0.15 | 9.16E−04 | 2.86E−03 |
| MAP kinase-activated protein kinase 5 | MAPKAPK5 | Q8IW41 | −0.39 | −0.63 | −0.16 | 1.02E−03 | 3.15E−03 |
| Interleukin-17 receptor A | IL17RA | Q96F46 | −0.4 | −0.69 | −0.11 | 6.47E−03 | 1.65E−02 |
| N-acylethanolamine-hydrolyzing acid amidase | NAAA | Q02083 | −0.4 | −0.63 | −0.17 | 8.04E−04 | 2.55E−03 |
| Inactive gamma-glutamyltranspeptidase 2 | GGT2 | P36268 | −0.4 | −0.73 | −0.07 | 1.68E−02 | 3.83E−02 |
| Ephrin type-A receptor 1 | EPHA1 | P21709 | −0.41 | −0.71 | −0.1 | 8.88E−03 | 2.18E−02 |
| Protein eyes shut homolog | EYS | Q5T1H1 | −0.41 | −0.75 | −0.08 | 1.59E−02 | 3.64E−02 |
| Alcohol dehydrogenase 4 | ADH4 | P08319 | −0.42 | −0.65 | −0.19 | 3.12E−04 | 1.11E−03 |
| Carboxypeptidase Q | CPQ | Q9Y646 | −0.42 | −0.79 | −0.06 | 2.25E−02 | 4.94E−02 |
| Lymphoid-restricted membrane protein | LRMP | Q12912 | −0.43 | −0.72 | −0.14 | 3.84E−03 | 1.04E−02 |
| Cystatin-S | CST4 | P01036 | −0.44 | −0.7 | −0.18 | 8.13E−04 | 2.57E−03 |
| Collagen alpha-2(XI) chain | COL11A2 | P13942 | −0.46 | −0.79 | −0.13 | 6.48E−03 | 1.66E−02 |
| Selenoprotein S | VIMP | Q9BQE4 | −0.46 | −0.83 | −0.09 | 1.50E−02 | 3.47E−02 |
| Oral cancer-overexpressed protein 1 | ORAOV1 | Q8WV07 | −0.46 | −0.78 | −0.15 | 4.10E−03 | 1.10E−02 |
| Prolactin-inducible protein | PIP | P12273 | −0.46 | −0.64 | −0.27 | 1.24E−06 | 1.07E−05 |
| Apolipoprotein E | APOE | P02649 | −0.47 | −0.74 | −0.19 | 8.94E−04 | 2.80E−03 |
| Aminoacylase-1 | ACY1 | Q03154 | −0.47 | −0.71 | −0.23 | 1.30E−04 | 5.28E−04 |
| Leukocyte immunoglobulin-like receptor | LILRB1 | Q8NHL6 | −0.47 | −0.66 | −0.28 | 1.24E−06 | 1.07E−05 |
| subfamily B member 1 | | | | | | | |
| Cystatin-SN | CST1 | P01037 | −0.47 | −0.79 | −0.15 | 4.51E−03 | 1.19E−02 |
| Apolipoprotein C-III | APOC3 | P02656 | −0.47 | −0.68 | −0.26 | 1.08E−05 | 6.52E−05 |
| Apolipoprotein A-V | APOA5 | Q6Q788 | −0.48 | −0.77 | −0.19 | 1.17E−03 | 3.56E−03 |
| Insulin receptor | INSR | P06213 | −0.48 | −0.87 | −0.09 | 1.66E−02 | 3.80E−02 |
| Ephrin-B3: Cytoplasmic domain | EFNB3 | Q15768 | −0.48 | −0.81 | −0.15 | 4.12E−03 | 1.10E−02 |
| Protocadherin-9 | PCDH9 | Q9HC56 | −0.49 | −0.89 | −0.09 | 1.68E−02 | 3.83E−02 |
| Protein O-mannose kinase | POMK | Q9H5K3 | −0.49 | −0.86 | −0.11 | 1.18E−02 | 2.80E−02 |
| Interleukin-27 subunit beta | EBI3 | Q14213 | −0.49 | −0.76 | −0.23 | 2.99E−04 | 1.07E−03 |
| Alpha-amylase 2B | AMY2B | P19961 | −0.49 | −0.83 | −0.16 | 4.22E−03 | 1.12E−02 |
| Coatomer subunit epsilon | COPE | O14579 | −0.49 | −0.81 | −0.18 | 2.11E−03 | 6.01E−03 |
| Insulin-like growth factor I | IGF1 | P05019 | −0.49 | −0.89 | −0.1 | 1.44E−02 | 3.35E−02 |
| Interleukin-1 receptor type 1 | IL1R1 | P14778 | −0.5 | −0.89 | −0.1 | 1.49E−02 | 3.44E−02 |
| Neurexophilin-1 | NXPH1 | P58417 | −0.5 | −0.93 | −0.07 | 2.23E−02 | 4.90E−02 |
| Prostate-specific antigen | KLK3 | P07288 | −0.5 | −0.64 | −0.37 | 5.37E−13 | 1.78E−11 |
| Axin-2 | AXIN2 | Q9Y2T1 | −0.51 | −0.93 | −0.08 | 1.90E−02 | 4.25E−02 |
| Cathepsin E | CTSE | P14091 | −0.51 | −0.94 | −0.09 | 1.70E−02 | 3.86E−02 |
| MAM domain-containing | MDGA2 | Q7Z553 | −0.51 | −0.84 | −0.18 | 2.48E−03 | 6.97E−03 |
| glycosylphosphatidylinositol anchor protein 2 | | | | | | | |
| Neutral ceramidase | ASAH2 | Q9NR71 | −0.51 | −0.92 | −0.11 | 1.36E−02 | 3.18E−02 |
| Catalase | CAT | P04040 | −0.51 | −0.79 | −0.22 | 4.87E−04 | 1.65E−03 |
| N-acetylated-alpha-linked acidic dipeptidase 2 | NAALAD2 | Q9Y3Q0 | −0.51 | −0.85 | −0.16 | 4.00E−03 | 1.07E−02 |
| Protein LEG1 homolog | LEG1 | Q6P5S2 | −0.52 | −0.72 | −0.32 | 3.76E−07 | 3.99E−06 |
| Cathepsin B | CTSB | P07858 | −0.52 | −0.93 | −0.11 | 1.26E−02 | 2.97E−02 |
| Cysteine-rich secretory protein 2 | CRISP2 | P16562 | −0.52 | −0.84 | −0.2 | 1.55E−03 | 4.56E−03 |
| Zinc finger protein 180 | ZNF180 | Q9UJW8 | −0.53 | −0.97 | −0.09 | 1.89E−02 | 4.24E−02 |
| Myoglobin | MB | P02144 | −0.53 | −0.93 | −0.13 | 8.97E−03 | 2.20E−02 |
| Cystatin-SA | CST2 | P09228 | −0.53 | −0.77 | −0.29 | 1.19E−05 | 7.05E−05 |
| SLIT and NTRK-like protein 5 | SLITRK5 | O94991 | −0.53 | −0.96 | −0.1 | 1.54E−02 | 3.54E−02 |
| Cysteine-rich with EGF-like domain protein 1 | CRELD1 | Q96HD1 | −0.53 | −0.88 | −0.17 | 3.38E−03 | 9.21E−03 |
| Opioid-binding protein/cell adhesion molecule | OPCML | Q14982 | −0.54 | −0.97 | −0.11 | 1.31E−02 | 3.08E−02 |
| Importin subunit alpha-1 | KPNA2 | P52292 | −0.54 | −0.93 | −0.16 | 6.02E−03 | 1.55E−02 |
| Cytochrome P450 3A4 | CYP3A4 | P08684 | −0.54 | −0.98 | −0.09 | 1.87E−02 | 4.20E−02 |
| Plasma serine protease inhibitor | SERPINA5 | P05154 | −0.54 | −0.94 | −0.13 | 8.98E−03 | 2.20E−02 |
| Tyrosine-protein kinase ZAP-70 | ZAP70 | P43403 | −0.54 | −0.85 | −0.23 | 6.88E−04 | 2.22E−03 |
| Retroviral-like aspartic protease 1 | ASPRV1 | Q53RT3 | −0.55 | −0.98 | −0.13 | 1.10E−02 | 2.62E−02 |
| Thioredoxin-interacting protein | TXNIP | Q9H3M7 | −0.55 | −1.01 | −0.09 | 1.99E−02 | 4.44E−02 |
| TGF-beta receptor type-2 | TGFBR2 | P37173 | −0.55 | −0.95 | −0.15 | 6.71E−03 | 1.71E−02 |
| Leucine-rich repeat and immunoglobulin-like | LINGO1 | Q96FE5 | −0.55 | −0.97 | −0.13 | 9.95E−03 | 2.40E−02 |
| domain-containing nogo receptor-interacting | | | | | | | |
| protein 1 | | | | | | | |
| Extracellular glycoprotein lacritin | LACRT | Q9GZZ8 | −0.55 | −0.78 | −0.32 | 3.04E−06 | 2.27E−05 |
| Glycosyltransferase-like protein LARGE1 | LARGE | O95461 | −0.55 | −0.99 | −0.12 | 1.19E−02 | 2.83E−02 |
| DCN1-like protein 5 | DCUN1D5 | Q9BTE7 | −0.55 | −0.94 | −0.17 | 4.41E−03 | 1.16E−02 |
| Plexin domain-containing protein 1 | PLXDC1 | Q8IUK5 | −0.55 | −1.01 | −0.09 | 2.01E−02 | 4.49E−02 |
| Zinc finger protein 41 | ZNF41 | P51814 | −0.56 | −0.98 | −0.15 | 7.81E−03 | 1.94E−02 |
| Sphingomyelin phosphodiesterase | SMPD1 | P17405 | −0.56 | −0.9 | −0.21 | 1.47E−03 | 4.32E−03 |
| Trypsin-1 | PRSS1 | P07477 | −0.56 | −0.87 | −0.26 | 2.52E−04 | 9.31E−04 |
| Integrin alpha-V: beta-5 complex | ITGAV ITGB5 | P18084\|P06756 | −0.56 | −0.91 | −0.21 | 1.90E−03 | 5.46E−03 |
| Junctophilin-1 | JPH1 | Q9HDC5 | −0.56 | −1 | −0.11 | 1.38E−02 | 3.22E−02 |
| Alcohol dehydrogenase class 4 mu/sigma chain | ADH7 | P40394 | −0.57 | −0.95 | −0.2 | 2.77E−03 | 7.67E−03 |
| Pikachurin | EGFLAM | Q63HQ2 | −0.57 | −1.02 | −0.11 | 1.49E−02 | 3.45E−02 |
| Transmembrane protein 132D | TMEM132D | Q14C87 | −0.57 | −0.99 | −0.16 | 6.56E−03 | 1.67E−02 |
| DCN1-like protein 2 | DCUN1D2 | Q6PH85 | −0.57 | −1.05 | −0.09 | 1.96E−02 | 4.38E−02 |
| Plexin-C1 | PLXNC1 | O60486 | −0.57 | −0.98 | −0.16 | 6.71E−03 | 1.71E−02 |
| Adhesion G protein-coupled receptor F5 | ADGRF5 | Q8IZF2 | −0.57 | −0.87 | −0.26 | 2.73E−04 | 9.98E−04 |
| Kunitz-type protease inhibitor 3 | SPINT3 | P49223 | −0.57 | −0.83 | −0.31 | 2.00E−05 | 1.08E−04 |
| Alcohol dehydrogenase 1B | ADH1B | P00325 | −0.57 | −1.06 | −0.09 | 2.07E−02 | 4.60E−02 |
| Netrin receptor UNC5D | UNC5D | Q6UXZ4 | −0.58 | −0.96 | −0.2 | 3.12E−03 | 8.56E−03 |
| Protein timeless homolog | TIMELESS | Q9UNS1 | −0.58 | −1.05 | −0.11 | 1.47E−02 | 3.41E−02 |
| Protein DEPP | DEPP | Q9NTK1 | −0.58 | −0.91 | −0.25 | 5.57E−04 | 1.85E−03 |
| Tryptophan 2,3-dioxygenase | TDO2 | P48775 | −0.58 | −1.01 | −0.15 | 8.62E−03 | 2.12E−02 |
| Glycerol-3-phosphate dehydrogenase 1-like | GPD1L | Q8N335 | −0.59 | −1.02 | −0.16 | 7.17E−03 | 1.80E−02 |
| protein | | | | | | | |
| Kin of IRRE-like protein 2 | KIRREL2 | Q6UWL6 | −0.59 | −0.93 | −0.24 | 7.79E−04 | 2.48E−03 |
| Peptidyl-prolyl cis-trans isomerase G | PPIG | Q13427 | −0.59 | −0.95 | −0.23 | 1.23E−03 | 3.72E−03 |
| Oncostatin-M | OSM | P13725 | −0.59 | −0.98 | −0.2 | 3.07E−03 | 8.44E−03 |
| Nicotinamide phosphoribosyltransferase | NAMPT | P43490 | −0.59 | −1.02 | −0.17 | 6.40E−03 | 1.64E−02 |
| NADH dehydrogenase [ubiquinone] 1 beta | NDUFB4 | O95168 | −0.6 | −0.95 | −0.25 | 7.22E−04 | 2.32E−03 |
| subcomplex subunit 4 | | | | | | | |
| Dual specificity protein kinase CLK2 | CLK2 | P49760 | −0.6 | −1.02 | −0.18 | 5.00E−03 | 1.31E−02 |
| Aspartoacylase | ASPA | P45381 | −0.6 | −1.08 | −0.11 | 1.53E−02 | 3.52E−02 |
| Glyceraldehyde-3-phosphate dehydrogenase, | GAPDHS | O14556 | −0.6 | −0.96 | −0.23 | 1.30E−03 | 3.92E−03 |
| testis-specific | | | | | | | |
| BRCA1-associated ATM activator 1 | BRAT1 | Q6PJG6 | −0.61 | −1.05 | −0.18 | 5.42E−03 | 1.41E−02 |
| Unique cartilage matrix-associated protein | UCMA | Q8WVF2 | −0.61 | −1 | −0.23 | 1.55E−03 | 4.56E−03 |
| Apolipoprotein L1 | APOL1 | O14791 | −0.61 | −1.14 | −0.09 | 2.26E−02 | 4.95E−02 |
| Ly6/PLAUR domain-containing protein 3 | LYPD3 | O95274 | −0.61 | −1.13 | −0.09 | 2.12E−02 | 4.68E−02 |
| Secreted frizzled-related protein 3 | FRZB | Q92765 | −0.61 | −1.1 | −0.11 | 1.56E−02 | 3.59E−02 |
| Peptidyl-prolyl cis-trans isomerase-like 2 | PPIL2 | Q13356 | −0.61 | −1.06 | −0.17 | 6.86E−03 | 1.74E−02 |
| Tumor necrosis factor ligand superfamily | FASLG | P48023 | −0.61 | −0.94 | −0.28 | 3.18E−04 | 1.13E−03 |
| member 6, soluble form | | | | | | | |
| Leptin receptor, soluble | LEPR | P48357 | −0.61 | −0.86 | −0.36 | 1.53E−06 | 1.28E−05 |
| Beta-Ala-His dipeptidase | CNDP1 | Q96KN2 | −0.61 | −0.83 | −0.39 | 5.24E−08 | 6.62E−07 |
| High affinity immunoglobulin epsilon receptor | FCER1A | P12319 | −0.61 | −0.99 | −0.23 | 1.56E−03 | 4.56E−03 |
| subunit alpha | | | | | | | |
| Cathepsin O | CTSO | P43234 | −0.61 | −1.08 | −0.14 | 1.03E−02 | 2.47E−02 |
| T-cell surface glycoprotein CD8 beta chain | CD8B | P10966 | −0.61 | −1.08 | −0.13 | 1.19E−02 | 2.83E−02 |
| Cathelicidin antimicrobial peptide | CAMP | P49913 | −0.61 | −0.91 | −0.3 | 1.02E−04 | 4.30E−04 |
| Alpha-1,3-mannosyl-glycoprotein 4-beta-N- | MGAT4A | Q9UM21 | −0.61 | −1.08 | −0.14 | 1.05E−02 | 2.53E−02 |
| acetylglucosaminyltransferase A | | | | | | | |
| Peptidoglycan recognition protein 3 | PGLYRP3 | Q96LB9 | −0.62 | −1.13 | −0.1 | 2.01E−02 | 4.48E−02 |
| TOMM20-like protein 1 | TOMM20L | Q6UXN7 | −0.62 | −1.13 | −0.11 | 1.67E−02 | 3.81E−02 |
| Collagen alpha-1(I) chain: C-term propeptide | COL1A1 | P02452 | −0.62 | −1.06 | −0.17 | 6.80E−03 | 1.72E−02 |
| Sulfotransferase family cytosolic 1B member 1 | SULT1B1 | O43704 | −0.62 | −1.11 | −0.12 | 1.46E−02 | 3.39E−02 |
| Adseverin | SCIN | Q9Y6U3 | −0.62 | −1.12 | −0.12 | 1.55E−02 | 3.57E−02 |
| Myomesin-3 | MYOM3 | Q5VTT5 | −0.62 | −1.1 | −0.13 | 1.31E−02 | 3.08E−02 |
| Carboxypeptidase B | CPB1 | P15086 | −0.62 | −0.88 | −0.37 | 1.53E−06 | 1.28E−05 |
| Islet amyloid polypeptide | IAPP | P10997 | −0.62 | −1.02 | −0.21 | 2.84E−03 | 7.84E−03 |
| Interleukin-37 | IL37 | Q9NZH6 | −0.62 | −1.09 | −0.15 | 9.89E−03 | 2.39E−02 |
| Fibronectin Fragment 4 | FN1 | P02751 | −0.62 | −1.04 | −0.2 | 3.50E−03 | 9.51E−03 |
| Endothelial cell-specific molecule 1 | ESM1 | Q9NQ30 | −0.62 | −1.11 | −0.12 | 1.52E−02 | 3.51E−02 |
| Ephrin type-A receptor 5 | EPHA5 | P54756 | −0.62 | −1.14 | −0.09 | 2.13E−02 | 4.71E−02 |
| Antigen-presenting glycoprotein CDId | CD1D | P15813 | −0.62 | −1.16 | −0.09 | 2.18E−02 | 4.81E−02 |
| Low-density lipoprotein receptor-related protein | LRP | Q07954 | −0.62 | −1.09 | −0.15 | 9.75E−03 | 2.36E−02 |
| 1, soluble: Extracellular domain, LDL receptor | | | | | | | |
| class A domains 11-20 | | | | | | | |
| RNA polymerase II elongation factor ELL2 | ELL2 | O00472 | −0.63 | −1.05 | −0.2 | 4.08E−03 | 1.09E−02 |
| Leucine-rich repeat transmembrane protein | FLRT3 | Q9NZU0 | −0.63 | −1.04 | −0.22 | 2.72E−03 | 7.54E−03 |
| FLRT3: Extracellular domain | | | | | | | |
| Coagulation factor XIII | F13A1 F13B | P05160\|P00488 | −0.63 | −1.14 | −0.11 | 1.66E−02 | 3.80E−02 |
| Melanoma-associated antigen 5 | MAGEA5 | P43359 | −0.63 | −1 | −0.26 | 8.55E−04 | 2.69E−03 |
| Tropomyosin beta chain | TPM2 | P07951 | −0.63 | −0.97 | −0.29 | 2.54E−04 | 9.39E−04 |
| Endoglin | ENG | P17813 | −0.63 | −1.06 | −0.2 | 4.25E−03 | 1.13E−02 |
| Leucine-rich repeat serine/threonine-protein | LRRK2 | Q5S007 | −0.64 | −1.13 | −0.15 | 9.87E−03 | 2.38E−02 |
| kinase 2 | | | | | | | |
| BPI fold-containing family A member 2 | BPIFA2 | Q96DR5 | −0.64 | −1.05 | −0.23 | 2.20E−03 | 6.24E−03 |
| Tumor necrosis factor receptor superfamily | LTBR | P36941 | −0.64 | −1.01 | −0.27 | 6.08E−04 | 2.00E−03 |
| member 3 | | | | | | | |
| Ck-beta-8-1 | CCL23 | P55773 | −0.64 | −0.97 | −0.32 | 1.14E−04 | 4.74E−04 |
| Coagulation Factor V | F5 | P12259 | −0.64 | −1.09 | −0.18 | 5.95E−03 | 1.53E−02 |
| Inactive pancreatic lipase-related protein 1 | PNLIPRP1 | P54315 | −0.64 | −0.94 | −0.35 | 1.68E−05 | 9.34E−05 |
| Pregnancy-specific beta-1-glycoprotein 5 | PSG5 | Q15238 | −0.64 | −0.93 | −0.34 | 3.25E−05 | 1.63E−04 |
| Neurexophilin-2 | NXPH2 | O95156 | −0.64 | −0.91 | −0.37 | 2.54E−06 | 1.96E−05 |
| Neuroendocrine protein 7B2 | SCG5 | P05408 | −0.65 | −1.02 | −0.29 | 4.18E−04 | 1.44E−03 |
| SLIT and NTRK-like protein 1 | SLITRK1 | Q96PX8 | −0.65 | −1.02 | −0.28 | 5.19E−04 | 1.75E−03 |
| Ribonuclease P protein subunit p30 | RPP30 | P78346 | −0.65 | −1.16 | −0.13 | 1.36E−02 | 3.19E−02 |
| Insulin-like growth factor-binding protein 5 | IGFBP5 | P24593 | −0.65 | −1.01 | −0.28 | 5.79E−04 | 1.91E−03 |
| Growth/differentiation factor 11/8 | GDF11 MSTN | O14793\|O95390 | −0.65 | −1.15 | −0.14 | 1.22E−02 | 2.88E−02 |
| Kallikrein-6 | KLK6 | Q92876 | −0.65 | −1.07 | −0.24 | 1.90E−03 | 5.45E−03 |
| Leucine-rich repeat and fibronectin type-III | LRFN5 | Q96NI6 | −0.65 | −1.12 | −0.18 | 6.30E−03 | 1.61E−02 |
| domain-containing protein 5 | | | | | | | |
| EGF-like repeat and discoidin I-like domain- | EDIL3 | O43854 | −0.65 | −0.95 | −0.35 | 1.94E−05 | 1.06E−04 |
| containing protein 3 | | | | | | | |
| Pancreatic alpha-amylase | AMY2A | P04746 | −0.66 | −1 | −0.33 | 9.20E−05 | 3.92E−04 |
| Noelin-2 | OLFM2 | O95897 | −0.66 | −1.02 | −0.3 | 2.90E−04 | 1.05E−03 |
| Interleukin-2 | IL2 | P60568 | −0.66 | −1.18 | −0.13 | 1.42E−02 | 3.31E−02 |
| Ras-related protein Rab-26 | RAB26 | Q9ULW5 | −0.66 | −1.16 | −0.17 | 8.77E−03 | 2.16E−02 |
| Neurotrimin | NTM | Q9P121 | −0.67 | −1.08 | −0.26 | 1.29E−03 | 3.89E−03 |
| Transmembrane protein 154 | TMEM154 | Q6P9G4 | −0.67 | −1.23 | −0.1 | 2.15E−02 | 4.75E−02 |
| Spindlin-1 | SPIN1 | Q9Y657 | −0.67 | −1.23 | −0.11 | 1.82E−02 | 4.10E−02 |
| 3-ketodihydrosphingosine reductase | KDSR | Q06136 | −0.67 | −1.15 | −0.2 | 5.25E−03 | 1.37E−02 |
| Transmembrane protein 106B | TMEM106B | Q9NUM4 | −0.67 | −1.2 | −0.15 | 1.20E−02 | 2.85E−02 |
| Fc receptor-like protein 4: Extracellular domain | FCRLA | Q96PJ5 | −0.67 | −0.98 | −0.36 | 2.02E−05 | 1.09E−04 |
| Beta-defensin 132 | DEFB132 | Q7Z7B7 | −0.67 | −1.1 | −0.24 | 2.49E−03 | 6.98E−03 |
| Protein Wnt-5a | WNT5A | P41221 | −0.68 | −1.01 | −0.35 | 4.86E−05 | 2.28E−04 |
| Chymotrypsinogen B | CTRB1 | P17538 | −0.68 | −0.95 | −0.4 | 1.84E−06 | 1.50E−05 |
| NADH-cytochrome b5 reductase 3 | CYB5R3 | P00387 | −0.68 | −0.97 | −0.38 | 6.95E−06 | 4.60E−05 |
| PH and SEC7 domain-containing protein 1 | PSD | A5PKW4 | −0.69 | −1.11 | −0.27 | 1.20E−03 | 3.63E−03 |
| NAD-dependent protein deacetylase sirtuin-3, | SIRT3 | Q9NTG7 | −0.69 | −1.2 | −0.18 | 7.98E−03 | 1.98E−02 |
| mitochondrial | | | | | | | |
| Nicotinate-nucleotide pyrophosphorylase | QPRT | Q15274 | −0.69 | −1.19 | −0.19 | 6.92E−03 | 1.75E−02 |
| [carboxylating] | | | | | | | |
| Sonic hedgehog protein | SHH | Q15465 | −0.69 | −1.03 | −0.34 | 1.06E−04 | 4.45E−04 |
| UDP-glucuronosyltransferase 1-8 | UGT1A8 | Q9HAW9 | −0.69 | −1.14 | −0.23 | 2.98E−03 | 8.21E−03 |
| Nuclear factor of activated T-cells, cytoplasmic 1 | NFATC1 | O95644 | −0.7 | −1.28 | −0.11 | 2.07E−02 | 4.60E−02 |
| 5′-Nucleotidase | NT5E | P21589 | −0.7 | −1.12 | −0.28 | 1.04E−03 | 3.19E−03 |
| Cell adhesion molecule 2 | CADM2 | Q8N3J6 | −0.7 | −1.15 | −0.26 | 2.04E−03 | 5.82E−03 |
| Fructose-bisphosphate aldolase B | ALDOB | P05062 | −0.7 | −0.93 | −0.46 | 8.72E−09 | 1.33E−07 |
| Low-density lipoprotein receptor-related protein | LRP4 | O75096 | −0.7 | −1.14 | −0.25 | 2.12E−03 | 6.01E−03 |
| 4 | | | | | | | |
| Glypican-5 | GPC5 | P78333 | −0.7 | −1.09 | −0.32 | 3.65E−04 | 1.28E−03 |
| Beta-defensin 106 | DEFB106A | Q8N104 | −0.7 | −1.24 | −0.17 | 1.03E−02 | 2.47E−02 |
| UDP-GlcNAc: betaGal beta-1,3-N- | B3GNT8 | Q7Z7M8 | −0.7 | −1.1 | −0.3 | 6.50E−04 | 2.11E−03 |
| acetylglucosaminyltransferase 8 | | | | | | | |
| Myotubularin-related protein 1 | MTMR1 | Q13613 | −0.71 | −1.24 | −0.17 | 9.50E−03 | 2.31E−02 |
| Lymphotoxin alpha2: beta1 | LTA LTB | Q06643\|P01374 | −0.71 | −1.28 | −0.14 | 1.48E−02 | 3.42E−02 |
| Advanced glycosylation end product-specific | AGER | Q15109 | −0.71 | −0.91 | −0.51 | 2.14E−12 | 6.51E−11 |
| receptor, soluble | | | | | | | |
| C-type lectin domain family 4 member C | CLEC4C | Q8WTT0 | −0.71 | −1.03 | −0.38 | 2.74E−05 | 1.41E−04 |
| PH and SEC7 domain-containing protein 2 | PSD2 | Q9BQI7 | −0.71 | −1.04 | −0.37 | 3.69E−05 | 1.81E−04 |
| Leucine-rich repeat-containing protein | LRRC25 | Q8N386 | −0.71 | −1.29 | −0.13 | 1.72E−02 | 3.91E−02 |
| 25: Extracellular domain | | | | | | | |
| Zinc finger protein 276 | ZNF276 | Q8N554 | −0.72 | −1.12 | −0.32 | 4.50E−04 | 1.54E−03 |
| Pancreatic triacylglycerol lipase | PNLIP | P16233 | −0.72 | −0.97 | −0.47 | 1.24E−08 | 1.83E−07 |
| DNA repair protein RAD51 homolog 1 | RAD51 | Q06609 | −0.72 | −1.07 | −0.37 | 5.94E−05 | 2.70E−04 |
| Uncharacterized protein C20orf173 | C20orf173 | Q96LM9 | −0.72 | −1.34 | −0.11 | 2.05E−02 | 4.57E−02 |
| Vascular endothelial growth factor receptor 1 | FLT1 | P17948 | −0.72 | −1.08 | −0.37 | 5.85E−05 | 2.68E−04 |
| Myelin protein zero-like protein 2 | MPZL2 | O60487 | −0.72 | −1.2 | −0.24 | 3.37E−03 | 9.20E−03 |
| Tubulointerstitial nephritis antigen-like | TINAGL1 | Q9GZM7 | −0.73 | −1.18 | −0.28 | 1.47E−03 | 4.32E−03 |
| Epididymal-specific lipocalin-10 | LCN10 | Q6JVE6 | −0.73 | −1.15 | −0.31 | 7.20E−04 | 2.31E−03 |
| Protein Wnt-3a | WNT3A | P56704 | −0.73 | −1.16 | −0.31 | 6.88E−04 | 2.22E−03 |
| Kv channel-interacting protein 1 | KCNIP1 | Q9NZI2 | −0.73 | −1.18 | −0.28 | 1.44E−03 | 4.25E−03 |
| Glycine N-methyltransferase | GNMT | Q14749 | −0.73 | −1.35 | −0.11 | 2.13E−02 | 4.70E−02 |
| Properdin | CFP | P27918 | −0.73 | −1.28 | −0.19 | 8.22E−03 | 2.04E−02 |
| Toll-like receptor 4: Lymphocyte antigen 96 | TLR4 LY96 | Q9Y6Y9\|O00206 | −0.73 | −1.26 | −0.2 | 7.01E−03 | 1.77E−02 |
| complex | | | | | | | |
| Sodium/potassium-transporting ATPase subunit | ATP1B2 | P14415 | −0.73 | −1.19 | −0.27 | 1.90E−03 | 5.45E−03 |
| beta-2 | | | | | | | |
| T-cell surface glycoprotein CD5: Cytoplasmic | CD5 | P06127 | −0.74 | −1.28 | −0.19 | 8.24E−03 | 2.04E−02 |
| domain | | | | | | | |
| Probable ATP-dependent RNA helicase DDX46 | DDX46 | Q7L014 | −0.74 | −1.27 | −0.21 | 5.89E−03 | 1.52E−02 |
| Cysteine-rich protein 1 | CRIP1 | P50238 | −0.74 | −1.14 | −0.33 | 3.39E−04 | 1.20E−03 |
| Activin receptor type-2A | ACVR2A | P27037 | −0.74 | −1.3 | −0.18 | 9.40E−03 | 2.28E−02 |
| Cytoskeleton-associated protein 2 | CKAP2 | Q8WWK9 | −0.74 | −1.27 | −0.2 | 7.09E−03 | 1.79E−02 |
| B-cell antigen receptor complex-associated | CD79B | P40259 | −0.74 | −1.27 | −0.22 | 5.81E−03 | 1.50E−02 |
| protein beta chain | | | | | | | |
| Membrane protein FAM174A | FAM174A | Q8TBP5 | −0.74 | −1.13 | −0.35 | 1.90E−04 | 7.29E−04 |
| Hemoglobin subunit delta | HBD | P02042 | −0.74 | −1.36 | −0.12 | 1.99E−02 | 4.45E−02 |
| Growth arrest-specific protein 7 | GAS7 | O60861 | −0.75 | −1.3 | −0.2 | 7.50E−03 | 1.87E−02 |
| Ribosome biogenesis protein TSR3 homolog | TSR3 | Q9UJK0 | −0.75 | −1.28 | −0.22 | 5.31E−03 | 1.38E−02 |
| Cytoskeleton-associated protein 4 | CKAP4 | Q07065 | −0.75 | −1.36 | −0.15 | 1.48E−02 | 3.42E−02 |
| Cathepsin F | CTSF | Q9UBX1 | −0.75 | −1.3 | −0.21 | 6.96E−03 | 1.76E−02 |
| ADP-ribosylation factor-like protein 2 | ARL2 | P36404 | −0.76 | −1.18 | −0.34 | 3.75E−04 | 1.31E−03 |
| Regulator of G-protein signaling 3 | RGS3 | P49796 | −0.76 | −1.3 | −0.21 | 6.87E−03 | 1.74E−02 |
| Immunoglobulin superfamily containing leucine- | ISLR2 | Q6UXK2 | −0.76 | −1.25 | −0.28 | 2.14E−03 | 6.08E−03 |
| rich repeat protein 2 | | | | | | | |
| Apolipoprotein A-IV | APOA4 | P06727 | −0.76 | −1.24 | −0.28 | 1.81E−03 | 5.24E−03 |
| Kininostatin | KNG1 | P01042 | −0.76 | −1.03 | −0.48 | 4.95E−08 | 6.32E−07 |
| DnaJ homolog subfamily C member 4: C-term | DNAJC4 | Q9NNZ3 | −0.76 | −1.37 | −0.15 | 1.41E−02 | 3.28E−02 |
| Vacuolar protein sorting-associated protein 28 | VPS28 | Q9UK41 | −0.77 | −1.22 | −0.32 | 7.44E−04 | 2.38E−03 |
| homolog | | | | | | | |
| Kunitz-type protease inhibitor 1 | SPINT1 | O43278 | −0.77 | −1.13 | −0.42 | 1.56E−05 | 8.83E−05 |
| Biglycan | BGN | P21810 | −0.77 | −1.15 | −0.39 | 8.26E−05 | 3.58E−04 |
| Protein SERAC1 | SERAC1 | Q96JX3 | −0.77 | −1.37 | −0.17 | 1.19E−02 | 2.83E−02 |
| Sepiapterin reductase | SPR | P35270 | −0.77 | −1.43 | −0.12 | 2.10E−02 | 4.66E−02 |
| Scavenger receptor class A member 5 | SCARA5 | Q6ZMJ2 | −0.78 | −1.12 | −0.44 | 6.99E−06 | 4.61E−05 |
| Thiopurine S-methyltransferase | TPMT | P51580 | −0.78 | −1.34 | −0.21 | 6.94E−03 | 1.75E−02 |
| Phosphatidate phosphatase PPAPDC1A | PPAPDC1A | Q5VZY2 | −0.78 | −1.32 | −0.25 | 4.06E−03 | 1.09E−02 |
| Glutamyl aminopeptidase | ENPEP | Q07075 | −0.78 | −1.2 | −0.36 | 2.56E−04 | 9.45E−04 |
| CCAAT/enhancer-binding protein beta | CEBPB | P17676 | −0.78 | −1.22 | −0.34 | 5.21E−04 | 1.75E−03 |
| Interferon regulatory factor 1 | IRF1 | P10914 | −0.78 | −1.4 | −0.16 | 1.41E−02 | 3.27E−02 |
| Synaptic vesicle membrane protein VAT-1 | VAT1 | Q99536 | −0.78 | −1.13 | −0.43 | 1.21E−05 | 7.14E−05 |
| homolog | | | | | | | |
| Uroplakin-3b-like protein | UPK3BL | B0FP48 | −0.78 | −1.22 | −0.34 | 4.94E−04 | 1.68E−03 |
| C-C motif chemokine 1 | CCL1 | P22362 | −0.79 | −1.2 | −0.37 | 1.84E−04 | 7.07E−04 |
| Serine/threonine-protein kinase 16 | STK16 | O75716 | −0.79 | −1.33 | −0.25 | 4.35E−03 | 1.15E−02 |
| Interferon alpha-5 | IFNA5 | P01569 | −0.79 | −1.45 | −0.13 | 1.90E−02 | 4.26E−02 |
| Single Ig IL-1-related receptor | SIGIRR | Q6IA17 | −0.79 | −1.38 | −0.2 | 8.93E−03 | 2.19E−02 |
| Proteoglycan 3 | PRG3 | Q9Y2Y8 | −0.79 | −1.12 | −0.46 | 2.79E−06 | 2.13E−05 |
| Prostaglandin E synthase 3 | PTGES3 | Q15185 | −0.8 | −1.25 | −0.36 | 4.09E−04 | 1.41E−03 |
| Insulin-like growth factor-binding protein 3 | IGFBP3 | P17936 | −0.8 | −1.26 | −0.35 | 5.23E−04 | 1.76E−03 |
| Disintegrin and metalloproteinase domain- | ADAM23 | O75077 | −0.8 | −1.23 | −0.37 | 2.53E−04 | 9.34E−04 |
| containing protein 23 | | | | | | | |
| DNA-binding protein SATB2 | SATB2 | Q9UPW6 | −0.81 | −1.39 | −0.24 | 5.56E−03 | 1.44E−02 |
| Heterogeneous nuclear ribonucleoproteins C1/C2 | HNRNPC | P07910 | −0.81 | −1.48 | −0.13 | 1.88E−02 | 4.22E−02 |
| Electron transfer flavoprotein subunit alpha, | ETFA | P13804 | −0.81 | −1.42 | −0.21 | 8.44E−03 | 2.08E−02 |
| mitochondrial | | | | | | | |
| T-cell surface glycoprotein CD3 epsilon chain | CD3E | P07766 | −0.81 | −1.34 | −0.29 | 2.17E−03 | 6.14E−03 |
| Kallikrein-7 | KLK7 | P49862 | −0.81 | −1.28 | −0.33 | 8.72E−04 | 2.74E−03 |
| Protein CEI | C5orf38 | Q86SI9 | −0.81 | −1.18 | −0.43 | 2.42E−05 | 1.27E−04 |
| Zinc transporter 3 | SLC30A3 | Q99726 | −0.81 | −1.26 | −0.37 | 3.20E−04 | 1.14E−03 |
| cGMP-dependent protein kinase 1, beta isozyme | PRKG1 | Q13976 | −0.82 | −1.46 | −0.18 | 1.19E−02 | 2.83E−02 |
| Oligodendrocyte-myelin glycoprotein | OMG | P23515 | −0.82 | −1.1 | −0.54 | 1.00E−08 | 1.51E−07 |
| NADH dehydrogenase [ubiquinone] 1 beta | NDUFB11 | Q9NX14 | −0.82 | −1.5 | −0.14 | 1.88E−02 | 4.22E−02 |
| subcomplex subunit 11, mitochondrial | | | | | | | |
| Alpha-N-acetylgalactosaminide alpha-2,6- | ST6GALNAC2 | Q9UJ37 | −0.82 | −1.29 | −0.34 | 7.84E−04 | 2.49E−03 |
| sialyltransferase 2 | | | | | | | |
| Leucine-rich repeat and transmembrane domain- | LRTM2 | Q8N967 | −0.82 | −1.47 | −0.17 | 1.39E−02 | 3.25E−02 |
| containing protein 2 | | | | | | | |
| TOM1-like protein 2 | TOM1L2 | Q6ZVM7 | −0.83 | −1.35 | −0.31 | 1.61E−03 | 4.71E−03 |
| Macrophage-stimulating protein receptor | MST1R | Q04912 | −0.83 | −1.37 | −0.29 | 2.66E−03 | 7.40E−03 |
| Plasma protease C1 inhibitor | SERPING1 | P05155 | −0.83 | −1.34 | −0.31 | 1.69E−03 | 4.91E−03 |
| Angiogenic factor with G patch and FHA | AGGF1 | Q8N302 | −0.83 | −1.34 | −0.33 | 1.22E−03 | 3.71E−03 |
| domains 1 | | | | | | | |
| Transmembrane glycoprotein NMB: Cytoplasmic | GPNMB | Q14956 | −0.83 | −1.27 | −0.38 | 2.78E−04 | 1.01E−03 |
| domain | | | | | | | |
| Galactos ylceramide sulfotransferase | GAL3ST1 | Q99999 | −0.83 | −1.23 | −0.43 | 4.22E−05 | 2.03E−04 |
| Interferon alpha/beta receptor 1 | IFNAR1 | P17181 | −0.83 | −1.32 | −0.34 | 9.01E−04 | 2.82E−03 |
| Caspase-14 | CASP14 | P31944 | −0.84 | −1.34 | −0.35 | 7.72E−04 | 2.46E−03 |
| Very low-density lipoprotein receptor | VLDLR | P98155 | −0.84 | −1.38 | −0.3 | 2.49E−03 | 6.99E−03 |
| Cartilage acidic protein 1 | CRTAC1 | Q9NQ79 | −0.84 | −1.33 | −0.34 | 9.41E−04 | 2.93E−03 |
| Serine protease inhibitor Kazal-type 5 | SPINK5 | Q9NQ38 | −0.84 | −1.33 | −0.34 | 8.59E−04 | 2.70E−03 |
| Translocon-associated protein subunit alpha | SSR1 | P43307 | −0.84 | −1.42 | −0.27 | 4.02E−03 | 1.08E−02 |
| Cytochrome c oxidase subunit 7A1, | COX7A1 | P24310 | −0.84 | −1.35 | −0.33 | 1.31E−03 | 3.93E−03 |
| mitochondrial | | | | | | | |
| P-selectin glycoprotein ligand 1: Extracellular | SELPLG | Q14242 | −0.85 | −1.26 | −0.44 | 4.79E−05 | 2.25E−04 |
| domain | | | | | | | |
| UDP-N-acetylhexosamine pyrophosphorylase | UAP1 | Q16222 | −0.85 | −1.38 | −0.31 | 1.88E−03 | 5.41E−03 |
| Interleukin-36 receptor antagonist protein | IL1F5 | Q9UBH0 | −0.85 | −1.21 | −0.49 | 3.29E−06 | 2.44E−05 |
| Brevican core protein | BCAN | Q96GW7 | −0.85 | −1.38 | −0.33 | 1.42E−03 | 4.21E−03 |
| Neural cell adhesion molecule L1 | L1CAM | P32004 | −0.85 | −1.24 | −0.46 | 2.20E−05 | 1.17E−04 |
| ADP-dependent glucokinase | ADPGK | Q9BRR6 | −0.85 | −1.49 | −0.22 | 8.52E−03 | 2.10E−02 |
| Cadherin-related family member 3 | CDHR3 | Q6ZTQ4 | −0.85 | −1.34 | −0.35 | 7.65E−04 | 2.44E−03 |
| Interferon gamma receptor 2: Extracellular | IFNGR2 | P38484 | −0.85 | −1.47 | −0.23 | 6.87E−03 | 1.74E−02 |
| domain | | | | | | | |
| Inactive serine protease 35 | PRSS35 | Q8N3Z0 | −0.85 | −1.45 | −0.25 | 5.72E−03 | 1.48E−02 |
| ADP-ribosylation factor GTPase-activating | ARFGAP1 | Q8N6T3 | −0.86 | −1.55 | −0.18 | 1.33E−02 | 3.13E−02 |
| protein 1 | | | | | | | |
| Matrilin-3 | MATN3 | O15232 | −0.86 | −1.23 | −0.48 | 8.55E−06 | 5.43E−05 |
| Desmoglein-1 | DSG1 | Q02413 | −0.86 | −1.34 | −0.37 | 5.58E−04 | 1.85E−03 |
| Lactadherin | MFGE8 | Q08431 | −0.86 | −1.2 | −0.52 | 6.66E−07 | 6.41E−06 |
| 1-phosphatidylinositol 4,5-bisphosphate | PLCG1 | P19174 | −0.86 | −1.56 | −0.17 | 1.47E−02 | 3.41E−02 |
| phosphodiesterase gamma-1 | | | | | | | |
| Serine/threonine-protein kinase pim-1 | PIM1 | P11309 | −0.86 | −1.46 | −0.26 | 4.85E−03 | 1.27E−02 |
| Cerebellin-1 | CBLN1 | P23435 | −0.86 | −1.16 | −0.57 | 1.15E−08 | 1.72E−07 |
| Recoverin | RCVRN | P35243 | −0.87 | −1.59 | −0.16 | 1.70E−02 | 3.86E−02 |
| Aprataxin | APTX | Q7Z2E3 | −0.87 | −1.33 | −0.42 | 1.78E−04 | 6.87E−04 |
| Ectodysplasin-A, secreted form | EDA | Q92838 | −0.87 | −1.39 | −0.36 | 9.49E−04 | 2.96E−03 |
| Cyclin-dependent kinase 5: Cyclin-dependent | CDK5 CDK5R1 | Q15078\|Q00535 | −0.87 | −1.25 | −0.49 | 7.50E−06 | 4.88E−05 |
| kinase 5 activator 1 complex | | | | | | | |
| Leucine-rich repeat transmembrane neuronal | LRRTM2 | O43300 | −0.87 | −1.33 | −0.42 | 1.62E−04 | 6.35E−04 |
| protein 2 | | | | | | | |
| Insulin growth factor-like family member 3 | IGFL3 | Q6UXB1 | −0.87 | −1.36 | −0.38 | 4.97E−04 | 1.68E−03 |
| Complement C1q tumor necrosis factor-related | C1QTNF5 | Q9BXJ0 | −0.87 | −1.46 | −0.29 | 3.56E−03 | 9.65E−03 |
| protein 5 | | | | | | | |
| Alpha-hemoglobin-stabilizing protein | AHSP | Q9NZD4 | −0.87 | −1.41 | −0.33 | 1.65E−03 | 4.80E−03 |
| Secretory phospholipase A2 receptor | PLA2R1 | Q13018 | −0.88 | −1.25 | −0.52 | 2.62E−06 | 2.02E−05 |
| BRCA1-associated RING domain protein 1 | BARD1 | Q99728 | −0.88 | −1.48 | −0.27 | 4.32E−03 | 1.15E−02 |
| Platelet-derived growth factor receptor beta | PDGFRB | P09619 | −0.88 | −1.19 | −0.58 | 1.75E−08 | 2.49E−07 |
| Ig Kappa chain V-I region HK102- like | LOC652493 | P01602 | −0.88 | −1.49 | −0.26 | 5.01E−03 | 1.31E−02 |
| WAP four-disulfide core domain protein 5 | WFDC5 | Q8TCV5 | −0.88 | −1.54 | −0.22 | 9.23E−03 | 2.25E−02 |
| Signal peptide, CUB and EGF-like domain- | SCUBE1 | Q8IWY4 | −0.88 | −1.12 | −0.65 | 4.75E−13 | 1.59E−11 |
| containing protein 1 | | | | | | | |
| Fructose-bisphosphate aldolase C | ALDOC | P09972 | −0.88 | −1.32 | −0.44 | 8.87E−05 | 3.80E−04 |
| GTP cyclohydrolase 1 | GCH1 | P30793 | −0.89 | −1.46 | −0.31 | 2.50E−03 | 7.01E−03 |
| Growth factor receptor-bound protein 7 | GRB7 | Q14451 | −0.89 | −1.59 | −0.2 | 1.19E−02 | 2.83E−02 |
| Thiamine-triphosphatase | THTPA | Q9BU02 | −0.89 | −1.35 | −0.44 | 1.25E−04 | 5.08E−04 |
| A-kinase anchor protein 7 isoforms alpha and | AKAP7 | O43687 | −0.89 | −1.64 | −0.14 | 1.96E−02 | 4.38E−02 |
| beta | | | | | | | |
| Alpha-(1,3)-fucosyltransferase 10 | FUT10 | Q6P4F1 | −0.89 | −1.38 | −0.4 | 4.11E−04 | 1.42E−03 |
| SLIT and NTRK-like protein 3 | SLITRK3 | O94933 | −0.9 | −1.15 | −0.66 | 1.10E−12 | 3.54E−11 |
| Butyrophilin-like protein 3 | BTNL3 | Q6UXE8 | −0.9 | −1.57 | −0.24 | 7.55E−03 | 1.88E−02 |
| Zinc finger protein 774 | ZNF774 | Q6NX45 | −0.9 | −1.56 | −0.23 | 8.08E−03 | 2.00E−02 |
| Beclin-1 | BECN1 | Q14457 | −0.9 | −1.55 | −0.25 | 6.49E−03 | 1.66E−02 |
| Myeloid zinc finger 1 | MZF1 | P28698 | −0.9 | −1.39 | −0.41 | 3.08E−04 | 1.10E−03 |
| Klotho | KL | Q9UEF7 | −0.9 | −1.25 | −0.55 | 4.99E−07 | 5.02E−06 |
| Paralemmin-1 | PALM | O75781 | −0.9 | −1.62 | −0.17 | 1.55E−02 | 3.56E−02 |
| Complement component C8 | C8A C8B C8G | P07360\|P07358\|P07357 | −0.9 | −1.31 | −0.49 | 1.80E−05 | 9.95E−05 |
| Aurora kinase B | AURKB | Q96GD4 | −0.9 | −1.52 | −0.27 | 5.21E−03 | 1.36E−02 |
| Pregnancy-specific beta-1-glycoprotein 6 | PSG6 | Q00889 | −0.9 | −1.62 | −0.18 | 1.47E−02 | 3.41E−02 |
| Protein CREG1 | CREG1 | O75629 | −0.9 | −1.38 | −0.41 | 2.75E−04 | 1.00E−03 |
| PTB domain-containing engulfment adapter | GULP1 | Q9UBP9 | −0.91 | −1.51 | −0.31 | 2.77E−03 | 7.67E−03 |
| protein 1 | | | | | | | |
| Protein FAM151A | FAM151A | Q8WW52 | −0.91 | −1.23 | −0.59 | 2.22E−08 | 3.05E−07 |
| Transcription factor RelB | RELB | Q01201 | −0.92 | −1.59 | −0.24 | 7.86E−03 | 1.96E−02 |
| Cellular retinoic acid-binding protein 1 | CRABP1 | P29762 | −0.92 | −1.54 | −0.3 | 3.61E−03 | 9.77E−03 |
| ES1 protein homolog, mitochondrial | C21orf33 | P0DPI2 | −0.92 | −1.62 | −0.21 | 1.11E−02 | 2.65E−02 |
| Oxygen-dependent coproporphyrinogen-III | CPOX | P36551 | −0.93 | −1.26 | −0.61 | 2.54E−08 | 3.45E−07 |
| oxidase, mitochondrial | | | | | | | |
| Ficolin-2 | FCN2 | Q15485 | −0.93 | −1.2 | −0.66 | 1.54E−11 | 4.01E−10 |
| Thrombospondin type-1 domain-containing | THSD1 | Q9NS62 | −0.93 | −1.57 | −0.29 | 4.18E−03 | 1.12E−02 |
| protein 1 | | | | | | | |
| Semenogelin-1 | SEMG1 | P04279 | −0.93 | −1.54 | −0.32 | 2.85E−03 | 7.86E−03 |
| Kazal-type serine protease inhibitor domain- | KAZALD1 | Q96I82 | −0.93 | −1.38 | −0.49 | 4.32E−05 | 2.08E−04 |
| containing protein 1 | | | | | | | |
| Keratin, type II cytoskeletal 5 | KRT5 | P13647 | −0.94 | −1.34 | −0.54 | 3.91E−06 | 2.84E−05 |
| E3 ubiquitin-protein ligase NEURL1 | NEURL1 | O76050 | −0.94 | −1.71 | −0.16 | 1.77E−02 | 4.00E−02 |
| Interleukin-17 receptor D | IL17RD | Q8NFM7 | −0.94 | −1.55 | −0.33 | 2.60E−03 | 7.23E−03 |
| Orexigenic neuropeptide QRFP | QRFP | P83859 | −0.94 | −1.56 | −0.32 | 3.04E−03 | 8.37E−03 |
| Insulin gene enhancer protein ISL-1 | ISL1 | P61371 | −0.95 | −1.54 | −0.35 | 1.83E−03 | 5.27E−03 |
| Neuronal pentraxin receptor | NPTXR | O95502 | −0.95 | −1.29 | −0.61 | 5.90E−08 | 7.32E−07 |
| Ubiquitin D | UBD | O15205 | −0.95 | −1.69 | −0.2 | 1.28E−02 | 3.02E−02 |
| Trypsin-3 | PRSS3 | P35030 | −0.95 | −1.25 | −0.64 | 9.98E−10 | 1.88E−08 |
| CD81 antigen | CD81 | P60033 | −0.95 | −1.62 | −0.27 | 5.75E−03 | 1.48E−02 |
| Carboxypeptidase N subunit 2 | CPN2 | P22792 | −0.95 | −1.6 | −0.29 | 4.79E−03 | 1.26E−02 |
| Neogenin | NEO1 | Q92859 | −0.95 | −1.7 | −0.2 | 1.27E−02 | 3.00E−02 |
| Nuclear pore complex-interacting protein family | NPIPB3 | Q92617 | −0.95 | −1.56 | −0.34 | 2.34E−03 | 6.60E−03 |
| member B3 | | | | | | | |
| Fatty acid-binding protein 9 | FABP9 | Q0Z7S8 | −0.96 | −1.37 | −0.56 | 3.28E−06 | 2.43E−05 |
| Seizure 6-like protein | SEZ6L | Q9BYH1 | −0.96 | −1.65 | −0.27 | 6.71E−03 | 1.71E−02 |
| U6 snRNA-associated Sm-like protein LSm4 | LSM4 | Q9Y4Z0 | −0.97 | −1.6 | −0.35 | 2.27E−03 | 6.42E−03 |
| MARCKS-related protein | MARCKSL1 | P49006 | −0.97 | −1.73 | −0.21 | 1.21E−02 | 2.87E−02 |
| Neuromedin-S | NMS | Q5H8A3 | −0.97 | −1.69 | −0.26 | 7.86E−03 | 1.96E−02 |
| Valine--tRNA ligase | VARS | P26640 | −0.98 | −1.52 | −0.44 | 3.94E−04 | 1.36E−03 |
| Retinol-binding protein 4 | RBP4 | P02753 | −0.98 | −1.48 | −0.48 | 1.17E−04 | 4.80E−04 |
| Interleukin-19 | IL19 | Q9UHD0 | −0.98 | −1.57 | −0.39 | 1.19E−03 | 3.62E−03 |
| Neural cell adhesion molecule 2 | NCAM2 | O15394 | −0.98 | −1.41 | −0.55 | 8.61E−06 | 5.46E−05 |
| Interleukin-10 receptor subunit alpha | IL10RA | Q13651 | −0.99 | −1.62 | −0.37 | 1.89E−03 | 5.42E−03 |
| Arfaptin-2 | ARFIP2 | P53365 | −0.99 | −1.35 | −0.63 | 6.27E−08 | 7.71E−07 |
| Interleukin-34 | IL34 | Q6ZMJ4 | −0.99 | −1.4 | −0.58 | 2.01E−06 | 1.62E−05 |
| Dynein intermediate chain 1, axonemal | DNAI1 | Q9UI46 | −1 | −1.74 | −0.25 | 9.20E−03 | 2.25E−02 |
| Carnitine O-palmitoyltransferase 1, muscle | CPT1B | Q92523 | −1 | −1.57 | −0.43 | 5.82E−04 | 1.92E−03 |
| isoform | | | | | | | |
| Phosphoenolpyruvate carboxykinase, cytosolic | PCK1 | P35558 | −1 | −1.47 | −0.53 | 2.97E−05 | 1.51E−04 |
| [GTP] | | | | | | | |
| Guanine nucleotide-binding protein | GNG11 | P61952 | −1 | −1.61 | −0.39 | 1.37E−03 | 4.09E−03 |
| G(I)/G(S)/G(O) subunit gamma-11 | | | | | | | |
| Tyrosine-protein kinase receptor TYRO3 | TYRO3 | Q06418 | −1.01 | −1.57 | −0.45 | 4.09E−04 | 1.41E−03 |
| Prosaposin | PSAP | P07602 | −1.01 | −1.51 | −0.51 | 7.25E−05 | 3.21E−04 |
| Cytochrome P450 3A4 | CYP3A4 | P08684 | −1.01 | −1.68 | −0.34 | 2.99E−03 | 8.24E−03 |
| Histone acetyltransferase type B catalytic subunit | HAT1 | O14929 | −1.02 | −1.41 | −0.64 | 2.05E−07 | 2.32E−06 |
| CD209 antigen | CD209 | Q9NNX6 | −1.02 | −1.39 | −0.65 | 5.14E−08 | 6.51E−07 |
| V-set and transmembrane domain-containing | VSTM4 | Q8IW00 | −1.02 | −1.72 | −0.32 | 4.36E−03 | 1.15E−02 |
| protein 4 | | | | | | | |
| Protein zwilch homolog | ZWILCH | Q9H900 | −1.03 | −1.64 | −0.43 | 8.13E−04 | 2.57E−03 |
| Calcineurin subunit B type 2 | PPP3R2 | Q96LZ3 | −1.03 | −1.56 | −0.51 | 1.24E−04 | 5.07E−04 |
| Bone morphogenetic protein 1 | BMP1 | P13497 | −1.03 | −1.4 | −0.67 | 3.60E−08 | 4.73E−07 |
| Retinoblastoma-associated protein | RB1 | P06400 | −1.03 | −1.7 | −0.36 | 2.72E−03 | 7.54E−03 |
| Tenascin-R | TNR | Q92752 | −1.04 | −1.66 | −0.43 | 8.80E−04 | 2.76E−03 |
| Cyclic AMP-dependent transcription factor ATF- | ATF6B | Q99941 | −1.04 | −1.61 | −0.47 | 3.77E−04 | 1.31E−03 |
| 6 beta | | | | | | | |
| Adenylyltransferase and sulfurtransferase | MOCS3 | O95396 | −1.04 | −1.64 | −0.44 | 6.62E−04 | 2.15E−03 |
| MOCS3 | | | | | | | |
| Signal transducer and activator of transcription | STAT5B | P51692 | −1.04 | −1.81 | −0.27 | 8.17E−03 | 2.03E−02 |
| 5B | | | | | | | |
| Cytochrome c | CYCS | P99999 | −1.04 | −1.49 | −0.59 | 7.16E−06 | 4.70E−05 |
| Glypican-3 | GPC3 | P51654 | −1.04 | −1.42 | −0.66 | 1.05E−07 | 1.25E−06 |
| Guanylate-binding protein 6 | GBP6 | Q6ZN66 | −1.04 | −1.59 | −0.49 | 2.27E−04 | 8.52E−04 |
| Ribosomal protein S6 kinase beta-1 | RPS6KB1 | P23443 | −1.04 | −1.58 | −0.5 | 1.67E−04 | 6.54E−04 |
| Protein shisa-2 homolog | SHISA2 | Q6UWI4 | −1.05 | −1.68 | −0.42 | 1.06E−03 | 3.26E−03 |
| Semaphorin-4B | SEMA4B | Q9NPR2 | −1.05 | −1.56 | −0.53 | 7.38E−05 | 3.26E−04 |
| Ras-related protein Rab-17 | RAB17 | Q9H0T7 | −1.06 | −1.58 | −0.53 | 9.46E−05 | 4.02E−04 |
| Cytochrome b561 domain-containing protein 1 | CYB561D1 | Q8N8Q1 | −1.06 | −1.57 | −0.55 | 5.13E−05 | 2.38E−04 |
| Protein S100-A10 | S100A10 | P60903 | −1.06 | −1.74 | −0.39 | 2.00E−03 | 5.72E−03 |
| Prion-like protein doppel | PRND | Q9UKY0 | −1.06 | −1.6 | −0.51 | 1.43E−04 | 5.69E−04 |
| Chromobox protein homolog 5 | CBX5 | P45973 | −1.06 | −1.64 | −0.47 | 4.01E−04 | 1.39E−03 |
| Ceruloplasmin | CP | P00450 | −1.07 | −1.59 | −0.54 | 7.06E−05 | 3.13E−04 |
| L-Selectin | SELL | P14151 | −1.07 | −1.71 | −0.44 | 9.77E−04 | 3.04E−03 |
| CMP-N-acetylneuraminate-beta-galactosamide- | ST3GAL1 | Q11201 | −1.07 | −1.48 | −0.65 | 4.82E−07 | 4.92E−06 |
| alpha-2,3-sialyltransferase 1 | | | | | | | |
| Carboxypeptidase M | CPM | P14384 | −1.07 | −1.63 | −0.51 | 1.81E−04 | 6.97E−04 |
| Protocadherin beta-2 | PCDHB2 | Q9Y5E7 | −1.08 | −1.56 | −0.6 | 1.15E−05 | 6.91E−05 |
| Polypeptide N-acetylgalactosaminyltransferase | GALNT13 | Q8IUC8 | −1.08 | −1.5 | −0.65 | 6.68E−07 | 6.41E−06 |
| 13 | | | | | | | |
| Hexokinase-1 | HK1 | P19367 | −1.08 | −1.69 | −0.46 | 5.65E−04 | 1.87E−03 |
| Harmonin | USH1C | Q9Y6N9 | −1.08 | −1.85 | −0.31 | 6.10E−03 | 1.56E−02 |
| Transcription initiation factor TFIID subunit 10 | TAF10 | Q12962 | −1.08 | −1.56 | −0.6 | 9.90E−06 | 6.12E−05 |
| WAP, Kazal, immunoglobulin, Kunitz and NTR | WFIKKN2 | Q8TEU8 | −1.08 | −1.58 | −0.58 | 2.54E−05 | 1.32E−04 |
| domain-containing protein 2 | | | | | | | |
| Alpha-1,3-mannosyl-glycoprotein 4-beta-N- | MGAT4B | Q9UQ53 | −1.08 | −1.56 | −0.59 | 1.45E−05 | 8.28E−05 |
| acetylglucosaminyltransferase B | | | | | | | |
| Protein sel-1 homolog 2 | SEL1L2 | Q5TEA6 | −1.08 | −1.77 | −0.38 | 2.38E−03 | 6.70E−03 |
| Guanylyl cyclase-activating protein 1 | GUCA1A | P43080 | −1.09 | −1.7 | −0.48 | 4.31E−04 | 1.48E−03 |
| Regulator of G-protein signaling 8 | RGS8 | P57771 | −1.09 | −1.68 | −0.5 | 2.78E−04 | 1.01E−03 |
| Fibroblast growth factor receptor 3: Extracellular | FGFR3 | P22607 | −1.09 | −1.47 | −0.7 | 3.48E−08 | 4.61E−07 |
| domain | | | | | | | |
| Retinoic acid early transcript 1G protein | RAET1G | Q6H3X3 | −1.09 | −1.67 | −0.51 | 2.27E−04 | 8.52E−04 |
| Tyrosine-protein phosphatase non-receptor type 4 | PTPN4 | P29074 | −1.1 | −1.69 | −0.51 | 2.84E−04 | 1.03E−03 |
| Vitamin K-dependent protein S | PROS1 | P07225 | −1.1 | −1.88 | −0.32 | 5.45E−03 | 1.42E−02 |
| Transmembrane protein 132B | TMEM132B | Q14DG7 | −1.1 | −1.63 | −0.57 | 5.15E−05 | 2.38E−04 |
| C-type lectin domain family 10 member A | CLEC10A | Q8IUN9 | −1.11 | −1.7 | −0.51 | 2.73E−04 | 9.98E−04 |
| Short-chain specific acyl-CoA dehydrogenase, | ACADS | P16219 | −1.11 | −1.78 | −0.44 | 1.12E−03 | 3.44E−03 |
| mitochondrial | | | | | | | |
| Surfeit locus protein 1 | SURF1 | Q15526 | −1.12 | −1.53 | −0.71 | 7.38E−08 | 8.98E−07 |
| Glypican-1 | GPC1 | P35052 | −1.12 | −1.63 | −0.6 | 2.02E−05 | 1.09E−04 |
| Beta-1,4-galactosyltransferase 6 | B4GALT6 | Q9UBX8 | −1.13 | −1.61 | −0.65 | 3.71E−06 | 2.72E−05 |
| Ras-related protein Rab-35 | RAB35 | Q15286 | −1.13 | −1.73 | −0.54 | 2.04E−04 | 7.76E−04 |
| Ephrin-A3 | EFNA3 | P52797 | −1.13 | −1.68 | −0.58 | 5.29E−05 | 2.44E−04 |
| Toll-like receptor 1: Extracellular domain | TLR1 | Q15399 | −1.13 | −1.91 | −0.36 | 4.24E−03 | 1.13E−02 |
| GDNF family receptor alpha-2 | GFRA2 | O00451 | −1.13 | −1.86 | −0.39 | 2.66E−03 | 7.40E−03 |
| Uronyl 2-sulfotransferase | UST | Q9Y2C2 | −1.13 | −1.69 | −0.58 | 7.03E−05 | 3.12E−04 |
| EMILIN-3: region 1 | EMILIN3 | Q9NT22 | −1.13 | −1.57 | −0.7 | 2.72E−07 | 3.00E−06 |
| Transmembrane protein 132C: Extracellular | TMEM132C | Q8N3T6 | −1.14 | −1.55 | −0.74 | 2.85E−08 | 3.84E−07 |
| domain | | | | | | | |
| Appetite-regulating hormone | GHRL | Q9UBU3 | −1.14 | −1.73 | −0.55 | 1.51E−04 | 5.97E−04 |
| Phosphatidylinositol transfer protein beta isoform | PITPNB | P48739 | −1.15 | −1.86 | −0.44 | 1.60E−03 | 4.69E−03 |
| SH2 domain-containing protein 3C | SH2D3C | Q8N5H7 | −1.15 | −1.86 | −0.43 | 1.66E−03 | 4.85E−03 |
| Ficolin-2 | FCN2 | Q15485 | −1.15 | −1.56 | −0.73 | 7.19E−08 | 8.78E−07 |
| Apolipoprotein A-I | APOA1 | P02647 | −1.15 | −1.71 | −0.59 | 6.18E−05 | 2.79E−04 |
| Coagulation factor Xa | F10 | P00742 | −1.15 | −1.72 | −0.57 | 8.74E−05 | 3.76E−04 |
| Insulin-like growth factor 1 receptor | IGF1R | P08069 | −1.15 | −1.67 | −0.64 | 1.27E−05 | 7.43E−05 |
| Sperm-associated antigen 11A | SPAG11A | Q6PDA7 | −1.15 | −1.7 | −0.6 | 3.95E−05 | 1.92E−04 |
| C1GALT1-specific chaperone 1 | C1GALT1C1 | Q96EU7 | −1.15 | −1.88 | −0.41 | 2.21E−03 | 6.27E−03 |
| Uncharacterized protein C11orf87 | C11orf87 | Q6NUJ2 | −1.16 | −1.95 | −0.36 | 4.38E−03 | 1.16E−02 |
| Glutathione S-transferase A4 | GSTA4 | O15217 | −1.16 | −1.68 | −0.64 | 1.16E−05 | 6.94E−05 |
| RGM domain family member B | RGMB | Q6NW40 | −1.16 | −1.91 | −0.41 | 2.39E−03 | 6.74E−03 |
| C-type lectin domain family 4 member D | CLEC4D | Q8WXI8 | −1.16 | −1.89 | −0.43 | 1.76E−03 | 5.09E−03 |
| Leukocyte immunoglobulin-like receptor | LILRA4 | P59901 | −1.16 | −1.63 | −0.7 | 9.08E−07 | 8.16E−06 |
| subfamily A member 4 | | | | | | | |
| C-X-C motif chemokine 9 | CXCL9 | Q07325 | −1.17 | −2.05 | −0.29 | 8.89E−03 | 2.18E−02 |
| Iduronate 2-sulfatase | IDS | P22304 | −1.17 | −1.67 | −0.67 | 5.18E−06 | 3.60E−05 |
| Angiostatin | PLG | P00747 | −1.17 | −1.96 | −0.38 | 3.85E−03 | 1.04E−02 |
| Uncharacterized protein C1orf185 | C1orf185 | Q5T7R7 | −1.18 | −1.88 | −0.48 | 1.01E−03 | 3.12E−03 |
| rRNA 2′-O-methyltransferase fibrillarin | FBL | P22087 | −1.18 | −1.89 | −0.46 | 1.29E−03 | 3.89E−03 |
| Interleukin-1 Receptor accessory protein | IL1RAP | Q9NPH3 | −1.18 | −1.59 | −0.78 | 7.97E−09 | 1.24E−07 |
| Complement component 1 Q subcomponent- | C1QBP | Q07021 | −1.18 | −2.04 | −0.32 | 6.96E−03 | 1.76E−02 |
| binding protein, mitochondrial | | | | | | | |
| Neuroplastin | NPTN | Q9Y639 | −1.18 | −1.62 | −0.73 | 2.57E−07 | 2.86E−06 |
| Cadherin-7 | CDH7 | Q9ULB5 | −1.18 | −1.67 | −0.7 | 1.69E−06 | 1.39E−05 |
| Secretogranin-3 | SCG3 | Q8WXD2 | −1.19 | −1.72 | −0.66 | 1.04E−05 | 6.37E−05 |
| Serine protease 57 | PRSS57 | Q6UWY2 | −1.19 | −1.66 | −0.72 | 6.53E−07 | 6.32E−06 |
| Interleukin-6 receptor subunit beta | IL6ST | P40189 | −1.2 | −2.09 | −0.32 | 7.89E−03 | 1.96E−02 |
| Urotensin-2 | UTS2 | O95399 | −1.2 | −1.78 | −0.62 | 5.01E−05 | 2.33E−04 |
| Vesicular, overexpressed in cancer, prosurvival | VOPP1 | Q96AW1 | −1.21 | −1.68 | −0.74 | 4.13E−07 | 4.30E−06 |
| protein 1 | | | | | | | |
| Reticulocalbin-3 | RCN3 | Q96D15 | −1.21 | −1.79 | −0.63 | 4.51E−05 | 2.15E−04 |
| Complement C1r subcomponent | C1R | P00736 | −1.21 | −1.64 | −0.77 | 5.85E−08 | 7.30E−07 |
| Tumor necrosis factor-inducible gene 6 protein | TNFAIP6 | P98066 | −1.22 | −1.62 | −0.81 | 4.76E−09 | 7.87E−08 |
| IgLON family member 5 | IGLON5 | A6NGN9 | −1.22 | −1.67 | −0.77 | 1.03E−07 | 1.22E−06 |
| Glycosaminoglycan xylosylkinase | FAM20B | O75063 | −1.22 | −2.04 | −0.4 | 3.70E−03 | 9.99E−03 |
| Galanin | GAL | P22466 | −1.23 | −2 | −0.45 | 2.01E−03 | 5.73E−03 |
| Coagulation factor VII | F7 | P08709 | −1.23 | −1.72 | −0.74 | 7.91E−07 | 7.28E−06 |
| Coagulation Factor X | F10 | P00742 | −1.23 | −1.81 | −0.65 | 3.37E−05 | 1.67E−04 |
| Leukemia inhibitory factor receptor | LIFR | P42702 | −1.23 | −1.73 | −0.73 | 1.21E−06 | 1.05E−05 |
| SLIT and NTRK-like protein 4 | SLITRK4 | Q8IW52 | −1.23 | −1.68 | −0.79 | 6.13E−08 | 7.57E−07 |
| Sodium channel subunit beta-2 | SCN2B | O60939 | −1.23 | −1.67 | −0.8 | 2.16E−08 | 2.97E−07 |
| Carbonic anhydrase 6 | CA6 | P23280 | −1.24 | −1.46 | −1.02 | 1.23E−27 | 3.40E−25 |
| Cathepsin L2 | CTSV | O60911 | −1.24 | −1.6 | −0.87 | 5.31E−11 | 1.26E−09 |
| Serine/threonine-protein kinase PAK 3 | PAK3 | O75914 | −1.24 | −1.75 | −0.73 | 2.33E−06 | 1.85E−05 |
| Thrombin | F2 | P00734 | −1.24 | −1.6 | −0.88 | 1.41E−11 | 3.71E−10 |
| Collectin-10 | COLEC10 | Q9Y6Z7 | −1.24 | −2.01 | −0.48 | 1.47E−03 | 4.33E−03 |
| Sclerostin | SOST | Q9BQB4 | −1.25 | −1.88 | −0.62 | 9.37E−05 | 3.99E−04 |
| Small conductance calcium-activated potassium | KCNN1 | Q92952 | −1.25 | −2.02 | −0.48 | 1.43E−03 | 4.23E−03 |
| channel protein 1 | | | | | | | |
| Liver carboxylesterase 1 | CES1 | P23141 | −1.25 | −1.78 | −0.72 | 3.71E−06 | 2.72E−05 |
| Syndecan-3 | SDC3 | O75056 | −1.25 | −1.77 | −0.73 | 3.09E−06 | 2.31E−05 |
| Receptor tyrosine-protein kinase erbB-3 | ERBB3 | P21860 | −1.25 | −1.71 | −0.79 | 1.15E−07 | 1.35E−06 |
| Ectonucleoside triphosphate diphosphohydrolase | ENTPD1 | P49961 | −1.25 | −1.91 | −0.59 | 2.18E−04 | 8.22E−04 |
| 1 | | | | | | | |
| Inositol monophosphatase 3 | IMPAD1 | Q9NX62 | −1.26 | −1.71 | −0.8 | 5.54E−08 | 6.95E−07 |
| Alanine aminotransferase 1 | GPT | P24298 | −1.27 | −1.81 | −0.73 | 4.67E−06 | 3.29E−05 |
| Aggrecan core protein | ACAN | P16112 | −1.27 | −1.82 | −0.73 | 4.42E−06 | 3.14E−05 |
| Dual specificity mitogen-activated protein kinase | MAP2K4 | P45985 | −1.27 | −1.65 | −0.89 | 4.35E−11 | 1.06E−09 |
| kinase 4 | | | | | | | |
| Testican-2 | SPOCK2 | Q92563 | −1.27 | −1.71 | −0.83 | 1.37E−08 | 2.01E−07 |
| Inositol polyphosphate 5-phosphatase OCRL-1 | OCRL | Q01968 | −1.28 | −1.83 | −0.72 | 6.31E−06 | 4.23E−05 |
| Dipeptidase 1 | DPEP1 | P16444 | −1.28 | −1.7 | −0.87 | 1.79E−09 | 3.20E−08 |
| Melanocyte protein PMEL | PMEL | P40967 | −1.29 | −1.66 | −0.93 | 3.17E−12 | 9.28E−11 |
| Amyloid-like protein 1 | APLP1 | P51693 | −1.29 | −1.62 | −0.95 | 6.30E−14 | 2.40E−12 |
| Mast/stem cell growth factor receptor Kit | KIT | P10721 | −1.3 | −1.73 | −0.86 | 5.33E−09 | 8.69E−08 |
| Protein MENT | MENT | Q9BUN1 | −1.31 | −1.71 | −0.91 | 1.26E−10 | 2.81E−09 |
| Potassium voltage-gated channel subfamily E | KCNE5 | Q9UJ90 | −1.31 | −1.97 | −0.64 | 1.18E−04 | 4.84E−04 |
| regulatory beta subunit 5:Cytoplasmic domain | | | | | | | |
| Desmoglein-2 | DSG2 | Q14126 | −1.31 | −1.89 | −0.73 | 9.56E−06 | 5.96E−05 |
| Myosin regulatory light chain 2, atrial isoform | MYL7 | Q01449 | −1.32 | −1.85 | −0.78 | 1.27E−06 | 1.09E−05 |
| BDNF/NT-3 growth factors receptor | NTRK2 | Q16620 | −1.32 | −1.75 | −0.88 | 3.19E−09 | 5.42E−08 |
| Coagulation factor XIII B chain | F13B | P05160 | −1.32 | −1.85 | −0.78 | 1.56E−06 | 1.30E−05 |
| Antibacterial protein LL-37 | CAMP | P49913 | −1.33 | −1.77 | −0.89 | 3.13E−09 | 5.34E−08 |
| Iron-sulfur cluster co-chaperone protein HscB, | HSCB | Q8IWL3 | −1.33 | −1.92 | −0.73 | 1.18E−05 | 6.99E−05 |
| mitochondrial | | | | | | | |
| UPF0577 protein KIAA1324: Extracellular | KIAA1324 | Q6UXG2 | −1.34 | −1.87 | −0.81 | 7.99E−07 | 7.33E−06 |
| domain | | | | | | | |
| Phosphatidylinositol 4-phosphate 3-kinase C2 | PIK3C2A | O00443 | −1.34 | −1.95 | −0.72 | 1.89E−05 | 1.04E−04 |
| domain-containing subunit alpha | | | | | | | |
| Indian hedgehog protein | IHH | Q14623 | −1.34 | −1.8 | −0.89 | 9.28E−09 | 1.41E−07 |
| Beta-defensin 1 | DEFB1 | P60022 | −1.34 | −1.77 | −0.91 | 8.96E−10 | 1.71E−08 |
| Type 2 lactosamine alpha-2,3-sialyltransferase | ST3GAL6 | Q9Y274 | −1.34 | −1.79 | −0.89 | 8.15E−09 | 1.26E−07 |
| Endothelin-converting enzyme 1: Cytoplasmic | ECE1 | P42892 | −1.34 | −1.99 | −0.69 | 6.12E−05 | 2.77E−04 |
| domain | | | | | | | |
| Multidrug resistance-associated protein 6 | ABCC6 | O95255 | −1.34 | −2.05 | −0.63 | 2.15E−04 | 8.13E−04 |
| C-type lectin domain family 2 member A | CLEC2A | Q6UVW9 | −1.35 | −1.94 | −0.75 | 8.75E−06 | 5.54E−05 |
| Scavenger receptor class A member 3: region 2 | SCARA3 | Q6AZY7 | −1.35 | −2.11 | −0.6 | 4.57E−04 | 1.56E−03 |
| Integral membrane protein 2B | ITM2B | Q9Y287 | −1.35 | −1.93 | −0.76 | 5.93E−06 | 4.01E−05 |
| Cyclin-dependent kinase 4 inhibitor C | CDKN2C | P42773 | −1.36 | −1.99 | −0.74 | 1.64E−05 | 9.19E−05 |
| 14-3-3 protein epsilon | YWHAE | P62258 | −1.36 | −1.94 | −0.78 | 4.67E−06 | 3.29E−05 |
| Noncompact myelin-associated protein | NCMAP | Q5T1S8 | −1.36 | −1.8 | −0.92 | 1.37E−09 | 2.49E−08 |
| Bone morphogenetic protein 8B | BMP8B | P34820 | −1.36 | −2.04 | −0.69 | 6.97E−05 | 3.10E−04 |
| Peflin | PEF1 | Q9UBV8 | −1.36 | −1.9 | −0.82 | 6.73E−07 | 6.44E−06 |
| Cytochrome c oxidase subunit 6C | COX6C | P09669 | −1.36 | −1.96 | −0.76 | 9.89E−06 | 6.12E−05 |
| G2/mitotic-specific cyclin-B2 | CCNB2 | O95067 | −1.37 | −2.09 | −0.65 | 2.05E−04 | 7.78E−04 |
| Delta and Notch-like epidermal growth factor- | DNER | Q8NFT8 | −1.37 | −1.77 | −0.97 | 2.58E−11 | 6.52E−10 |
| related receptor: Extracellular domain | | | | | | | |
| Signal-transducing adaptor protein 1 | STAP1 | Q9ULZ2 | −1.38 | −2.18 | −0.58 | 7.60E−04 | 2.42E−03 |
| Kininogen, HMW, Two Chain | KNG1 | P01042 | −1.38 | −1.79 | −0.97 | 3.22E−11 | 7.97E−10 |
| Proteasome subunit alpha type-5 | PSMA5 | P28066 | −1.39 | −1.83 | −0.94 | 1.55E−09 | 2.80E−08 |
| Prolyl endopeptidase FAP | FAP | Q12884 | −1.39 | −2.02 | −0.76 | 1.58E−05 | 8.92E−05 |
| Stromelysin-1 | MMP3 | P08254 | −1.4 | −1.96 | −0.84 | 8.68E−07 | 7.90E−06 |
| Baculoviral IAP repeat-containing protein 5 | BIRC5 | O15392 | −1.41 | −2.22 | −0.6 | 6.76E−04 | 2.19E−03 |
| Beta-defensin 104 | DEFB104A | Q8WTQ1 | −1.41 | −1.75 | −1.06 | 2.03E−15 | 9.61E−14 |
| Sulfhydryl oxidase 2 | QSOX2 | Q6ZRP7 | −1.41 | −1.9 | −0.91 | 2.47E−08 | 3.38E−07 |
| tRNA (guanine-N(7)-)-methyltransferase | METTL1 | Q9UBP6 | −1.42 | −2.16 | −0.69 | 1.45E−04 | 5.76E−04 |
| TOM1-like protein 1 | TOM1L1 | O75674 | −1.42 | −2.07 | −0.77 | 2.00E−05 | 1.08E−04 |
| Rab9 effector protein with kelch motifs | RABEPK | Q7Z6M1 | −1.43 | −2.17 | −0.7 | 1.20E−04 | 4.91E−04 |
| Cystatin-M | CST6 | Q15828 | −1.44 | −1.83 | −1.06 | 2.97E−13 | 1.03E−11 |
| Interleukin-6 receptor subunit alpha | IL6R | P08887 | −1.45 | −1.95 | −0.95 | 1.71E−08 | 2.43E−07 |
| Prenylcysteine oxidase 1 | PCYOX1 | Q9UHG3 | −1.45 | −1.94 | −0.97 | 5.34E−09 | 8.69E−08 |
| Protein kinase C-binding protein NELL1 | NELL1 | Q92832 | −1.45 | −1.77 | −1.13 | 9.25E−19 | 6.23E−17 |
| Receptor-type tyrosine-protein phosphatase delta | PTPRD | P23468 | −1.45 | −1.87 | −1.03 | 1.49E−11 | 3.91E−10 |
| Carbonic anhydrase-related protein 10 | CA10 | Q9NS85 | −1.46 | −1.91 | −1.01 | 2.21E−10 | 4.69E−09 |
| Proprotein convertase subtilisin/kexin type 7 | PCSK7 | Q16549 | −1.46 | −1.95 | −0.97 | 5.59E−09 | 9.04E−08 |
| Histone-lysine N-methyltransferase EHMT2 | EHMT2 | Q96KQ7 | −1.47 | −1.89 | −1.05 | 8.35E−12 | 2.30E−10 |
| Proactivator polypeptide-like 1 | PSAPL1 | Q6NUJ1 | −1.48 | −1.86 | −1.1 | 3.43E−14 | 1.38E−12 |
| Cytochrome c oxidase subunit 4 isoform 2, | COX4I2 | Q96KJ9 | −1.49 | −2.1 | −0.87 | 2.36E−06 | 1.86E−05 |
| mitochondrial | | | | | | | |
| Golgi-associated plant pathogenesis-related | GLIPR2 | Q9H4G4 | −1.5 | −2.1 | −0.9 | 9.00E−07 | 8.12E−06 |
| protein 1 | | | | | | | |
| Neurexophilin-3 | NXPH3 | O95157 | −1.5 | −2.24 | −0.75 | 8.67E−05 | 3.74E−04 |
| Chymotrypsin-like elastase family member 2A | CELA2A | P08217 | −1.5 | −1.82 | −1.17 | 3.24E−19 | 2.48E−17 |
| Platelet endothelial aggregation receptor | PEAR1 | Q5VY43 | −1.5 | −2.25 | −0.75 | 9.14E−05 | 3.90E−04 |
| 1: Extracellular domain | | | | | | | |
| Neurocan core protein | NCAN | O14594 | −1.52 | −1.87 | −1.17 | 1.68E−17 | 1.01E−15 |
| Heme oxygenase 1 | HMOX1 | P09601 | −1.52 | −2 | −1.05 | 3.19E−10 | 6.50E−09 |
| von Willebrand factor A domain-containing | VWA2 | Q5GFL6 | −1.52 | −1.96 | −1.08 | 1.14E−11 | 3.06E−10 |
| protein 2 | | | | | | | |
| Calcium-activated potassium channel subunit | KCNMB3 | Q9NPA1 | −1.52 | −2.19 | −0.85 | 8.96E−06 | 5.61E−05 |
| beta-3 | | | | | | | |
| Ecto-ADP-ribosyltransferase 3 | ART3 | Q13508 | −1.55 | −1.98 | −1.12 | 1.76E−12 | 5.52E−11 |
| Cartilage intermediate layer protein 2 | CILP2 | Q8IUL8 | −1.55 | −1.93 | −1.17 | 1.63E−15 | 8.02E−14 |
| Beta-1,4-galactosyltransferase 2 | B4GALT2 | O60909 | −1.55 | −2.43 | −0.66 | 5.94E−04 | 1.95E−03 |
| Hematopoietic prostaglandin D synthase | HPGDS | O60760 | −1.57 | −1.97 | −1.16 | 3.56E−14 | 1.42E−12 |
| Leucine-rich repeat and fibronectin type III | LRFN1 | Q9P244 | −1.57 | −2.24 | −0.9 | 4.14E−06 | 2.98E−05 |
| domain-containing protein 1 | | | | | | | |
| Insulin-like growth factor-binding protein | IGFALS | P35858 | −1.58 | −2.01 | −1.15 | 4.58E−13 | 1.54E−11 |
| complex acid labile subunit | | | | | | | |
| Ephrin type-A receptor 4 | EPHA4 | P54764 | −1.59 | −2.37 | −0.82 | 6.12E−05 | 2.77E−04 |
| Potassium voltage-gated channel subfamily G | KCNG4 | Q8TDN1 | −1.61 | −2.29 | −0.93 | 3.53E−06 | 2.59E−05 |
| member 4 | | | | | | | |
| Alpha-1-antichymotrypsin | SERPINA3 | P01011 | −1.62 | −2.96 | −0.29 | 1.74E−02 | 3.95E−02 |
| Transmembrane protein 132A | TMEM132A | Q24JP5 | −1.62 | −2.14 | −1.11 | 6.80E−10 | 1.33E−08 |
| T-complex protein 11 homolog | TCP11 | Q8WWU5 | −1.62 | −2.26 | −0.98 | 6.15E−07 | 5.98E−06 |
| Solute carrier family 22 member 16 | SLC22A16 | Q86VW1 | −1.62 | −2.15 | −1.1 | 1.45E−09 | 2.62E−08 |
| Dixin | DIXDC1 | Q155Q3 | −1.63 | −2.43 | −0.83 | 6.70E−05 | 3.00E−04 |
| Heat shock 70 kDa protein 1A | HSPA1A | P0DMV8 | −1.64 | −2.37 | −0.91 | 9.69E−06 | 6.02E−05 |
| T-cell surface glycoprotein CD3 epsilon chain | CD3E | P07766 | −1.65 | −2.17 | −1.13 | 6.36E−10 | 1.26E−08 |
| Transmembrane protein 9: Cytoplasmic domain | TMEM9 | Q9POT7 | −1.66 | −2.38 | −0.94 | 5.96E−06 | 4.03E−05 |
| Endothelial monocyte-activating polypeptide 2 | AIMP1 | Q12904 | −1.67 | −2.35 | −0.98 | 1.75E−06 | 1.44E−05 |
| Hyaluronidase-1 | HYAL1 | Q12794 | −1.7 | −2.45 | −0.95 | 8.50E−06 | 5.42E−05 |
| Sodium-coupled monocarboxylate transporter 1 | SLC5A8 | Q8N695 | −1.71 | −2.17 | −1.25 | 4.18E−13 | 1.44E−11 |
| Fibroblast growth factor 9 | FGF9 | P31371 | −1.71 | −2.33 | −1.08 | 9.44E−08 | 1.13E−06 |
| Apolipoprotein B | APOB | P04114 | −1.71 | −2.18 | −1.25 | 7.25E−13 | 2.39E−11 |
| Ficolin-3 | FCN3 | O75636 | −1.71 | −2.2 | −1.23 | 5.29E−12 | 1.50E−10 |
| Uncharacterized protein C3orf18 | C3orf18 | Q9UK00 | −1.71 | −2.57 | −0.84 | 1.04E−04 | 4.39E−04 |
| E3 ubiquitin-protein ligase RNF146 | RNF146 | Q9NTX7 | −1.74 | −2.54 | −0.95 | 1.80E−05 | 9.98E−05 |
| Prostaglandin F2 receptor negative regulator | PTGFRN | Q9P2B2 | −1.74 | −2.19 | −1.29 | 5.37E−14 | 2.09E−12 |
| Muellerian-inhibiting factor | AMH | P03971 | −1.75 | −2.43 | −1.06 | 5.31E−07 | 5.25E−06 |
| Inter-alpha-trypsin inhibitor heavy chain H1 | ITIH1 | P19827 | −1.78 | −2.53 | −1.02 | 4.30E−06 | 3.08E−05 |
| Palmitoleoyl-protein carboxylesterase NOTUM | NOTUM | Q6P988 | −1.78 | −2.17 | −1.39 | 7.04E−19 | 4.94E−17 |
| Growth/differentiation factor 2 | GDF2 | Q9UK05 | −1.79 | −2.49 | −1.1 | 4.92E−07 | 4.99E−06 |
| Torsin-1 A-interacting protein 1: Nuclear domain | TOR1AIP1 | Q5JTV8 | −1.81 | −2.36 | −1.27 | 7.17E−11 | 1.65E−09 |
| NT-3 growth factor receptor | NTRK3 | Q16288 | −1.83 | −2.34 | −1.33 | 1.28E−12 | 4.06E−11 |
| Anthrax toxin receptor 1 | ANTXR1 | Q9H6X2 | −1.86 | −2.36 | −1.37 | 1.75E−13 | 6.36E−12 |
| F-box/LRR-repeat protein 4: Leucine-rich repeats | FBXL4 | Q9UKA2 | −1.87 | −2.77 | −0.98 | 4.01E−05 | 1.95E−04 |
| 4 and 5 | | | | | | | |
| Cadherin-3 | CDH3 | P22223 | −1.87 | −2.46 | −1.27 | 9.24E−10 | 1.76E−08 |
| Platelet-activating factor acetylhydrolase | PLA2G7 | Q13093 | −1.87 | −2.28 | −1.46 | 8.55E−19 | 5.83E−17 |
| Ectonucleotide | ENPP5 | Q9UJA9 | −1.87 | −2.3 | −1.44 | 2.98E−17 | 1.74E−15 |
| pyrophosphatase/phosphodiesterase family | | | | | | | |
| member 5 | | | | | | | |
| Interferon alpha-2 | IFNA2 | P01563 | −1.93 | −2.81 | −1.06 | 1.53E−05 | 8.69E−05 |
| Ectonucleoside triphosphate diphosphohydrolase | ENTPD5 | O75356 | −1.95 | −2.57 | −1.32 | 1.14E−09 | 2.10E−08 |
| 5 | | | | | | | |
| Cyclic AMP-dependent transcription factor ATF- | ATF6 | P18850 | −1.99 | −2.6 | −1.38 | 1.58E−10 | 3.44E−09 |
| 6 alpha | | | | | | | |
| Complement C1q tumor necrosis factor-related | C1QTNF3 | Q9BXJ4 | −2.04 | −2.69 | −1.39 | 7.63E−10 | 1.48E−08 |
| protein 3 | | | | | | | |
| Interleukin-1 receptor type 2 | IL1R2 | P27930 | −2.08 | −2.66 | −1.5 | 2.00E−12 | 6.14E−11 |
| Afamin | AFM | P43652 | −2.09 | −2.83 | −1.36 | 2.11E−08 | 2.92E−07 |
| Calsequestrin-1 | CASQ1 | P31415 | −2.1 | −3.03 | −1.17 | 9.53E−06 | 5.94E−05 |
| Tumor necrosis factor ligand superfamily | TNFSF12 | O43508 | −2.1 | −2.68 | −1.52 | 1.12E−12 | 3.59E−11 |
| member 12 | | | | | | | |
| Anthrax toxin receptor 2 | ANTXR2 | P58335 | −2.12 | −2.56 | −1.68 | 3.83E−21 | 3.74E−19 |
| Kidney-associated antigen 1 | KAAG1 | Q9UBP8 | −2.14 | −2.71 | −1.58 | 1.25E−13 | 4.62E−12 |
| N-acetylglucosamine-1-phosphodiester alpha-N- | NAGPA | Q9UK23 | −2.15 | −2.61 | −1.68 | 3.52E−19 | 2.66E−17 |
| acetylglucosaminidase | | | | | | | |
| Cholinesterase | BCHE | P06276 | −2.15 | −2.75 | −1.56 | 2.12E−12 | 6.49E−11 |
| Mannosyl-oligosaccharide 1,2-alpha- | MAN1A2 | O60476 | −2.15 | −3.18 | −1.13 | 4.07E−05 | 1.97E−04 |
| mannosidase IB | | | | | | | |
| Hemopexin | HPX | P02790 | −2.17 | −3.36 | −0.99 | 3.39E−04 | 1.20E−03 |
| Cell adhesion molecule-related/down-regulated | CDON | Q4KMG0 | −2.17 | −2.81 | −1.53 | 3.65E−11 | 9.00E−10 |
| by oncogenes | | | | | | | |
| Tartrate-resistant acid phosphatase type 5 | ACP5 | P13686 | −2.19 | −2.82 | −1.57 | 6.47E−12 | 1.82E−10 |
| Glucoside xylosyltransferase 1 | GXYLT1 | Q4G148 | −2.19 | −2.74 | −1.64 | 6.92E−15 | 3.02E−13 |
| Zinc-alpha-2-glycoprotein | AZGP1 | P25311 | −2.2 | −2.81 | −1.59 | 2.43E−12 | 7.32E−11 |
| Semaphorin-3G | SEMA3G | Q9NS98 | −2.21 | −2.89 | −1.54 | 1.23E−10 | 2.75E−09 |
| Voltage-dependent calcium channel subunit | CACNA2D3 | Q8IZS8 | −2.21 | −2.68 | −1.74 | 2.80E−20 | 2.45E−18 |
| alpha-2/delta-3 | | | | | | | |
| Ciliary neurotrophic factor receptor subunit alpha | CNTFR | P26992 | −2.25 | −2.9 | −1.6 | 1.05E−11 | 2.84E−10 |
| Leucine-rich repeats and immunoglobulin-like | LRIG3 | Q6UXM1 | −2.25 | −2.76 | −1.75 | 1.81E−18 | 1.19E−16 |
| domains protein 3 | | | | | | | |
| Carboxypeptidase B2 | CPB2 | Q96IY4 | −2.25 | −3.23 | −1.27 | 7.44E−06 | 4.85E−05 |
| Hedgehog-interacting protein | HHIP | Q96QV1 | −2.26 | −2.96 | −1.57 | 2.00E−10 | 4.26E−09 |
| Sodium/potassium-transporting ATPase subunit | ATP1B1 | P05026 | −2.26 | −2.64 | −1.87 | 1.59E−30 | 7.22E−28 |
| beta-1 | | | | | | | |
| Carbonic anhydrase 4 | CA4 | P22748 | −2.3 | −2.99 | −1.62 | 4.55E−11 | 1.09E−09 |
| Plasma kallikrein | KLKB1 | P03952 | −2.3 | −3.08 | −1.53 | 6.28E−09 | 9.95E−08 |
| Gelsolin | GSN | P06396 | −2.32 | −3.33 | −1.32 | 6.30E−06 | 4.22E−05 |
| Neuronal growth regulator 1 | NEGR1 | Q7Z3B1 | −2.34 | −3.2 | −1.48 | 9.43E−08 | 1.13E−06 |
| Contactin-1 | CNTN1 | Q12860 | −2.34 | −3.04 | −1.64 | 7.48E−11 | 1.71E−09 |
| Serotransferrin | TF | P02787 | −2.34 | −3.63 | −1.05 | 3.90E−04 | 1.36E−03 |
| N-acetyllactosaminide beta-1,3-N- | B3GNT2 | Q9NY97 | −2.35 | −3.25 | −1.44 | 4.06E−07 | 4.26E−06 |
| acetylglucosaminyltransferase 2 | | | | | | | |
| N-acetylmuramoyl-L-alanine amidase | PGLYRP2 | Q96PD5 | −2.36 | −3.2 | −1.52 | 3.50E−08 | 4.61E−07 |
| A disintegrin and metalloproteinase with | ADAMTS13 | Q76LX8 | −2.4 | −2.9 | −1.9 | 4.18E−21 | 4.00E−19 |
| thrombospondin motifs 13 | | | | | | | |
| Contactin-4 | CNTN4 | Q8IWV2 | −2.46 | −3.15 | −1.76 | 5.14E−12 | 1.46E−10 |
| Complement component C8 gamma chain | C8G | P07360 | −2.48 | −3.28 | −1.68 | 1.13E−09 | 2.10E−08 |
| Neuronal pentraxin-2 | NPTX2 | P47972 | −2.52 | −3.22 | −1.82 | 1.83E−12 | 5.70E−11 |
| Repulsive guidance molecule A | RGMA | Q96B86 | −2.55 | −3.44 | −1.67 | 1.48E−08 | 2.16E−07 |
| E3 ubiquitin-protein ligase DTX1 | DTX1 | Q86Y01 | −2.59 | −3.34 | −1.83 | 1.94E−11 | 4.95E−10 |
| Zinc finger protein 334 | ZNF334 | Q9HCZ1 | −2.59 | −3.3 | −1.88 | 1.03E−12 | 3.32E−11 |
| Biotinidase | BTD | P43251 | −2.59 | −3.31 | −1.86 | 3.32E−12 | 9.66E−11 |
| Superoxide dismutase [Mn], mitochondrial | SOD2 | P04179 | −2.63 | −3.16 | −2.1 | 3.87E−22 | 4.38E−20 |
| Group XIIB secretory phospholipase A2-like | PLA2G12B | Q9BX93 | −2.64 | −3.13 | −2.15 | 5.97E−26 | 1.24E−23 |
| protein | | | | | | | |
| Matrix-remodeling-associated protein | MXRA8 | Q9BRK3 | −2.85 | −3.43 | −2.27 | 1.70E−21 | 1.73E−19 |
| 8: Extracellular domain | | | | | | | |
| Vascular endothelial growth factor receptor 2 | KDR | P35968 | −2.85 | −3.56 | −2.14 | 4.20E−15 | 1.92E−13 |
| Prothrombin | F2 | P00734 | −2.87 | −3.97 | −1.76 | 3.63E−07 | 3.89E−06 |
| Alpha-2-HS-glycoprotein | AHSG | P02765 | −2.93 | −3.78 | −2.07 | 2.30E−11 | 5.83E−10 |
| S-methyl-5′-thioadenosine phosphorylase | MTAP | Q13126 | −2.93 | −3.69 | −2.17 | 4.05E−14 | 1.60E−12 |
| Collagen alpha-1(XIII) chain | COL13A1 | Q5TAT6 | −2.95 | −3.56 | −2.35 | 1.80E−21 | 1.80E−19 |
| Immunoglobulin superfamily DCC subclass | IGDCC4 | Q8TDY8 | −2.95 | −3.52 | −2.37 | 1.47E−23 | 1.88E−21 |
| member 4 | | | | | | | |
| Coiled-coil domain-containing protein 126 | CCDC126 | Q96EE4 | −2.97 | −3.5 | −2.44 | 6.42E−28 | 1.88E−25 |
| Hepatocyte growth factor receptor | MET | P08581 | −2.99 | −3.65 | −2.33 | 8.22E−19 | 5.69E−17 |
| Protein SET | SET | Q01105 | −2.99 | −3.86 | −2.13 | 1.10E−11 | 2.96E−10 |
| Tetranectin | CLEC3B | P05452 | −3.19 | −4.06 | −2.32 | 7.66E−13 | 2.51E−11 |
| Receptor-type tyrosine-protein phosphatase eta | PTPRJ | Q12913 | −3.31 | −4.02 | −2.6 | 8.10E−20 | 6.61E−18 |
| Alpha-2-antiplasmin | SERPINF2 | P08697 | −3.4 | −4.48 | −2.31 | 9.81E−10 | 1.85E−08 |
| EF-hand domain-containing protein D1 | EFHD1 | Q9BUP0 | −3.41 | −4.37 | −2.46 | 2.60E−12 | 7.79E−11 |
| Neural cell adhesion molecule 1, 120 kDa | NCAM1 | P13591 | −3.45 | −4.09 | −2.81 | 6.84E−26 | 1.36E−23 |
| isoform | | | | | | | |
| Vitamin D-binding protein | GC | P02774 | −3.62 | −4.57 | −2.66 | 1.23E−13 | 4.56E−12 |
| Apolipoprotein M | APOM | O95445 | −3.68 | −4.22 | −3.14 | 9.78E−40 | 1.62E−36 |
| Inter-alpha-trypsin inhibitor heavy chain H2 | ITIH2 | P19823 | −3.68 | −4.63 | −2.74 | 2.98E−14 | 1.21E−12 |
| B melanoma antigen 3 | BAGE3 | Q86Y29 | −3.74 | −4.52 | −2.96 | 1.01E−20 | 9.34E−19 |
| Receptor-type tyrosine-protein phosphatase S | PTPRS | Q13332 | −3.8 | −4.61 | −2.99 | 4.69E−20 | 4.02E−18 |
| Heparan-sulfate 6-O-sulfotransferase 3 | HS6ST3 | Q8IZP7 | −3.81 | −4.67 | −2.96 | 3.95E−18 | 2.52E−16 |
| Vitamin K-dependent protein C | PROC | P04070 | −3.85 | −4.56 | −3.13 | 1.04E−25 | 2.00E−23 |
| Kallistatin | SERPINA4 | P29622 | −3.98 | −4.98 | −2.98 | 6.98E−15 | 3.02E−13 |
| Neuropeptide S | NPS | P0C0P6 | −4.43 | −5.26 | −3.61 | 1.35E−25 | 2.50E−23 |
| Serum albumin | ALB | P02768 | −4.8 | −5.82 | −3.79 | 2.63E−20 | 2.38E−18 |
| Apolipoprotein C-I | APOC1 | P02654 | −4.85 | −5.79 | −3.91 | 1.21E−23 | 1.63E−21 |
| Antithrombin-III | SERPINC1 | P01008 | −5.06 | −6.48 | −3.65 | 2.90E−12 | 8.56E−11 |
| Bifunctional heparan sulfate N-deacetylase/N- | NDST1 | P52848 | −5.25 | −6.25 | −4.25 | 1.34E−24 | 2.09E−22 |
| sulfotransferase 1 | | | | | | | |
| Neurogenic locus notch homolog protein 1 | NOTCH1 | P46531 | −5.42 | −6.61 | −4.23 | 6.14E−19 | 4.37E−17 |
| Epidermal growth factor receptor | EGFR | P00533 | −6.35 | −7.2 | −5.51 | 1.19E−47 | 5.90E−44 |

In the CARDIA cohort, there were 1,003 proteins (961 GeneIDs) significant for QIA, with 575 proteins positively and 428 negatively associated with QIA (Table 3).

TABLE 3
Univariate regression analysis of QIA in CARDIA

| | | | Per- | | | | |
|---|---|---|---|---|---|---|---|
| | | | cent | Up- | Low- | | |
| | | | QIA | per | er | | |
| | Entrez | | per | 95% | 95% | p- | FDR p- |
| Protein name | gene name | UniProt ID | ln(RFU) | CI | CI | value | value |
| Complement factor H | CFH | P08603 | 9.65 | 7.35 | 11.96 | 2.95E−16 | 3.68E−14 |
| Complement factor B | CFB | P00751 | 6.05 | 4.69 | 7.4 | 3.47E−18 | 7.50E−16 |
| Coagulation factor IXab | F9 | P00740 | 5.1 | 3.86 | 6.34 | 9.12E−16 | 1.01E−13 |
| Rho-related GTP-binding protein RhoG | RHOG | P84095 | 5.02 | 2.88 | 7.17 | 4.48E−06 | 5.99E−05 |
| Pigment epithelium-derived factor | SERPINF1 | P36955 | 5.02 | 3.47 | 6.56 | 2.21E−10 | 7.15E−09 |
| Insulin-like growth factor-binding protein 4 | IGFBP4 | P22692 | 4.62 | 3.72 | 5.53 | 3.05E−23 | 3.04E−20 |
| Complement factor I | CFI | P05156 | 4.59 | 3.1 | 6.09 | 1.86E−09 | 5.12E−08 |
| Fibrinogen gamma chain | FGG | P02679 | 4.49 | 3.16 | 5.82 | 4.04E−11 | 1.53E−09 |
| Ubiquitin-conjugating enzyme E2 G2 | UBE2G2 | P60604 | 4.3 | 3.31 | 5.28 | 1.85E−17 | 3.42E−15 |
| Gamma-aminobutyric acid receptor-associated | GABARAP | O95166 | 4.28 | 3.28 | 5.27 | 6.46E−17 | 1.03E−14 |
| protein | | | | | | | |
| ADP-ribosylation factor-like protein 1 | ARL1 | P40616 | 4.18 | 2.9 | 5.45 | 1.55E−10 | 5.30E−09 |
| Serine protease HTRA1 | HTRA1 | Q92743 | 4.13 | 3.27 | 4.98 | 6.80E−21 | 2.60E−18 |
| D-dimer | FGA\|FGB\|FGG | P02671\|P02675\|P02679 | 4.07 | 2.8 | 5.34 | 3.37E−10 | 1.04E−08 |
| Rho GTPase-activating protein 36 | ARHGAP36 | Q6ZRI8 | 4.01 | 3.18 | 4.85 | 8.03E−21 | 2.86E−18 |
| Alpha-1 -microglobulin | AMBP | P02760 | 4 | 2.68 | 5.32 | 3.49E−09 | 8.83E−08 |
| Collagen alpha-1(XXVIII) chain | COL28A1 | Q2UY09 | 3.92 | 3.07 | 4.78 | 4.33E−19 | 1.27E−16 |
| Heat shock 70 kDa protein 1A | HSPA1A | P0DMV8 | 3.92 | 2.27 | 5.57 | 3.43E−06 | 4.72E−05 |
| Matrix Gla protein | MGP | P08493 | 3.86 | 2.82 | 4.89 | 3.17E−13 | 2.08E−11 |
| Retinoic acid receptor responder protein 2 | RARRES2 | Q99969 | 3.85 | 3.08 | 4.62 | 2.55E−22 | 1.82E−19 |
| Sialic acid-binding Ig-like lectin I2: Ig-like | SIGLEC12 | Q96PQ1 | 3.77 | 2.73 | 4.81 | 1.69E−12 | 8.57E−11 |
| C2-type 2 domain, Isoform short | | | | | | | |
| Coagulation factor IX | F9 | P00740 | 3.75 | 2.65 | 4.85 | 2.73E−11 | 1.11E−09 |
| Collagen alpha-3(VI) chain: Bovine pancreatic | COL6A3 | P12111 | 3.72 | 2.87 | 4.56 | 1.08E−17 | 2.07E−15 |
| trypsin inhibitor/Kunitz inhibitor | | | | | | | |
| domain, isoform 1 | | | | | | | |
| Gamma-aminobutyric acid receptor-associated | GABARAPL1 | Q9H0R8 | 3.69 | 2.73 | 4.66 | 7.00E−14 | 5.28E−12 |
| protein-like 1 | | | | | | | |
| C-X-C motif chemokine 16 | CXCL16 | Q9H2A7 | 3.63 | 2.65 | 4.6 | 3.54E−13 | 2.29E−11 |
| Embryonal Fyn-associated substrate | EFS | O43281 | 3.61 | 2.54 | 4.68 | 3.91E−11 | 1.50E−09 |
| Complement C2 | C2 | P06681 | 3.55 | 2.55 | 4.55 | 3.47E−12 | 1.69E−10 |
| C4b-binding protein alpha chain | C4BPA | P04003 | 3.55 | 2.58 | 4.52 | 8.93E−13 | 4.82E−11 |
| Serum amyloid P-component | APCS | P02743 | 3.5 | 2.52 | 4.48 | 3.27E−12 | 1.63E−10 |
| Lumican | LUM | P51884 | 3.41 | 2.37 | 4.46 | 1.87E−10 | 6.16E−09 |
| EGF-containing fibulin-like extracellular matrix | EFEMP1 | Q12805 | 3.41 | 2.45 | 4.37 | 4.25E−12 | 2.03E−10 |
| protein 1 | | | | | | | |
| Leukocyte immunoglobulin-like receptor | LILRA5 | A6NI73 | 3.37 | 2.59 | 4.15 | 4.14E−17 | 6.87E−15 |
| subfamily A member 5 | | | | | | | |
| Gamma-glutamyl hydrolase | GGH | Q92820 | 3.29 | 2.55 | 4.03 | 5.58E−18 | 1.16E−15 |
| Serine/arginine-rich splicing factor 7 | SRSF7 | Q16629 | 3.27 | 2.39 | 4.15 | 4.76E−13 | 3.00E−11 |
| Fibulin-5 | FBLN5 | Q9UBX5 | 3.23 | 2.1 | 4.35 | 2.05E−08 | 4.51E−07 |
| Peptidyl-prolyl cis-trans isomerase C | PPIC | P45877 | 3.19 | 2.29 | 4.08 | 3.39E−12 | 1.67E−10 |
| Neurofascin | NFASC | O94856 | 3.18 | 2.08 | 4.28 | 1.65E−08 | 3.71E−07 |
| Guanine nucleotide exchange factor VAV3 | VAV3 | Q9UKW4 | 3.17 | 2.13 | 4.21 | 2.34E−09 | 6.29E−08 |
| Interleukin enhancer-binding factor 3 | ILF3 | Q12906 | 3.13 | 2.27 | 3.99 | 1.57E−12 | 8.05E−11 |
| Heat shock 70 kDa protein 1A | HSPA1A | P0DMV8 | 3.09 | 1.96 | 4.22 | 8.61E−08 | 1.71E−06 |
| Dermatopontin | DPT | Q07507 | 3.09 | 2.27 | 3.92 | 2.65E−13 | 1.81E−11 |
| DnaJ homolog subfamily B member 9 | DNAJB9 | Q9UBS3 | 3.08 | 2.36 | 3.8 | 1.02E−16 | 1.46E−14 |
| B melanoma antigen 2 | BAGE2 | Q86Y30 | 3.07 | 2.27 | 3.87 | 7.15E−14 | 5.31E−12 |
| Tumor necrosis factor ligand superfamily | TNFSF15 | O95150 | 3.06 | 2.06 | 4.06 | 2.41E−09 | 6.43E−08 |
| member 15 | | | | | | | |
| RNA-binding protein EWS | EWSR1 | Q01844 | 3.03 | 2.06 | 4 | 9.94E−10 | 2.88E−08 |
| Pyridoxine-5′-phosphate oxidase | PNPO | Q9NVS9 | 3 | 1.81 | 4.18 | 7.66E−07 | 1.22E−05 |
| DCN1-like protein 1 | DCUN1D1 | Q96GG9 | 2.99 | 2.12 | 3.86 | 1.99E−11 | 8.49E−10 |
| HLA class I histocompatibility antigen, alpha | HLA-G | P17693 | 2.97 | 1.34 | 4.59 | 3.62E−04 | 2.97E−03 |
| chain G | | | | | | | |
| Serine/arginine-rich splicing factor 6 | SRSF6 | Q13247 | 2.96 | 2.23 | 3.69 | 2.79E−15 | 2.62E−13 |
| Out at first protein homolog | OAF | Q86UD1 | 2.95 | 1.98 | 3.93 | 3.25E−09 | 8.30E−08 |
| Proteasome subunit beta type-3 | PSMB3 | P49720 | 2.91 | 1.87 | 3.96 | 4.59E−08 | 9.60E−07 |
| Coiled-coil domain-containing protein 80 | CCDC80 | Q76M96 | 2.91 | 1.84 | 3.98 | 1.07E−07 | 2.07E−06 |
| Coagulation factor XIII | F13A1\|F13B | P00488\|P05160 | 2.88 | 1.64 | 4.12 | 5.64E−06 | 7.37E−05 |
| Anion exchange transporter | SLC26A7 | Q8TE54 | 2.87 | 1.67 | 4.06 | 2.57E−06 | 3.64E−05 |
| Prenylcysteine oxidase-like | PCYOX1L | Q8NBM8 | 2.87 | 1.59 | 4.14 | 1.03E−05 | 1.27E−04 |
| Cystatin-C | CST3 | P01034 | 2.86 | 1.93 | 3.78 | 1.37E−09 | 3.91E−08 |
| Ganglioside GM2 activator | GM2A | P17900 | 2.84 | 2.01 | 3.68 | 3.18E−11 | 1.28E−09 |
| Furin | FURIN | P09958 | 2.83 | 1.73 | 3.94 | 5.45E−07 | 8.98E−06 |
| Leucine-rich repeats and immunoglobulin-like | LRIG1 | Q96JA1 | 2.81 | 1.94 | 3.67 | 2.06E−10 | 6.69E−09 |
| domains protein 1 | | | | | | | |
| Inhibin beta C chain | INHBC | P55103 | 2.79 | 2.08 | 3.49 | 1.71E−14 | 1.45E−12 |
| WD repeat-containing protein 5 | WDR5 | P61964 | 2.78 | 1.75 | 3.81 | 1.40E−07 | 2.65E−06 |
| Low-density lipoprotein receptor-related | LRP11 | Q86VZ4 | 2.7 | 1.77 | 3.63 | 1.41E−08 | 3.21E−07 |
| protein 11 | | | | | | | |
| Trafficking protein particle complex subunit 3 | TRAPPC3 | O43617 | 2.69 | 1.96 | 3.42 | 6.87E−13 | 3.84E−11 |
| Ribonuclease K6 | RNASE6 | Q93091 | 2.69 | 2.03 | 3.35 | 2.33E−15 | 2.32E−13 |
| Heat shock 70 kDa protein 1A | HSPA1A | P0DMV8 | 2.69 | 1.75 | 3.62 | 1.76E−08 | 3.95E−07 |
| Complement C1q tumor necrosis factor-related | C1QTNF1 | Q9BXJ1 | 2.67 | 1.47 | 3.87 | 1.25E−05 | 1.53E−04 |
| protein 1 | | | | | | | |
| ADAMTS-like protein 2 | ADAMTSL2 | Q86TH1 | 2.66 | 1.89 | 3.43 | 1.28E−11 | 5.67E−10 |
| Rho GTPase-activating protein 30 | ARHGAP30 | Q7Z616 | 2.64 | 1.51 | 3.76 | 4.68E−06 | 6.24E−05 |
| Histone-binding protein RBBP4 | RBBP4 | Q09028 | 2.62 | 1.67 | 3.57 | 7.66E−08 | 1.54E−06 |
| 5-hydroxytryptamine receptor 6 | HTR6 | P50406 | 2.59 | 1.75 | 3.43 | 1.67E−09 | 4.68E−08 |
| Complement C3 | C3 | P01024 | 2.59 | 1.77 | 3.41 | 5.94E−10 | 1.77E−08 |
| Thrombospondin-4 | THBS4 | P35443 | 2.55 | 1.73 | 3.37 | 1.25E−09 | 3.58E−08 |
| Vesicular integral-membrane protein VIP36 | LMAN2 | Q12907 | 2.55 | 1.81 | 3.3 | 2.41E−11 | 1.00E−09 |
| Reticulon-4 receptor | RTN4R | Q9BZR6 | 2.54 | 1.75 | 3.34 | 3.85E−10 | 1.18E−08 |
| Protein phosphatase 1 regulatory subunit 1A | PPP1R1A | Q13522 | 2.53 | 1.68 | 3.38 | 6.95E−09 | 1.69E−07 |
| Beta-2-microglobulin | B2M | P61769 | 2.53 | 1.84 | 3.23 | 1.33E−12 | 6.91E−11 |
| Plastin-2 | LCP1 | P13796 | 2.52 | 1.31 | 3.73 | 4.63E−05 | 4.86E−04 |
| Heat shock cognate 71 kDa protein | HSPA8 | P11142 | 2.52 | 1.67 | 3.37 | 5.93E−09 | 1.46E−07 |
| C-C motif chemokine 18 | CCL18 | P55774 | 2.49 | 1.88 | 3.1 | 1.85E−15 | 1.88E−13 |
| C-C motif chemokine 22 | CCL22 | O00626 | 2.45 | 1.83 | 3.06 | 7.54E−15 | 6.71E−13 |
| Protein ABHD14A | ABHD14A | Q9BUJ0 | 2.45 | 1.74 | 3.16 | 1.31E−11 | 5.77E−10 |
| Nucleobindin-1 | NUCB1 | Q02818 | 2.42 | 1.44 | 3.39 | 1.21E−06 | 1.84E−05 |
| Oncoprotein-induced transcript 3 protein | OIT3 | Q8WWZ8 | 2.42 | 1.69 | 3.14 | 6.66E−11 | 2.44E−09 |
| Putative hydrolase RBBP9 | RBBP9 | O75884 | 2.41 | 0.65 | 4.18 | 7.36E−03 | 3.84E−02 |
| Complement C4 | C4A\|C4B | P0C0L4\|P0C0L5 | 2.41 | 1.27 | 3.54 | 3.26E−05 | 3.61E−04 |
| Malignant T-cell-amplified sequence 1 | MCTS1 | Q9ULC4 | 2.4 | 1.84 | 2.96 | 6.61E−17 | 1.03E−14 |
| Interleukin-1 receptor antagonist protein | IL1RN | P18510 | 2.4 | 1.83 | 2.96 | 1.30E−16 | 1.80E−14 |
| Lamina-associated polypeptide 2, isoforms | TMPO | P42167 | 2.37 | 1.73 | 3.01 | 4.83E−13 | 3.00E−11 |
| beta/gamma | | | | | | | |
| Microfibrillar-associated protein 2 | MFAP2 | P55001 | 2.37 | 1.51 | 3.24 | 8.40E−08 | 1.68E−06 |
| Folate receptor beta | FOLR2 | P14207 | 2.36 | 1.48 | 3.24 | 1.54E−07 | 2.90E−06 |
| Galectin-9 | LGALS9 | O00182 | 2.36 | 1.44 | 3.28 | 5.29E−07 | 8.78E−06 |
| Switch-associated protein 70 | SWAP70 | Q9UH65 | 2.34 | 1.29 | 3.4 | 1.26E−05 | 1.53E−04 |
| Insulin | INS | P01308 | 2.33 | 1.73 | 2.93 | 4.10E−14 | 3.19E−12 |
| C-C motif chemokine 21 | CCL21 | O00585 | 2.32 | 1.63 | 3.01 | 3.87E−11 | 1.50E−09 |
| Serine/threonine-protein phosphatase 4 | PPP4R3A | Q6IN85 | 2.31 | 1.66 | 2.96 | 3.87E−12 | 1.87E−10 |
| regulatory subunit 3A | | | | | | | |
| Follistatin-related protein 3 | FSTL3 | O95633 | 2.31 | 1.46 | 3.17 | 1.15E−07 | 2.20E−06 |
| Testican-1 | SPOCK1 | Q08629 | 2.29 | 0.89 | 3.69 | 1.39E−03 | 9.41E−03 |
| Transformer-2 protein homolog beta | TRA2B | P62995 | 2.28 | 1.66 | 2.9 | 6.60E−13 | 3.78E−11 |
| Procollagen-lysine, 2-oxoglutarate 5- | PLOD3 | O60568 | 2.27 | 1.4 | 3.14 | 3.46E−07 | 6.04E−06 |
| dioxygenase 3 | | | | | | | |
| Heat shock 70 kDa protein 1B | HSPA1B | P0DMV9 | 2.27 | 1.43 | 3.11 | 1.27E−07 | 2.41E−06 |
| Thrombospondin-3 | THBS3 | P49746 | 2.27 | 1.58 | 2.95 | 8.73E−11 | 3.10E−09 |
| Zinc finger protein 75D | ZNF75D | P51815 | 2.26 | 1.51 | 3.01 | 4.06E−09 | 1.02E−07 |
| Ribonuclease 4 | RNASE4 | P34096 | 2.25 | 1.05 | 3.45 | 2.45E−04 | 2.08E−03 |
| WNT1-inducible-signaling pathway protein 2 | CCN5 | O76076 | 2.23 | 1.4 | 3.07 | 1.67E−07 | 3.09E−06 |
| Suppressor of cytokine signaling 3 | SOCS3 | O14543 | 2.22 | 1.42 | 3.02 | 5.84E−08 | 1.19E−06 |
| Calsyntenin-3 | CLSTN3 | Q9BQT9 | 2.22 | 1.62 | 2.83 | 6.35E−13 | 3.68E−11 |
| Complement C1s subcomponent | C1S | P09871 | 2.22 | 0.73 | 3.71 | 3.55E−03 | 2.09E−02 |
| Protein SEC13 homolog | SEC13 | P55735 | 2.21 | 0.82 | 3.59 | 1.76E−03 | 1.15E−02 |
| Dynactin subunit 2 | DCTN2 | Q13561 | 2.21 | 0.93 | 3.48 | 7.07E−04 | 5.35E−03 |
| Urotensin-2B | UTS2B | Q765I0 | 2.21 | 1.23 | 3.18 | 8.78E−06 | 1.10E−04 |
| Extracellular sulfatase Sulf-2 | SULF2 | Q8IWU5 | 2.2 | 1.3 | 3.1 | 1.89E−06 | 2.79E−05 |
| ADP-ribosylation factor-like protein 15 | ARL15 | Q9NXU5 | 2.18 | 1.44 | 2.92 | 9.24E−09 | 2.20E−07 |
| GDNF family receptor alpha-1 | GFRA1 | P56159 | 2.18 | 1.39 | 2.96 | 5.79E−08 | 1.19E−06 |
| Tumor necrosis factor receptor superfamily | TNFRSF1B | P20333 | 2.18 | 1.5 | 2.86 | 3.32E−10 | 1.03E−08 |
| member 1B | | | | | | | |
| Nidogen-1 | NID1 | P14543 | 2.17 | 1.27 | 3.06 | 2.35E−06 | 3.38E−05 |
| Heterogeneous nuclear ribonucleoproteins | HNRNPA2B1 | P22626 | 2.16 | 1.45 | 2.87 | 2.81E−09 | 7.32E−08 |
| A2/B1 | | | | | | | |
| Complement C3d fragment | C3 | P01024 | 2.15 | 1.52 | 2.78 | 2.04E−11 | 8.62E−10 |
| Tolloid-like protein 1 | TLL1 | O43897 | 2.15 | 1.22 | 3.08 | 5.76E−06 | 7.50E−05 |
| Serum amyloid A-4 protein | SAA4 | P35542 | 2.14 | 1.6 | 2.68 | 6.82E−15 | 6.18E−13 |
| Acyl-CoA-binding domain-containing protein 6 | ACBD6 | Q9BR61 | 2.14 | 1.14 | 3.15 | 3.07E−05 | 3.45E−04 |
| Nuclease-sensitive element-binding protein 1 | YBX1 | P67809 | 2.14 | 1.48 | 2.79 | 1.53E−10 | 5.27E−09 |
| Macrophage-capping protein | CAPG | P40121 | 2.13 | 1.56 | 2.7 | 2.70E−13 | 1.82E−11 |
| Kynureninase | KYNU | Q16719 | 2.12 | 1.17 | 3.06 | 1.11E−05 | 1.37E−04 |
| Serpin B8 | SERPINB8 | P50452 | 2.1 | 1.23 | 2.97 | 2.45E−06 | 3.50E−05 |
| Asialoglycoprotein receptor 1 | ASGR1 | P07306 | 2.1 | 1.33 | 2.87 | 8.99E−08 | 1.78E−06 |
| Glycolipid transfer protein domain-containing | GLTPD2 | A6NH11 | 2.1 | 1.3 | 2.91 | 3.16E−07 | 5.56E−06 |
| protein 2 | | | | | | | |
| Histone deacetylase complex subunit SAP18 | SAP18 | O00422 | 2.09 | 1.26 | 2.93 | 8.29E−07 | 1.30E−05 |
| Plexin-D1 | PLXND1 | Q9Y4D7 | 2.09 | 1.16 | 3.02 | 1.15E−05 | 1.41E−04 |
| Fatty acid-binding protein, heart | FABP3 | P05413 | 2.08 | 1.68 | 2.47 | 7.40E−25 | 9.21E−22 |
| Uncharacterized protein C14orf93 | C14orf93 | Q9H972 | 2.08 | 1.02 | 3.13 | 1.21E−04 | 1.14E−03 |
| Golgi membrane protein 1 | GOLM1 | Q8NBJ4 | 2.05 | 1.47 | 2.62 | 2.54E−12 | 1.28E−10 |
| Vascular endothelial growth factor A | VEGFA | P15692 | 2.04 | 0.72 | 3.36 | 2.52E−03 | 1.56E−02 |
| Peroxidasin homolog | PXDN | Q92626 | 2.03 | 1.53 | 2.53 | 3.19E−15 | 2.94E−13 |
| von Willebrand factor A domain-containing | VWA1 | Q6PCB0 | 2.01 | 0.91 | 3.11 | 3.36E−04 | 2.77E−03 |
| protein 1 | | | | | | | |
| Protein-tyrosine sulfotransferase 2 | TPST2 | O60704 | 2.01 | 0.48 | 3.53 | 9.91E−03 | 4.96E−02 |
| Fatty acid-binding protein, adipocyte | FABP4 | P15090 | 2 | 1.59 | 2.4 | 1.17E−21 | 7.13E−19 |
| Tumor necrosis factor receptor superfamily | TNFRSF1A | P19438 | 2 | 1.24 | 2.77 | 3.29E−07 | 5.77E−06 |
| member 1A | | | | | | | |
| Lipopolysaccharide-binding protein | LBP | P18428 | 2 | 1.46 | 2.53 | 2.42E−13 | 1.67E−11 |
| T-cell immunoglobulin and mucin domain- | TIMD4 | Q96H15 | 1.99 | 1.38 | 2.6 | 1.87E−10 | 6.16E−09 |
| containing protein 4 | | | | | | | |
| Protein kinase C zeta type | PRKCZ | Q05513 | 1.99 | 1.09 | 2.89 | 1.51E−05 | 1.79E−04 |
| Plexin-B2 | PLXNB2 | O15031 | 1.99 | 0.85 | 3.13 | 6.28E−04 | 4.83E−03 |
| DnaJ homolog subfamily C member 11 | DNAJC11 | Q9NVH1 | 1.98 | 0.77 | 3.19 | 1.30E−03 | 8.92E−03 |
| Testis-expressed sequence 29 protein | TEX29 | Q8N6K0 | 1.97 | 1.05 | 2.88 | 2.72E−05 | 3.08E−04 |
| Agrin | AGRN | O00468 | 1.96 | 1.13 | 2.8 | 4.28E−06 | 5.80E−05 |
| Deoxyribonuclease-1-like 2 | DNASE1L2 | Q92874 | 1.96 | 1.2 | 2.71 | 3.98E−07 | 6.77E−06 |
| DNA-3-methyladenine glycosylase | MPG | P29372 | 1.95 | 1.14 | 2.76 | 2.56E−06 | 3.63E−05 |
| CUB domain-containing protein 1 | CDCP1 | Q9H5V8 | 1.95 | 1.47 | 2.43 | 2.60E−15 | 2.49E−13 |
| Hepatitis A virus cellular receptor 2 | HAVCR2 | Q8TDQ0 | 1.95 | 1.17 | 2.73 | 1.10E−06 | 1.69E−05 |
| Prefoldin subunit 2 | PFDN2 | Q9UHV9 | 1.94 | 0.91 | 2.97 | 2.17E−04 | 1.88E−03 |
| Stem cell growth factor-beta | CLEC11A | Q9Y240 | 1.91 | 1.25 | 2.57 | 1.79E−08 | 3.99E−07 |
| Colipase-like protein 1 | CLPSL1 | A2RUU4 | 1.89 | 1.24 | 2.53 | 8.87E−09 | 2.14E−07 |
| Hepatocyte nuclear factor 4-alpha | HNF4A | P41235 | 1.87 | 0.98 | 2.76 | 3.81E−05 | 4.09E−04 |
| Ribonuclease pancreatic | RNASE1 | P07998 | 1.87 | 1.46 | 2.28 | 6.87E−19 | 1.90E−16 |
| C-C motif chemokine 3 | CCL3 | P10147 | 1.86 | 1.32 | 2.39 | 1.05E−11 | 4.80E−10 |
| Protein S100-A9 | S100A9 | P06702 | 1.86 | 1.25 | 2.47 | 2.88E−09 | 7.39E−08 |
| Poly(U)-binding-splicing factor PUF60 | PUF60 | Q9UHX1 | 1.85 | 0.79 | 2.9 | 6.01E−04 | 4.66E−03 |
| Sushi, von Willebrand factor type A, EGF and | SVEP1 | Q4LDE5 | 1.85 | 1.18 | 2.53 | 7.73E−08 | 1.55E−06 |
| pentraxin domain-containing protein 1: EGF- | | | | | | | |
| like domains 4-6 | | | | | | | |
| ADP-ribosylation factor-like protein 5B | ARL5B | Q96KC2 | 1.85 | 1.01 | 2.7 | 1.86E−05 | 2.17E−04 |
| Prefoldin subunit 1 | PFDN1 | O60925 | 1.85 | 1.11 | 2.6 | 1.14E−06 | 1.75E−05 |
| Clusterin | CLU | P10909 | 1.85 | 0.52 | 3.19 | 6.46E−03 | 3.46E−02 |
| Ephrin-A4 | EFNA4 | P52798 | 1.84 | 0.81 | 2.87 | 4.58E−04 | 3.63E−03 |
| Transforming growth factor beta-3 | TGFB3 | P10600 | 1.84 | 0.7 | 2.97 | 1.56E−03 | 1.03E−02 |
| Dual specificity protein phosphatase 6 | DUSP6 | Q16828 | 1.83 | 0.81 | 2.86 | 4.63E−04 | 3.66E−03 |
| Tumor necrosis factor ligand superfamily | TNFSF11 | O14788 | 1.83 | 1.01 | 2.66 | 1.29E−05 | 1.56E−04 |
| member 11 | | | | | | | |
| Calcium-binding and coiled-coil domain- | CALCOCO2 | Q13137 | 1.82 | 0.59 | 3.06 | 3.88E−03 | 2.25E−02 |
| containing protein 2 | | | | | | | |
| Ephrin-A2 | EFNA2 | O43921 | 1.82 | 0.89 | 2.75 | 1.33E−04 | 1.23E−03 |
| 60S ribosomal protein L12 | RPL12 | P30050 | 1.82 | 1.25 | 2.4 | 5.75E−10 | 1.72E−08 |
| Ran-binding protein 3 | RANBP3 | Q9H6Z4 | 1.81 | 0.98 | 2.64 | 1.99E−05 | 2.30E−04 |
| Marginal zone B- and B1-cell-specific protein | MZB1 | Q8WU39 | 1.81 | 1.26 | 2.36 | 1.22E−10 | 4.27E−09 |
| Tumor necrosis factor receptor superfamily | TNFRSF1B | P20333 | 1.81 | 1.08 | 2.55 | 1.38E−06 | 2.07E−05 |
| member 1B | | | | | | | |
| Delta-like protein 1 | DLL1 | O00548 | 1.81 | 0.89 | 2.73 | 1.10E−04 | 1.05E−03 |
| Signal-regulatory protein beta-2 | SIRPB2 | Q5JXA9 | 1.81 | 1 | 2.62 | 1.25E−05 | 1.53E−04 |
| Pseudokinase FAM20A | FAM20A | Q96MK3 | 1.81 | 0.83 | 2.8 | 3.06E−04 | 2.55E−03 |
| Glutathione S-transferase omega-1 | GSTO1 | P78417 | 1.8 | 0.7 | 2.9 | 1.36E−03 | 9.24E−03 |
| Copper transport protein ATOX1 | ATOX1 | O00244 | 1.8 | 1.21 | 2.39 | 2.38E−09 | 6.38E−08 |
| Legumain | LGMN | Q99538 | 1.8 | 0.64 | 2.96 | 2.28E−03 | 1.44E−02 |
| Interleukin-18 receptor 1 | IL18R1 | Q13478 | 1.79 | 1.11 | 2.48 | 3.01E−07 | 5.34E−06 |
| Dual specificity mitogen-activated protein | MAP2K2 | P36507 | 1.79 | 1.14 | 2.44 | 7.23E−08 | 1.46E−06 |
| kinase kinase 2 | | | | | | | |
| Kininogen-1 | KNG1 | P01042 | 1.79 | 0.79 | 2.79 | 4.64E−04 | 3.67E−03 |
| Phospholipase A2, membrane associated | PLA2G2A | P14555 | 1.78 | 1.3 | 2.27 | 7.31E−13 | 4.00E−11 |
| Stem cell growth factor-alpha | CLEC11A | Q9Y240 | 1.78 | 1.17 | 2.39 | 1.18E−08 | 2.73E−07 |
| CREB-binding protein | CREBBP | Q92793 | 1.77 | 1.03 | 2.51 | 3.06E−06 | 4.26E−05 |
| Protein FAM49B | CYRIB | Q9NUQ9 | 1.77 | 1.09 | 2.45 | 3.51E−07 | 6.08E−06 |
| Intercellular adhesion molecule 1 | ICAM1 | P05362 | 1.77 | 1.22 | 2.32 | 3.50E−10 | 1.08E−08 |
| E-selectin | SELE | P16581 | 1.76 | 1.24 | 2.28 | 3.47E−11 | 1.37E−09 |
| Leukocyte immunoglobulin-like receptor | LILRA5 | A6NI73 | 1.76 | 0.97 | 2.55 | 1.45E−05 | 1.73E−04 |
| subfamily A member 5 | | | | | | | |
| Integrin beta-2 | ITGB2 | P05107 | 1.75 | 0.83 | 2.67 | 1.86E−04 | 1.64E−03 |
| Protein CYR61 | CCN1 | O00622 | 1.75 | 1.11 | 2.39 | 9.33E−08 | 1.83E−06 |
| Stanniocalcin-1 | STC1 | P52823 | 1.74 | 0.99 | 2.48 | 5.18E−06 | 6.80E−05 |
| Small ubiquitin-related modifier 2 | SUMO2 | P61956 | 1.73 | 1.16 | 2.3 | 2.53E−09 | 6.68E−08 |
| Complement C3b, inactivated | C3 | P01024 | 1.73 | 1.17 | 2.28 | 1.45E−09 | 4.10E−08 |
| HEPACAM family member 2: Isoform 1, | HEPACAM2 | A8MVW5 | 1.73 | 0.92 | 2.54 | 2.73E−05 | 3.09E−04 |
| Cytoplasmic domain | | | | | | | |
| Segment polarity protein dishevelled | DVL2 | O14641 | 1.72 | 0.72 | 2.72 | 7.62E−04 | 5.69E−03 |
| homolog DVL-2 | | | | | | | |
| Interleukin-18 | IL18 | Q14116 | 1.72 | 1.12 | 2.33 | 1.97E−08 | 4.36E−07 |
| Protein-tyrosine sulfotransferase 1 | TPST1 | O60507 | 1.72 | 0.74 | 2.69 | 5.41E−04 | 4.23E−03 |
| BMP-binding endothelial regulator protein | BMPER | Q8N8U9 | 1.71 | 0.84 | 2.59 | 1.26E−04 | 1.18E−03 |
| Hepatoma-derived growth factor | HDGF | P51858 | 1.71 | 0.98 | 2.44 | 4.89E−06 | 6.48E−05 |
| WNT1-inducible-signaling pathway protein 1 | CCN4 | O95388 | 1.71 | 0.83 | 2.6 | 1.55E−04 | 1.41E−03 |
| Semaphorin-6B | SEMA6B | Q9H3T3 | 1.71 | 0.77 | 2.64 | 3.50E−04 | 2.87E−03 |
| IGF-like family receptor 1 | IGFLR1 | Q9H665 | 1.71 | 1.17 | 2.24 | 4.15E−10 | 1.25E−08 |
| Acyl-CoA-binding protein | DBI | P07108 | 1.7 | 1.16 | 2.25 | 9.60E−10 | 2.79E−08 |
| Ephrin-B3: Extracellular domain | EFNB3 | Q15768 | 1.7 | 0.82 | 2.58 | 1.63E−04 | 1.45E−03 |
| Coagulation factor XIII B chain | F13B | P05160 | 1.7 | 0.58 | 2.83 | 2.94E−03 | 1.78E−02 |
| Glucokinase | GCK | P35557 | 1.69 | 0.47 | 2.92 | 6.78E−03 | 3.59E−02 |
| Tenascin | TNC | P24821 | 1.69 | 0.88 | 2.49 | 4.13E−05 | 4.38E−04 |
| Phospholipase B-like 1 | PLBD1 | Q6P4A8 | 1.69 | 1 | 2.38 | 1.71E−06 | 2.55E−05 |
| Histone H2A type 3 | H2AW | Q7L7L0 | 1.68 | 1.13 | 2.24 | 3.41E−09 | 8.66E−08 |
| DnaJ homolog subfamily B member 12 | DNAJB12 | Q9NXW2 | 1.68 | 0.98 | 2.39 | 3.01E−06 | 4.19E−05 |
| Complement component C9 | C9 | P02748 | 1.67 | 0.88 | 2.46 | 3.52E−05 | 3.85E−04 |
| N-acetyltransferase 14 | NAT14 | Q8WUY8 | 1.66 | 0.48 | 2.84 | 5.71E−03 | 3.13E−02 |
| Triggering receptor expressed on myeloid | TREM1 | Q9NP99 | 1.66 | 1.02 | 2.29 | 3.94E−07 | 6.73E−06 |
| cells 1 | | | | | | | |
| Structural maintenance of chromosomes | SMC3 | Q9UQE7 | 1.65 | 1.01 | 2.3 | 4.64E−07 | 7.78E−06 |
| protein 3 | | | | | | | |
| Transcription factor MafG | MAFG | O15525 | 1.65 | 0.87 | 2.43 | 3.65E−05 | 3.95E−04 |
| Collagen alpha-1(XV) chain | COL15A1 | P39059 | 1.65 | 0.65 | 2.65 | 1.22E−03 | 8.48E−03 |
| Enhancer of rudimentary homolog | ERH | P84090 | 1.64 | 1.06 | 2.22 | 2.71E−08 | 5.83E−07 |
| Growth/differentiation factor 9 | GDF9 | O60383 | 1.64 | 0.94 | 2.34 | 4.91E−06 | 6.49E−05 |
| Protocadherin-8 | PCDH8 | O95206 | 1.63 | 0.95 | 2.31 | 2.87E−06 | 4.01E−05 |
| Oxytocin-neurophysin 1 | OXT | P01178 | 1.63 | 1.2 | 2.07 | 2.78E−13 | 1.85E−11 |
| Growth hormone receptor | GHR | P10912 | 1.62 | 1.04 | 2.19 | 4.81E−08 | 9.98E−07 |
| Properdin | CFP | P27918 | 1.62 | 0.59 | 2.65 | 1.97E−03 | 1.28E−02 |
| Thrombospondin-2 | THBS2 | P35442 | 1.62 | 1.12 | 2.12 | 1.92E−10 | 6.29E−09 |
| Protein S100-A12 | S100A12 | P80511 | 1.62 | 0.99 | 2.24 | 3.95E−07 | 6.73E−06 |
| Endoplasmic reticulum lectin 1 | ERLEC1 | Q96DZ1 | 1.62 | 0.77 | 2.47 | 1.85E−04 | 1.63E−03 |
| C-X-C motif chemokine 13 | CXCL13 | O43927 | 1.61 | 0.96 | 2.26 | 1.21E−06 | 1.84E−05 |
| Heat shock factor protein 1 | HSF1 | Q00613 | 1.6 | 0.46 | 2.74 | 5.93E−03 | 3.23E−02 |
| C-C motif chemokine 14 | CCL14 | Q16627 | 1.6 | 1.15 | 2.06 | 4.44E−12 | 2.11E−10 |
| Galectin-4 | LGALS4 | P56470 | 1.6 | 0.73 | 2.48 | 3.22E−04 | 2.67E−03 |
| Cell growth regulator with EF hand domain | CGREF1 | Q99674 | 1.6 | 0.58 | 2.61 | 2.09E−03 | 1.34E−02 |
| protein 1 | | | | | | | |
| Programmed cell death protein 5 | PDCD5 | O14737 | 1.59 | 0.97 | 2.21 | 5.76E−07 | 9.41E−06 |
| C-X-C motif chemokine 14 | CXCL14 | O95715 | 1.59 | 0.65 | 2.53 | 9.31E−04 | 6.78E−03 |
| Zinc fingers and homeoboxes protein 3 | ZHX3 | Q9H412 | 1.58 | 0.49 | 2.68 | 4.49E−03 | 2.56E−02 |
| Transaldolase | TALDO1 | P37837 | 1.58 | 0.84 | 2.32 | 3.11E−05 | 3.48E−04 |
| 60S ribosomal protein L30 | RPL30 | P62888 | 1.58 | 1.07 | 2.09 | 1.25E−09 | 3.58E−08 |
| C-C motif chemokine 7 | CCL7 | P80098 | 1.58 | 0.67 | 2.49 | 6.52E−04 | 4.99E−03 |
| NTF2-related export protein 1 | NXT1 | Q9UKK6 | 1.58 | 0.86 | 2.3 | 1.83E−05 | 2.15E−04 |
| Cellular retinoic acid-binding protein 2 | CRABP2 | P29373 | 1.57 | 0.9 | 2.24 | 4.47E−06 | 5.99E−05 |
| Toll-like receptor 1: Extracellular domain | TLR1 | Q15399 | 1.57 | 0.62 | 2.52 | 1.18E−03 | 8.24E−03 |
| Angiopoietin-2 | ANGPT2 | O15123 | 1.57 | 0.99 | 2.15 | 1.00E−07 | 1.95E−06 |
| Receptor-type tyrosine-protein phosphatase U | PTPRU | Q92729 | 1.57 | 0.91 | 2.24 | 3.74E−06 | 5.11E−05 |
| Guanine nucleotide-binding protein G(I)/G(S)/ | GNGT2 | O14610 | 1.56 | 0.81 | 2.31 | 4.44E−05 | 4.68E−04 |
| G(O) subunit gamma-T2 | | | | | | | |
| Tudor-interacting repair regulator protein | NUDT16L1 | Q9BRJ7 | 1.56 | 1.02 | 2.11 | 2.13E−08 | 4.65E−07 |
| NGFI-A-binding protein 1 | NAB1 | Q13506 | 1.56 | 0.8 | 2.32 | 5.51E−05 | 5.65E−04 |
| Hemojuvelin | HJV | Q6ZVN8 | 1.56 | 0.38 | 2.74 | 9.70E−03 | 4.87E−02 |
| Carbohydrate sulfotransferase 12 | CHST12 | Q9NRB3 | 1.56 | 0.76 | 2.36 | 1.35E−04 | 1.25E−03 |
| Microfibrillar-associated protein 2 | MFAP2 | P55001 | 1.55 | 0.4 | 2.7 | 8.31E−03 | 4.28E−02 |
| Allograft inflammatory factor 1-like | AIF1L | Q9BQI0 | 1.55 | 0.51 | 2.6 | 3.58E−03 | 2.10E−02 |
| Myeloperoxidase | MPO | P05164 | 1.55 | 0.91 | 2.2 | 2.48E−06 | 3.53E−05 |
| Alpha-1-antichymotrypsin complex | SERPINA3 | P01011 | 1.55 | 0.81 | 2.29 | 4.14E−05 | 4.38E−04 |
| Annexin A1 | ANXA1 | P04083 | 1.55 | 0.78 | 2.31 | 7.14E−05 | 7.22E−04 |
| Heat shock 70 kDa protein 1A | HSPA1A | P0DMV8 | 1.53 | 0.72 | 2.35 | 2.35E−04 | 2.01E−03 |
| Retinoblastoma-like protein 2 | RBL2 | Q08999 | 1.53 | 0.92 | 2.14 | 9.72E−07 | 1.51E−05 |
| SLAM family member 6 | SLAMF6 | Q96DU3 | 1.53 | 0.74 | 2.33 | 1.62E−04 | 1.45E−03 |
| Apolipoprotein L1 | APOL1 | O14791 | 1.52 | 0.58 | 2.47 | 1.64E−03 | 1.08E−02 |
| C-C motif chemokine 8 | CCL8 | P80075 | 1.52 | 0.9 | 2.13 | 1.38E−06 | 2.07E−05 |
| RNA polymerase-associated protein RTF1 | RTF1 | Q92541 | 1.52 | 0.58 | 2.46 | 1.60E−03 | 1.06E−02 |
| homolog | | | | | | | |
| Spliceosome RNA helicase DDX39B | DDX39B | Q13838 | 1.52 | 0.8 | 2.25 | 3.83E−05 | 4.10E−04 |
| Dolichyl-diphosphooligosaccharide--protein | RPN1 | P04843 | 1.51 | 0.87 | 2.15 | 4.33E−06 | 5.84E−05 |
| glycosyltransferase subunit 1: Cytoplasmic: | | | | | | | |
| domain | | | | | | | |
| Atrial natriuretic factor | NPPA | P01160 | 1.51 | 0.43 | 2.58 | 6.04E−03 | 3.27E−02 |
| A disintegrin and metalloproteinase with | ADAMTS6 | Q9UKP5 | 1.51 | 0.62 | 2.4 | 8.74E−04 | 6.43E−03 |
| thrombospondin motifs 6 | | | | | | | |
| Fumarylacetoacetase | FAH | P16930 | 1.5 | 0.88 | 2.11 | 1.86E−06 | 2.75E−05 |
| Erythropoietin receptor | EPOR | P19235 | 1.5 | 0.7 | 2.3 | 2.37E−04 | 2.02E−03 |
| Protein kinase C gamma type | PRKCG | P05129 | 1.49 | 0.76 | 2.23 | 6.92E−05 | 7.05E−04 |
| Protein dpy-30 homolog | DPY30 | Q9C005 | 1.48 | 0.86 | 2.09 | 2.32E−06 | 3.34E−05 |
| Ephrin type-B receptor 2 | EPHB2 | P29323 | 1.48 | 0.54 | 2.43 | 2.00E−03 | 1.29E−02 |
| Olfactomedin-like protein 3 | OLFML3 | Q9NRN5 | 1.47 | 0.39 | 2.56 | 7.73E−03 | 4.01E−02 |
| Interleukin-21 receptor | IL21R | Q9HBE5 | 1.47 | 0.5 | 2.44 | 3.11E−03 | 1.87E−02 |
| Solute carrier family 35 member G2 | SLC35G2 | Q8TBE7 | 1.46 | 0.72 | 2.21 | 1.24E−04 | 1.16E−03 |
| Angiogenin | ANG | P03950 | 1.46 | 0.69 | 2.22 | 1.90E−04 | 1.67E−03 |
| Vitrin | VIT | Q6UXI7 | 1.46 | 0.69 | 2.24 | 2.21E−04 | 1.91E−03 |
| Signaling lymphocytic activation molecule | SLAMF1 | Q13291 | 1.46 | 0.75 | 2.17 | 5.13E−05 | 5.35E−04 |
| Syndecan-3 | SDC3 | O75056 | 1.45 | 0.75 | 2.16 | 5.71E−05 | 5.83E−04 |
| Protein Mdm4 | MDM4 | O15151 | 1.44 | 0.86 | 2.03 | 1.23E−06 | 1.85E−05 |
| Pappalysin-2 | PAPPA2 | Q9BXP8 | 1.44 | 0.42 | 2.46 | 5.70E−03 | 3.13E−02 |
| Parathyroid hormone | PTH | P01270 | 1.44 | 0.96 | 1.92 | 5.67E−09 | 1.41E−07 |
| Collagen alpha-1(XX) chain | COL20A1 | Q9P218 | 1.44 | 0.65 | 2.23 | 3.72E−04 | 3.02E−03 |
| Sushi, von Willebrand factor type A, EGF and | SVEP1 | Q4LDE5 | 1.43 | 0.69 | 2.17 | 1.58E−04 | 1.43E−03 |
| pentraxin domain-containing protein 1: Sushi | | | | | | | |
| 15-18 | | | | | | | |
| Thioredoxin domain-containing protein 5 | TXNDC5 | Q8NBS9 | 1.43 | 0.57 | 2.3 | 1.13E−03 | 7.92E−03 |
| Low affinity immunoglobulin epsilon Fc | FCER2 | P06734 | 1.43 | 0.88 | 1.97 | 2.91E−07 | 5.19E−06 |
| receptor | | | | | | | |
| Selenoprotein W | SELENOW | P63302 | 1.42 | 0.68 | 2.15 | 1.56E−04 | 1.42E−03 |
| 40S ribosomal protein S5 | RPS5 | P46782 | 1.42 | 0.6 | 2.24 | 7.02E−04 | 5.33E−03 |
| Rho guanine nucleotide exchange factor 10 | ARHGEF10 | O15013 | 1.42 | 0.39 | 2.45 | 6.73E−03 | 3.57E−02 |
| Macrophage scavenger receptor types I and | MSR1 | P21757 | 1.41 | 0.88 | 1.94 | 1.64E−07 | 3.04E−06 |
| II: Extracellular domain | | | | | | | |
| Glypican-4 | GPC4 | O75487 | 1.41 | 0.67 | 2.16 | 2.10E−04 | 1.83E−03 |
| C-C motif chemokine 25 | CCL25 | O15444 | 1.41 | 0.91 | 1.91 | 3.40E−08 | 7.21E−07 |
| C-X-C motif chemokine 10 | CXCL10 | P02778 | 1.41 | 0.91 | 1.92 | 4.50E−08 | 9.46E−07 |
| DOMON domain-containing protein FRRS1L | FRRS1L | Q9P0K9 | 1.4 | 0.86 | 1.94 | 3.59E−07 | 6.21E−06 |
| Hepatoma-derived growth factor | HDGF | P51858 | 1.4 | 0.69 | 2.12 | 1.19E−04 | 1.13E−03 |
| Complement factor D | CFD | P00746 | 1.39 | 0.37 | 2.42 | 7.71E−03 | 4.00E−02 |
| Bone morphogenetic protein 1 | BMP1 | P13497 | 1.39 | 0.6 | 2.18 | 5.47E−04 | 4.28E−03 |
| Fibronectin Fragment 3 | FN1 | P02751 | 1.39 | 0.68 | 2.11 | 1.43E−04 | 1.31E−03 |
| L-lactate dehydrogenase A chain | LDHA | P00338 | 1.38 | 0.68 | 2.07 | 1.04E−04 | 9.92E−04 |
| TATA-binding protein-associated factor 2N | TAF15 | Q92804 | 1.38 | 0.67 | 2.09 | 1.39E−04 | 1.28E−03 |
| Lysozyme C | LYZ | P61626 | 1.38 | 0.75 | 2.01 | 1.86E−05 | 2.17E−04 |
| GDNF family receptor alpha-like | GFRAL | Q6UXV0 | 1.38 | 0.66 | 2.1 | 1.72E−04 | 1.52E−03 |
| Axin-2 | AXIN2 | Q9Y2T1 | 1.38 | 0.94 | 1.83 | 9.13E−10 | 2.67E−08 |
| Netrin-4 | NTN4 | Q9HB63 | 1.37 | 0.49 | 2.25 | 2.23E−03 | 1.40E−02 |
| Ephrin type-A receptor 2 | EPHA2 | P29317 | 1.37 | 0.55 | 2.2 | 1.10E−03 | 7.76E−03 |
| 39S ribosomal protein L34, mitochondrial | MRPL34 | Q9BQ48 | 1.37 | 0.41 | 2.33 | 5.10E−03 | 2.85E−02 |
| Rho GTPase-activating protein 1: Cellular | ARHGAP1 | Q07960 | 1.37 | 0.75 | 1.98 | 1.48E−05 | 1.76E−04 |
| retinaldehyde-TRIO domain | | | | | | | |
| U6 snRNA phosphodiesterase | USB1 | Q9BQ65 | 1.36 | 0.41 | 2.31 | 4.83E−03 | 2.72E−02 |
| Progesterone receptor | PGR | P06401 | 1.36 | 0.67 | 2.04 | 9.89E−05 | 9.58E−04 |
| Holo-Transcobalamin-2 | TCN2 | P20062 | 1.36 | 0.55 | 2.16 | 9.30E−04 | 6.78E−03 |
| Procollagen-lysine, 2-oxoglutarate 5- | PLOD2 | O00469 | 1.36 | 0.75 | 1.98 | 1.27E−05 | 1.54E−04 |
| dioxygenase 2 | | | | | | | |
| UPF0577 protein KIAA1324: Cytoplasmic | ELAPOR1 | Q6UXG2 | 1.36 | 0.57 | 2.15 | 7.26E−04 | 5.46E−03 |
| domain | | | | | | | |
| Ena/VASP-like protein | EVL | Q9UI08 | 1.35 | 0.4 | 2.31 | 5.48E−03 | 3.02E−02 |
| Thioredoxin reductase 1, cytoplasmic | TXNRD1 | Q16881 | 1.35 | 0.82 | 1.88 | 5.50E−07 | 9.02E−06 |
| PC4 and SFRS1-interacting protein | PSIP1 | O75475 | 1.35 | 0.7 | 2.01 | 5.45E−05 | 5.62E−04 |
| B-cell differentiation antigen CD72 | CD72 | P21854 | 1.35 | 0.68 | 2.02 | 8.68E−05 | 8.53E−04 |
| Heat shock 70 kDa protein 1A | HSPA1A | P0DMV8 | 1.34 | 0.35 | 2.33 | 8.18E−03 | 4.21E−02 |
| Poly [ADP-ribose] polymerase 11 | PARP11 | Q9NR21 | 1.34 | 0.44 | 2.24 | 3.58E−03 | 2.10E−02 |
| Heat shock protein beta-6 | HSPB6 | O14558 | 1.34 | 0.69 | 1.99 | 5.43E−05 | 5.61E−04 |
| Liver-expressed antimicrobial peptide 2 | LEAP2 | Q969E1 | 1.34 | 0.83 | 1.84 | 2.10E−07 | 3.81E−06 |
| Heat shock 70 kDa protein 1A | HSPA1A | P0DMV8 | 1.33 | 0.55 | 2.11 | 7.95E−04 | 5.90E−03 |
| Annexin A2 | ANXA2 | P07355 | 1.33 | 0.48 | 2.18 | 2.08E−03 | 1.33E−02 |
| Serum amyloid A-2 protein | SAA2 | P0DJI9 | 1.33 | 0.97 | 1.69 | 7.00E−13 | 3.87E−11 |
| CMP-N-acetylneuraminate-beta-galactosamide- | ST3GAL2 | Q16842 | 1.33 | 0.75 | 1.9 | 5.89E−06 | 7.66E−05 |
| alpha-2,3-sialyltransferase 2 | | | | | | | |
| Heat shock 70 kDa protein 1A | HSPA1A | P0DMV8 | 1.33 | 0.67 | 2 | 8.65E−05 | 8.51E−04 |
| Heat shock 70 kDa protein 1A | HSPA1A | P0DMV8 | 1.32 | 0.62 | 2.02 | 2.16E−04 | 1.88E−03 |
| Sialoadhesin | SIGLEC1 | Q9BZZ2 | 1.32 | 0.79 | 1.85 | 1.18E−06 | 1.80E−05 |
| Erythropoietin | EPO | P01588 | 1.32 | 0.86 | 1.78 | 1.94E−08 | 4.30E−07 |
| Low-density lipoprotein receptor-related | LRP11 | Q86VZ4 | 1.32 | 0.42 | 2.22 | 4.05E−03 | 2.33E−02 |
| protein 11 | | | | | | | |
| Transmembrane emp24 domain-containing | TMED2 | Q15363 | 1.31 | 0.37 | 2.24 | 6.07E−03 | 3.28E−02 |
| protein 2 | | | | | | | |
| Rho GTPase-activating protein 1: Rho-GTPase | ARHGAP1 | Q07960 | 1.31 | 0.42 | 2.19 | 3.71E−03 | 2.17E−02 |
| activating protein domain | | | | | | | |
| Neutrophil elastase | ELANE | P08246 | 1.31 | 0.48 | 2.15 | 2.04E−03 | 1.31E−02 |
| Peptidyl-tRNA hydrolase ICT1, mitochondrial | MRPL58 | Q14197 | 1.31 | 0.47 | 2.15 | 2.20E−03 | 1.40E−02 |
| Transferrin receptor protein 1: Cytoplasmic | TFRC | P02786 | 1.31 | 0.82 | 1.79 | 1.26E−07 | 2.40E−06 |
| domain | | | | | | | |
| Voltage-gated potassium channel subunit beta-2 | KCNAB2 | Q13303 | 1.3 | 0.52 | 2.09 | 1.13E−03 | 7.95E−03 |
| Tissue-type plasminogen activator | PLAT | P00750 | 1.3 | 0.82 | 1.78 | 9.85E−08 | 1.92E−06 |
| Neuroblastoma suppressor of tumorigenicity 1 | NBL1 | P41271 | 1.3 | 0.56 | 2.04 | 6.27E−04 | 4.83E−03 |
| Lymphocyte antigen 6 complex locus protein | LY6G6C | O95867 | 1.3 | 0.71 | 1.89 | 1.77E−05 | 2.08E−04 |
| G6c | | | | | | | |
| Calcineurin subunit B type 1 | PPP3R1 | P63098 | 1.29 | 0.43 | 2.14 | 3.13E−03 | 1.88E−02 |
| Leukocyte cell-derived chemotaxin-2 | LECT2 | O14960 | 1.29 | 0.7 | 1.88 | 1.95E−05 | 2.26E−04 |
| Tripeptidyl-peptidase 1 | TPP1 | O14773 | 1.29 | 0.57 | 2.01 | 4.55E−04 | 3.61E−03 |
| Leukocyte-specific transcript 1 protein | LST1 | O00453 | 1.29 | 0.65 | 1.93 | 8.49E−05 | 8.39E−04 |
| Transmembrane protein 119 | TMEM119 | Q4V9L6 | 1.28 | 0.38 | 2.17 | 5.08E−03 | 2.84E−02 |
| Gastric inhibitory polypeptide | GIP | P09681 | 1.28 | 0.62 | 1.95 | 1.64E−04 | 1.46E−03 |
| Elafin | PI3 | P19957 | 1.28 | 0.77 | 1.79 | 8.47E−07 | 1.33E−05 |
| GSK3-beta interaction protein | GSKIP | Q9P0R6 | 1.27 | 0.66 | 1.89 | 5.38E−05 | 5.58E−04 |
| Serine/threonine-protein phosphatase 2A | PPP2R3A | Q06190 | 1.27 | 0.31 | 2.24 | 9.46E−03 | 4.78E−02 |
| regulatory subunit B″ subunit alpha | | | | | | | |
| Ubiquitin-conjugating enzyme E2 E1 | UBE2E1 | P51965 | 1.27 | 0.74 | 1.81 | 3.26E−06 | 4.50E−05 |
| Neuronal-specific septin-3 | SEPTIN3 | Q9UH03 | 1.27 | 0.48 | 2.05 | 1.58E−03 | 1.05E−02 |
| Heparanase | HPSE | Q9Y251 | 1.27 | 0.37 | 2.18 | 5.81E−03 | 3.17E−02 |
| Transmembrane emp24 domain-containing | TMED10 | P49755 | 1.26 | 0.53 | 1.99 | 6.89E−04 | 5.25E−03 |
| protein 10 | | | | | | | |
| Syntaxin-12 | STX12 | Q86Y82 | 1.25 | 0.68 | 1.83 | 1.92E−05 | 2.24E−04 |
| Fibronectin | FN1 | P02751 | 1.25 | 0.63 | 1.87 | 7.64E−05 | 7.66E−04 |
| Corneodesmosin | CDSN | Q15517 | 1.25 | 0.71 | 1.78 | 5.28E−06 | 6.92E−05 |
| Keratin, type I cytoskeletal 18 | KRT18 | P05783 | 1.24 | 0.51 | 1.97 | 8.84E−04 | 6.49E−03 |
| Guanylyl cyclase-activating protein 1 | GUCA1A | P43080 | 1.23 | 0.35 | 2.11 | 6.15E−03 | 3.32E−02 |
| Toll-like receptor 1: Cytoplasmic domain | TLR1 | Q15399 | 1.23 | 0.47 | 1.99 | 1.56E−03 | 1.03E−02 |
| Double-stranded RNA-binding protein Staufen | STAU1 | O95793 | 1.23 | 0.4 | 2.06 | 3.79E−03 | 2.21E−02 |
| homolog 1 | | | | | | | |
| NAD kinase | NADK | O95544 | 1.23 | 0.52 | 1.94 | 7.04E−04 | 5.34E−03 |
| Gamma-interferon-inducible protein 16: | IFI16 | Q16666 | 1.23 | 0.68 | 1.78 | 1.28E−05 | 1.55E−04 |
| Isoform 2, Hematopoietic expression, | | | | | | | |
| interferon-inducible nature, and nuclear | | | | | | | |
| localization 1 | | | | | | | |
| Stathmin | STMN1 | P16949 | 1.23 | 0.5 | 1.97 | 1.02E−03 | 7.31E−03 |
| Clathrin light chain A | CLTA | P09496 | 1.23 | 0.5 | 1.97 | 1.02E−03 | 7.31E−03 |
| Fibronectin Fragment 4 | FN1 | P02751 | 1.23 | 0.53 | 1.93 | 5.71E−04 | 4.44E−03 |
| Serine/threonine-protein kinase VRK1 | VRK1 | Q99986 | 1.22 | 0.3 | 2.15 | 9.60E−03 | 4.83E−02 |
| Galectin-3-binding protein | LGALS3BP | Q08380 | 1.22 | 0.75 | 1.68 | 3.03E−07 | 5.34E−06 |
| Ul small nuclear ribonucleoprotein A | SNRPA | P09012 | 1.21 | 0.56 | 1.87 | 2.91E−04 | 2.44E−03 |
| Interleukin-16 | IL16 | Q14005 | 1.21 | 0.38 | 2.03 | 4.21E−03 | 2.41E−02 |
| C-reactive protein | CRP | P02741 | 1.21 | 0.99 | 1.43 | 2.09E−26 | 1.04E−22 |
| C-C motif chemokine 19 | CCL19 | Q99731 | 1.21 | 0.7 | 1.73 | 4.40E−06 | 5.92E−05 |
| Spondin-2 | SPON2 | Q9BUD6 | 1.21 | 0.51 | 1.9 | 7.29E−04 | 5.48E−03 |
| Translocon-associated protein subunit alpha | SSR1 | P43307 | 1.21 | 0.51 | 1.92 | 7.10E−04 | 5.36E−03 |
| Thymocyte nuclear protein 1 | THYN1 | Q9P016 | 1.2 | 0.35 | 2.05 | 5.88E−03 | 3.20E−02 |
| Myc target protein 1 | MYCT1 | Q8N699 | 1.2 | 0.5 | 1.91 | 8.38E−04 | 6.20E−03 |
| Inhibin beta A chain | INHBA | P08476 | 1.2 | 0.59 | 1.81 | 1.03E−04 | 9.92E−04 |
| Histone H2B type 2-E | H2BC21 | Q16778 | 1.2 | 0.74 | 1.66 | 3.47E−07 | 6.04E−06 |
| Sialidase-1 | NEU1 | Q99519 | 1.2 | 0.58 | 1.81 | 1.45E−04 | 1.32E−03 |
| Activated RNA polymerase II transcriptional | SUB1 | P53999 | 1.2 | 0.52 | 1.88 | 5.17E−04 | 4.05E−03 |
| coactivator p15 | | | | | | | |
| Myosin light chain 6B | MYL6B | P14649 | 1.19 | 0.48 | 1.89 | 9.64E−04 | 6.99E−03 |
| Ubiquitin + 1, truncated mutation for UbB | RPS27A | P62979 | 1.19 | 0.53 | 1.84 | 3.94E−04 | 3.16E−03 |
| Cadherin-2 | CDH2 | P19022 | 1.19 | 0.54 | 1.84 | 3.65E−04 | 2.98E−03 |
| Interleukin-6 | IL6 | P05231 | 1.19 | 0.64 | 1.74 | 2.22E−05 | 2.54E−04 |
| DNA-(apurinic or apyrimidinic site) lyase | APEX1 | P27695 | 1.19 | 0.63 | 1.76 | 3.65E−05 | 3.95E−04 |
| Cathepsin Z | CTSZ | Q9UBR2 | 1.18 | 0.41 | 1.96 | 2.76E−03 | 1.68E−02 |
| CXADR-like membrane protein: Extracellular | CLMP | Q9H6B4 | 1.17 | 0.55 | 1.8 | 2.36E−04 | 2.02E−03 |
| domain | | | | | | | |
| DNA-directed RNA polymerase III subunit | POLR3F | Q9H1D9 | 1.17 | 0.34 | 1.99 | 5.75E−03 | 3.15E−02 |
| RPC6 | | | | | | | |
| Vascular endothelial growth factor D | VEGFD | O43915 | 1.17 | 0.69 | 1.64 | 1.74E−06 | 2.58E−05 |
| Uridine phosphorylase 1 | UPP1 | Q16831 | 1.17 | 0.4 | 1.93 | 2.69E−03 | 1.65E−02 |
| Proto-oncogene tyrosine-protein kinase | RET | P07949 | 1.17 | 0.67 | 1.67 | 4.31E−06 | 5.83E−05 |
| receptor Ret | | | | | | | |
| Carbonic anhydrase 3 | CA3 | P07451 | 1.17 | 0.73 | 1.6 | 1.86E−07 | 3.41E−06 |
| Matrix metalloproteinase-19 | MMP19 | Q99542 | 1.17 | 0.43 | 1.91 | 1.92E−03 | 1.25E−02 |
| Protein ripply1 | RIPPLY1 | Q0D2K3 | 1.17 | 0.28 | 2.06 | 9.81E−03 | 4.92E−02 |
| ATP synthase subunit f, mitochondrial | ATP5MF | P56134 | 1.16 | 0.45 | 1.87 | 1.39E−03 | 9.41E−03 |
| Troponin T, cardiac muscle | TNNT2 | P45379 | 1.16 | 0.65 | 1.66 | 8.37E−06 | 1.06E−04 |
| SLAM family member 7 | SLAMF7 | Q9NQ25 | 1.16 | 0.77 | 1.56 | 6.20E−09 | 1.52E−07 |
| Glutathione S-transferase A1 | GSTA1 | P08263 | 1.15 | 0.63 | 1.66 | 1.25E−05 | 1.53E−04 |
| Heterogeneous nuclear ribonucleoprotein D0 | HNRNPD | Q14103 | 1.15 | 0.47 | 1.84 | 9.65E−04 | 6.99E−03 |
| Guanylate-binding protein 2 | GBP2 | P32456 | 1.15 | 0.49 | 1.81 | 6.83E−04 | 5.21E−03 |
| 40S ribosomal protein S19 | RPS19 | P39019 | 1.15 | 0.44 | 1.86 | 1.48E−03 | 9.87E−03 |
| Glycerol-3-phosphate dehydrogenase | GPD1 | P21695 | 1.14 | 0.71 | 1.56 | 1.64E−07 | 3.04E−06 |
| [NAD(+)], cytoplasmic | | | | | | | |
| Cadherin-11: Extracellular domain | CDH11 | P55287 | 1.14 | 0.85 | 1.42 | 1.59E−14 | 1.36E−12 |
| Prostaglandin reductase 1 | PTGR1 | Q14914 | 1.14 | 0.75 | 1.53 | 1.04E−08 | 2.43E−07 |
| Transferrin receptor protein 1: Extracellular | TFRC | P02786 | 1.14 | 0.53 | 1.75 | 2.36E−04 | 2.02E−03 |
| domain | | | | | | | |
| Neuregulin-1, sensory and motor neuron- | NRG1 | Q02297 | 1.13 | 0.3 | 1.95 | 7.43E−03 | 3.87E−02 |
| derived factor isoform | | | | | | | |
| Alpha-(1,6)-fucosyltransferase | FUT8 | Q9BYC5 | 1.13 | 0.51 | 1.76 | 3.75E−04 | 3.04E−03 |
| Cadherin-related family member 1 | CDHR1 | Q96JP9 | 1.13 | 0.41 | 1.86 | 2.28E−03 | 1.43E−02 |
| Fas apoptotic inhibitory molecule 3 | FCMR | O60667 | 1.12 | 0.58 | 1.66 | 5.69E−05 | 5.82E−04 |
| CUGBP Elav-like family member 2 | CELF2 | O95319 | 1.12 | 0.4 | 1.85 | 2.33E−03 | 1.46E−02 |
| Platelet-derived growth factor D | PDGFD | Q9GZP0 | 1.12 | 0.42 | 1.83 | 1.78E−03 | 1.16E−02 |
| Anaphase-promoting complex subunit 7 | ANAPC7 | Q9UJX3 | 1.11 | 0.27 | 1.95 | 9.49E−03 | 4.79E−02 |
| Hexokinase-2 | HK2 | P52789 | 1.11 | 0.45 | 1.76 | 9.27E−04 | 6.78E−03 |
| Galactosylgalactosylxylosylprotein 3-beta- | B3GAT3 | O94766 | 1.11 | 0.34 | 1.89 | 4.99E−03 | 2.80E−02 |
| glucuronosyltransferase 3 | | | | | | | |
| Stathmin-3 | STMN3 | Q9NZ72 | 1.11 | 0.34 | 1.88 | 4.79E−03 | 2.70E−02 |
| Cystatin-8 | CST8 | O60676 | 1.1 | 0.49 | 1.71 | 3.91E−04 | 3.15E−03 |
| Histone H2B type 3-B | H2BU1 | Q8N257 | 1.1 | 0.7 | 1.5 | 9.15E−08 | 1.80E−06 |
| Microtubule-associated protein tau | MAPT | P10636 | 1.1 | 0.36 | 1.84 | 3.41E−03 | 2.02E−02 |
| Macrophage receptor MARCO | MARCO | Q9UEW3 | 1.1 | 0.34 | 1.85 | 4.40E−03 | 2.52E−02 |
| Pleckstrin homology domain-containing | PLEKHA7 | Q6IQ23 | 1.09 | 0.37 | 1.81 | 2.86E−03 | 1.74E−02 |
| family A member 7 | | | | | | | |
| Acylphosphatase-2 | ACYP2 | P14621 | 1.09 | 0.51 | 1.66 | 2.02E−04 | 1.77E−03 |
| Coagulation Factor VIII | F8 | P00451 | 1.09 | 0.43 | 1.75 | 1.24E−03 | 8.61E−03 |
| Macrophage colony-stimulating factor 1 | CSF1R | P07333 | 1.09 | 0.36 | 1.83 | 3.65E−03 | 2.14E−02 |
| receptor | | | | | | | |
| Induced myeloid leukemia cell differentiation | MCL1 | Q07820 | 1.08 | 0.52 | 1.65 | 1.58E−04 | 1.43E−03 |
| protein Mcl-1 | | | | | | | |
| Homeobox protein DLX-3 | DLX3 | O60479 | 1.08 | 0.47 | 1.68 | 4.71E−04 | 3.71E−03 |
| Ovarian cancer G-protein coupled receptor 1 | GPR68 | Q15743 | 1.08 | 0.33 | 1.83 | 4.73E−03 | 2.68E−02 |
| RNA-binding protein 3 | RBM3 | P98179 | 1.08 | 0.58 | 1.57 | 2.22E−05 | 2.54E−04 |
| SPARC-related modular calcium-binding | SMOC2 | Q9H3U7 | 1.08 | 0.33 | 1.83 | 4.89E−03 | 2.75E−02 |
| protein 2 | | | | | | | |
| C-C motif chemokine 15 | CCL15 | Q16663 | 1.08 | 0.52 | 1.64 | 1.59E−04 | 1.43E−03 |
| Cartilage intermediate layer protein 1 | CILP | O75339 | 1.08 | 0.53 | 1.62 | 1.06E−04 | 1.01E−03 |
| Polypeptide N-acetylgalactosaminyltransferase | GALNT2 | Q10471 | 1.08 | 0.45 | 1.72 | 8.51E−04 | 6.28E−03 |
| 2 | | | | | | | |
| NKG2-E type II integral membrane protein: | KLRC3 | Q07444 | 1.08 | 0.4 | 1.77 | 2.04E−03 | 1.31E−02 |
| Isoform H, Cytoplasmic domain | | | | | | | |
| Testin | TES | Q9UGI8 | 1.07 | 0.34 | 1.79 | 3.96E−03 | 2.28E−02 |
| Secretin | SCT | P09683 | 1.07 | 0.33 | 1.81 | 4.77E−03 | 2.70E−02 |
| Granulocyte colony-stimulating factor | CSF3 | P09919 | 1.07 | 0.63 | 1.52 | 2.48E−06 | 3.53E−05 |
| Fragile X mental retardation protein 1 | FMR1 | Q06787 | 1.07 | 0.38 | 1.75 | 2.44E−03 | 1.52E−02 |
| Ferritin, mitochondrial | FTMT | Q8N4E7 | 1.07 | 0.67 | 1.46 | 1.50E−07 | 2.83E−06 |
| Cyclin-H | CCNH | P51946 | 1.07 | 0.42 | 1.72 | 1.26E−03 | 8.69E−03 |
| Prokineticin-2 | PROK2 | Q9HC23 | 1.06 | 0.71 | 1.4 | 2.12E−09 | 5.73E−08 |
| Spermatogenesis-associated protein 20 | SPATA20 | Q8TB22 | 1.06 | 0.51 | 1.6 | 1.44E−04 | 1.32E−03 |
| Interferon-induced GTP-binding protein Mx1 | MX1 | P20591 | 1.06 | 0.59 | 1.54 | 1.31E−05 | 1.58E−04 |
| Macrophage metalloelastase | MMP12 | P39900 | 1.06 | 0.55 | 1.58 | 5.46E−05 | 5.62E−04 |
| Beta-defensin 135 | DEFB135 | Q30KP9 | 1.06 | 0.31 | 1.81 | 5.66E−03 | 3.11E−02 |
| Retinol-binding protein 5 | RBP5 | P82980 | 1.05 | 0.44 | 1.65 | 7.33E−04 | 5.49E−03 |
| 40S ribosomal protein SA | RPSA | P08865 | 1.05 | 0.41 | 1.69 | 1.29E−03 | 8.85E−03 |
| Tumor necrosis factor receptor superfamily | TNFRSF17 | Q02223 | 1.05 | 0.29 | 1.8 | 6.71E−03 | 3.56E−02 |
| member 17 | | | | | | | |
| 3-oxo-5-beta-steroid 4-dehydrogenase | AKR1D1 | P51857 | 1.04 | 0.56 | 1.51 | 2.06E−05 | 2.37E−04 |
| 40S ribosomal protein S3a | RPS3A | P61247 | 1.04 | 0.38 | 1.71 | 2.18E−03 | 1.39E−02 |
| Interleukin-31 receptor subunit alpha | IL31RA | Q8NI17 | 1.04 | 0.27 | 1.81 | 7.85E−03 | 4.06E−02 |
| [Pyruvate dehydrogenase (acetyl- | PDK2 | Q15119 | 1.03 | 0.34 | 1.71 | 3.27E−03 | 1.95E−02 |
| transferring)] kinase isozyme 2, mitochondrial | | | | | | | |
| Sperm surface protein Sp17 | SPA17 | Q15506 | 1.02 | 0.24 | 1.8 | 1.00E−02 | 4.99E−02 |
| Protein-tyrosine kinase 6 | PTK6 | Q13882 | 1.02 | 0.4 | 1.64 | 1.24E−03 | 8.61E−03 |
| Complement C3b | C3 | P01024 | 1.02 | 0.74 | 1.29 | 6.83E−13 | 3.84E−11 |
| Fructose-2,6-bisphosphatase TIGAR | TIGAR | Q9NQ88 | 1.01 | 0.39 | 1.62 | 1.30E−03 | 8.92E−03 |
| Inhibin beta B chain | INHBB | P09529 | 1.01 | 0.43 | 1.59 | 6.45E−04 | 4.95E−03 |
| Methyl-CpG-binding domain protein 1 | MBD1 | Q9UIS9 | 1.01 | 0.38 | 1.64 | 1.71E−03 | 1.13E−02 |
| Serum amyloid A-1 protein | SAA1 | PODJI8 | 1.01 | 0.77 | 1.25 | 2.48E−16 | 3.17E−14 |
| Beta-klotho | KLB | Q86Z14 | 1.01 | 0.3 | 1.72 | 5.52E−03 | 3.04E−02 |
| Thiosulfate sulfurtransferase | TST | Q16762 | 1 | 0.56 | 1.44 | 8.67E−06 | 1.09E−04 |
| Ubiquitin-conjugating enzyme E2 S | UBE2S | Q16763 | 1 | 0.42 | 1.59 | 7.38E−04 | 5.52E−03 |
| Protocadherin gamma-C3 | PCDHGC3 | Q9UN70 | 1 | 0.5 | 1.51 | 1.03E−04 | 9.89E−04 |
| Ribosome-binding protein 1 | RRBP1 | Q9P2E9 | 1 | 0.26 | 1.75 | 8.39E−03 | 4.31E−02 |
| Amyloid-like protein 2 | APLP2 | Q06481 | 0.99 | 0.24 | 1.75 | 9.83E−03 | 4.92E−02 |
| Protein jagged-2 | JAG2 | Q9Y219 | 0.99 | 0.34 | 1.65 | 2.87E−03 | 1.74E−02 |
| StAR-related lipid transfer protein 5 | STARD5 | Q9NSY2 | 0.98 | 0.31 | 1.65 | 4.11E−03 | 2.36E−02 |
| Granzyme B | GZMB | P10144 | 0.98 | 0.28 | 1.69 | 6.01E−03 | 3.26E−02 |
| Scavenger receptor cysteine-rich type 1 | CD163 | Q86VB7 | 0.98 | 0.38 | 1.59 | 1.46E−03 | 9.76E−03 |
| protein M130 | | | | | | | |
| GDP-fucose protein O-fucosyltransferase 1 | POFUT1 | Q9H488 | 0.98 | 0.28 | 1.67 | 5.96E−03 | 3.24E−02 |
| Torsin-1 A-interacting protein 1: Perinuclear | TOR1AIP1 | Q5JTV8 | 0.97 | 0.29 | 1.66 | 5.49E−03 | 3.02E−02 |
| domain | | | | | | | |
| UDP-glucose 4-epimerase | GALE | Q14376 | 0.97 | 0.27 | 1.67 | 6.63E−03 | 3.53E−02 |
| SH3 and multiple ankyrin repeat domains | SHANK3 | Q9BYB0 | 0.97 | 0.4 | 1.55 | 9.43E−04 | 6.85E−03 |
| protein 3 | | | | | | | |
| Selenide, water dikinase 1 | SEPHS1 | P49903 | 0.97 | 0.41 | 1.52 | 7.13E−04 | 5.37E−03 |
| Interleukin-8 | CXCL8 | P10145 | 0.97 | 0.37 | 1.56 | 1.42E−03 | 9.54E−03 |
| ADM2 | ADM2 | Q7Z4H4 | 0.97 | 0.53 | 1.41 | 1.61E−05 | 1.90E−04 |
| Titin | TTN | Q8WZ42 | 0.96 | 0.35 | 1.58 | 2.12E−03 | 1.35E−02 |
| Syntaxin-3 | STX3 | Q13277 | 0.96 | 0.31 | 1.61 | 3.88E−03 | 2.25E−02 |
| Activin A | INHBA | P08476 | 0.95 | 0.37 | 1.53 | 1.37E−03 | 9.27E−03 |
| C-C motif chemokine 16 | CCL16 | O15467 | 0.95 | 0.55 | 1.35 | 2.87E−06 | 4.01E−05 |
| High affinity cAMP-specific 3′,5′-cyclic | PDE7A | Q13946 | 0.95 | 0.24 | 1.67 | 9.14E−03 | 4.65E−02 |
| phosphodiesterase 7A | | | | | | | |
| Calreticulin | CALR | P27797 | 0.95 | 0.34 | 1.56 | 2.38E−03 | 1.48E−02 |
| Leptin | LEP | P41159 | 0.95 | 0.74 | 1.16 | 2.08E−18 | 4.71E−16 |
| Heterogeneous nuclear ribonucleoprotein Al | HNRNPA1 | P09651 | 0.94 | 0.49 | 1.38 | 3.54E−05 | 3.86E−04 |
| Ubiquitin-like protein ISG15 | ISG15 | P05161 | 0.94 | 0.53 | 1.35 | 6.11E−06 | 7.91E−05 |
| von Willebrand factor | VWF | P04275 | 0.94 | 0.47 | 1.41 | 8.34E−05 | 8.27E−04 |
| Integrin alpha-I: beta-1 complex | ITGA1\|ITGB1 | P56199\|P05556 | 0.94 | 0.47 | 1.42 | 1.04E−04 | 9.95E−04 |
| Fibroblast growth factor 23 | FGF23 | Q9GZV9 | 0.94 | 0.48 | 1.4 | 5.51E−05 | 5.65E−04 |
| Immunoglobulin A | IGHA1\|IGHA2 | P01876\|P01877 | 0.93 | 0.46 | 1.41 | 1.27E−04 | 1.19E−03 |
| Malate dehydrogenase, cytoplasmic | MDH1 | P40925 | 0.93 | 0.36 | 1.5 | 1.32E−03 | 9.05E−03 |
| Lactoperoxidase | LPO | P22079 | 0.93 | 0.49 | 1.36 | 3.24E−05 | 3.61E−04 |
| Thrombospondin type-1 domain-containing | THSD7A | Q9UPZ6 | 0.92 | 0.46 | 1.38 | 9.43E−05 | 9.21E−04 |
| protein 7A: Thrombospondin type-1 domain 6 | | | | | | | |
| Alpha-internexin | INA | Q16352 | 0.92 | 0.28 | 1.57 | 5.15E−03 | 2.87E−02 |
| Somatoliberin | GHRH | P01286 | 0.92 | 0.31 | 1.53 | 3.22E−03 | 1.93E−02 |
| Neutrophil cytosol factor 4 | NCF4 | Q15080 | 0.91 | 0.39 | 1.43 | 5.88E−04 | 4.56E−03 |
| ALK and LTK ligand 2 | ALKAL2 | Q6UX46 | 0.91 | 0.35 | 1.47 | 1.36E−03 | 9.24E−03 |
| Bromodomain-containing protein 2 | BRD2 | P25440 | 0.9 | 0.24 | 1.56 | 7.61E−03 | 3.96E−02 |
| Interleukin-12 subunit beta | IL12B | P29460 | 0.9 | 0.35 | 1.45 | 1.38E−03 | 9.32E−03 |
| Interferon alpha-16 | IFNA16 | P05015 | 0.9 | 0.35 | 1.44 | 1.32E−03 | 8.99E−03 |
| Forkhead box protein O3 | FOXO3 | O43524 | 0.89 | 0.28 | 1.51 | 4.65E−03 | 2.64E−02 |
| Growth/differentiation factor 15 | GDF15 | Q99988 | 0.89 | 0.37 | 1.4 | 8.37E−04 | 6.20E−03 |
| 4F2 cell-surface antigen heavy chain: | SLC3A2 | P08195 | 0.88 | 0.3 | 1.45 | 3.01E−03 | 1.81E−02 |
| Cytoplasmic domain | | | | | | | |
| ATP-dependent DNA helicase Q1 | RECQL | P46063 | 0.88 | 0.35 | 1.41 | 1.11E−03 | 7.82E−03 |
| N-acetyllactosaminide beta-1,3-N- | B3GNT4 | Q9C0J1 | 0.87 | 0.22 | 1.52 | 8.45E−03 | 4.34E−02 |
| acetylglucosaminyltransferase 4 | | | | | | | |
| Calretinin | CALB2 | P22676 | 0.87 | 0.51 | 1.23 | 2.11E−06 | 3.05E−05 |
| Syntaxin-10 | STX10 | O60499 | 0.86 | 0.33 | 1.39 | 1.53E−03 | 1.02E−02 |
| Hepatocyte growth factor | HGF | P14210 | 0.86 | 0.32 | 1.4 | 1.76E−03 | 1.15E−02 |
| Poly [ADP-ribose] polymerase 1: region 1 | PARP1 | P09874 | 0.86 | 0.32 | 1.41 | 1.97E−03 | 1.28E−02 |
| Solute carrier organic anion transporter family | SLCO5A1 | Q9H2Y9 | 0.85 | 0.34 | 1.35 | 1.09E−03 | 7.74E−03 |
| member 5A1 | | | | | | | |
| Fc receptor-like protein 1 | FCRL1 | Q96LA6 | 0.85 | 0.3 | 1.41 | 2.66E−03 | 1.63E−02 |
| 40S ribosomal protein S4, X isoform | RPS4X | P62701 | 0.85 | 0.35 | 1.35 | 8.74E−04 | 6.43E−03 |
| E3 ubiquitin-protein ligase ZNRF3 | ZNRF3 | Q9ULT6 | 0.84 | 0.22 | 1.46 | 7.60E−03 | 3.95E−02 |
| Ribonuclease UK114 | RIDA | P52758 | 0.84 | 0.36 | 1.33 | 6.51E−04 | 4.99E−03 |
| C-C motif chemokine 17 | CCL17 | Q92583 | 0.84 | 0.23 | 1.45 | 7.07E−03 | 3.72E−02 |
| Plasminogen activator inhibitor 1 | SERPINE1 | P05121 | 0.83 | 0.53 | 1.13 | 5.47E−08 | 1.13E−06 |
| Fibroblast growth factor 6 | FGF6 | P10767 | 0.83 | 0.24 | 1.43 | 6.28E−03 | 3.37E−02 |
| High affinity cGMP-specific 3′,5′-cyclic | PDE9A | O76083 | 0.83 | 0.32 | 1.35 | 1.54E−03 | 1.02E−02 |
| phosphodiesterase 9A | | | | | | | |
| Transmembrane protein C1orf162 | C1orf162 | Q8NEQ5 | 0.83 | 0.5 | 1.16 | 7.73E−07 | 1.23E−05 |
| Golgi membrane protein 1 | GOLM1 | Q8NBJ4 | 0.83 | 0.41 | 1.25 | 1.27E−04 | 1.19E−03 |
| Tapasin | TAPBP | O15533 | 0.82 | 0.46 | 1.18 | 6.33E−06 | 8.17E−05 |
| Alcohol dehydrogenase 1A | ADH1A | P07327 | 0.82 | 0.43 | 1.21 | 3.25E−05 | 3.61E−04 |
| Succinate-semialdehyde dehydrogenase, | ALDH5A1 | P51649 | 0.82 | 0.37 | 1.27 | 3.93E−04 | 3.16E−03 |
| mitochondrial | | | | | | | |
| Transcobalamin-1 | TCN1 | P20061 | 0.82 | 0.38 | 1.26 | 2.53E−04 | 2.14E−03 |
| Stathmin-4 | STMN4 | Q9H169 | 0.81 | 0.26 | 1.36 | 3.92E−03 | 2.26E−02 |
| Protein FAM209B | FAM209B | Q5JX69 | 0.81 | 0.27 | 1.36 | 3.40E−03 | 2.02E−02 |
| ERO1-like protein beta | ERO1B | Q86YB8 | 0.81 | 0.29 | 1.33 | 2.20E−03 | 1.40E−02 |
| Syntaxin-8 | STX8 | Q9UNK0 | 0.78 | 0.37 | 1.2 | 2.16E−04 | 1.88E−03 |
| Coiled-coil domain-containing protein 50 | CCDC50 | Q8IVM0 | 0.78 | 0.21 | 1.35 | 7.09E−03 | 3.73E−02 |
| Adapter molecule crk | CRK | P46108 | 0.77 | 0.27 | 1.27 | 2.40E−03 | 1.49E−02 |
| Proenkephalin-A | PENK | P01210 | 0.77 | 0.25 | 1.29 | 3.82E−03 | 2.22E−02 |
| SH3 domain-binding glutamic acid-rich-like | SH3BGRL | O75368 | 0.76 | 0.24 | 1.27 | 3.80E−03 | 2.21E−02 |
| protein | | | | | | | |
| Serine protease inhibitor Kazal-type 2 | SPINK2 | P20155 | 0.76 | 0.33 | 1.19 | 4.90E−04 | 3.85E−03 |
| Growth-regulated alpha protein | CXCL1 | P09341 | 0.75 | 0.38 | 1.13 | 8.63E−05 | 8.50E−04 |
| Histone H1.2 | H1-2 | P16403 | 0.75 | 0.27 | 1.23 | 2.33E−03 | 1.46E−02 |
| Phosphatidylethanolamine-binding protein 1 | PEBP1 | P30086 | 0.75 | 0.23 | 1.27 | 4.53E−03 | 2.57E−02 |
| Fructose-1,6-bisphosphatase 1 | FBP1 | P09467 | 0.75 | 0.37 | 1.12 | 8.25E−05 | 8.20E−04 |
| Glutamate receptor 4 | GRIA4 | P48058 | 0.74 | 0.23 | 1.25 | 4.51E−03 | 2.57E−02 |
| DnaJ homolog subfamily B member 4 | DNAJB4 | Q9UDY4 | 0.74 | 0.22 | 1.26 | 5.16E−03 | 2.87E−02 |
| Inhibin beta C chain | INHBC | P55103 | 0.73 | 0.34 | 1.13 | 2.48E−04 | 2.10E−03 |
| Beta-defensin 4A | DEFB4A | O15263 | 0.72 | 0.5 | 0.94 | 1.72E−10 | 5.78E−09 |
| Complement factor H-related protein 5 | CFHR5 | Q9BXR6 | 0.72 | 0.19 | 1.25 | 7.67E−03 | 3.98E−02 |
| Isopentenyl-diphosphate Delta-isomerase 1 | IDI1 | Q13907 | 0.72 | 0.23 | 1.21 | 3.85E−03 | 2.24E−02 |
| Alcohol dehydrogenase [NADP(+)] | AKR1A1 | P14550 | 0.72 | 0.21 | 1.24 | 5.85E−03 | 3.19E−02 |
| Pituitary adenylate cyclase-activating | ADCYAP1 | P18509 | 0.72 | 0.19 | 1.25 | 7.94E−03 | 4.10E−02 |
| polypeptide 27 | | | | | | | |
| Leukocyte immunoglobulin-like receptor | LILRB5 | O75023 | 0.7 | 0.21 | 1.2 | 5.23E−03 | 2.90E−02 |
| subfamily B member 5 | | | | | | | |
| Quinone oxidoreductase | CRYZ | Q08257 | 0.69 | 0.33 | 1.05 | 1.57E−04 | 1.43E−03 |
| U2 small nuclear ribonucleoprotein B″ | SNRPB2 | P08579 | 0.68 | 0.17 | 1.18 | 8.66E−03 | 4.43E−02 |
| Glutathione S-transferase Mu 1 | GSTM1 | P09488 | 0.67 | 0.25 | 1.09 | 1.97E−03 | 1.28E−02 |
| Beta-enolase | ENO3 | P13929 | 0.67 | 0.35 | 1 | 5.40E−05 | 5.59E−04 |
| Anterior gradient protein 3 | AGR3 | Q8TD06 | 0.67 | 0.38 | 0.95 | 4.07E−06 | 5.53E−05 |
| Lysosomal protective protein | CTSA | P10619 | 0.67 | 0.2 | 1.14 | 5.18E−03 | 2.88E−02 |
| Beta-glucuronidase | GUSB | P08236 | 0.66 | 0.3 | 1.03 | 3.80E−04 | 3.07E−03 |
| Thymidine phosphorylase | TYMP | P19971 | 0.65 | 0.22 | 1.08 | 2.83E−03 | 1.72E−02 |
| UBX domain-containing protein 2B | UBXN2B | Q14CS0 | 0.65 | 0.18 | 1.11 | 6.56E−03 | 3.50E−02 |
| Interferon regulatory factor 6 | IRF6 | O14896 | 0.65 | 0.24 | 1.06 | 2.04E−03 | 1.31E−02 |
| Nucleolysin TIAR | TIAL1 | Q01085 | 0.64 | 0.17 | 1.1 | 7.00E−03 | 3.69E−02 |
| Receptor-transporting protein 4 | RTP4 | Q96DX8 | 0.63 | 0.18 | 1.08 | 6.21E−03 | 3.34E−02 |
| Low affinity immunoglobulin gamma Fc region | FCGR2B | P31994 | 0.63 | 0.29 | 0.98 | 3.70E−04 | 3.01E−03 |
| receptor II-b | | | | | | | |
| Endoplasmic reticulum aminopeptidase 1 | ERAP1 | Q9NZ08 | 0.63 | 0.28 | 0.99 | 4.35E−04 | 3.48E−03 |
| Uncharacterized protein C1orf226 | C1orf226 | A1L170 | 0.63 | 0.21 | 1.05 | 3.25E−03 | 1.94E−02 |
| Collagen type IV alpha-3-binding protein: | CERT1 | Q9Y5P4 | 0.6 | 0.22 | 0.99 | 2.19E−03 | 1.39E−02 |
| StAR-related lipid-transfer domain, isoform 2 | | | | | | | |
| Chitinase-3-like protein 1 | CHI3L1 | P36222 | 0.58 | 0.35 | 0.8 | 5.92E−07 | 9.60E−06 |
| Probable RNA-binding protein 19 | RBM19 | Q9Y4C8 | 0.57 | 0.16 | 0.97 | 6.26E−03 | 3.37E−02 |
| Chitotriosidase-1 | CHIT1 | Q13231 | 0.56 | 0.19 | 0.93 | 2.88E−03 | 1.74E−02 |
| Troponin I, fast skeletal muscle | TNNI2 | P48788 | 0.56 | 0.23 | 0.89 | 8.52E−04 | 6.28E−03 |
| Hsp90alpha | HSP90AA1 | P07900 | 0.55 | 0.19 | 0.9 | 2.57E−03 | 1.59E−02 |
| Alkaline phosphatase, placental type | ALPP | P05187 | 0.55 | 0.23 | 0.86 | 6.93E−04 | 5.27E−03 |
| Signal-regulatory protein beta-1 | SIRPB1 | O00241 | 0.54 | 0.14 | 0.93 | 7.80E−03 | 4.03E−02 |
| Transmembrane protease serine 6 | TMPRSS6 | Q8IU80 | 0.53 | 0.13 | 0.92 | 9.27E−03 | 4.71E−02 |
| CD177 antigen | CD177 | Q8N6Q3 | 0.51 | 0.2 | 0.81 | 1.06E−03 | 7.53E−03 |
| Thyroglobulin | TG | P01266 | 0.5 | 0.23 | 0.76 | 2.64E−04 | 2.23E−03 |
| Interleukin-36 alpha | IL36A | Q9UHA7 | 0.49 | 0.15 | 0.83 | 5.12E−03 | 2.85E−02 |
| Leukocyte immunoglobulin-like receptor | LILRA3 | Q8N6C8 | 0.48 | 0.22 | 0.74 | 3.00E−04 | 2.50E−03 |
| subfamily A member 3 | | | | | | | |
| Myosin-binding protein C, slow-type | MYBPC1 | Q00872 | 0.48 | 0.17 | 0.8 | 2.58E−03 | 1.59E−02 |
| Cold-inducible RNA-binding protein | CIRBP | Q14011 | 0.46 | 0.14 | 0.78 | 5.22E−03 | 2.89E−02 |
| Myosin light chain 3 | MYL3 | P08590 | 0.46 | 0.14 | 0.78 | 5.10E−03 | 2.85E−02 |
| C-X-C motif chemokine 11 | CXCL11 | O14625 | 0.46 | 0.16 | 0.77 | 2.59E−03 | 1.59E−02 |
| Beta-2-microglobulin | B2M | P61769 | 0.44 | 0.15 | 0.73 | 3.00E−03 | 1.81E−02 |
| Alcohol dehydrogenase 4 | ADH4 | P08319 | 0.43 | 0.11 | 0.75 | 8.73E−03 | 4.46E−02 |
| Pre-mRNA-splicing factor RBM22 | RBM22 | Q9NW64 | 0.42 | 0.1 | 0.73 | 1.01E−02 | 5.00E−02 |
| Nucleoside diphosphate kinase, mitochondrial | NME4 | O00746 | 0.41 | 0.1 | 0.73 | 9.91E−03 | 4.96E−02 |
| Creatine kinase M-type: Creatine kinase B-type | CKB\|CKM | P12277\|P06732 | 0.4 | 0.15 | 0.65 | 1.93E−03 | 1.26E−02 |
| heterodimer | | | | | | | |
| Myomesin-2 | MYOM2 | P54296 | 0.38 | 0.11 | 0.66 | 5.75E−03 | 3.15E−02 |
| Zymogen granule membrane protein 16 | ZG16 | O60844 | 0.31 | 0.24 | 0.39 | 7.29E−16 | 8.25E−14 |
| Formimidoyltransferase-cyclodeaminase | FTCD | O95954 | 0.28 | 0.07 | 0.49 | 9.98E−03 | 4.98E−02 |
| Pregnancy zone protein | PZP | P20742 | 0.23 | 0.12 | 0.34 | 3.38E−05 | 3.72E−04 |
| Low affinity immunoglobulin gamma Fc region | FCGR2A | P12318 | 0.22 | 0.07 | 0.36 | 3.22E−03 | 1.93E−02 |
| receptor II-a | | | | | | | |
| GMP reductase 1 | GMPR | P36959 | 0.21 | 0.06 | 0.36 | 5.78E−03 | 3.16E−02 |
| Sialic acid-binding Ig-like lectin 9 | SIGLEC9 | Q9Y336 | 0.19 | 0.05 | 0.32 | 6.03E−03 | 3.27E−02 |
| Transcobalamin-2 | TCN2 | P20062 | −0.15 | −0.22 | −0.07 | 7.84E−05 | 7.86E−04 |
| Tapasin-related protein | TAPBPL | Q9BX59 | −0.29 | −0.49 | −0.1 | 3.75E−03 | 2.19E−02 |
| Low molecular weight phosphotyrosine protein | ACP1 | P24666 | −0.32 | −0.55 | −0.08 | 9.46E−03 | 4.78E−02 |
| phosphatase | | | | | | | |
| Leukocyte immunoglobulin-like receptor | LILRB1 | Q8NHL6 | −0.36 | −0.58 | −0.14 | 1.44E−03 | 9.70E−03 |
| subfamily B member 1 | | | | | | | |
| T-cell surface antigen CD2 | CD2 | P06729 | −0.48 | −0.66 | −0.3 | 1.62E−07 | 3.01E−06 |
| Mannose-binding protein C | MBL2 | P11226 | −0.51 | −0.85 | −0.17 | 3.43E−03 | 2.03E−02 |
| Apolipoprotein C-III | APOC3 | P02656 | −0.53 | −0.85 | −0.22 | 9.35E−04 | 6.81E−03 |
| Ribonucleoside-diphosphate reductase large | RRM1 | P23921 | −0.56 | −0.88 | −0.24 | 6.92E−04 | 5.27E−03 |
| subunit | | | | | | | |
| Protocadherin gamma-A1 | PCDHGA1 | Q9Y5H4 | −0.57 | −0.79 | −0.34 | 5.82E−07 | 9.47E−06 |
| Hepatocyte growth factor activator | HGFAC | Q04756 | −0.58 | −0.96 | −0.19 | 3.25E−03 | 1.94E−02 |
| Leucine-rich repeat-containing G-protein | LGR4 | Q9BXB1 | −0.59 | −1.01 | −0.17 | 6.17E−03 | 3.33E−02 |
| coupled receptor 4 | | | | | | | |
| Teratocarcinoma-derived growth factor 1 | TDGF1 | P13385 | −0.61 | −0.84 | −0.37 | 7.20E−07 | 1.15E−05 |
| Pancreatic triacylglycerol lipase | PNLIP | P16233 | −0.65 | −1.05 | −0.25 | 1.37E−03 | 9.31E−03 |
| Signal peptide, CUB and EGF-like domain- | SCUBE1 | Q8IWY4 | −0.65 | −0.98 | −0.32 | 1.27E−04 | 1.19E−03 |
| containing protein 1 | | | | | | | |
| Adhesion G protein-coupled receptor F5 | ADGRF5 | Q8IZF2 | −0.67 | −1.09 | −0.24 | 2.15E−03 | 1.37E−02 |
| Protein S100-A7 | S100A7 | P31151 | −0.67 | −1.03 | −0.31 | 2.96E−04 | 2.48E−03 |
| Lactase-phlorizin hydrolase | LCT | P09848 | −0.67 | −0.94 | −0.4 | 1.06E−06 | 1.63E−05 |
| Immunoglobulin M | IGHM | P01871 | −0.68 | −1.09 | −0.27 | 1.05E−03 | 7.44E−03 |
| Cytokine-like protein 1 | CYTL1 | Q9NRR1 | −0.69 | −1.13 | −0.25 | 2.23E−03 | 1.40E−02 |
| Leptin receptor, soluble | LEPR | P48357 | −0.7 | −1.08 | −0.33 | 2.20E−04 | 1.90E−03 |
| Low-density lipoprotein receptor-related | LRP1 | Q07954 | −0.7 | −1.22 | −0.17 | 9.40E−03 | 4.76E−02 |
| protein 1, soluble: Extracellular domain, | | | | | | | |
| LDL receptor class A domains 11-20 | | | | | | | |
| SUN domain-containing protein 5 | SUN5 | Q8TC36 | −0.72 | −1.25 | −0.19 | 7.34E−03 | 3.84E−02 |
| Pappalysin-1 | PAPPA | Q13219 | −0.72 | −1.16 | −0.29 | 1.06E−03 | 7.50E−03 |
| V-set and immunoglobulin domain-containing | VSIG10 | Q8N0Z9 | −0.74 | −1.29 | −0.19 | 8.76E−03 | 4.47E−02 |
| protein 10 | | | | | | | |
| Cornulin | CRNN | Q9UBG3 | −0.74 | −1.25 | −0.23 | 4.44E−03 | 2.54E−02 |
| Creatine kinase U-type, mitochondrial | CKMT1A | P12532 | −0.75 | −1.26 | −0.24 | 3.76E−03 | 2.19E−02 |
| Catalase | CAT | P04040 | −0.75 | −1.21 | −0.29 | 1.41E−03 | 9.49E−03 |
| Signal peptide, CUB and EGF-like domain- | SCUBE3 | Q8IX30 | −0.76 | −1.31 | −0.2 | 7.45E−03 | 3.88E−02 |
| containing protein 3 | | | | | | | |
| Protein O-linked-mannose beta-1,4-N- | POMGNT2 | Q8NAT1 | −0.76 | −1.29 | −0.22 | 5.34E−03 | 2.95E−02 |
| acetylglucosaminyltransferase 2 | | | | | | | |
| Interleukin-2 receptor subunit beta | IL2RB | P14784 | −0.76 | −1.31 | −0.2 | 7.40E−03 | 3.86E−02 |
| Secretoglobin family 3A member 1 | SCGB3A1 | Q96QR1 | −0.78 | −1.14 | −0.42 | 2.01E−05 | 2.32E−04 |
| Chondroadherin | CHAD | O15335 | −0.79 | −1.36 | −0.22 | 6.85E−03 | 3.62E−02 |
| Protein FAM234B: C-term | FAM234B | A2RU67 | −0.79 | −1.36 | −0.21 | 7.13E−03 | 3.75E−02 |
| Growth hormone variant | GH2 | P01242 | −0.8 | −1.08 | −0.52 | 3.01E−08 | 6.44E−07 |
| 39S ribosomal protein L33, mitochondrial | MRPL33 | O75394 | −0.8 | −1.38 | −0.22 | 6.61E−03 | 3.52E−02 |
| R-spondin-1 | RSPO1 | Q2MKA7 | −0.8 | −1.4 | −0.19 | 9.57E−03 | 4.82E−02 |
| Antibacterial protein LL-37 | CAMP | P49913 | −0.82 | −1.42 | −0.23 | 6.77E−03 | 3.59E−02 |
| Mucin-16 | MUC16 | Q8WXI7 | −0.84 | −1.42 | −0.26 | 4.77E−03 | 2.70E−02 |
| Platelet-derived growth factor receptor beta | PDGFRB | P09619 | −0.85 | −1.3 | −0.4 | 2.12E−04 | 1.85E−03 |
| Serine protease 27 | PRSS27 | Q9BQR3 | −0.87 | −1.48 | −0.26 | 5.49E−03 | 3.02E−02 |
| Extracellular superoxide dismutase [Cu—Zn] | SOD3 | P08294 | −0.87 | −1.4 | −0.35 | 1.11E−03 | 7.82E−03 |
| Fc receptor-like protein 5 | FCRL5 | Q96RD9 | −0.87 | −1.38 | −0.36 | 7.92E−04 | 5.88E−03 |
| Protocadherin alpha-4 | PCDHA4 | Q9UN74 | −0.87 | −1.35 | −0.39 | 3.80E−04 | 3.07E−03 |
| Ectodysplasin-A, secreted form | EDA | Q92838 | −0.88 | −1.54 | −0.22 | 9.31E−03 | 4.72E−02 |
| Dual specificity mitogen-activated protein | MAP2K4 | P45985 | −0.89 | −1.57 | −0.22 | 9.30E−03 | 4.72E−02 |
| kinase kinase 4 | | | | | | | |
| Interleukin-36 receptor antagonist protein | IL36RN | Q9UBH0 | −0.9 | −1.43 | −0.36 | 9.79E−04 | 7.08E−03 |
| Cysteine-rich with EGF-like domain protein 1 | CRELD1 | Q96HD1 | −0.91 | −1.37 | −0.44 | 1.26E−04 | 1.18E−03 |
| Integrin alpha-V: beta-5 complex | ITGAV\|ITGB5 | P06756\|P18084 | −0.92 | −1.41 | −0.43 | 2.27E−04 | 1.95E−03 |
| Scavenger receptor class A member 5 | SCARA5 | Q6ZMJ2 | −0.93 | −1.5 | −0.36 | 1.50E−03 | 1.00E−02 |
| Tyrosine-protein kinase SYK: Protein kinase | SYK | P43405 | −0.93 | −1.64 | −0.23 | 9.61E−03 | 4.83E−02 |
| domain | | | | | | | |
| Cystatin-SN | CST1 | P01037 | −0.93 | −1.5 | −0.37 | 1.26E−03 | 8.70E−03 |
| Keratin, type II cytoskeletal 5 | KRT5 | P13647 | −0.94 | −1.44 | −0.44 | 2.28E−04 | 1.96E−03 |
| Pre-mRNA-processing factor 6: region 2 | PRPF6 | O94906 | −0.94 | −1.62 | −0.26 | 6.51E−03 | 3.48E−02 |
| Advanced glycosylation end product-specific | AGER | Q15109 | −0.95 | −1.23 | −0.67 | 3.63E−11 | 1.42E−09 |
| receptor, soluble | | | | | | | |
| Interleukin-27 subunit beta | EBI3 | Q14213 | −0.96 | −1.37 | −0.54 | 6.92E−06 | 8.88E−05 |
| Secretory phospholipase A2 receptor | PLA2R1 | Q13018 | −0.96 | −1.56 | −0.37 | 1.57E−03 | 1.04E−02 |
| CD209 antigen | CD209 | Q9NNX6 | −0.96 | −1.47 | −0.44 | 2.60E−04 | 2.20E−03 |
| Trypsin-1 | PRSS1 | P07477 | −0.96 | −1.47 | −0.44 | 3.19E−04 | 2.65E−03 |
| Interleukin-18-binding protein | IL18BP | O95998 | −0.96 | −1.58 | −0.34 | 2.55E−03 | 1.57E−02 |
| Tumor necrosis factor receptor superfamily | NGFR | P08138 | −0.96 | −1.42 | −0.5 | 3.72E−05 | 4.01E−04 |
| member 16 | | | | | | | |
| Heparan-sulfate 6-O-sulfotransferase 2 | HS6ST2 | Q96MM7 | −0.97 | −1.59 | −0.35 | 2.11E−03 | 1.35E−02 |
| MAM domain-containing | MDGA1 | Q8NFP4 | −0.98 | −1.56 | −0.39 | 1.01E−03 | 7.26E−03 |
| glycosylphosphatidylinositol anchor protein 1 | | | | | | | |
| Cystatin-SA | CST2 | P09228 | −0.98 | −1.35 | −0.61 | 2.44E−07 | 4.38E−06 |
| Semaphorin-6C | SEMA6C | Q9H3T2 | −0.98 | −1.64 | −0.32 | 3.57E−03 | 2.10E−02 |
| Chymotrypsin-like elastase family member 2A | CELA2A | P08217 | −0.99 | −1.54 | −0.45 | 3.65E−04 | 2.98E−03 |
| Diamine acetyltransferase 1 | SAT1 | P21673 | −1 | −1.72 | −0.28 | 6.79E−03 | 3.59E−02 |
| Alpha-amylase 2B | AMY2B | P19961 | −1.01 | −1.54 | −0.48 | 2.00E−04 | 1.75E−03 |
| Sex hormone-binding globulin | SHBG | P04278 | −1.01 | −1.34 | −0.68 | 2.08E−09 | 5.66E−08 |
| Lysophosphatidic acid phosphatase type 6 | ACP6 | Q9NPH0 | −1.01 | −1.58 | −0.43 | 6.27E−04 | 4.83E−03 |
| N-acetylated-alpha-linked acidic dipeptidase 2 | NAALAD2 | Q9Y3Q0 | −1.01 | −1.56 | −0.46 | 3.17E−04 | 2.63E−03 |
| Prostate-associated microseminoprotein | MSMP | Q1L6U9 | −1.01 | −1.53 | −0.5 | 1.00E−04 | 9.66E−04 |
| Leucine-rich repeat transmembrane protein | FLRT3 | Q9NZU0 | −1.02 | −1.74 | −0.31 | 4.98E−03 | 2.79E−02 |
| FLRT3: Extracellular domain | | | | | | | |
| Heterogeneous nuclear ribonucleoprotein H | HNRNPH1 | P31943 | −1.02 | −1.68 | −0.35 | 2.73E−03 | 1.66E−02 |
| Carbonic anhydrase 6 | CA6 | P23280 | −1.02 | −1.35 | −0.69 | 1.89E−09 | 5.16E−08 |
| Protein FAM3B | FAM3B | P58499 | −1.02 | −1.52 | −0.51 | 8.11E−05 | 8.07E−04 |
| PH and SEC7 domain-containing protein 2 | PSD2 | Q9BQI7 | −1.03 | −1.49 | −0.57 | 1.33E−05 | 1.60E−04 |
| PR domain zinc finger protein 4 | PRDM4 | Q9UKN5 | −1.05 | −1.82 | −0.27 | 8.22E−03 | 4.23E−02 |
| Alpha-taxilin | TXLNA | P40222 | −1.05 | −1.71 | −0.39 | 1.95E−03 | 1.27E−02 |
| Glutathione reductase, mitochondrial | GSR | P00390 | −1.05 | −1.73 | −0.38 | 2.30E−03 | 1.44E−02 |
| Drebrin-like protein | DBNL | Q9UJU6 | −1.05 | −1.79 | −0.31 | 5.34E−03 | 2.95E−02 |
| NAD-dependent protein deacetylase sirtuin-2 | SIRT2 | Q8IXJ6 | −1.05 | −1.69 | −0.41 | 1.23E−03 | 8.56E−03 |
| Sulfhydryl oxidase 2 | QSOX2 | Q6ZRP7 | −1.05 | −1.78 | −0.32 | 5.03E−03 | 2.82E−02 |
| Glypican-1 | GPC1 | P35052 | −1.05 | −1.84 | −0.26 | 9.23E−03 | 4.69E−02 |
| Oxygen-dependent coproporphyrinogen-III | CPOX | P36551 | −1.06 | −1.56 | −0.56 | 3.26E−05 | 3.61E−04 |
| oxidase, mitochondrial | | | | | | | |
| Glypican-5 | GPC5 | P78333 | −1.06 | −1.57 | −0.56 | 3.84E−05 | 4.11E−04 |
| Acidic mammalian chitinase | CHIA | Q9BZP6 | −1.07 | −1.87 | −0.27 | 8.79E−03 | 4.48E−02 |
| Type 2 lactosamine alpha-2,3-sialyltransferase | ST3GAL6 | Q9Y274 | −1.07 | −1.74 | −0.39 | 2.01E−03 | 1.30E−02 |
| Neuferricin | CYB5D2 | Q8WUJ1 | −1.08 | −1.86 | −0.3 | 6.48E−03 | 3.47E−02 |
| Insulin-like growth factor-binding protein 1 | IGFBP1 | P08833 | −1.08 | −1.32 | −0.84 | 1.56E−18 | 3.69E−16 |
| Cysteine-rich secretory protein 2 | CRISP2 | P16562 | −1.08 | −1.6 | −0.56 | 5.04E−05 | 5.26E−04 |
| Valine--tRNA ligase | VARS1 | P26640 | −1.09 | −1.91 | −0.26 | 1.00E−02 | 4.99E−02 |
| Tumor necrosis factor receptor type 1- | TRADD | Q15628 | −1.09 | −1.77 | −0.4 | 1.80E−03 | 1.18E−02 |
| associated DEATH domain protein | | | | | | | |
| Gastric intrinsic factor | CBLIF | P27352 | −1.1 | −1.56 | −0.65 | 2.03E−06 | 2.97E−05 |
| Apolipoprotein F | APOF | Q13790 | −1.11 | −1.53 | −0.68 | 3.66E−07 | 6.31E−06 |
| Phosphate-regulating neutral endopeptidase | PHEX | P78562 | −1.11 | −1.8 | −0.42 | 1.67E−03 | 1.10E−02 |
| Vacuolar protein sorting-associated protein 28 | VPS28 | Q9UK41 | −1.12 | −1.86 | −0.37 | 3.24E−03 | 1.94E−02 |
| homolog | | | | | | | |
| Cholecystokinin | CCK | P06307 | −1.12 | −1.96 | −0.28 | 8.86E−03 | 4.51E−02 |
| DNA repair protein RAD51 homolog 4 | RAD51D | O75771 | −1.13 | −1.76 | −0.51 | 3.46E−04 | 2.84E−03 |
| Galactoside 3(4)-L-fucosyltransferase | FUT3 | P21217 | −1.13 | −1.81 | −0.45 | 1.04E−03 | 7.44E−03 |
| Plexin-C1 | PLXNC1 | O60486 | −1.13 | −1.92 | −0.34 | 5.16E−03 | 2.87E−02 |
| Sodium channel subunit beta-2 | SCN2B | O60939 | −1.13 | −1.81 | −0.45 | 1.15E−03 | 8.04E−03 |
| Normal mucosa of esophagus-specific gene 1 | NMES1 | Q9C002 | −1.14 | −1.82 | −0.46 | 1.05E−03 | 7.45E−03 |
| protein | | | | | | | |
| Neuronal pentraxin-2 | NPTX2 | P47972 | −1.14 | −1.93 | −0.36 | 4.50E−03 | 2.57E−02 |
| Neurexophilin-2 | NXPH2 | O95156 | −1.15 | −1.54 | −0.75 | 1.30E−08 | 2.99E−07 |
| Retinol-binding protein 4 | RBP4 | P02753 | −1.16 | −1.92 | −0.41 | 2.57E−03 | 1.59E−02 |
| Dipeptidase 1 | DPEP1 | P16444 | −1.16 | −1.87 | −0.46 | 1.25E−03 | 8.67E−03 |
| Neuropilin-1 | NRP1 | O14786 | −1.17 | −2.04 | −0.3 | 8.60E−03 | 4.41E−02 |
| Glutamyl aminopeptidase | ENPEP | Q07075 | −1.18 | −1.82 | −0.54 | 3.29E−04 | 2.72E−03 |
| Disintegrin and metalloproteinase domain- | ADAM22 | Q9P0K1 | −1.19 | −1.75 | −0.62 | 3.75E−05 | 4.03E−04 |
| containing protein 22 | | | | | | | |
| Kit ligand | KITLG | P21583 | −1.19 | −1.97 | −0.41 | 2.72E−03 | 1.66E−02 |
| Pregnancy-specific beta-1-glycoprotein 3 | PSG3 | Q16557 | −1.2 | −1.76 | −0.63 | 3.26E−05 | 3.61E−04 |
| UDP-GlcNAc: betaGal beta-1,3-N- | B3GNT8 | Q7Z7M8 | −1.2 | −1.92 | −0.49 | 1.02E−03 | 7.31E−03 |
| acetylglucosaminyltransferase 8 | | | | | | | |
| Hexokinase-3 | HK3 | P52790 | −1.21 | −2.09 | −0.34 | 6.31E−03 | 3.39E−02 |
| fibromodulin | FMOD | Q06828 | −1.21 | −2.04 | −0.39 | 3.96E−03 | 2.28E−02 |
| Leukocyte immunoglobulin-like receptor | LILRA4 | P59901 | −1.21 | −1.95 | −0.46 | 1.49E−03 | 9.94E−03 |
| subfamily A member 4 | | | | | | | |
| Insulin-like growth factor-binding protein 2 | IGFBP2 | P18065 | −1.21 | −1.56 | −0.85 | 3.66E−11 | 1.42E−09 |
| Syntenin-1 | SDCBP | O00560 | −1.23 | −2.02 | −0.43 | 2.54E−03 | 1.57E−02 |
| EMILIN-3: region 1 | EMILIN3 | Q9NT22 | −1.23 | −1.89 | −0.57 | 2.68E−04 | 2.26E−03 |
| RNA polymerase II elongation factor ELL2 | ELL2 | O00472 | −1.24 | −2.05 | −0.42 | 2.96E−03 | 1.79E−02 |
| VPS10 domain-containing receptor SorCS1 | SORCS1 | Q8WY21 | −1.24 | −2.13 | −0.35 | 6.57E−03 | 3.51E−02 |
| Thrombospondin type-1 domain-containing | THSD1 | Q9NS62 | −1.24 | −2.14 | −0.34 | 6.66E−03 | 3.54E−02 |
| protein 1 | | | | | | | |
| Tomoregulin-1 | TMEFF1 | Q8IYR6 | −1.25 | −2.14 | −0.36 | 6.05E−03 | 3.27E−02 |
| Periostin | POSTN | Q15063 | −1.26 | −2.04 | −0.49 | 1.45E−03 | 9.72E−03 |
| Gastrokine-1 | GKN1 | Q9NS71 | −1.27 | −1.91 | −0.62 | 1.21E−04 | 1.14E−03 |
| Cerebellin-4 | CBLN4 | Q9NTU7 | −1.29 | −2 | −0.59 | 2.96E−04 | 2.48E−03 |
| EGF-like repeat and discoidin I-like domain- | EDIL3 | O43854 | −1.29 | −1.81 | −0.78 | 8.49E−07 | 1.33E−05 |
| containing protein 3 | | | | | | | |
| Lymphotoxin alpha1: beta2 | LTA\|LTB | P01374\|Q06643 | −1.3 | −2.16 | −0.43 | 3.42E−03 | 2.02E−02 |
| Delta and Notch-like epidermal growth factor- | DNER | Q8NFT8 | −1.3 | −1.98 | −0.63 | 1.62E−04 | 1.45E−03 |
| related receptor: Extracellular domain | | | | | | | |
| Low-density lipoprotein receptor-related | LRP4 | O75096 | −1.31 | −2.05 | −0.58 | 4.54E−04 | 3.61E−03 |
| protein 4 | | | | | | | |
| Iduronate 2-sulfatase | IDS | P22304 | −1.31 | −2.06 | −0.57 | 5.63E−04 | 4.39E−03 |
| Biglycan | BGN | P21810 | −1.31 | −2.11 | −0.52 | 1.22E−03 | 8.51E−03 |
| Chordin-like protein 1 | CHRDL1 | Q9BU40 | −1.31 | −2.2 | −0.42 | 3.95E−03 | 2.28E−02 |
| Secretogranin-1 | CHGB | P05060 | −1.31 | −2.14 | −0.49 | 1.72E−03 | 1.13E−02 |
| ADP-ribosylation factor-like protein 2 | ARL2 | P36404 | −1.32 | −1.93 | −0.72 | 2.10E−05 | 2.41E−04 |
| Pancreatic alpha-amylase | AMY2A | P04746 | −1.32 | −1.86 | −0.79 | 1.21E−06 | 1.84E−05 |
| Sonic hedgehog protein | SHH | Q15465 | −1.32 | −1.98 | −0.67 | 7.90E−05 | 7.90E−04 |
| Urokinase-type plasminogen activator | PLAU | P00749 | −1.32 | −2.21 | −0.43 | 3.68E−03 | 2.15E−02 |
| Asporin | ASPN | Q9BXN1 | −1.32 | −2.31 | −0.33 | 8.73E−03 | 4.46E−02 |
| Ras-related protein Rab-26 | RAB26 | Q9ULW5 | −1.32 | −2.1 | −0.54 | 9.09E−04 | 6.67E−03 |
| SLIT and NTRK-like protein 3 | SLITRK3 | O94933 | −1.33 | −1.69 | −0.97 | 5.56E−13 | 3.30E−11 |
| 72 kDa type IV collagenase | MMP2 | P08253 | −1.33 | −2.29 | −0.36 | 7.20E−03 | 3.78E−02 |
| Neurotrimin | NTM | Q9P121 | −1.33 | −2.2 | −0.46 | 2.65E−03 | 1.62E−02 |
| Interferon alpha/beta receptor 1 | IFNAR1 | P17181 | −1.33 | −2.12 | −0.54 | 1.01E−03 | 7.26E−03 |
| Prolow-density lipoprotein receptor-related | LRP1 | Q07954 | −1.34 | −2.2 | −0.47 | 2.40E−03 | 1.49E−02 |
| protein 1 | | | | | | | |
| Secreted frizzled-related protein 3 | FRZB | Q92765 | −1.34 | −2.02 | −0.65 | 1.39E−04 | 1.28E−03 |
| Proteoglycan 3 | PRG3 | Q9Y2Y8 | −1.35 | −1.85 | −0.85 | 1.12E−07 | 2.16E−06 |
| Sclerostin | SOST | Q9BQB4 | −1.36 | −2.3 | −0.42 | 4.76E−03 | 2.70E−02 |
| Glycine N-methyltransferase | GNMT | Q14749 | −1.36 | −2.24 | −0.49 | 2.31E−03 | 1.45E−02 |
| Short-chain specific acyl-CoA dehydrogenase, | ACADS | P16219 | −1.36 | −2.24 | −0.47 | 2.60E−03 | 1.60E−02 |
| mitochondrial | | | | | | | |
| Heme oxygenase 1 | HMOX1 | P09601 | −1.36 | −2.07 | −0.66 | 1.62E−04 | 1.45E−03 |
| Programmed cell death 1 ligand 2 | PDCD1LG2 | Q9BQ51 | −1.36 | −2.27 | −0.45 | 3.33E−03 | 1.98E−02 |
| Neurotrophin-3 | NTF3 | P20783 | −1.36 | −2.24 | −0.48 | 2.46E−03 | 1.53E−02 |
| Trypsin-3 | PRSS3 | P35030 | −1.37 | −1.98 | −0.76 | 1.03E−05 | 1.27E−04 |
| Mast/stem cell growth factor receptor Kit | KIT | P10721 | −1.37 | −2.09 | −0.66 | 1.67E−04 | 1.48E−03 |
| Cartilage acidic protein 1 | CRTAC1 | Q9NQ79 | −1.37 | −2.05 | −0.69 | 8.79E−05 | 8.62E−04 |
| Cerebellin-1 | CBLN1 | P23435 | −1.37 | −1.81 | −0.92 | 2.54E−09 | 6.68E−08 |
| 3-mercaptopyruvate sulfurtransferase | MPST | P25325 | −1.38 | −2.15 | −0.61 | 4.43E−04 | 3.54E−03 |
| Dickkopf-like protein 1 | DKKL1 | Q9UK85 | −1.38 | −2.21 | −0.54 | 1.27E−03 | 8.72E−03 |
| Retinoic acid early transcript 1G protein | RAET1G | Q6H3X3 | −1.38 | −2.39 | −0.37 | 7.21E−03 | 3.78E−02 |
| Leucine-rich repeat-containing protein 32 | LRRC32 | Q14392 | −1.38 | −2.31 | −0.46 | 3.46E−03 | 2.04E−02 |
| Transmembrane glycoprotein NMB: | GPNMB | Q14956 | −1.38 | −2.18 | −0.58 | 7.14E−04 | 5.37E−03 |
| Cytoplasmic domain | | | | | | | |
| Carnitine O-palmitoyltransferase 1, muscle | CPT1B | Q92523 | −1.39 | −2.34 | −0.45 | 3.87E−03 | 2.25E−02 |
| isoform | | | | | | | |
| Carbonic anhydrase-related protein 11 | CA11 | O75493 | −1.39 | −2.11 | −0.67 | 1.49E−04 | 1.36E−03 |
| Protein delta homolog 2 | DLK2 | Q6UY11 | −1.39 | −2.09 | −0.69 | 9.49E−05 | 9.25E−04 |
| Protein jagged-1: Extracellular domain | JAG1 | P78504 | −1.4 | −2.32 | −0.48 | 2.77E−03 | 1.69E−02 |
| NADH-cytochrome b5 reductase 3 | CYB5R3 | P00387 | −1.4 | −1.83 | −0.97 | 2.55E−10 | 8.07E−09 |
| Alpha-N-acetylgalactosaminide alpha-2,6- | ST6GALNAC6 | Q969X2 | −1.41 | −2.39 | −0.44 | 4.46E−03 | 2.55E−02 |
| sialyltransferase 6 | | | | | | | |
| Monocyte differentiation antigen CD14 | CD14 | P08571 | −1.41 | −2.46 | −0.36 | 8.54E−03 | 4.38E−02 |
| Arfaptin-2 | ARFIP2 | P53365 | −1.42 | −2.16 | −0.68 | 1.62E−04 | 1.45E−03 |
| Histone H3.1 | H3C1 | P68431 | −1.42 | −2.27 | −0.58 | 1.00E−03 | 7.21E−03 |
| Insulin-like growth factor-binding protein 5 | IGFBP5 | P24593 | −1.42 | −2.12 | −0.72 | 7.21E−05 | 7.28E−04 |
| Netrin-G1 | NTNG1 | Q9Y2I2 | −1.42 | −2.25 | −0.59 | 7.71E−04 | 5.75E−03 |
| CMP-N-acetylneuraminate-beta-galactosamide- | ST3GAL1 | Q11201 | −1.42 | −2.09 | −0.75 | 3.38E−05 | 3.72E−04 |
| alpha-2,3-sialyltransferase 1 | | | | | | | |
| Chromogranin-A | CHGA | P10645 | −1.42 | −2.21 | −0.63 | 4.15E−04 | 3.33E−03 |
| Prosaposin | PSAP | P07602 | −1.43 | −2.3 | −0.56 | 1.33E−03 | 9.07E−03 |
| NAD-dependent protein deacetylase sirtuin-3, | SIRT3 | Q9NTG7 | −1.44 | −2.23 | −0.65 | 3.36E−04 | 2.77E−03 |
| mitochondrial | | | | | | | |
| Laminin-2 | LAMA2\|LAMB1\|LAMC1 | P24043\|P07942\|P11047 | −1.44 | −2.47 | −0.42 | 5.95E−03 | 3.24E−02 |
| Islet amyloid polypeptide | IAPP | P10997 | −1.44 | −2.13 | −0.75 | 4.02E−05 | 4.27E−04 |
| Cathepsin B | CTSB | P07858 | −1.46 | −2.05 | −0.87 | 1.20E−06 | 1.84E−05 |
| Neuronal pentraxin-1 | NPTX1 | Q15818 | −1.46 | −2.15 | −0.76 | 3.95E−05 | 4.21E−04 |
| Contactin-2 | CNTN2 | Q02246 | −1.47 | −2.12 | −0.82 | 9.22E−06 | 1.15E−04 |
| Plexin domain-containing protein 1 | PLXDC1 | Q8IUK5 | −1.47 | −2.31 | −0.64 | 5.52E−04 | 4.31E−03 |
| Neuroendocrine convertase 1 | PCSK1 | P29120 | −1.48 | −2.31 | −0.65 | 4.68E−04 | 3.69E−03 |
| Ceruloplasmin | CP | P00450 | −1.48 | −2.26 | −0.71 | 1.75E−04 | 1.55E−03 |
| Complement component C7 | C7 | P10643 | −1.48 | −2.61 | −0.35 | 9.97E−03 | 4.98E−02 |
| C-C motif chemokine 23 | CCL23 | P55773 | −1.48 | −2.15 | −0.81 | 1.39E−05 | 1.66E−04 |
| Fibroblast growth factor receptor 3: | FGFR3 | P22607 | −1.49 | −2.23 | −0.74 | 9.75E−05 | 9.46E−04 |
| Extracellular domain | | | | | | | |
| Complexin-2 | CPLX2 | Q6PUV4 | −1.49 | −2.13 | −0.84 | 6.74E−06 | 8.67E−05 |
| Collectin-10 | COLEC10 | Q9Y6Z7 | −1.49 | −2.57 | −0.4 | 7.24E−03 | 3.79E−02 |
| Transcription initiation factor TFIID subunit 10 | TAF10 | Q12962 | −1.5 | −2.32 | −0.67 | 3.65E−04 | 2.98E−03 |
| Peroxisome assembly protein 26 | PEX26 | Q7Z412 | −1.5 | −2.35 | −0.65 | 5.71E−04 | 4.44E−03 |
| Semaphorin-7A | SEMA7A | O75326 | −1.5 | −2.33 | −0.66 | 4.48E−04 | 3.58E−03 |
| Proactivator polypeptide-like 1 | PSAPL1 | Q6NUJ1 | −1.5 | −2.08 | −0.91 | 5.08E−07 | 8.46E−06 |
| Leucine-rich repeat and transmembrane | LRTM2 | Q8N967 | −1.5 | −2.51 | −0.49 | 3.64E−03 | 2.14E−02 |
| domain-containing protein 2 | | | | | | | |
| Polypeptide N-acetylgalactosaminyltransferase | GALNT13 | Q8IUC8 | −1.51 | −2.23 | −0.8 | 3.63E−05 | 3.94E−04 |
| 13 | | | | | | | |
| TAR DNA-binding protein 43 | TARDBP | Q13148 | −1.51 | −2.52 | −0.51 | 3.22E−03 | 1.93E−02 |
| Uncharacterized protein C3orf18 | C3orf18 | Q9UK00 | −1.51 | −2.63 | −0.39 | 8.12E−03 | 4.19E−02 |
| Slit homolog 2 protein | SLIT2 | O94813 | −1.53 | −2.39 | −0.67 | 4.85E−04 | 3.81E−03 |
| Cathepsin E | CTSE | P14091 | −1.55 | −2.35 | −0.75 | 1.38E−04 | 1.28E−03 |
| G2/mitotic-specific cyclin-B2 | CCNB2 | O95067 | −1.55 | −2.53 | −0.56 | 2.10E−03 | 1.34E−02 |
| Gelsolin | GSN | P06396 | −1.55 | −2.62 | −0.47 | 4.93E−03 | 2.77E−02 |
| Kidney-associated antigen 1 | KAAG1 | Q9UBP8 | −1.56 | −2.51 | −0.61 | 1.27E−03 | 8.73E−03 |
| Protein MENT | MENT | Q9BUN1 | −1.56 | −2.12 | −0.99 | 6.62E−08 | 1.35E−06 |
| Histone deacetylase 8 | HDAC8 | Q9BY41 | −1.57 | −2.75 | −0.38 | 9.55E−03 | 4.81E−02 |
| Neuronal cell adhesion molecule | NRCAM | Q92823 | −1.57 | −2.51 | −0.63 | 1.02E−03 | 7.31E−03 |
| Deoxycytidine kinase | DCK | P27707 | −1.57 | −2.77 | −0.37 | 1.01E−02 | 5.00E−02 |
| Carboxypeptidase E | CPE | P16870 | −1.58 | −2.62 | −0.55 | 2.65E−03 | 1.62E−02 |
| Arylsulfatase K | ARSK | Q6UWY0 | −1.58 | −2.66 | −0.49 | 4.30E−03 | 2.46E−02 |
| PILR alpha-associated neural protein | PIANP | Q8IYJ0 | −1.58 | −2.25 | −0.9 | 4.75E−06 | 6.32E−05 |
| Adiponectin | ADIPOQ | Q15848 | −1.59 | −2.01 | −1.17 | 1.65E−13 | 1.17E−11 |
| Insulin-like growth factor 1 receptor | IGF1R | P08069 | −1.59 | −2.6 | −0.57 | 2.21E−03 | 1.40E−02 |
| Complement C1q tumor necrosis factor-related | C1QTNF3 | Q9BXJ4 | −1.6 | −2.6 | −0.6 | 1.75E−03 | 1.15E−02 |
| protein 3 | | | | | | | |
| Hematopoietic prostaglandin D synthase | HPGDS | O60760 | −1.61 | −2.28 | −0.94 | 2.62E−06 | 3.70E−05 |
| Tenascin | TNC | P24821 | −1.61 | −2.55 | −0.67 | 7.77E−04 | 5.78E−03 |
| Agouti-related protein | AGRP | O00253 | −1.62 | −2.34 | −0.91 | 8.33E−06 | 1.06E−04 |
| Cyclin-dependent kinase 8: Cyclin-C complex | CDK8\|CCNC | P49336\|P24863 | −1.62 | −2.57 | −0.66 | 9.30E−04 | 6.78E−03 |
| Alpha-1,3-mannosyl-glycoprotein 4-beta-N- | MGAT4B | Q9UQ53 | −1.64 | −2.46 | −0.83 | 8.09E−05 | 8.07E−04 |
| acetylglucosaminyltransferase B | | | | | | | |
| Acidic leucine-rich nuclear phosphoprotein | ANP32A | P39687 | −1.65 | −2.63 | −0.67 | 1.02E−03 | 7.31E−03 |
| 32 family member A | | | | | | | |
| Dihydrolipoyl dehydrogenase, mitochondrial | DLD | P09622 | −1.65 | −2.37 | −0.93 | 7.00E−06 | 8.95E−05 |
| Metalloproteinase inhibitor 2 | TIMP2 | P16035 | −1.65 | −2.79 | −0.5 | 4.85E−03 | 2.73E−02 |
| tRNA (guanine-N(7)-)-methyltransferase | METTL1 | Q9UBP6 | −1.66 | −2.72 | −0.59 | 2.35E−03 | 1.47E−02 |
| Ephrin-A3 | EFNA3 | P52797 | −1.66 | −2.51 | −0.81 | 1.30E−04 | 1.21E−03 |
| Ectonucleotide pyrophosphatase/ | ENPP5 | Q9UJA9 | −1.67 | −2.32 | −1.03 | 3.87E−07 | 6.64E−06 |
| phosphodiesterase family member 5 | | | | | | | |
| Inositol polyphosphate 5-phosphatase OCRL-1 | OCRL | Q01968 | −1.69 | −2.6 | −0.78 | 2.80E−04 | 2.35E−03 |
| Complexin-1 | CPLX1 | O14810 | −1.69 | −2.51 | −0.87 | 5.24E−05 | 5.44E−04 |
| Tumor necrosis factor ligand superfamily | TNFSF12 | O43508 | −1.69 | −2.43 | −0.95 | 8.94E−06 | 1.12E−04 |
| member 12 | | | | | | | |
| Appetite-regulating hormone | GHRL | Q9UBU3 | −1.69 | −2.52 | −0.85 | 8.39E−05 | 8.31E−04 |
| Sodium-coupled monocarboxylate transporter 1 | SLC5A8 | Q8N695 | −1.7 | −2.47 | −0.93 | 1.45E−05 | 1.73E−04 |
| Myeloid zinc finger 1 | MZF1 | P28698 | −1.7 | −2.56 | −0.85 | 9.54E−05 | 9.28E−04 |
| Reticulocalbin-3 | RCN3 | Q96D15 | −1.7 | −2.57 | −0.84 | 1.18E−04 | 1.12E−03 |
| Protein kinase C-binding protein NELL1 | NELL1 | Q92832 | −1.7 | −2.27 | −1.12 | 9.29E−09 | 2.20E−07 |
| Pikachurin | EGFLAM | Q63HQ2 | −1.71 | −2.59 | −0.84 | 1.19E−04 | 1.13E−03 |
| Intelectin-1 | ITLN1 | Q8WWA0 | −1.73 | −2.59 | −0.88 | 7.49E−05 | 7.53E−04 |
| Endothelial cell-selective adhesion molecule | ESAM | Q96AP7 | −1.73 | −2.65 | −0.81 | 2.30E−04 | 1.97E−03 |
| Alpha-hemoglobin-stabilizing protein | AHSP | Q9NZD4 | −1.73 | −2.56 | −0.89 | 4.84E−05 | 5.07E−04 |
| Cell adhesion molecule 1 | CADM1 | Q9BY67 | −1.74 | −2.65 | −0.84 | 1.60E−04 | 1.44E−03 |
| Cyclin-dependent kinase 4 inhibitor C | CDKN2C | P42773 | −1.75 | −2.81 | −0.7 | 1.12E−03 | 7.86E−03 |
| Sodium/potassium-transporting ATPase subunit | ATP1B1 | P05026 | −1.75 | −2.29 | −1.22 | 1.68E−10 | 5.68E−09 |
| beta-1 | | | | | | | |
| Matrilin-3 | MATN3 | O15232 | −1.75 | −2.43 | −1.07 | 5.42E−07 | 8.96E−06 |
| Phospholipid transfer protein | PLTP | P55058 | −1.76 | −2.74 | −0.79 | 3.88E−04 | 3.13E−03 |
| Opioid-binding protein/cell adhesion molecule | OPCML | Q14982 | −1.76 | −2.53 | −0.99 | 7.66E−06 | 9.78E−05 |
| SLIT and NTRK-like protein 5 | SLITRK5 | O94991 | −1.76 | −2.45 | −1.07 | 6.23E−07 | 1.00E−05 |
| Insulin-like growth factor I | IGF1 | P05019 | −1.77 | −2.5 | −1.04 | 2.11E−06 | 3.05E−05 |
| Uncharacterized protein C11orf87 | C11orf87 | Q6NUJ2 | −1.78 | −3.07 | −0.5 | 6.65E−03 | 3.54E−02 |
| Signal transducer and activator of | STAT5B | P51692 | −1.8 | −2.95 | −0.65 | 2.12E−03 | 1.35E−02 |
| transcription 5B | | | | | | | |
| Serine protease inhibitor Kazal-type 5 | SPINK5 | Q9NQ38 | −1.8 | −2.65 | −0.96 | 2.84E−05 | 3.20E−04 |
| Apolipoprotein A-IV | APOA4 | P06727 | −1.81 | −2.57 | −1.04 | 3.47E−06 | 4.76E−05 |
| Cellular tumor antigen p53 R175H mutant | TP53 | P04637 | −1.81 | −2.92 | −0.69 | 1.46E−03 | 9.80E−03 |
| Amyloid-like protein 1 | APLP1 | P51693 | −1.81 | −2.29 | −1.33 | 1.65E−13 | 1.17E−11 |
| Testican-2 | SPOCK2 | Q92563 | −1.82 | −2.67 | −0.97 | 2.90E−05 | 3.25E−04 |
| Hippocalcin-like protein 1 | HPCAL1 | P37235 | −1.83 | −2.75 | −0.92 | 8.85E−05 | 8.66E−04 |
| Receptor tyrosine-protein kinase erbB-3 | ERBB3 | P21860 | −1.83 | −2.82 | −0.85 | 2.71E−04 | 2.28E−03 |
| Peflin | PEF1 | Q9UBV8 | −1.84 | −2.59 | −1.08 | 1.95E−06 | 2.86E−05 |
| von Willebrand factor A domain-containing | VWA2 | Q5GFL6 | −1.84 | −2.52 | −1.16 | 1.12E−07 | 2.16E−06 |
| protein 2 | | | | | | | |
| Chitinase-3-like protein 2 | CHI3L2 | Q15782 | −1.84 | −2.94 | −0.73 | 1.17E−03 | 8.14E−03 |
| Ck-beta-8-1 | CCL23 | P55773 | −1.85 | −2.38 | −1.31 | 1.82E−11 | 7.83E−10 |
| Glutaminyl-peptide cyclotransferase-like | QPCTL | Q9NXS2 | −1.85 | −2.64 | −1.05 | 5.04E−06 | 6.64E−05 |
| protein | | | | | | | |
| Oligodendrocyte-myelin glycoprotein | OMG | P23515 | −1.86 | −2.35 | −1.38 | 5.17E−14 | 3.96E−12 |
| Transforming growth factor beta receptor | TGFBR3 | Q03167 | −1.86 | −2.87 | −0.85 | 3.19E−04 | 2.65E−03 |
| type 3 | | | | | | | |
| Neurexin-3-beta | NRXN3 | Q9HDB5 | −1.86 | −2.66 | −1.06 | 5.97E−06 | 7.74E−05 |
| Carbonic anhydrase-related protein 10 | CA10 | Q9NS85 | −1.88 | −2.59 | −1.17 | 2.17E−07 | 3.93E−06 |
| Cq-related factor | C1QL1 | O75973 | −1.88 | −2.87 | −0.9 | 1.78E−04 | 1.57E−03 |
| Alcohol dehydrogenase class 4 mu/sigma chain | ADH7 | P40394 | −1.89 | −2.52 | −1.25 | 5.85E−09 | 1.45E−07 |
| Prenylcysteine oxidase 1 | PCYOX1 | Q9UHG3 | −1.89 | −2.63 | −1.14 | 7.84E−07 | 1.24E−05 |
| Interleukin-6 receptor subunit alpha | IL6R | P08887 | −1.91 | −2.67 | −1.15 | 8.02E−07 | 1.26E−05 |
| Interleukin-1 Receptor accessory protein | IL1RAP | Q9NPH3 | −1.93 | −2.51 | −1.35 | 6.97E−11 | 2.51E−09 |
| Collectin-12 | COLEC12 | Q5KU26 | −1.93 | −2.57 | −1.28 | 4.91E−09 | 1.23E−07 |
| Bile acid receptor | NR1H4 | Q96RI1 | −1.93 | −3.22 | −0.64 | 3.35E−03 | 1.99E−02 |
| Aromatic-L-amino-acid decarboxylase | DDC | P20711 | −1.94 | −2.85 | −1.03 | 3.28E−05 | 3.62E−04 |
| Pregnancy-specific beta-1-glycoprotein 5 | PSG5 | Q15238 | −1.94 | −2.61 | −1.28 | 9.30E−09 | 2.20E−07 |
| Neuronal growth regulator 1 | NEGR1 | Q7Z3B1 | −1.97 | −3.3 | −0.63 | 3.89E−03 | 2.25E−02 |
| MARCKS-related protein | MARCKSL1 | P49006 | −1.98 | −3.25 | −0.7 | 2.35E−03 | 1.47E−02 |
| Repulsive guidance molecule A | RGMA | Q96B86 | −1.98 | −3.12 | −0.85 | 6.05E−04 | 4.68E−03 |
| 26S proteasome non-ATPase regulatory | PSMD10 | O75832 | −2 | −3.19 | −0.81 | 9.84E−04 | 7.10E−03 |
| subunit 10 | | | | | | | |
| Intercellular adhesion molecule 5 | ICAM5 | Q9UMF0 | −2 | −2.61 | −1.39 | 1.34E−10 | 4.62E−09 |
| P-selectin glycoprotein ligand 1: Extracellular | SELPLG | Q14242 | −2.01 | −2.66 | −1.36 | 1.85E−09 | 5.12E−08 |
| domain | | | | | | | |
| cGMP-dependent 3′,5′-cyclic | PDE2A | O00408 | −2.03 | −3.04 | −1.03 | 7.43E−05 | 7.49E−04 |
| phosphodiesterase | | | | | | | |
| Aggrecan core protein | ACAN | P16112 | −2.04 | −2.97 | −1.12 | 1.48E−05 | 1.76E−04 |
| Alpha-(1,3)-fucosyltransferase 10 | FUT10 | Q6P4F1 | −2.04 | −2.83 | −1.25 | 4.61E−07 | 7.75E−06 |
| Insulin-like growth factor-binding protein 3 | IGFBP3 | P17936 | −2.05 | −2.96 | −1.15 | 9.40E−06 | 1.17E−04 |
| BDNF/NT-3 growth factors receptor | NTRK2 | Q16620 | −2.05 | −3 | −1.09 | 2.67E−05 | 3.04E−04 |
| Intercellular adhesion molecule 5 | ICAM5 | Q9UMF0 | −2.05 | −2.68 | −1.43 | 1.76E−10 | 5.88E−09 |
| Leucine-rich repeat-containing protein 4C | LRRC4C | Q9HCJ2 | −2.06 | −3.1 | −1.02 | 1.03E−04 | 9.89E−04 |
| TOM1-like protein 1 | TOM1L1 | O75674 | −2.07 | −3.31 | −0.82 | 1.15E−03 | 8.03E−03 |
| Homeodomain-interacting protein kinase 3 | HIPK3 | Q9H422 | −2.07 | −3.26 | −0.88 | 6.76E−04 | 5.17E−03 |
| Zinc finger protein 334 | ZNF334 | Q9HCZ1 | −2.08 | −3.17 | −0.99 | 1.93E−04 | 1.69E−03 |
| Neurexin-3 | NRXN3 | Q9Y4C0 | −2.11 | −2.9 | −1.32 | 1.57E−07 | 2.94E−06 |
| Glypican-3 | GPC3 | P51654 | −2.11 | −2.65 | −1.58 | 1.20E−14 | 1.05E−12 |
| Brevican core protein | BCAN | Q96GW7 | −2.12 | −2.92 | −1.32 | 1.90E−07 | 3.48E−06 |
| Platelet-activating factor acetylhydrolase | PLA2G7 | Q13093 | −2.12 | −2.73 | −1.51 | 1.20E−11 | 5.44E−10 |
| N-acetyllactosaminide beta-1,3-N- | B3GNT2 | Q9NY97 | −2.12 | −3.37 | −0.86 | 9.66E−04 | 6.99E−03 |
| acetylglucosaminyltransferase 2 | | | | | | | |
| Amphoterin-induced protein 2 | AMIGO2 | Q86SJ2 | −2.13 | −3.1 | −1.17 | 1.52E−05 | 1.79E−04 |
| Tubulointerstitial nephritis antigen-like | TINAGL1 | Q9GZM7 | −2.14 | −2.96 | −1.32 | 3.02E−07 | 5.34E−06 |
| Neurotrimin | NTM | Q9P121 | −2.15 | −2.92 | −1.37 | 5.65E−08 | 1.16E−06 |
| Interleukin-1 receptor type 2 | IL1R2 | P27930 | −2.15 | −3.03 | −1.27 | 1.72E−06 | 2.56E−05 |
| Immunoglobulin superfamily DCC subclass | IGDCC4 | Q8TDY8 | −2.15 | −3 | −1.31 | 6.52E−07 | 1.05E−05 |
| member 4 | | | | | | | |
| Limbic system-associated membrane protein | LSAMP | Q13449 | −2.16 | −3.2 | −1.13 | 4.19E−05 | 4.43E−04 |
| Fibroblast growth factor 9 | FGF9 | P31371 | −2.17 | −3.22 | −1.12 | 4.86E−05 | 5.08E−04 |
| Dual specificity protein phosphatase 13 | DUSP13 | Q6B8I1 | −2.17 | −2.81 | −1.53 | 2.66E−11 | 1.09E−09 |
| isoform A | | | | | | | |
| Golgi-associated plant pathogenesis-related | GLIPR2 | Q9H4G4 | −2.18 | −3.22 | −1.15 | 3.54E−05 | 3.86E−04 |
| protein 1 | | | | | | | |
| ER membrane protein complex subunit 4 | EMC4 | Q5J8M3 | −2.19 | −3.1 | −1.29 | 2.07E−06 | 3.02E−05 |
| Proteasome subunit beta type-9 | PSMB9 | P28065 | −2.19 | −3.14 | −1.23 | 7.83E−06 | 9.97E−05 |
| Angiopoietin-1 receptor, soluble | TEK | Q02763 | −2.19 | −3.21 | −1.17 | 2.74E−05 | 3.10E−04 |
| Beta-1,4-galactosyltransferase 6 | B4GALT6 | Q9UBX8 | −2.2 | −2.97 | −1.42 | 3.15E−08 | 6.71E−07 |
| Inositol monophosphatase 3 | BPNT2 | Q9NX62 | −2.2 | −2.99 | −1.42 | 3.90E−08 | 8.23E−07 |
| L-Selectin | SELL | P14151 | −2.21 | −3.3 | −1.12 | 7.04E−05 | 7.13E−04 |
| Disintegrin and metalloproteinase domain- | ADAM23 | O75077 | −2.21 | −2.96 | −1.45 | 1.18E−08 | 2.74E−07 |
| containing protein 23 | | | | | | | |
| cGMP-dependent protein kinase 1, beta | PRKG1 | Q13976 | −2.22 | −3.92 | −0.53 | 1.00E−02 | 4.99E−02 |
| isozyme | | | | | | | |
| UPF0577 protein KIAA1324: Extracellular | ELAPOR1 | Q6UXG2 | −2.23 | −3.16 | −1.31 | 2.26E−06 | 3.27E−05 |
| domain | | | | | | | |
| RGM domain family member B | RGMB | Q6NW40 | −2.23 | −3.32 | −1.13 | 7.01E−05 | 7.13E−04 |
| Uronyl 2-sulfotransferase | UST | Q9Y2C2 | −2.23 | −3.13 | −1.33 | 1.30E−06 | 1.95E−05 |
| A disintegrin and metalloproteinase with | ADAMTS13 | Q76LX8 | −2.27 | −3.02 | −1.52 | 2.84E−09 | 7.36E−08 |
| thrombospondin motifs 13 | | | | | | | |
| Inactive tyrosine-protein kinase transmembrane | ROR1 | Q01973 | −2.28 | −3.08 | −1.49 | 2.10E−08 | 4.61E−07 |
| receptor ROR1 | | | | | | | |
| Cartilage intermediate layer protein 2 | CILP2 | Q8IUL8 | −2.29 | −2.98 | −1.6 | 9.07E−11 | 3.20E−09 |
| Scavenger receptor class F member 2: | SCARF2 | Q96GP6 | −2.31 | −3.37 | −1.25 | 1.93E−05 | 2.24E−04 |
| Extracellular domain | | | | | | | |
| Group XIIB secretory phospholipase A2-like | PLA2G12B | Q9BX93 | −2.32 | −3.04 | −1.61 | 2.33E−10 | 7.50E−09 |
| protein | | | | | | | |
| Hedgehog-interacting protein | HHIP | Q96QV1 | −2.33 | −3.3 | −1.36 | 2.81E−06 | 3.96E−05 |
| Zinc finger protein 18 | ZNF18 | P17022 | −2.35 | −3.53 | −1.17 | 9.99E−05 | 9.66E−04 |
| PILR alpha-associated neural protein | PIANP | Q8IYJ0 | −2.35 | −3.04 | −1.65 | 4.15E−11 | 1.57E−09 |
| Fibroblast growth factor receptor 1 | FGFR1 | P11362 | −2.35 | −3.44 | −1.26 | 2.59E−05 | 2.95E−04 |
| Transmembrane protein 132C: Extracellular | TMEM132C | Q8N3T6 | −2.35 | −3.05 | −1.66 | 3.42E−11 | 1.36E−09 |
| domain | | | | | | | |
| 14-3-3 protein epsilon | YWHAE | P62258 | −2.37 | −3.37 | −1.38 | 3.08E−06 | 4.28E−05 |
| WAP, Kazal, immunoglobulin, Kunitz and | WFIKKN2 | Q8TEU8 | −2.37 | −3.1 | −1.64 | 2.75E−10 | 8.66E−09 |
| NTR | | | | | | | |
| domain-containing protein 2 | | | | | | | |
| Immunoglobulin superfamily containing | ISLR2 | Q6UXK2 | −2.38 | −3.31 | −1.46 | 4.53E−07 | 7.65E−06 |
| leucine-rich repeat protein 2 | | | | | | | |
| Interleukin-6 receptor subunit beta | IL6ST | P40189 | −2.39 | −3.73 | −1.06 | 4.51E−04 | 3.60E−03 |
| Interleukin-1 receptor type 1 | IL1R1 | P14778 | −2.4 | −3.16 | −1.63 | 8.25E−10 | 2.43E−08 |
| Endoglin | ENG | P17813 | −2.4 | −3.3 | −1.49 | 2.05E−07 | 3.75E−06 |
| Insulin-like growth factor-binding protein | IGFALS | P35858 | −2.4 | −3.32 | −1.47 | 4.06E−07 | 6.88E−06 |
| complex acid labile subunit | | | | | | | |
| Noncompact myelin-associated protein | NCMAP | Q5T1S8 | −2.42 | −3.33 | −1.5 | 2.59E−07 | 4.64E−06 |
| Stromal cell-derived factor 1 | CXCL12 | P48061 | −2.42 | −3.36 | −1.47 | 5.51E−07 | 9.02E−06 |
| Transmembrane protein 132A | TMEM132A | Q24JP5 | −2.42 | −3.12 | −1.72 | 1.24E−11 | 5.54E−10 |
| Protocadherin-10: Extracellular domain | PCDH10 | Q9P2E7 | −2.44 | −3.17 | −1.71 | 6.87E−11 | 2.50E−09 |
| Hepatocyte growth factor receptor | MET | P08581 | −2.45 | −3.42 | −1.49 | 6.16E−07 | 9.95E−06 |
| GDNF family receptor alpha-2 | GFRA2 | O00451 | −2.47 | −3.52 | −1.41 | 4.89E−06 | 6.48E−05 |
| Desmoglein-2 | DSG2 | Q14126 | −2.47 | −3.32 | −1.61 | 1.63E−08 | 3.69E−07 |
| Superoxide dismutase [Mn], mitochondrial | SOD2 | P04179 | −2.48 | −3.33 | −1.63 | 1.17E−08 | 2.73E−07 |
| Transmembrane protein 132D | TMEM132D | Q14C87 | −2.51 | −3.17 | −1.84 | 2.02E−13 | 1.41E−11 |
| Bifunctional heparan sulfate N-deacetylase/N- | NDST1 | P52848 | −2.51 | −3.83 | −1.19 | 1.92E−04 | 1.69E−03 |
| sulfotransferase 1 | | | | | | | |
| Receptor-type tyrosine-protein phosphatase S | PTPRS | Q13332 | −2.52 | −3.63 | −1.41 | 8.65E−06 | 1.09E−04 |
| Tumor necrosis factor-inducible gene 6 protein | TNFAIP6 | P98066 | −2.53 | −3.13 | −1.94 | 7.05E−17 | 1.06E−14 |
| Semaphorin-3G | SEMA3G | Q9NS98 | −2.55 | −3.57 | −1.53 | 1.05E−06 | 1.63E−05 |
| Mannosyl-oligosaccharide 1,2-alpha- | MAN1C1 | Q9NR34 | −2.56 | −3.88 | −1.25 | 1.38E−04 | 1.27E−03 |
| mannosidase IC | | | | | | | |
| SLIT and NTRK-like protein 1 | SLITRK1 | Q96PX8 | −2.56 | −3.15 | −1.97 | 2.03E−17 | 3.60E−15 |
| Glucoside xylosyltransferase 1 | GXYLT1 | Q4G148 | −2.57 | −3.34 | −1.8 | 6.55E−11 | 2.41E−09 |
| Thrombin | F2 | P00734 | −2.58 | −3.35 | −1.81 | 7.07E−11 | 2.53E−09 |
| Alpha-1-antichymotrypsin | SERPINA3 | P01011 | −2.59 | −4.48 | −0.7 | 7.17E−03 | 3.76E−02 |
| S-phase kinase-associated protein 1 | SKP1 | P63208 | −2.59 | −4.23 | −0.95 | 2.02E−03 | 1.30E−02 |
| Growth/differentiation factor 2 | GDF2 | Q9UK05 | −2.59 | −3.5 | −1.68 | 2.52E−08 | 5.43E−07 |
| Ecto-ADP-ribosyltransferase 3 | ART3 | Q13508 | −2.59 | −3.22 | −1.97 | 6.50E−16 | 7.52E−14 |
| Indian hedgehog protein | IHH | Q14623 | −2.6 | −3.46 | −1.75 | 2.86E−09 | 7.39E−08 |
| Histone-lysine N-methyltransferase EHMT2 | EHMT2 | Q96KQ7 | −2.6 | −3.14 | −2.07 | 1.99E−21 | 8.99E−19 |
| Dickkopf-related protein 3 | DKK3 | Q9UBP4 | −2.62 | −3.47 | −1.76 | 2.72E−09 | 7.13E−08 |
| Neuronal pentraxin receptor | NPTXR | O95502 | −2.63 | −3.21 | −2.05 | 8.50E−19 | 2.17E−16 |
| Endothelial cell-specific molecule 1 | ESM1 | Q9NQ30 | −2.63 | −3.38 | −1.88 | 9.21E−12 | 4.29E−10 |
| Anthrax toxin receptor 2 | ANTXR2 | P58335 | −2.64 | −3.31 | −1.96 | 2.40E−14 | 1.96E−12 |
| ADP-ribosylation factor-like protein 11 | ARL11 | Q969Q4 | −2.65 | −3.56 | −1.74 | 1.30E−08 | 2.99E−07 |
| Hyaluronidase-1 | HYAL1 | Q12794 | −2.68 | −3.82 | −1.55 | 3.60E−06 | 4.92E−05 |
| Leucine-rich repeat transmembrane neuronal | LRRTM2 | O43300 | −2.69 | −3.49 | −1.89 | 5.83E−11 | 2.18E−09 |
| protein 2 | | | | | | | |
| Inter-alpha-trypsin inhibitor heavy chain H1 | ITIH1 | P19827 | −2.7 | −4 | −1.41 | 4.52E−05 | 4.76E−04 |
| Alpha-2-macroglobulin | A2M | P01023 | −2.71 | −3.5 | −1.93 | 1.51E−11 | 6.54E−10 |
| Netrin receptor UNC5D | UNC5D | Q6UXZ4 | −2.73 | −3.41 | −2.06 | 2.45E−15 | 2.39E−13 |
| ADAMTS-like protein 1 | ADAMTSL1 | Q8N6G6 | −2.74 | −3.55 | −1.92 | 6.30E−11 | 2.34E−09 |
| Neuroplastin | NPTN | Q9Y639 | −2.76 | −3.57 | −1.95 | 2.88E−11 | 1.16E−09 |
| Cystatin-M | CST6 | Q15828 | −2.81 | −3.56 | −2.07 | 1.39E−13 | 1.02E−11 |
| Cell adhesion molecule 2 | CADM2 | Q8N3J6 | −2.84 | −3.66 | −2.02 | 1.38E−11 | 6.03E−10 |
| Alpha-2-antiplasmin | SERPINF2 | P08697 | −2.86 | −4.69 | −1.03 | 2.22E−03 | 1.40E−02 |
| Potassium voltage-gated channel subfamily E | KCNE5 | Q9UJ90 | −2.87 | −3.77 | −1.97 | 4.05E−10 | 1.23E−08 |
| regulatory beta subunit 5: Cytoplasmic domain | | | | | | | |
| Protein SET | SET | Q01105 | −2.88 | −4.65 | −1.12 | 1.37E−03 | 9.27E−03 |
| S-methyl-5′-thioadenosine phosphorylase | MTAP | Q13126 | −2.89 | −4.17 | −1.6 | 1.10E−05 | 1.36E−04 |
| Vesicular, overexpressed in cancer, prosurvival | VOPP1 | Q96AW1 | −2.91 | −3.6 | −2.23 | 9.36E−17 | 1.37E−14 |
| protein 1 | | | | | | | |
| C1GALT1-specific chaperone 1 | C1GALT1C1 | Q96EU7 | −2.91 | −4.24 | −1.58 | 1.79E−05 | 2.10E−04 |
| Prostaglandin F2 receptor negative regulator | PTGFRN | Q9P2B2 | −2.92 | −3.64 | −2.21 | 1.78E−15 | 1.85E−13 |
| Syntaxin-1A | STX1A | Q16623 | −2.93 | −3.69 | −2.18 | 3.92E−14 | 3.10E−12 |
| Neuronal growth regulator 1 | NEGR1 | Q7Z3B1 | −2.96 | −4.18 | −1.74 | 2.08E−06 | 3.03E−05 |
| Neural cell adhesion molecule 2 | NCAM2 | O15394 | −2.98 | −3.69 | −2.27 | 2.35E−16 | 3.08E−14 |
| Sodium/potassium-transporting ATPase subunit | ATP1B2 | P14415 | −3.02 | −3.84 | −2.2 | 5.52E−13 | 3.30E−11 |
| beta-2 | | | | | | | |
| Heat shock 70 kDa protein 1A | HSPA1A | P0DMV8 | −3.04 | −4.13 | −1.95 | 4.68E−08 | 9.76E−07 |
| Neurocan core protein | NCAN | O14594 | −3.04 | −3.6 | −2.47 | 2.53E−25 | 4.20E−22 |
| Leukemia inhibitory factor receptor | LIFR | P42702 | −3.04 | −4.07 | −2 | 8.94E−09 | 2.15E−07 |
| Receptor-type tyrosine-protein phosphatase eta | PTPRJ | Q12913 | −3.04 | −4.11 | −1.98 | 2.42E−08 | 5.26E−07 |
| Seizure 6-like protein | SEZ6L | Q9BYH1 | −3.05 | −3.99 | −2.11 | 2.48E−10 | 7.92E−09 |
| Carbonic anhydrase 4 | CA4 | P22748 | −3.06 | −4.09 | −2.03 | 6.74E−09 | 1.64E−07 |
| Ciliary neurotrophic factor receptor subunit | CNTFR | P26992 | −3.07 | −4.04 | −2.1 | 6.51E−10 | 1.93E−08 |
| alpha | | | | | | | |
| Leucine-rich repeats and immunoglobulin-like | LRIG3 | Q6UXM1 | −3.09 | −4.15 | −2.04 | 1.03E−08 | 2.43E−07 |
| domains protein 3 | | | | | | | |
| Ephrin type-A receptor 4 | EPHA4 | P54764 | −3.11 | −4.25 | −1.97 | 9.02E−08 | 1.78E−06 |
| Gelsolin | GSN | P06396 | −3.12 | −4.6 | −1.64 | 3.64E−05 | 3.95E−04 |
| Kallistatin | SERPINA4 | P29622 | −3.12 | −4.51 | −1.73 | 1.10E−05 | 1.36E−04 |
| Prothrombin | F2 | P00734 | −3.13 | −4.94 | −1.31 | 7.32E−04 | 5.49E−03 |
| Coiled-coil domain-containing protein 126 | CCDC126 | Q96EE4 | −3.19 | −3.89 | −2.49 | 8.71E−19 | 2.17E−16 |
| Voltage-dependent calcium channel subunit | CACNA2D3 | Q8IZS8 | −3.23 | −3.93 | −2.53 | 2.63E−19 | 8.19E−17 |
| alpha-2/delta-3 | | | | | | | |
| IgLON family member 5 | IGLON5 | A6NGN9 | −3.25 | −3.99 | −2.51 | 8.42E−18 | 1.68E−15 |
| Secretogranin-3 | SCG3 | Q8WXD2 | −3.25 | −4.01 | −2.5 | 4.04E−17 | 6.87E−15 |
| Complement component 1 Q subcomponent- | C1QBP | Q07021 | −3.29 | −4.98 | −1.6 | 1.33E−04 | 1.23E−03 |
| binding protein, mitochondrial | | | | | | | |
| Monocyte differentiation antigen CD14, | CD14 | P08571 | −3.31 | −4.38 | −2.24 | 1.58E−09 | 4.45E−08 |
| soluble | | | | | | | |
| Anthrax toxin receptor 1 | ANTXR1 | Q9H6X2 | −3.33 | −4.01 | −2.65 | 1.29E−21 | 7.13E−19 |
| Apolipoprotein M | APOM | O95445 | −3.5 | −4.45 | −2.55 | 5.32E−13 | 3.23E−11 |
| Protein SCO1 homolog, mitochondrial | SCO1 | O75880 | −3.53 | −4.63 | −2.43 | 3.21E−10 | 1.01E−08 |
| Vitamin K-dependent protein C | PROC | P04070 | −3.57 | −4.82 | −2.32 | 2.45E−08 | 5.31E−07 |
| Cytoskeleton-associated protein 2 | CKAP2 | Q8WWK9 | −3.58 | −5.08 | −2.07 | 3.28E−06 | 4.52E−05 |
| Collagen alpha-1(XIII) chain | COL13A1 | Q5TAT6 | −3.64 | −4.71 | −2.58 | 2.17E−11 | 9.08E−10 |
| Hemopexin | HPX | P02790 | −3.65 | −5.38 | −1.92 | 3.51E−05 | 3.85E−04 |
| Platelet endothelial aggregation receptor | PEAR1 | Q5VY43 | −3.7 | −4.97 | −2.43 | 1.33E−08 | 3.04E−07 |
| 1: Extracellular domain | | | | | | | |
| Receptor-type tyrosine-protein phosphatase | PTPRD | P23468 | −3.71 | −4.45 | −2.96 | 2.18E−22 | 1.81E−19 |
| delta | | | | | | | |
| Transmembrane protein 132B | TMEM132B | Q14DG7 | −3.72 | −4.62 | −2.81 | 1.25E−15 | 1.35E−13 |
| Mannosyl-oligosaccharide 1,2-alpha- | MAN1A2 | O60476 | −3.73 | −5.18 | −2.28 | 4.89E−07 | 8.17E−06 |
| mannosidase IB | | | | | | | |
| Contactin-1 | CNTN1 | Q12860 | −3.75 | −4.76 | −2.74 | 4.99E−13 | 3.07E−11 |
| C-type lectin domain family 4 member M | CLEC4M | Q9H2X3 | −3.89 | −5.97 | −1.82 | 2.37E−04 | 2.02E−03 |
| N-acetylglucosamine-1-phosphodiester alpha- | NAGPA | Q9UK23 | −3.93 | −4.86 | −2.99 | 3.60E−16 | 4.38E−14 |
| N-acetylglucosaminidase | | | | | | | |
| Contactin-4 | CNTN4 | Q8TWV2 | −4.15 | −5.17 | −3.14 | 1.74E−15 | 1.85E−13 |
| NT-3 growth factor receptor | NTRK3 | Q16288 | −4.16 | −5.02 | −3.31 | 1.89E−21 | 8.99E−19 |
| Neural cell adhesion molecule L1-like protein | CHL1 | O00533 | −4.16 | −5.51 | −2.81 | 1.80E−09 | 5.00E−08 |
| Alpha-2-HS-glycoprotein | AHSG | P02765 | −4.22 | −5.82 | −2.63 | 2.27E−07 | 4.09E−06 |
| B melanoma antigen 3 | BAGE3 | Q86Y29 | −4.24 | −5.4 | −3.09 | 8.99E−13 | 4.82E−11 |
| Vitamin D-binding protein | GC | P02774 | −4.28 | −5.58 | −2.98 | 1.30E−10 | 4.52E−09 |
| Neural cell adhesion molecule 1, 120 kDa | NCAM1 | P13591 | −4.42 | −5.33 | −3.51 | 3.69E−21 | 1.53E−18 |
| isoform | | | | | | | |
| Matrix-remodeling-associated protein 8: | MXRA8 | Q9BRK3 | −4.44 | −5.26 | −3.61 | 1.50E−25 | 3.73E−22 |
| Extracellular domain | | | | | | | |
| Glycosaminoglycan xylosylkinase | FAM20B | O75063 | −4.51 | −5.74 | −3.29 | 5.86E−13 | 3.43E−11 |
| Beta-1,4-galactosyltransferase 2 | B4GALT2 | O60909 | −4.53 | −5.68 | −3.37 | 2.12E−14 | 1.76E−12 |
| Serotransferrin | TF | P02787 | −4.61 | −6.43 | −2.79 | 7.18E−07 | 1.15E−05 |
| Zinc-alpha-2-glycoprotein | AZGP1 | P25311 | −4.67 | −5.96 | −3.39 | 1.25E−12 | 6.55E−11 |
| Heparan-sulfate 6-O-sulfotransferase 3 | HS6ST3 | Q8IZP7 | −5.06 | −6.11 | −4 | 1.08E−20 | 3.57E−18 |
| Neuropeptide S | NPS | P0C0P6 | −5.12 | −6.43 | −3.8 | 3.43E−14 | 2.75E−12 |
| Inter-alpha-trypsin inhibitor heavy chain H2 | ITIH2 | P19823 | −5.34 | −6.8 | −3.88 | 1.01E−12 | 5.35E−11 |
| Epidermal growth factor receptor | EGFR | P00533 | −5.91 | −7.31 | −4.51 | 1.63E−16 | 2.19E−14 |
| Neurogenic locus notch homolog protein 1 | NOTCH1 | P46531 | −5.92 | −7.6 | −4.24 | 5.92E−12 | 2.78E−10 |
| Serum albumin | ALB | P02768 | −6.19 | −7.68 | −4.71 | 4.53E−16 | 5.37E−14 |
| Apolipoprotein C-I | APOC1 | P02654 | −6.31 | −8.01 | −4.62 | 3.98E−13 | 2.54E−11 |
| Antithrombin-III | SERPINC1 | P01008 | −6.72 | −8.65 | −4.79 | 1.03E−11 | 4.75E−10 |

In multivariable regression in COPDGene, 852 proteins (818 GeneIDs) were significant for QIA, with 561 proteins positively and 291 negatively associated with QIA (Table 4).

TABLE 4
Multivariable regression analysis of QIA in COPDGene

| | | | Percent | | | | |
|---|---|---|---|---|---|---|---|
| | Entrez gene | UniProt | QIA per | Upper | Lower | p- | FDR p- |
| Protein name | name | ID | ln(RFU) | 95% CI | 95% CI | value | value |
| Complement factor H | CFH | P08603 | 4.9 | 3.32 | 6.49 | 1.44E−09 | 7.49E−08 |
| Complement factor B | CFB | P00751 | 3.8 | 2.82 | 4.78 | 3.19E−14 | 4.67E−12 |
| Neuropilin-2 | NRP2 | O60462 | 3.37 | 2.43 | 4.31 | 2.03E−12 | 2.02E−10 |
| Roundabout homolog 2 | ROBO2 | Q9HCK4 | 2.98 | 2.11 | 3.85 | 1.87E−11 | 1.53E−09 |
| Lumican | LUM | P51884 | 2.87 | 2.21 | 3.54 | 2.22E−17 | 7.36E−15 |
| Serine protease HTRA1 | HTRA1 | Q92743 | 2.82 | 2.23 | 3.41 | 9.09E−21 | 4.53E−18 |
| Spondin-1 | SPON1 | Q9HCB6 | 2.76 | 2.1 | 3.41 | 2.17E−16 | 4.90E−14 |
| WAP four-disulfide core | WFDC2 | Q14508 | 2.74 | 2.35 | 3.13 | 3.07E−42 | 1.53E−38 |
| domain protein 2 | | | | | | | |
| C-X-C motif chemokine | CXCL16 | Q9H2A7 | 2.62 | 1.95 | 3.28 | 1.60E−14 | 2.57E−12 |
| 16 | | | | | | | |
| Neurofascin | NFASC | O94856 | 2.53 | 1.91 | 3.15 | 1.73E−15 | 3.08E−13 |
| EGF-containing fibulin- | EFEMP1 | Q12805 | 2.38 | 1.82 | 2.94 | 1.11E−16 | 2.75E−14 |
| like extracellular matrix | | | | | | | |
| protein 1 | | | | | | | |
| Complement factor I | CFI | P05156 | 2.3 | 1.05 | 3.54 | 3.07E−04 | 3.83E−03 |
| Leukocyte | LILRA5 | A6NI73 | 2.28 | 1.72 | 2.83 | 7.47E−16 | 1.49E−13 |
| immunoglobulin-like | | | | | | | |
| receptor subfamily A | | | | | | | |
| member 5 | | | | | | | |
| Immunoglobulin G | IGHG1 IGHG2 | P01857 | 2.27 | 1.37 | 3.18 | 9.23E−07 | 2.44E−05 |
| | IGHG3 IGHG4 | | | | | | |
| | IGK@ IGL@ | | | | | | |
| N-acetylglucosamine-1- | GNPTG | Q9UJJ9 | 2.21 | 1.36 | 3.05 | 3.15E−07 | 9.34E−06 |
| phosphotransferase subunit | | | | | | | |
| gamma | | | | | | | |
| Serine/arginine-rich | SRSF6 | Q13247 | 2.14 | 1.64 | 2.65 | 1.07E−16 | 2.75E−14 |
| splicing factor 6 | | | | | | | |
| Collagen alpha-1(XXVIII) | COL28A1 | Q2UY09 | 2.13 | 1.63 | 2.63 | 9.75E−17 | 2.70E−14 |
| chain | | | | | | | |
| C-C motif chemokine 18 | CCL18 | P55774 | 2.06 | 1.64 | 2.48 | 1.47E−21 | 9.18E−19 |
| Pigment epithelium- | SERPINF1 | P36955 | 2.06 | 0.98 | 3.15 | 2.05E−04 | 2.69E−03 |
| derived factor | | | | | | | |
| Collagen alpha-3(VI) | COL6A3 | P12111 | 2.05 | 1.54 | 2.57 | 8.47E−15 | 1.41E−12 |
| chain: Bovine pancreatic | | | | | | | |
| trypsin inhibitor/Kunitz | | | | | | | |
| inhibitor domain, isoform | | | | | | | |
| 1 | | | | | | | |
| Complement C1q tumor | C1QTNF1 | Q9BXJ1 | 2.03 | 1.27 | 2.79 | 1.69E−07 | 5.35E−06 |
| necrosis factor-related | | | | | | | |
| protein 1 | | | | | | | |
| Lipopolysaccharide- | LBP | P18428 | 2.01 | 1.17 | 2.84 | 2.45E−06 | 5.98E−05 |
| binding protein | | | | | | | |
| Fibulin-1 | FBLN1 | P23142 | 2.01 | 1.16 | 2.87 | 4.05E−06 | 9.37E−05 |
| Beta-2-microglobulin | B2M | P61769 | 1.97 | 1.52 | 2.43 | 3.49E−17 | 1.09E−14 |
| ADAMTS-like protein 2 | ADAMTSL2 | Q86TH1 | 1.96 | 1.52 | 2.4 | 2.42E−18 | 9.26E−16 |
| Olfactomedin-like protein | OLFML3 | Q9NRN5 | 1.94 | 1.25 | 2.64 | 4.20E−08 | 1.60E−06 |
| 3 | | | | | | | |
| R-spondin-4 | RSPO4 | Q2I0M5 | 1.92 | 1.35 | 2.49 | 4.01E−11 | 2.85E−09 |
| Complement component | C9 | P02748 | 1.91 | 1.29 | 2.52 | 1.24E−09 | 6.65E−08 |
| C9 | | | | | | | |
| Insulin-like growth factor- | IGFBP7 | Q16270 | 1.91 | 1.18 | 2.65 | 3.60E−07 | 1.05E−05 |
| binding protein 7 | | | | | | | |
| Pulmonary surfactant- | SFTPB | P07988 | 1.9 | 1.6 | 2.2 | 6.26E−35 | 1.54E−31 |
| associated protein B | | | | | | | |
| Alpha-1-microglobulin | AMBP | P02760 | 1.87 | 1.05 | 2.69 | 8.84E−06 | 1.80E−04 |
| Tumor necrosis factor | TNFSF15 | O95150 | 1.85 | 1.28 | 2.41 | 1.47E−10 | 9.62E−09 |
| ligand superfamily | | | | | | | |
| member 15 | | | | | | | |
| Tryptophan--tRNA ligase, | WARS | P23381 | 1.81 | 1.3 | 2.32 | 3.15E−12 | 2.96E−10 |
| cytoplasmic | | | | | | | |
| CUB domain-containing | CDCP1 | Q9H5V8 | 1.78 | 1.5 | 2.06 | 9.26E−35 | 1.54E−31 |
| protein 1 | | | | | | | |
| Cystatin-C | CST3 | P01034 | 1.78 | 1.24 | 2.33 | 1.95E−10 | 1.23E−08 |
| Inactive tyrosine-protein | PTK7 | Q13308 | 1.78 | 1.08 | 2.48 | 6.34E−07 | 1.78E−05 |
| kinase 7 | | | | | | | |
| Plastin-2 | LCP1 | P13796 | 1.77 | 1.01 | 2.53 | 5.36E−06 | 1.18E−04 |
| Triggering receptor | TREM1 | Q9NP99 | 1.76 | 1.33 | 2.19 | 1.00E−15 | 1.85E−13 |
| expressed on myeloid cells | | | | | | | |
| 1 | | | | | | | |
| Tenascin | TNC | P24821 | 1.72 | 1.22 | 2.22 | 2.07E−11 | 1.66E−09 |
| Gamma-glutamyl | GGH | Q92820 | 1.72 | 1.19 | 2.26 | 2.81E−10 | 1.69E−08 |
| hydrolase | | | | | | | |
| Peptidyl-prolyl cis-trans | PPIC | P45877 | 1.71 | 1.14 | 2.27 | 2.87E−09 | 1.37E−07 |
| isomerase C | | | | | | | |
| Follistatin-related protein 1 | FSTL1 | Q12841 | 1.7 | 0.71 | 2.68 | 7.06E−04 | 7.22E−03 |
| C-C motif chemokine 21 | CCL21 | O00585 | 1.68 | 1.25 | 2.1 | 1.98E−14 | 3.08E−12 |
| Carbohydrate | CHST15 | Q7LFX5 | 1.68 | 0.89 | 2.47 | 3.08E−05 | 5.23E−04 |
| sulfotransferase 15 | | | | | | | |
| Tenascin | TNC | P24821 | 1.67 | 1.21 | 2.13 | 1.24E−12 | 1.32E−10 |
| Brain-specific serine | PRSS22 | Q9GZN4 | 1.64 | 1.11 | 2.17 | 1.50E−09 | 7.68E−08 |
| protease 4 | | | | | | | |
| Heat shock 70 kDa protein | HSPA1A | PODMV8 | 1.63 | 0.97 | 2.29 | 1.49E−06 | 3.81E−05 |
| 1A | | | | | | | |
| Microfibrillar-associated | MFAP2 | P55001 | 1.62 | 0.98 | 2.26 | 7.38E−07 | 2.03E−05 |
| protein 2 | | | | | | | |
| Sushi, von Willebrand | SVEP1 | Q4LDE5 | 1.62 | 1.24 | 2 | 6.75E−17 | 1.98E−14 |
| factor type A, EGF and | | | | | | | |
| pentraxin domain- | | | | | | | |
| containing protein 1: EGF- | | | | | | | |
| like domains 4-6 | | | | | | | |
| Macrophage mannose | MRC1 | P22897 | 1.62 | 1.03 | 2.21 | 6.87E−08 | 2.41E−06 |
| receptor 1 | | | | | | | |
| Neuropilin-1 | NRP | O14786 | 1.62 | 0.99 | 2.24 | 5.04E−07 | 1.44E−05 |
| Vesicular integral- | LMAN2 | Q12907 | 1.62 | 1.03 | 2.2 | 5.17E−08 | 1.89E−06 |
| membrane protein VIP36 | | | | | | | |
| Growth/differentiation | GDF15 | Q99988 | 1.61 | 1.31 | 1.91 | 1.24E−25 | 1.54E−22 |
| factor 15 | | | | | | | |
| Induced myeloid leukemia | MCL1 | Q07820 | 1.59 | 1.16 | 2.02 | 3.91E−13 | 4.75E−11 |
| cell differentiation protein | | | | | | | |
| Mcl-1 | | | | | | | |
| Vascular endothelial | VEGFA | P15692 | 1.59 | 0.87 | 2.32 | 1.75E−05 | 3.21E−04 |
| growth factor A | | | | | | | |
| Retinoic acid receptor | RARRES2 | Q99969 | 1.59 | 1.03 | 2.15 | 2.67E−08 | 1.04E−06 |
| responder protein 2 | | | | | | | |
| SPARC-related modular | SMOC2 | Q9H3U7 | 1.56 | 1.02 | 2.1 | 1.55E−08 | 6.30E−07 |
| calcium-binding protein 2 | | | | | | | |
| Twisted gastrulation | TWSG1 | Q9GZX9 | 1.56 | 0.94 | 2.19 | 9.08E−07 | 2.43E−05 |
| protein homolog 1 | | | | | | | |
| Axin-2 | AXIN2 | Q9Y2T1 | 1.52 | 1.02 | 2.03 | 3.47E−09 | 1.64E−07 |
| Kremen protein 1 | KREMEN1 | Q96MU8 | 1.51 | 0.64 | 2.37 | 6.47E−04 | 6.81E−03 |
| Sushi, von Willebrand | SVEP1 | Q4LDE5 | 1.5 | 1.11 | 1.89 | 7.35E−14 | 9.39E−12 |
| factor type A, EGF and | | | | | | | |
| pentraxin domain- | | | | | | | |
| containing protein 1: Sushi | | | | | | | |
| 15-18 | | | | | | | |
| Limbic system-associated | LSAMP | Q13449 | 1.5 | 0.67 | 2.33 | 3.82E−04 | 4.55E−03 |
| membrane protein | | | | | | | |
| Marginal zone B- and B1- | PACAP | Q8WU39 | 1.49 | 1.18 | 1.8 | 5.96E−21 | 3.30E−18 |
| cell-specific protein | | | | | | | |
| Tenascin | TNC | P24821 | 1.49 | 1.01 | 1.96 | 9.96E−10 | 5.45E−08 |
| Plexin-D1 | PLXND1 | Q9Y4D7 | 1.47 | 0.89 | 2.05 | 6.80E−07 | 1.90E−05 |
| Complement C2 | C2 | P06681 | 1.46 | 0.73 | 2.18 | 8.58E−05 | 1.26E−03 |
| Hepatitis A virus cellular | HAVCR2 | Q8TDQ0 | 1.46 | 0.97 | 1.95 | 4.19E−09 | 1.95E−07 |
| receptor 2 | | | | | | | |
| Plexin-B2 | PLXNB2 | O15031 | 1.46 | 0.73 | 2.19 | 8.22E−05 | 1.21E−03 |
| Ganglioside GM2 activator | GM2A | P17900 | 1.45 | 0.97 | 1.94 | 4.77E−09 | 2.16E−07 |
| Complement component | CD93 | Q9NPY3 | 1.43 | 0.79 | 2.06 | 1.09E−05 | 2.17E−04 |
| C1q receptor | | | | | | | |
| Complement component | C7 | P10643 | 1.43 | 0.59 | 2.26 | 8.11E−04 | 8.09E−03 |
| C7 | | | | | | | |
| Follistatin-related protein 3 | FSTL3 | O95633 | 1.43 | 0.89 | 1.98 | 2.86E−07 | 8.73E−06 |
| Transferrin receptor | TFRC | P02786 | 1.43 | 0.97 | 1.89 | 1.16E−09 | 6.27E−08 |
| protein 1: Extracellular | | | | | | | |
| domain | | | | | | | |
| Antileukoproteinase | SLPI | P03973 | 1.41 | 0.74 | 2.08 | 3.57E−05 | 5.91E−04 |
| Serine/arginine-rich | SRSF7 | Q16629 | 1.4 | 0.85 | 1.95 | 7.10E−07 | 1.98E−05 |
| splicing factor 7 | | | | | | | |
| E-selectin | SELE | P16581 | 1.4 | 1.05 | 1.74 | 4.60E−15 | 7.89E−13 |
| Multiple inositol | MINPP1 | Q9UNW1 | 1.4 | 0.57 | 2.22 | 8.91E−04 | 8.71E−03 |
| polyphosphate phosphatase | | | | | | | |
| 1 | | | | | | | |
| Ephrin type-B receptor 2 | EPHB2 | P29323 | 1.39 | 0.78 | 2 | 7.92E−06 | 1.64E−04 |
| Allograft inflammatory | AIF1L | Q9BQI0 | 1.38 | 0.82 | 1.95 | 1.59E−06 | 4.03E−05 |
| factor 1-like | | | | | | | |
| Granulins | GRN | P28799 | 1.38 | 0.69 | 2.08 | 1.04E−04 | 1.49E−03 |
| R-spondin-3 | RSPO3 | Q9BXY4 | 1.38 | 0.77 | 1.98 | 8.14E−06 | 1.66E−04 |
| Transaldolase | TALDO1 | P37837 | 1.37 | 0.86 | 1.88 | 1.61E−07 | 5.17E−06 |
| Butyrophilin subfamily 3 | BTN3A3 | O00478 | 1.36 | 0.97 | 1.74 | 8.83E−12 | 7.58E−10 |
| member A3 | | | | | | | |
| Fibrinogen | FGA FGB | P02679\|P02675\|P02671 | 1.36 | 0.54 | 2.18 | 1.10E−03 | 1.04E−02 |
| | FGG | | | | | | |
| Intercellular adhesion | ICAM5 | Q9UMF0 | 1.36 | 0.91 | 1.8 | 2.34E−09 | 1.14E−07 |
| molecule 5 | | | | | | | |
| C-X-C motif chemokine | CXCL13 | O43927 | 1.35 | 1.03 | 1.68 | 4.67E−16 | 9.68E−14 |
| 13 | | | | | | | |
| Chordin-like protein 2 | CHRDL2 | Q6WN34 | 1.35 | 0.92 | 1.78 | 8.02E−10 | 4.54E−08 |
| Mucin-16 | MUC16 | Q8WXI7 | 1.34 | 0.96 | 1.72 | 4.98E−12 | 4.43E−10 |
| Thrombospondin-2 | THBS2 | P35442 | 1.34 | 1.08 | 1.61 | 9.98E−23 | 8.28E−20 |
| Tumor necrosis factor | TNFRSF1B | P20333 | 1.34 | 0.95 | 1.73 | 2.53E−11 | 1.94E−09 |
| receptor superfamily | | | | | | | |
| member 1B | | | | | | | |
| Small ubiquitin-related | SUMO2 | P61956 | 1.32 | 0.95 | 1.7 | 4.40E−12 | 3.98E−10 |
| modifier 2 | | | | | | | |
| Alpha-1-antitrypsin | SERPINA1 | P01009 | 1.32 | 0.82 | 1.82 | 2.95E−07 | 8.91E−06 |
| RELT-like protein 1 | RELL1 | Q8IUW5 | 1.31 | 0.83 | 1.79 | 7.28E−08 | 2.52E−06 |
| Angiogenin | ANG | P03950 | 1.3 | 0.78 | 1.81 | 7.47E−07 | 2.04E−05 |
| Stanniocalcin-1 | STC1 | P52823 | 1.3 | 0.78 | 1.82 | 8.74E−07 | 2.37E−05 |
| Intercellular adhesion | ICAM5 | Q9UMF0 | 1.3 | 0.9 | 1.7 | 2.09E−10 | 1.30E−08 |
| molecule 5 | | | | | | | |
| Transferrin receptor | TFRC | P02786 | 1.3 | 0.92 | 1.67 | 8.38E−12 | 7.32E−10 |
| protein 1: Cytoplasmic | | | | | | | |
| domain | | | | | | | |
| Bone morphogenetic | BMP6 | P22004 | 1.3 | 0.73 | 1.86 | 8.01E−06 | 1.65E−04 |
| protein 6 | | | | | | | |
| Serine/threonine-protein | SMEK1 | Q6IN85 | 1.29 | 0.91 | 1.67 | 3.56E−11 | 2.57E−09 |
| phosphatase 4 regulatory | | | | | | | |
| subunit 3A | | | | | | | |
| Tumor necrosis factor | TNFRSF17 | Q02223 | 1.29 | 0.83 | 1.75 | 4.51E−08 | 1.69E−06 |
| receptor superfamily | | | | | | | |
| member 17 | | | | | | | |
| Macrophage-capping | CAPG | P40121 | 1.29 | 0.91 | 1.66 | 2.30E−11 | 1.82E−09 |
| protein | | | | | | | |
| Ribonuclease 4 | RNASE4 | P34096 | 1.29 | 0.56 | 2.03 | 5.59E−04 | 6.10E−03 |
| Neuroblastoma suppressor | NBL1 | P41271 | 1.27 | 0.9 | 1.65 | 2.40E−11 | 1.87E−09 |
| of tumorigenicity 1 | | | | | | | |
| WAP four-disulfide core | WFDC1 | Q9HC57 | 1.27 | 0.83 | 1.71 | 1.78E−08 | 7.15E−07 |
| domain protein 1 | | | | | | | |
| Retinoblastoma-like | RBL2 | Q08999 | 1.26 | 0.83 | 1.69 | 1.26E−08 | 5.24E−07 |
| protein 2 | | | | | | | |
| Core-binding factor | CBFB | Q13951 | 1.25 | 0.43 | 2.08 | 2.96E−03 | 2.22E−02 |
| subunit beta | | | | | | | |
| Golgi membrane protein 1 | GOLM1 | Q8NBJ4 | 1.25 | 0.9 | 1.59 | 1.33E−12 | 1.38E−10 |
| Coiled-coil domain- | CCDC80 | Q76M96 | 1.25 | 0.61 | 1.9 | 1.44E−04 | 1.98E−03 |
| containing protein 80 | | | | | | | |
| 14-3-3 protein sigma | SFN | P31947 | 1.25 | 0.81 | 1.69 | 3.19E−08 | 1.23E−06 |
| Thioredoxin domain- | TXNDC5 | Q8NBS9 | 1.24 | 0.82 | 1.65 | 4.01E−09 | 1.88E−07 |
| containing protein 5 | | | | | | | |
| HLA class I | HLA-G | P17693 | 1.24 | 0.36 | 2.11 | 5.64E−03 | 3.68E−02 |
| histocompatibility antigen, | | | | | | | |
| alpha chain G | | | | | | | |
| Semaphorin-6B | SEMA6B | Q9H3T3 | 1.24 | 0.67 | 1.8 | 1.67E−05 | 3.10E−04 |
| CD166 antigen | ALCAM | Q13740 | 1.24 | 0.41 | 2.08 | 3.52E−03 | 2.55E−02 |
| Membrane cofactor protein | CD46 | P15529 | 1.23 | 0.51 | 1.95 | 8.50E−04 | 8.36E−03 |
| Angiopoietin-2 | ANGPT2 | O15123 | 1.23 | 0.88 | 1.57 | 2.67E−12 | 2.56E−10 |
| C-C motif chemokine 3 | CCL3 | P10147 | 1.23 | 0.83 | 1.63 | 1.99E−09 | 9.93E−08 |
| Trefoil factor 3 | TFF3 | Q07654 | 1.22 | 0.82 | 1.62 | 2.77E−09 | 1.34E−07 |
| Interleukin-9 | IL9 | P15248 | 1.22 | 0.67 | 1.77 | 1.40E−05 | 2.69E−04 |
| Calsyntenin-3 | CLSTN3 | Q9BQT9 | 1.22 | 0.82 | 1.61 | 1.83E−09 | 9.19E−08 |
| Tyrosine-protein kinase | ROR2 | Q01974 | 1.22 | 0.82 | 1.62 | 2.08E−09 | 1.03E−07 |
| transmembrane receptor | | | | | | | |
| ROR2 | | | | | | | |
| Beta-1,4- | B4GALT1 | P15291 | 1.21 | 0.42 | 2 | 2.83E−03 | 2.14E−02 |
| galactosyltransferase 1 | | | | | | | |
| Myosin light chain 6B | MYL6B | P14649 | 1.21 | 0.77 | 1.66 | 8.88E−08 | 3.05E−06 |
| Pulmonary surfactant- | SFTPD | P35247 | 1.21 | 0.97 | 1.45 | 6.80E−23 | 6.77E−20 |
| associated protein D | | | | | | | |
| Nectin-2 | PVRL2 | Q92692 | 1.21 | 0.5 | 1.92 | 8.60E−04 | 8.44E−03 |
| Matrix Gla protein | MGP | P08493 | 1.21 | 0.63 | 1.79 | 4.31E−05 | 6.95E−04 |
| Leucine-rich repeats and | LRIG1 | Q96JA1 | 1.2 | 0.61 | 1.78 | 5.97E−05 | 9.14E−04 |
| immunoglobulin-like | | | | | | | |
| domains protein 1 | | | | | | | |
| C-C motif chemokine 14 | CCL14 | Q16627 | 1.2 | 0.88 | 1.53 | 2.91E−13 | 3.62E−11 |
| Bone morphogenetic | BMP7 | P18075 | 1.2 | 0.66 | 1.74 | 1.50E−05 | 2.85E−04 |
| protein 7 | | | | | | | |
| Ephrin type-A receptor 2 | EPHA2 | P29317 | 1.2 | 0.75 | 1.65 | 2.30E−07 | 7.07E−06 |
| Ephrin-A4 | EFNA4 | P52798 | 1.19 | 0.62 | 1.76 | 4.65E−05 | 7.38E−04 |
| Tumor necrosis factor | TNFRSF1B | P20333 | 1.19 | 0.81 | 1.57 | 9.32E−10 | 5.21E−08 |
| receptor superfamily | | | | | | | |
| member 1B | | | | | | | |
| Low affinity | FCGR3B | O75015 | 1.19 | 0.79 | 1.6 | 7.36E−09 | 3.16E−07 |
| immunoglobulin gamma | | | | | | | |
| Fc region receptor III-B | | | | | | | |
| Prenylcysteine oxidase- | PCYOX1L | Q8NBM8 | 1.19 | 0.61 | 1.77 | 6.44E−05 | 9.75E−04 |
| like | | | | | | | |
| Poly(U)-binding-splicing | PUF60 | Q9UHX1 | 1.18 | 0.65 | 1.72 | 1.61E−05 | 3.01E−04 |
| factor PUF60 | | | | | | | |
| Heat shock 70 kDa protein | HSPA1A | PODMV8 | 1.18 | 0.64 | 1.72 | 1.89E−05 | 3.45E−04 |
| 1A | | | | | | | |
| Gamma-aminobutyric acid | GABARAP | O95166 | 1.18 | 0.68 | 1.68 | 4.03E−06 | 9.37E−05 |
| receptor-associated protein | | | | | | | |
| Calcium-binding and | CALCOCO2 | Q13137 | 1.16 | 0.42 | 1.91 | 2.21E−03 | 1.79E−02 |
| coiled-coil domain- | | | | | | | |
| containing protein 2 | | | | | | | |
| Protein S100-A11 | S100A11 | P31949 | 1.16 | 0.62 | 1.69 | 2.06E−05 | 3.71E−04 |
| Matrilysin | MMP7 | P09237 | 1.16 | 0.88 | 1.44 | 3.01E−16 | 6.52E−14 |
| Thrombospondin-4 | THBS4 | P35443 | 1.16 | 0.63 | 1.69 | 2.03E−05 | 3.68E−04 |
| Trefoil factor 3 | TFF3 | Q07654 | 1.16 | 0.71 | 1.61 | 4.01E−07 | 1.15E−05 |
| Nuclease-sensitive | YBX1 | P67809 | 1.16 | 0.79 | 1.52 | 5.40E−10 | 3.16E−08 |
| element-binding protein 1 | | | | | | | |
| Fibroblast growth factor- | FGFBP3 | Q8TAT2 | 1.15 | 0.59 | 1.72 | 6.54E−05 | 9.86E−04 |
| binding protein 3 | | | | | | | |
| Immunoglobulin | IGSF3 | O75054 | 1.15 | 0.7 | 1.61 | 7.18E−07 | 1.99E−05 |
| superfamily member 3 | | | | | | | |
| Keratocan | KERA | O60938 | 1.14 | 0.6 | 1.67 | 3.77E−05 | 6.22E−04 |
| T-cell immunoglobulin | TIMD4 | Q96H15 | 1.14 | 0.75 | 1.52 | 5.40E−09 | 2.38E−07 |
| and mucin domain- | | | | | | | |
| containing protein 4 | | | | | | | |
| Legumain | LGMN | Q99538 | 1.14 | 0.49 | 1.79 | 5.45E−04 | 6.00E−03 |
| IGF-like family receptor 1 | IGFLR1 | Q9H665 | 1.13 | 0.8 | 1.46 | 3.18E−11 | 2.37E−09 |
| Chloride intracellular | CLIC5 | Q9NZA1 | 1.12 | 0.68 | 1.57 | 8.93E−07 | 2.40E−05 |
| channel protein 5 | | | | | | | |
| Low-density lipoprotein | LRP11 | Q86VZ4 | 1.12 | 0.5 | 1.75 | 4.20E−04 | 4.89E−03 |
| receptor-related protein 11 | | | | | | | |
| C-C motif chemokine 15 | CCL15 | Q16663 | 1.12 | 0.71 | 1.52 | 6.37E−08 | 2.26E−06 |
| Ribonuclease K6 | RNASE6 | Q93091 | 1.12 | 0.75 | 1.49 | 4.26E−09 | 1.97E−07 |
| Vascular endothelial | FLT4 | P35916 | 1.11 | 0.59 | 1.64 | 3.40E−05 | 5.66E−04 |
| growth factor receptor 3 | | | | | | | |
| Junctional adhesion | JAM2 | P57087 | 1.11 | 0.37 | 1.85 | 3.30E−03 | 2.42E−02 |
| molecule B | | | | | | | |
| Complement C1r | C1RL | Q9NZP8 | 1.1 | 0.32 | 1.88 | 5.85E−03 | 3.78E−02 |
| subcomponent-like protein | | | | | | | |
| Interleukin enhancer- | ILF3 | Q12906 | 1.09 | 0.53 | 1.64 | 1.17E−04 | 1.65E−03 |
| binding factor 3 | | | | | | | |
| Protein S100-A16 | S100A16 | Q96FQ6 | 1.09 | 0.43 | 1.75 | 1.13E−03 | 1.06E−02 |
| C-type mannose receptor 2 | MRC2 | Q9UBG0 | 1.09 | 0.5 | 1.69 | 3.34E−04 | 4.10E−03 |
| Granzyme A | GZMA | P12544 | 1.09 | 0.65 | 1.53 | 1.25E−06 | 3.23E−05 |
| Peptidyl-glycine alpha- | PAM | P19021 | 1.09 | 0.42 | 1.76 | 1.33E−03 | 1.20E−02 |
| amidating monooxygenase | | | | | | | |
| Sulfatase-modifying factor | SUMF2 | Q8NBJ7 | 1.09 | 0.46 | 1.73 | 7.26E−04 | 7.41E−03 |
| 2 | | | | | | | |
| Leukocyte | LILRA5 | A6NI73 | 1.09 | 0.66 | 1.51 | 5.20E−07 | 1.48E−05 |
| immunoglobulin-like | | | | | | | |
| receptor subfamily A | | | | | | | |
| member 5 | | | | | | | |
| Immunoglobulin A | IGHA1 | P01877\|P01876 | 1.08 | 0.78 | 1.37 | 9.15E−13 | 1.01E−10 |
| | IGHA2 | | | | | | |
| C4b-binding protein alpha | C4BPA | P04003 | 1.08 | 0.39 | 1.77 | 2.09E−03 | 1.72E−02 |
| chain | | | | | | | |
| Gamma-aminobutyric acid | GABARAPL1 | Q9H0R8 | 1.07 | 0.58 | 1.56 | 2.06E−05 | 3.71E−04 |
| receptor-associated | | | | | | | |
| protein-like 1 | | | | | | | |
| Metalloproteinase inhibitor | TIMP1 | P01033 | 1.07 | 0.52 | 1.61 | 1.18E−04 | 1.66E−03 |
| 1 | | | | | | | |
| Transmembrane | GPNMB | Q14956 | 1.07 | 0.48 | 1.66 | 3.73E−04 | 4.47E−03 |
| glycoprotein | | | | | | | |
| NMB: Extracellular domain | | | | | | | |
| Endoplasmic reticulum | ERLEC1 | Q96DZ1 | 1.07 | 0.57 | 1.57 | 2.43E−05 | 4.27E−04 |
| lectin 1 | | | | | | | |
| Peroxidasin homolog | PXDN | Q92626 | 1.06 | 0.77 | 1.36 | 1.37E−12 | 1.39E−10 |
| Macrophage | MMP12 | P39900 | 1.06 | 0.76 | 1.35 | 2.45E−12 | 2.40E−10 |
| metalloelastase | | | | | | | |
| Macrophage scavenger | MSR1 | P21757 | 1.05 | 0.74 | 1.36 | 5.61E−11 | 3.94E−09 |
| receptor types I and | | | | | | | |
| II: Extracellular domain | | | | | | | |
| Folate receptor beta | FOLR2 | P14207 | 1.05 | 0.4 | 1.71 | 1.51E−03 | 1.32E−02 |
| BPI fold-containing family | BPIFB1 | Q8TDL5 | 1.04 | 0.76 | 1.32 | 6.78E−13 | 7.97E−11 |
| B member 1 | | | | | | | |
| BMP-binding endothelial | BMPER | Q8N8U9 | 1.04 | 0.47 | 1.6 | 3.42E−04 | 4.18E−03 |
| regulator protein | | | | | | | |
| Bone morphogenetic | BMP4 | P12644 | 1.04 | 0.58 | 1.51 | 9.25E−06 | 1.87E−04 |
| protein 4 | | | | | | | |
| NADH-cytochrome b5 | CYB5R1 | Q9UHQ9 | 1.04 | 0.37 | 1.71 | 2.35E−03 | 1.88E−02 |
| reductase 1 | | | | | | | |
| Collagen Type III | COL3A1 | P02461 | 1.04 | 0.46 | 1.62 | 4.24E−04 | 4.91E−03 |
| Heat shock 70 kDa protein | HSPA1B | P0DMV9 | 1.04 | 0.59 | 1.49 | 6.59E−06 | 1.40E−04 |
| 1B | | | | | | | |
| Elafin | PI3 | P19957 | 1.04 | 0.72 | 1.36 | 1.52E−10 | 9.71E−09 |
| Heat shock cognate 71 | HSPA8 | P11142 | 1.04 | 0.54 | 1.55 | 5.69E−05 | 8.78E−04 |
| kDa protein | | | | | | | |
| Interleukin-3 receptor | IL3RA | P26951 | 1.03 | 0.65 | 1.4 | 1.06E−07 | 3.59E−06 |
| subunit alpha | | | | | | | |
| Protein dpy-30 homolog | DPY30 | Q9C005 | 1.03 | 0.66 | 1.4 | 6.84E−08 | 2.41E−06 |
| Hepatoma-derived growth | HDGF | P51858 | 1.03 | 0.59 | 1.47 | 3.77E−06 | 8.85E−05 |
| factor | | | | | | | |
| PC4 and SFRS1- | PSIP1 | O75475 | 1.03 | 0.59 | 1.47 | 4.24E−06 | 9.69E−05 |
| interacting protein | | | | | | | |
| Erythropoietin | EPO | P01588 | 1.03 | 0.7 | 1.35 | 9.71E−10 | 5.37E−08 |
| Prostaglandin-H2 D- | PTGDS | P41222 | 1.02 | 0.55 | 1.48 | 1.98E−05 | 3.61E−04 |
| isomerase | | | | | | | |
| Fibulin-5 | FBLN5 | Q9UBX5 | 1.02 | 0.53 | 1.51 | 5.03E−05 | 7.87E−04 |
| Galectin-4 | LGALS4 | P56470 | 1.02 | 0.54 | 1.49 | 2.56E−05 | 4.47E−04 |
| Gremlin-2 | GREM2 | Q9H772 | 1.02 | 0.48 | 1.57 | 2.21E−04 | 2.88E−03 |
| Kallikrein-10 | KLK10 | O43240 | 1.02 | 0.64 | 1.41 | 1.60E−07 | 5.17E−06 |
| Polypeptide N- | GALNT1 | Q10472 | 1.02 | 0.31 | 1.73 | 4.88E−03 | 3.30E−02 |
| acetylgalactosaminyltransferase | | | | | | | |
| 1 | | | | | | | |
| Protocadherin alpha-C2 | PCDHAC2 | Q9Y5I4 | 1.02 | 0.45 | 1.6 | 4.82E−04 | 5.48E−03 |
| Insulin-like growth factor- | IGFBP4 | P22692 | 1.01 | 0.4 | 1.62 | 1.19E−03 | 1.10E−02 |
| binding protein 4 | | | | | | | |
| B-cell differentiation | CD72 | P21854 | 1.01 | 0.46 | 1.57 | 3.33E−04 | 4.10E−03 |
| antigen CD72 | | | | | | | |
| Intercellular adhesion | ICAM1 | P05362 | 1 | 0.66 | 1.34 | 7.64E−09 | 3.25E−07 |
| molecule 1 | | | | | | | |
| Stromal cell-derived factor | SDF2L1 | Q9HCN8 | 1 | 0.39 | 1.6 | 1.31E−03 | 1.19E−02 |
| 2-like protein 1 | | | | | | | |
| Spondin-2 | SPON2 | Q9BUD6 | 1 | 0.62 | 1.38 | 3.02E−07 | 9.06E−06 |
| Interleukin-15 receptor | IL15RA | Q13261 | 0.99 | 0.62 | 1.36 | 1.93E−07 | 5.99E−06 |
| subunit alpha | | | | | | | |
| Delta-like protein 1 | DLL1 | O00548 | 0.99 | 0.44 | 1.54 | 4.18E−04 | 4.88E−03 |
| Adenosine deaminase | CECR1 | Q9NZK5 | 0.99 | 0.69 | 1.3 | 1.52E−10 | 9.71E−09 |
| CECR1 | | | | | | | |
| Dolichyl- | RPN1 | P04843 | 0.98 | 0.59 | 1.37 | 9.24E−07 | 2.44E−05 |
| diphosphooligosaccharide-- | | | | | | | |
| protein | | | | | | | |
| glycosyltransferase subunit | | | | | | | |
| 1: Cytoplasmic domain | | | | | | | |
| Heat shock 70 kDa protein | HSPA1A | P0DMV8 | 0.98 | 0.56 | 1.4 | 5.64E−06 | 1.23E−04 |
| 1A | | | | | | | |
| Noelin | OLFM1 | Q99784 | 0.98 | 0.54 | 1.42 | 1.40E−05 | 2.69E−04 |
| Transmembrane emp24 | TMED10 | P49755 | 0.98 | 0.56 | 1.41 | 6.00E−06 | 1.30E−04 |
| domain-containing protein | | | | | | | |
| 10 | | | | | | | |
| Microfibrillar-associated | MFAP2 | P55001 | 0.98 | 0.36 | 1.6 | 1.96E−03 | 1.63E−02 |
| protein 2 | | | | | | | |
| Ran-binding protein 3 | RANBP3 | Q9H6Z4 | 0.97 | 0.49 | 1.44 | 6.11E−05 | 9.30E−04 |
| Selenoprotein M | SELM | Q8WWX9 | 0.97 | 0.52 | 1.43 | 3.06E−05 | 5.21E−04 |
| Endostatin | COL18A1 | P39060 | 0.97 | 0.44 | 1.5 | 3.26E−04 | 4.03E−03 |
| Chordin-like protein 1 | CHRDL1 | Q9BU40 | 0.97 | 0.42 | 1.53 | 5.72E−04 | 6.19E−03 |
| Protein CYR61 | CYR61 | O00622 | 0.97 | 0.53 | 1.41 | 1.65E−05 | 3.07E−04 |
| High mobility group | HMG20A | Q9NP66 | 0.96 | 0.34 | 1.58 | 2.54E−03 | 2.00E−02 |
| protein 20A | | | | | | | |
| Serpin B8 | SERPINB8 | P50452 | 0.96 | 0.41 | 1.51 | 5.95E−04 | 6.38E−03 |
| Phospholipase A2, | PLA2G2A | P14555 | 0.96 | 0.65 | 1.27 | 1.44E−09 | 7.49E−08 |
| membrane associated | | | | | | | |
| Fibrinogen gamma chain | FGG | P02679 | 0.96 | 0.45 | 1.47 | 2.23E−04 | 2.90E−03 |
| Tumor necrosis factor | TNFRSF19 | Q9NS68 | 0.96 | 0.52 | 1.41 | 2.44E−05 | 4.28E−04 |
| receptor superfamily | | | | | | | |
| member 19 | | | | | | | |
| Tenascin | TNC | P24821 | 0.96 | 0.62 | 1.3 | 4.04E−08 | 1.55E−06 |
| B melanoma antigen 2 | BAGE2 | Q86Y30 | 0.96 | 0.54 | 1.39 | 1.05E−05 | 2.09E−04 |
| NTF2-related export | NXT1 | Q9UKK6 | 0.96 | 0.5 | 1.43 | 4.92E−05 | 7.75E−04 |
| protein 1 | | | | | | | |
| C-C motif chemokine 22 | CCL22 | O00626 | 0.95 | 0.61 | 1.29 | 5.88E−08 | 2.11E−06 |
| Fibrinogen-like protein 1 | FGL1 | Q08830 | 0.95 | 0.66 | 1.24 | 1.08E−10 | 7.33E−09 |
| Phospholipase B-like 1 | PLBD1 | Q6P4A8 | 0.95 | 0.48 | 1.42 | 7.21E−05 | 1.07E−03 |
| Neural proliferation | NPDC1 | Q9NQX5 | 0.94 | 0.44 | 1.43 | 1.93E−04 | 2.55E−03 |
| differentiation and control | | | | | | | |
| protein 1 | | | | | | | |
| R-spondin-3 | RSPO3 | Q9BXY4 | 0.94 | 0.41 | 1.47 | 5.26E−04 | 5.87E−03 |
| Cadherin-11: Extracellular | CDH11 | P55287 | 0.94 | 0.72 | 1.15 | 5.24E−18 | 1.86E−15 |
| domain | | | | | | | |
| Acyl-CoA-binding | ACBD6 | Q9BR61 | 0.94 | 0.4 | 1.48 | 6.35E−04 | 6.71E−03 |
| domain-containing protein | | | | | | | |
| 6 | | | | | | | |
| Tumor necrosis factor | TNFRSF1A | P19438 | 0.94 | 0.54 | 1.33 | 3.85E−06 | 9.00E−05 |
| receptor superfamily | | | | | | | |
| member 1A | | | | | | | |
| Protein FAM189A2 | FAM189A2 | Q15884 | 0.94 | 0.39 | 1.49 | 7.80E−04 | 7.88E−03 |
| Thrombospondin-3 | THBS3 | P49746 | 0.94 | 0.49 | 1.39 | 4.51E−05 | 7.19E−04 |
| Neuroendocrine convertase | PCSK1 | P29120 | 0.93 | 0.39 | 1.47 | 6.89E−04 | 7.13E−03 |
| 1 | | | | | | | |
| Low-density lipoprotein | LRP10 | Q7Z4F1 | 0.93 | 0.37 | 1.49 | 1.15E−03 | 1.07E−02 |
| receptor-related protein 10 | | | | | | | |
| Guanine nucleotide | RABIF | P47224 | 0.93 | 0.51 | 1.35 | 1.36E−05 | 2.64E−04 |
| exchange factor MSS4 | | | | | | | |
| Hepatocyte growth factor | HGF | P14210 | 0.93 | 0.53 | 1.33 | 4.69E−06 | 1.05E−04 |
| Netrin-4 | NTN4 | Q9HB63 | 0.93 | 0.36 | 1.5 | 1.39E−03 | 1.24E−02 |
| Tenascin | TNC | P24821 | 0.93 | 0.6 | 1.25 | 2.38E−08 | 9.41E−07 |
| Inter-alpha-trypsin | ITIH3 | Q06033 | 0.93 | 0.49 | 1.38 | 4.23E−05 | 6.86E−04 |
| inhibitor heavy chain H3 | | | | | | | |
| Interleukin-1 receptor | IL1RN | P18510 | 0.92 | 0.5 | 1.35 | 2.15E−05 | 3.84E−04 |
| antagonist protein | | | | | | | |
| Cytokine receptor-like | CRLF1 | O75462 | 0.91 | 0.53 | 1.28 | 1.83E−06 | 4.62E−05 |
| factor 1 | | | | | | | |
| Stathmin | STMN1 | P16949 | 0.91 | 0.41 | 1.4 | 3.62E−04 | 4.37E−03 |
| D-dimer | FGA FGB | P02679\|P02675\|P02671 | 0.91 | 0.39 | 1.42 | 5.42E−04 | 5.99E−03 |
| | FGG | | | | | | |
| Desmocollin-2 | DSC2 | Q02487 | 0.9 | 0.38 | 1.43 | 7.01E−04 | 7.20E−03 |
| Ras-related protein Rab- | RAB3B | P20337 | 0.9 | 0.45 | 1.34 | 7.82E−05 | 1.15E−03 |
| 3B | | | | | | | |
| Copper transport protein | ATOX1 | O00244 | 0.9 | 0.51 | 1.28 | 6.91E−06 | 1.46E−04 |
| ATOX1 | | | | | | | |
| C5a anaphylatoxin | C5 | P01031 | 0.9 | 0.24 | 1.56 | 7.51E−03 | 4.54E−02 |
| Interleukin-18-binding | IL18BP | O95998 | 0.9 | 0.52 | 1.29 | 4.33E−06 | 9.84E−05 |
| protein | | | | | | | |
| Chymotrypsin-like elastase | CELA1 | Q9UNI1 | 0.9 | 0.55 | 1.26 | 7.59E−07 | 2.07E−05 |
| family member 1 | | | | | | | |
| Tumor necrosis factor | TNFRSF11B | O00300 | 0.9 | 0.51 | 1.28 | 5.29E−06 | 1.17E−04 |
| receptor superfamily | | | | | | | |
| member 11B | | | | | | | |
| Transmembrane protein | TMEM190 | Q8WZ59 | 0.89 | 0.57 | 1.21 | 5.51E−08 | 1.99E−06 |
| 190 | | | | | | | |
| Septin-11 | 43719 | Q9NVA2 | 0.89 | 0.33 | 1.44 | 1.81E−03 | 1.54E−02 |
| Secreted frizzled-related | SFRP4 | Q6FHJ7 | 0.89 | 0.44 | 1.33 | 8.78E−05 | 1.28E−03 |
| protein 4 | | | | | | | |
| Laminin subunit gamma-2 | LAMC2 | Q13753 | 0.89 | 0.5 | 1.28 | 6.59E−06 | 1.40E−04 |
| Programmed cell death | PDCD5 | O14737 | 0.88 | 0.51 | 1.25 | 3.56E−06 | 8.40E−05 |
| protein 5 | | | | | | | |
| Prefoldin subunit 2 | PFDN2 | Q9UHV9 | 0.88 | 0.34 | 1.42 | 1.52E−03 | 1.32E−02 |
| Alpha-1-antichymotrypsin | SERPINA3 | P07288\|P01011 | 0.88 | 0.46 | 1.3 | 4.34E−05 | 6.97E−04 |
| complex | | | | | | | |
| Atrial natriuretic factor | NPPA | P01160 | 0.88 | 0.42 | 1.35 | 1.81E−04 | 2.44E−03 |
| Vitrin | VIT | Q6UXI7 | 0.88 | 0.44 | 1.33 | 9.87E−05 | 1.42E−03 |
| Calcineurin B homologous | CHP1 | Q99653 | 0.87 | 0.48 | 1.26 | 1.21E−05 | 2.38E−04 |
| protein 1 | | | | | | | |
| Semaphorin-4A | SEMA4A | Q9H3S1 | 0.87 | 0.28 | 1.45 | 3.79E−03 | 2.66E−02 |
| Heat shock 70 kDa protein | HSPA1A | PODMV8 | 0.87 | 0.34 | 1.39 | 1.25E−03 | 1.13E−02 |
| 1A | | | | | | | |
| Apolipoprotein A-II | APOA2 | P02652 | 0.87 | 0.38 | 1.35 | 4.62E−04 | 5.30E−03 |
| Collagen alpha-1(XV) | COL15A1 | P39059 | 0.87 | 0.29 | 1.45 | 3.29E−03 | 2.42E−02 |
| chain | | | | | | | |
| Proto-oncogene tyrosine- | RET | P07949 | 0.86 | 0.47 | 1.25 | 1.59E−05 | 2.99E−04 |
| protein kinase receptor Ret | | | | | | | |
| Lysozyme C | LYZ | P61626 | 0.86 | 0.45 | 1.26 | 3.34E−05 | 5.58E−04 |
| SLAM family member 7 | SLAMF7 | Q9NQ25 | 0.86 | 0.6 | 1.11 | 3.14E−11 | 2.37E−09 |
| Transgelin | TAGLN | Q01995 | 0.85 | 0.4 | 1.3 | 1.98E−04 | 2.61E−03 |
| Chloride intracellular | CLIC2 | O15247 | 0.85 | 0.42 | 1.29 | 1.09E−04 | 1.56E−03 |
| channel protein 2 | | | | | | | |
| Fibroblast growth factor 23 | FGF23 | Q9GZV9 | 0.85 | 0.52 | 1.18 | 3.81E−07 | 1.11E−05 |
| Troponin T, cardiac | TNNT2 | P45379 | 0.85 | 0.48 | 1.22 | 6.38E−06 | 1.37E−04 |
| muscle | | | | | | | |
| Tolloid-like protein 1 | TLL1 | O43897 | 0.85 | 0.36 | 1.34 | 6.97E−04 | 7.17E−03 |
| DnaJ homolog subfamily | DNAJA4 | Q8WW22 | 0.85 | 0.41 | 1.29 | 1.73E−04 | 2.34E−03 |
| A member 4 | | | | | | | |
| Steroid receptor RNA | SRA1 | Q9HD15 | 0.84 | 0.46 | 1.23 | 1.68E−05 | 3.12E−04 |
| activator 1 | | | | | | | |
| N-acetylneuraminate lyase | NPL | Q9BXD5 | 0.84 | 0.4 | 1.28 | 1.87E−04 | 2.49E−03 |
| A disintegrin and | ADAMTS6 | Q9UKP5 | 0.84 | 0.34 | 1.34 | 1.01E−03 | 9.71E−03 |
| metalloproteinase with | | | | | | | |
| thrombospondin motifs 6 | | | | | | | |
| Ectonucleoside | ENTPD1 | P49961 | 0.84 | 0.36 | 1.31 | 5.19E−04 | 5.82E−03 |
| triphosphate | | | | | | | |
| diphosphohydrolase 1 | | | | | | | |
| Trafficking protein particle | TRAPPC3 | O43617 | 0.83 | 0.45 | 1.21 | 1.78E−05 | 3.26E−04 |
| complex subunit 3 | | | | | | | |
| Kallikrein-14 | KLK14 | Q9P0G3 | 0.83 | 0.48 | 1.17 | 2.49E−06 | 6.04E−05 |
| Tyrosine-protein kinase | TIE1 | P35590 | 0.83 | 0.24 | 1.43 | 6.25E−03 | 3.98E−02 |
| receptor Tie-1, soluble | | | | | | | |
| CMRF35-like molecule 6 | CD300C | Q08708 | 0.83 | 0.32 | 1.34 | 1.38E−03 | 1.24E−02 |
| Glucokinase regulatory | GCKR | Q14397 | 0.83 | 0.27 | 1.39 | 3.70E−03 | 2.63E−02 |
| protein | | | | | | | |
| Heat shock 70 kDa protein | HSPA13 | P48723 | 0.82 | 0.23 | 1.4 | 6.30E−03 | 3.99E−02 |
| 13 | | | | | | | |
| Rho-related GTP-binding | RHOB | P62745 | 0.82 | 0.22 | 1.41 | 7.59E−03 | 4.57E−02 |
| protein RhoB | | | | | | | |
| Acidic leucine-rich nuclear | ANP32B | Q92688 | 0.82 | 0.29 | 1.36 | 2.61E−03 | 2.04E−02 |
| phosphoprotein 32 family | | | | | | | |
| member B | | | | | | | |
| Tissue Factor | F3 | P13726 | 0.82 | 0.38 | 1.26 | 2.68E−04 | 3.39E−03 |
| Leukocyte | LILRB2 | Q8N423 | 0.82 | 0.24 | 1.4 | 5.70E−03 | 3.70E−02 |
| immunoglobulin-like | | | | | | | |
| receptor subfamily B | | | | | | | |
| member 2 | | | | | | | |
| Protein kinase C gamma | PRKCG | P05129 | 0.82 | 0.28 | 1.36 | 2.85E−03 | 2.15E−02 |
| type | | | | | | | |
| Signaling lymphocytic | SLAMF1 | Q13291 | 0.82 | 0.39 | 1.25 | 1.86E−04 | 2.49E−03 |
| activation molecule | | | | | | | |
| Calcium/calmodulin- | CAMKK1 | Q8N5S9 | 0.81 | 0.23 | 1.39 | 5.90E−03 | 3.80E−02 |
| dependent protein kinase | | | | | | | |
| kinase 1 | | | | | | | |
| Cerebral dopamine | CDNF | Q49AH0 | 0.81 | 0.27 | 1.36 | 3.28E−03 | 2.42E−02 |
| neurotrophic factor | | | | | | | |
| Microfibril-associated | MFAP4 | P55083 | 0.81 | 0.43 | 1.19 | 2.86E−05 | 4.90E−04 |
| glycoprotein 4 | | | | | | | |
| Secretoglobin family 3A | SCGB3A1 | Q96QR1 | 0.81 | 0.55 | 1.07 | 7.26E−10 | 4.16E−08 |
| member 1 | | | | | | | |
| Four-jointed box protein 1 | FJX1 | Q86VR8 | 0.81 | 0.36 | 1.26 | 4.04E−04 | 4.77E−03 |
| Kallikrein-13 | KLK13 | Q9UKR3 | 0.8 | 0.47 | 1.14 | 1.99E−06 | 4.95E−05 |
| Switch-associated protein | SWAP70 | Q9UH65 | 0.8 | 0.23 | 1.37 | 6.07E−03 | 3.90E−02 |
| 70 | | | | | | | |
| Macrophage colony- | CSF1R | P07333 | 0.8 | 0.37 | 1.23 | 2.42E−04 | 3.13E−03 |
| stimulating factor 1 | | | | | | | |
| receptor | | | | | | | |
| Natriuretic peptides B | NPPB | P16860 | 0.8 | 0.43 | 1.17 | 2.50E−05 | 4.36E−04 |
| Dual specificity mitogen- | MAP2K1 | Q02750 | 0.8 | 0.26 | 1.34 | 3.55E−03 | 2.56E−02 |
| activated protein kinase | | | | | | | |
| kinase 1 | | | | | | | |
| Periostin | POSTN | Q15063 | 0.8 | 0.33 | 1.26 | 8.31E−04 | 8.26E−03 |
| Cerebellin-4 | CBLN4 | Q9NTU7 | 0.8 | 0.32 | 1.28 | 1.11E−03 | 1.04E−02 |
| Oncoprotein-induced | OIT3 | Q8WWZ8 | 0.8 | 0.38 | 1.22 | 1.74E−04 | 2.34E−03 |
| transcript 3 protein | | | | | | | |
| Receptor-interacting | RIPK2 | O43353 | 0.8 | 0.32 | 1.28 | 1.18E−03 | 1.09E−02 |
| serine/threonine-protein | | | | | | | |
| kinase 2 | | | | | | | |
| Nidogen-1 | NID1 | P14543 | 0.79 | 0.3 | 1.28 | 1.51E−03 | 1.32E−02 |
| C-X-C motif chemokine | CXCL10 | P02778 | 0.79 | 0.47 | 1.1 | 9.80E−07 | 2.55E−05 |
| 10 | | | | | | | |
| Interleukin-18 | IL18 | Q14116 | 0.79 | 0.41 | 1.18 | 5.13E−05 | 7.94E−04 |
| 26S proteasome non- | PSMD4 | P55036 | 0.78 | 0.34 | 1.22 | 5.03E−04 | 5.68E−03 |
| ATPase regulatory subunit | | | | | | | |
| 4 | | | | | | | |
| Holo-Transcobalamin-2 | TCN2 | P20062 | 0.78 | 0.24 | 1.33 | 4.96E−03 | 3.34E−02 |
| GTPase NRas | NRAS | P01111 | 0.77 | 0.28 | 1.27 | 2.08E−03 | 1.72E−02 |
| Alpha-parvin | PARVA | Q9NVD7 | 0.77 | 0.27 | 1.26 | 2.33E−03 | 1.87E−02 |
| Interleukin-12 subunit beta | IL12B | P29460 | 0.77 | 0.4 | 1.13 | 3.99E−05 | 6.53E−04 |
| Gastric inhibitory | GIP | P09681 | 0.77 | 0.49 | 1.05 | 4.93E−08 | 1.82E−06 |
| polypeptide | | | | | | | |
| SLAM family member 6 | SLAMF6 | Q96DU3 | 0.77 | 0.3 | 1.25 | 1.33E−03 | 1.20E−02 |
| Protein ABHD14A | ABHD14A | Q9BUJ0 | 0.77 | 0.39 | 1.15 | 7.18E−05 | 1.07E−03 |
| Protocadherin beta-10 | PCDHB10 | Q9UN67 | 0.77 | 0.3 | 1.24 | 1.33E−03 | 1.20E−02 |
| DnaJ homolog subfamily | DNAJB4 | Q9UDY4 | 0.76 | 0.47 | 1.06 | 5.49E−07 | 1.55E−05 |
| B member 4 | | | | | | | |
| Histidine triad nucleotide- | HINT1 | P49773 | 0.76 | 0.32 | 1.2 | 7.50E−04 | 7.62E−03 |
| binding protein 1 | | | | | | | |
| Protocadherin gamma-A10 | PCDHGA10 | Q9Y5H3 | 0.76 | 0.39 | 1.13 | 6.14E−05 | 9.32E−04 |
| Y-box-binding protein 2 | YBX2 | Q9Y2T7 | 0.76 | 0.34 | 1.18 | 3.47E−04 | 4.21E−03 |
| Ubiquitin-conjugating | UBE2G2 | P60604 | 0.76 | 0.23 | 1.28 | 4.56E−03 | 3.11E−02 |
| enzyme E2 G2 | | | | | | | |
| CD48 antigen | CD48 | P09326 | 0.75 | 0.34 | 1.16 | 3.43E−04 | 4.19E−03 |
| Signal-regulatory protein | SIRPB2 | Q5JXA9 | 0.75 | 0.25 | 1.25 | 3.48E−03 | 2.53E−02 |
| beta-2 | | | | | | | |
| Hepatoma-derived growth | HDGF | P51858 | 0.75 | 0.36 | 1.14 | 1.66E−04 | 2.25E−03 |
| factor | | | | | | | |
| Protein lin-7 homolog B | LIN7B | Q9HAP6 | 0.74 | 0.28 | 1.19 | 1.51E−03 | 1.32E−02 |
| 60S ribosomal protein L12 | RPL12 | P30050 | 0.74 | 0.42 | 1.06 | 6.04E−06 | 1.30E−04 |
| Midkine | MDK | P21741 | 0.74 | 0.36 | 1.13 | 1.63E−04 | 2.21E−03 |
| Ribonuclease pancreatic | RNASE1 | P07998 | 0.74 | 0.49 | 0.99 | 6.50E−09 | 2.81E−07 |
| BMP and activin | BAMBI | Q13145 | 0.74 | 0.26 | 1.22 | 2.59E−03 | 2.02E−02 |
| membrane-bound inhibitor | | | | | | | |
| homolog: Extracellular | | | | | | | |
| domain | | | | | | | |
| Pleiotrophin | PTN | P21246 | 0.73 | 0.26 | 1.19 | 2.17E−03 | 1.77E−02 |
| Fatty acid-binding protein, | FABP3 | P05413 | 0.73 | 0.35 | 1.11 | 1.51E−04 | 2.06E−03 |
| heart | | | | | | | |
| Putative chondrosarcoma- | CSAG1 | Q6PB30 | 0.73 | 0.2 | 1.27 | 7.25E−03 | 4.42E−02 |
| associated gene 1 protein | | | | | | | |
| Semaphorin-4D | SEMA4D | Q92854 | 0.73 | 0.26 | 1.2 | 2.48E−03 | 1.96E−02 |
| Cathepsin H | CTSH | P09668 | 0.73 | 0.36 | 1.11 | 1.35E−04 | 1.87E−03 |
| Polypeptide N- | GALNT16 | Q8N428 | 0.73 | 0.3 | 1.16 | 9.75E−04 | 9.41E−03 |
| acetylgalactosaminyltransferase | | | | | | | |
| 16 | | | | | | | |
| Trinucleotide repeat- | TNRC6B | Q9UPQ9 | 0.73 | 0.25 | 1.21 | 3.10E−03 | 2.31E−02 |
| containing gene 6B protein | | | | | | | |
| Transformer-2 protein | TRA2B | P62995 | 0.72 | 0.37 | 1.08 | 6.08E−05 | 9.28E−04 |
| homolog beta | | | | | | | |
| Leucine-rich repeat | FLRT2 | O43155 | 0.72 | 0.24 | 1.19 | 3.01E−03 | 2.25E−02 |
| transmembrane protein | | | | | | | |
| FLRT2 | | | | | | | |
| Guanylate-binding protein | GBP1 | P32455 | 0.72 | 0.35 | 1.09 | 1.29E−04 | 1.80E−03 |
| 1 | | | | | | | |
| Tenascin | TNC | P24821 | 0.72 | 0.19 | 1.26 | 8.16E−03 | 4.81E−02 |
| HLA class II | CD74 | P04233 | 0.72 | 0.25 | 1.19 | 2.56E−03 | 2.01E−02 |
| histocompatibility antigen | | | | | | | |
| gamma chain | | | | | | | |
| DnaJ homolog subfamily | DNAJB6 | O75190 | 0.71 | 0.23 | 1.19 | 4.01E−03 | 2.78E−02 |
| B member 6 | | | | | | | |
| Envoplakin | EVPL | Q92817 | 0.71 | 0.25 | 1.17 | 2.68E−03 | 2.07E−02 |
| Vascular endothelial | FIGF | O43915 | 0.71 | 0.37 | 1.06 | 5.84E−05 | 8.97E−04 |
| growth factor D | | | | | | | |
| Poly(ADP-ribose) | ADPRHL2 | Q9NX46 | 0.71 | 0.37 | 1.05 | 4.36E−05 | 6.98E−04 |
| glycohydrolase ARH3 | | | | | | | |
| Serum amyloid A-2 | SAA2 | PODJI9 | 0.71 | 0.5 | 0.93 | 1.21E−10 | 8.00E−09 |
| protein | | | | | | | |
| Urokinase plasminogen | PLAUR | Q03405 | 0.71 | 0.25 | 1.16 | 2.20E−03 | 1.79E−02 |
| activator surface receptor | | | | | | | |
| Procollagen C- | PCOLCE | Q15113 | 0.7 | 0.25 | 1.14 | 2.07E−03 | 1.71E−02 |
| endopeptidase enhancer 1 | | | | | | | |
| Fructose-2,6- | TIGAR | Q9NQ88 | 0.7 | 0.22 | 1.17 | 3.90E−03 | 2.73E−02 |
| bisphosphatase TIGAR | | | | | | | |
| RNA-binding protein EWS | EWSR1 | Q01844 | 0.7 | 0.24 | 1.17 | 3.20E−03 | 2.37E−02 |
| Interleukin-18 receptor 1 | IL18R1 | Q13478 | 0.7 | 0.31 | 1.09 | 3.87E−04 | 4.60E−03 |
| Heat shock 70 kDa protein | HSPA1A | P0DMV8 | 0.7 | 0.25 | 1.16 | 2.25E−03 | 1.82E−02 |
| 1A | | | | | | | |
| Folate receptor alpha | FOLR1 | P15328 | 0.7 | 0.29 | 1.1 | 8.39E−04 | 8.30E−03 |
| Interleukin-17 receptor C | IL17RC | Q8NAC3 | 0.7 | 0.31 | 1.1 | 5.19E−04 | 5.82E−03 |
| Riboflavin kinase | RFK | Q969G6 | 0.69 | 0.18 | 1.2 | 8.06E−03 | 4.79E−02 |
| Progesterone receptor | PGR | P06401 | 0.69 | 0.3 | 1.07 | 4.37E−04 | 5.04E−03 |
| Aldose reductase | AKR1B1 | P15121 | 0.69 | 0.28 | 1.1 | 1.07E−03 | 1.01E−02 |
| Adhesion G-protein | GPR56 | Q9Y653 | 0.69 | 0.29 | 1.08 | 6.73E−04 | 6.99E−03 |
| coupled receptor G1 | | | | | | | |
| Heat shock 70 kDa protein | PCSK2 | P0DMV8 | 0.69 | 0.19 | 1.18 | 6.27E−03 | 3.98E−02 |
| 1A | | | | | | | |
| Interleukin-23 | IL12B | Q9NPF7\|P29460 | 0.68 | 0.28 | 1.08 | 8.07E−04 | 8.07E−03 |
| | IL23A | | | | | | |
| Ubiquitin-conjugating | UBE2E1 | P51965 | 0.68 | 0.35 | 1 | 4.20E−05 | 6.84E−04 |
| enzyme E2 E1 | | | | | | | |
| Nicotinamide N- | NNMT | P40261 | 0.68 | 0.36 | 1 | 3.22E−05 | 5.46E−04 |
| methyltransferase | | | | | | | |
| Complement receptor type | CR1 | P17927 | 0.68 | 0.19 | 1.17 | 6.71E−03 | 4.18E−02 |
| 1 | | | | | | | |
| Asialoglycoprotein | ASGR1 | P07306 | 0.68 | 0.22 | 1.15 | 3.91E−03 | 2.73E−02 |
| receptor 1 | | | | | | | |
| Annexin A2 | ANXA2 | P07355 | 0.67 | 0.24 | 1.09 | 2.13E−03 | 1.74E−02 |
| Membrane-associated | MAGI2 | Q86UL8 | 0.67 | 0.27 | 1.06 | 9.20E−04 | 8.96E−03 |
| guanylate kinase, WW and | | | | | | | |
| PDZ domain-containing | | | | | | | |
| protein 2 | | | | | | | |
| Left-right determination | LEFTY2 | O00292 | 0.67 | 0.22 | 1.12 | 3.66E−03 | 2.61E−02 |
| factor 2 | | | | | | | |
| 14 kDa phosphohistidine | PHPT1 | Q9NRX4 | 0.67 | 0.26 | 1.08 | 1.26E−03 | 1.15E−02 |
| phosphatase | | | | | | | |
| Peptidyl-prolyl cis-trans | FKBP4 | Q02790 | 0.67 | 0.29 | 1.05 | 5.55E−04 | 6.08E−03 |
| isomerase FKBP4 | | | | | | | |
| Heparanase | HPSE | Q9Y251 | 0.67 | 0.27 | 1.06 | 9.42E−04 | 9.13E−03 |
| Protein FAM171A1 | FAM171A1 | Q5VUB5 | 0.67 | 0.28 | 1.06 | 7.84E−04 | 7.88E−03 |
| Protein disulfide-isomerase | TMX3 | Q96JJ7 | 0.67 | 0.2 | 1.14 | 4.99E−03 | 3.35E−02 |
| TMX3 | | | | | | | |
| Thrombopoietin | THPO | P40225 | 0.67 | 0.31 | 1.02 | 2.47E−04 | 3.18E−03 |
| Thioredoxin domain- | TXNDC15 | Q96J42 | 0.67 | 0.29 | 1.06 | 5.25E−04 | 5.87E−03 |
| containing protein 15 | | | | | | | |
| SLP adapter and CSK- | SCIMP | Q6UWF3 | 0.66 | 0.23 | 1.08 | 2.54E−03 | 2.00E−02 |
| interacting membrane | | | | | | | |
| protein | | | | | | | |
| Alpha-galactosidase A | GLA | P06280 | 0.66 | 0.24 | 1.09 | 2.19E−03 | 1.78E−02 |
| StAR-related lipid transfer | STARD5 | Q9NSY2 | 0.66 | 0.28 | 1.04 | 6.93E−04 | 7.16E−03 |
| protein 5 | | | | | | | |
| Microtubule-associated | MAP1LC3A | Q9H492 | 0.66 | 0.28 | 1.04 | 7.58E−04 | 7.67E−03 |
| proteins 1A/1B light chain | | | | | | | |
| 3A | | | | | | | |
| Glutaredoxin-1 | GLRX | P35754 | 0.66 | 0.17 | 1.16 | 8.23E−03 | 4.84E−02 |
| Insulin | INS | P01308 | 0.66 | 0.37 | 0.96 | 8.04E−06 | 1.65E−04 |
| BH3-interacting domain | BID | P55957 | 0.66 | 0.28 | 1.04 | 6.62E−04 | 6.89E−03 |
| death agonist | | | | | | | |
| Complement C3d fragment | C3 | P01024 | 0.66 | 0.25 | 1.08 | 1.90E−03 | 1.59E−02 |
| Peptidyl-prolyl cis-trans | FKBP7 | Q9Y680 | 0.66 | 0.32 | 1 | 1.55E−04 | 2.12E−03 |
| isomerase FKBP7 | | | | | | | |
| 78 kDa glucose-regulated | HSPA5 | P11021 | 0.65 | 0.25 | 1.04 | 1.46E−03 | 1.28E−02 |
| protein | | | | | | | |
| Leukotriene A-4 hydrolase | LTA4H | P09960 | 0.65 | 0.34 | 0.96 | 3.31E−05 | 5.55E−04 |
| C-reactive protein | CRP | P02741 | 0.65 | 0.48 | 0.82 | 3.75E−14 | 5.26E−12 |
| Uroporphyrinogen-III | UROS | P10746 | 0.64 | 0.26 | 1.03 | 9.82E−04 | 9.46E−03 |
| synthase | | | | | | | |
| NACHT, LRR and PYD | NLRP1 | Q9C000 | 0.64 | 0.25 | 1.03 | 1.42E−03 | 1.26E−02 |
| domains-containing | | | | | | | |
| protein 1 | | | | | | | |
| Thiosulfate | TST | Q16762 | 0.64 | 0.37 | 0.91 | 3.22E−06 | 7.70E−05 |
| sulfurtransferase | | | | | | | |
| Inhibin beta B chain | INHBB | P09529 | 0.64 | 0.3 | 0.97 | 1.86E−04 | 2.49E−03 |
| Fatty acid-binding protein, | FABP4 | P15090 | 0.64 | 0.32 | 0.96 | 9.87E−05 | 1.42E−03 |
| adipocyte | | | | | | | |
| Syntaxin-12 | STX12 | Q86Y82 | 0.63 | 0.29 | 0.97 | 2.42E−04 | 3.13E−03 |
| Malignant T-cell-amplified | MCTS1 | Q9ULC4 | 0.63 | 0.27 | 1 | 6.26E−04 | 6.64E−03 |
| sequence 1 | | | | | | | |
| Hyaluronan and | HAPLN1 | P10915 | 0.63 | 0.22 | 1.05 | 2.80E−03 | 2.13E−02 |
| proteoglycan link protein 1 | | | | | | | |
| Palmitoyl-protein | PPT1 | P50897 | 0.63 | 0.25 | 1.02 | 1.41E−03 | 1.25E−02 |
| thioesterase 1 | | | | | | | |
| Egl nine homolog 1 | EGLN1 | Q9GZT9 | 0.63 | 0.16 | 1.1 | 8.16E−03 | 4.81E−02 |
| E3 ubiquitin-protein ligase | ZNRF3 | Q9ULT6 | 0.62 | 0.22 | 1.02 | 2.20E−03 | 1.79E−02 |
| ZNRF3 | | | | | | | |
| Protein phosphatase 1A | PPM1A | P35813 | 0.62 | 0.21 | 1.02 | 2.91E−03 | 2.19E−02 |
| Ras-related protein Rab-14 | RAB14 | P61106 | 0.62 | 0.24 | 1 | 1.42E−03 | 1.26E−02 |
| Netrin receptor UNC5B | UNC5B | Q8IZJ1 | 0.62 | 0.18 | 1.05 | 5.30E−03 | 3.51E−02 |
| Tripeptidyl-peptidase 1 | TPP1 | O14773 | 0.62 | 0.17 | 1.06 | 6.46E−03 | 4.06E−02 |
| Protein S100-Z | S100Z | Q8WXG8 | 0.62 | 0.27 | 0.98 | 6.42E−04 | 6.77E−03 |
| tRNA pseudouridine | PUS1 | Q9Y606 | 0.61 | 0.17 | 1.06 | 7.14E−03 | 4.37E−02 |
| synthase A, mitochondrial | | | | | | | |
| Ubiquitin-like protein | ISG15 | P05161 | 0.61 | 0.35 | 0.86 | 2.94E−06 | 7.10E−05 |
| ISG15 | | | | | | | |
| Uridine phosphorylase 1 | UPP1 | Q16831 | 0.61 | 0.17 | 1.04 | 5.91E−03 | 3.81E−02 |
| Intercellular adhesion | ICAM4 | Q14773 | 0.61 | 0.27 | 0.95 | 4.71E−04 | 5.38E−03 |
| molecule 4 | | | | | | | |
| Fas apoptotic inhibitory | FAIM3 | O60667 | 0.61 | 0.31 | 0.91 | 7.07E−05 | 1.06E−03 |
| molecule 3 | | | | | | | |
| Membrane-associated | PGRMC2 | O15173 | 0.6 | 0.24 | 0.97 | 1.14E−03 | 1.07E−02 |
| progesterone receptor | | | | | | | |
| component 2 | | | | | | | |
| Regenerating islet-derived | REG4 | Q9BYZ8 | 0.6 | 0.24 | 0.96 | 1.11E−03 | 1.04E−02 |
| protein 4 | | | | | | | |
| R-spondin-1 | RSPO1 | Q2MKA7 | 0.6 | 0.16 | 1.04 | 8.12E−03 | 4.81E−02 |
| Scavenger receptor | CD163 | Q86VB7 | 0.6 | 0.22 | 0.98 | 1.90E−03 | 1.59E−02 |
| cysteine-rich type 1 protein | | | | | | | |
| M130 | | | | | | | |
| Disintegrin and | ADAM11 | O75078 | 0.6 | 0.17 | 1.03 | 5.71E−03 | 3.70E−02 |
| metalloproteinase domain- | | | | | | | |
| containing protein 11 | | | | | | | |
| Lamina-associated | TMPO | P42167 | 0.6 | 0.25 | 0.95 | 6.81E−04 | 7.07E−03 |
| polypeptide 2, isoforms | | | | | | | |
| beta/gamma | | | | | | | |
| Sialate O-acetylesterase | SIAE | Q9HAT2 | 0.6 | 0.26 | 0.95 | 5.96E−04 | 6.38E−03 |
| Tudor-interacting repair | NUDT16L1 | Q9BRJ7 | 0.59 | 0.27 | 0.92 | 3.96E−04 | 4.69E−03 |
| regulator protein | | | | | | | |
| SH3 and multiple ankyrin | SHANK3 | Q9BYB0 | 0.59 | 0.28 | 0.91 | 2.55E−04 | 3.26E−03 |
| repeat domains protein 3 | | | | | | | |
| Oncostatin-M | OSM | P13725 | 0.59 | 0.19 | 0.99 | 3.75E−03 | 2.65E−02 |
| Astrocytic phosphoprotein | PEA15 | Q15121 | 0.59 | 0.15 | 1.02 | 7.97E−03 | 4.75E−02 |
| PEA-15 | | | | | | | |
| Eukaryotic translation | EIF5 | P55010 | 0.59 | 0.21 | 0.97 | 2.27E−03 | 1.83E−02 |
| initiation factor 5 | | | | | | | |
| Galectin-3-binding protein | LGALS3BP | Q08380 | 0.59 | 0.29 | 0.88 | 9.56E−05 | 1.39E−03 |
| Carboxypeptidase Z | CPZ | Q66K79 | 0.59 | 0.17 | 1.01 | 5.51E−03 | 3.61E−02 |
| Cholecystokinin | CCK | P06307 | 0.59 | 0.2 | 0.97 | 2.64E−03 | 2.06E−02 |
| Protein eva-1 homolog B | EVA1B | Q9NVM1 | 0.59 | 0.19 | 0.99 | 4.13E−03 | 2.85E−02 |
| Cysteine and glycine-rich | CSRP3 | P50461 | 0.59 | 0.18 | 1.01 | 5.34E−03 | 3.52E−02 |
| protein 3 | | | | | | | |
| Trefoil factor 2 | TFF2 | Q03403 | 0.59 | 0.27 | 0.9 | 2.43E−04 | 3.14E−03 |
| Peptide YY | PYY | P10082 | 0.58 | 0.23 | 0.94 | 1.22E−03 | 1.12E−02 |
| LEM domain-containing | LEMD1 | Q68G75 | 0.58 | 0.19 | 0.96 | 3.38E−03 | 2.47E−02 |
| protein 1 | | | | | | | |
| Stromelysin-2 | MMP10 | P09238 | 0.58 | 0.21 | 0.94 | 1.85E−03 | 1.56E−02 |
| Golgi membrane protein 1 | GOLM1 | Q8NBJ4 | 0.58 | 0.28 | 0.88 | 1.29E−04 | 1.80E−03 |
| Regenerating islet-derived | REG3A | Q06141 | 0.57 | 0.31 | 0.83 | 1.49E−05 | 2.84E−04 |
| protein 3-alpha | | | | | | | |
| Fibroblast growth factor- | FGFBP1 | Q14512 | 0.57 | 0.21 | 0.92 | 1.79E−03 | 1.53E−02 |
| binding protein 1 | | | | | | | |
| High affinity cGMP- | PDE9A | O76083 | 0.57 | 0.23 | 0.92 | 1.23E−03 | 1.12E−02 |
| specific 3′,5′-cyclic | | | | | | | |
| phosphodiesterase 9A | | | | | | | |
| Soluble calcium-activated | CANT1 | Q8WVQ1 | 0.57 | 0.24 | 0.9 | 6.58E−04 | 6.89E−03 |
| nucleotidase 1 | | | | | | | |
| Receptor activity- | RAMP1 | O60894 | 0.56 | 0.17 | 0.94 | 4.46E−03 | 3.06E−02 |
| modifying protein 1 | | | | | | | |
| C-C motif chemokine 17 | CCL17 | Q92583 | 0.56 | 0.28 | 0.85 | 1.11E−04 | 1.58E−03 |
| Receptor-type tyrosine- | PTPRU | Q92729 | 0.56 | 0.15 | 0.96 | 7.86E−03 | 4.70E−02 |
| protein phosphatase U | | | | | | | |
| Prokineticin-2 | PROK2 | Q9HC23 | 0.55 | 0.3 | 0.81 | 2.27E−05 | 4.03E−04 |
| T-cell surface glycoprotein | CD8A | P01732 | 0.55 | 0.21 | 0.89 | 1.51E−03 | 1.32E−02 |
| CD8 alpha chain | | | | | | | |
| Selenoprotein W | SEPW1 | P63302 | 0.55 | 0.19 | 0.9 | 2.36E−03 | 1.88E−02 |
| C-C motif chemokine 28 | CCL28 | Q9NRJ3 | 0.55 | 0.24 | 0.86 | 5.29E−04 | 5.88E−03 |
| C-X-C motif chemokine | CXCL11 | 014625 | 0.55 | 0.35 | 0.76 | 1.45E−07 | 4.79E−06 |
| 11 | | | | | | | |
| T-cell surface glycoprotein | CD5 | P06127 | 0.55 | 0.19 | 0.92 | 3.08E−03 | 2.30E−02 |
| CD5: Extracellular domain | | | | | | | |
| DnaJ homolog subfamily | DNAJB12 | Q9NXW2 | 0.55 | 0.25 | 0.84 | 2.92E−04 | 3.65E−03 |
| B member 12 | | | | | | | |
| Solute carrier organic | SLCO5A1 | Q9H2Y9 | 0.54 | 0.14 | 0.94 | 7.91E−03 | 4.73E−02 |
| anion transporter family | | | | | | | |
| member 5A1 | | | | | | | |
| Protein NDRG3 | NDRG3 | Q9UGV2 | 0.54 | 0.19 | 0.9 | 2.67E−03 | 2.07E−02 |
| Transcription factor MafG | MAFG | O15525 | 0.54 | 0.14 | 0.94 | 8.50E−03 | 4.97E−02 |
| C-C motif chemokine 25 | CCL25 | O15444 | 0.54 | 0.29 | 0.79 | 2.68E−05 | 4.65E−04 |
| Neutrophil gelatinase- | LCN2 | P80188 | 0.54 | 0.17 | 0.91 | 3.96E−03 | 2.75E−02 |
| associated lipocalin | | | | | | | |
| Eotaxin | CCL11 | P51671 | 0.54 | 0.15 | 0.92 | 7.17E−03 | 4.39E−02 |
| Protein S100-A12 | S100A12 | P80511 | 0.54 | 0.2 | 0.88 | 1.71E−03 | 1.47E−02 |
| Gastrokine-2 | GKN2 | Q86XP6 | 0.54 | 0.25 | 0.83 | 2.48E−04 | 3.18E−03 |
| Acylphosphatase-2 | ACYP2 | P14621 | 0.53 | 0.19 | 0.86 | 1.93E−03 | 1.61E−02 |
| Thioredoxin reductase 1, | TXNRD1 | Q16881 | 0.53 | 0.23 | 0.84 | 5.09E−04 | 5.73E−03 |
| cytoplasmic | | | | | | | |
| Platelet-activating factor | PAFAH1B2 | P68402 | 0.53 | 0.21 | 0.85 | 1.23E−03 | 1.12E−02 |
| acetylhydrolase IB subunit | | | | | | | |
| beta | | | | | | | |
| CD5 antigen-like | CD5L | O43866 | 0.53 | 0.25 | 0.8 | 1.93E−04 | 2.55E−03 |
| T-lymphocyte surface | LY9 | Q9HBG7 | 0.53 | 0.14 | 0.92 | 7.44E−03 | 4.51E−02 |
| antigen Ly-9 | | | | | | | |
| Cystatin-D | CST5 | P28325 | 0.53 | 0.31 | 0.76 | 4.69E−06 | 1.05E−04 |
| Lactoperoxidase | LPO | P22079 | 0.53 | 0.21 | 0.86 | 1.14E−03 | 1.07E−02 |
| Cartilage intermediate | CILP | O75339 | 0.53 | 0.22 | 0.85 | 9.74E−04 | 9.41E−03 |
| layer protein 1 | | | | | | | |
| Probable inactive | RNASE13 | Q5GAN3 | 0.53 | 0.15 | 0.9 | 5.93E−03 | 3.82E−02 |
| ribonuclease-like protein | | | | | | | |
| 13 | | | | | | | |
| Membrane-associated | PGRMC1 | O00264 | 0.53 | 0.16 | 0.9 | 5.07E−03 | 3.39E−02 |
| progesterone receptor | | | | | | | |
| component 1 | | | | | | | |
| Carbonic anhydrase 5A, | CASA | P35218 | 0.53 | 0.17 | 0.9 | 4.57E−03 | 3.12E−02 |
| mitochondrial | | | | | | | |
| Cysteine-rich secretory | CRISP2 | P16562 | 0.53 | 0.18 | 0.89 | 3.44E−03 | 2.50E−02 |
| protein 2 | | | | | | | |
| DNA repair protein | XRCC4 | Q13426 | 0.53 | 0.14 | 0.92 | 8.29E−03 | 4.87E−02 |
| XRCC4 | | | | | | | |
| UV excision repair protein | RAD23A | P54725 | 0.52 | 0.2 | 0.84 | 1.47E−03 | 1.29E−02 |
| RAD23 homolog A | | | | | | | |
| 26S proteasome non- | PSMD9 | O00233 | 0.52 | 0.15 | 0.88 | 5.66E−03 | 3.69E−02 |
| ATPase regulatory subunit | | | | | | | |
| 9 | | | | | | | |
| Serum amyloid A-1 | SAA1 | P0DJI8 | 0.52 | 0.37 | 0.68 | 6.65E−11 | 4.60E−09 |
| protein | | | | | | | |
| Triggering receptor | TREM2 | Q9NZC2 | 0.52 | 0.23 | 0.81 | 4.93E−04 | 5.59E−03 |
| expressed on myeloid cells | | | | | | | |
| 2 | | | | | | | |
| Transcription elongation | TCEA1 | P23193 | 0.52 | 0.19 | 0.85 | 1.87E−03 | 1.57E−02 |
| factor A protein 1 | | | | | | | |
| CD40 ligand | CD40LG | P29965 | 0.52 | 0.18 | 0.87 | 2.72E−03 | 2.09E−02 |
| Troponin I, fast skeletal | TNNI2 | P48788 | 0.52 | 0.29 | 0.75 | 6.91E−06 | 1.46E−04 |
| muscle | | | | | | | |
| Defensin-5 | DEFA5 | Q01523 | 0.51 | 0.21 | 0.8 | 8.36E−04 | 8.30E−03 |
| WW domain-binding | WBP2 | Q969T9 | 0.51 | 0.14 | 0.88 | 6.90E−03 | 4.27E−02 |
| protein 2 | | | | | | | |
| Kunitz-type protease | SPINT2 | O43291 | 0.51 | 0.14 | 0.89 | 6.96E−03 | 4.29E−02 |
| inhibitor 2 | | | | | | | |
| Alpha-1,3-mannosyl- | MGAT4C | Q9UBM8 | 0.51 | 0.15 | 0.88 | 5.35E−03 | 3.53E−02 |
| glycoprotein 4-beta-N- | | | | | | | |
| acetylglucosaminyltransferase | | | | | | | |
| C | | | | | | | |
| SLAM family member 8 | SLAMF8 | Q9P0V8 | 0.51 | 0.23 | 0.79 | 4.46E−04 | 5.12E−03 |
| Peptidyl-prolyl cis-trans | FKBP14 | Q9NWM8 | 0.51 | 0.21 | 0.8 | 7.89E−04 | 7.92E−03 |
| isomerase FKBP14 | | | | | | | |
| Killer cell lectin-like | KLRB1 | Q12918 | 0.5 | 0.15 | 0.85 | 5.27E−03 | 3.49E−02 |
| receptor subfamily B | | | | | | | |
| member 1 | | | | | | | |
| Collagen type IV alpha-3- | COLAA3BP | Q9Y5P4 | 0.5 | 0.23 | 0.76 | 2.73E−04 | 3.44E−03 |
| binding protein: StAR- | | | | | | | |
| related lipid-transfer | | | | | | | |
| domain, isoform 2 | | | | | | | |
| Isopentenyl-diphosphate | IDI1 | Q13907 | 0.5 | 0.22 | 0.78 | 4.37E−04 | 5.04E−03 |
| Delta-isomerase 1 | | | | | | | |
| Myeloid cell surface | CD33 | P20138 | 0.5 | 0.22 | 0.79 | 5.84E−04 | 6.30E−03 |
| antigen CD33 | | | | | | | |
| Granulysin | GNLY | P22749 | 0.5 | 0.19 | 0.82 | 1.87E−03 | 1.57E−02 |
| Peroxiredoxin-1 | PRDX1 | Q06830 | 0.5 | 0.13 | 0.86 | 7.27E−03 | 4.43E−02 |
| Interleukin-6 | IL6 | P05231 | 0.5 | 0.21 | 0.78 | 6.03E−04 | 6.45E−03 |
| Cathepsin D | CTSD | P07339 | 0.5 | 0.2 | 0.8 | 1.10E−03 | 1.04E−02 |
| Lymphocyte antigen 6 | LY6G6C | O95867 | 0.5 | 0.16 | 0.83 | 3.92E−03 | 2.73E−02 |
| complex locus protein G6c | | | | | | | |
| Stathmin-2 | STMN2 | Q93045 | 0.49 | 0.17 | 0.81 | 2.96E−03 | 2.22E−02 |
| Ubiquitin-conjugating | CDC34 | P49427 | 0.49 | 0.16 | 0.82 | 3.97E−03 | 2.76E−02 |
| enzyme E2 R1 | | | | | | | |
| Ubiquitin-like modifier- | ATG7 | O95352 | 0.49 | 0.18 | 0.79 | 1.83E−03 | 1.55E−02 |
| activating enzyme ATG7 | | | | | | | |
| Keratin, type I cytoskeletal | KRT20 | P35900 | 0.49 | 0.17 | 0.8 | 2.74E−03 | 2.10E−02 |
| 20 | | | | | | | |
| Quinone oxidoreductase | TP5313 | Q53FA7 | 0.49 | 0.17 | 0.8 | 2.29E−03 | 1.85E−02 |
| PIG3 | | | | | | | |
| Beta-crystallin B1 | CRYBB1 | P53674 | 0.49 | 0.14 | 0.85 | 6.77E−03 | 4.21E−02 |
| Focal adhesion kinase 1 | PTK2 | Q05397 | 0.49 | 0.15 | 0.84 | 5.37E−03 | 3.53E−02 |
| Proenkephalin-A | PENK | P01210 | 0.49 | 0.16 | 0.82 | 3.34E−03 | 2.45E−02 |
| Neuropilin and tolloid-like | NETO2 | Q8NC67 | 0.48 | 0.14 | 0.81 | 4.89E−03 | 3.30E−02 |
| protein 2 | | | | | | | |
| Syntaxin-8 | STX8 | Q9UNK0 | 0.48 | 0.22 | 0.75 | 3.82E−04 | 4.55E−03 |
| Rab GDP dissociation | GDI1 | P31150 | 0.48 | 0.18 | 0.78 | 1.64E−03 | 1.41E−02 |
| inhibitor alpha | | | | | | | |
| Polymeric | PIGR | P01833 | 0.48 | 0.2 | 0.76 | 7.55E−04 | 7.65E−03 |
| immunoglobulin receptor | | | | | | | |
| N-terminal pro-BNP | NPPB | P16860 | 0.48 | 0.3 | 0.66 | 1.88E−07 | 5.90E−06 |
| Peroxisomal membrane | PEX14 | O75381 | 0.48 | 0.14 | 0.82 | 6.12E−03 | 3.92E−02 |
| protein PEX14: N-term | | | | | | | |
| BRISC complex subunit | FAM175B | Q15018 | 0.48 | 0.14 | 0.83 | 6.52E−03 | 4.09E−02 |
| Abro1 | | | | | | | |
| Dolichyl- | RPN1 | P04843 | 0.47 | 0.17 | 0.78 | 2.36E−03 | 1.89E−02 |
| diphosphooligosaccharide-- | | | | | | | |
| protein | | | | | | | |
| glycosyltransferase subunit | | | | | | | |
| 1: Lumenal domain | | | | | | | |
| AarF domain-containing | ADCK4 | Q96D53 | 0.47 | 0.13 | 0.8 | 6.47E−03 | 4.06E−02 |
| protein kinase 4 | | | | | | | |
| Collagen alpha-1(X) chain | COL10A1 | Q03692 | 0.46 | 0.13 | 0.8 | 7.20E−03 | 4.40E−02 |
| Protein canopy homolog 3 | CNPY3 | Q9BT09 | 0.46 | 0.2 | 0.71 | 4.15E−04 | 4.85E−03 |
| Probable tRNA(His) | THG1L | Q9NWX6 | 0.46 | 0.13 | 0.78 | 6.28E−03 | 3.99E−02 |
| guanylyltransferase | | | | | | | |
| Protein FAM49B | FAM49B | Q9NUQ9 | 0.46 | 0.13 | 0.8 | 6.73E−03 | 4.19E−02 |
| Complement C3b, | C3 | P01024 | 0.46 | 0.13 | 0.79 | 6.19E−03 | 3.95E−02 |
| inactivated | | | | | | | |
| L-lactate dehydrogenase B | LDHB | P07195 | 0.46 | 0.12 | 0.81 | 7.97E−03 | 4.75E−02 |
| chain | | | | | | | |
| Tapasin | TAPBP | O15533 | 0.45 | 0.17 | 0.73 | 1.45E−03 | 1.28E−02 |
| cAMP-dependent protein | PRKAR1B | P31321 | 0.45 | 0.15 | 0.74 | 3.26E−03 | 2.41E−02 |
| kinase type I-beta | | | | | | | |
| regulatory subunit | | | | | | | |
| Gamma-interferon- | IFI16 | Q16666 | 0.45 | 0.18 | 0.72 | 9.27E−04 | 9.00E−03 |
| inducible protein | | | | | | | |
| 16: Isoform 2, | | | | | | | |
| Hematopoietic expression, | | | | | | | |
| interferon-inducible | | | | | | | |
| nature, and nuclear | | | | | | | |
| localization 1 | | | | | | | |
| Acyl-CoA-binding protein | DBI | P07108 | 0.45 | 0.14 | 0.76 | 4.30E−03 | 2.95E−02 |
| Alpha-ketoglutarate- | ALKBH3 | Q96Q83 | 0.45 | 0.14 | 0.76 | 4.65E−03 | 3.16E−02 |
| dependent dioxygenase | | | | | | | |
| alkB homolog 3 | | | | | | | |
| Low affinity | FCER2 | P06734 | 0.45 | 0.13 | 0.78 | 6.81E−03 | 4.23E−02 |
| immunoglobulin epsilon | | | | | | | |
| Fc receptor | | | | | | | |
| Granulocyte colony- | CSF3 | P09919 | 0.45 | 0.17 | 0.73 | 1.74E−03 | 1.49E−02 |
| stimulating factor | | | | | | | |
| C-C motif chemokine 19 | CCL19 | Q99731 | 0.45 | 0.15 | 0.75 | 3.29E−03 | 2.42E−02 |
| Chitotriosidase-1 | CHIT1 | Q13231 | 0.44 | 0.23 | 0.65 | 3.87E−05 | 6.36E−04 |
| Torsin-1A-interacting | TOR1AIP1 | Q5JTV8 | 0.43 | 0.13 | 0.73 | 4.98E−03 | 3.35E−02 |
| protein 1: Perinuclear | | | | | | | |
| domain | | | | | | | |
| Coiled-coil domain- | CCDC50 | Q8IVM0 | 0.43 | 0.13 | 0.72 | 4.90E−03 | 3.31E−02 |
| containing protein 50 | | | | | | | |
| Ubiquitin carboxyl- | USP14 | P54578 | 0.43 | 0.14 | 0.71 | 3.60E−03 | 2.59E−02 |
| terminal hydrolase 14 | | | | | | | |
| Ubiquitin + 1, truncated | RPS27A | P62979 | 0.43 | 0.14 | 0.72 | 3.60E−03 | 2.59E−02 |
| mutation for UbB | | | | | | | |
| Serine protease inhibitor | SPINK8 | P0C7L1 | 0.43 | 0.12 | 0.73 | 6.44E−03 | 4.06E−02 |
| Kazal-type 8 | | | | | | | |
| 60S ribosomal protein L30 | RPL30 | P62888 | 0.42 | 0.15 | 0.69 | 2.04E−03 | 1.70E−02 |
| Probable RNA-binding | EIF1AD | Q8N9N8 | 0.42 | 0.11 | 0.72 | 7.26E−03 | 4.42E−02 |
| protein EIF1AD | | | | | | | |
| Interferon-induced GTP- | MX1 | P20591 | 0.42 | 0.16 | 0.68 | 1.39E−03 | 1.24E−02 |
| binding protein Mx1 | | | | | | | |
| SHC-transforming protein | SHC1 | P29353 | 0.42 | 0.16 | 0.68 | 1.67E−03 | 1.43E−02 |
| 1: Src Homology domain | | | | | | | |
| Protein WFDC11 | WFDC11 | Q8NEX6 | 0.42 | 0.17 | 0.67 | 9.26E−04 | 9.00E−03 |
| Potassium/sodium | HCN1 | O60741 | 0.41 | 0.12 | 0.71 | 5.67E−03 | 3.69E−02 |
| hyperpolarization- | | | | | | | |
| activated cyclic | | | | | | | |
| nucleotide-gated channel 1 | | | | | | | |
| C-X-C motif chemokine 9 | CXCL9 | Q07325 | 0.41 | 0.12 | 0.7 | 5.21E−03 | 3.46E−02 |
| Integrin alpha-I: beta-1 | ITGA1 | P05556\|P56199 | 0.4 | 0.12 | 0.68 | 5.21E−03 | 3.46E−02 |
| complex | ITGB1 | | | | | | |
| Ubiquilin-4 | UBQLN4 | Q9NRR5 | 0.39 | 0.12 | 0.66 | 4.18E−03 | 2.88E−02 |
| Ras GTPase-activating | G3BP1 | Q13283 | 0.38 | 0.11 | 0.65 | 6.22E−03 | 3.96E−02 |
| protein-binding protein 1 | | | | | | | |
| Platelet-derived growth | PDGFRL | Q15198 | 0.38 | 0.1 | 0.66 | 7.14E−03 | 4.37E−02 |
| factor receptor-like protein | | | | | | | |
| Beta-defensin 4A | DEFB4A | O15263 | 0.37 | 0.23 | 0.51 | 3.14E−07 | 9.34E−06 |
| Cadherin-17 | CDH17 | Q12864 | 0.37 | 0.11 | 0.62 | 5.21E−03 | 3.46E−02 |
| Fc receptor-like B | FCRLB | Q6BAA4 | 0.36 | 0.12 | 0.61 | 3.96E−03 | 2.75E−02 |
| Hepatitis A virus cellular | HAVCR1 | Q96D42 | 0.36 | 0.12 | 0.61 | 3.65E−03 | 2.60E−02 |
| receptor 1 | | | | | | | |
| Bile salt sulfotransferase | SULT2A1 | Q06520 | 0.36 | 0.12 | 0.61 | 3.73E−03 | 2.65E−02 |
| Signal transducer and | STAT1 | P42224 | 0.35 | 0.15 | 0.55 | 5.62E−04 | 6.12E−03 |
| activator of transcription 1- | | | | | | | |
| alpha/beta | | | | | | | |
| eIF-2-alpha kinase GCN2 | EIF2AK4 | Q9P2K8 | 0.35 | 0.11 | 0.59 | 4.78E−03 | 3.24E−02 |
| Receptor-transporting | RTP4 | Q96DX8 | 0.35 | 0.1 | 0.59 | 6.16E−03 | 3.94E−02 |
| protein 4 | | | | | | | |
| Beta-microseminoprotein | MSMB | P08118 | 0.34 | 0.12 | 0.55 | 2.48E−03 | 1.96E−02 |
| Roundabout homolog 4 | ROBO4 | Q8WZ75 | 0.34 | 0.12 | 0.56 | 2.13E−03 | 1.74E−02 |
| Acetyl-CoA | ACAT2 | Q9BWD1 | 0.34 | 0.16 | 0.52 | 2.62E−04 | 3.34E−03 |
| acetyltransferase, cytosolic | | | | | | | |
| Glucagon | GCG | P01275 | 0.34 | 0.09 | 0.58 | 6.71E−03 | 4.18E−02 |
| Odorant-binding protein | OBP2B | Q9NPH6 | 0.34 | 0.1 | 0.59 | 5.64E−03 | 3.68E−02 |
| 2b | | | | | | | |
| Ras GTPase-activating | G3BP2 | Q9UN86 | 0.34 | 0.12 | 0.56 | 2.41E−03 | 1.91E−02 |
| protein-binding protein 2 | | | | | | | |
| Forkhead box protein M1 | FOXM1 | Q08050 | 0.33 | 0.11 | 0.55 | 3.19E−03 | 2.36E−02 |
| Nucleoside diphosphate | NME4 | O00746 | 0.33 | 0.14 | 0.51 | 6.15E−04 | 6.55E−03 |
| kinase, mitochondrial | | | | | | | |
| Interstitial collagenase | MMP1 | P03956 | 0.33 | 0.11 | 0.55 | 3.01E−03 | 2.25E−02 |
| Bcl2-associated agonist of | BAD | Q92934 | 0.33 | 0.09 | 0.57 | 6.30E−03 | 3.99E−02 |
| cell death | | | | | | | |
| SCP2 sterol-binding | SCP2D1 | Q9UJQ7 | 0.32 | 0.1 | 0.53 | 3.74E−03 | 2.65E−02 |
| domain-containing protein | | | | | | | |
| 1 | | | | | | | |
| Inhibin beta C chain | INHBC | P55103 | 0.32 | 0.1 | 0.55 | 5.23E−03 | 3.47E−02 |
| Glutaredoxin-3 | GLRX3 | O76003 | 0.32 | 0.09 | 0.56 | 6.71E−03 | 4.18E−02 |
| Beta-2-microglobulin | B2M | P61769 | 0.3 | 0.12 | 0.49 | 1.49E−03 | 1.31E−02 |
| Neurotensin/neuromedin N | NTS | P30990 | 0.3 | 0.15 | 0.46 | 1.40E−04 | 1.93E−03 |
| Hydroxymethylglutaryl- | HMGCS1 | Q01581 | 0.29 | 0.08 | 0.5 | 6.95E−03 | 4.29E−02 |
| CoA synthase, cytoplasmic | | | | | | | |
| Transcription factor 4 | TCF4 | P15884 | 0.28 | 0.07 | 0.49 | 7.83E−03 | 4.69E−02 |
| Deoxyuridine 5′- | DUT | P33316 | 0.26 | 0.07 | 0.46 | 7.56E−03 | 4.56E−02 |
| triphosphate | | | | | | | |
| nucleotidohydrolase, | | | | | | | |
| mitochondrial | | | | | | | |
| Chitinase-3-like protein 1 | CHI3L1 | P36222 | 0.22 | 0.08 | 0.35 | 1.56E−03 | 1.36E−02 |
| Zymogen granule | ZG16 | O60844 | 0.22 | 0.16 | 0.29 | 1.18E−10 | 7.92E−09 |
| membrane protein 16 | | | | | | | |
| Thioredoxin-like protein | TXNLAB | Q9NX01 | 0.21 | 0.05 | 0.36 | 8.09E−03 | 4.79E−02 |
| 4B | | | | | | | |
| Cytochrome c oxidase | COX7A2L | O14548 | 0.2 | 0.07 | 0.33 | 3.33E−03 | 2.44E−02 |
| subunit 7A-related protein, | | | | | | | |
| mitochondrial | | | | | | | |
| GMP reductase 1 | GMPR | P36959 | 0.17 | 0.08 | 0.26 | 1.20E−04 | 1.68E−03 |
| Immunoglobulin D | IGHD IGK@ | P01880 | 0.14 | 0.04 | 0.25 | 6.31E−03 | 3.99E−02 |
| | IGL@ | | | | | | |
| Tapasin-related protein | TAPBPL | Q9BX59 | −0.18 | −0.3 | −0.05 | 4.60E−03 | 3.13E−02 |
| Hepcidin | HAMP | P81172 | −0.2 | −0.34 | −0.05 | 6.93E−03 | 4.28E−02 |
| Platelet-derived growth | PDGFB | P01127 | −0.24 | −0.41 | −0.07 | 5.82E−03 | 3.76E−02 |
| factor subunit B | | | | | | | |
| Tumor protein p53- | TP53I11 | O14683 | −0.29 | −0.48 | −0.1 | 2.77E−03 | 2.11E−02 |
| inducible protein 11 | | | | | | | |
| Brain-derived neurotrophic | BDNF | P23560 | −0.29 | −0.47 | −0.1 | 2.88E−03 | 2.17E−02 |
| factor | | | | | | | |
| Human Chorionic | CGA CGB | P0DN87\|P0DN86\|P01215 | −0.31 | −0.49 | −0.13 | 8.49E−04 | 8.36E−03 |
| Gonadotropin | | | | | | | |
| Cochlin | COCH | O43405 | −0.31 | −0.53 | −0.08 | 7.70E−03 | 4.62E−02 |
| Fibronectin | FN1 | P02751 | −0.32 | −0.53 | −0.12 | 2.33E−03 | 1.87E−02 |
| Platelet-derived growth | PDGFA | P04085 | −0.32 | −0.55 | −0.09 | 5.68E−03 | 3.69E−02 |
| factor subunit A | | | | | | | |
| Leptin receptor, soluble | LEPR | P48357 | −0.32 | −0.56 | −0.08 | 8.15E−03 | 4.81E−02 |
| Fructose-bisphosphate | ALDOB | P05062 | −0.35 | −0.59 | −0.12 | 2.72E−03 | 2.09E−02 |
| aldolase B | | | | | | | |
| Carboxypeptidase B | CPB1 | P15086 | −0.36 | −0.6 | −0.12 | 3.89E−03 | 2.72E−02 |
| Follicle stimulating | CGA FSHB | P01225\|P01215 | −0.37 | −0.61 | −0.13 | 2.50E−03 | 1.97E−02 |
| hormone | | | | | | | |
| Leukocyte | LILRB1 | Q8NHL6 | −0.37 | −0.56 | −0.18 | 1.59E−04 | 2.16E−03 |
| immunoglobulin-like | | | | | | | |
| receptor subfamily B | | | | | | | |
| member 1 | | | | | | | |
| C-C motif chemokine 5 | CCL5 | P13501 | −0.37 | −0.58 | −0.15 | 8.21E−04 | 8.17E−03 |
| Annexin A6 | ANXA6 | P08133 | −0.38 | −0.63 | −0.14 | 2.41E−03 | 1.91E−02 |
| Fibronectin Fragment 3 | FN1 | P02751 | −0.39 | −0.68 | −0.1 | 8.17E−03 | 4.81E−02 |
| Aldehyde dehydrogenase | ALDH1A3 | P47895 | −0.4 | −0.65 | −0.14 | 2.04E−03 | 1.70E−02 |
| family 1 member A3 | | | | | | | |
| Pancreatic triacylglycerol | PNLIP | P16233 | −0.42 | −0.66 | −0.18 | 5.31E−04 | 5.88E−03 |
| lipase | | | | | | | |
| Oligodendrocyte-myelin | OMG | P23515 | −0.42 | −0.72 | −0.12 | 5.50E−03 | 3.61E−02 |
| glycoprotein | | | | | | | |
| Apolipoprotein E | APOE | P02649 | −0.42 | −0.68 | −0.16 | 1.81E−03 | 1.54E−02 |
| Cathelicidin antimicrobial | CAMP | P49913 | −0.42 | −0.72 | −0.13 | 4.84E−03 | 3.28E−02 |
| peptide | | | | | | | |
| Lactotransferrin | LTF | P02788 | −0.43 | −0.73 | −0.13 | 4.65E−03 | 3.16E−02 |
| Protein Wnt-5a | WNT5A | P41221 | −0.44 | −0.76 | −0.11 | 7.92E−03 | 4.73E−02 |
| Chymotrypsinogen B | CTRB1 | P17538 | −0.45 | −0.72 | −0.19 | 7.91E−04 | 7.92E−03 |
| Probable | DIMT1 | Q9UNQ2 | −0.46 | −0.78 | −0.15 | 3.70E−03 | 2.63E−02 |
| dimethyladenosine | | | | | | | |
| transferase | | | | | | | |
| Inactive gamma- | GGT2 | P36268 | −0.46 | −0.78 | −0.14 | 5.10E−03 | 3.41E−02 |
| glutamyltranspeptidase 2 | | | | | | | |
| Apolipoprotein C-III | APOC3 | P02656 | −0.47 | −0.67 | −0.26 | 7.24E−06 | 1.52E−04 |
| Insulin-like growth factor- | IGFBP5 | P24593 | −0.48 | −0.84 | −0.13 | 7.38E−03 | 4.48E−02 |
| binding protein 5 | | | | | | | |
| Ficolin-2 | FCN2 | Q15485 | −0.5 | −0.79 | −0.22 | 5.77E−04 | 6.23E−03 |
| C-type lectin domain | CLEC4C | Q8WTT0 | −0.51 | −0.84 | −0.18 | 2.17E−03 | 1.77E−02 |
| family 4 member C | | | | | | | |
| Chondrocalcin | COL2A1 | P02458 | −0.52 | −0.76 | −0.29 | 1.18E−05 | 2.33E−04 |
| Signal peptide, CUB and | SCUBE1 | Q8IWY4 | −0.52 | −0.76 | −0.28 | 2.10E−05 | 3.77E−04 |
| EGF-like domain- | | | | | | | |
| containing protein 1 | | | | | | | |
| Insulin-like growth factor I | IGF1 | P05019 | −0.54 | −0.94 | −0.14 | 7.69E−03 | 4.62E−02 |
| Platelet basic protein | PPBP | P02775 | −0.55 | −0.9 | −0.19 | 2.40E−03 | 1.91E−02 |
| Fatty acid-binding protein | FABP9 | Q0Z7S8 | −0.55 | −0.95 | −0.15 | 6.45E−03 | 4.06E−02 |
| 9 | | | | | | | |
| Trypsin-3 | PRSS3 | P35030 | −0.55 | −0.85 | −0.26 | 2.70E−04 | 3.41E−03 |
| C-C motif chemokine 1 | CCL1 | P22362 | −0.55 | −0.97 | −0.14 | 8.49E−03 | 4.97E−02 |
| Advanced glycosylation | AGER | Q15109 | −0.55 | −0.77 | −0.34 | 2.88E−07 | 8.74E−06 |
| end product-specific | | | | | | | |
| receptor, soluble | | | | | | | |
| Normal mucosa of | NMES1 | Q9C002 | −0.55 | −0.9 | −0.2 | 2.34E−03 | 1.87E−02 |
| esophagus-specific gene 1 | | | | | | | |
| protein | | | | | | | |
| Protein kinase C-binding | NELL1 | Q92832 | −0.55 | −0.88 | −0.22 | 1.17E−03 | 1.08E−02 |
| protein NELL1 | | | | | | | |
| Solute carrier family 35 | SLC35G2 | Q8TBE7 | −0.56 | −0.89 | −0.22 | 1.05E−03 | 1.00E−02 |
| member G2 | | | | | | | |
| Melanocyte protein PMEL | PMEL | P40967 | −0.56 | −0.93 | −0.18 | 3.62E−03 | 2.59E−02 |
| Synaptic vesicle | VAT1 | Q99536 | −0.57 | −0.91 | −0.23 | 9.84E−04 | 9.46E−03 |
| membrane protein VAT-1 | | | | | | | |
| homolog | | | | | | | |
| Kininostatin | KNG1 | P01042 | −0.57 | −0.84 | −0.29 | 4.94E−05 | 7.76E−04 |
| Cyclin-dependent kinase | CDK5 | Q15078\|Q00535 | −0.57 | −0.95 | −0.2 | 2.78E−03 | 2.12E−02 |
| 5: Cyclin-dependent kinase | CDK5R1 | | | | | | |
| 5 activator 1 complex | | | | | | | |
| Membrane protein | FAM174A | Q8TBP5 | −0.57 | −0.95 | −0.19 | 3.36E−03 | 2.45E−02 |
| FAM174A | | | | | | | |
| Cartilage intermediate | CILP2 | Q8IUL8 | −0.57 | −0.98 | −0.17 | 5.51E−03 | 3.61E−02 |
| layer protein 2 | | | | | | | |
| Beta-Ala-His dipeptidase | CNDP1 | Q96KN2 | −0.58 | −0.8 | −0.37 | 1.40E−07 | 4.70E−06 |
| Vesicular integral- | LMAN2 | Q12907 | −0.58 | −1.01 | −0.15 | 8.42E−03 | 4.94E−02 |
| membrane protein VIP36 | | | | | | | |
| Fibronectin Fragment 4 | FN1 | P02751 | −0.59 | −0.98 | −0.19 | 3.64E−03 | 2.60E−02 |
| Glypican-3 | GPC3 | P51654 | −0.59 | −0.98 | −0.21 | 2.66E−03 | 2.06E−02 |
| NADH-cytochrome b5 | CYB5R3 | P00387 | −0.59 | −0.89 | −0.3 | 9.66E−05 | 1.40E−03 |
| reductase 3 | | | | | | | |
| Kv channel-interacting | KCNIP1 | Q9NZI2 | −0.6 | −1.04 | −0.16 | 7.11E−03 | 4.37E−02 |
| protein 1 | | | | | | | |
| Protein CEI | C5orf38 | Q86SI9 | −0.6 | −0.96 | −0.24 | 1.06E−03 | 1.01E−02 |
| Sodium channel subunit | SCN2B | O60939 | −0.62 | −1.06 | −0.19 | 5.32E−03 | 3.51E−02 |
| beta-2 | | | | | | | |
| Carbonic anhydrase- | CA10 | Q9NS85 | −0.63 | −1.09 | −0.17 | 7.05E−03 | 4.33E−02 |
| related protein 10 | | | | | | | |
| Coagulation Factor V | F5 | P12259 | −0.63 | −1.06 | −0.19 | 4.46E−03 | 3.06E−02 |
| Glypican-5 | GPC5 | P78333 | −0.63 | −1 | −0.25 | 1.02E−03 | 9.74E−03 |
| SLIT and NTRK-like | SLITRK3 | O94933 | −0.64 | −0.9 | −0.38 | 1.87E−06 | 4.67E−05 |
| protein 3 | | | | | | | |
| BPI fold-containing family | BPIFA2 | Q96DR5 | −0.64 | −1.05 | −0.24 | 1.74E−03 | 1.49E−02 |
| A member 2 | | | | | | | |
| Beta-defensin 1 | DEFB1 | P60022 | −0.64 | −1.06 | −0.21 | 3.13E−03 | 2.33E−02 |
| Cerebellin-1 | CBLN1 | P23435 | −0.64 | −0.94 | −0.35 | 2.35E−05 | 4.14E−04 |
| Hematopoietic | HPGDS | O60760 | −0.65 | −1.08 | −0.22 | 2.83E−03 | 2.14E−02 |
| prostaglandin D synthase | | | | | | | |
| Inositol monophosphatase | IMPAD1 | Q9NX62 | −0.65 | −1.13 | −0.17 | 8.49E−03 | 4.97E−02 |
| 3 | | | | | | | |
| Desmoglein-1 | DSG1 | Q02413 | −0.66 | −1.13 | −0.18 | 6.98E−03 | 4.30E−02 |
| von Willebrand factor A | VWA2 | Q5GFL6 | −0.66 | −1.1 | −0.22 | 3.49E−03 | 2.53E−02 |
| domain-containing protein | | | | | | | |
| 2 | | | | | | | |
| Carbonic anhydrase 6 | CA6 | P23280 | −0.67 | −0.89 | −0.44 | 1.24E−08 | 5.19E−07 |
| Platelet-derived growth | PDGFRB | P09619 | −0.67 | −0.97 | −0.36 | 1.70E−05 | 3.14E−04 |
| factor receptor beta | | | | | | | |
| Interleukin-34 | IL34 | Q6ZMJ4 | −0.67 | −1.09 | −0.26 | 1.45E−03 | 1.28E−02 |
| DCN1-like protein 5 | DCUN1D5 | Q9BTE7 | −0.67 | −1.04 | −0.3 | 3.64E−04 | 4.37E−03 |
| Polypeptide N- | GALNT13 | Q8IUC8 | −0.68 | −1.09 | −0.27 | 1.23E−03 | 1.12E−02 |
| acetylgalactosaminyltransferase | | | | | | | |
| 13 | | | | | | | |
| Apolipoprotein A-IV | APOA4 | P06727 | −0.68 | −1.15 | −0.2 | 5.13E−03 | 3.43E−02 |
| Complement component | C8A C8B | P07360\|P07358\|P07357 | −0.68 | −1.1 | −0.27 | 1.23E−03 | 1.12E−02 |
| C8 | C8G | | | | | | |
| Ectonucleotide | ENPP5 | Q9UJA9 | −0.68 | −1.13 | −0.24 | 2.73E−03 | 2.09E−02 |
| pyrophosphatase/phosphod | | | | | | | |
| iesterase family member 5 | | | | | | | |
| Apolipoprotein A-V | APOA5 | Q6Q788 | −0.69 | −0.98 | −0.41 | 1.84E−06 | 4.63E−05 |
| Cysteine-rich protein 1 | CRIP1 | P50238 | −0.69 | −1.08 | −0.3 | 5.28E−04 | 5.88E−03 |
| CD209 antigen | CD209 | Q9NNX6 | −0.69 | −1.04 | −0.34 | 1.17E−04 | 1.65E−03 |
| Heparan sulfate | HS3ST5 | Q8IZT8 | −0.7 | −1.15 | −0.26 | 2.08E−03 | 1.72E−02 |
| glucosamine 3-O- | | | | | | | |
| sulfotransferase 5 | | | | | | | |
| Proactivator polypeptide- | PSAPL1 | Q6NUJ1 | −0.7 | −1.11 | −0.3 | 6.96E−04 | 7.17E−03 |
| like 1 | | | | | | | |
| Ficolin-2 | FCN2 | Q15485 | −0.71 | −1.12 | −0.29 | 7.83E−04 | 7.88E−03 |
| Proprotein convertase | PCSK7 | Q16549 | −0.71 | −1.23 | −0.19 | 7.44E−03 | 4.51E−02 |
| subtilisin/kexin type 7 | | | | | | | |
| Protein Wnt-3a | WNT3A | P56704 | −0.72 | −1.13 | −0.32 | 4.63E−04 | 5.30E−03 |
| Serine/threonine-protein | STK16 | O75716 | −0.72 | −1.24 | −0.2 | 6.83E−03 | 4.24E−02 |
| kinase 16 | | | | | | | |
| Arfaptin-2 | ARFIP2 | P53365 | −0.73 | −1.09 | −0.36 | 9.33E−05 | 1.36E−03 |
| Angiogenic factor with G | AGGF1 | Q8N302 | −0.73 | −1.21 | −0.25 | 2.83E−03 | 2.14E−02 |
| patch and FHA domains 1 | | | | | | | |
| Protocadherin beta-2 | PCDHB2 | Q9Y5E7 | −0.74 | −1.21 | −0.28 | 1.66E−03 | 1.43E−02 |
| Fibroblast growth factor | FGFR3 | P22607 | −0.74 | −1.16 | −0.32 | 5.88E−04 | 6.32E−03 |
| receptor 3: Extracellular | | | | | | | |
| domain | | | | | | | |
| Coagulation factor XIII | F13A1 F13B | P05160\|P00488 | −0.74 | −1.24 | −0.24 | 3.91E−03 | 2.73E−02 |
| Protein MENT | MENT | Q9BUN1 | −0.74 | −1.14 | −0.33 | 3.62E−04 | 4.37E−03 |
| Growth hormone receptor | GHR | P10912 | −0.75 | −1.19 | −0.31 | 8.63E−04 | 8.45E−03 |
| Mono [ADP-ribose] | PARP16 | Q8N5Y8 | −0.75 | −1.23 | −0.27 | 2.06E−03 | 1.70E−02 |
| polymerase PARP16 | | | | | | | |
| Translocon-associated | SSR1 | P43307 | −0.75 | −1.3 | −0.19 | 8.16E−03 | 4.81E−02 |
| protein subunit alpha | | | | | | | |
| Inositol polyphosphate 5- | OCRL | Q01968 | −0.76 | −1.31 | −0.2 | 8.00E−03 | 4.76E−02 |
| phosphatase OCRL-1 | | | | | | | |
| Ras-related protein Rab-17 | RAB17 | Q9H0T7 | −0.77 | −1.29 | −0.26 | 3.39E−03 | 2.47E−02 |
| Properdin | CFP | P27918 | −0.77 | −1.3 | −0.25 | 3.78E−03 | 2.66E−02 |
| Dual specificity mitogen- | MAP2K4 | P45985 | −0.77 | −1.13 | −0.41 | 3.27E−05 | 5.51E−04 |
| activated protein kinase | | | | | | | |
| kinase 4 | | | | | | | |
| Selenoprotein S | VIMP | Q9BQE4 | −0.78 | −1.14 | −0.41 | 2.70E−05 | 4.67E−04 |
| NT-3 growth factor | NTRK3 | Q16288 | −0.78 | −1.35 | −0.22 | 6.70E−03 | 4.18E−02 |
| receptor | | | | | | | |
| Type 2 lactosamine alpha- | ST3GAL6 | Q9Y274 | −0.78 | −1.23 | −0.34 | 5.40E−04 | 5.97E−03 |
| 2,3-sialyltransferase | | | | | | | |
| N-acetylglucosamine-1- | NAGPA | Q9UK23 | −0.79 | −1.3 | −0.27 | 2.76E−03 | 2.11E−02 |
| phosphodiester alpha-N- | | | | | | | |
| acetylglucosaminidase | | | | | | | |
| Antibacterial protein LL- | CAMP | P49913 | −0.79 | −1.22 | −0.36 | 2.91E−04 | 3.65E−03 |
| 37 | | | | | | | |
| Semaphorin-4B | SEMA4B | Q9NPR2 | −0.79 | −1.33 | −0.25 | 4.05E−03 | 2.80E−02 |
| Regulator of G-protein | RGS8 | P57771 | −0.8 | −1.37 | −0.23 | 6.37E−03 | 4.02E−02 |
| signaling 8 | | | | | | | |
| Uncharacterized protein | C14orf93 | Q9H972 | −0.8 | −1.37 | −0.24 | 5.10E−03 | 3.41E−02 |
| C14orf93 | | | | | | | |
| Leucine-rich repeat | LRRK2 | Q5S007 | −0.81 | −1.29 | −0.33 | 1.05E−03 | 9.99E−03 |
| serine/threonine-protein | | | | | | | |
| kinase 2 | | | | | | | |
| Proteasome subunit alpha | PSMA5 | P28066 | −0.81 | −1.27 | −0.36 | 4.94E−04 | 5.59E−03 |
| type-5 | | | | | | | |
| Semenogelin-1 | SEMG1 | P04279 | −0.81 | −1.4 | −0.22 | 7.45E−03 | 4.51E−02 |
| Coxsackievirus and | CXADR | P78310 | −0.82 | −1.35 | −0.28 | 2.65E−03 | 2.06E−02 |
| adenovirus receptor | | | | | | | |
| Guanylate-binding protein | GBP6 | Q6ZN66 | −0.82 | −1.35 | −0.29 | 2.51E−03 | 1.98E−02 |
| 6 | | | | | | | |
| Carboxypeptidase M | CPM | P14384 | −0.82 | −1.41 | −0.23 | 6.11E−03 | 3.91E−02 |
| Beta-1,4- | B4GALT6 | Q9UBX8 | −0.83 | −1.35 | −0.31 | 1.83E−03 | 1.55E−02 |
| galactosyltransferase 6 | | | | | | | |
| Prostaglandin F2 receptor | PTGFRN | Q9P2B2 | −0.83 | −1.32 | −0.34 | 8.50E−04 | 8.36E−03 |
| negative regulator | | | | | | | |
| Glutathione S-transferase | GSTA4 | O15217 | −0.83 | −1.34 | −0.32 | 1.50E−03 | 1.32E−02 |
| A4 | | | | | | | |
| Histone-lysine N- | EHMT2 | Q96KQ7 | −0.83 | −1.28 | −0.37 | 3.50E−04 | 4.24E−03 |
| methyltransferase EHMT2 | | | | | | | |
| Leukocyte | LILRA4 | P59901 | −0.83 | −1.3 | −0.37 | 4.09E−04 | 4.80E−03 |
| immunoglobulin-like | | | | | | | |
| receptor subfamily A | | | | | | | |
| member 4 | | | | | | | |
| Heme oxygenase 1 | HMOX1 | P09601 | −0.84 | −1.31 | −0.38 | 4.03E−04 | 4.77E−03 |
| Mast/stem cell growth | KIT | P10721 | −0.84 | −1.29 | −0.38 | 3.15E−04 | 3.91E−03 |
| factor receptor Kit | | | | | | | |
| Cadherin-7 | CDH7 | Q9ULB5 | −0.84 | −1.3 | −0.38 | 3.45E−04 | 4.20E−03 |
| T-cell surface glycoprotein | CD3E | P07766 | −0.84 | −1.39 | −0.29 | 2.67E−03 | 2.07E−02 |
| CD3 epsilon chain | | | | | | | |
| Macrophage-stimulating | MST1R | Q04912 | −0.85 | −1.39 | −0.31 | 2.12E−03 | 1.74E−02 |
| protein receptor | | | | | | | |
| Coagulation factor Xa | F10 | P00742 | −0.85 | −1.42 | −0.28 | 3.62E−03 | 2.59E−02 |
| Secretogranin-3 | SCG3 | Q8WXD2 | −0.85 | −1.42 | −0.28 | 3.58E−03 | 2.57E−02 |
| Retinol-binding protein 4 | RBP4 | P02753 | −0.86 | −1.35 | −0.37 | 5.49E−04 | 6.02E−03 |
| Ribosomal protein S6 | RPS6KB1 | P23443 | −0.86 | −1.38 | −0.35 | 1.09E−03 | 1.03E−02 |
| kinase beta-1 | | | | | | | |
| Potassium voltage-gated | KCNF1 | Q9H3M0 | −0.87 | −1.5 | −0.24 | 6.69E−03 | 4.18E−02 |
| channel subfamily F | | | | | | | |
| member 1 | | | | | | | |
| Hexokinase-1 | HK1 | P19367 | −0.87 | −1.46 | −0.29 | 3.57E−03 | 2.57E−02 |
| Histone acetyltransferase | HAT1 | O14929 | −0.87 | −1.25 | −0.5 | 5.35E−06 | 1.18E−04 |
| type B catalytic subunit | | | | | | | |
| Aurora kinase B | AURKB | Q96GD4 | −0.88 | −1.52 | −0.25 | 6.37E−03 | 4.02E−02 |
| Iron-sulfur cluster co- | HSCB | Q8IWL3 | −0.89 | −1.47 | −0.31 | 2.77E−03 | 2.11E−02 |
| chaperone protein HscB, | | | | | | | |
| mitochondrial | | | | | | | |
| Protein SERAC1 | SERAC1 | Q96JX3 | −0.89 | −1.47 | −0.31 | 2.65E−03 | 2.06E−02 |
| Plasma protease C1 | SERPING1 | P05155 | −0.91 | −1.43 | −0.39 | 6.35E−04 | 6.71E−03 |
| inhibitor | | | | | | | |
| Kidney-associated antigen | KAAG1 | Q9UBP8 | −0.93 | −1.52 | −0.35 | 1.63E−03 | 1.41E−02 |
| 1 | | | | | | | |
| Urotensin-2 | UTS2 | O95399 | −0.93 | −1.49 | −0.37 | 1.21E−03 | 1.11E−02 |
| Voltage-dependent | CACNA2D3 | Q8IZS8 | −0.93 | −1.43 | −0.42 | 3.12E−04 | 3.88E−03 |
| calcium channel subunit | | | | | | | |
| alpha-2/delta-3 | | | | | | | |
| Liver carboxylesterase 1 | CES1 | P23141 | −0.94 | −1.47 | −0.42 | 4.11E−04 | 4.82E−03 |
| Surfeit locus protein 1 | SURF1 | Q15526 | −0.94 | −1.33 | −0.55 | 2.35E−06 | 5.82E−05 |
| Sodium-coupled | SLC5A8 | Q8N695 | −0.95 | −1.4 | −0.5 | 3.31E−05 | 5.55E−04 |
| monocarboxylate | | | | | | | |
| transporter 1 | | | | | | | |
| Endothelial monocyte- | AIMP1 | Q12904 | −0.95 | −1.63 | −0.28 | 5.49E−03 | 3.60E−02 |
| activating polypeptide 2 | | | | | | | |
| V-set and transmembrane | VSTM4 | Q8IW00 | −0.95 | −1.65 | −0.25 | 8.08E−03 | 4.79E−02 |
| domain-containing protein | | | | | | | |
| 4 | | | | | | | |
| Protein shisa-2 homolog | SHISA2 | Q6UWI4 | −0.96 | −1.56 | −0.36 | 1.83E−03 | 1.55E−02 |
| Coagulation Factor X | F10 | P00742 | −0.96 | −1.54 | −0.38 | 1.19E−03 | 1.09E−02 |
| Coactosin-like protein | COTL1 | Q14019 | −0.96 | −1.61 | −0.3 | 4.13E−03 | 2.85E−02 |
| Carboxypeptidase N | CPN2 | P22792 | −0.96 | −1.59 | −0.32 | 3.23E−03 | 2.39E−02 |
| subunit 2 | | | | | | | |
| Appetite-regulating | GHRL | Q9UBU3 | −0.96 | −1.55 | −0.37 | 1.32E−03 | 1.19E−02 |
| hormone | | | | | | | |
| Potassium voltage-gated | KCNG4 | Q8TDN1 | −0.97 | −1.64 | −0.3 | 4.48E−03 | 3.06E−02 |
| channel subfamily G | | | | | | | |
| member 4 | | | | | | | |
| tRNA (guanine-N(7)-)- | METTL1 | Q9UBP6 | −0.98 | −1.7 | −0.27 | 7.00E−03 | 4.31E−02 |
| methyltransferase | | | | | | | |
| Interleukin-1 Receptor | IL1RAP | Q9NPH3 | −0.98 | −1.38 | −0.58 | 1.53E−06 | 3.91E−05 |
| accessory protein | | | | | | | |
| Interleukin-1 receptor type | IL1R2 | P27930 | −0.98 | −1.56 | −0.41 | 7.41E−04 | 7.54E−03 |
| 2 | | | | | | | |
| Apolipoprotein B | APOB | P04114 | −0.98 | −1.43 | −0.52 | 2.86E−05 | 4.90E−04 |
| Platelet-activating factor | PLA2G7 | Q13093 | −0.98 | −1.4 | −0.56 | 4.48E−06 | 1.01E−04 |
| acetylhydrolase | | | | | | | |
| Chymotrypsin-like elastase | CELA2A | P08217 | −0.98 | −1.31 | −0.65 | 5.55E−09 | 2.43E−07 |
| family member 2A | | | | | | | |
| Serine/threonine-protein | PAK3 | O75914 | −0.99 | −1.49 | −0.48 | 1.30E−04 | 1.80E−03 |
| kinase PAK 3 | | | | | | | |
| Serine/threonine-protein | PIM1 | P11309 | −0.99 | −1.59 | −0.39 | 1.18E−03 | 1.09E−02 |
| kinase pim-1 | | | | | | | |
| Sperm-associated antigen | SPAG11A | Q6PDA7 | −0.99 | −1.51 | −0.47 | 2.12E−04 | 2.77E−03 |
| 11A | | | | | | | |
| Golgi-associated plant | GLIPR2 | Q9H4G4 | −1 | −1.6 | −0.4 | 1.05E−03 | 1.00E−02 |
| pathogenesis-related | | | | | | | |
| protein 1 | | | | | | | |
| 1-phosphatidylinositol 4,5- | PLCG1 | P19174 | −1 | −1.67 | −0.32 | 3.81E−03 | 2.67E−02 |
| bisphosphate phosphodiesterase gamma- | | | | | | | |
| 1 | | | | | | | |
| Cytoskeleton-associated | CKAP2 | Q8WWK9 | −1 | −1.56 | −0.43 | 5.48E−04 | 6.02E−03 |
| protein 2 | | | | | | | |
| Oxysterols receptor LXR- | NR1H2 | P55055 | −1 | −1.71 | −0.29 | 5.96E−03 | 3.83E−02 |
| beta | | | | | | | |
| Phosphatidylinositol | PITPNB | P48739 | −1.01 | −1.7 | −0.33 | 3.75E−03 | 2.65E−02 |
| transfer protein beta | | | | | | | |
| isoform | | | | | | | |
| Galanin | GAL | P22466 | −1.01 | −1.76 | −0.27 | 7.53E−03 | 4.55E−02 |
| BDNF/NT-3 growth | NTRK2 | Q16620 | −1.01 | −1.5 | −0.53 | 4.52E−05 | 7.19E−04 |
| factors receptor | IGLON5 | A6NGN9 | −1.01 | −1.54 | −0.48 | 1.91E−04 | |
| IgLON family member 5 | | | | | | | 2.53E−03 |
| Amyloid-like protein 1 | APLP1 | P51693 | −1.01 | −1.37 | −0.65 | 4.82E−08 | 1.79E−06 |
| G2/mitotic-specific cyclin- | CCNB2 | O95067 | −1.02 | −1.72 | −0.33 | 3.76E−03 | 2.65E−02 |
| B2 | | | | | | | |
| Endothelin-converting | ECE1 | P42892 | −1.02 | −1.65 | −0.39 | 1.57E−03 | 1.36E−02 |
| enzyme 1: Cytoplasmic | | | | | | | |
| domain | | | | | | | |
| Thiopurine S- | TPMT | P51580 | −1.03 | −1.62 | −0.44 | 6.10E−04 | 6.51E−03 |
| methyltransferase | | | | | | | |
| Adrenomedullin | ADM | P35318 | −1.04 | −1.81 | −0.28 | 7.60E−03 | 4.57E−02 |
| Fibrinogen beta chain | FGB | P02675 | −1.04 | −1.69 | −0.39 | 1.77E−03 | 1.51E−02 |
| Aggrecan core protein | ACAN | P16112 | −1.04 | −1.57 | −0.5 | 1.38E−04 | 1.91E−03 |
| Ectonucleoside | ENTPD5 | O75356 | −1.04 | −1.66 | −0.43 | 9.16E−04 | 8.95E−03 |
| triphosphate | | | | | | | |
| diphosphohydrolase 5 | | | | | | | |
| Chromobox protein | CBX5 | P45973 | −1.04 | −1.61 | −0.46 | 4.23E−04 | 4.91E−03 |
| homolog 5 | | | | | | | |
| C-type lectin domain | CLEC4D | Q8WXI8 | −1.04 | −1.74 | −0.34 | 3.45E−03 | 2.51E−02 |
| family 4 member D | | | | | | | |
| Cell adhesion molecule- | CDON | Q4KMG0 | −1.05 | −1.69 | −0.4 | 1.43E−03 | 1.27E−02 |
| related/down-regulated by | | | | | | | |
| oncogenes | | | | | | | |
| M-phase inducer | CDC25B | P30305 | −1.06 | −1.81 | −0.32 | 4.98E−03 | 3.35E−02 |
| phosphatase 2 | | | | | | | |
| NADH dehydrogenase | NDUFB11 | Q9NX14 | −1.06 | −1.72 | −0.39 | 1.85E−03 | 1.56E−02 |
| [ubiquinone] 1 beta | | | | | | | |
| subcomplex subunit 11, | | | | | | | |
| mitochondrial | | | | | | | |
| Ficolin-3 | FCN3 | O75636 | −1.07 | −1.54 | −0.6 | 7.79E−06 | 1.62E−04 |
| Putative hydrolase RBBP9 | RBBP9 | O75884 | −1.08 | −1.85 | −0.32 | 5.23E−03 | 3.47E−02 |
| Kininogen, HMW, Two | KNG1 | P01042 | −1.08 | −1.49 | −0.68 | 1.45E−07 | 4.79E−06 |
| Chain | | | | | | | |
| Peptidase inhibitor 15 | PI15 | O43692 | −1.08 | −1.87 | −0.29 | 7.30E−03 | 4.44E−02 |
| Prenylcysteine oxidase 1 | PCYOX1 | Q9UHG3 | −1.08 | −1.56 | −0.6 | 9.68E−06 | 1.95E−04 |
| Dixin | DIXDC1 | Q155Q3 | −1.09 | −1.86 | −0.32 | 5.70E−03 | 3.70E−02 |
| Syndecan-3 | SDC3 | O75056 | −1.09 | −1.61 | −0.56 | 5.08E−05 | 7.88E−04 |
| Receptor-type tyrosine- | PTPRD | P23468 | −1.09 | −1.58 | −0.61 | 1.16E−05 | 2.31E−04 |
| protein phosphatase delta | | | | | | | |
| Coagulation factor VII | F7 | P08709 | −1.1 | −1.58 | −0.62 | 8.16E−06 | 1.66E−04 |
| Ubiquitin D | UBD | O15205 | −1.11 | −1.83 | −0.38 | 2.71E−03 | 2.09E−02 |
| 26S proteasome non- | PSMD10 | O75832 | −1.12 | −1.88 | −0.35 | 4.27E−03 | 2.94E−02 |
| ATPase regulatory subunit | | | | | | | |
| 10 | | | | | | | |
| Leucine-rich repeat and | LRFN1 | Q9P244 | −1.12 | −1.78 | −0.47 | 7.07E−04 | 7.22E−03 |
| fibronectin type III | | | | | | | |
| domain-containing protein | | | | | | | |
| 1 | | | | | | | |
| Coagulation Factor XI | F11 | P03951 | −1.13 | −1.75 | −0.51 | 3.69E−04 | 4.42E−03 |
| Anthrax toxin receptor 1 | ANTXR1 | Q9H6X2 | −1.14 | −1.68 | −0.6 | 4.00E−05 | 6.53E−04 |
| Small conductance | KCNN1 | Q92952 | −1.14 | −1.89 | −0.4 | 2.73E−03 | 2.09E−02 |
| calcium-activated | | | | | | | |
| potassium channel protein | | | | | | | |
| 1 | | | | | | | |
| Insulin-like growth factor- | IGFBP3 | P17936 | −1.15 | −1.64 | −0.66 | 4.40E−06 | 9.96E−05 |
| binding protein 3 | | | | | | | |
| Transmembrane protein | TMEM9 | Q9P0T7 | −1.15 | −1.86 | −0.44 | 1.44E−03 | 1.27E−02 |
| 9: Cytoplasmic domain | | | | | | | |
| Potassium voltage-gated | KCNE5 | Q9UJ90 | −1.15 | −1.81 | −0.49 | 6.58E−04 | 6.89E−03 |
| channel subfamily E | | | | | | | |
| regulatory beta subunit | | | | | | | |
| 5: Cytoplasmic domain | | | | | | | |
| rRNA 2′-O- | FBL | P22087 | −1.16 | −1.85 | −0.46 | 1.17E−03 | 1.09E−02 |
| methyltransferase | | | | | | | |
| fibrillarin | | | | | | | |
| Vitamin K-dependent | PROS1 | P07225 | −1.16 | −1.91 | −0.41 | 2.37E−03 | 1.89E−02 |
| protein S | | | | | | | |
| Calcium-activated | KCNMB3 | Q9NPA1 | −1.16 | −1.82 | −0.49 | 6.58E−04 | 6.89E−03 |
| potassium channel subunit | | | | | | | |
| beta-3 | | | | | | | |
| Neurocan core protein | NCAN | O14594 | −1.2 | −1.6 | −0.8 | 5.06E−09 | 2.25E−07 |
| Stromelysin-1 | MMP3 | P08254 | −1.2 | −1.73 | −0.67 | 9.86E−06 | 1.98E−04 |
| Coagulation factor XIII B | F13B | P05160 | −1.2 | −1.75 | −0.64 | 2.29E−05 | 4.06E−04 |
| chain | | | | | | | |
| EKC/KEOPS complex | TPRKB | Q9Y3C4 | −1.21 | −1.93 | −0.49 | 1.06E−03 | 1.01E−02 |
| subunit TPRKB | | | | | | | |
| Myosin regulatory light | MYL7 | Q01449 | −1.21 | −1.73 | −0.69 | 5.26E−06 | 1.17E−04 |
| chain 2, atrial isoform | | | | | | | |
| Retinoblastoma-associated | RB1 | P06400 | −1.21 | −1.85 | −0.57 | 2.12E−04 | 2.77E−03 |
| protein | | | | | | | |
| Anthrax toxin receptor 2 | ANTXR2 | P58335 | −1.22 | −1.68 | −0.76 | 2.07E−07 | 6.41E−06 |
| Cytochrome c oxidase | COX4I2 | Q96KJ9 | −1.22 | −1.82 | −0.62 | 7.29E−05 | 1.08E−03 |
| subunit 4 isoform 2, | | | | | | | |
| mitochondrial | | | | | | | |
| Sialic acid-binding Ig-like | SIGLEC12 | Q96PQ1 | −1.22 | −1.8 | −0.63 | 5.04E−05 | 7.87E−04 |
| lectin 12: Ig-like C2-type 2 | | | | | | | |
| domain, Isoform short | | | | | | | |
| Zinc finger protein 175 | ZNF175 | Q9Y473 | −1.23 | −2.04 | −0.41 | 3.16E−03 | 2.35E−02 |
| Tartrate-resistant acid | ACP5 | P13686 | −1.24 | −1.86 | −0.61 | 1.16E−04 | 1.64E−03 |
| phosphatase type 5 | | | | | | | |
| Desmoglein-2 | DSG2 | Q14126 | −1.26 | −1.83 | −0.7 | 1.30E−05 | 2.55E−04 |
| Immunoglobulin | IGDCC4 | Q8TDY8 | −1.26 | −1.85 | −0.67 | 2.77E−05 | 4.77E−04 |
| superfamily DCC subclass | | | | | | | |
| member 4 | | | | | | | |
| Repulsive guidance | RGMA | Q96B86 | −1.29 | −2.16 | −0.42 | 3.76E−03 | 2.65E−02 |
| molecule A | | | | | | | |
| E3 ubiquitin-protein ligase | CHFR | Q96EP1 | −1.31 | −2.15 | −0.48 | 2.09E−03 | 1.72E−02 |
| CHFR | | | | | | | |
| Glucoside | GXYLT1 | Q4G148 | −1.31 | −1.86 | −0.75 | 4.20E−06 | 9.64E−05 |
| xylosyltransferase 1 | | | | | | | |
| Contactin-4 | CNTN4 | Q8IWV2 | −1.34 | −2.07 | −0.62 | 2.99E−04 | 3.73E−03 |
| Insulin-like growth factor- | IGFALS | P35858 | −1.34 | −1.77 | −0.91 | 1.34E−09 | 7.12E−08 |
| binding protein complex | | | | | | | |
| acid labile subunit | | | | | | | |
| Solute carrier family 22 | SLC22A16 | Q86VW1 | −1.35 | −1.86 | −0.85 | 1.61E−07 | 5.17E−06 |
| member 16 | | | | | | | |
| Cartilage acidic protein 1 | CRTAC1 | Q9NQ79 | −1.36 | −1.87 | −0.85 | 1.58E−07 | 5.17E−06 |
| Platelet endothelial | PEAR1 | Q5VY43 | −1.36 | −2.15 | −0.58 | 6.60E−04 | 6.89E−03 |
| aggregation receptor | | | | | | | |
| 1: Extracellular domain | | | | | | | |
| Neuronal growth regulator | NEGR1 | Q7Z3B1 | −1.37 | −2.22 | −0.51 | 1.84E−03 | 1.55E−02 |
| 1 | | | | | | | |
| Semaphorin-3G | SEMA3G | Q9NS98 | −1.38 | −2.06 | −0.7 | 7.22E−05 | 1.07E−03 |
| Leucine-rich repeats and | LRIG3 | Q6UXM1 | −1.39 | −1.96 | −0.83 | 1.17E−06 | 3.03E−05 |
| immunoglobulin-like | | | | | | | |
| domains protein 3 | | | | | | | |
| Thrombin | F2 | P00734 | −1.39 | −1.75 | −1.03 | 5.14E−14 | 6.92E−12 |
| Tumor necrosis factor | TNFSF12 | O43508 | −1.4 | −1.98 | −0.81 | 3.36E−06 | 7.98E−05 |
| ligand superfamily | | | | | | | |
| member 12 | | | | | | | |
| Palmitoleoyl-protein | NOTUM | Q6P988 | −1.4 | −1.79 | −1.02 | 1.15E−12 | 1.25E−10 |
| carboxylesterase NOTUM | | | | | | | |
| Torsin-1 A-interacting | TOR1AIP1 | Q5JTV8 | −1.4 | −1.92 | −0.88 | 1.43E−07 | 4.79E−06 |
| protein 1: Nuclear domain | | | | | | | |
| ADP-ribosylation factor | ARFGAP1 | Q8N6T3 | −1.42 | −2.15 | −0.69 | 1.47E−04 | 2.02E−03 |
| GTPase-activating protein | | | | | | | |
| 1 | | | | | | | |
| Interleukin-6 receptor | IL6R | P08887 | −1.42 | −1.92 | −0.91 | 4.27E−08 | 1.61E−06 |
| subunit alpha | | | | | | | |
| Neuronal pentraxin-2 | NPTX2 | P47972 | −1.42 | −2.09 | −0.74 | 4.26E−05 | 6.89E−04 |
| Carbonic anhydrase 4 | CA4 | P22748 | −1.43 | −2.13 | −0.73 | 6.57E−05 | 9.88E−04 |
| Baculoviral LAP repeat- | BIRC5 | O15392 | −1.5 | −2.3 | −0.69 | 2.73E−04 | 3.44E−03 |
| containing protein 5 | | | | | | | |
| Hedgehog-interacting | HHIP | Q96QV1 | −1.51 | −2.19 | −0.83 | 1.38E−05 | 2.68E−04 |
| protein | | | | | | | |
| SH2 domain-containing | SH2D3C | Q8N5H7 | −1.52 | −2.24 | −0.8 | 3.52E−05 | 5.84E−04 |
| protein 3C | | | | | | | |
| Uncharacterized protein | C3orf18 | Q9UK00 | −1.52 | −2.35 | −0.69 | 3.40E−04 | 4.18E−03 |
| C3orf18 | | | | | | | |
| Zona pellucida-binding | ZPBP | Q9BS86 | −1.55 | −2.58 | −0.51 | 3.49E−03 | 2.53E−02 |
| protein 1 | | | | | | | |
| Heat shock 70 kDa protein | HSPA1A | PODMV8 | −1.56 | −2.27 | −0.85 | 1.60E−05 | 3.00E−04 |
| 1A | | | | | | | |
| E3 ubiquitin-protein ligase | RNF146 | Q9NTX7 | −1.57 | −2.32 | −0.81 | 5.08E−05 | 7.88E−04 |
| RNF146 | | | | | | | |
| Zinc finger protein 334 | ZNF334 | Q9HCZ1 | −1.57 | −2.33 | −0.81 | 4.85E−05 | 7.66E−04 |
| A disintegrin and | ADAMTS13 | Q76LX8 | −1.58 | −2.08 | −1.09 | 4.38E−10 | 2.60E−08 |
| metalloproteinase with | | | | | | | |
| thrombospondin motifs 13 | | | | | | | |
| F-box/LRR-repeat protein | FBXLA | Q9UKA2 | −1.6 | −2.52 | −0.69 | 5.67E−04 | 6.15E−03 |
| 4: Leucine-rich repeats 4 | | | | | | | |
| and 5 | | | | | | | |
| Vascular endothelial | KDR | P35968 | −1.6 | −2.32 | −0.88 | 1.35E−05 | 2.64E−04 |
| growth factor receptor 2 | | | | | | | |
| Inter-alpha-trypsin | ITIH1 | P19827 | −1.62 | −2.36 | −0.89 | 1.57E−05 | 2.98E−04 |
| inhibitor heavy chain H1 | | | | | | | |
| Sodium/potassium- | ATP1B1 | P05026 | −1.63 | −2.02 | −1.23 | 8.54E−16 | 1.64E−13 |
| transporting ATPase | | | | | | | |
| subunit beta-1 | | | | | | | |
| Cholinesterase | BCHE | P06276 | −1.63 | −2.21 | −1.04 | 5.43E−08 | 1.98E−06 |
| Alpha-2-HS-glycoprotein | AHSG | P02765 | −1.64 | −2.47 | −0.81 | 1.06E−04 | 1.52E−03 |
| Calsequestrin-1 | CASQ1 | P31415 | −1.65 | −2.58 | −0.72 | 4.79E−04 | 5.45E−03 |
| Biotinidase | BTD | P43251 | −1.65 | −2.36 | −0.95 | 4.20E−06 | 9.64E−05 |
| Growth/differentiation | GDF2 | Q9UK05 | −1.65 | −2.37 | −0.93 | 7.33E−06 | 1.53E−04 |
| factor 2 | | | | | | | |
| TOM1-like protein 1 | TOM1L1 | O75674 | −1.67 | −2.34 | −1.01 | 9.27E−07 | 2.44E−05 |
| Hepatocyte growth factor | MET | P08581 | −1.68 | −2.36 | −1.01 | 9.32E−07 | 2.44E−05 |
| receptor | | | | | | | |
| Complement component 1 | C1QBP | Q07021 | −1.7 | −2.6 | −0.79 | 2.48E−04 | 3.18E−03 |
| Q subcomponent-binding | | | | | | | |
| protein, mitochondrial | | | | | | | |
| Carboxypeptidase B2 | CPB2 | Q96IY4 | −1.71 | −2.66 | −0.76 | 4.07E−04 | 4.79E−03 |
| E3 ubiquitin-protein ligase | DTX1 | Q86Y01 | −1.74 | −2.49 | −0.99 | 5.92E−06 | 1.29E−04 |
| DTX1 | | | | | | | |
| Complement component | C8G | P07360 | −1.74 | −2.52 | −0.95 | 1.47E−05 | 2.81E−04 |
| C8 gamma chain | | | | | | | |
| Mannosyl-oligosaccharide | MAN1A2 | O60476 | −1.79 | −2.81 | −0.77 | 5.64E−04 | 6.14E−03 |
| 1,2-alpha-mannosidase IB | | | | | | | |
| Vitamin D-binding protein | GC | P02774 | −1.8 | −2.76 | −0.83 | 2.65E−04 | 3.36E−03 |
| Zinc-alpha-2-glycoprotein | AZGP1 | P25311 | −1.82 | −2.45 | −1.19 | 1.56E−08 | 6.30E−07 |
| Collagen alpha-1(XIII) | COL13A1 | Q5TAT6 | −1.89 | −2.52 | −1.26 | 4.93E−09 | 2.21E−07 |
| chain | | | | | | | |
| N-acetylmuramoyl-L- | PGLYRP2 | Q96PD5 | −1.92 | −2.72 | −1.11 | 3.06E−06 | 7.35E−05 |
| alanine amidase | | | | | | | |
| Muellerian-inhibiting | AMH | P03971 | −1.97 | −2.65 | −1.3 | 1.01E−08 | 4.27E−07 |
| factor | | | | | | | |
| S-methyl-5′-thioadenosine | MTAP | Q13126 | −2.03 | −2.79 | −1.27 | 1.64E−07 | 5.24E−06 |
| phosphorylase | | | | | | | |
| Superoxide dismutase | SOD2 | P04179 | −2.04 | −2.58 | −1.51 | 5.94E−14 | 7.78E−12 |
| [Mn], mitochondrial | | | | | | | |
| Afamin | AFM | P43652 | −2.06 | −2.78 | −1.34 | 2.42E−08 | 9.49E−07 |
| Complement C1q tumor | C1QTNF3 | Q9BXJ4 | −2.06 | −2.7 | −1.42 | 2.82E−10 | 1.69E−08 |
| necrosis factor-related | | | | | | | |
| protein 3 | | | | | | | |
| Protein SET | SET | Q01105 | −2.08 | −2.95 | −1.22 | 2.37E−06 | 5.82E−05 |
| Neuronal growth regulator | NEGR1 | Q7Z3B1 | −2.09 | −2.97 | −1.21 | 3.28E−06 | 7.82E−05 |
| 1 | | | | | | | |
| Cadherin-3 | CDH3 | P22223 | −2.09 | −2.68 | −1.49 | 9.35E−12 | 7.89E−10 |
| Receptor-type tyrosine- | PTPRJ | Q12913 | −2.1 | −2.8 | −1.4 | 4.70E−09 | 2.15E−07 |
| protein phosphatase eta | | | | | | | |
| Antithrombin-III | SERPINC1 | P01008 | −2.17 | −3.6 | −0.74 | 3.01E−03 | 2.25E−02 |
| Plasma kallikrein | KLKB1 | P03952 | −2.24 | −3.02 | −1.46 | 2.12E−08 | 8.44E−07 |
| EF-hand domain- | EFHD1 | Q9BUP0 | −2.27 | −3.21 | −1.33 | 2.37E−06 | 5.82E−05 |
| containing protein D1 | | | | | | | |
| Alpha-2-antiplasmin | SERPINF2 | P08697 | −2.28 | −3.41 | −1.15 | 7.73E−05 | 1.14E−03 |
| Coiled-coil domain- | CCDC126 | Q96EE4 | −2.29 | −2.83 | −1.75 | 1.50E−16 | 3.55E−14 |
| containing protein 126 | | | | | | | |
| Ciliary neurotrophic factor | CNTFR | P26992 | −2.32 | −3 | −1.64 | 3.26E−11 | 2.39E−09 |
| receptor subunit alpha | | | | | | | |
| Group XIIB secretory | PLA2G12B | Q9BX93 | −2.33 | −2.82 | −1.84 | 2.80E−20 | 1.16E−17 |
| phospholipase A2-like | | | | | | | |
| protein | | | | | | | |
| Contactin-1 | CNTN1 | Q12860 | −2.36 | −3.11 | −1.61 | 6.82E−10 | 3.95E−08 |
| Matrix-remodeling- | MXRA8 | Q9BRK3 | −2.39 | −3.04 | −1.74 | 6.88E−13 | 7.97E−11 |
| associated protein | | | | | | | |
| 8: Extracellular domain | | | | | | | |
| Heparan-sulfate 6-O- | HS6ST3 | Q8IZP7 | −2.46 | −3.39 | −1.54 | 1.87E−07 | 5.90E−06 |
| sulfotransferase 3 | | | | | | | |
| Kallistatin | SERPINA4 | P29622 | −2.51 | −3.48 | −1.54 | 3.93E−07 | 1.14E−05 |
| Neural cell adhesion | NCAM1 | P13591 | −2.58 | −3.28 | −1.88 | 8.10E−13 | 9.17E−11 |
| molecule 1, 120 kDa | | | | | | | |
| isoform | | | | | | | |
| Receptor-type tyrosine- | PTPRS | Q13332 | −2.6 | −3.45 | −1.76 | 1.69E−09 | 8.59E−08 |
| protein phosphatase S | | | | | | | |
| Inter-alpha-trypsin | ITIH2 | P19823 | −2.72 | −3.67 | −1.78 | 1.49E−08 | 6.13E−07 |
| inhibitor heavy chain H2 | | | | | | | |
| B melanoma antigen 3 | BAGE3 | Q86Y29 | −2.76 | −3.56 | −1.96 | 1.77E−11 | 1.47E−09 |
| Vitamin K-dependent | PROC | P04070 | −2.79 | −3.51 | −2.07 | 3.80E−14 | 5.26E−12 |
| protein C | | | | | | | |
| Bifunctional heparan | NDST1 | P52848 | −2.83 | −3.86 | −1.8 | 6.95E−08 | 2.42E−06 |
| sulfate N-deacetylase/N- | | | | | | | |
| sulfotransferase 1 | | | | | | | |
| Serum albumin | ALB | P02768 | −2.84 | −3.93 | −1.75 | 3.36E−07 | 9.89E−06 |
| Apolipoprotein M | APOM | O95445 | −2.85 | −3.43 | −2.28 | 2.63E−22 | 1.87E−19 |
| Tetranectin | CLEC3B | P05452 | −2.91 | −3.8 | −2.01 | 2.33E−10 | 1.43E−08 |
| Neuropeptide S | NPS | P0C0P6 | −3.28 | −4.12 | −2.44 | 2.91E−14 | 4.39E−12 |
| Apolipoprotein C-I | APOC1 | P02654 | −3.4 | −4.35 | −2.44 | 3.69E−12 | 3.40E−10 |
| Neurogenic locus notch | NOTCH1 | P46531 | −3.42 | −4.67 | −2.17 | 9.48E−08 | 3.23E−06 |
| homolog protein 1 | | | | | | | |
| Epidermal growth factor | EGFR | P00533 | −4.35 | −5.26 | −3.44 | 1.04E−20 | 4.73E−18 |
| receptor | | | | | | | |

In the CARDIA cohort, 185 proteins (183 GeneIDs) were significantly associated with QIA, of which 136 were positively and 49 negatively associated (Table 5).

TABLE 5
Multivariable regression analysis of QIA in CARDIA

| | Entrez | UniProt | Percent QIA | Upper | Lower | | FDR |
|---|---|---|---|---|---|---|---|
| Protein name | gene name | ID | per ln(RFU) | 95% CI | 95% CI | p-value | p-value |
| Insulin-like growth factor-binding | IGFBP4 | P22692 | 2.66 | 1.74 | 3.58 | 1.72E−08 | 1.71E−05 |
| protein 4 | | | | | | | |
| C-X-C motif chemokine 16 | CXCL16 | Q9H2A7 | 2.58 | 1.62 | 3.54 | 1.62E−07 | 1.04E−04 |
| Complement C1q tumor necrosis factor- | C1QTNF1 | Q9BXJ1 | 2.57 | 1.43 | 3.71 | 1.04E−05 | 1.67E−03 |
| related protein 1 | | | | | | | |
| Lumican | LUM | P51884 | 2.56 | 1.55 | 3.58 | 7.65E−07 | 2.70E−04 |
| Tumor necrosis factor ligand | TNFSF15 | O95150 | 2.54 | 1.58 | 3.49 | 2.23E−07 | 1.11E−04 |
| superfamily member 15 | | | | | | | |
| Neurofascin | NFASC | O94856 | 2.45 | 1.39 | 3.51 | 5.90E−06 | 1.22E−03 |
| Neuropilin-2 | NRP2 | O60462 | 2.37 | 1.09 | 3.64 | 2.73E−04 | 1.32E−02 |
| Complement factor B | CFB | P00751 | 2.32 | 0.87 | 3.77 | 1.73E−03 | 4.77E−02 |
| Fibulin-5 | FBLN5 | Q9UBX5 | 2.29 | 1.2 | 3.39 | 3.97E−05 | 3.66E−03 |
| Zinc finger protein 75D | ZNF75D | P51815 | 2.2 | 1.48 | 2.91 | 1.78E−09 | 4.44E−06 |
| Matrix Gla protein | MGP | P08493 | 2.08 | 1.05 | 3.11 | 7.77E−05 | 5.78E−03 |
| Antileukoproteinase | SLPI | P03973 | 2.07 | 0.99 | 3.15 | 1.70E−04 | 9.49E−03 |
| Embryonal Fyn-associated substrate | EFS | O43281 | 2.02 | 0.98 | 3.06 | 1.37E−04 | 8.43E−03 |
| Rho GTPase-activating protein 36 | ARHGAP36 | Q6ZRI8 | 2 | 1.16 | 2.85 | 3.27E−06 | 7.74E−04 |
| Complement C2 | C2 | P06681 | 1.98 | 1 | 2.96 | 7.35E−05 | 5.66E−03 |
| WAP four-disulfide core domain protein | WFDC2 | Q14508 | 1.96 | 1.26 | 2.67 | 5.01E−08 | 4.16E−05 |
| 2 | | | | | | | |
| Nidogen-1 | NID1 | P14543 | 1.88 | 1 | 2.76 | 2.88E−05 | 3.19E−03 |
| Collagen alpha-1(XXVIII) chain | COL28A1 | Q2UY09 | 1.85 | 0.98 | 2.72 | 3.03E−05 | 3.21E−03 |
| Interleukin-20 receptor subunit alpha | IL20RA | Q9UHF4 | 1.85 | 0.95 | 2.75 | 5.67E−05 | 4.70E−03 |
| B melanoma antigen 2 | BAGE2 | Q86Y30 | 1.82 | 1.04 | 2.61 | 5.51E−06 | 1.19E−03 |
| Leucine-rich repeats and | LRIG1 | Q96JA1 | 1.8 | 0.94 | 2.65 | 3.89E−05 | 3.65E−03 |
| immunoglobulin-like domains protein 1 | | | | | | | |
| Low-density lipoprotein receptor-related | LRP11 | Q86VZ4 | 1.79 | 0.9 | 2.67 | 7.50E−05 | 5.66E−03 |
| protein 11 | | | | | | | |
| EGF-containing fibulin-like extracellular | EFEMP1 | Q12805 | 1.79 | 0.82 | 2.76 | 2.96E−04 | 1.39E−02 |
| matrix protein 1 | | | | | | | |
| Collagen alpha-3(VI) chain: Bovine | COL6A3 | P12111 | 1.78 | 0.92 | 2.63 | 4.49E−05 | 3.99E−03 |
| pancreatic trypsin inhibitor/Kunitz | | | | | | | |
| inhibitor domain, isoform 1 | | | | | | | |
| Beta-2-microglobulin | B2M | P61769 | 1.78 | 1.11 | 2.45 | 1.96E−07 | 1.09E−04 |
| Sushi, von Willebrand factor type A, | SVEP1 | Q4LDE5 | 1.73 | 1.07 | 2.39 | 3.23E−07 | 1.46E−04 |
| EGF and pentraxin domain-containing | | | | | | | |
| protein 1: EGF-like domains 4-6 | | | | | | | |
| Cystatin-C | CST3 | P01034 | 1.71 | 0.82 | 2.61 | 1.74E−04 | 9.51E−03 |
| RNA-binding protein EWS | EWSR1 | Q01844 | 1.7 | 0.76 | 2.64 | 3.97E−04 | 1.70E−02 |
| C-C motif chemokine 21 | CCL21 | O00585 | 1.67 | 1 | 2.33 | 8.13E−07 | 2.70E−04 |
| Cell growth regulator with EF hand | CGREF1 | Q99674 | 1.67 | 0.68 | 2.65 | 9.25E−04 | 3.11E−02 |
| domain protein 1 | | | | | | | |
| Serine/arginine-rich splicing factor 6 | SRSF6 | Q13247 | 1.66 | 0.94 | 2.37 | 6.53E−06 | 1.25E−03 |
| Macrophage-capping protein | CAPG | P40121 | 1.66 | 1.11 | 2.21 | 3.06E−09 | 5.08E−06 |
| Interleukin enhancer-binding factor 3 | ILF3 | Q12906 | 1.65 | 0.8 | 2.49 | 1.42E−04 | 8.62E−03 |
| CUB domain-containing protein 1 | CDCP1 | Q9H5V8 | 1.64 | 1.17 | 2.11 | 7.87E−12 | 3.92E−08 |
| Serpin B8 | SERPINB8 | P50452 | 1.64 | 0.81 | 2.46 | 1.05E−04 | 7.28E−03 |
| Spondin-1 | SPON1 | Q9HCB6 | 1.6 | 0.64 | 2.57 | 1.16E−03 | 3.52E−02 |
| Vesicular integral-membrane protein | LMAN2 | Q12907 | 1.6 | 0.67 | 2.53 | 7.59E−04 | 2.70E−02 |
| VIP36 | | | | | | | |
| Ganglioside GM2 activator | GM2A | P17900 | 1.59 | 0.78 | 2.4 | 1.13E−04 | 7.47E−03 |
| Peptidyl-prolyl cis-trans isomerase C | PPIC | P45877 | 1.59 | 0.72 | 2.47 | 3.44E−04 | 1.56E−02 |
| Out at first protein homolog | OAF | Q86UD1 | 1.59 | 0.61 | 2.57 | 1.41E−03 | 4.15E−02 |
| Guanine nucleotide-binding protein | GNGT2 | O14610 | 1.53 | 0.82 | 2.23 | 2.36E−05 | 2.78E−03 |
| G(I)/G(S)/G(O) subunit gamma-T2 | | | | | | | |
| Leukocyte immunoglobulin-like receptor | LILRA5 | A6NI73 | 1.53 | 0.74 | 2.31 | 1.49E−04 | 8.95E−03 |
| subfamily A member 5 | | | | | | | |
| Stem cell growth factor-beta | CLEC11A | Q9Y240 | 1.52 | 0.89 | 2.15 | 2.40E−06 | 5.98E−04 |
| Sushi, von Willebrand factor type A, | SVEP1 | Q4LDE5 | 1.51 | 0.78 | 2.24 | 5.03E−05 | 4.40E−03 |
| EGF and pentraxin domain-containing | | | | | | | |
| protein 1: Sushi 15-18 | | | | | | | |
| Plexin-D1 | PLXND1 | Q9Y4D7 | 1.51 | 0.62 | 2.4 | 8.78E−04 | 2.99E−02 |
| Lamina-associated polypeptide 2, | TMPO | P42167 | 1.49 | 0.88 | 2.11 | 2.28E−06 | 5.98E−04 |
| isoforms beta/gamma | | | | | | | |
| Stem cell growth factor-alpha | CLEC11A | Q9Y240 | 1.47 | 0.89 | 2.05 | 7.13E−07 | 2.70E−04 |
| Protein ABHD14A | ABHD14A | Q9BUJ0 | 1.47 | 0.77 | 2.16 | 3.65E−05 | 3.65E−03 |
| Testis-expressed sequence 29 protein | TEX29 | Q8N6K0 | 1.46 | 0.59 | 2.34 | 1.04E−03 | 3.27E−02 |
| Calsyntenin-3 | CLSTN3 | Q9BQT9 | 1.45 | 0.87 | 2.03 | 9.78E−07 | 3.04E−04 |
| Gamma-glutamyl hydrolase | GGH | Q92820 | 1.44 | 0.68 | 2.21 | 2.30E−04 | 1.17E−02 |
| Neuroblastoma suppressor of | NBL1 | P41271 | 1.42 | 0.71 | 2.12 | 7.95E−05 | 5.82E−03 |
| tumorigenicity 1 | | | | | | | |
| C-X-C motif chemokine 13 | CXCL13 | O43927 | 1.42 | 0.81 | 2.04 | 6.21E−06 | 1.24E−03 |
| Retinoic acid receptor responder protein | RARRES2 | Q99969 | 1.39 | 0.56 | 2.21 | 1.04E−03 | 3.27E−02 |
| 2 | | | | | | | |
| Triggering receptor expressed on | TREM1 | Q9NP99 | 1.39 | 0.78 | 2 | 9.23E−06 | 1.64E−03 |
| myeloid cells 1 | | | | | | | |
| Thioredoxin domain-containing protein | TXNDC5 | Q8NBS9 | 1.38 | 0.56 | 2.2 | 9.97E−04 | 3.22E−02 |
| 5 | | | | | | | |
| Dermatopontin | DPT | Q07507 | 1.37 | 0.52 | 2.21 | 1.54E−03 | 4.41E−02 |
| DnaJ homolog subfamily B member 9 | DNAJB9 | Q9UBS3 | 1.35 | 0.62 | 2.08 | 2.95E−04 | 1.39E−02 |
| ADAMTS-like protein 2 | ADAMTSL2 | Q86TH1 | 1.34 | 0.57 | 2.11 | 6.19E−04 | 2.36E−02 |
| Ephrin type-A receptor 2 | EPHA2 | P29317 | 1.33 | 0.55 | 2.11 | 8.03E−04 | 2.81E−02 |
| Ribonuclease K6 | RNASE6 | Q93091 | 1.33 | 0.68 | 1.98 | 6.26E−05 | 5.11E−03 |
| Oncoprotein-induced transcript 3 protein | OIT3 | Q8WWZ8 | 1.31 | 0.61 | 2.01 | 2.47E−04 | 1.23E−02 |
| Thrombospondin-3 | THBS3 | P49746 | 1.29 | 0.62 | 1.97 | 1.68E−04 | 9.49E−03 |
| Alpha-1-antichymotrypsin complex | SERPINA3 | P01011 | 1.28 | 0.58 | 1.99 | 3.49E−04 | 1.56E−02 |
| UPF0577 protein | ELAPOR1 | Q6UXG2 | 1.28 | 0.54 | 2.03 | 7.14E−04 | 2.56E−02 |
| KIAA1324: Cytoplasmic domain | | | | | | | |
| Serum amyloid A-4 protein | SAA4 | P35542 | 1.27 | 0.74 | 1.79 | 2.22E−06 | 5.98E−04 |
| Growth/differentiation factor 9 | GDF9 | O60383 | 1.26 | 0.59 | 1.92 | 2.05E−04 | 1.08E−02 |
| Trafficking protein particle complex | TRAPPC3 | O43617 | 1.25 | 0.53 | 1.97 | 6.36E−04 | 2.36E−02 |
| subunit 3 | | | | | | | |
| Marginal zone B- and B1-cell-specific | MZB1 | Q8WU39 | 1.25 | 0.73 | 1.78 | 3.45E−06 | 7.81E−04 |
| protein | | | | | | | |
| Tumor necrosis factor receptor | TNFRSF1B | P20333 | 1.25 | 0.6 | 1.9 | 1.69E−04 | 9.49E−03 |
| superfamily member 1B | | | | | | | |
| Transmembrane emp24 domain- | TMED10 | P49755 | 1.21 | 0.52 | 1.9 | 5.80E−04 | 2.26E−02 |
| containing protein 10 | | | | | | | |
| Tumor necrosis factor receptor | TNFRSF17 | Q02223 | 1.19 | 0.48 | 1.91 | 1.10E−03 | 3.43E−02 |
| superfamily member 17 | | | | | | | |
| C-C motif chemokine 22 | CCL22 | O00626 | 1.19 | 0.57 | 1.82 | 1.86E−04 | 9.93E−03 |
| Parathyroid hormone-related protein | PTHLH | P12272 | 1.18 | 0.48 | 1.89 | 9.71E−04 | 3.20E−02 |
| Protein CYR61 | CCN1 | O00622 | 1.18 | 0.57 | 1.79 | 1.56E−04 | 9.24E−03 |
| Secretin | SCT | P09683 | 1.16 | 0.46 | 1.86 | 1.13E−03 | 3.44E−02 |
| Serine/threonine-protein phosphatase 4 | PPP4R3A | Q6IN85 | 1.15 | 0.51 | 1.78 | 4.28E−04 | 1.79E−02 |
| regulatory subunit 3A | | | | | | | |
| Keratin, type I cytoskeletal 18 | KRT18 | P05783 | 1.15 | 0.47 | 1.84 | 1.01E−03 | 3.23E−02 |
| Macrophage metalloelastase | MMP12 | P39900 | 1.14 | 0.64 | 1.63 | 6.89E−06 | 1.27E−03 |
| WAP four-disulfide core domain protein | WFDC1 | Q9HC57 | 1.14 | 0.45 | 1.84 | 1.29E−03 | 3.86E−02 |
| 1 | | | | | | | |
| Intercellular adhesion molecule 1 | ICAM1 | P05362 | 1.13 | 0.6 | 1.66 | 3.00E−05 | 3.21E−03 |
| Troponin T, cardiac muscle | TNNT2 | P45379 | 1.13 | 0.62 | 1.64 | 1.27E−05 | 1.80E−03 |
| Kallikrein-10 | KLK10 | O43240 | 1.12 | 0.5 | 1.75 | 4.24E−04 | 1.79E−02 |
| DOMON domain-containing protein | FRRS1L | Q9POK9 | 1.11 | 0.6 | 1.62 | 2.40E−05 | 2.78E−03 |
| FRRS1L | | | | | | | |
| DnaJ homolog subfamily B member 12 | DNAJB12 | Q9NXW2 | 1.09 | 0.41 | 1.76 | 1.61E−03 | 4.51E−02 |
| C-X-C motif chemokine 10 | CXCL10 | P02778 | 1.08 | 0.59 | 1.56 | 1.25E−05 | 1.80E−03 |
| IGF-like family receptor 1 | IGFLR1 | Q9H665 | 1.08 | 0.57 | 1.6 | 3.82E−05 | 3.65E−03 |
| Interleukin-8 | CXCL8 | P10145 | 1.05 | 0.49 | 1.61 | 2.25E−04 | 1.15E−02 |
| Golgi membrane protein 1 | GOLM1 | Q8NBJ4 | 1.04 | 0.47 | 1.6 | 3.01E−04 | 1.40E−02 |
| E-selectin | SELE | P16581 | 1.02 | 0.51 | 1.53 | 8.31E−05 | 6.00E−03 |
| Lysozyme C | LYZ | P61626 | 1.01 | 0.41 | 1.6 | 9.57E−04 | 3.18E−02 |
| Elafin | PI3 | P19957 | 1.01 | 0.52 | 1.49 | 5.23E−05 | 4.47E−03 |
| Somatoliberin | GHRH | P01286 | 1 | 0.43 | 1.58 | 6.42E−04 | 2.36E−02 |
| Lipopolysaccharide-binding protein | LBP | P18428 | 1 | 0.48 | 1.53 | 1.81E−04 | 9.77E−03 |
| C-C motif chemokine 18 | CCL18 | P55774 | 0.99 | 0.38 | 1.61 | 1.61E−03 | 4.51E−02 |
| Erythropoietin | EPO | P01588 | 0.99 | 0.54 | 1.44 | 1.44E−05 | 1.94E−03 |
| Thioredoxin reductase 1, cytoplasmic | TXNRD1 | Q16881 | 0.96 | 0.41 | 1.51 | 6.25E−04 | 2.36E−02 |
| Syntaxin-12 | STX12 | Q86Y82 | 0.95 | 0.41 | 1.5 | 6.32E−04 | 2.36E−02 |
| Interleukin-3 receptor subunit alpha | IL3RA | P26951 | 0.95 | 0.36 | 1.54 | 1.69E−03 | 4.70E−02 |
| Vascular endothelial growth factor D | VEGFD | O43915 | 0.93 | 0.47 | 1.38 | 6.47E−05 | 5.19E−03 |
| Lactoperoxidase | LPO | P2207 | 0.93 | 0.52 | 1.35 | 1.04E−05 | 1.67E−03 |
| 4F2 cell-surface antigen heavy | SLC3A2 | P08195 | 0.92 | 0.37 | 1.47 | 9.91E−04 | 3.22E−02 |
| chain: Cytoplasmic domain | | | | | | | |
| Thrombospondin-2 | THBS2 | P35442 | 0.91 | 0.42 | 1.41 | 3.05E−04 | 1.41E−02 |
| Axin-2 | AXIN2 | Q9Y2T1 | 0.91 | 0.48 | 1.33 | 2.84E−05 | 3.19E−03 |
| Interferon-induced GTP-binding protein | MX1 | P20591 | 0.9 | 0.44 | 1.35 | 1.09E−04 | 7.33E−03 |
| Mx1 | | | | | | | |
| C-C motif chemokine 19 | CCL19 | Q99731 | 0.9 | 0.41 | 1.39 | 3.52E−04 | 1.56E−02 |
| Phospholipase A2, membrane associated | PLA2G2A | P14555 | 0.88 | 0.37 | 1.39 | 7.04E−04 | 2.54E−02 |
| Gamma-interferon-inducible protein | IFI16 | Q16666 | 0.87 | 0.35 | 1.39 | 1.11E−03 | 3.43E−02 |
| 16: Isoform 2, Hematopoietic expression, | | | | | | | |
| interferon-inducible nature, and nuclear | | | | | | | |
| localization 1 | | | | | | | |
| C-C motif chemokine 3 | CCL3 | P10147 | 0.87 | 0.35 | 1.39 | 9.84E−04 | 3.22E−02 |
| Ubiquitin-like protein ISG15 | ISG15 | P05161 | 0.84 | 0.45 | 1.23 | 2.25E−05 | 2.78E−03 |
| Fibroblast growth factor 23 | FGF23 | Q9GZV9 | 0.84 | 0.4 | 1.27 | 1.58E−04 | 9.24E−03 |
| Growth/differentiation factor 15 | GDF15 | Q99988 | 0.84 | 0.33 | 1.35 | 1.26E−03 | 3.80E−02 |
| Granulocyte colony-stimulating factor | CSF3 | P09919 | 0.82 | 0.4 | 1.25 | 1.24E−04 | 8.03E−03 |
| Interferon regulatory factor 6 | IRF6 | O14896 | 0.82 | 0.4 | 1.23 | 1.34E−04 | 8.36E−03 |
| Carbonic anhydrase 3 | CA3 | P07451 | 0.79 | 0.35 | 1.23 | 4.50E−04 | 1.84E−02 |
| Ribonuclease pancreatic | RNASE1 | P07998 | 0.79 | 0.37 | 1.21 | 2.10E−04 | 1.10E−02 |
| Solute carrier organic anion transporter | SLCO5A1 | Q9H2Y9 | 0.76 | 0.28 | 1.24 | 1.82E−03 | 4.90E−02 |
| family member 5A1 | | | | | | | |
| Cadherin-11: Extracellular domain | CDH11 | P55287 | 0.75 | 0.47 | 1.02 | 1.66E−07 | 1.04E−04 |
| Thiosulfate sulfurtransferase | TST | Q16762 | 0.74 | 0.33 | 1.16 | 4.44E−04 | 1.83E−02 |
| SLAM family member 7 | SLAMF7 | Q9NQ25 | 0.73 | 0.35 | 1.1 | 1.60E−04 | 9.29E−03 |
| Gastrokine-2 | GKN2 | Q86XP6 | 0.72 | 0.31 | 1.13 | 5.00E−04 | 2.02E−02 |
| BPI fold-containing family B member 1 | BPIFB1 | Q8TDL5 | 0.68 | 0.3 | 1.06 | 4.12E−04 | 1.75E−02 |
| Alkaline phosphatase, placental type | ALPP | P05187 | 0.68 | 0.35 | 1.01 | 5.30E−05 | 4.47E−03 |
| ADM2 | ADM2 | Q7Z4H4 | 0.68 | 0.26 | 1.09 | 1.42E−03 | 4.15E−02 |
| Serum amyloid A-2 protein | SAA2 | P0DJI9 | 0.67 | 0.31 | 1.03 | 2.43E−04 | 1.22E−02 |
| Serine protease inhibitor Kazal-type 2 | SPINK2 | P20155 | 0.65 | 0.25 | 1.05 | 1.53E−03 | 4.41E−02 |
| Chitotriosidase-1 | CHIT1 | Q13231 | 0.62 | 0.27 | 0.97 | 5.40E−04 | 2.15E−02 |
| Tapasin | TAPBP | O15533 | 0.61 | 0.27 | 0.95 | 3.78E−04 | 1.65E−02 |
| Growth-regulated alpha protein | CXCL1 | P09341 | 0.6 | 0.25 | 0.96 | 9.11E−04 | 3.09E−02 |
| Troponin I, fast skeletal muscle | TNNI2 | P48788 | 0.6 | 0.26 | 0.94 | 5.72E−04 | 2.25E−02 |
| Beta-defensin 4A | DEFB4A | O15263 | 0.55 | 0.33 | 0.77 | 7.20E−07 | 2.70E−04 |
| C-X-C motif chemokine 11 | CXCL11 | O14625 | 0.47 | 0.18 | 0.75 | 1.35E−03 | 4.02E−02 |
| C-reactive protein | CRP | P02741 | 0.45 | 0.2 | 0.69 | 3.65E−04 | 1.61E−02 |
| Serum amyloid A-1 protein | SAA1 | P0DJI8 | 0.41 | 0.15 | 0.66 | 1.60E−03 | 4.51E−02 |
| Chitinase-3-like protein 1 | CHI3L1 | P36222 | 0.35 | 0.14 | 0.57 | 1.45E−03 | 4.23E−02 |
| Zymogen granule membrane protein 16 | ZG16 | O60844 | 0.21 | 0.14 | 0.29 | 1.26E−08 | 1.56E−05 |
| Teratocarcinoma-derived growth factor 1 | TDGF1 | P13385 | −0.38 | −0.61 | −0.15 | 1.12E−03 | 3.44E−02 |
| Lactase-phlorizin hydrolase | LCT | P09848 | −0.45 | −0.71 | −0.19 | 6.00E−04 | 2.32E−02 |
| Advanced glycosylation end product- | AGER | Q15109 | −0.46 | −0.74 | −0.17 | 1.51E−03 | 4.38E−02 |
| specific receptor, soluble | | | | | | | |
| Cysteine-rich with EGF-like domain | CRELD1 | Q96HD1 | −0.79 | −1.23 | −0.35 | 4.33E−04 | 1.80E−02 |
| protein 1 | | | | | | | |
| Protein kinase C-binding protein NELL1 | NELL1 | Q92832 | −0.89 | −1.44 | −0.33 | 1.75E−03 | 4.79E−02 |
| Proteoglycan 3 | PRG3 | Q9Y2Y8 | −0.93 | −1.4 | −0.46 | 1.20E−04 | 7.83E−03 |
| Glypican-3 | GPC3 | P51654 | −0.95 | −1.49 | −0.4 | 6.69E−04 | 2.43E−02 |
| Neuronal pentraxin receptor | NPTXR | O95502 | −0.99 | −1.6 | −0.38 | 1.55E−03 | 4.42E−02 |
| Dual specificity protein phosphatase 13 | DUSP13 | Q6B8I1 | −1.04 | −1.66 | −0.42 | 1.00E−03 | 3.22E−02 |
| isoform A | | | | | | | |
| Sodium/potassium-transporting ATPase | ATP1B1 | P05026 | −1.09 | −1.62 | −0.57 | 4.08E−05 | 3.69E−03 |
| subunit beta-1 | | | | | | | |
| Ck-beta-8-1 | CCL23 | P55773 | −1.18 | −1.7 | −0.65 | 1.01E−05 | 1.67E−03 |
| Group XIIB secretory phospholipase | PLA2G12B | Q9BX93 | −1.2 | −1.91 | −0.5 | 8.18E−04 | 2.83E−02 |
| A2-like protein | | | | | | | |
| Secreted frizzled-related protein 3 | FRZB | Q92765 | −1.22 | −1.88 | −0.55 | 3.39E−04 | 1.55E−02 |
| Neurocan core protein | NCAN | O14594 | −1.25 | −1.92 | −0.59 | 2.19E−04 | 1.14E−02 |
| A disintegrin and metalloproteinase with | ADAMTS13 | Q76LX8 | −1.25 | −1.97 | −0.52 | 7.72E−04 | 2.72E−02 |
| thrombospondin motifs 13 | | | | | | | |
| Coiled-coil domain-containing protein | CCDC126 | Q96EE4 | −1.27 | −2.01 | −0.53 | 8.08E−04 | 2.81E−02 |
| 126 | | | | | | | |
| Cartilage intermediate layer protein 2 | CILP2 | Q8IUL8 | −1.28 | −1.97 | −0.59 | 2.80E−04 | 1.34E−02 |
| Myeloid zinc finger 1 | MZF1 | P28698 | −1.3 | −2.11 | −0.49 | 1.75E−03 | 4.79E−02 |
| Prostaglandin F2 receptor negative | PTGFRN | Q9P2B2 | −1.39 | −2.11 | −0.66 | 1.72E−04 | 9.50E−03 |
| regulator | | | | | | | |
| Cerebellin-4 | CBLN4 | Q9NTU7 | −1.43 | −2.11 | −0.76 | 3.17E−05 | 3.29E−03 |
| Interleukin-6 receptor subunit alpha | IL6R | P08887 | −1.45 | −2.17 | −0.72 | 9.06E−05 | 6.36E−03 |
| Anthrax toxin receptor 1 | ANTXR1 | Q9H6X2 | −1.47 | −2.22 | −0.73 | 1.09E−04 | 7.33E−03 |
| Voltage-dependent calcium channel | CACNA2D3 | Q8IZS8 | −1.47 | −2.2 | −0.73 | 9.05E−05 | 6.36E−03 |
| subunit alpha-2/delta-3 | | | | | | | |
| Apolipoprotein M | APOM | O95445 | −1.49 | −2.43 | −0.55 | 1.82E−03 | 4.90E−02 |
| Beta-1,4-galactosyltransferase 6 | B4GALT6 | Q9UBX8 | −1.5 | −2.33 | −0.67 | 3.94E−04 | 1.70E−02 |
| Thrombin | F2 | P00734 | −1.53 | −2.29 | −0.77 | 7.42E−05 | 5.66E−03 |
| N-acetylglucosamine-1-phosphodiester | NAGPA | Q9UK23 | −1.55 | −2.5 | −0.6 | 1.41E−03 | 4.15E−02 |
| alpha-N-acetylglucosaminidase | | | | | | | |
| Cholinesterase | BCHE | P06276 | −1.56 | −2.46 | −0.67 | 6.38E−04 | 2.36E−02 |
| NAD-dependent protein deacetylase | SIRT3 | Q9NTG7 | −1.59 | −2.35 | −0.84 | 3.76E−05 | 3.65E−03 |
| sirtuin-3, mitochondrial | | | | | | | |
| Syntenin-1 | SDCBP | O00560 | −1.7 | −2.49 | −0.91 | 2.34E−05 | 2.78E−03 |
| Interleukin-1 receptor type 2 | IL1R2 | P27930 | −1.71 | −2.55 | −0.87 | 6.74E−05 | 5.32E−03 |
| GDNF family receptor alpha-2 | GFRA2 | O00451 | −1.78 | −2.8 | −0.77 | 5.74E−04 | 2.25E−02 |
| Receptor-type tyrosine-protein | PTPRJ | Q12913 | −1.78 | −2.81 | −0.76 | 6.46E−04 | 2.36E−02 |
| phosphatase eta | | | | | | | |
| Ciliary neurotrophic factor receptor | CNTFR | P26992 | −1.79 | −2.75 | −0.83 | 2.60E−04 | 1.28E−02 |
| subunit alpha | | | | | | | |
| Contactin-1 | CNTN1 | Q12860 | −1.8 | −2.88 | −0.72 | 1.07E−03 | 3.36E−02 |
| Leucine-rich repeats and | LRIG3 | Q6UXM1 | −1.8 | −2.82 | −0.78 | 5.41E−04 | 2.15E−02 |
| immunoglobulin-like domains protein 3 | | | | | | | |
| Golgi-associated plant pathogenesis- | GLIPR2 | Q9H4G4 | −1.86 | −2.85 | −0.86 | 2.72E−04 | 1.32E−02 |
| related protein 1 | | | | | | | |
| Heparan-sulfate 6-O-sulfotransferase 3 | HS6ST3 | Q8IZP7 | −1.86 | −3.02 | −0.69 | 1.79E−03 | 4.88E−02 |
| Matrix-remodeling-associated protein | MXRA8 | Q9BRK3 | −2.07 | −3 | −1.15 | 1.07E−05 | 1.67E−03 |
| 8: Extracellular domain | | | | | | | |
| Vitamin K-dependent protein C | PROC | P04070 | −2.09 | −3.32 | −0.86 | 8.62E−04 | 2.96E−02 |
| Glycosaminoglycan xylosylkinase | FAM20B | O75063 | −2.09 | −3.32 | −0.85 | 9.32E−04 | 3.12E−02 |
| Semaphorin-3G | SEMA3G | Q9NS98 | −2.15 | −3.13 | −1.16 | 1.93E−05 | 2.47E−03 |
| Collagen alpha-1(XIII) chain | COL13A1 | Q5TAT6 | −2.28 | −3.32 | −1.23 | 1.93E−05 | 2.47E−03 |
| Contactin-4 | CNTN4 | Q8IWV2 | −2.29 | −3.32 | −1.26 | 1.31E−05 | 1.82E−03 |
| B melanoma antigen 3 | BAGE3 | Q86Y29 | −2.29 | −3.47 | −1.12 | 1.33E−04 | 8.36E−03 |
| Afamin | AFM | P43652 | −2.53 | −3.67 | −1.4 | 1.20E−05 | 1.80E−03 |
| Neuropeptide S | NPS | P0C0P6 | −2.68 | −4.05 | −1.31 | 1.32E−04 | 8.36E−03 |
| Alpha-2-HS-glycoprotein | AHSG | P02765 | −3.19 | −4.71 | −1.67 | 3.83E−05 | 3.65E−03 |
| Epidermal growth factor receptor | EGFR | P00533 | −3.4 | −4.8 | −2 | 2.08E−06 | 5.98E−04 |

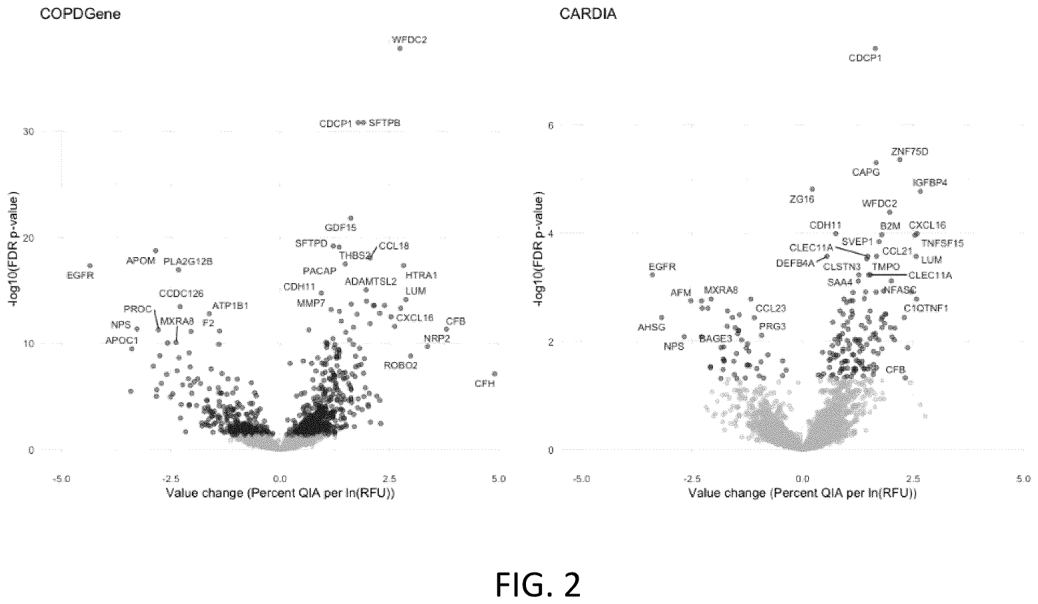

Of these, 144 proteins (143 GeneIDs) had associations with QIA that overlapped in COPDGene and CARDIA (FIG. 2 Table 6).

TABLE 6
Significant multivariable associations of proteomics with QIA in the COPDGene and CARDIA cohorts

| | | | COPDGene | | CARDIA | |
|---|---|---|---|---|---|---|
| | | | Percent QIA | | Percent QIA | |
| | | | per ln(RFU), | | per ln(RFU), | |
| Protein | Entrez | UniProt | Value | FDR p- | Value | FDR p- |
| name | Gene Name | ID | [95% CI] | value | [95% CI] | value |
| Complement | CFB | P00751 | 3.8 | 4.67E−12 | 2.32 | 4.77E−02 |
| factor B | | | [2.82 to 4.78] | | [0.87 to 3.77] | |
| Neuropilin-2 | NRP2 | O60462 | 3.37 | 2.02E−10 | 2.37 | 1.32E−02 |
| | | | [2.43 to 4.31] | | [1.09 to 3.64] | |
| Lumican | LUM | P51884 | 2.87 | 7.36E−15 | 2.56 | 2.70E−04 |
| | | | [2.21 to 3.54] | | [1.55 to 3.58] | |
| Spondin-1 | SPON1 | Q9HCB6 | 2.76 | 4.90E−14 | 1.6 | 3.52E−02 |
| | | | [2.1 to 3.41] | | [0.64 to 2.57] | |
| WAP four- | WFDC2 | Q14508 | 2.74 | 1.53E−38 | 1.96 | 4.16E−05 |
| disulfide | | | [2.35 to 3.13] | | [1.26 to 2.67] | |
| core domain | | | | | | |
| protein 2 | | | | | | |
| C-X-C motif | CXCL16 | Q9H2A7 | 2.62 | 2.57E−12 | 2.58 | 1.04E−04 |
| chemokine 16 | | | [1.95 to 3.28] | | [1.62 to 3.54] | |
| Neurofascin | NFASC | O94856 | 2.53 | 3.08E−13 | 2.45 | 1.22E−03 |
| | | | [1.91 to 3.15] | | [1.39 to 3.51] | |
| EGF- | EFEMP1 | Q12805 | 2.38 | 2.75E−14 | 1.79 | 1.39E−02 |
| containing | | | [1.82 to 2.94] | | [0.82 to 2.76] | |
| fibulin-like | | | | | | |
| extracellular | | | | | | |
| matrix | | | | | | |
| protein 1 | | | | | | |
| Leukocyte | LILRA5 | A6NI73 | 2.28 | 1.49E−13 | 1.53 | 8.95E−03 |
| immunoglobulin- | | | [1.72 to 2.83] | | [0.74 to 2.31] | |
| like receptor | | | | | | |
| subfamily A | | | | | | |
| member 5 | | | | | | |
| Serine/arginine- | SRSF6 | Q13247 | 2.14 | 2.75E−14 | 1.66 | 1.25E−03 |
| rich splicing | | | [1.64 to 2.65] | | [0.94 to 2.37] | |
| factor 6 | | | | | | |
| Collagen | COL28A1 | Q2UY09 | 2.13 | 2.70E−14 | 1.85 | 3.21E−03 |
| alpha- | | | [1.63 to 2.63] | | [0.98 to 2.72] | |
| 1(XXVIII) | | | | | | |
| chain | | | | | | |
| C-C motif | CCL18 | P55774 | 2.06 | 9.18E−19 | 0.99 | 4.51E−02 |
| chemokine 18 | | | [1.64 to 2.48] | | [0.38 to 1.61] | |
| Collagen | COL6A3 | P12111 | 2.05 | 1.41E−12 | 1.78 | 3.99E−03 |
| alpha-3(VI) | | | [1.54 to 2.57] | | [0.92 to 2.63] | |
| chain: Bovine | | | | | | |
| pancreatic | | | | | | |
| trypsin inhibitor/ | | | | | | |
| Kunitz inhibitor | | | | | | |
| domain, isoform 1 | | | | | | |
| Complement | C1QTNF1 | Q9BXJ1 | 2.03 | 5.35E−06 | 2.57 | 1.67E−03 |
| C1q tumor | | | [1.27 to 2.79] | | [1.43 to 3.71] | |
| necrosis factor- | | | | | | |
| related protein 1 | | | | | | |
| Lipopolysac | LBP | P18428 | 2.01 | 5.98E−05 | 1 | 9.77E−03 |
| charide- | | | [1.17 to 2.84] | | [0.48 to 1.53] | |
| binding protein | | | | | | |
| Beta-2- | B2M | P61769 | 1.97 | 1.09E−14 | 1.78 | 1.09E−04 |
| microglobulin | | | [1.52 to 2.43] | | [1.11 to 2.45] | |
| ADAMTS- | ADAMTSL2 | Q86TH1 | 1.96 | 9.26E−16 | 1.34 | 2.36E−02 |
| like protein 2 | | | [1.52 to 2.4] | | [0.57 to 2.11] | |
| Tumor necrosis | TNFSF15 | 095150 | 1.85 | 9.62E−09 | 2.54 | 1.11E−04 |
| factor ligand | | | [1.28 to 2.41] | | [1.58 to 3.49] | |
| superfamily | | | | | | |
| member 15 | | | | | | |
| Cystatin-C | CST3 | P01034 | 1.78 | 1.23E−08 | 1.71 | 9.51E−03 |
| | | | [1.24 to 2.33] | | [0.82 to 2.61] | |
| CUB domain- | CDCP1 | Q9H5V8 | 1.78 | 1.54E−31 | 1.64 | 3.92E−08 |
| containing | | | [1.5 to 2.06] | | [1.17 to 2.11] | |
| protein 1 | | | | | | |
| Triggering | TREM1 | Q9NP99 | 1.76 | 1.85E−13 | 1.39 | 1.64E−03 |
| receptor | | | [1.33 to 2.19] | | [0.78 to 2] | |
| expressed on | | | | | | |
| myeloid cells 1 | | | | | | |
| Gamma- | GGH | Q92820 | 1.72 | 1.69E−08 | 1.44 | 1.17E−02 |
| glutamyl | | | [1.19 to 2.26] | | [0.68 to 2.21] | |
| hydrolase | | | | | | |
| Peptidyl- | PPIC | P45877 | 1.71 | 1.37E−07 | 1.59 | 1.56E−02 |
| prolyl cis- | | | [1.14 to 2.27] | | [0.72 to 2.47] | |
| trans | | | | | | |
| isomerase C | | | | | | |
| C-C motif | CCL21 | O00585 | 1.68 | 3.08E−12 | 1.67 | 2.70E−04 |
| chemokine 21 | | | [1.25 to 2.1] | | [1 to 2.33] | |
| Sushi, von | SVEP1 | Q4LDE5 | 1.62 | 1.98E−14 | 1.73 | 1.46E−04 |
| Willebrand | | | [1.24 to 2] | | [1.07 to 2.39] | |
| factor type | | | | | | |
| A, EGF and | | | | | | |
| pentraxin | | | | | | |
| domain- | | | | | | |
| containing | | | | | | |
| protein 1: | | | | | | |
| EGF-like | | | | | | |
| domains 4-6 | | | | | | |
| Vesicular | LMAN2 | Q12907 | 1.62 | 1.89E−06 | 1.6 | 2.70E−02 |
| integral- | | | [1.03 to 2.2] | | [0.67 to 2.53] | |
| membrane | | | | | | |
| protein VIP36 | | | | | | |
| Growth/ | GDF15 | Q99988 | 1.61 | 1.54E−22 | 0.84 | 3.80E−02 |
| differentiation | | | [1.31 to 1.91] | | [0.33 to 1.35] | |
| factor 15 | | | | | | |
| Retinoic acid | RARRES2 | Q99969 | 1.59 | 1.04E−06 | 1.39 | 3.27E−02 |
| receptor | | | [1.03 to 2.15] | | [0.56 to 2.21] | |
| responder | | | | | | |
| protein 2 | | | | | | |
| Axin-2 | AXIN2 | Q9Y2T1 | 1.52 | 1.64E−07 | 0.91 | 3.19E−03 |
| | | | [1.02 to 2.03] | | [0.48 to 1.33] | |
| Sushi, von | SVEP1 | Q4LDE5 | 1.5 | 9.39E−12 | 1.51 | 4.40E−03 |
| Willebrand | | | [1.11 to 1.89] | | [0.78 to 2.24] | |
| factor type | | | | | | |
| A, EGF and | | | | | | |
| pentraxin | | | | | | |
| domain- | | | | | | |
| containing | | | | | | |
| protein 1: | | | | | | |
| Sushi 15-18 | | | | | | |
| Marginal | MZB1 | Q8WU39 | 1.49 | 3.30E−18 | 1.25 | 7.81E−04 |
| zone B- and | | | [1.18 to 1.8] | | [0.73 to 1.78] | |
| B1-cell- | | | | | | |
| specific protein | | | | | | |
| Plexin-D1 | PLXND1 | Q9Y4D7 | 1.47 | 1.90E−05 | 1.51 | 2.99E−02 |
| | | | [0.89 to 2.05] | | [0.62 to 2.4] | |
| Complement C2 | C2 | P06681 | 1.46 | 1.26E−03 | 1.98 | 5.66E−03 |
| | | | [0.73 to 2.18] | | [1 to 2.96] | |
| Ganglioside | GM2A | P17900 | 1.45 | 2.16E−07 | 1.59 | 7.47E−03 |
| GM2 activator | | | [0.97 to 1.94] | | [0.78 to 2.4] | |
| Antileukoproteinase | SLPI | P03973 | 1.41 | 5.91E−04 | 2.07 | 9.49E−03 |
| | | | [0.74 to 2.08] | | [0.99 to 3.15] | |
| E-selectin | SELE | P16581 | 1.4 | 7.89E−13 | 1.02 | 6.00E−03 |
| | | | [1.05 to 1.74] | | [0.51 to 1.53] | |
| C-X-C motif | CXCL13 | O43927 | 1.35 | 9.68E−14 | 1.42 | 1.24E−03 |
| chemokine 13 | | | [1.03 to 1.68] | | [0.81 to 2.04] | |
| Tumor necrosis | TNFRSF1B | P20333 | 1.34 | 1.94E−09 | 1.25 | 9.49E−03 |
| factor receptor | | | [0.95 to 1.73] | | [0.6 to 1.9] | |
| superfamily | | | | | | |
| member 1B | | | | | | |
| Thrombospondin-2 | THBS2 | P35442 | 1.34 | 8.28E−20 | 0.91 | 1.41E−02 |
| | | | [1.08 to 1.61] | | [0.42 to 1.41] | |
| Macrophage- | CAPG | P40121 | 1.29 | 1.82E−09 | 1.66 | 5.08E−06 |
| capping protein | | | [0.91 to 1.66] | | [1.11 to 2.21] | |
| Tumor necrosis | TNFRSF17 | Q02223 | 1.29 | 1.69E−06 | 1.19 | 3.43E−02 |
| factor receptor | | | [0.83 to 1.75] | | [0.48 to 1.91] | |
| superfamily | | | | | | |
| member 17 | | | | | | |
| Serine/threonine- | PPP4R3A | Q6IN85 | 1.29 | 2.57E−09 | 1.15 | 1.79E−02 |
| protein | | | [0.91 to 1.67] | | [0.51 to 1.78] | |
| phosphatase | | | | | | |
| 4 regulatory | | | | | | |
| subunit 3A | | | | | | |
| Neuroblastoma | NBL1 | P41271 | 1.27 | 1.87E−09 | 1.42 | 5.82E−03 |
| suppressor of | | | [0.9 to 1.65] | | [0.71 to 2.12] | |
| tumorigenicity 1 | | | | | | |
| WAP four- | WFDC1 | Q9HC57 | 1.27 | 7.15E−07 | 1.14 | 3.86E−02 |
| disulfide | | | [0.83 to 1.71] | | [0.45 to 1.84] | |
| core domain | | | | | | |
| protein 1 | | | | | | |
| Golgi membrane | GOLM1 | Q8NBJ4 | 1.25 | 1.38E−10 | 1.04 | 1.40E−02 |
| protein 1 | | | [0.9 to 1.59] | | [0.47 to 1.6] | |
| Thioredoxin | TXNDC5 | Q8NBS9 | 1.24 | 1.88E−07 | 1.38 | 3.22E−02 |
| domain- | | | [0.82 to 1.65] | | [0.56 to 2.2] | |
| containing | | | | | | |
| protein 5 | | | | | | |
| C-C motif | CCL3 | P10147 | 1.23 | 9.93E−08 | 0.87 | 3.22E−02 |
| chemokine 3 | | | [0.83 to 1.63] | | [0.35 to 1.39] | |
| Calsyntenin-3 | CLSTN3 | Q9BQT9 | 1.22 | 9.19E−08 | 1.45 | 3.04E−04 |
| | | | [0.82 to 1.61] | | [0.87 to 2.03] | |
| Matrix Gla | MGP | P08493 | 1.21 | 6.95E−04 | 2.08 | 5.78E−03 |
| protein | | | [0.63 to 1.79] | | [1.05 to 3.11] | |
| Leucine-rich | LRIG1 | Q96JA1 | 1.2 | 9.14E−04 | 1.8 | 3.65E−03 |
| repeats and | | | [0.61 to 1.78] | | [0.94 to 2.65] | |
| immunoglobulin- | | | | | | |
| like domains | | | | | | |
| protein 1 | | | | | | |
| Ephrin type- | EPHA2 | P29317 | 1.2 | 7.07E−06 | 1.33 | 2.81E−02 |
| A receptor 2 | | | [0.75 to 1.65] | | [0.55 to 2.11] | |
| IGF-like | IGFLR1 | Q9H665 | 1.13 | 2.37E−09 | 1.08 | 3.65E−03 |
| family receptor 1 | | | [0.8 to 1.46] | | [0.57 to 1.6] | |
| Low-density | LRP11 | Q86VZ4 | 1.12 | 4.89E−03 | 1.79 | 5.66E−03 |
| lipoprotein | | | [0.5 to 1.75] | | [0.9 to 2.67] | |
| receptor- | | | | | | |
| related protein 11 | | | | | | |
| Ribonuclease K6 | RNASE6 | Q93091 | 1.12 | 1.97E−07 | 1.33 | 5.11E−03 |
| | | | [0.75 to 1.49] | | [0.68 to 1.98] | |
| Interleukin | ILF3 | Q12906 | 1.09 | 1.65E−03 | 1.65 | 8.62E−03 |
| enhancer- | | | [0.53 to 1.64] | | [0.8 to 2.49] | |
| binding factor 3 | | | | | | |
| Macrophage | MMP12 | P39900 | 1.06 | 2.40E−10 | 1.14 | 1.27E−03 |
| metalloelastase | | | [0.76 to 1.35] | | [0.64 to 1.63] | |
| Elafin | PI3 | P19957 | 1.04 | 9.71E−09 | 1.01 | 4.47E−03 |
| | | | [0.72 to 1.36] | | [0.52 to 1.49] | |
| BPI fold- | BPIFB1 | Q8TDL5 | 1.04 | 7.97E−11 | 0.68 | 1.75E−02 |
| containing | | | [0.76 to 1.32] | | [0.3 to 1.06] | |
| family B | | | | | | |
| member 1 | | | | | | |
| Erythropoietin | EPO | P01588 | 1.03 | 5.37E−08 | 0.99 | 1.94E−03 |
| | | | [0.7 to 1.35] | | [0.54 to 1.44] | |
| Interleukin- | IL3RA | P26951 | 1.03 | 3.59E−06 | 0.95 | 4.70E−02 |
| 3 receptor | | | [0.65 to 1.4] | | [0.36 to 1.54] | |
| subunit alpha | | | | | | |
| Fibulin-5 | FBLN5 | Q9UBX5 | 1.02 | 7.87E−04 | 2.29 | 3.66E−03 |
| | | | [0.53 to 1.51] | | [1.2 to 3.39] | |
| Kallikrein-10 | KLK10 | O43240 | 1.02 | 5.17E−06 | 1.12 | 1.79E−02 |
| | | | [0.64 to 1.41] | | [0.5 to 1.75] | |
| Insulin-like | IGFBP4 | P22692 | 1.01 | 1.10E−02 | 2.66 | 1.71E−05 |
| growth factor- | | | [0.4 to 1.62] | | [1.74 to 3.58] | |
| binding protein 4 | | | | | | |
| Intercellular | ICAM1 | P05362 | 1 | 3.25E−07 | 1.13 | 3.21E−03 |
| adhesion molecule 1 | | | [0.66 to 1.34] | | [0.6 to 1.66] | |
| Transmembrane | TMED10 | P49755 | 0.98 | 1.30E−04 | 1.21 | 2.26E−02 |
| emp24 domain- | | | [0.56 to 1.41] | | [0.52 to 1.9] | |
| containing | | | | | | |
| protein 10 | | | | | | |
| Protein | CCN1 | O00622 | 0.97 | 3.07E−04 | 1.18 | 9.24E−03 |
| CYR61 | | | [0.53 to 1.41] | | [0.57 to 1.79] | |
| B melanoma | BAGE2 | Q86Y30 | 0.96 | 2.09E−04 | 1.82 | 1.19E−03 |
| antigen 2 | | | [0.54 to 1.39] | | [1.04 to 2.61] | |
| Serpin B8 | SERPINB8 | P50452 | 0.96 | 6.38E−03 | 1.64 | 7.28E−03 |
| | | | [0.41 to 1.51] | | [0.81 to 2.46] | |
| Phospholipase | PLA2G2A | P14555 | 0.96 | 7.49E−08 | 0.88 | 2.54E−02 |
| A2, membrane | | | [0.65 to 1.27] | | [0.37 to 1.39] | |
| associated | | | | | | |
| C-C motif | CCL22 | O00626 | 0.95 | 2.11E−06 | 1.19 | 9.93E−03 |
| chemokine 22 | | | [0.61 to 1.29] | | [0.57 to 1.82] | |
| Thrombospondin-3 | THBS3 | P49746 | 0.94 | 7.19E−04 | 1.29 | 9.49E−03 |
| | | | [0.49 to 1.39] | | [0.62 to 1.97] | |
| Cadherin-11: | CDH11 | P55287 | 0.94 | 1.86E−15 | 0.75 | 1.04E−04 |
| Extracellular | | | [0.72 to 1.15] | | [0.47 to 1.02] | |
| domain | | | | | | |
| Alpha-1- | SERPINA3 | P01011 | 0.88 | 6.97E−04 | 1.28 | 1.56E−02 |
| antichymotrypsin | | | [0.46 to 1.3] | | [0.58 to 1.99] | |
| complex | | | | | | |
| Lysozyme C | LYZ | P61626 | 0.86 | 5.58E−04 | 1.01 | 3.18E−02 |
| | | | [0.45 to 1.26] | | [0.41 to 1.6] | |
| SLAM family | SLAMF7 | Q9NQ25 | 0.86 | 2.37E−09 | 0.73 | 9.29E−03 |
| member 7 | | | [0.6 to 1.11] | | [0.35 to 1.1] | |
| Troponin T, | TNNT2 | P45379 | 0.85 | 1.37E−04 | 1.13 | 1.80E−03 |
| cardiac muscle | | | [0.48 to 1.22] | | [0.62 to 1.64] | |
| Fibroblast | FGF23 | Q9GZV9 | 0.85 | 1.11E−05 | 0.84 | 9.24E−03 |
| growth factor 23 | | | [0.52 to 1.18] | | [0.4 to 1.27] | |
| Trafficking | TRAPPC3 | O43617 | 0.83 | 3.26E−04 | 1.25 | 2.36E−02 |
| protein particle | | | [0.45 to 1.21] | | [0.53 to 1.97] | |
| complex subunit 3 | | | | | | |
| Oncoprotein- | OIT3 | Q8WWZ8 | 0.8 | 2.34E−03 | 1.31 | 1.23E−02 |
| induced | | | [0.38 to 1.22] | | [0.61 to 2.01] | |
| transcript 3 | | | | | | |
| protein | | | | | | |
| Cerebellin-4 | CBLN4 | Q9NTU7 | 0.8 | 1.04E−02 | −1.43 | 3.29E−03 |
| | | | [0.32 to 1.28] | | [−2.11 to −0.76] | |
| Nidogen-1 | NID1 | P14543 | 0.79 | 1.32E−02 | 1.88 | 3.19E−03 |
| | | | [0.3 to 1.28] | | [1 to 2.76] | |
| C-X-C motif | CXCL10 | P02778 | 0.79 | 2.55E−05 | 1.08 | 1.80E−03 |
| chemokine 10 | | | [0.47 to 1.1] | | [0.59 to 1.56] | |
| Protein | ABHD14A | Q9BUJ0 | 0.77 | 1.07E−03 | 1.47 | 3.65E−03 |
| ABHD14A | | | [0.39 to 1.15] | | [0.77 to 2.16] | |
| Ribonuclease | RNASE1 | P07998 | 0.74 | 2.81E−07 | 0.79 | 1.10E−02 |
| pancreatic | | | [0.49 to 0.99] | | [0.37 to 1.21] | |
| Vascular | VEGFD | O43915 | 0.71 | 8.97E−04 | 0.93 | 5.19E−03 |
| endothelial growth | | | [0.37 to 1.06] | | [0.47 to 1.38] | |
| factor D | | | | | | |
| Serum amyloid | SAA2 | PODJI9 | 0.71 | 8.00E−09 | 0.67 | 1.22E−02 |
| A-2 protein | | | [0.5 to 0.93] | | [0.31 to 1.03] | |
| RNA-binding | EWSR1 | Q01844 | 0.7 | 2.37E−02 | 1.7 | 1.70E−02 |
| protein EWS | | | [0.24 to 1.17] | | [0.76 to 2.64] | |
| C-reactive | CRP | P02741 | 0.65 | 5.26E−12 | 0.45 | 1.61E−02 |
| protein | | | [0.48 to 0.82] | | [0.2 to 0.69] | |
| Thiosulfate | TST | Q16762 | 0.64 | 7.70E−05 | 0.74 | 1.83E−02 |
| sulfurtransferase | | | [0.37 to 0.91] | | [0.33 to 1.16] | |
| Syntaxin-12 | STX12 | Q86Y82 | 0.63 | 3.13E−03 | 0.95 | 2.36E−02 |
| | | | [0.29 to 0.97] | | [0.41 to 1.5] | |
| Ubiquitin- | ISG15 | P05161 | 0.61 | 7.10E−05 | 0.84 | 2.78E−03 |
| like protein | | | [0.35 to 0.86] | | [0.45 to 1.23] | |
| ISG15 | | | | | | |
| Lamina- | TMPO | P42167 | 0.6 | 7.07E−03 | 1.49 | 5.98E−04 |
| associated | | | [0.25 to 0.95] | | [0.88 to 2.11] | |
| polypeptide 2, | | | | | | |
| isoforms beta/ | | | | | | |
| gamma | | | | | | |
| DnaJ homolog | DNAJB12 | Q9NXW2 | 0.55 | 3.65E−03 | 1.09 | 4.51E−02 |
| subfamily B | | | [0.25 to 0.84] | | [0.41 to 1.76] | |
| member 12 | | | | | | |
| C-X-C motif | CXCL11 | O14625 | 0.55 | 4.79E−06 | 0.47 | 4.02E−02 |
| chemokine 11 | | | [0.35 to 0.76] | | [0.18 to 0.75] | |
| Solute carrier | SLCO5A1 | Q9H2Y9 | 0.54 | 4.73E−02 | 0.76 | 4.90E−02 |
| organic anion | | | [0.14 to 0.94] | | [0.28 to 1.24] | |
| transporter | | | | | | |
| family member | | | | | | |
| 5A1 | | | | | | |
| Gastrokine-2 | GKN2 | Q86XP6 | 0.54 | 3.18E−03 | 0.72 | 2.02E−02 |
| | | | [0.25 to 0.83] | | [0.31 to 1.13] | |
| Thioredoxin | TXNRD1 | Q16881 | 0.53 | 5.73E−03 | 0.96 | 2.36E−02 |
| reductase 1, | | | [0.23 to 0.84] | | [0.41 to 1.51] | |
| cytoplasmic | | | | | | |
| Lactoperoxidase | LPO | P22079 | 0.53 | 1.07E−02 | 0.93 | 1.67E−03 |
| | | | [0.21 to 0.86] | | [0.52 to 1.35] | |
| Troponin I, | TNNI2 | P48788 | 0.52 | 1.46E−04 | 0.6 | 2.25E−02 |
| fast skeletal | | | [0.29 to 0.75] | | [0.26 to 0.94] | |
| muscle | | | | | | |
| Serum amyloid | SAA1 | PODJI8 | 0.52 | 4.60E−09 | 0.41 | 4.51E−02 |
| A-1 protein | | | [0.37 to 0.68] | | [0.15 to 0.66] | |
| C-C motif | CCL19 | Q99731 | 0.45 | 2.42E−02 | 0.9 | 1.56E−02 |
| chemokine 19 | | | [0.15 to 0.75] | | [0.41 to 1.39] | |
| Gamma- | IFI16 | Q16666 | 0.45 | 9.00E−03 | 0.87 | 3.43E−02 |
| interferon- | | | [0.18 to 0.72] | | [0.35 to 1.39] | |
| inducible | | | | | | |
| protein | | | | | | |
| 16: Isoform 2, | | | | | | |
| Hematopoietic | | | | | | |
| expression, | | | | | | |
| interferon- | | | | | | |
| inducible nature, | | | | | | |
| and nuclear | | | | | | |
| localization 1 | | | | | | |
| Granulocyte | CSF3 | P09919 | 0.45 | 1.49E−02 | 0.82 | 8.03E−03 |
| colony- | | | [0.17 to 0.73] | | [0.4 to 1.25] | |
| stimulating factor | | | | | | |
| Tapasin | TAPBP | O15533 | 0.45 | 1.28E−02 | 0.61 | 1.65E−02 |
| | | | [0.17 to 0.73] | | [0.27 to 0.95] | |
| Chitotriosidase-1 | CHIT1 | Q13231 | 0.44 | 6.36E−04 | 0.62 | 2.15E−02 |
| | | | [0.23 to 0.65] | | [0.27 to 0.97] | |
| Interferon- | MX1 | P20591 | 0.42 | 1.24E−02 | 0.9 | 7.33E−03 |
| induced GTP- | | | [0.16 to 0.68] | | [0.44 to 1.35] | |
| binding | | | | | | |
| protein Mx1 | | | | | | |
| Beta- | DEFB4A | O15263 | 0.37 | 9.34E−06 | 0.55 | 2.70E−04 |
| defensin 4A | | | [0.23 to 0.51] | | [0.33 to 0.77] | |
| Chitinase-3- | CHI3L1 | P36222 | 0.22 | 1.36E−02 | 0.35 | 4.23E−02 |
| like protein 1 | | | [0.08 to 0.35] | | [0.14 to 0.57] | |
| Zymogen | ZG16 | O60844 | 0.22 | 7.92E−09 | 0.21 | 1.56E−05 |
| granule | | | [0.16 to 0.29] | | [0.14 to 0.29] | |
| membrane | | | | | | |
| protein 16 | | | | | | |
| Advanced | AGER | Q15109 | −0.55 | 8.74E−06 | −0.46 | 4.38E−02 |
| glycosylation | | | [−0.77 to −0.34] | | [−0.74 to −0.17] | |
| end product- | | | | | | |
| specific | | | | | | |
| receptor, | | | | | | |
| soluble | | | | | | |
| Protein | NELL1 | Q92832 | −0.55 | 1.08E−02 | −0.89 | 4.79E−02 |
| kinase C- | | | [−0.88 to −0.22] | | [−1.44 to −0.33] | |
| binding | | | | | | |
| protein NELL1 | | | | | | |
| Cartilage | CILP2 | Q8IUL8 | −0.57 | 3.61E−02 | −1.28 | 1.34E−02 |
| intermediate | | | [−0.98 to −0.17] | | [−1.97 to −0.59] | |
| layer protein 2 | | | | | | |
| Glypican-3 | GPC3 | P51654 | −0.59 | 2.06E−02 | −0.95 | 2.43E−02 |
| | | | [−0.98 to −0.21] | | [−1.49 to −0.4] | |
| N-acetylglucos | NAGPA | Q9UK23 | −0.79 | 2.11E−02 | −1.55 | 4.15E−02 |
| amine-1- | | | [−1.3 to −0.27] | | [−2.5 to −0.6] | |
| phosphodiester | | | | | | |
| alpha-N- | | | | | | |
| acetylglucos- | | | | | | |
| aminidase | | | | | | |
| Prostaglandin | PTGFRN | Q9P2B2 | −0.83 | 8.36E−03 | −1.39 | 9.50E−03 |
| F2 receptor | | | [−1.32 to −0.341 | | [−2.11 to −0.66] | |
| negative regulator | | | | | | |
| Beta-1,4- | B4GALT6 | Q9UBX8 | −0.83 | 1.55E−02 | −1.5 | 1.70E−02 |
| galactosyl- | | | [−1.35 to −0.31] | | [−2.33 to −0.67] | |
| transferase 6 | | | | | | |
| Voltage- | CACNA2D3 | Q8IZS8 | −0.93 | 3.88E−03 | −1.47 | 6.36E−03 |
| dependent | | | [−1.43 to −0.42] | | [−2.2 to −0.73] | |
| calcium channel | | | | | | |
| subunit alpha- | | | | | | |
| 2/delta-3 | | | | | | |
| Interleukin-1 | IL1R2 | P27930 | −0.98 | 7.54E−03 | −1.71 | 5.32E−03 |
| receptor type 2 | | | [−1.56 to −0.41] | | [−2.55 to −0.87] | |
| Golgi- | GLIPR2 | Q9H4G4 | −1 | 1.00E−02 | −1.86 | 1.32E−02 |
| associated plant | | | [−1.6 to −0.4] | | [−2.85 to −0.86] | |
| pathogenesis- | | | | | | |
| related protein 1 | | | | | | |
| Anthrax | ANTXR1 | Q9H6X2 | −1.14 | 6.53E−04 | −1.47 | 7.33E−03 |
| toxin receptor 1 | | | [−1.68 to −0.6] | | [−2.22 to −0.73] | |
| Neurocan | NCAN | O14594 | −1.2 | 2.25E−07 | −1.25 | 1.14E−02 |
| core protein | | | [−1.6 to −0.8] | | [−1.92 to −0.59] | |
| Contactin-4 | CNTN4 | Q8IWV2 | −1.34 | 3.73E−03 | −2.29 | 1.82E−03 |
| | | | [−2.07 to −0.62] | | [−3.32 to −1.26] | |
| Semaphorin-3G | SEMA3G | Q9NS98 | −1.38 | 1.07E−03 | −2.15 | 2.47E−03 |
| | | | [−2.06 to −0.7] | | [−3.13 to −1.16] | |
| Thrombin | F2 | P00734 | −1.39 | 6.92E−12 | −1.53 | 5.66E−03 |
| | | | [−1.75 to −1.03] | | [−2.29 to −0.77] | |
| Leucine-rich | LRIG3 | Q6UXM1 | −1.39 | 3.03E−05 | −1.8 | 2.15E−02 |
| repeats and | | | [−1.96 to −0.83] | | [−2.82 to −0.78] | |
| immunoglobulin- | | | | | | |
| like domains | | | | | | |
| protein 3 | | | | | | |
| Interleukin-6 | IL6R | P08887 | −1.42 | 1.61E−06 | −1.45 | 6.36E−03 |
| receptor subunit | | | [−1.92 to −0.91] | | [−2.17 to −0.72] | |
| alpha | | | | | | |
| A disintegrin | ADAMTS13 | Q76LX8 | −1.58 | 2.60E−08 | −1.25 | 2.72E−02 |
| and | | | [−2.08 to −1.09] | | [−1.97 to −0.52] | |
| metalloproteinase | | | | | | |
| with | | | | | | |
| thrombospondin | | | | | | |
| motifs 13 | | | | | | |
| Sodium/ | ATP1B1 | P05026 | −1.63 | 1.64E−13 | −1.09 | 3.69E−03 |
| potassium- | | | [−2.02 to −1.23] | | [−1.62 to −0.57] | |
| transporting | | | | | | |
| ATPase | | | | | | |
| subunit beta-1 | | | | | | |
| Cholinesterase | BCHE | P06276 | −1.63 | 1.98E−06 | −1.56 | 2.36E−02 |
| | | | [−2.21 to −1.04] | | [−2.46 to −0.67] | |
| Alpha-2-HS- | AHSG | P02765 | −1.64 | 1.52E−03 | −3.19 | 3.65E−03 |
| glycoprotein | | | [−2.47 to −0.81] | | [−4.71 to −1.67] | |
| Collagen | COL13A1 | Q5TAT6 | −1.89 | 2.21E−07 | −2.28 | 2.47E−03 |
| alpha- | | | [−2.52 to −1.26] | | [−3.32 to −1.23] | |
| 1(XIII) chain | | | | | | |
| Afamin | AFM | P43652 | −2.06 | 9.49E−07 | −2.53 | 1.80E−03 |
| | | | [−2.78 to −1.34] | | [−3.67 to −1.4] | |
| Receptor- | PTPRJ | Q12913 | −2.1 | 2.15E−07 | −1.78 | 2.36E−02 |
| type tyrosine- | | | [−2.8 to −1.4] | | [−2.81 to −0.76] | |
| protein | | | | | | |
| phosphatase eta | | | | | | |
| Coiled-coil | CCDC126 | Q96EE4 | −2.29 | 3.55E−14 | −1.27 | 2.81E−02 |
| domain- | | | [−2.83 to −1.75] | | [−2.01 to −0.53] | |
| containing | | | | | | |
| protein 126 | | | | | | |
| Ciliary | CNTFR | P26992 | −2.32 | 2.39E−09 | −1.79 | 1.28E−02 |
| neurotrophic | | | [−3 to −1.64] | | [−2.75 to −0.83] | |
| factor | | | | | | |
| receptor | | | | | | |
| subunit alpha | | | | | | |
| Group XIIB | PLA2G12B | Q9BX93 | −2.33 | 1.16E−17 | −1.2 | 2.83E−02 |
| secretory | | | [−2.82 to −1.84] | | [−1.91 to −0.5] | |
| phospholipase | | | | | | |
| A2-like protein | | | | | | |
| Contactin-1 | CNTN1 | Q12860 | −2.36 | 3.95E−08 | −1.8 | 3.36E−02 |
| | | | [−3.11 to −1.61] | | [−2.88 to −0.72] | |
| Matrix- | MXRA8 | Q9BRK3 | −2.39 | 7.97E−11 | −2.07 | 1.67E−03 |
| remodeling- | | | [−3.04 to −1.74] | | [−3 to −1.15] | |
| associated | | | | | | |
| protein 8: | | | | | | |
| Extracellular | | | | | | |
| domain | | | | | | |
| Heparan- | HS6ST3 | Q8IZP7 | −2.46 | 5.90E−06 | −1.86 | 4.88E−02 |
| sulfate 6-O- | | | [−3.39 to −1.54] | | [−3.02 to −0.69] | |
| sulfotransferase 3 | | | | | | |
| B melanoma | BAGE3 | Q86Y29 | −2.76 | 1.47E−09 | −2.29 | 8.36E−03 |
| antigen 3 | | | [−3.56 to −1.96] | | [−3.47 to −1.12] | |
| Vitamin K- | PROC | P04070 | −2.79 | 5.26E−12 | −2.09 | 2.96E−02 |
| dependent | | | [−3.51 to −2.07] | | [−3.32 to −0.86] | |
| protein C | | | | | | |
| Apolipoprotein M | APOM | O95445 | −2.85 | 1.87E−19 | −1.49 | 4.90E−02 |
| | | | [−3.43 to −2.28] | | [−2.43 to −0.55] | |
| Neuropeptide S | NPS | P0C0P6 | −3.28 | 4.39E−12 | −2.68 | 8.36E−03 |
| | | | [−4.12 to −2.44] | | [−4.05 to −1.31] | |
| Epidermal | EGFR | P00533 | −4.35 | 4.73E−18 | −3.4 | 5.98E−04 |
| growth factor | | | [−5.26 to −3.44] | | [−4.8 to −2] | |
| receptor | | | | | | |

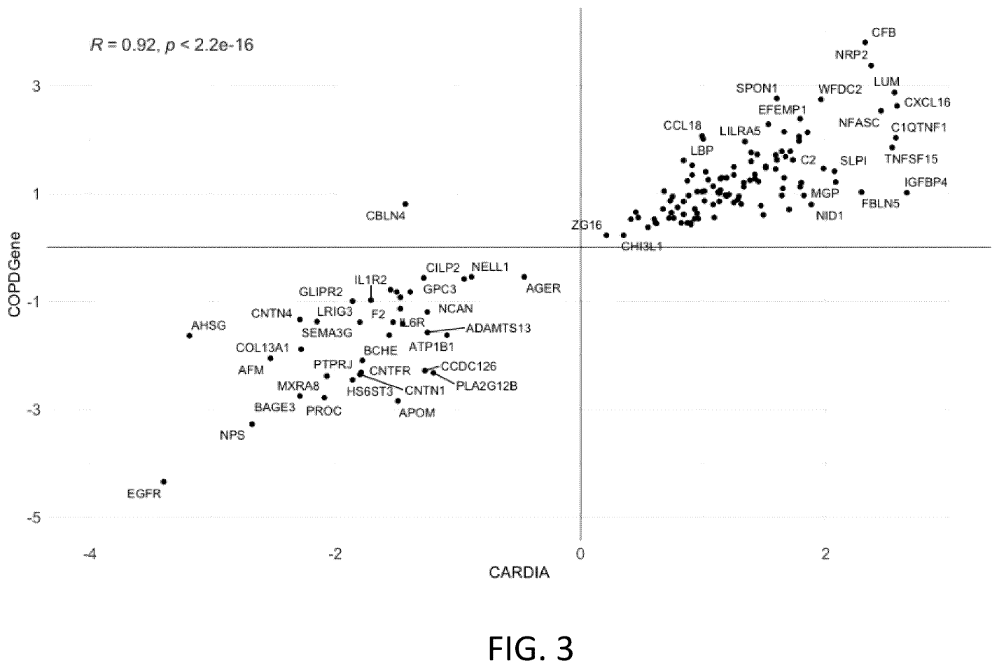

All but one of the 144 proteins shared directionality and magnitude between the two cohorts, with a Pearson correlation between regression coefficients across proteins of 0.92 and p<z2.2×10⁻¹⁶ (FIG. 3). Of the 144 proteins, 140 remained significantly associated with QIA and maintained directionality of association in both cohorts when adjusted for concurrent quantitative emphysema, another smoking-related CT phenotype (Table 7).

TABLE 7
Multivariable regression analysis of QIA in COPDGene and CARDIA, after adjusting for quantitative emphysema

| | | | COPDGene | | | | | CARDIA | | | | |
|---|---|---|---|---|---|---|---|---|---|---|---|---|
| | | | | Up- | Low- | | | | Up- | Low- | | |
| | Entrez | | Percent | per | er | | | Percent | per | er | | |
| | gene | UniProt | QIA per | 95% | 95% | | FDR p- | QIA per | 95% | 95% | | FDR p- |
| Protein name | name | ID | ln(RFU) | CI | CI | p-value | value | ln(RFU) | CI | CI | p-value | value |
| Neuropilin-2 | NRP2 | O60462 | 3.37 | 2.43 | 4.31 | 2.02E−12 | 2.01E−10 | 2.33 | 1.07 | 3.6 | 3.03E−04 | 1.44E−02 |
| Lumican | LUM | P51884 | 2.88 | 2.22 | 3.55 | 1.88E−17 | 6.24E−15 | 2.52 | 1.52 | 3.53 | 9.23E−07 | 2.87E−04 |
| Spondin- | SPON1 | Q9HCB6 | 2.76 | 2.1 | 3.41 | 2.42E−16 | 5.48E−14 | 1.52 | 0.57 | 2.48 | 1.85E−03 | 4.58E−02 |
| WAP four-disulfide | WFDC2 | Q14508 | 2.74 | 2.35 | 3.13 | 3.43E−42 | 1.71E−38 | 2.02 | 1.32 | 2.72 | 1.63E−08 | 2.02E−05 |
| core domain protein 2 | | | | | | | | | | | | |
| C-X-C motif | CXCL16 | Q9H2A7 | 2.62 | 1.95 | 3.29 | 1.79E−14 | 2.87E−12 | 2.5 | 1.54 | 3.45 | 3.27E−07 | 1.61E−04 |
| chemokine 16 | | | | | | | | | | | | |
| Neurofascin | NFASC | O94856 | 2.54 | 1.92 | 3.16 | 1.40E−15 | 2.48E−13 | 2.22 | 1.16 | 3.27 | 3.74E−05 | 3.44E−03 |
| EGF-containing | EFEMP1 | Q12805 | 2.38 | 1.82 | 2.94 | 1.12E−16 | 2.92E−14 | 1.84 | 0.88 | 2.8 | 1.76E−04 | 9.84E−03 |
| fibulin-like | | | | | | | | | | | | |
| extracellular matrix | | | | | | | | | | | | |
| protein 1 | | | | | | | | | | | | |
| Leukocyte | LILRA5 | A6NI73 | 2.28 | 1.73 | 2.83 | 8.21E−16 | 1.57E−13 | 1.47 | 0.69 | 2.26 | 2.28E−04 | 1.17E−02 |
| immunoglobulin-like | | | | | | | | | | | | |
| receptor subfamily A | | | | | | | | | | | | |
| member 5 | | | | | | | | | | | | |
| Serine/arginine-rich | SRSF6 | Q13247 | 2.15 | 1.64 | 2.65 | 1.17E−16 | 2.92E−14 | 1.67 | 0.96 | 2.39 | 4.59E−06 | 9.14E−04 |
| splicing factor 6 | | | | | | | | | | | | |
| Collagen alpha- | COL28A1 | Q2UY09 | 2.14 | 1.64 | 2.64 | 7.75E−17 | 2.14E−14 | 1.86 | 0.99 | 2.72 | 2.59E−05 | 2.74E−03 |
| 1(XXVIII) chain | | | | | | | | | | | | |
| Collagen alpha-3(VI) | COL6A3 | P12111 | 2.06 | 1.54 | 2.58 | 7.47E−15 | 1.24E−12 | 1.76 | 0.91 | 2.61 | 4.60E−05 | 3.95E−03 |
| chain: Bovine | | | | | | | | | | | | |
| pancreatic trypsin | | | | | | | | | | | | |
| inhibitor/Kunitz | | | | | | | | | | | | |
| inhibitor domain, | | | | | | | | | | | | |
| isoform 1 | | | | | | | | | | | | |
| C-C motif chemokine | CCL18 | P55774 | 2.06 | 1.64 | 2.48 | 1.65E−21 | 1.03E−18 | 1 | 0.38 | 1.61 | 1.44E−03 | 4.00E−02 |
| 18 | | | | | | | | | | | | |
| Complement C1q | C1QTNF1 | Q9BXJ1 | 2.03 | 1.27 | 2.79 | 1.69E−07 | 5.37E−06 | 2.54 | 1.41 | 3.67 | 1.16E−05 | 1.49E−03 |
| tumor necrosis factor- | | | | | | | | | | | | |
| related protein 1 | | | | | | | | | | | | |
| Lipopolysaccharide- | LBP | P18428 | 2 | 1.17 | 2.84 | 2.62E−06 | 6.35E−05 | 0.92 | 0.4 | 1.44 | 5.36E−04 | 2.12E−02 |
| binding protein | | | | | | | | | | | | |
| Beta-2-microglobulin | B2M | P61769 | 1.98 | 1.52 | 2.44 | 2.92E−17 | 9.07E−15 | 1.86 | 1.19 | 2.52 | 4.92E−08 | 4.27E−05 |
| ADAMTS-like | ADAMTSL2 | Q86TH1 | 1.97 | 1.53 | 2.41 | 1.99E−18 | 7.62E−16 | 1.32 | 0.56 | 2.08 | 6.99E−04 | 2.47E−02 |
| protein 2 | | | | | | | | | | | | |
| Tumor necrosis factor | TNFSF15 | 095150 | 1.85 | 1.29 | 2.42 | 1.35E−10 | 8.81E−09 | 2.42 | 1.46 | 3.37 | 6.96E−07 | 2.38E−04 |
| ligand superfamily | | | | | | | | | | | | |
| member 15 | | | | | | | | | | | | |
| CUB domain- | CDCP1 | Q9H5V8 | 1.79 | 1.5 | 2.07 | 9.65E−35 | 1.60E−31 | 1.68 | 1.22 | 2.15 | 1.59E−12 | 7.92E−09 |
| containing protein 1 | | | | | | | | | | | | |
| Cystatin-C | CST3 | P01034 | 1.78 | 1.24 | 2.33 | 1.88E−10 | 1.19E−08 | 1.83 | 0.94 | 2.71 | 5.67E−05 | 4.56E−03 |
| Triggering receptor | TREM1 | Q9NP99 | 1.76 | 1.33 | 2.19 | 9.42E−16 | 1.74E−13 | 1.39 | 0.78 | 2 | 7.93E−06 | 1.27E−03 |
| expressed on myeloid | | | | | | | | | | | | |
| cells 1 | | | | | | | | | | | | |
| Peptidyl-prolyl cis- | PPIC | P45877 | 1.73 | 1.17 | 2.3 | 1.93E−09 | 9.54E−08 | 1.53 | 0.66 | 2.39 | 5.54E−04 | 2.14E−02 |
| trans isomerase C | | | | | | | | | | | | |
| Gamma-glutamyl | GGH | Q92820 | 1.72 | 1.19 | 2.25 | 3.10E−10 | 1.86E−08 | 1.33 | 0.57 | 2.1 | 6.37E−04 | 2.34E−02 |
| hydrolase | | | | | | | | | | | | |
| C-C motif chemokine | CCL21 | O00585 | 1.68 | 1.25 | 2.1 | 2.05E−14 | 3.19E−12 | 1.57 | 0.91 | 2.23 | 2.89E−06 | 7.58E−04 |
| 21 | | | | | | | | | | | | |
| Sushi, von Willebrand | SVEP1 | Q4LDE5 | 1.63 | 1.25 | 2.01 | 7.30E−17 | 2.14E−14 | 1.82 | 1.16 | 2.48 | 6.00E−08 | 4.27E−05 |
| factor type A, EGF | | | | | | | | | | | | |
| and pentraxin domain- | | | | | | | | | | | | |
| containing protein | | | | | | | | | | | | |
| 1: EGF-like domains | | | | | | | | | | | | |
| 4-6 | | | | | | | | | | | | |
| Vesicular integral- | LMAN2 | Q12907 | 1.62 | 1.04 | 2.2 | 4.62E−08 | 1.70E−06 | 1.62 | 0.69 | 2.54 | 5.97E−04 | 2.22E−02 |
| membrane protein | | | | | | | | | | | | |
| VIP36 | | | | | | | | | | | | |
| Growth/differentiation | GDF15 | Q99988 | 1.61 | 1.31 | 1.91 | 1.28E−25 | 1.59E−22 | 0.88 | 0.37 | 1.38 | 6.48E−04 | 2.34E−02 |
| factor 15 | | | | | | | | | | | | |
| Retinoic acid receptor | RARRES2 | Q99969 | 1.59 | 1.03 | 2.15 | 2.96E−08 | 1.15E−06 | 1.31 | 0.49 | 2.13 | 1.82E−03 | 4.57E−02 |
| responder protein 2 | | | | | | | | | | | | |
| Axin-2 | AXIN2 | Q9Y2T1 | 1.53 | 1.02 | 2.03 | 3.23E−09 | 1.53E−07 | 0.86 | 0.44 | 1.29 | 6.26E−05 | 4.73E−03 |
| Sushi, von Willebrand | SVEP1 | Q4LDE5 | 1.5 | 1.11 | 1.89 | 8.08E−14 | 1.03E−11 | 1.61 | 0.88 | 2.33 | 1.35E−05 | 1.64E−03 |
| factor type A, EGF | | | | | | | | | | | | |
| and pentraxin domain- | | | | | | | | | | | | |
| containing protein | | | | | | | | | | | | |
| 1: Sushi 15-18 | | | | | | | | | | | | |
| Marginal zone B- and | MZB1 | Q8WU39 | 1.49 | 1.18 | 1.8 | 6.09E−21 | 3.37E−18 | 1.21 | 0.68 | 1.73 | 6.85E−06 | 1.26E−03 |
| B1-cell-specific | | | | | | | | | | | | |
| protein | | | | | | | | | | | | |
| Plexin-D1 | PLXND1 | Q9Y4D7 | 1.47 | 0.89 | 2.05 | 6.86E−07 | 1.91E−05 | 1.42 | 0.53 | 2.3 | 1.69E−03 | 4.39E−02 |
| Complement C2 | C2 | P06681 | 1.45 | 0.73 | 2.18 | 8.89E−05 | 1.29E−03 | 1.95 | 0.98 | 2.92 | 8.65E−05 | 6.06E−03 |
| Ganglioside GM2 | GM2A | P17900 | 1.45 | 0.97 | 1.94 | 4.58E−09 | 2.07E−07 | 1.59 | 0.79 | 2.39 | 1.01E−04 | 6.71E−03 |
| activator | | | | | | | | | | | | |
| Antileukoproteinase | SLPI | P03973 | 1.41 | 0.74 | 2.07 | 3.62E−05 | 5.99E−04 | 1.93 | 0.86 | 3 | 4.03E−04 | 1.79E−02 |
| E-selectin | SELE | P16581 | 1.4 | 1.05 | 1.75 | 4.24E−15 | 7.27E−13 | 0.94 | 0.43 | 1.44 | 2.90E−04 | 1.39E−02 |
| C-X-C motif | CXCL13 | O43927 | 1.35 | 1.03 | 1.68 | 5.15E−16 | 1.07E−13 | 1.37 | 0.76 | 1.98 | 1.21E−05 | 1.51E−03 |
| chemokine 13 | | | | | | | | | | | | |
| Tumor necrosis factor | TNFRSF1B | P20333 | 1.35 | 0.95 | 1.74 | 2.27E−11 | 1.74E−09 | 1.26 | 0.62 | 1.91 | 1.26E−04 | 7.74E−03 |
| receptor superfamily | | | | | | | | | | | | |
| member 1B | | | | | | | | | | | | |
| Thrombospondin-2 | THBS2 | P35442 | 1.34 | 1.08 | 1.61 | 1.08E−22 | 8.99E−20 | 0.88 | 0.39 | 1.37 | 4.57E−04 | 1.95E−02 |
| Tumor necrosis factor | TNFRSF17 | Q02223 | 1.31 | 0.85 | 1.78 | 3.07E−08 | 1.18E−06 | 1.17 | 0.46 | 1.88 | 1.21E−03 | 3.59E−02 |
| receptor superfamily | | | | | | | | | | | | |
| member 17 | | | | | | | | | | | | |
| Macrophage-capping | CAPG | P40121 | 1.29 | 0.91 | 1.67 | 2.15E−11 | 1.70E−09 | 1.71 | 1.16 | 2.25 | 8.15E−10 | 2.03E−06 |
| protein | | | | | | | | | | | | |
| Serine/threonine- | PPP4R3A | Q6IN85 | 1.29 | 0.91 | 1.67 | 3.49E−11 | 2.52E−09 | 1.27 | 0.64 | 1.9 | 8.79E−05 | 6.08E−03 |
| protein phosphatase 4 | | | | | | | | | | | | |
| regulatory subunit 3A | | | | | | | | | | | | |
| WAP four-disulfide | WFDC1 | Q9HC57 | 1.29 | 0.84 | 1.73 | 1.36E−08 | 5.66E−07 | 1.12 | 0.43 | 1.81 | 1.51E−03 | 4.09E−02 |
| core domain protein 1 | | | | | | | | | | | | |
| Neuroblastoma | NBL1 | P41271 | 1.28 | 0.91 | 1.66 | 1.93E−11 | 1.57E−09 | 1.57 | 0.87 | 2.27 | 1.09E−05 | 1.44E−03 |
| suppressor of | | | | | | | | | | | | |
| tumorigenicity 1 | | | | | | | | | | | | |
| Golgi membrane | GOLM1 | Q8NBJ4 | 1.25 | 0.9 | 1.59 | 1.28E−12 | 1.32E−10 | 1.05 | 0.49 | 1.61 | 2.28E−04 | 1.17E−02 |
| protein 1 | | | | | | | | | | | | |
| Thioredoxin domain- | TXNDC5 | Q8NBS9 | 1.23 | 0.82 | 1.65 | 4.35E−09 | 2.01E−07 | 1.44 | 0.63 | 2.26 | 5.26E−04 | 2.11E−02 |
| containing protein 5 | | | | | | | | | | | | |
| C-C motif chemokine 3 | CCL3 | P10147 | 1.23 | 0.83 | 1.63 | 2.02E−09 | 9.87E−08 | 0.85 | 0.34 | 1.37 | 1.23E−03 | 3.59E−02 |
| Matrix Gla protein | MGP | P08493 | 1.21 | 0.63 | 1.79 | 4.54E−05 | 7.20E−04 | 2.05 | 1.03 | 3.08 | 8.55E−05 | 6.06E−03 |
| Calsyntenin-3 | CLSTN3 | Q9BQT9 | 1.21 | 0.82 | 1.61 | 1.94E−09 | 9.54E−08 | 1.49 | 0.92 | 2.07 | 3.55E−07 | 1.61E−04 |
| Ephrin type-A | EPHA2 | P29317 | 1.21 | 0.75 | 1.66 | 2.02E−07 | 6.24E−06 | 1.35 | 0.57 | 2.12 | 6.46E−04 | 2.34E−02 |
| receptor 2 | | | | | | | | | | | | |
| Leucine-rich repeats | LRIG1 | Q96JA1 | 1.19 | 0.61 | 1.78 | 6.52E−05 | 9.87E−04 | 1.73 | 0.88 | 2.58 | 6.58E−05 | 4.89E−03 |
| and immunoglobulin- | | | | | | | | | | | | |
| like domains protein 1 | | | | | | | | | | | | |
| Low-density | LRP11 | Q86VZ4 | 1.13 | 0.51 | 1.75 | 3.83E−04 | 4.56E−03 | 1.74 | 0.87 | 2.62 | 1.00E−04 | 6.71E−03 |
| lipoprotein receptor- | | | | | | | | | | | | |
| related protein 11 | | | | | | | | | | | | |
| IGF-like family | IGFLR1 | Q9H665 | 1.13 | 0.8 | 1.47 | 2.81E−11 | 2.12E−09 | 1.08 | 0.57 | 1.59 | 3.31E−05 | 3.17E−03 |
| receptor 1 | | | | | | | | | | | | |
| Ribonuclease K6 | RNASE6 | Q93091 | 1.12 | 0.75 | 1.49 | 4.19E−09 | 1.95E−07 | 1.36 | 0.71 | 2 | 3.64E−05 | 3.42E−03 |
| Interleukin enhancer- | ILF3 | Q12906 | 1.09 | 0.53 | 1.64 | 1.22E−04 | 1.71E−03 | 1.6 | 0.76 | 2.44 | 1.98E−04 | 1.07E−02 |
| binding factor 3 | | | | | | | | | | | | |
| Macrophage | MMP12 | P39900 | 1.06 | 0.76 | 1.35 | 2.70E−12 | 2.63E−10 | 1.11 | 0.62 | 1.6 | 1.02E−05 | 1.44E−03 |
| metalloelastase | | | | | | | | | | | | |
| Kallikrein-10 | KLK10 | O43240 | 1.04 | 0.66 | 1.42 | 1.16E−07 | 3.92E−06 | 1.09 | 0.47 | 1.71 | 5.58E−04 | 2.14E−02 |
| Elafin | PI3 | P19957 | 1.04 | 0.72 | 1.36 | 1.66E−10 | 1.06E−08 | 0.96 | 0.48 | 1.44 | 1.02E−04 | 6.71E−03 |
| BPI fold-containing | BPIFB1 | Q8TDL5 | 1.04 | 0.76 | 1.32 | 7.54E−13 | 8.73E−11 | 0.66 | 0.29 | 1.04 | 5.66E−04 | 2.14E−02 |
| family B member 1 | | | | | | | | | | | | |
| Erythropoietin | EPO | P01588 | 1.03 | 0.7 | 1.35 | 9.87E−10 | 5.46E−08 | 1.02 | 0.57 | 1.46 | 7.18E−06 | 1.26E−03 |
| Insulin-like growth | IGFBP4 | P22692 | 1.02 | 0.41 | 1.63 | 1.13E−03 | 1.05E−02 | 2.54 | 1.62 | 3.46 | 5.89E−08 | 4.27E−05 |
| factor-binding protein 4 | | | | | | | | | | | | |
| Fibulin-5 | FBLN5 | Q9UBX5 | 1.02 | 0.52 | 1.51 | 5.32E−05 | 8.22E−04 | 2.28 | 1.2 | 3.37 | 3.87E−05 | 3.44E−03 |
| Intercellular adhesion | ICAM1 | P05362 | 1 | 0.66 | 1.34 | 7.68E−09 | 3.27E−07 | 1.08 | 0.55 | 1.6 | 6.26E−05 | 4.73E−03 |
| molecule 1 | | | | | | | | | | | | |
| Transmembrane | TMED10 | P49755 | 0.98 | 0.55 | 1.4 | 6.27E−06 | 1.35E−04 | 1.22 | 0.54 | 1.9 | 4.77E−04 | 2.00E−02 |
| emp24 domain- | | | | | | | | | | | | |
| containing protein 10 | | | | | | | | | | | | |
| Serpin B8 | SERPINB8 | P50452 | 0.97 | 0.42 | 1.52 | 5.73E−04 | 6.26E−03 | 1.57 | 0.75 | 2.39 | 1.75E−04 | 9.84E−03 |
| Protein CYR61 | CCN1 | O00622 | 0.97 | 0.53 | 1.41 | 1.65E−05 | 3.06E−04 | 1.15 | 0.55 | 1.76 | 1.89E−04 | 1.04E−02 |
| B melanoma antigen 2 | BAGE2 | Q86Y30 | 0.96 | 0.53 | 1.39 | 1.09E−05 | 2.17E−04 | 1.99 | 1.21 | 2.77 | 6.37E−07 | 2.38E−04 |
| Phospholipase A2, | PLA2G2A | P14555 | 0.96 | 0.65 | 1.27 | 1.53E−09 | 7.96E−08 | 0.9 | 0.4 | 1.41 | 4.84E−04 | 2.00E−02 |
| membrane associated | | | | | | | | | | | | |
| Thrombospondin-3 | THBS3 | P49746 | 0.95 | 0.5 | 1.4 | 3.77E−05 | 6.19E−04 | 1.21 | 0.54 | 1.88 | 4.02E−04 | 1.79E−02 |
| C-C motif chemokine | CCL22 | O00626 | 0.95 | 0.6 | 1.29 | 6.25E−08 | 2.21E−06 | 1.17 | 0.55 | 1.79 | 2.18E−04 | 1.15E−02 |
| 22 | | | | | | | | | | | | |
| Cadherin- | CDH11 | P55287 | 0.94 | 0.73 | 1.15 | 5.12E−18 | 1.82E−15 | 0.7 | 0.43 | 0.98 | 7.17E−07 | 2.38E−04 |
| 11: Extracellular | | | | | | | | | | | | |
| domain | | | | | | | | | | | | |
| Alpha-1- | SERPINA3 | P01011 | 0.88 | 0.45 | 1.3 | 4.51E−05 | 7.19E−04 | 1.35 | 0.66 | 2.05 | 1.44E−04 | 8.63E−03 |
| antichymotrypsin | | | | | | | | | | | | |
| complex | | | | | | | | | | | | |
| Troponin T, cardiac | TNNT2 | P45379 | 0.86 | 0.49 | 1.24 | 6.65E−06 | 1.42E−04 | 1.11 | 0.61 | 1.61 | 1.62E−05 | 1.92E−03 |
| muscle | | | | | | | | | | | | |
| Lysozyme C | LYZ | P61626 | 0.86 | 0.45 | 1.26 | 3.30E−05 | 5.54E−04 | 1 | 0.41 | 1.59 | 9.29E−04 | 2.91E−02 |
| SLAM family | SLAMF7 | Q9NQ25 | 0.86 | 0.61 | 1.11 | 2.89E−11 | 2.15E−09 | 0.67 | 0.3 | 1.04 | 4.50E−04 | 1.93E−02 |
| member 7 | | | | | | | | | | | | |
| Fibroblast growth | FGF23 | Q9GZV9 | 0.85 | 0.52 | 1.18 | 4.14E−07 | 1.20E−05 | 0.84 | 0.41 | 1.27 | 1.28E−04 | 7.77E−03 |
| factor 23 | | | | | | | | | | | | |
| Trafficking protein | TRAPPC3 | O43617 | 0.83 | 0.45 | 1.21 | 1.79E−05 | 3.28E−04 | 1.16 | 0.45 | 1.88 | 1.40E−03 | 3.94E−02 |
| particle complex | | | | | | | | | | | | |
| subunit 3 | | | | | | | | | | | | |
| Oncoprotein-induced | OIT3 | Q8WWZ8 | 0.8 | 0.38 | 1.22 | 1.79E−04 | 2.41E−03 | 1.37 | 0.67 | 2.07 | 1.17E−04 | 7.35E−03 |
| transcript 3 protein | | | | | | | | | | | | |
| Cerebellin-4 | CBLN4 | Q9NTU7 | 0.8 | 0.32 | 1.29 | 1.08E−03 | 1.02E−02 | −1.52 | −2.19 | −0.85 | 9.16E−06 | 1.42E−03 |
| Nidogen-1 | NID1 | P14543 | 0.79 | 0.3 | 1.28 | 1.54E−03 | 1.34E−02 | 1.86 | 0.99 | 2.74 | 2.92E−05 | 2.96E−03 |
| C-X-C motif | CXCL10 | P02778 | 0.79 | 0.47 | 1.1 | 9.94E−07 | 2.59E−05 | 1.08 | 0.6 | 1.56 | 1.00E−05 | 1.44E−03 |
| chemokine 10 | | | | | | | | | | | | |
| Protein ABHD14A | ABHD14A | Q9BUJ0 | 0.77 | 0.39 | 1.15 | 7.52E−05 | 1.11E−03 | 1.65 | 0.95 | 2.34 | 3.60E−06 | 8.53E−04 |
| Ribonuclease | RNASE1 | P07998 | 0.74 | 0.49 | 0.99 | 6.29E−09 | 2.72E−07 | 0.78 | 0.36 | 1.2 | 2.50E−04 | 1.24E−02 |
| pancreatic | | | | | | | | | | | | |
| Vascular endothelial | VEGFD | O43915 | 0.71 | 0.37 | 1.06 | 5.78E−05 | 8.88E−04 | 0.87 | 0.42 | 1.32 | 1.63E−04 | 9.41E−03 |
| growth factor D | | | | | | | | | | | | |
| Serum amyloid A-2 | SAA2 | P0DJI9 | 0.71 | 0.5 | 0.93 | 1.32E−10 | 8.75E−09 | 0.63 | 0.28 | 0.99 | 4.87E−04 | 2.00E−02 |
| protein | | | | | | | | | | | | |
| RNA-binding protein | EWSR1 | Q01844 | 0.7 | 0.24 | 1.17 | 3.23E−03 | 2.38E−02 | 1.79 | 0.85 | 2.72 | 1.76E−04 | 9.84E−03 |
| EWS | | | | | | | | | | | | |
| C-reactive protein | CRP | P02741 | 0.66 | 0.49 | 0.82 | 3.80E−14 | 5.25E−12 | 0.4 | 0.16 | 0.65 | 1.33E−03 | 3.81E−02 |
| Thiosulfate | TST | Q16762 | 0.64 | 0.37 | 0.91 | 3.24E−06 | 7.75E−05 | 0.7 | 0.29 | 1.11 | 8.90E−04 | 2.89E−02 |
| sulfurtransferase | | | | | | | | | | | | |
| Syntaxin-12 | STX12 | Q86Y82 | 0.63 | 0.29 | 0.97 | 2.52E−04 | 3.23E−03 | 0.95 | 0.41 | 1.49 | 5.57E−04 | 2.14E−02 |
| Ubiquitin-like protein | ISG15 | P05161 | 0.61 | 0.36 | 0.86 | 2.77E−06 | 6.70E−05 | 0.89 | 0.5 | 1.28 | 6.53E−06 | 1.25E−03 |
| ISG15 | | | | | | | | | | | | |
| Lamina-associated | TMPO | P42167 | 0.6 | 0.25 | 0.95 | 6.95E−04 | 7.15E−03 | 1.62 | 1 | 2.23 | 2.54E−07 | 1.40E−04 |
| polypeptide 2, | | | | | | | | | | | | |
| isoforms beta/gamma | | | | | | | | | | | | |
| DnaJ homolog | DNAJB12 | Q9NXW2 | 0.55 | 0.25 | 0.85 | 2.77E−04 | 3.48E−03 | 1.15 | 0.48 | 1.82 | 7.71E−04 | 2.66E−02 |
| subfamily B member | | | | | | | | | | | | |
| 12 | | | | | | | | | | | | |
| Gastrokine-2 | GKN2 | Q86XP6 | 0.55 | 0.26 | 0.84 | 2.18E−04 | 2.83E−03 | 0.68 | 0.28 | 1.08 | 8.97E−04 | 2.89E−02 |
| C-X-C motif | CXCL11 | O14625 | 0.55 | 0.35 | 0.76 | 1.51E−07 | 4.95E−06 | 0.48 | 0.2 | 0.77 | 8.82E−04 | 2.89E−02 |
| chemokine 11 | | | | | | | | | | | | |
| Lactoperoxidase | LPO | P22079 | 0.54 | 0.22 | 0.86 | 1.07E−03 | 1.01E−02 | 0.88 | 0.46 | 1.29 | 3.14E−05 | 3.12E−03 |
| Thioredoxin reductase | TXNRD1 | Q16881 | 0.53 | 0.23 | 0.83 | 5.25E−04 | 5.88E−03 | 0.99 | 0.45 | 1.54 | 3.68E−04 | 1.67E−02 |
| 1, cytoplasmic | | | | | | | | | | | | |
| Troponin I, fast | TNNI2 | P48788 | 0.52 | 0.29 | 0.75 | 7.67E−06 | 1.59E−04 | 0.57 | 0.23 | 0.91 | 9.78E−04 | 2.99E−02 |
| skeletal muscle | | | | | | | | | | | | |
| Gamma-interferon- | IFI16 | Q16666 | 0.45 | 0.18 | 0.72 | 9.26E−04 | 9.01E−03 | 0.97 | 0.45 | 1.49 | 2.46E−04 | 1.24E−02 |
| inducible protein | | | | | | | | | | | | |
| 16: Isoform 2, | | | | | | | | | | | | |
| Hematopoietic | | | | | | | | | | | | |
| expression, interferon- | | | | | | | | | | | | |
| inducible nature, and | | | | | | | | | | | | |
| nuclear localization 1 | | | | | | | | | | | | |
| Granulocyte colony- | CSF3 | P09919 | 0.45 | 0.17 | 0.74 | 1.69E−03 | 1.46E−02 | 0.87 | 0.45 | 1.28 | 5.01E−05 | 4.15E−03 |
| stimulating factor | | | | | | | | | | | | |
| C-C motif chemokine | CCL19 | Q99731 | 0.45 | 0.15 | 0.75 | 3.34E−03 | 2.45E−02 | 0.86 | 0.37 | 1.35 | 5.63E−04 | 2.14E−02 |
| 19 | | | | | | | | | | | | |
| Tapasin | TAPBP | O15533 | 0.45 | 0.17 | 0.73 | 1.48E−03 | 1.30E−02 | 0.61 | 0.28 | 0.94 | 3.40E−04 | 1.56E−02 |
| Chitotriosidase-1 | CHIT1 | Q13231 | 0.44 | 0.23 | 0.65 | 4.20E−05 | 6.81E−04 | 0.63 | 0.28 | 0.98 | 4.24E−04 | 1.84E−02 |
| Interferon-induced | MX1 | P20591 | 0.43 | 0.17 | 0.69 | 1.34E−03 | 1.20E−02 | 0.89 | 0.44 | 1.34 | 1.11E−04 | 7.06E−03 |
| GTP-binding protein | | | | | | | | | | | | |
| Mx1 | | | | | | | | | | | | |
| Beta-defensin 4A | DEFB4A | O15263 | 0.37 | 0.23 | 0.52 | 3.49E−07 | 1.03E−05 | 0.52 | 0.31 | 0.74 | 2.32E−06 | 6.43E−04 |
| Chitinase-3-like | CHI3L1 | P36222 | 0.22 | 0.08 | 0.35 | 1.47E−03 | 1.30E−02 | 0.36 | 0.15 | 0.58 | 9.21E−04 | 2.90E−02 |
| protein 1 | | | | | | | | | | | | |
| Zymogen granule | ZG16 | O60844 | 0.22 | 0.16 | 0.29 | 1.21E−10 | 8.16E−09 | 0.2 | 0.12 | 0.27 | 1.44E−07 | 8.96E−05 |
| membrane protein 16 | | | | | | | | | | | | |
| Protein kinase C- | NELL1 | Q92832 | −0.55 | −0.88 | −0.22 | 1.14E−03 | 1.06E−02 | −0.88 | −1.43 | −0.33 | 1.82E−03 | 4.57E−02 |
| binding protein | | | | | | | | | | | | |
| NELL1 | | | | | | | | | | | | |
| Advanced | AGER | Q15109 | −0.56 | −0.78 | −0.35 | 3.02E−07 | 9.06E−06 | −0.45 | −0.73 | −0.17 | 1.68E−03 | 4.39E−02 |
| glycosylation end | | | | | | | | | | | | |
| product-specific | | | | | | | | | | | | |
| receptor, soluble | | | | | | | | | | | | |
| Cartilage intermediate | CILP2 | Q8IUL8 | −0.57 | −0.98 | −0.16 | 6.15E−03 | 3.92E−02 | −1.34 | −2.03 | −0.66 | 1.24E−04 | 7.74E−03 |
| layer protein 2 | | | | | | | | | | | | |
| Glypican-3 | GPC3 | P51654 | −0.59 | −0.98 | −0.2 | 2.78E−03 | 2.13E−02 | −0.95 | −1.49 | −0.41 | 5.41E−04 | 2.12E−02 |
| N-acetylglucosamine- | NAGPA | Q9UK23 | −0.78 | −1.3 | −0.26 | 3.02E−03 | 2.26E−02 | −1.53 | −2.48 | −0.59 | 1.47E−03 | 4.00E−02 |
| 1-phosphodiester | | | | | | | | | | | | |
| alpha-N- | | | | | | | | | | | | |
| acetylglucosaminidase | | | | | | | | | | | | |
| Beta-1,4- | B4GALT6 | Q9UBX8 | −0.82 | −1.34 | −0.3 | 1.93E−03 | 1.62E−02 | −1.56 | −2.38 | −0.74 | 2.05E−04 | 1.10E−02 |
| galactosyltransferase 6 | | | | | | | | | | | | |
| Prostaglandin F2 | PTGFRN | Q9P2B2 | −0.83 | −1.32 | −0.34 | 9.44E−04 | 9.15E−03 | −1.38 | −2.1 | −0.67 | 1.59E−04 | 9.34E−03 |
| receptor negative | | | | | | | | | | | | |
| regulator | | | | | | | | | | | | |
| Voltage-dependent | CACNA2D3 | Q8IZS8 | −0.93 | −1.44 | −0.42 | 3.48E−04 | 4.23E−03 | −1.6 | −2.33 | −0.87 | 1.76E−05 | 2.04E−03 |
| calcium channel | | | | | | | | | | | | |
| subunit alpha-2/delta-3 | | | | | | | | | | | | |
| Interleukin-1 receptor | IL1R2 | P27930 | −0.98 | −1.55 | −0.41 | 7.76E−04 | 7.83E−03 | −1.8 | −2.64 | −0.97 | 2.31E−05 | 2.62E−03 |
| type 2 | | | | | | | | | | | | |
| Golgi-associated plant | GLIPR2 | Q9H4G4 | −1 | −1.6 | −0.4 | 1.10E−03 | 1.03E−02 | −1.91 | −2.9 | −0.92 | 1.57E−04 | 9.32E−03 |
| pathogenesis-related | | | | | | | | | | | | |
| protein 1 | | | | | | | | | | | | |
| Anthrax toxin | ANTXR1 | Q9H6X2 | −1.14 | −1.68 | −0.59 | 4.47E−05 | 7.15E−04 | −1.52 | −2.26 | −0.78 | 5.89E−05 | 4.66E−03 |
| receptor 1 | | | | | | | | | | | | |
| Neurocan core protein | NCAN | O14594 | −1.2 | −1.6 | −0.8 | 4.54E−09 | 2.07E−07 | −1.32 | −1.98 | −0.66 | 9.45E−05 | 6.44E−03 |
| Contactin-4 | CNTN4 | Q8IWV2 | −1.34 | −2.07 | −0.61 | 3.11E−04 | 3.85E−03 | −2.29 | −3.31 | −1.27 | 1.10E−05 | 1.44E−03 |
| Semaphorin-3G | SEMA3G | Q9NS98 | −1.37 | −2.05 | −0.69 | 7.76E−05 | 1.14E−03 | −2.24 | −3.21 | −1.26 | 7.31E−06 | 1.26E−03 |
| Thrombin | F2 | P00734 | −1.39 | −1.75 | −1.03 | 5.30E−14 | 7.13E−12 | −1.54 | −2.29 | −0.79 | 6.02E−05 | 4.68E−03 |
| Leucine-rich repeats | LRIG3 | Q6UXM1 | −1.39 | −1.95 | −0.83 | 1.27E−06 | 3.28E−05 | −1.79 | −2.81 | −0.78 | 5.12E−04 | 2.07E−02 |
| and immunoglobulin- | | | | | | | | | | | | |
| like domains protein 3 | | | | | | | | | | | | |
| Interleukin-6 receptor | IL6R | P08887 | −1.42 | −1.92 | −0.91 | 4.48E−08 | 1.66E−06 | −1.45 | −2.17 | −0.73 | 8.07E−05 | 5.82E−03 |
| subunit alpha | | | | | | | | | | | | |
| A disintegrin and | ADAMTS13 | Q76LX8 | −1.58 | −2.08 | −1.08 | 4.71E−10 | 2.79E−08 | −1.32 | −2.05 | −0.6 | 3.19E−04 | 1.50E−02 |
| metalloproteinase | | | | | | | | | | | | |
| with thrombospondin | | | | | | | | | | | | |
| motifs 13 | | | | | | | | | | | | |
| Cholinesterase | BCHE | P06276 | −1.62 | −2.21 | −1.04 | 5.93E−08 | 2.14E−06 | −1.5 | −2.39 | −0.61 | 9.37E−04 | 2.91E−02 |
| Sodium/potassium- | ATP1B1 | P05026 | −1.63 | −2.02 | −1.23 | 8.06E−16 | 1.57E−13 | −1.17 | −1.69 | −0.65 | 1.04E−05 | 1.44E−03 |
| transporting ATPase | | | | | | | | | | | | |
| subunit beta-1 | | | | | | | | | | | | |
| Alpha-2-HS- | AHSG | P02765 | −1.64 | −2.47 | −0.81 | 1.08E−04 | 1.54E−03 | −3.14 | −4.65 | −1.64 | 4.36E−05 | 3.81E−03 |
| glycoprotein | | | | | | | | | | | | |
| Collagen alpha- | COL13A1 | Q5TAT6 | −1.89 | −2.52 | −1.26 | 5.36E−09 | 2.36E−07 | −2.44 | −3.49 | −1.4 | 4.53E−06 | 9.14E−04 |
| 1(XIII) chain | | | | | | | | | | | | |
| Afamin | AFM | P43652 | −2.06 | −2.78 | −1.34 | 2.50E−08 | 9.79E−07 | −2.65 | −3.77 | −1.52 | 4.21E−06 | 9.11E−04 |
| Receptor-type | PTPRJ | Q12913 | −2.1 | −2.8 | −1.4 | 5.18E−09 | 2.32E−07 | −1.76 | −2.78 | −0.75 | 6.75E−04 | 2.41E−02 |
| tyrosine-protein | | | | | | | | | | | | |
| phosphatase eta | | | | | | | | | | | | |
| Coiled-coil domain- | CCDC126 | Q96EE4 | −2.29 | −2.83 | −1.75 | 1.60E−16 | 3.80E−14 | −1.28 | −2.02 | −0.55 | 6.47E−04 | 2.34E−02 |
| containing protein 126 | | | | | | | | | | | | |
| Ciliary neurotrophic | CNTFR | P26992 | −2.32 | −3 | −1.63 | 3.49E−11 | 2.52E−09 | −1.78 | −2.74 | −0.83 | 2.53E−04 | 1.25E−02 |
| factor receptor subunit | | | | | | | | | | | | |
| alpha | | | | | | | | | | | | |
| Group XIIB secretory | PLA2G12B | Q9BX93 | −2.33 | −2.82 | −1.84 | 2.89E−20 | 1.20E−17 | −1.2 | −1.89 | −0.5 | 8.03E−04 | 2.72E−02 |
| phospholipase A2-like | | | | | | | | | | | | |
| protein | | | | | | | | | | | | |
| Contactin-1 | CNTN1 | Q12860 | −2.37 | −3.12 | −1.61 | 7.59E−10 | 4.37E−08 | −1.84 | −2.91 | −0.77 | 7.79E−04 | 2.67E−02 |
| Matrix-remodeling- | MXRA8 | Q9BRK3 | −2.39 | −3.04 | −1.74 | 7.71E−13 | 8.73E−11 | −2.09 | −3 | −1.17 | 7.92E−06 | 1.27E−03 |
| associated protein | | | | | | | | | | | | |
| 8: Extracellular | | | | | | | | | | | | |
| domain | | | | | | | | | | | | |
| Heparan-sulfate 6-O- | HS6ST3 | Q8IZP7 | −2.47 | −3.4 | −1.55 | 1.69E−07 | 5.37E−06 | −1.96 | −3.12 | −0.8 | 9.07E−04 | 2.89E−02 |
| sulfotransferase 3 | | | | | | | | | | | | |
| B melanoma antigen 3 | BAGE3 | Q86Y29 | −2.76 | −3.56 | −1.95 | 1.98E−11 | 1.59E−09 | −2.22 | −3.39 | −1.06 | 1.93E−04 | 1.06E−02 |
| Vitamin K-dependent | PROC | P04070 | −2.8 | −3.52 | −2.08 | 3.29E−14 | 4.81E−12 | −2.18 | −3.4 | −0.96 | 4.80E−04 | 2.00E−02 |
| protein C | | | | | | | | | | | | |
| Apolipoprotein M | APOM | O95445 | −2.86 | −3.43 | −2.28 | 2.36E−22 | 1.68E−19 | −1.49 | −2.42 | −0.56 | 1.72E−03 | 4.43E−02 |
| Neuropeptide S | NPS | P0C0P6 | −3.28 | −4.12 | −2.43 | 3.25E−14 | 4.81E−12 | −2.56 | −3.92 | −1.19 | 2.44E−04 | 1.24E−02 |
| Epidermal growth | EGFR | P00533 | −4.36 | −5.27 | −3.44 | 1.17E−20 | 5.29E−18 | −3.58 | −4.98 | −2.19 | 5.23E−07 | 2.17E−04 |
| factor receptor | | | | | | | | | | | | |

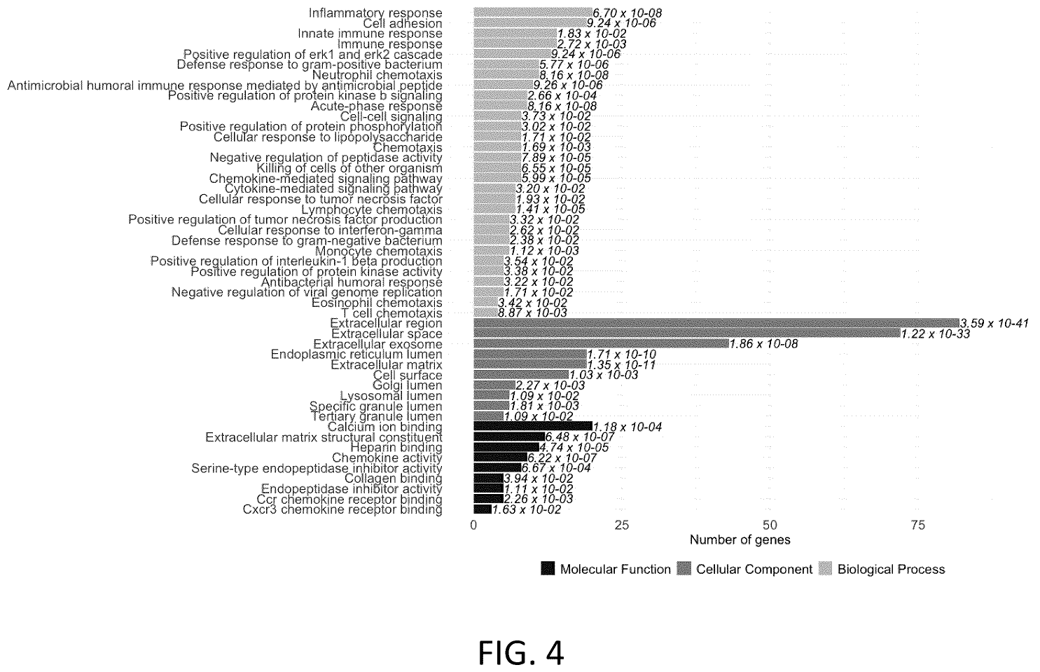

Pathway enrichment analysis showed overrepresentation of proteins involved in multiple pathways including GO Biological Processes in inflammatory response, cell adhesion, immune response, ERK1/2 regulation, signaling; GO Cellular Components in extracellular regions, spaces, exosome, and matrix; and GO Molecular Functions including calcium ion and heparin binding (FIG. 4, Table 8).

TABLE 8
Pathway enrichment analysis of proteins associated with QIA in COPDGene and CARDIA

| Gene | Gene | | | | |
|---|---|---|---|---|---|
| ontology | ontology | | Gene | | FDR |
| category | term name | Term description | count | Gene list (Entreze GeneIDs) | p-value |
| Molecular | GO:0008009 | Chemokine activity | 9 | 6373, 6362, 6363, 58191, 6366, 6367, 6348, 10563, 3627 | 6.22E−07 |
| Function | | | | | |
| Molecular | GO:0005201 | Extracellular matrix | 12 | 1305, 1116, 7059, 7058, 10418, 2202, 4256, 170392, 4811, | 6.48E−07 |
| Function | | structural constituent | | 3491, 4060, 340267 | |
| Molecular | GO:0008201 | Heparin binding | 11 | 6373, 1305, 7059, 6288, 7058, 2147, 10563, 4745, 8828, | 4.7421E−05 |
| Function | | | | 3491, 3627 | |
| Molecular | GO:0005509 | Calcium ion binding | 20 | 11093, 5320, 7059, 7058, 4321, 4256, 84647, 10516, 1401, | 0.000117933 |
| Function | | | | 2147, 2202, 79987, 1463, 822, 170392, 9746, 5624, 4745, | |
| | | | | 4811, 1009 | |
| Molecular | GO:0004867 | Serine-type | 8 | 12, 10406, 5266, 58189, 1293, 340267, 5271, 6590 | 0.000666971 |
| Function | | endopeptidase inhibitor | | | |
| | | activity | | | |
| Molecular | GO:0048020 | Ccr chemokine receptor | 5 | 6362, 6363, 6366, 6367, 6348 | 0.002263213 |
| Function | | binding | | | |
| Molecular | GO:0004866 | Endopeptidase inhibitor | 5 | 10406, 5266, 1471, 197, 6590 | 0.011132722 |
| Function | | activity | | | |
| Molecular | GO:0048248 | Cxcr3 chemokine | 3 | 6373, 10563, 3627 | 0.016257438 |
| Function | | receptor binding | | | |
| Molecular | GO:0005518 | Collagen binding | 5 | 4321, 114897, 84168, 4811, 4060 | 0.039430012 |
| Function | | | | | |
| Cellular | GO:0005576 | Extracellular region | 82 | 58191, 5266, 4256, 590, 6590, 197, 84647, 2147, 6348, | 3.59E−41 |
| Component | | | | 5655, 152330, 717, 12, 6363, 7133, 7059, 6366, 6288, | |
| | | | | 7058, 7850, 4025, 6367, 4069, 2760, 1471, 114897, 3570, | |
| | | | | 3491, 4060, 1305, 81567, 3929, 567, 3487, 1463, 2277, | |
| | | | | 6039, 140689, 3609, 5624, 4811, 55937, 6373, 5320, 2056, | |
| | | | | 1440, 173, 8074, 85319, 1118, 177, 54210, 10516, 1116, | |
| | | | | 85318, 5919, 2202, 9518, 9636, 4745, 9719, 8828, 340267, | |
| | | | | 90693, 11093, 594857, 4321, 148113, 64866, 1293, | |
| | | | | 353514, 10406, 1401, 152007, 79987, 8836, 10563, 9966, | |
| | | | | 51237, 92747, 629, 3627 | |
| Cellular | GO:0005615 | Extracellular space | 72 | 58191, 5266, 200504, 590, 6590, 197, 51280, 10418, 2147, | 1.22E−33 |
| Component | | | | 6348, 717, 6362, 12, 6363, 6366, 6401, 4025, 6367, 4069, | |
| | | | | 1471, 114897, 3570, 4060, 5271, 1305, 121227, 3929, 567, | |
| | | | | 3487, 2277, 6039, 170392, 140689, 5624, 6373, 7263, | |
| | | | | 5320, 653808, 2056, 1440, 26018, 3383, 173, 8074, 1118, | |
| | | | | 10516, 1116, 5919, 2202, 9518, 1956, 340267, 11093, | |
| | | | | 100532736, 4321, 148113, 58189, 1293, 353514, 10406, | |
| | | | | 56920, 1401, 152007, 79987, 822, 8836, 10563, 9966, | |
| | | | | 92747, 10960, 629, 3627 | |
| Cellular | GO:0031012 | Extracellular matrix | 19 | 11093, 5266, 4321, 4256, 26018, 1293, 3491, 4060, 197, | 1.35E−11 |
| Component | | | | 1305, 121227, 10516, 1116, 5919, 10418, 2202, 1463, | |
| | | | | 9719, 340267 | |
| Cellular | GO:0005788 | Endoplasmic reticulum | 19 | 11093, 1471, 1293, 590, 3491, 197, 8074, 51280, 1305, | 1.71E−10 |
| Component | | lumen | | 81567, 2147, 10418, 567, 54587, 3487, 2719, 51237, 5624, | |
| | | | | 340267 | |
| Cellular | GO:0070062 | Extracellular exosome | 43 | 5320, 6035, 4256, 3383, 173, 197, 5480, 6590, 10516, | 1.86E−08 |
| Component | | | | 1116, 2202, 2147, 9518, 717, 7296, 12, 6289, 6288, 5795, | |
| | | | | 4069, 4025, 2760, 148113, 1471, 1272, 1293, 481, 4060, | |
| | | | | 5271, 81567, 56920, 10406, 3929, 152007, 567, 54587, | |
| | | | | 822, 8836, 92747, 4811, 10960, 629, 1009 | |
| Cellular | GO:0009986 | Cell surface | 16 | 11093, 5795, 2056, 3383, 353514, 177, 3929, 54587, | 0.001030241 |
| Component | | | | 170392, 84168, 9746, 2719, 1969, 10960, 1956, 5738 | |
| Cellular | GO:0035580 | Specific granule lumen | 6 | 1118, 1116, 567, 4069, 8836, 6590 | 0.001810878 |
| Component | | | | | |
| Cellular | GO:0005796 | Golgi lumen | 7 | 653808, 2147, 1463, 2719, 5624, 4060, 8074 | 0.002269343 |
| Component | | | | | |
| Cellular | GO:1904724 | Tertiary granule lumen | 5 | 1118, 567, 4069, 8836, 1471 | 0.010925199 |
| Component | | | | | |
| Cellular | GO:0043202 | Lysosomal lumen | 6 | 81567, 1463, 1440, 2760, 2719, 4060 | 0.010925199 |
| Component | | | | | |
| Biological | GO:0006954 | Inflammatory response | 20 | 6373, 6362, 12, 6363, 7133, 5320, 6366, 6401, 4069, 6367, | 6.70E−08 |
| Process | | | | 3428, 177, 1116, 1401, 5919, 3487, 6348, 10563, 1969, | |
| | | | | 3627 | |
| Biological | GO:0006953 | Acute-phase response | 9 | 12, 1401, 6289, 6288, 2147, 3929, 2056, 3570, 197 | 8.16E−08 |
| Process | | | | | |
| Biological | GO:0030593 | Neutrophil chemotaxis | 11 | 6373, 6362, 54210, 6363, 6366, 6288, 3929, 6367, 6348, | 8.16E−08 |
| Process | | | | 10563, 3627 | |
| Biological | GO:0050830 | Defense response to | 11 | 1401, 5320, 653808, 5919, 6035, 3929, 567, 4069, 6039, | 5.77E−06 |
| Process | | gram-positive bacterium | | 1969, 3570 | |
| Biological | GO:0007155 | Cell adhesion | 19 | 7059, 7058, 6401, 23114, 1272, 1293, 3383, 481, 3491, | 9.24E−06 |
| Process | | | | 57823, 10418, 54587, 79987, 1463, 1969, 8828, 152330, | |
| | | | | 340267, 1009 | |
| Biological | GO:0070374 | Positive regulation of | 13 | 6362, 6363, 5320, 6366, 6367, 2056, 3383, 8074, 177, | 9.24E−06 |
| Process | | erk1 and erk2 cascade | | 1116, 152007, 6348, 1956 | |
| Biological | GO:0061844 | Antimicrobial humoral | 10 | 6373, 6362, 6363, 6366, 2147, 567, 6367, 6039, 10563, | 9.26E−06 |
| Process | | immune response | | 3627 | |
| | | mediated by | | | |
| | | antimicrobial peptide | | | |
| Biological | GO:0048247 | Lymphocyte chemotaxis | 7 | 6362, 6363, 58191, 6366, 6288, 6367, 6348 | 1.40908E−05 |
| Process | | | | | |
| Biological | GO:0070098 | Chemokine-mediated | 8 | 6373, 6362, 6363, 6366, 6367, 6348, 10563, 3627 | 5.98897E−05 |
| Process | | signaling pathway | | | |
| Biological | GO:0031640 | Killing of cells of other | 8 | 6373, 6362, 6363, 6366, 4069, 6367, 10563, 3627 | 6.54854E−05 |
| Process | | organism | | | |
| Biological | GO:0010466 | Negative regulation of | 8 | 10406, 5266, 58189, 2719, 1471, 1293, 340267, 6590 | 7.88532E−05 |
| Process | | peptidase activity | | | |
| Biological | GO:0051897 | Positive regulation of | 9 | 1116, 6363, 6366, 5795, 1440, 6348, 9518, 114897, 1956 | 0.000265725 |
| Process | | protein kinase b | | | |
| | | signaling | | | |
| Biological | GO:0002548 | Monocyte chemotaxis | 6 | 6362, 6363, 6366, 6367, 6348, 3570 | 0.001118472 |
| Process | | | | | |
| Biological | GO:0006935 | Chemotaxis | 8 | 6373, 6362, 58191, 5919, 6367, 6348, 3491, 3627 | 0.001687375 |
| Process | | | | | |
| Biological | GO:0006955 | Immune response | 14 | 6362, 6892, 6363, 7133, 6366, 7850, 6367, 1440, 6590, | 0.002716477 |
| Process | | | | 1118, 567, 6348, 10563, 9966 | |
| Biological | GO:0010818 | T cell chemotaxis | 4 | 6373, 58191, 6348, 3627 | 0.008871159 |
| Process | | | | | |
| Biological | GO:0045071 | Negative regulation of | 5 | 3428, 4599, 9636, 3609, 6590 | 0.017056547 |
| Process | | viral genome replication | | | |
| Biological | GO:0071222 | Cellular response to | 8 | 6373, 11093, 7133, 3929, 567, 1440, 10563, 3627 | 0.017056547 |
| Process | | lipopolysaccharide | | | |
| Biological | GO:0045087 | Innate immune response | 14 | 5266, 481, 353514, 6590, 3428, 54210, 10406, 1401, 5919, | 0.018326624 |
| Process | | | | 3929, 567, 6039, 4599, 717 | |
| Biological | GO:0071356 | Cellular response to | 7 | 6362, 1116, 11093, 6363, 6366, 6367, 6348 | 0.019335815 |
| Process | | tumor necrosis factor | | | |
| Biological | GO:0050829 | Defense response to | 6 | 5919, 3929, 567, 4069, 6039, 3570 | 0.023777112 |
| Process | | gram-negative | | | |
| | | bacterium | | | |
| Biological | GO:0071346 | Cellular response to | 6 | 6362, 11093, 6363, 6366, 6367, 6348 | 0.026213782 |
| Process | | interferon-gamma | | | |
| Biological | GO:0001934 | Positive regulation of | 8 | 177, 5919, 2147, 2277, 8313, 3491, 1956, 8074 | 0.030180298 |
| Process | | protein phosphorylation | | | |
| Biological | GO:0019221 | Cytokine-mediated | 7 | 7850, 5795, 1440, 3563, 3570, 1271, 353514 | 0.032027121 |
| Process | | signaling pathway | | | |
| Biological | GO:0019731 | Antibacterial humoral | 5 | 10406, 5266, 567, 6039, 6590 | 0.032234732 |
| Process | | response | | | |
| Biological | GO:0032760 | Positive regulation of | 6 | 177, 6363, 3929, 5795, 6348, 353514 | 0.033206449 |
| Process | | tumor necrosis factor | | | |
| | | production | | | |
| Biological | GO:0045860 | Positive regulation of | 5 | 6363, 6366, 8313, 2056, 3491 | 0.033785047 |
| Process | | protein kinase activity | | | |
| Biological | GO:0048245 | Eosinophil chemotaxis | 4 | 6362, 6366, 6367, 6348 | 0.034191277 |
| Process | | | | | |
| Biological | GO:0032731 | Positive regulation of | 5 | 3428, 177, 6363, 6348, 353514 | 0.035449894 |
| Process | | interleukin-1 beta | | | |
| | | production | | | |
| Biological | GO:0007267 | Cell-cell signaling | 8 | 6373, 6362, 6366, 6367, 6348, 9518, 10563, 3627 | 0.037272732 |
| Process | | | | | |

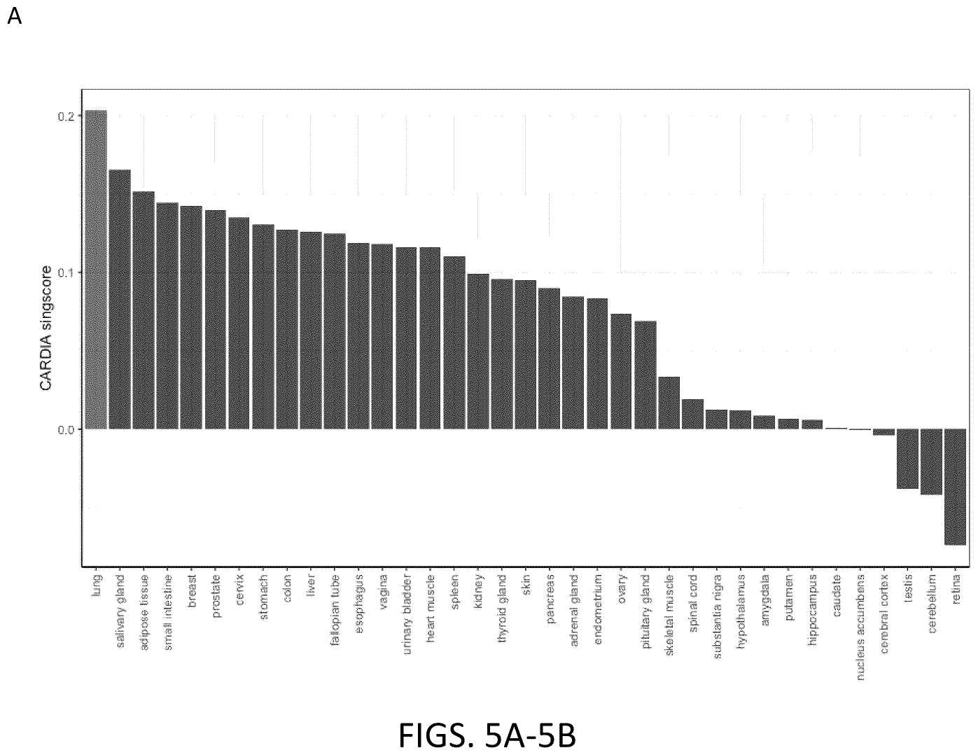

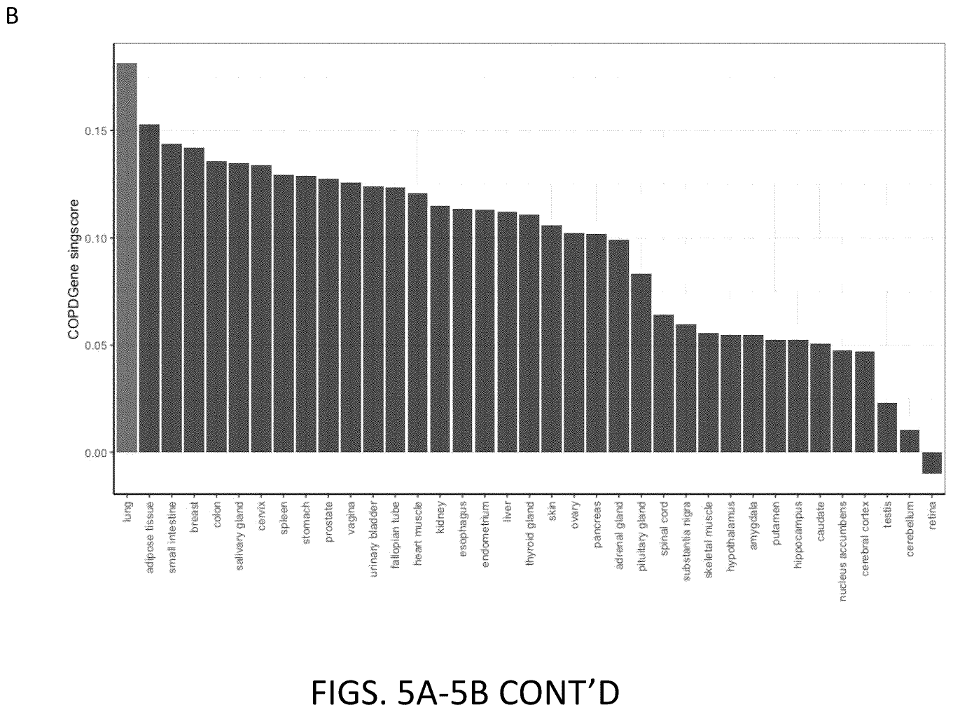

Deconvolution analysis of the proteins showed that the gene transcripts of the proteins positively associated with QIA were most commonly expressed in the lungs, further suggesting that plasma proteomic biomarkers may be beneficial in understanding disease processes in the lungs (FIG. 5).

For sensitivity analysis, given that smoking is an important risk factor for QIA and many advanced lung diseases, and that COPDGene is an older, ever-smoking cohort, we restricted the CARDIA population to the 876 ever-smokers (former and current smokers) who were at least 50 years old (minimum recruitment age of COPDGene, Table 9) and ran the analyses on the 842 proteins associated with QIA in COPDGene.

TABLE 9
Baseline characteristics of the older ever-
smoker and never-smoker subsets in CARDIA

| | Older ever- | Never- |
|---|---|---|
| | smokers | smokers |
| | N = 876 | N = 1490 |
| Age, mean ± SD | 52.8 ± 1.8 | 50.1 ± 3.6 |
| Females, N(%) | 498 (56.8) | 846 (56.8) |
| Males, N(%) | 378 (43.2) | 644 (43.2) |
| Non-Hispanic White, N(%) | 487 (55.6) | 832 (55.8) |
| Black, N(%) | 389 (44.4) | 658 (44.2) |
| Current smoker, N(%) | 475 (54.2) | — |
| Former smoker, N(%) | 401 (45.8) | — |
| Pack-years, mean ± SD | 12.1 ± 12.8 | — |
| BMI, mean ± SD | 29.9 ± 7.0 | 30.5 ± 7.2 |
| Percent QIA, mean ± SD | 2.8 ± 5.3 | 2.5 ± 4.7 |
| Percent quantitative emphysema, mean ± SD | 1.3 ± 2.9 | 0.9 ± 1.6 |
| Percent predicted FEV1, mean ± SD | 92.9 ± 15.3 | 93.8 ± 13.8 |
| Percent predicted FVC, mean ± SD | 96.3 ± 14.5 | 95.3 ± 13.5 |

After adjusting for multiple testing in the smaller sample, there were 201 proteins (195 GeneIDs) independently associated with QIA (Table 10).

TABLE 10
COPDGene sample set proteins independently associated with QIA

| | | | Percent | Upper | Lower | | |
|---|---|---|---|---|---|---|---|
| | Entrez gene | | QIA per | 95% | 95% | p- | FDR p- |
| Protein name | name | UniProt ID | ln(RFU) | CI | CI | value | value |
| Complement factor H | CFH | P08603 | 7.21 | 2.5 | 11.92 | 2.75E−03 | 1.82E−02 |
| Fibrinogen | FGA\|FGB\|FGG | P02671\|P02675\|P02679 | 6.07 | 1.41 | 10.73 | 1.07E−02 | 4.67E−02 |
| Complement factor B | CFB | P00751 | 4.59 | 1.96 | 7.22 | 6.49E−04 | 7.25E−03 |
| Antileukoproteinase | SLPI | P03973 | 4.56 | 2.64 | 6.49 | 3.83E−06 | 4.76E−04 |
| Neurofascin | NFASC | O94856 | 4.41 | 2.47 | 6.34 | 9.02E−06 | 6.41E−04 |
| Neuropilin-2 | NRP2 | O60462 | 4.2 | 1.86 | 6.54 | 4.46E−04 | 5.58E−03 |
| Fibrinogen gamma | FGG | P02679 | 3.96 | 1.51 | 6.41 | 1.59E−03 | 1.25E−02 |
| chain | | | | | | | |
| Insulin-like growth | IGFBP4 | P22692 | 3.8 | 2.12 | 5.48 | 9.79E−06 | 6.41E−04 |
| factor-binding protein | | | | | | | |
| 4 | | | | | | | |
| C-X-C motif | CXCL16 | Q9H2A7 | 3.56 | 1.77 | 5.35 | 1.02E−04 | 2.18E−03 |
| chemokine 16 | | | | | | | |
| Atrial natriuretic | NPPA | P01160 | 3.42 | 1.51 | 5.34 | 4.68E−04 | 5.70E−03 |
| factor | | | | | | | |
| Serine/arginine-rich | SRSF7 | Q16629 | 3.3 | 1.5 | 5.1 | 3.38E−04 | 4.64E−03 |
| splicing factor 7 | | | | | | | |
| Tumor necrosis factor | TNFSF15 | O95150 | 3.28 | 1.54 | 5.03 | 2.34E−04 | 3.86E−03 |
| ligand superfamily | | | | | | | |
| member 15 | | | | | | | |
| Serine/arginine-rich | SRSF6 | Q13247 | 3.25 | 1.82 | 4.68 | 8.66E−06 | 6.41E−04 |
| splicing factor 6 | | | | | | | |
| RNA-binding protein | EWSR1 | Q01844 | 3.17 | 1.41 | 4.93 | 4.32E−04 | 5.49E−03 |
| EWS | | | | | | | |
| Immunoglobulin G | IGHG1 | P01857 | 3.16 | 1 | 5.31 | 4.17E−03 | 2.45E−02 |
| Serpin B8 | SERPINB8 | P50452 | 3.13 | 1.75 | 4.51 | 9.36E−06 | 6.41E−04 |
| Leukocyte | LILRA5 | A6NI73 | 3.11 | 1.64 | 4.58 | 3.54E−05 | 1.19E−03 |
| immunoglobulin-like | | | | | | | |
| receptor subfamily A | | | | | | | |
| member 5 | | | | | | | |
| Cystatin-C | CST3 | P01034 | 3.08 | 1.49 | 4.67 | 1.49E−04 | 2.77E−03 |
| Nidogen-1 | NID1 | P14543 | 3.08 | 1.45 | 4.72 | 2.19E−04 | 3.74E−03 |
| Gamma-glutamyl | GGH | Q92820 | 3.01 | 1.57 | 4.44 | 4.25E−05 | 1.25E−03 |
| hydrolase | | | | | | | |
| Complement C1q | C1QTNF1 | Q9BXJ1 | 3 | 1.02 | 4.99 | 3.07E−03 | 1.94E−02 |
| tumor necrosis factor- | | | | | | | |
| related protein 1 | | | | | | | |
| Insulin-like growth | IGFBP7 | Q16270 | 3 | 0.77 | 5.24 | 8.50E−03 | 4.04E−02 |
| factor-binding protein 7 | | | | | | | |
| Plastin-2 | LCP1 | P13796 | 2.99 | 0.8 | 5.18 | 7.41E−03 | 3.65E−02 |
| Desmocollin-2 | DSC2 | Q02487 | 2.98 | 1.44 | 4.51 | 1.50E−04 | 2.77E−03 |
| Ganglioside GM2 | GM2A | P17900 | 2.97 | 1.58 | 4.36 | 3.15E−05 | 1.17E−03 |
| activator | | | | | | | |
| Fibulin-5 | FBLN5 | Q9UBX5 | 2.94 | 1.24 | 4.64 | 7.08E−04 | 7.54E−03 |
| Ephrin-A4 | EFNA4 | P52798 | 2.86 | 1.09 | 4.64 | 1.62E−03 | 1.25E−02 |
| Heat shock 70 kDa | HSPA1B | P0DMV9 | 2.84 | 1.31 | 4.38 | 2.99E−04 | 4.42E−03 |
| protein 1B | | | | | | | |
| Spondin-1 | SPON1 | Q9HCB6 | 2.83 | 0.9 | 4.76 | 4.06E−03 | 2.40E−02 |
| Acidic leucine-rich | ANP32B | Q92688 | 2.8 | 1.03 | 4.58 | 2.02E−03 | 1.47E−02 |
| nuclear | | | | | | | |
| phosphoprotein 32 | | | | | | | |
| family member B | | | | | | | |
| Peptidyl-prolyl cis- | PPIC | P45877 | 2.79 | 1.19 | 4.39 | 6.33E−04 | 7.19E−03 |
| trans isomerase C | | | | | | | |
| B melanoma antigen 2 | BAGE2 | Q86Y30 | 2.79 | 1.28 | 4.31 | 3.01E−04 | 4.42E−03 |
| Collagen alpha- | COL28A1 | Q2UY09 | 2.75 | 1.17 | 4.34 | 6.61E−04 | 7.25E−03 |
| 1(XXVIII) chain | | | | | | | |
| Oncoprotein-induced | OIT3 | Q8WWZ8 | 2.75 | 1.5 | 4 | 1.66E−05 | 8.79E−04 |
| transcript 3 protein | | | | | | | |
| Collagen alpha-3(VI) | COL6A3 | P12111 | 2.73 | 1.24 | 4.22 | 3.51E−04 | 4.64E−03 |
| chain: Bovine | | | | | | | |
| pancreatic trypsin | | | | | | | |
| inhibitor/Kunitz | | | | | | | |
| inhibitor domain, | | | | | | | |
| isoform 1 | | | | | | | |
| Transmembrane | TMED10 | P49755 | 2.73 | 1.53 | 3.93 | 9.42E−06 | 6.41E−04 |
| emp24 domain- | | | | | | | |
| containing protein 10 | | | | | | | |
| Delta-like protein 1 | DLL1 | O00548 | 2.7 | 1.04 | 4.36 | 1.49E−03 | 1.23E−02 |
| Endoplasmic | ERLEC1 | Q96DZ1 | 2.66 | 1.14 | 4.17 | 6.05E−04 | 6.97E−03 |
| reticulum lectin 1 | | | | | | | |
| C-C motif chemokine | CCL21 | O00585 | 2.59 | 1.44 | 3.75 | 1.21E−05 | 7.39E−04 |
| 21 | | | | | | | |
| Tumor necrosis factor | TNFRSF17 | Q02223 | 2.54 | 1.16 | 3.92 | 3.22E−04 | 4.50E−03 |
| receptor superfamily | | | | | | | |
| member 17 | | | | | | | |
| Thioredoxin domain- | TXNDC5 | Q8NBS9 | 2.54 | 1.12 | 3.96 | 4.81E−04 | 5.77E−03 |
| containing protein 5 | | | | | | | |
| Beta-2-microglobulin | B2M | P61769 | 2.52 | 1.37 | 3.67 | 2.03E−05 | 8.79E−04 |
| Heat shock cognate | HSPA8 | P11142 | 2.48 | 0.94 | 4.02 | 1.62E−03 | 1.25E−02 |
| 71 kDa protein | | | | | | | |
| WAP four-disulfide | WFDC2 | Q14508 | 2.46 | 1.27 | 3.64 | 5.07E−05 | 1.44E−03 |
| core domain protein 2 | | | | | | | |
| Heat shock 70 kDa | HSPA1A | PODMV8 | 2.43 | 0.84 | 4.01 | 2.77E−03 | 1.82E−02 |
| protein 1A | | | | | | | |
| Tolloid-like protein 1 | TLL1 | O43897 | 2.43 | 0.76 | 4.1 | 4.38E−03 | 2.50E−02 |
| Triggering receptor | TREM1 | Q9NP99 | 2.41 | 1.21 | 3.61 | 8.36E−05 | 1.87E−03 |
| expressed on myeloid | | | | | | | |
| cells 1 | | | | | | | |
| Calsyntenin-3 | CLSTN3 | Q9BQT9 | 2.41 | 1.21 | 3.62 | 8.98E−05 | 1.96E−03 |
| Lipopolysaccharide- | LBP | P18428 | 2.4 | 1.39 | 3.41 | 3.69E−06 | 4.76E−04 |
| binding protein | | | | | | | |
| Lamina-associated | TMPO | P42167 | 2.35 | 1.28 | 3.43 | 1.79E−05 | 8.79E−04 |
| polypeptide 2, | | | | | | | |
| isoforms beta/gamma | | | | | | | |
| Erythropoietin | EPO | P01588 | 2.34 | 1.48 | 3.19 | 1.04E−07 | 8.83E−05 |
| Ephrin type-B | EPHB2 | P29323 | 2.26 | 0.57 | 3.94 | 8.63E−03 | 4.09E−02 |
| receptor 2 | | | | | | | |
| C-X-C motif | CXCL13 | O43927 | 2.25 | 1.03 | 3.46 | 3.16E−04 | 4.49E−03 |
| chemokine 13 | | | | | | | |
| Tryptophan--tRNA | WARS1 | P23381 | 2.24 | 0.55 | 3.92 | 9.24E−03 | 4.33E−02 |
| ligase, cytoplasmic | | | | | | | |
| Folate receptor beta | FOLR2 | P14207 | 2.22 | 0.53 | 3.9 | 1.03E−02 | 4.66E−02 |
| Holo- | TCN2 | P20062 | 2.22 | 0.74 | 3.71 | 3.44E−03 | 2.11E−02 |
| Transcobalamin-2 | | | | | | | |
| NTF2-related export | NXT1 | Q9UKK6 | 2.21 | 0.9 | 3.53 | 9.95E−04 | 9.52E−03 |
| protein 1 | | | | | | | |
| Protein ABHD14A | ABHD14A | Q9BUJ0 | 2.2 | 0.84 | 3.56 | 1.59E−03 | 1.25E−02 |
| C-C motif chemokine | CCL18 | P55774 | 2.19 | 1.01 | 3.37 | 2.75E−04 | 4.26E−03 |
| 18 | | | | | | | |
| Semaphorin-6B | SEMA6B | Q9H3T3 | 2.19 | 0.53 | 3.84 | 9.65E−03 | 4.47E−02 |
| Retinoic acid receptor | RARRES2 | Q99969 | 2.18 | 0.63 | 3.74 | 6.06E−03 | 3.15E−02 |
| responder protein 2 | | | | | | | |
| Plexin-D1 | PLXND1 | Q9Y4D7 | 2.18 | 0.49 | 3.87 | 1.14E−02 | 4.84E−02 |
| Troponin T, cardiac | TNNT2 | P45379 | 2.18 | 1.14 | 3.21 | 3.84E−05 | 1.19E−03 |
| muscle | | | | | | | |
| Macrophage-capping | CAPG | P40121 | 2.15 | 1.13 | 3.17 | 3.90E−05 | 1.19E−03 |
| protein | | | | | | | |
| Syntaxin-12 | STX12 | Q86Y82 | 2.14 | 1.04 | 3.23 | 1.46E−04 | 2.77E−03 |
| Interleukin-9 | IL9 | P15248 | 2.13 | 0.74 | 3.52 | 2.72E−03 | 1.81E−02 |
| CUB domain- | CDCP1 | Q9H5V8 | 2.11 | 1.3 | 2.92 | 3.96E−07 | 1.14E−04 |
| containing protein 1 | | | | | | | |
| Vesicular integral- | LMAN2 | Q12907 | 2.11 | 0.49 | 3.73 | 1.07E−02 | 4.67E−02 |
| membrane protein | | | | | | | |
| VIP36 | | | | | | | |
| Fibroblast growth | FGF23 | Q9GZV9 | 2.1 | 1.29 | 2.91 | 4.00E−07 | 1.14E−04 |
| factor 23 | | | | | | | |
| Heat shock 70 kDa | HSPA1A | P0DMV8 | 2.07 | 0.59 | 3.56 | 6.27E−03 | 3.22E−02 |
| protein 1A | | | | | | | |
| BMP and activin | BAMBI | Q13145 | 2.05 | 0.59 | 3.52 | 6.10E−03 | 3.15E−02 |
| membrane-bound | | | | | | | |
| inhibitor | | | | | | | |
| homolog: Extracellular | | | | | | | |
| domain | | | | | | | |
| Marginal zone B- and | MZB1 | Q8WU39 | 2.04 | 1.08 | 3.01 | 3.58E−05 | 1.19E−03 |
| B1-cell-specific | | | | | | | |
| protein | | | | | | | |
| Leukocyte | LILRA5 | A6NI73 | 2.04 | 0.63 | 3.45 | 4.52E−03 | 2.55E−02 |
| immunoglobulin-like | | | | | | | |
| receptor subfamily A | | | | | | | |
| member 5 | | | | | | | |
| Interferon-induced | MX1 | P20591 | 2.03 | 1.22 | 2.83 | 9.52E−07 | 2.03E−04 |
| GTP-binding protein | | | | | | | |
| Mx1 | | | | | | | |
| Sushi, von Willebrand | SVEP1 | Q4LDE5 | 1.99 | 0.84 | 3.14 | 7.41E−04 | 7.79E−03 |
| factor type A, EGF | | | | | | | |
| and pentraxin domain- | | | | | | | |
| containing protein | | | | | | | |
| 1: EGF-like domains | | | | | | | |
| 4-6 | | | | | | | |
| Heat shock 70 kDa | HSPA1A | P0DMV8 | 1.99 | 0.58 | 3.39 | 5.58E−03 | 2.97E−02 |
| protein 1A | | | | | | | |
| Intercellular adhesion | ICAM1 | P05362 | 1.96 | 1 | 2.92 | 6.31E−05 | 1.62E−03 |
| molecule 1 | | | | | | | |
| Ephrin type-A | EPHA2 | P29317 | 1.94 | 0.55 | 3.33 | 6.33E−03 | 3.23E−02 |
| receptor 2 | | | | | | | |
| Transferrin receptor | TFRC | P02786 | 1.93 | 0.77 | 3.08 | 1.12E−03 | 1.01E−02 |
| protein 1: Extracellular | | | | | | | |
| domain | | | | | | | |
| Trefoil factor 3 | TFF3 | Q07654 | 1.93 | 0.78 | 3.09 | 1.02E−03 | 9.67E−03 |
| Kallikrein-10 | KLK10 | O43240 | 1.93 | 0.74 | 3.12 | 1.46E−03 | 1.23E−02 |
| Alpha-1- | SERPINA3 | P01011 | 1.93 | 0.65 | 3.21 | 3.21E−03 | 2.01E−02 |
| antichymotrypsin | | | | | | | |
| complex | | | | | | | |
| Neuroblastoma | NBL1 | P41271 | 1.92 | 0.76 | 3.08 | 1.21E−03 | 1.06E−02 |
| suppressor of | | | | | | | |
| tumorigenicity 1 | | | | | | | |
| Vascular endothelial | VEGFD | O43915 | 1.92 | 0.98 | 2.87 | 6.97E−05 | 1.65E−03 |
| growth factor D | | | | | | | |
| Small ubiquitin- | SUMO2 | P61956 | 1.91 | 0.74 | 3.09 | 1.44E−03 | 1.23E−02 |
| related modifier 2 | | | | | | | |
| T-cell | TIMD4 | Q96H15 | 1.9 | 0.77 | 3.03 | 9.78E−04 | 9.47E−03 |
| immunoglobulin and | | | | | | | |
| mucin domain- | | | | | | | |
| containing protein 4 | | | | | | | |
| Ribonuclease K6 | RNASE6 | Q93091 | 1.89 | 0.76 | 3.02 | 1.06E−03 | 9.80E−03 |
| IGF-like family | IGFLR1 | Q9H665 | 1.88 | 0.93 | 2.84 | 1.20E−04 | 2.44E−03 |
| receptor 1 | | | | | | | |
| Sushi, von Willebrand | SVEP1 | Q4LDE5 | 1.87 | 0.57 | 3.16 | 4.72E−03 | 2.61E−02 |
| factor type A, EGF | | | | | | | |
| and pentraxin domain- | | | | | | | |
| containing protein | | | | | | | |
| 1: Sushi 15-18 | | | | | | | |
| Elafin | PI3 | P19957 | 1.86 | 0.96 | 2.76 | 5.76E−05 | 1.53E−03 |
| ADAMTS-like | ADAMTSL2 | Q86TH1 | 1.81 | 0.49 | 3.12 | 6.99E−03 | 3.46E−02 |
| protein 2 | | | | | | | |
| Transformer-2 protein | TRA2B | P62995 | 1.81 | 0.64 | 2.99 | 2.48E−03 | 1.73E−02 |
| homolog beta | | | | | | | |
| Heat shock 70 kDa | HSPA1A | P0DMV8 | 1.79 | 0.57 | 3.01 | 3.99E−03 | 2.38E−02 |
| protein 1A | | | | | | | |
| Protein CYR61 | CCN1 | O00622 | 1.76 | 0.55 | 2.97 | 4.54E−03 | 2.55E−02 |
| Lysozyme C | LYZ | P61626 | 1.75 | 0.66 | 2.83 | 1.63E−03 | 1.25E−02 |
| Guanylate-binding | GBP1 | P32455 | 1.73 | 0.66 | 2.8 | 1.54E−03 | 1.24E−02 |
| protein 1 | | | | | | | |
| Macrophage | MMP12 | P39900 | 1.72 | 0.8 | 2.64 | 2.50E−04 | 4.02E−03 |
| metalloelastase | | | | | | | |
| Angiopoietin-2 | ANGPT2 | O15123 | 1.71 | 0.6 | 2.83 | 2.64E−03 | 1.77E−02 |
| Axin-2 | AXIN2 | Q9Y2T1 | 1.7 | 0.87 | 2.53 | 6.57E−05 | 1.62E−03 |
| WAP four-disulfide | WFDC1 | Q9HC57 | 1.7 | 0.4 | 2.99 | 1.04E−02 | 4.66E−02 |
| core domain protein 1 | | | | | | | |
| Complement C3d | C3 | P01024 | 1.69 | 0.5 | 2.88 | 5.46E−03 | 2.93E−02 |
| fragment | | | | | | | |
| DnaJ homolog | DNAJB12 | Q9NXW2 | | | | | 1.29E−02 |
| subfamily B member | | | | | | | |
| 12 | | | 1.69 | 0.64 | 2.75 | 1.69E−03 | |
| Prostaglandin-H2 D- | PTGDS | P41222 | 1.67 | 0.44 | 2.91 | 7.73E−03 | 3.79E−02 |
| isomerase | | | | | | | |
| Interleukin-1 receptor | IL1RN | P18510 | 1.67 | 0.43 | 2.9 | 8.23E−03 | 3.99E−02 |
| antagonist protein | | | | | | | |
| Programmed cell | PDCD5 | O14737 | 1.67 | 0.44 | 2.9 | 7.84E−03 | 3.82E−02 |
| death protein 5 | | | | | | | |
| Regenerating islet- | REG3A | Q06141 | 1.67 | 0.86 | 2.49 | 5.67E−05 | 1.53E−03 |
| derived protein 3- | | | | | | | |
| alpha | | | | | | | |
| Lactoperoxidase | LPO | P22079 | 1.67 | 0.88 | 2.46 | 3.65E−05 | 1.19E−03 |
| SCP2 sterol-binding | SCP2D1 | Q9UJQ7 | 1.63 | 0.81 | 2.45 | 1.07E−04 | 2.22E−03 |
| domain-containing | | | | | | | |
| protein 1 | | | | | | | |
| Trefoil factor 3 | TFF3 | Q07654 | 1.62 | 0.62 | 2.62 | 1.50E−03 | 1.23E−02 |
| C-C motif chemokine | CCL19 | Q99731 | 1.59 | 0.68 | 2.49 | 5.89E−04 | 6.88E−03 |
| 19 | | | | | | | |
| Granulocyte colony- | CSF3 | P09919 | 1.56 | 0.75 | 2.37 | 1.58E−04 | 2.87E−03 |
| stimulating factor | | | | | | | |
| Phospholipase A2, | PLA2G2A | P14555 | 1.54 | 0.6 | 2.49 | 1.35E−03 | 1.18E−02 |
| membrane associated | | | | | | | |
| Trefoil factor 2 | TFF2 | Q03403 | 1.54 | 0.62 | 2.46 | 1.04E−03 | 9.71E−03 |
| Gamma-interferon- | IFI16 | Q16666 | 1.54 | 0.5 | 2.59 | 3.65E−03 | 2.22E−02 |
| inducible protein | | | | | | | |
| 16: Isoform 2, | | | | | | | |
| Hematopoietic | | | | | | | |
| expression, interferon- | | | | | | | |
| inducible nature, and | | | | | | | |
| nuclear localization 1 | | | | | | | |
| Acyl-CoA-binding | DBI | P07108 | 1.52 | 0.38 | 2.66 | 9.13E−03 | 4.30E−02 |
| protein | | | | | | | |
| Thioredoxin reductase | TXNRD1 | Q16881 | 1.51 | 0.46 | 2.56 | 4.83E−03 | 2.64E−02 |
| 1, cytoplasmic | | | | | | | |
| Interleukin-3 receptor | IL3RA | P26951 | 1.49 | 0.36 | 2.63 | 1.02E−02 | 4.66E−02 |
| subunit alpha | | | | | | | |
| Thrombospondin-2 | THBS2 | P35442 | 1.48 | 0.63 | 2.33 | 7.03E−04 | 7.54E−03 |
| Tumor necrosis factor | TNFRSF1B | P20333 | 1.48 | 0.47 | 2.49 | 4.21E−03 | 2.46E−02 |
| receptor superfamily | | | | | | | |
| member 1B | | | | | | | |
| Golgi membrane | GOLM1 | Q8NBJ4 | 1.48 | 0.51 | 2.46 | 2.96E−03 | 1.91E−02 |
| protein 1 | | | | | | | |
| Growth/differentiation | GDF15 | Q99988 | 1.46 | 0.54 | 2.37 | 1.81E−03 | 1.35E−02 |
| factor 15 | | | | | | | |
| Kallikrein-13 | KLK13 | Q9UKR3 | 1.46 | 0.41 | 2.52 | 6.61E−03 | 3.33E−02 |
| Induced myeloid | MCL1 | Q07820 | 1.45 | 0.47 | 2.42 | 3.73E−03 | 2.26E−02 |
| leukemia cell | | | | | | | |
| differentiation protein | | | | | | | |
| Mcl-1 | | | | | | | |
| E-selectin | SELE | P16581 | 1.44 | 0.51 | 2.38 | 2.52E−03 | 1.73E−02 |
| Macrophage | MSR1 | P21757 | 1.44 | 0.49 | 2.38 | 2.94E−03 | 1.91E−02 |
| scavenger receptor | | | | | | | |
| types I and | | | | | | | |
| II: Extracellular | | | | | | | |
| domain | | | | | | | |
| Ribonuclease | RNASE1 | P07998 | 1.44 | 0.67 | 2.21 | 2.71E−04 | 4.26E−03 |
| pancreatic | | | | | | | |
| Gastrokine-2 | GKN2 | Q86XP6 | 1.33 | 0.61 | 2.04 | 2.89E−04 | 4.40E−03 |
| Receptor-transporting | RTP4 | Q96DX8 | 1.33 | 0.42 | 2.24 | 4.31E−03 | 2.50E−02 |
| protein 4 | | | | | | | |
| Ubiquitin-like protein | ISG15 | P05161 | 1.32 | 0.62 | 2.02 | 2.19E−04 | 3.74E−03 |
| ISG15 | | | | | | | |
| C-C motif chemokine | CCL15 | Q16663 | 1.27 | 0.3 | 2.24 | 1.03E−02 | 4.66E−02 |
| 15 | | | | | | | |
| Copper transport | ATOX1 | O00244 | 1.27 | 0.29 | 2.25 | 1.11E−02 | 4.79E−02 |
| protein ATOX1 | | | | | | | |
| C-X-C motif | CXCL10 | P02778 | 1.27 | 0.45 | 2.1 | 2.49E−03 | 1.73E−02 |
| chemokine 10 | | | | | | | |
| 60S ribosomal protein | RPL30 | P62888 | 1.27 | 0.37 | 2.18 | 5.68E−03 | 3.01E−02 |
| L30 | | | | | | | |
| Troponin I, fast | TNNI2 | P48788 | 1.26 | 0.56 | 1.96 | 4.56E−04 | 5.63E−03 |
| skeletal muscle | | | | | | | |
| Cadherin- | CDH11 | P55287 | 1.2 | 0.67 | 1.72 | 8.79E−06 | 6.41E−04 |
| 11: Extracellular | | | | | | | |
| domain | | | | | | | |
| Syntaxin-8 | STX8 | Q9UNK0 | 1.2 | 0.42 | 1.99 | 2.60E−03 | 1.76E−02 |
| Fibrinogen-like | FGL1 | Q08830 | 1.18 | 0.34 | 2.02 | 5.72E−03 | 3.01E−02 |
| protein 1 | | | | | | | |
| Serum amyloid A-2 | SAA2 | P0DJI9 | 1.11 | 0.42 | 1.79 | 1.54E−03 | 1.24E−02 |
| protein | | | | | | | |
| Chitotriosidase-1 | CHIT1 | Q13231 | 1.09 | 0.44 | 1.74 | 1.13E−03 | 1.01E−02 |
| Platelet-derived | PDGFRL | Q15198 | 1.07 | 0.36 | 1.79 | 3.43E−03 | 2.11E−02 |
| growth factor | | | | | | | |
| receptor-like protein | | | | | | | |
| BPI fold-containing | BPIFB1 | Q8TDL5 | 1.02 | 0.29 | 1.75 | 6.03E−03 | 3.15E−02 |
| family B member 1 | | | | | | | |
| N-terminal pro-BNP | NPPB | P16860 | 0.86 | 0.32 | 1.4 | 1.81E−03 | 1.35E−02 |
| Beta-defensin 4A | DEFB4A | O15263 | 0.81 | 0.41 | 1.21 | 7.52E−05 | 1.73E−03 |
| C-reactive protein | CRP | P02741 | 0.65 | 0.2 | 1.1 | 4.42E−03 | 2.51E−02 |
| Serum amyloid A-1 | SAA1 | P0DJI8 | 0.65 | 0.17 | 1.13 | 8.48E−03 | 4.04E−02 |
| protein | | | | | | | |
| Zymogen granule | ZG16 | O60844 | 0.28 | 0.14 | 0.42 | 6.64E−05 | 1.62E−03 |
| membrane protein 16 | | | | | | | |
| Signal peptide, CUB | SCUBE1 | Q8IWY4 | −0.81 | −1.37 | −0.25 | 4.91E−03 | 2.67E−02 |
| and EGF-like domain- | | | | | | | |
| containing protein 1 | | | | | | | |
| Glypican-3 | GPC3 | P51654 | −1.24 | −2.18 | −0.29 | 1.05E−02 | 4.66E−02 |
| Chondrocalcin | COL2A1 | P02458 | −1.3 | −1.98 | −0.63 | 1.70E−04 | 3.01E−03 |
| Protein MENT | MENT | Q9BUN1 | −1.36 | −2.37 | −0.35 | 8.34E−03 | 4.01E−02 |
| Histone-lysine N- | EHMT2 | Q96KQ7 | −1.4 | −2.48 | −0.32 | 1.13E−02 | 4.83E−02 |
| methyltransferase | | | | | | | |
| EHMT2 | | | | | | | |
| Sodium/potassium- | ATP1B1 | P05026 | −1.46 | −2.42 | −0.49 | 3.08E−03 | 1.94E−02 |
| transporting ATPase | | | | | | | |
| subunit beta-1 | | | | | | | |
| Neurocan core protein | NCAN | O14594 | −1.64 | −2.9 | −0.39 | 1.03E−02 | 4.66E−02 |
| Cartilage intermediate | CILP2 | Q8IUL8 | −1.79 | −3.08 | −0.5 | 6.77E−03 | 3.37E−02 |
| layer protein 2 | | | | | | | |
| Group XIIB secretory | PLA2G12B | Q9BX93 | −1.88 | −3.19 | −0.57 | 4.84E−03 | 2.64E−02 |
| phospholipase A2-like | | | | | | | |
| protein | | | | | | | |
| Ficolin-3 | FCN3 | O75636 | −2.06 | −3.43 | −0.69 | 3.30E−03 | 2.06E−02 |
| Fibroblast growth | FGFR3 | P22607 | −2.07 | −3.41 | −0.72 | 2.60E−03 | 1.76E−02 |
| factor receptor | | | | | | | |
| 3: Extracellular | | | | | | | |
| domain | | | | | | | |
| Muellerian-inhibiting | AMH | P03971 | −2.08 | −3.67 | −0.48 | 1.08E−02 | 4.67E−02 |
| factor | | | | | | | |
| Solute carrier family | SLC22A16 | Q86VW1 | −2.14 | −3.68 | −0.6 | 6.46E−03 | 3.28E−02 |
| 22 member 16 | | | | | | | |
| Interleukin-6 receptor | IL6R | P08887 | −2.14 | −3.53 | −0.76 | 2.51E−03 | 1.73E−02 |
| subunit alpha | | | | | | | |
| Proprotein convertase | PCSK7 | Q16549 | −2.2 | −3.51 | −0.88 | 1.11E−03 | 1.01E−02 |
| subtilisin/kexin type 7 | | | | | | | |
| Anthrax toxin | ANTXR1 | Q9H6X2 | −2.24 | −3.65 | −0.83 | 1.92E−03 | 1.41E−02 |
| receptor 1 | | | | | | | |
| Inositol | BPNT2 | Q9NX62 | −2.37 | −3.91 | −0.84 | 2.48E−03 | 1.73E−02 |
| monophosphatase 3 | | | | | | | |
| Thrombin | F2 | P00734 | −2.41 | −3.72 | −1.09 | 3.54E−04 | 4.64E−03 |
| Neural cell adhesion | NCAM1 | P13591 | −2.5 | −4.42 | −0.59 | 1.06E−02 | 4.67E−02 |
| molecule 1, 120 kDa | | | | | | | |
| isoform | | | | | | | |
| Apolipoprotein M | APOM | O95445 | −2.52 | −4.24 | −0.81 | 3.95E−03 | 2.37E−02 |
| Receptor-type | PTPRD | P23468 | −2.55 | −4.16 | −0.94 | 1.92E−03 | 1.41E−02 |
| tyrosine-protein | | | | | | | |
| phosphatase delta | | | | | | | |
| Cell adhesion | CDON | Q4KMG0 | −2.57 | −4.43 | −0.71 | 6.76E−03 | 3.37E−02 |
| molecule- | | | | | | | |
| related/down- | | | | | | | |
| regulated by | | | | | | | |
| oncogenes | | | | | | | |
| Ciliary neurotrophic | CNTFR | P26992 | −2.57 | −4.33 | −0.81 | 4.35E−03 | 2.50E−02 |
| factor receptor subunit | | | | | | | |
| alpha | | | | | | | |
| E3 ubiquitin-protein | RNF146 | Q9NTX7 | −2.7 | −4.81 | −0.6 | 1.18E−02 | 4.98E−02 |
| ligase RNF146 | | | | | | | |
| N-acetylmuramoyl-L- | PGLYRP2 | Q96PD5 | −2.75 | −4.82 | −0.67 | 9.57E−03 | 4.46E−02 |
| alanine amidase | | | | | | | |
| Matrix-remodeling- | MXRA8 | Q9BRK3 | −2.78 | −4.42 | −1.13 | 9.54E−04 | 9.45E−03 |
| associated protein | | | | | | | |
| 8: Extracellular | | | | | | | |
| domain | | | | | | | |
| Growth/differentiation | GDF2 | Q9UK05 | −2.79 | −4.56 | −1.02 | 2.08E−03 | 1.50E−02 |
| factor 2 | | | | | | | |
| Platelet endothelial | PEAR1 | Q5VY43 | −2.8 | −4.93 | −0.66 | 1.04E−02 | 4.66E−02 |
| aggregation receptor | | | | | | | |
| 1: Extracellular | | | | | | | |
| domain | | | | | | | |
| Myosin regulatory | MYL7 | Q01449 | −2.82 | −4.46 | −1.18 | 7.70E−04 | 7.83E−03 |
| light chain 2, atrial | | | | | | | |
| isoform | | | | | | | |
| Hedgehog-interacting | HHIP | Q96QV1 | −2.82 | −4.69 | −0.96 | 2.98E−03 | 1.91E−02 |
| protein | | | | | | | |
| Carbonic anhydrase 4 | CA4 | P22748 | −2.9 | −5.13 | −0.67 | 1.08E−02 | 4.67E−02 |
| Prostaglandin F2 | PTGFRN | Q9P2B2 | −2.95 | −4.3 | −1.6 | 1.96E−05 | 8.79E−04 |
| receptor negative | | | | | | | |
| regulator | | | | | | | |
| E3 ubiquitin-protein | DTX1 | Q86Y01 | −2.95 | −4.85 | −1.05 | 2.36E−03 | 1.69E−02 |
| ligase DTX1 | | | | | | | |
| Interleukin-1 receptor | IL1R2 | P27930 | −2.96 | −4.59 | −1.32 | 4.06E−04 | 5.24E−03 |
| type 2 | | | | | | | |
| Receptor-type | PTPRJ | Q12913 | −3.07 | −4.86 | −1.29 | 7.51E−04 | 7.80E−03 |
| tyrosine-protein | | | | | | | |
| phosphatase eta | | | | | | | |
| NT-3 growth factor | NTRK3 | Q16288 | −3.12 | −4.94 | −1.31 | 7.71E−04 | 7.83E−03 |
| receptor | | | | | | | |
| Leucine-rich repeats | LRIG3 | Q6UXM1 | −3.34 | −5.26 | −1.42 | 6.64E−04 | 7.25E−03 |
| and immunoglobulin- | | | | | | | |
| like domains protein 3 | | | | | | | |
| Beta-1,4- | B4GALT6 | Q9UBX8 | −3.35 | −4.89 | −1.82 | 2.06E−05 | 8.79E−04 |
| galactosyltransferase 6 | | | | | | | |
| Semaphorin-3G | SEMA3G | Q9NS98 | −3.37 | −5.11 | −1.64 | 1.40E−04 | 2.77E−03 |
| Plasma kallikrein | KLKB1 | P03952 | −3.37 | −5.4 | −1.34 | 1.18E−03 | 1.05E−02 |
| Collagen alpha- | COL13A1 | Q5TAT6 | −3.46 | −5.35 | −1.57 | 3.49E−04 | 4.64E−03 |
| 1(XIII) chain | | | | | | | |
| Alpha-2-HS- | AHSG | P02765 | −3.49 | −6.17 | −0.81 | 1.06E−02 | 4.67E−02 |
| glycoprotein | | | | | | | |
| Bifunctional heparan | NDST1 | P52848 | −3.62 | −6.14 | −1.09 | 5.05E−03 | 2.72E−02 |
| sulfate N- | | | | | | | |
| deacetylase/N- | | | | | | | |
| sulfotransferase 1 | | | | | | | |
| Contactin-1 | CNTN1 | Q12860 | −3.64 | −5.71 | −1.57 | 5.78E−04 | 6.84E−03 |
| Cholinesterase | BCHE | P06276 | −3.73 | −5.45 | −2.01 | 2.27E−05 | 9.20E−04 |
| Heparan-sulfate 6-O- | HS6ST3 | Q8IZP7 | −3.79 | −6.12 | −1.46 | 1.47E−03 | 1.23E−02 |
| sulfotransferase 3 | | | | | | | |
| Coxsackievirus and | CXADR | P78310 | −3.86 | −6.24 | −1.49 | 1.47E−03 | 1.23E−02 |
| adenovirus receptor | | | | | | | |
| B melanoma antigen 3 | BAGE3 | Q86Y29 | −3.89 | −6.18 | −1.6 | 9.11E−04 | 9.13E−03 |
| BDNF/NT-3 growth | NTRK2 | Q16620 | −4.17 | −6.06 | −2.28 | 1.63E−05 | 8.79E−04 |
| factors receptor | | | | | | | |
| Contactin-4 | CNTN4 | Q8IWV2 | −4.21 | −6.18 | −2.24 | 3.02E−05 | 1.17E−03 |
| N-acetylglucosamine- | NAGPA | Q9UK23 | −4.3 | −6.11 | −2.48 | 3.91E−06 | 4.76E−04 |
| 1-phosphodiester | | | | | | | |
| alpha-N- | | | | | | | |
| acetylglucosaminidase | | | | | | | |
| Neuropeptide S | NPS | P0C0P6 | −4.48 | −7.13 | 1.82 | 9.74E−04 | 9.47E−03 |
| Alpha-2-antiplasmin | SERPINF2 | P08697 | −4.79 | −8.11 | −1.48 | 4.67E−03 | 2.60E−02 |
| Mannosyl- | MAN1A2 | O60476 | −4.83 | −7.45 | −2.22 | 3.06E−04 | 4.43E−03 |
| oligosaccharide 1,2- | | | | | | | |
| alpha-mannosidase IB | | | | | | | |
| Epidermal growth | EGFR | P00533 | −5.17 | −7.92 | −2.42 | 2.36E−04 | 3.86E−03 |
| factor receptor | | | | | | | |

Next, as QIA is present even in participants without a history of cigarette use, we ran the analyses restricted to the subset of 1,490 never-smokers in CARDIA and found that 49 of those proteins (48 GeneIDs) were independently associated with QIA (Table 11).

TABLE 11
Proteins independently associated with QIA in CARDIA never-smokers

| | | | Percent | | | | FDR |
|---|---|---|---|---|---|---|---|
| | Entrez | | QIA per | Upper | Lower | p- | p- |
| Protein name | gene name | UniProt ID | ln(RFU) | 95% CI | 95% CI | value | value |
| HLA class I | HLA-G | P17693 | 4.02 | 2 | 6.04 | 9.96E−05 | 1.21E−02 |
| histocompatibility | | | | | | | |
| antigen, alpha | | | | | | | |
| chain G | | | | | | | |
| Receptor- | RIPK2 | O43353 | 2.6 | 1.52 | 3.68 | 2.40E−06 | 2.05E−03 |
| interacting | | | | | | | |
| serine/threonine- | | | | | | | |
| protein kinase 2 | | | | | | | |
| Lumican | LUM | P51884 | 2.38 | 1.05 | 3.71 | 4.66E−04 | 2.06E−02 |
| Matrix Gla protein | MGP | P08493 | 2.37 | 1.02 | 3.71 | 5.59E−04 | 2.27E−02 |
| Heat shock 70 | HSPA1A | P0DMV8 | 2.3 | 0.84 | 3.77 | 2.05E−03 | 4.05E−02 |
| kDa protein 1A | | | | | | | |
| Switch-associated | SWAP70 | Q9UH65 | 2.28 | 0.96 | 3.6 | 7.33E−04 | 2.60E−02 |
| protein 70 | | | | | | | |
| Heat shock 70 | HSPA1A | P0DMV8 | 2.12 | 0.9 | 3.35 | 6.84E−04 | 2.53E−02 |
| kDa protein 1A | | | | | | | |
| Tumor necrosis | TNFSF15 | O95150 | 2.02 | 0.7 | 3.34 | 2.65E−03 | 4.60E−02 |
| factor ligand | | | | | | | |
| superfamily | | | | | | | |
| member 15 | | | | | | | |
| Insulin-like | IGFBP4 | P22692 | 1.96 | 0.73 | 3.19 | 1.84E−03 | 3.92E−02 |
| growth factor- | | | | | | | |
| binding protein 4 | | | | | | | |
| Ran-binding | RANBP3 | Q9H6Z4 | 1.83 | 0.74 | 2.93 | 1.01E−03 | 3.20E−02 |
| protein 3 | | | | | | | |
| Laminin subunit | LAMC2 | Q13753 | 1.82 | 1 | 2.65 | 1.57E−05 | 6.67E−03 |
| gamma-2 | | | | | | | |
| C-X-C motif | CXCL13 | O43927 | 1.69 | 0.89 | 2.5 | 3.98E−05 | 7.70E−03 |
| chemokine 13 | | | | | | | |
| Thrombospondin-4 | THBS4 | P35443 | 1.64 | 0.59 | 2.68 | 2.13E−03 | 4.12E−02 |
| Tripeptidyl- | TPP1 | O14773 | 1.62 | 0.66 | 2.57 | 9.36E−04 | 3.07E−02 |
| peptidase 1 | | | | | | | |
| Macrophage- | CAPG | P40121 | 1.55 | 0.81 | 2.3 | 4.52E−05 | 7.70E−03 |
| capping protein | | | | | | | |
| Thrombospondin-3 | THBS3 | P49746 | 1.51 | 0.65 | 2.38 | 6.13E−04 | 2.37E−02 |
| Myosin light chain | MYL6B | P14649 | 1.47 | 0.64 | 2.29 | 4.84E−04 | 2.06E−02 |
| 6B | | | | | | | |
| Torsin-1A- | TOR1AIP1 | Q5JTV8 | 1.46 | 0.58 | 2.35 | 1.18E−03 | 3.35E−02 |
| interacting | | | | | | | |
| protein | | | | | | | |
| 1: Perinuclear | | | | | | | |
| domain | | | | | | | |
| Calsyntenin-3 | CLSTN3 | Q9BQT9 | 1.41 | 0.66 | 2.17 | 2.40E−04 | 1.59E−02 |
| Lamina- | TMPO | P42167 | 1.36 | 0.51 | 2.21 | 1.71E−03 | 3.92E−02 |
| associated | | | | | | | |
| polypeptide 2, | | | | | | | |
| isoforms | | | | | | | |
| beta/gamma | | | | | | | |
| Aldose reductase | AKR1B1 | P15121 | 1.33 | 0.49 | 2.17 | 1.91E−03 | 3.92E−02 |
| Kallikrein-10 | KLK10 | O43240 | 1.31 | 0.5 | 2.12 | 1.56E−03 | 3.82E−02 |
| Programmed cell | PDCD5 | O14737 | 1.3 | 0.5 | 2.1 | 1.42E−03 | 3.66E−02 |
| death protein 5 | | | | | | | |
| Interleukin-12 | IL12B | P29460 | 1.3 | 0.58 | 2.01 | 3.85E−04 | 1.82E−02 |
| subunit beta | | | | | | | |
| 60S ribosomal | RPL12 | P30050 | | | | | |
| protein L12 | | | 1.27 | 0.5 | 2.03 | 1.15E−03 | 3.35E−02 |
| C-C motif | CCL14 | Q16627 | 1.26 | 0.67 | 1.85 | 2.78E−05 | 7.70E−03 |
| chemokine 14 | | | | | | | |
| CUB domain- | CDCP1 | Q9H5V8 | 1.24 | 0.59 | 1.88 | 1.74E−04 | 1.59E−02 |
| containing protein 1 | | | | | | | |
| Malignant T-cell- | MCTS1 | Q9ULC4 | 1.2 | 0.45 | 1.96 | 1.89E−03 | 3.92E−02 |
| amplified | | | | | | | |
| sequence 1 | | | | | | | |
| Troponin T, | TNNT2 | P45379 | 1.19 | 0.57 | 1.82 | 1.97E−04 | 1.59E−02 |
| cardiac muscle | | | | | | | |
| Tudor-interacting | NUDT16L1 | Q9BRJ7 | 1.19 | 0.47 | 1.91 | 1.24E−03 | 3.41E−02 |
| repair regulator | | | | | | | |
| protein | | | | | | | |
| C-X-C motif | CXCL10 | P02778 | 1.14 | 0.47 | 1.8 | 9.06E−04 | 3.07E−02 |
| chemokine 10 | | | | | | | |
| Thioredoxin | TXNRD1 | Q16881 | 1.09 | 0.42 | 1.77 | 1.39E−03 | 3.66E−02 |
| reductase 1, | | | | | | | |
| cytoplasmic | | | | | | | |
| E-selectin | SELE | P16581 | 1.03 | 0.37 | 1.69 | 2.35E−03 | 4.33E−02 |
| Tapasin | TAPBP | O15533 | 0.88 | 0.45 | 1.32 | 7.77E−05 | 1.10E−02 |
| Troponin I, fast | TNNI2 | P48788 | 0.77 | 0.35 | 1.2 | 3.38E−04 | 1.80E−02 |
| skeletal muscle | | | | | | | |
| Cadherin- | CDH11 | P55287 | 0.66 | 0.3 | 1.02 | 3.34E−04 | 1.80E−02 |
| 11: Extracellular | | | | | | | |
| domain | | | | | | | |
| Zymogen granule | ZG16 | O60844 | 0.18 | 0.08 | 0.28 | 2.43E−04 | 1.59E−02 |
| membrane | | | | | | | |
| protein 16 | | | | | | | |
| CD209 antigen | CD209 | Q9NNX6 | −1.13 | −1.74 | −0.51 | 3.60E−04 | 1.81E−02 |
| Interleukin-6 | IL6R | P08887 | −1.41 | −2.32 | −0.5 | 2.39E−03 | 4.33E−02 |
| receptor subunit | | | | | | | |
| alpha | | | | | | | |
| Platelet-activating | PLA2G7 | Q13093 | −1.52 | −2.32 | −0.72 | 2.09E−04 | 1.59E−02 |
| factor | | | | | | | |
| acetylhydrolase | | | | | | | |
| Matrix- | MXRA8 | Q9BRK3 | −1.92 | −3.12 | −0.71 | 1.93E−03 | 3.92E−02 |
| remodeling- | | | | | | | |
| associated protein | | | | | | | |
| 8: Extracellular | | | | | | | |
| domain | | | | | | | |
| Sodium-coupled | SLC5A8 | Q8N695 | −1.96 | −2.98 | −0.94 | 1.73E−04 | 1.59E−02 |
| monocarboxylate | | | | | | | |
| transporter 1 | | | | | | | |
| tRNA (guanine- | METTL1 | Q9UBP6 | −1.96 | −3.23 | −0.69 | 2.55E−03 | 4.52E−02 |
| N(7)-)- | | | | | | | |
| methyltransferase | | | | | | | |
| Insulin-like | IGFBP3 | P17936 | −2.07 | −3.39 | −0.74 | 2.25E−03 | 4.27E−02 |
| growth factor- | | | | | | | |
| binding protein 3 | | | | | | | |
| Insulin-like | IGFALS | P35858 | −2.25 | −3.64 | −0.86 | 1.57E−03 | 3.82E−02 |
| growth factor- | | | | | | | |
| binding protein | | | | | | | |
| complex acid | | | | | | | |
| labile subunit | | | | | | | |
| Histone | HAT1 | O14929 | −2.26 | −3.67 | 0.85 | 1.67E−03 | 3.92E−02 |
| acetyltransferase | | | | | | | |
| type B catalytic | | | | | | | |
| subunit | | | | | | | |
| Vitamin K- | PROC | P04070 | −2.72 | −4.36 | −1.08 | 1.17E−03 | 3.35E−02 |
| dependent | | | | | | | |
| protein C | | | | | | | |
| Cytoskeleton- | CKAP2 | Q8WWK9 | −2.75 | −4.48 | −1.02 | 1.82E−03 | 3.92E−02 |
| associated protein 2 | | | | | | | |
| Afamin | AFM | P43652 | −2.79 | −4.29 | −1.29 | 2.71E−04 | 1.65E−02 |

Of these, 19 proteins (19 GeneIDs) were shared between the older ever-smoker subset and the never-smoker subset; the magnitudes of effects were greater in the older ever-smokers, demonstrating that smoking status and advanced age are important effect modifiers in the associations of each of the 19 proteins with QIA (Table 12).

TABLE 12
Proteins associated with QIA shared amongst COPDGene,
CARDIA older smokers, and CARDIA nonsmokers

| | | | CARDIA older smokers | | | CARDIA never-smokers | | |
|---|---|---|---|---|---|---|---|---|
| | | | Percent | | | Percent | | |
| | Entrez | | QIA per | Upper | Lower | QIA per | Upper | Lower |
| Protein name | gene name | UniProt ID | ln(RFU) | 95% CI | 95% CI | ln(RFU) | 95% CI | 95% CI |
| Insulin-like growth factor- | IGFBP4 | P22692 | 3.8 | 2.12 | 5.48 | 1.96 | 0.73 | 3.19 |
| binding protein 4 | | | | | | | | |
| Tumor necrosis factor ligand | TNFSF15 | O95150 | 3.28 | 1.54 | 5.03 | 2.02 | 0.7 | 3.34 |
| superfamily member 15 | | | | | | | | |
| Heat shock 70 kDa protein | HSPA1A | P0DMV8 | 2.43 | 0.84 | 4.01 | 2.12 | 0.9 | 3.35 |
| 1A | | | | | | | | |
| Calsyntenin-3 | CLSTN3 | Q9BQT9 | 2.41 | 1.21 | 3.62 | 1.41 | 0.66 | 2.17 |
| Lamina-associated | TMPO | P42167 | 2.35 | 1.28 | 3.43 | 1.36 | 0.51 | 2.21 |
| polypeptide 2, isoforms | | | | | | | | |
| beta/gamma | | | | | | | | |
| C-X-C motif chemokine 13 | CXCL13 | O43927 | 2.25 | 1.03 | 3.46 | 1.69 | 0.89 | 2.5 |
| Troponin T, cardiac muscle | TNNT2 | P45379 | 2.18 | 1.14 | 3.21 | 1.19 | 0.57 | 1.82 |
| Macrophage-capping protein | CAPG | P40121 | 2.15 | 1.13 | 3.17 | 1.55 | 0.81 | 2.3 |
| CUB domain-containing | CDCP1 | Q9H5V8 | 2.11 | 1.3 | 2.92 | 1.24 | 0.59 | 1.88 |
| protein 1 | | | | | | | | |
| Kallikrein-10 | KLK10 | O43240 | 1.93 | 0.74 | 3.12 | 1.31 | 0.5 | 2.12 |
| Programmed cell death | PDCD5 | O14737 | 1.67 | 0.44 | 2.9 | 1.3 | 0.5 | 2.1 |
| protein 5 | | | | | | | | |
| Thioredoxin reductase 1, | TXNRD1 | Q16881 | 1.51 | 0.46 | 2.56 | 1.09 | 0.42 | 1.77 |
| cytoplasmic | | | | | | | | |
| E-selectin | SELE | P16581 | 1.44 | 0.51 | 2.38 | 1.03 | 0.37 | 1.69 |
| C-X-C motif chemokine 10 | CXCL10 | P02778 | 1.27 | 0.45 | 2.1 | 1.14 | 0.47 | 1.8 |
| Troponin I, fast skeletal | TNNI2 | P48788 | 1.26 | 0.56 | 1.96 | 0.77 | 0.35 | 1.2 |
| muscle | | | | | | | | |
| Cadherin-11: Extracellular | CDH11 | P55287 | 1.2 | 0.67 | 1.72 | 0.66 | 0.3 | 1.02 |
| domain | | | | | | | | |
| Zymogen granule | ZG16 | O60844 | 0.28 | 0.14 | 0.42 | 0.18 | 0.08 | 0.28 |
| membrane protein 16 | | | | | | | | |
| Interleukin-6 receptor | IL6R | P08887 | −2.14 | −3.53 | −0.76 | −1.41 | −2.32 | −0.5 |
| subunit alpha | | | | | | | | |
| Matrix-remodeling- | MXRA8 | Q9BRK3 | −2.78 | −4.42 | −1.13 | −1.92 | −3.12 | −0.71 |
| associated protein | | | | | | | | |
| 8: Extracellular domain | | | | | | | | |

Lastly, analysis of the full CARDIA 7K assay, which contains 2,356 additional unique aptamers not available in the COPDGene 5K assay, yielded 68 additional unique proteins (67 GeneIDs) that were significantly associated with QIA after adjustment for covariates (Table 13). These associations were not entered into the pathway enrichment analysis, as they need to be validated in other cohorts

TABLE 13
Multivariable regression analysis of QIA in CARDIA, unique to 7k panel

| | | | Percent | Upper | Lower | | |
|---|---|---|---|---|---|---|---|
| | Entrez | | QLA per | 95% | 95% | p- | FDR p- |
| Protein name | gene name | UniProt ID | ln(RFU) | CI | CI | value | value |
| Ubiquitin-like protein ISG15 | ISG15 | P05161 | 1.95 | 1.27 | 2.62 | 1.66E−08 | 2.10E−05 |
| Metalloproteinase inhibitor 1 | TIMP1 | P01033 | 1.9 | 0.95 | 2.85 | 9.18E−05 | 6.80E−03 |
| Apoptosis-associated speck- | PYCARD | Q9ULZ3 | 1.78 | 0.79 | 2.77 | 4.37E−04 | 2.02E−02 |
| like protein containing a | | | | | | | |
| CARD | | | | | | | |
| Vascular endothelial growth | FLT4 | P35916 | 1.7 | 0.99 | 2.42 | 3.27E−06 | 8.57E−04 |
| factor receptor 3 | | | | | | | |
| Thy-1 membrane | THY1 | P04216 | 1.65 | 0.95 | 2.35 | 4.10E−06 | 9.69E−04 |
| glycoprotein | | | | | | | |
| Serine/threonine-protein | PPP1R10 | Q96QC0 | 1.5 | 0.76 | 2.23 | 6.39E−05 | 5.58E−03 |
| phosphatase 1 regulatory | | | | | | | |
| subunit 10 | | | | | | | |
| Synembryn-A | RIC8A | Q9NPQ8 | 1.49 | 0.71 | 2.27 | 1.80E−04 | 1.08E−02 |
| Tumor necrosis factor | RELT | Q969ZA | 1.49 | 0.62 | 2.35 | 7.47E−04 | 2.99E−02 |
| receptor superfamily member | | | | | | | |
| 19L | | | | | | | |
| Macrophage receptor | MARCO | Q9UEW3 | 1.4 | 0.59 | 2.21 | 6.73E−04 | 2.76E−02 |
| MARCO | | | | | | | |
| Z-DNA-binding protein 1 | ZBP1 | Q9H171 | 1.4 | 0.76 | 2.04 | 1.89E−05 | 2.63E−03 |
| Complement component C7 | C7 | P10643 | 1.39 | 0.57 | 2.2 | 8.58E−04 | 3.26E−02 |
| No protein | NA | NA | 1.34 | 0.63 | 2.05 | 2.18E−04 | 1.25E−02 |
| C-C motif chemokine 22 | CCL22 | O00626 | 1.34 | 0.72 | 1.96 | 2.00E−05 | 2.67E−03 |
| C-C motif chemokine 7 | CCL7 | P80098 | 1.33 | 0.75 | 1.91 | 7.01E−06 | 1.35E−03 |
| Marginal zone B- and B1- | MZB1 | Q8WU39 | 1.31 | 0.6 | 2.02 | 3.11E−04 | 1.57E−02 |
| cell-specific protein | | | | | | | |
| Complement component C9 | C9 | P02748 | 1.22 | 0.5 | 1.93 | 8.62E−04 | 3.26E−02 |
| Endothelial cell-derived | LIPG | Q9Y5X9 | 1.21 | 0.5 | 1.91 | 8.06E−04 | 3.13E−02 |
| lipase | | | | | | | |
| Scavenger receptor class B | SCARB1 | Q8WTV0 | 1.2 | 0.68 | 1.72 | 6.46E−06 | 1.33E−03 |
| member 1 | | | | | | | |
| cAMP-specific 3′,5′-cyclic | PDE4C | Q08493 | 1.19 | 0.62 | 1.76 | 4.67E−05 | 4.51E−03 |
| phosphodiesterase 4C | | | | | | | |
| C-X-C motif chemokine 11 | CXCL11 | O14625 | 1.18 | 0.46 | 1.91 | 1.33E−03 | 4.27E−02 |
| Butyrophilin subfamily 3 | BTN3A2 | P78410 | 1.17 | 0.59 | 1.75 | 7.01E−05 | 5.91E−03 |
| member A2 | | | | | | | |
| Inositol-tetrakisphosphate 1- | ITPK1 | Q13572 | 1.16 | 0.65 | 1.67 | 8.24E−06 | 1.55E−03 |
| kinase | | | | | | | |
| Succinate dehydrogenase | SDHB | P21912 | 1.15 | 0.5 | 1.8 | 5.25E−04 | 2.35E−02 |
| [ubiquinone] iron-sulfur | | | | | | | |
| subunit, mitochondrial | | | | | | | |
| Interleukin-22 | IL22 | Q9GZX6 | 1.11 | 0.45 | 1.77 | 1.05E−03 | 3.63E−02 |
| V-set and immunoglobulin | VSIG4 | Q9Y279 | 1.1 | 0.44 | 1.75 | 1.06E−03 | 3.63E−02 |
| domain-containing protein 4 | | | | | | | |
| Protein CutA | CUTA | O60888 | 1.1 | 0.43 | 1.77 | 1.27E−03 | 4.12E−02 |
| Cystatin B | CSTB | P04080 | 1.09 | 0.41 | 1.77 | 1.67E−03 | 4.96E−02 |
| C-C motif chemokine 15 | CCL15 | Q16663 | 1.01 | 0.41 | 1.6 | 8.86E−04 | 3.31E−02 |
| Small RNA 2′-O- | HENMT1 | Q5T819 | 0.96 | 0.4 | 1.52 | 7.86E−04 | 3.10E−02 |
| methyltransferase | | | | | | | |
| Transcription initiation factor | TAF12 | Q16514 | 0.94 | 0.56 | 1.32 | 1.33E−06 | 4.89E−04 |
| TFIID subunit 12 | | | | | | | |
| Transcription elongation | TCEAL5 | Q5H9L2 | 0.92 | 0.37 | 1.48 | 1.06E−03 | 3.63E−02 |
| factor A protein-like 5 | | | | | | | |
| Centrin-1 | CETN1 | Q12798 | 0.91 | 0.47 | 1.36 | 5.66E−05 | 5.13E−03 |
| Myosin-binding protein C, | MYBPC2 | Q14324 | 0.82 | 0.32 | 1.33 | 1.48E−03 | 4.60E−02 |
| fast-type | | | | | | | |
| Eukaryotic translation | EIF4EBP2 | Q13542 | 0.79 | 0.3 | 1.27 | 1.43E−03 | 4.47E−02 |
| initiation factor 4E-binding | | | | | | | |
| protein 2 | | | | | | | |
| Cathepsin H | CTSH | P09668 | 0.79 | 0.41 | 1.18 | 6.12E−05 | 5.48E−03 |
| GTP cyclohydrolase I | GCHFR | P30047 | 0.75 | 0.29 | 1.22 | 1.52E−03 | 4.68E−02 |
| feedback regulatory protein | | | | | | | |
| PDZ and LIM domain protein | PDLIM3 | Q53GG5 | 0.75 | 0.35 | 1.14 | 2.14E−04 | 1.23E−02 |
| 3 | | | | | | | |
| C−>U-editing enzyme | APOBEC2 | Q9Y235 | 0.71 | 0.33 | 1.09 | 2.64E−04 | 1.43E−02 |
| APOBEC-2 | | | | | | | |
| Desmoglein-4 | DSG4 | Q86SJ6 | 0.7 | 0.43 | 0.97 | 6.16E−07 | 3.23E−04 |
| Myosin regulatory light chain | MYLPF | Q96A32 | 0.68 | 0.28 | 1.08 | 8.54E−04 | 3.26E−02 |
| 2, skeletal muscle isoform | | | | | | | |
| Homeobox protein TGIF2LX | TGIF2LX | Q8IUE1 | 0.66 | 0.26 | 1.06 | 1.32E−03 | 4.25E−02 |
| Alpha-L-fucoside | FUCA1 | P04066 | 0.61 | 0.27 | 0.94 | 3.74E−04 | 1.82E−02 |
| fucohydrolase | | | | | | | |
| Filamin-A: Calponin | FLNA | P21333 | 0.54 | 0.22 | 0.86 | 1.05E−03 | 3.63E−02 |
| Homology 1 | | | | | | | |
| Serum amyloid A-1 protein | SAA1 | PODJI8 | 0.35 | 0.21 | 0.5 | 2.45E−06 | 6.92E−04 |
| Beta-1,4-mannosyl- | MGAT3 | Q09327 | −0.82 | −1.29 | −0.34 | 7.41E−04 | 2.99E−02 |
| glycoprotein 4-beta-N- | | | | | | | |
| acetylglucosaminyltransferase | | | | | | | |
| Protein FAM189A2 | FAM189A2 | Q15884 | −0.85 | −1.32 | −0.37 | 4.92E−04 | 2.23E−02 |
| Protein Wnt-5b | WNT5B | Q9H1J7 | −1.08 | −1.73 | −0.43 | 1.09E−03 | 3.69E−02 |
| FRAS1-related extracellular | FREM1 | Q5H8C1 | −1.11 | −1.79 | −0.44 | 1.20E−03 | 3.94E−02 |
| matrix protein 1 | | | | | | | |
| Tubulin polymerization- | TPPP3 | Q9BW30 | −1.32 | −2.1 | −0.53 | 1.06E−03 | 3.63E−02 |
| promoting protein family | | | | | | | |
| member 3 | | | | | | | |
| Integrin a11b1 | ITGA11\|ITGB1 | Q9UKX5\|P05556 | −1.35 | −1.91 | −0.8 | 1.76E−06 | 6.16E−04 |
| Ephrin type-A receptor 6 | EPHA6 | Q9UF33 | −1.39 | −2.08 | −0.7 | 8.57E−05 | 6.55E−03 |
| Integrin alpha V beta 3 | ITGAV\|ITGB3 | P06756\|P05106 | −1.4 | −2.08 | −0.71 | 7.40E−05 | 5.98E−03 |
| Interleukin-6 receptor subunit | IL6R | P08887 | −1.42 | −2.19 | −0.66 | 2.67E−04 | 1.44E−02 |
| alpha | | | | | | | |
| Mitogen-activated protein | MAP3K11 | Q16584 | −1.48 | −2.28 | −0.68 | 2.88E−04 | 1.51E−02 |
| kinase kinase kinase 11 | | | | | | | |
| Contactin-3 | CNTN3 | Q9P232 | −1.54 | −2.31 | −0.77 | 9.42E−05 | 6.91E−03 |
| Ciliary neurotrophic factor | CNTFR | P26992 | −1.61 | −2.37 | −0.86 | 2.87E−05 | 3.36E−03 |
| receptor subunit alpha | | | | | | | |
| Probable phosphoglycerate | PGAM4 | Q8N0Y7 | −1.62 | −2.63 | −0.61 | 1.66E−03 | 4.96E−02 |
| mutase 4 | | | | | | | |
| Interleukin-6 receptor subunit | IL6R | P08887 | −1.66 | −2.44 | −0.89 | 2.73E−05 | 3.34E−03 |
| alpha | | | | | | | |
| Activated Protein C | PROC | P04070 | −1.78 | −2.86 | −0.7 | 1.27E−03 | 4.12E−02 |
| Complement C1q-like protein | C1QL3 | Q5VWW1 | −1.81 | −2.58 | −1.05 | 3.65E−06 | 8.91E−04 |
| 3 | | | | | | | |
| G1/S-specific cyclin-E1 | CCNE1 | P24864 | −1.86 | −2.95 | −0.76 | 9.10E−04 | 3.38E−02 |
| Ubiquitin-conjugating | UBE2R2 | Q712K3 | −1.9 | −3.04 | −0.76 | 1.07E−03 | 3.63E−02 |
| enzyme E2 R2 | | | | | | | |
| Speriolin-like protein | SPATC1L | Q9H0A9 | −1.97 | −2.97 | 0.97 | 1.19E−04 | 8.27E−03 |
| Gamma-glutamyltransferase | GGT5 | P36269 | −2.16 | −3.15 | −1.18 | 1.67E−05 | 2.40E−03 |
| 5 | | | | | | | |
| Afamin | AFM | P43652 | −2.31 | −3.41 | −1.21 | 4.06E−05 | 4.04E−03 |
| Cholinesterase | BCHE | P06276 | −2.51 | −3.47 | −1.56 | 2.57E−07 | 1.57E−04 |
| Prostasin | PRSS8 | Q16651 | −2.58 | −3.86 | −1.31 | 7.35E−05 | 5.98E−03 |
| Beta-hexosaminidase subunit | HEXB | P07686 | −3.04 | −4.4 | −1.68 | 1.26E−05 | 1.93E−03 |
| beta | | | | | | | |

1. A method comprising quantifying, within a biological sample from a subject that is a current or former tobacco smoker, the levels of two or more biomarkers selected from: Complement factor H; Fibrinogen; Complement factor B; Antileukoproteinase; Neurofascin; Neuropilin-2; Fibrinogen gamma chain; Insulin-like growth factor-binding protein 4; C—X—C motif chemokine 16; Atrial natriuretic factor; Serine/arginine-rich splicing factor 7; Tumor necrosis factor ligand superfamily member 15; Serine/arginine-rich splicing factor 6; RNA-binding protein EWS; Immunoglobulin G; Serpin B8; Leukocyte immunoglobulin-like receptor subfamily A member 5; Cystatin-C; Nidogen-1; Gamma-glutamyl hydrolase; Complement C1q tumor necrosis factor-related protein 1; Insulin-like growth factor-binding protein 7; Plastin-2; Desmocollin-2; Ganglioside GM2 activator; Fibulin-5; Ephrin-A4; Heat shock 70 kDa protein 1B; Spondin-1; Acidic leucine-rich nuclear phosphoprotein 32 family member B; Peptidyl-prolyl cis-trans isomerase C; B melanoma antigen 2; Collagen alpha-1(XXVIII) chain; Oncoprotein-induced transcript 3 protein; Collagen alpha-3(VI) chain:Bovine pancreatic trypsin inhibitor/Kunitz inhibitor domain, isoform 1; Transmembrane emp24 domain-containing protein 10; Delta-like protein 1; Endoplasmic reticulum lectin 1; C—C motif chemokine 21; Tumor necrosis factor receptor superfamily member 17; Thioredoxin domain-containing protein 5; Beta-2-microglobulin; Heat shock cognate 71 kDa protein; WAP four-disulfide core domain protein 2; Heat shock 70 kDa protein 1A; Tolloid-like protein 1; Triggering receptor expressed on myeloid cells 1; Calsyntenin-3; Lipopolysaccharide-binding protein; Lamina-associated polypeptide 2, isoforms beta/gamma; Erythropoietin; Ephrin type-B receptor 2; C—X—C motif chemokine 13; Tryptophan—tRNA ligase, cytoplasmic; Folate receptor beta; Holo-Transcobalamin-2; NTF2-related export protein 1; Protein ABHD14A; C—C motif chemokine 18; Semaphorin-6B; Retinoic acid receptor responder protein 2; Plexin-D1; Troponin T, cardiac muscle; Macrophage-capping protein; Syntaxin-12; Interleukin-9; CUB domain-containing protein 1; Vesicular integral-membrane protein VIP36; Fibroblast growth factor 23; BMP and activin membrane-bound inhibitor homolog:Extracellular domain; Marginal zone B- and B1-cell-specific protein; Leukocyte immunoglobulin-like receptor subfamily A member 5; Interferon-induced GTP-binding protein Mx1; Sushi, von Willebrand factor type A, EGF and pentraxin domain-containing protein 1:EGF-like domains 4-6; Intercellular adhesion molecule 1; Ephrin type-A receptor 2; Transferrin receptor protein 1:Extracellular domain; Trefoil factor 3; Kallikrein-10; Alpha-1-antichymotrypsin complex; Neuroblastoma suppressor of tumorigenicity 1; Vascular endothelial growth factor D; Small ubiquitin-related modifier 2; T-cell immunoglobulin and mucin domain-containing protein 4; Ribonuclease K6; IGF-like family receptor 1; Sushi, von Willebrand factor type A, EGF and pentraxin domain-containing protein 1:Sushi 15-18; Elafin; ADAMTS-like protein 2; Transformer-2 protein homolog beta; Protein CYR61; Lysozyme C; Guanylate-binding protein 1; Macrophage metalloelastase; Angiopoietin-2; Axin-2; WAP four-disulfide core domain protein 1; Complement C3d fragment; DnaJ homolog subfamily B member 12; Prostaglandin-H2 D-isomerase; Interleukin-1 receptor antagonist protein; Programmed cell death protein 5; Regenerating islet-derived protein 3-alpha; Lactoperoxidase; SCP2 sterol-binding domain-containing protein 1; Trefoil factor 3; C—C motif chemokine 19; Granulocyte colony-stimulating factor; Phospholipase A2, membrane associated; Trefoil factor 2; Gamma-interferon-inducible protein 16:Isoform 2, Hematopoietic expression, interferon-inducible nature, and nuclear localization 1; Acyl-CoA-binding protein; Thioredoxin reductase 1, cytoplasmic; Interleukin-3 receptor subunit alpha; Thrombospondin-2; Tumor necrosis factor receptor superfamily member 1B; Golgi membrane protein 1; Growth/differentiation factor 15; Kallikrein-13; Induced myeloid leukemia cell differentiation protein Mcl-1; E-selectin; Macrophage scavenger receptor types I and II:Extracellular domain; Ribonuclease pancreatic; Gastrokine-2; Receptor-transporting protein 4; Ubiquitin-like protein ISG15; C—C motif chemokine 15; Copper transport protein ATOX1; C—X—C motif chemokine 10; 60S ribosomal protein L30; Troponin I, fast skeletal muscle; Cadherin-11:Extracellular domain; Syntaxin-8; Fibrinogen-like protein 1; Serum amyloid A-2 protein; Chitotriosidase-1; Platelet-derived growth factor receptor-like protein; BPI fold-containing family B member 1; N-terminal pro-BNP; Beta-defensin 4A; C-reactive protein; Serum amyloid A-1 protein; Zymogen granule membrane protein 16; Signal peptide, CUB and EGF-like domain-containing protein 1; Glypican-3; Chondrocalcin; Protein MENT; Histone-lysine N-methyltransferase EHMT2; Sodium/potassium-transporting ATPase subunit beta-1; Neurocan core protein; Cartilage intermediate layer protein 2; Group XIIB secretory phospholipase A2-like protein; Ficolin-3; Fibroblast growth factor receptor 3:Extracellular domain; Muellerian-inhibiting factor; Solute carrier family 22 member 16; Interleukin-6 receptor subunit alpha; Proprotein convertase subtilisin/kexin type 7; Anthrax toxin receptor 1; Inositol monophosphatase 3; Thrombin; Neural cell adhesion molecule 1, 120 kDa isoform; Apolipoprotein M; Receptor-type tyrosine-protein phosphatase delta; Cell adhesion molecule-related/down-regulated by oncogenes; Ciliary neurotrophic factor receptor subunit alpha; E3 ubiquitin-protein ligase RNF146; N-acetylmuramoyl-L-alanine amidase; Matrix-remodeling-associated protein 8:Extracellular domain; Growth/differentiation factor 2; Platelet endothelial aggregation receptor 1:Extracellular domain; Myosin regulatory light chain 2, atrial isoform; Hedgehog-interacting protein; Carbonic anhydrase 4; Prostaglandin F2 receptor negative regulator; E3 ubiquitin-protein ligase DTX1; Interleukin-1 receptor type 2; Receptor-type tyrosine-protein phosphatase eta; NT-3 growth factor receptor; Leucine-rich repeats and immunoglobulin-like domains protein 3; Beta-1,4-galactosyltransferase 6; Semaphorin-3G; Plasma kallikrein; Collagen alpha-1(XIII) chain; Alpha-2-HS-glycoprotein; Bifunctional heparan sulfate N-deacetylase/N-sulfotransferase 1; Contactin-1; Cholinesterase; Heparan-sulfate 6-O-sulfotransferase 3; Coxsackievirus and adenovirus receptor; B melanoma antigen 3; BDNF/NT-3 growth factors receptor; Contactin-4; N-acetylglucosamine-1-phosphodiester alpha-N-acetylglucosaminidase; Neuropeptide S; Alpha-2-antiplasmin; Mannosyl-oligosaccharide 1,2-alpha-mannosidase IB; and Epidermal growth factor receptor.

2. The method of claim 1, wherein the two or more biomarkers are selected from: Insulin-like growth factor-binding protein 4; Tumor necrosis factor ligand superfamily member 15; Heat shock 70 kDa protein 1A; Calsyntenin-3; Lamina-associated polypeptide 2, isoforms beta/gamma; C—X—C motif chemokine 13; Troponin T, cardiac muscle; Macrophage-capping protein; CUB domain-containing protein 1; Kallikrein-10; Programmed cell death protein 5; Thioredoxin reductase 1, cytoplasmic; E-selectin; C—X—C motif chemokine 10; Troponin I, fast skeletal muscle; Cadherin-11:Extracellular domain; Zymogen granule membrane protein 16; Interleukin-6 receptor subunit alpha; and Matrix-remodeling-associated protein 8:Extracellular domain.

3. The method claim 1, wherein the two or more biomarkers are 200 or fewer biomarkers.

4. The method of claim 1, wherein the two or more biomarkers are a protein or a nucleic acid.

5-9. (canceled)

10. The method of claim 1, wherein the biological sample is plasma or blood.

11. The method of claim 1, wherein the biological sample is obtained from a subject that suffers from impaired respiratory health.

12. The method of claim 1, wherein the biological sample is obtained from a subject that is at risk of impaired respiratory health.

13-17. (canceled)

18. A method comprising quantifying, within a biological sample from a subject that is not a current or former tobacco smoker, the levels of two or more biomarkers selected from: HLA class I histocompatibility antigen, alpha chain G; Receptor-interacting serine/threonine-protein kinase 2; Lumican; Matrix Gla protein; Heat shock 70 kDa protein 1A; Switch-associated protein 70; Tumor necrosis factor ligand superfamily member 15; Insulin-like growth factor-binding protein 4; Ran-binding protein 3; Laminin subunit gamma-2; C—X—C motif chemokine 13; Thrombospondin-4; Tripeptidyl-peptidase 1; Macrophage-capping protein; Thrombospondin-3; Myosin light chain 6B; Torsin-1A-interacting protein 1:Perinuclear domain; Calsyntenin-3; Lamina-associated polypeptide 2, isoforms beta/gamma; Aldose reductase; Kallikrein-10; Programmed cell death protein 5; Interleukin-12 subunit beta; 60S ribosomal protein L12; C—C motif chemokine 14; CUB domain-containing protein 1; Malignant T-cell-amplified sequence 1; Troponin T, cardiac muscle; Tudor-interacting repair regulator protein; C—X—C motif chemokine 10; Thioredoxin reductase 1, cytoplasmic; E-selectin; Tapasin; Troponin I, fast skeletal muscle; Cadherin-11:Extracellular domain; Zymogen granule membrane protein 16; CD209 antigen; Interleukin-6 receptor subunit alpha; Platelet-activating factor acetylhydrolase; Matrix-remodeling-associated protein 8:Extracellular domain; Sodium-coupled monocarboxylate transporter 1; tRNA (guanine-N(7)-)-methyltransferase; Insulin-like growth factor-binding protein 3; Insulin-like growth factor-binding protein complex acid labile subunit; Histone acetyltransferase type B catalytic subunit; Vitamin K-dependent protein C; Cytoskeleton-associated protein 2; and Afamin.

19. The method of claim 18, wherein the two or more biomarkers are selected from: Insulin-like growth factor-binding protein 4; Tumor necrosis factor ligand superfamily member 15; Calsyntenin-3; Lamina-associated polypeptide 2, isoforms beta/gamma; C—X—C motif chemokine 13; Troponin T, cardiac muscle; Macrophage-capping protein; CUB domain-containing protein 1; Kallikrein-10; Programmed cell death protein 5; Thioredoxin reductase 1, cytoplasmic; E-selectin; C—X—C motif chemokine 10; Troponin I, fast skeletal muscle; Cadherin-11:Extracellular domain; Zymogen granule membrane protein 16; Interleukin-6 receptor subunit alpha; and Matrix-remodeling-associated protein 8:Extracellular domain.

20. The method of claim 18, wherein the two or more biomarkers are a protein or a nucleic acid.

21-25. (canceled)

26. The method of claim 18, wherein the biological sample is plasma or blood.

27. The method of claim 18, wherein the biological sample is obtained from a subject that suffers from impaired respiratory health.

28. The method of one of claim 18, wherein the biological sample is obtained from a subject that is at risk of impaired respiratory health.

29-33. (canceled)

34. A method of assessing the lung health of a subject comprising:
(a) quantifying, within a biological sample from a subject, the levels of one or more biomarkers selected from: Complement factor H; Fibrinogen; Complement factor B; Antileukoproteinase; Neurofascin; Neuropilin-2; Fibrinogen gamma chain; Insulin-like growth factor-binding protein 4; C—X—C motif chemokine 16; Atrial natriuretic factor; Serine/arginine-rich splicing factor 7; Tumor necrosis factor ligand superfamily member 15; Serine/arginine-rich splicing factor 6; RNA-binding protein EWS; Immunoglobulin G; Serpin B8; Leukocyte immunoglobulin-like receptor subfamily A member 5; Cystatin-C; Nidogen-1; Gamma-glutamyl hydrolase; Complement C1q tumor necrosis factor-related protein 1; Insulin-like growth factor-binding protein 7; Plastin-2; Desmocollin-2; Ganglioside GM2 activator; Fibulin-5; Ephrin-A4; Heat shock 70 kDa protein 1B; Spondin-1; Acidic leucine-rich nuclear phosphoprotein 32 family member B; Peptidyl-prolyl cis-trans isomerase C; B melanoma antigen 2; Collagen alpha-1(XXVIII) chain; Oncoprotein-induced transcript 3 protein; Collagen alpha-3(VI) chain:Bovine pancreatic trypsin inhibitor/Kunitz inhibitor domain, isoform 1; Transmembrane emp24 domain-containing protein 10; Delta-like protein 1; Endoplasmic reticulum lectin 1; C—C motif chemokine 21; Tumor necrosis factor receptor superfamily member 17; Thioredoxin domain-containing protein 5; Beta-2-microglobulin; Heat shock cognate 71 kDa protein; WAP four-disulfide core domain protein 2; Heat shock 70 kDa protein 1A; Tolloid-like protein 1; Triggering receptor expressed on myeloid cells 1; Calsyntenin-3; Lipopolysaccharide-binding protein; Lamina-associated polypeptide 2, isoforms beta/gamma; Erythropoietin; Ephrin type-B receptor 2; C—X—C motif chemokine 13; Tryptophan—tRNA ligase, cytoplasmic; Folate receptor beta; Holo-Transcobalamin-2; NTF2-related export protein 1; Protein ABHD14A; C—C motif chemokine 18; Semaphorin-6B; Retinoic acid receptor responder protein 2; Plexin-D1; Troponin T, cardiac muscle; Macrophage-capping protein; Syntaxin-12; Interleukin-9; CUB domain-containing protein 1; Vesicular integral-membrane protein VIP36; Fibroblast growth factor 23; BMP and activin membrane-bound inhibitor homolog:Extracellular domain; Marginal zone B- and B1-cell-specific protein; Leukocyte immunoglobulin-like receptor subfamily A member 5; Interferon-induced GTP-binding protein Mx1; Sushi, von Willebrand factor type A, EGF and pentraxin domain-containing protein 1:EGF-like domains 4-6; Intercellular adhesion molecule 1; Ephrin type-A receptor 2; Transferrin receptor protein 1:Extracellular domain; Trefoil factor 3; Kallikrein-10; Alpha-1-antichymotrypsin complex; Neuroblastoma suppressor of tumorigenicity 1; Vascular endothelial growth factor D; Small ubiquitin-related modifier 2; T-cell immunoglobulin and mucin domain-containing protein 4; Ribonuclease K6; IGF-like family receptor 1; Sushi, von Willebrand factor type A, EGF and pentraxin domain-containing protein 1:Sushi 15-18; Elafin; ADAMTS-like protein 2; Transformer-2 protein homolog beta; Protein CYR61; Lysozyme C; Guanylate-binding protein 1; Macrophage metalloelastase; Angiopoietin-2; Axin-2; WAP four-disulfide core domain protein 1; Complement C3d fragment; DnaJ homolog subfamily B member 12; Prostaglandin-H2 D-isomerase; Interleukin-1 receptor antagonist protein; Programmed cell death protein 5; Regenerating islet-derived protein 3-alpha; Lactoperoxidase; SCP2 sterol-binding domain-containing protein 1; Trefoil factor 3; C—C motif chemokine 19; Granulocyte colony-stimulating factor; Phospholipase A2, membrane associated; Trefoil factor 2; Gamma-interferon-inducible protein 16:Isoform 2, Hematopoietic expression, interferon-inducible nature, and nuclear localization 1; Acyl-CoA-binding protein; Thioredoxin reductase 1, cytoplasmic; Interleukin-3 receptor subunit alpha; Thrombospondin-2; Tumor necrosis factor receptor superfamily member 1B; Golgi membrane protein 1; Growth/differentiation factor 15; Kallikrein-13; Induced myeloid leukemia cell differentiation protein Mcl-1; Macrophage scavenger receptor types I and II:Extracellular domain; Ribonuclease pancreatic; Gastrokine-2; Receptor-transporting protein 4; Ubiquitin-like protein ISG15; C—C motif chemokine 15; Copper transport protein ATOX1; C—X—C motif chemokine 10; 60S ribosomal protein L30; Troponin I, fast skeletal muscle; Cadherin-11:Extracellular domain; Syntaxin-8; Fibrinogen-like protein 1; Serum amyloid A-2 protein; Chitotriosidase-1; Platelet-derived growth factor receptor-like protein; BPI fold-containing family B member 1; N-terminal pro-BNP; Beta-defensin 4A; C-reactive protein; Serum amyloid A-1 protein; Zymogen granule membrane protein 16; Signal peptide, CUB and EGF-like domain-containing protein 1; Glypican-3; Chondrocalcin; Protein MENT; Histone-lysine N-methyltransferase EHMT2; Sodium/potassium-transporting ATPase subunit beta-1; Neurocan core protein; Cartilage intermediate layer protein 2; Group XIIB secretory phospholipase A2-like protein; Ficolin-3; Fibroblast growth factor receptor 3:Extracellular domain; Muellerian-inhibiting factor; Solute carrier family 22 member 16; Interleukin-6 receptor subunit alpha; Proprotein convertase subtilisin/kexin type 7; Anthrax toxin receptor 1; Inositol monophosphatase 3; Thrombin; Neural cell adhesion molecule 1, 120 kDa isoform; Apolipoprotein M; Receptor-type tyrosine-protein phosphatase delta; Cell adhesion molecule-related/down-regulated by oncogenes; Ciliary neurotrophic factor receptor subunit alpha; E3 ubiquitin-protein ligase RNF146; N-acetylmuramoyl-L-alanine amidase; Matrix-remodeling-associated protein 8:Extracellular domain; Growth/differentiation factor 2; Platelet endothelial aggregation receptor 1:Extracellular domain; Myosin regulatory light chain 2, atrial isoform; Hedgehog-interacting protein; Carbonic anhydrase 4; Prostaglandin F2 receptor negative regulator; E3 ubiquitin-protein ligase DTX1; Interleukin-1 receptor type 2; Receptor-type tyrosine-protein phosphatase eta; NT-3 growth factor receptor; Leucine-rich repeats and immunoglobulin-like domains protein 3; Beta-1,4-galactosyltransferase 6; Semaphorin-3G; Plasma kallikrein; Collagen alpha-1(XIII) chain; Alpha-2-HS-glycoprotein; Bifunctional heparan sulfate N-deacetylase/N-sulfotransferase 1; Contactin-1; Cholinesterase; Heparan-sulfate 6-O-sulfotransferase 3; Coxsackievirus and adenovirus receptor; B melanoma antigen 3; BDNF/NT-3 growth factors receptor; Contactin-4; N-acetylglucosamine-1-phosphodiester alpha-N-acetylglucosaminidase; Neuropeptide S; Alpha-2-antiplasmin; Mannosyl-oligosaccharide 1,2-alpha-mannosidase IB; Epidermal growth factor receptor; HLA class I histocompatibility antigen, alpha chain G; Receptor-interacting serine/threonine-protein kinase 2; Lumican; Matrix Gla protein; Switch-associated protein 70; Ran-binding protein 3; Laminin subunit gamma-2; Thrombospondin-4; Tripeptidyl-peptidase 1; Thrombospondin-3; Myosin light chain 6B; Torsin-1A-interacting protein 1:Perinuclear domain; Calsyntenin-3; Aldose reductase; Interleukin-12 subunit beta; 60S ribosomal protein L12; C—C motif chemokine 14; Malignant T-cell-amplified sequence 1; Tudor-interacting repair regulator protein; Tapasin; CD209 antigen; Platelet-activating factor acetylhydrolase; Sodium-coupled monocarboxylate transporter 1; tRNA (guanine-N(7)-)-methyltransferase; Insulin-like growth factor-binding protein 3; Insulin-like growth factor-binding protein complex acid labile subunit; Histone acetyltransferase type B catalytic subunit; Vitamin K-dependent protein C; Cytoskeleton-associated protein 2; Afamin; E-selectin; and
(b) comparing the level of the one or more biomarkers in (a) to a control or threshold value for each, wherein a significant difference between the level of one or more of the biomarkers from the control or threshold value is indicative of a impaired respiratory health.

35. The method of claim 34, further comprising measuring quantitative interstitial abnormalities (QIA) with a CT scan of the subject.

36. The method of claim 34, wherein the panel comprises 50 or fewer biomarkers.

37-49. (canceled)
